SEQUENCE LISTING (10 sequences)

SEQ ID NO: 1 - DNA, 48 bp, Human
gatctcgacgggcgcggatccaccatggattacaaggatgacgacgat

SEQ ID NO: 2 - DNA, 49 bp, Human
catggattacaaggatgacgacgataagatggctagtggcagctgtcag

SEQ ID NO: 3 - DNA, 47 bp, Human
gttcacgagatccactattcagtaagagttccaccaatttgggactg

SEQ ID NO: 4 - DNA, 23 bp, Human
gaatagtggatctcgtgaacaag

SEQ ID NO: 5 - DNA, 25 bp, Human
gtcagacaaactgcttggaaccagc

SEQ ID NO: 6 - DNA, 40 bp, Human
ctggttccaagcagtttgtctgaccacaggcctgtgatag

SEQ ID NO: 7 - DNA, 46 bp, Human
gttctagccaagcttggtaccctattactcaacagatgttcgtctc

SEQ ID NO: 8 - DNA, 7611 bp, Human
atggattacaaggatgacgacgataagatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatactggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggaattatttcgcgtagagatgtggaaaaatttctttcaaagaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 9 - protein, 2536 aa, Artificial Sequence
  Translated protein sequence for human G2019 full length LRRK2 flag tagged protein
MDYKDDDDKMASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 10 - protein, 15 aa, Artificial Sequence
  ′LRRKtide′ peptide
RLGRDKYKTLRQIRQ

(19) United States
(12) Patent Application Publication
(10) Pub. No.: US 2020/0392158 A1
(43) Pub. Date: Dec. 17, 2020

(54) INHIBITORS OF LEUCINE RICH REPEAT KINASE 2

(71) Applicant: GLAXOSMITHKLINE INTELLECTUAL PROPERTY DEVELOPMENT LIMITED, Brentford, Middlesex (GB)

(72) Inventors: Xiao DING, Shanghai (CN); Ming-Hsun HO, Collegeville, PA (US); Feng REN, Shanghai (CN); Haihua YU, Shanghai (CN); Yang ZHAN, Shanghai (CN)

(21) Appl. No.: 16/621,032

(22) PCT Filed: Jul. 12, 2018
(86) PCT No.: PCT/EP2018/069051

§ 371 Date: Dec. 10, 2019

(30) Foreign Application Priority Data
Jul. 14, 2017 (CN) PCT/CN2017/093024

(51) Int. Cl. C07D 498/18; C07D 519/00

(52) U.S. Cl. CPC C07D 498/18; C07D 519/00

(57) ABSTRACT

The present invention relates to novel compounds that inhibit LRRK2 kinase activity, to processes for their preparation, to compositions containing them and to their use in the treatment of or prevention of diseases associated with or characterized by LRRK2 kinase activity, for example Parkinson's disease, Alzheimer's disease and amyotrophic lateral sclerosis (ALS).

### FIELD OF THE INVENTION

The present invention relates to novel compounds that inhibit LRRK2 kinase activity, processes for their preparation, compositions containing them and their use in the treatment of diseases associated with or characterized by LRRK2 kinase activity, for example, Parkinson's disease, Alzheimer's disease and amyotrophic lateral sclerosis (ALS).

### BACKGROUND OF THE INVENTION

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology, but, in the last 15 years, there has been an important development of the understanding of the genetic basis of this disease and associated pathogenic mechanisms. One area of the development is the understanding of leucine rich repeat kinase 2 (LRRK2) protein. A number of mis-sense mutations in the LRRK2 gene have been strongly linked with autosomal dominant Parkinson's disease in familial studies (See WO2006068492 and WO2006045392; Trinh and Farrer 2013, Nature Reviews in Neurology 9: 445-454; Paisan-Ruiz et al., 2013, J. Parkinson's Disease 3: 85-103). The G2019S mutation in LRRK2 is the most frequent mis-sense mutation and is associated with a clinical phenotype that closely resembles sporadic Parkinson's disease. The LRRK2 G2019S mutation is also present in approximately 1.5% of sporadic Parkinson's disease cases (See Gilks et al., 2005, Lancet, 365: 415-416). In addition to the known pathogenic coding mutations in LRRK2, additional amino acid coding variants of LRRK2 have been identified that are also associated with risk of developing Parkinson's disease (See Ross et al., 2011 Lancet Neurology 10: 898-908). Furthermore, genome-wide association studies (GWAS) have identified LRRK2 as a Parkinson's disease susceptibility locus, which indicates that LRRK2 may be also relevant to sporadic Parkinson's disease cases without mutations that cause amino acid substitutions in the LRRK2 protein. (See Satake et al., 2009 Nature Genetics 41:1303-1307; Simon-Sanchez et al 2009 Nature Genetics 41: 1308-1312)

LRRK2 is a member of the ROCO protein family and all members of this family share five conserved domains. The most common pathogenic mutation G2019S occurs in the highly conserved kinase domain of LRRK2. This mutation confers an increase in the LRRK2 kinase activity in in vitro enzyme assays of recombinant LRRK2 proteins (See Jaleel et al., 2007, Biochem J, 405: 307-317) and in LRRK2 proteins purified from G2019S PD patient-derived cells (See Dzamko et al., 2010 Biochem. J. 430: 405-413). A less frequent LRRK2 pathogenic mutation that confers amino acid substitution at a different residue, R1441, has also been shown to elevate LRRK2 kinase activity by decreasing the rate of GTP hydrolysis by the GTPase domain of LRRK2 (See Guo et al., 2007 Exp Cell Res. 313: 3658-3670; West et al., 2007 Hum. Mol Gen. 16: 223-232). Moreover, phosphorylation of Rab protein physiologic substrates of LRRK2 has been shown to be increased by a range of Parkinson's disease pathogenic mutations of LRRK2 (See Steger et al., 2016 eLife 5 e12813). Therefore, the evidence indicates that the kinase and GTPase activities of LRRK2 are important for pathogenesis, and that the LRRK2 kinase domain may regulate overall LRRK2 function (See Cookson, 2010 Nat. Rev. Neurosci. 11: 791-797).

There is evidence to show that the increased LRRK2 kinase activity is associated with neuronal toxicity in cell culture models (See Smith et al., 2006 Nature Neuroscience 9: 1231-1233) and kinase inhibitor compounds protect against LRRK2-mediated cell death (See Lee et al., 2010 Nat. Med. 16: 998-1000). LRRK2 has also been reported to act as a negative regulator of microglial-mediated clearance of alpha-synuclein (See Maekawa et al., 2016 BMC Neuroscience 17:77), suggesting a possible utility of LRRK2 inhibitors in promoting clearance of neurotoxic forms of alpha-synuclein in the treatment of Parkinson's disease.

Induced pluripotent stem cells (iPSCs) derived from LRRK2 G2019S Parkinson's disease patients have been found to exhibit defects in neurite outgrowth and increased susceptibility to rotenone, that may be ameliorated by either genetic correction of the G2019S mutation or treatment of cells with small molecule inhibitors of LRRK2 kinase activity (See Reinhardt et al., 2013 Cell Stem Cell 12: 354-367). Mitochondrial DNA damage has been reported as a molecular marker of vulnerable dopamine neurons in substantia nigra of postmortem Parkinson's disease specimens (See Sanders et al 2014 Neurobiol. Dis. 70: 214-223). Increased levels of such mitochondrial DNA damage associated with LRRK2 G2019S mutation in iSPCs is blocked by genetic correction of the G2019S mutation (See Sanders et al., 2014 Neurobiol. Dis. 62: 381-386).

Additional evidence links LRRK2 function and dysfunction with autophagy-lysosomal pathways (See Manzoni and Lewis, 2013 Faseb J. 27:3234-3429). LRRK2 proteins confer defects in chaperone-mediated autophagy that negatively impact the ability of cells to degrade alpha-synuclein (Orenstein et al., 2013 Nature Neurosci. 16 394-406). In other cell models, selective LRRK2 inhibitors have been shown to stimulate macroautophagy (See Manzoni et al., 2013 BBA Mol. Cell Res. 1833: 2900-2910). These data suggest that small molecule inhibitors of LRRK2 kinase activity may have utility in the treatment of diseases characterized by defects in cellular proteostasis that result from aberrant autophagy/lysosomal degradation pathways including forms of Parkinson's disease associated with GBA mutations (See Swan and Saunders-Pullman 2013 Curr. Neurol. Neurosci Rep. 13: 368), other alpha-synucleinopathies, tauopathies, Alzheimer's disease (See Li et al., 2010 Neurodegen. Dis. 7: 265-271) and other neurodegenerative diseases (See Nixon 2013 Nat. Med. 19: 983-997) and Gaucher disease (See Westbroek et al., 2011 Trends. Mol. Med. 17: 485-493). As promoters of autophagy, small molecule inhibitors of LRRK2 kinase may also have utility in the treatment of other diseases including diabetes, obesity, motor neuron disease, epilepsy and some cancers (See Rubinsztein et al., 2012 Nat. Rev. Drug Discovery 11: 709-730), pulmonary diseases such as chronic obstructive pulmonary disease and idiopathic pulmonary fibrosis (See Araya et al., 2013 Intern. Med. 52: 2295-2303) and autoimmune diseases such as systemic lupus erythematosus (See Martinez et al., 2016 Nature 533: 115-119). As promoters of autophagy and phagocytic processes, small molecule inhibitors of LRRK2 kinase may also have utility in augmenting host responses in the treatment of a range of intracellular bacterial infections, parasitic infections and viral infections, including diseases such as tuberculosis (See Rubinsztein et al., 2012 Nat. Rev. Drug Discovery 11: 709-730; Araya et al., 2013 Intern. Med. 52: 2295-2303; Gutierrez, Biochemical Society Conference; Leucine rich repeat kinase 2: ten years along the road to therapeutic intervention, Henley Business School, UK 12 Jul. 2016), HIV, West Nile Virus and chikungunya virus (see Shoji-Kawata et al., 2013 Nature 494: 201-206). LRRK2 inhibitors may have utility in the treatment of such diseases alone, or in combination with drugs that directly target the infectious agent. Further, significantly elevated levels of LRRK2 mRNA have also been observed in fibroblasts of Niemann-Pick Type C (NPC) disease patients compared with fibroblasts of normal subjects, which indicates that aberrant LRRK2 function may play a role in lysosomal disorders (See Reddy et al., 2006 PLOS One 1 (1):e19 doi: 10.1371/journal.pone.0000019—supporting information Dataset 51). This observation suggests that LRRK2 inhibitors may have utility for the treatment of NPC.

The PD-associated G2019S mutant form of LRRK2 has also been reported to enhance phosphorylation of tubulin-associated Tau (See Kawakami et al., 2012 PLoS ONE 7: e30834, doi 10.1371), and disease models have been proposed in which LRRK2 acts upstream of the pathogenic effects of Tau and alpha-synuclein (See Taymans & Cookson, 2010, BioEssays 32: 227-235). In support of this, LRRK2 expression has been associated with increased aggregation of insoluble Tau, and increased Tau phosphorylation, in a transgenic mouse model (See Bailey et al., 2013 Acta Neuropath. 126:809-827). Overexpression of the PD pathogenic mutant protein LRRK2 R1441G is reported to cause symptoms of Parkinson's disease and hyperphosphorylation of Tau in transgenic mouse models (See Li, Y. et al. 2009, Nature Neuroscience 12: 826-828). Therefore, these data suggest that LRRK2 inhibitors of kinase catalytic activity may be useful for the treatment of tauopathy diseases characterized by hyperphosphorylation of Tau such as argyrophilic grain disease, Pick's disease, corticobasal degeneration, progressive supranuclear palsy and inherited frontotemporal dementia and parkinsonism linked to chromosome 17 (FTDP-17) (See Goedert, M and Jakes, R (2005) Biochemica et Biophysica Acta 1739, 240-250). In addition, LRRK2 inhibitors may have utility in the treatment of other diseases characterized by diminished dopamine levels such as withdrawal symptoms/relapse associated with drug addiction (See Rothman et al., 2008, Prog. Brain Res, 172: 385).

Other studies have also shown that overexpression of the G2019S mutant form of LRRK2 confers defects in subventricular zone (SVZ) neuroprogenitor cell proliferation and migration in transgenic mouse models (See Winner et al., 2011 Neurobiol. Dis. 41: 706-716) and reduces neurite length and branching cell culture models (See Dachsel et al., 2010 Parkinsonism & Related Disorders 16: 650-655). Moreover, it was reported that agents that promote SVZ neuroprogenitor cell proliferation and migration also improve neurological outcomes following ischemic injury in rodent models of stroke (See Zhang et al., 2010 J. Neurosci. Res. 88: 3275-3281). These findings suggest that compounds that inhibit aberrant activity of LRRK2 may have utility for the treatments designed to stimulate restoration of CNS functions following neuronal injury, such as ischemic stroke, traumatic brain injury, spinal cord injury.

Mutations in LRRK2 have also been identified that are clinically associated with the transition from mild cognitive impairment (MCI) to Alzheimer's disease (See WO2007149798). These data suggest that inhibitors of LRRK2 kinase activity may be useful for the treatment diseases such as Alzheimer's disease, other dementias and related neurodegenerative disorders.

Aberrant regulation of normal LRRK2 proteins is also observed in some disease tissues and models of disease. Normal mechanisms of translational control of LRRK2 by miR-205 are perturbed in some sporadic PD cases, where significant decreases in miR-205 levels in PD brain samples concur with elevated LRRK2 protein levels in those samples (See Cho et al., (2013) Hum. Mol. Gen. 22: 608-620). Therefore, LRRK2 inhibitors may be used in the treatment of sporadic PD patients who have elevated levels of normal LRRK2 proteins.

In an experimental model of Parkinson's disease in marmosets, an elevation of LRRK2 mRNA is observed in a manner that correlates with the level of L-Dopa induced dyskinesia (See Hurley, M. J et al., 2007 Eur. J. Neurosci. 26: 171-177). This suggests that LRRK2 inhibitors may have a utility in amelioration of such dyskinesias.

Significantly elevated levels of LRRK2 mRNA have been reported in ALS patient muscle biopsy samples (See Shtilbans et al., 2011 Amyotrophic Lateral Sclerosis 12: 250-256) It is suggested that elevated levels of LRRK2 kinase activity may be a characteristic feature of ALS. Therefore, this observation indicated that LRRK2 inhibitor may have utility for the treatment of ALS.

There is also evidence indicating that LRRK2 kinase activity may play a role in mediating microglial proinflammatory responses (See Moehle et al., 2012, J. Neuroscience 32: 1602-1611). This observation suggests a possible utility of LRRK2 inhibitors for the treatment of aberrant neuroinflammatory mechanisms that contribute to a range of neurodegenerative diseases, including Parkinson's disease, Alzheimer's disease, multiple sclerosis, HIV-induced dementia, amyotrophic lateral sclerosis, ischemic stroke, traumatic brain injury and spinal cord injury. Some evidence also indicates that LRRK2 plays a role in regulating neuronal progenitor differentiation in vitro (See Milosevic, J. et al., 2009 Mol. Neurodegen. 4: 25). This evidence suggests that inhibitors of LRRK2 may have a utility in production of neuronal progenitor cells in vitro for consequent therapeutic application in cell based-treatment of CNS disorders.

It has been reported that Parkinson's disease patients bearing LRRK2 G2019S mutation display increased frequency of non-skin cancers, including renal, breast, lung and prostate cancers, as well as acute myelogenous leukemia (AML). Since there is evidence to show that G2019S mutation in LRRK2 increases catalytic activity of the LRRK2 kinase domain, small molecule inhibitors of LRRK2 may have a utility in the treatment of cancers, for example kidney cancer, breast cancer, lung cancer, prostate cancer (e.g. solid tumors) and blood cancer (See. AML; Saunders-Pullman et al., 2010, Movement Disorders, 25:2536-2541; Inzelberg et al., 2012 Neurology 78: 781-786). Amplification and over-expression of LRRK2 has also been reported in papillary renal and thyroid carcinomas, where co-operativity between LRRK2 and the MET oncogene may promote tumor cell growth and survival (See Looyenga et al., 2011 PNAS 108: 1439-1444.)

Some studies have suggested that there is a genetic association of common LRRK2 variants with susceptibility to ankylosing spondylitis (See Danoy P, et al., 2010. PLoS Genet.; 6(12):e1001195; and leprosy infection (See Zhang F R, et al. 2009, N Engl J Med. 361:2609-18.). These findings suggest that inhibitors of LRRK2 may have utility in the treatment of ankylosing spondylitis and leprosy infection.

Meta-analysis of three genome wide associated scans for Crohn's disease identified a number of loci associated with the disease, including the locus containing the LRRK2 gene (See Barrett et al., 2008, Nature Genetics, 40: 955-962). Evidence has also emerged that LRRK2 is an IFN-γ target gene that may be involved in signaling pathways relevant to Crohn's disease pathogenesis (See Gardet et al., 2010, J. Immunology, 185: 5577-5585). These findings suggest that inhibitors of LRRK2 may have utility in the treatment of Crohn's disease.

As an IFN-γ target gene, LRRK2 may also play a role in T cell mechanisms that underlie other diseases of the immune system such as multiple sclerosis and rheumatoid arthritis. Further potential utility of LRRK2 inhibitors comes from the reported finding that B lymphocytes constitute a major population of LRRK2 expressing cells (See Maekawa et al. 2010, BBRC 392: 431-435). This suggests that LRRK2 inhibitors may be effective in the treatment of diseases of the immune system for which B cell depletion is, or may be, effective in diseases such as lymphomas, leukemias, multiple sclerosis (See Ray et al., 2011 J. Immunol. 230: 109), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, Sjögren's syndrome, Devic's disease and inflammatory myopathies (See Engel et al., 2011 Pharmacol. Rev. 63: 127-156; Homam et al., 2010 J. Clin. Neuromuscular Disease 12: 91-102).

### SUMMARY OF THE INVENTION

The present invention provides, in a first aspect, a compound of Formula (I)

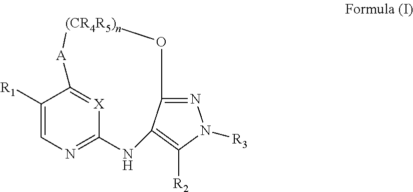

or a pharmaceutically acceptable salt thereof, wherein
X is CH or N;
n is 2, 3, 4 or 5;
A is O or NR^{a}, wherein
   R^{a} is
      H;
      C₁₋₄alkyl optionally substituted with one to three substituents independently selected from the group consisting of halo, hydroxyl and C₁₋₃alkoxyl, wherein C₁₋₃alkoxyl is optionally substituted with one to three halo substituents;
      C₃₋₆cycloalkyl optionally substituted with one to three substituents independently selected from the group consisting of halo, hydroxyl and C₁₋₃alkoxyl; or
      four to seven-membered heterocyclyl ring having one or two heteroatom ring members independently selected from O and N, and the heterocyclyl ring is optionally substituted with one to three substituents independently selected from halo and C₁₋₃alkyl, which C₁₋₃alkyl is optionally substituted with one to three halo substituents;
R₁ is
   1) H, halo, CN;
   2) C₁₋₄alkyl optionally substituted with one to three substituents independently selected from the group consisting of halo, hydroxyl and C₁₋₃alkoxyl, which C₁₋₃alkoxyl is optionally substituted with one to three halo substituents;
   3) C₂₋₆ alkenyl optionally substituted with one to three halo or C₁₋₃alkyl, which C₁₋₃alkyl is optionally substituted with one to three halo substituents;
   4) C₂₋₆ alkynyl optionally substituted with one to three C₁₋₃alkyl substituents, which C₁₋₃alkyl is optionally substituted with one to three halo substituents;
   5) C₁₋₄alkoxyl optionally substituted with one to three halo substituents;
   6) C₃₋₆cycloalkyl optionally substituted with one to three substituents independently selected from the group consisting of halo, hydroxyl, C₁₋₃alkoxyl and C₁₋₃alkyl, wherein C₁₋₃alkoxyl and C₁₋₃alkyl are optionally substituted with one to three halo substituents;
   7) —OC₃₋₆cycloalkyl optionally substituted with one to three substituents independently selected from the group consisting of halo, hydroxyl, C₁₋₃alkoxyl and C₁₋₃alkyl;
   8) four to seven-membered heterocyclyl ring having one or two heteroatom ring members independently selected from O and N, and the heterocyclyl ring is optionally substituted with one to three substituents independently selected from the group consisting of halo, hydroxyl, C₁₋₃alkoxyl and C₁₋₃alkyl;
   9) —O-heterocyclyl ring having one or two heteroatom ring members independently selected from O and N, wherein the heterocyclyl ring is a four to seven-membered ring optionally substituted with one to three substituents independently selected from the group consisting of halo, hydroxyl, C₁₋₃alkoxyl and C₁₋₃alkyl; or
   10) —SC₁₋₄alkyl optionally substituted with one to three halo substituents;
R₂ is
   H, halo, CN;
   C₁₋₄alkoxyl optionally substituted with one to three halo substituents;
   C₁₋₄alkyl optionally substituted with one to three substituents independently selected from the group consisting of halo, hydroxyl and C₁₋₃alkoxyl, which C₁₋₃alkoxyl is optionally substituted with one to three halo substituents; or
   C₃₋₆cycloalkyl optionally substituted with one to three substituents independently selected from the group consisting of halo, hydroxyl and C₁₋₃alkoxyl;
R3 is
   1) H;
   2) —CO—Z, wherein Z is selected from the group consisting of
      four to seven-membered heterocyclyl ring having one or two heteroatom ring members independently selected from O and N;
      C₃₋₆cycloalkyl optionally substituted with one to three substituents independently selected from the group consisting of halo, hydroxyl, C₁₋₃alkyl, and C₁₋₃alkoxyl; and
      C₁₋₆alkyl optionally substituted with one to three substituents independently selected from halo and C₁₋₃alkoxyl;
   3) four to seven-membered heterocyclyl ring having one or two heteroatom ring members independently selected from O and N, and the heterocyclyl ring is optionally substituted with one to three substituents independently selected from the group consisting of
      halo;
      cyano;
      C₁₋₃alkyl optionally substituted with one to three substituents independently selected from halo, hydroxyl and C₁₋₃alkoxyl;
      C₁₋₃alkoxyl optionally substituted with one to three substituents independently selected from halo, hydroxyl and C₁₋₃alkoxyl; and
      four to seven-membered heterocyclyl ring having one or two heteroatom ring members independently selected from O and N, and the heterocyclyl ring is optionally substituted with one to three substituents independently selected from the group consisting of halo, C₁₋₃alkyl and C₁₋₃alkoxyl;
   4) C₁₋₆alkyl optionally substituted with one to three substituents independently selected from the group consisting of
      CN, hydroxyl, halo;
      C₁₋₄alkoxyl optionally substituted with one to three substituents independently selected from halo, hydroxyl and C₁₋₃alkoxyl;
      —CO-Q, wherein Q is C₁₋₄alkoxyl, hydroxylor NR_{c}R_{d}, wherein R_{c} and R_{d} are each independently H or C₁₋₄alkyl;

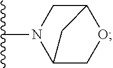 [SMILES: *N1CC2CC1CO2]

and
four to seven-membered heterocyclyl ring having one or two heteroatom ring members independently selected from O and N, wherein the heterocyclyl ring is optionally substituted with one to three halo substituents;
5) C₃₋₇cycloalkyl optionally substituted with one to three substituents independently selected from the group consisting of
   halo, hydroxyl;

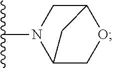 [SMILES: *N1CC2CC1CO2]

C₁₋₃alkyl optionally substituted with one to three substituents independently selected from the group consisting of halo, hydroxyl and C₁₋₃alkoxyl;
C₁₋₄alkoxyl optionally substituted with one to three substituents independently selected from the group consisting of halo, hydroxyl and C₁₋₃alkoxyl; and
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl;
6) C-linked 7-9 membered bridged cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
7) C-linked 7-10 membered spirane cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl; or
8) C-linked 6-9 membered fused cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
R4 and R5, at each occurrence, are each independently selected from the group consisting of
   H, halo, hydroxyl;
   C₁₋₄alkyl optionally substituted with one to three substituents independently selected from the group consisting of halo, C₁₋₄alkoxyl, OC₁₋₄haloalkyl, and four to seven-membered heterocyclyl ring having one or two heteroatom ring members independently selected from O and N;
   C₃₋₆cycloalkyl optionally substituted with one to three substituents independently selected from the group consisting of halo, hydroxyl and C₁₋₄alkoxy;
   four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl; and
   C₁₋₄alkoxyl optionally substituted with one to three substituents independently selected from the group consisting of halo, hydroxyl and C₁₋₄alkoxyl.

In a further aspect of the invention, the invention provides a pharmaceutical composition comprising a compound of Formula (I) or a pharmaceutically acceptable salt thereof and a pharmaceutically acceptable excipient.

A further aspect of the invention provides a compound of Formula (I) or a pharmaceutically acceptable salt thereof for use in the treatment or prevention of Parkinson's disease, Alzheimer's disease, and amyotrophic lateral sclerosis (ALS).

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, the nomenclature used herein and the laboratory procedures in organic chemistry, medicinal chemistry, biology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

### A. Definitions

As used herein, “alkyl” refers to a monovalent, saturated hydrocarbon chain having a specified number of carbon atoms. For example, C₁₋₄ alkyl refers to an alkyl group having from 1 to 4 carbon atoms. Alkyl groups may be straight or branched. In some embodiments, branched alkyl groups may have one to three branches. Examples of alkyl groups include, but are not limited to, methyl, ethyl, propyl (n-propyl and isopropyl), butyl, pentyl and hexyl. As used herein, “—SC₁₋₄ alkyl” refers to a C₁₋₄ alkyl group that is linked to the core through a sulfur (S) atom.

As used herein, “alkoxyl” refers to the group —O-alkyl. For example, C₁₋₆ alkoxyl groups contain from 1 to 6 carbon atoms. C₁₋₄ alkoxyl groups contain from 1 to 4 carbon atoms. C₁₋₃ alkoxyl groups contain from 1 to 3 carbon atoms. Examples of alkoxyl groups include, but are not limited to, methoxyl, ethoxyl, propoxyl, butoxyl, pentyloxyl, and hexyloxyl.

As used herein, “cycloalkyl” refers to a saturated monocyclic hydrocarbon ring having a specified number of carbon atoms. For example, C₃₋₆ cycloalkyl contains 3 to 6 carbon atoms as member atoms in the ring. —OC₃₋₆ cycloalkyl refers to a C₃₋₆ cycloalkyl group that is linked to the core through an oxygen atom. Examples of C₃₋₆ cycloalkyl include, but are not limited to, cyclobutyl, cyclopentyl and cyclohexyl.

As used herein, “alkenyl” refers to a straight or branched hydrocarbon chain having a specified number of carbon atoms, containing at least one double bond. For example, C₂₋₆ alkenyl refers to an alkenyl group having from 2 to 6 carbon atoms. Exemplary alkenyl groups include, but are not limited to, ethenyl (i.e., vinyl), prop-1-enyl (i.e., allyl), but-1-enyl, pent-1-enyl and hexenyl.

As used herein, “alkynyl” refers to a straight or branched hydrocarbon chain having a specified number of carbon atoms, containing at least one triple bond. For example, C₂₋₆ alkynyl refers to an alkynyl group having from 2 to 6 carbon atoms. Examples of alkynyl groups include, but are not limited to, ethynyl, propynyl, butynyl, pentynyl and hexynyl.

As used herein, “halogen” refers to fluorine (F), chlorine (Cl), bromine (Br), or iodine (I). “Halo” refers to the halogen radicals: fluoro (—F), chloro (—Cl), bromo (—Br), or iodo (—I).

As used herein, “haloalkyl” refers to an alkyl group, as defined above, substituted with one or more halogen atoms independently selected from F, Cl, Br, or I, on any or all of the carbon atoms of the alkyl group. For example, C₁₋₄haloalkyl refers to a C₁₋₄alkyl group substituted with one or more halogen atoms. —OC₁₋₄haloalky refers to a C₁₋₄haloalkyl group that is linked through an oxygen atom. Exemplary haloalkyl groups include, but are not limited to, fluoromethyl, chloromethyl, bromoethyl, difluoromethyl, trifluoromethyl, and dichloromethyl.

As used herein, “substituted” in reference to a group indicates that one or more hydrogen atom(s) attached to a member atom (e.g., carbon atom) within the group is replaced with a substituent selected from the group of defined substituents. It should be understood that the term “substituted” includes the implicit provision that such substitution is in accordance with the permitted valence of the substituted atom and the substituent and that the substitution results in a stable compound (i.e. one that does not spontaneously undergo transformation such as by rearrangement, cyclization, or elimination and that is sufficiently robust to survive isolation from a reaction mixture). When it is stated that a group may contain one or more substituent(s), one or more (as appropriate) member atom(s) within the group may be substituted. In addition, a single member atom within the group may be substituted with more than one substituent as long as such substitution is in accordance with the permitted valence of the atom.

As used herein, “optionally substituted” indicates that a particular group, such as alkyl, alkenyl, alkynyl, alkoxyl, heterocyclyl, cycloalkyl,

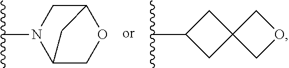 [SMILES: *N1CC2CC1CO2 ; "or" ; *C1CC2(COC2)C1]

may be unsubstituted, or may be substituted as further defined.

As used herein, “heterocyclyl” or “heterocyclyl ring” is a monovalent radical derived by removal of a hydrogen atom from a saturated monocyclic ring, which ring consists of ring carbon atoms and 1 or 2 ring heteroatoms independently selected from nitrogen and oxygen. The number of ring atoms may be specified. For example, a “four to seven-membered heterocyclyl,” or “four to seven-membered heterocyclyl ring,” is a heterocyclyl or heterocyclyl ring as defined above consisting of four to seven ring atoms. Other ring heteroatoms (nitrogen or oxygen) may additionally be present. A heterocyclyl ring may be linked to the core thorough an atom that is not part of the ring. For example, an “O-heterocyclyl” or “O-heterocyclyl ring” is a heterocyclyl or herterocyclyl ring as defined above that is linked to the core through an oxygen atom. In one embodiment, the heterocyclyl ring is attached directly to the core. Examples of heterocyclyl rings include, but are not limited to, oxetanyl, azetidinyl, tetrahydrofuranyl (including, for example, tetrahydrofuran-2-yl and tetrahydrofuran-3-yl), pyrrolidinyl (including, for example, pyrrolidin-1-yl and pyrrolidin-3-yl), tetrahydro-2H-pyranyl or oxanyl (including, for example, tetrahydro-2H-pyran-3-yl or oxayn-3-yl and tetrahydro-2H-pyran-4-yl or oxan-4-yl), piperidinyl (including, for example, piperidin-3-yl and piperidin-4-yl) and morpholinyl (including, for example, morpholin-2-yl and morpholin-4-yl).

As used herein, the term “oxygen-containing heterocyclyl” or “oxygen-containing heterocyclyl ring” is a monovalent radical derived by removal of a hydrogen atom from a saturated monocyclic ring, which ring consists of ring carbon atoms and 1 oxygen atom. The number of ring atoms may be specified. For example, a “four to seven-membered oxygen-containing heterocyclyl,” or “four to seven-membered oxygen-containing heterocyclyl ring,” is an oxygen containing heterocyclyl or oxygen containing heterocyclyl ring as defined above consisting of four to seven ring atoms. Examples of oxygen-containing heterocyclyl rings include, but are not limited to, oxetanyl, tetrahydrofuranyl (including, for example, tetrahydrofuran-2-yl and tetrahydrofuran-3-yl), tetrahydro-2H-pyranyl or oxanyl (including, for example, tetrahydro-2H-pyran-3-yl or oxayn-3-yl and tetrahydro-2H-pyran-4-yl or oxan-4-yl),

As used herein, the term “nitrogen-containing heterocyclyl” or “nitrogen-containing heterocyclyl ring” is a monovalent radical derived by removal of a hydrogen atom from a saturated monocyclic ring, which ring consists of ring carbon atoms and 1 nitrogen atom. The number of ring atoms may be specified. For example, a “four to seven-membered nitrogen-containing heterocyclyl,” or “four to seven-membered nitrogen-containing heterocyclyl ring,” is a nitrogen-containing heterocyclyl or nitrogen-containing heterocyclyl ring as defined above consisting of four to seven ring atoms. Examples of nitrogen-containing heterocyclyl rings include, but are not limited to azetidinyl, pyrrolidinyl (including, for example, pyrrolidin-1-yl and pyrrolidin-3-yl) and piperidinyl (including, for example, piperidin-3-yl and piperidin-4-yl).

As used herein, the term “bridged cyclyl ring” refers to a monovalent radical derived by removal of a hydrogen atom from a bridged ring (a ring in which two non adjacent ring atoms are linked by a bridge containing at least one atom), which consists of carbon atoms and 0 to 2 heteroatoms independently selected from nitrogen and oxygen. In one embodiment, the bridged cyclyl ring is saturated. The number of atoms in the ring and bridge may be specified. For example, the term 7-9 membered bridged cyclyl ring refers to a bridged cyclyl ring that has a total of 7 to 9 atoms in the ring plus bridge. The term “C-linked 7-9 membered bridged cyclyl ring” refers to a 7-9 membered bridged cyclyl ring as defined above that contains one carbon ring atom through which it is linked to the core. Examples of bridged cyclic rings include, but are not limited to,

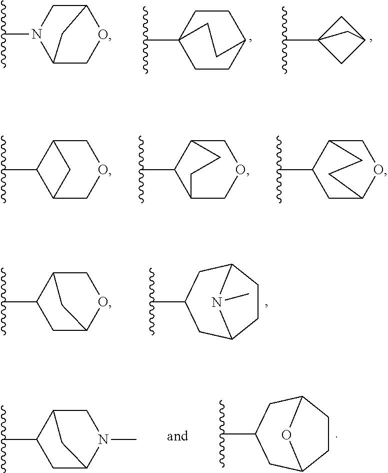

As used herein, the term “spirane cyclyl ring” refers to a monovalent radical derived by removal of a hydrogen atom from two rings connected through just one atom. The spirane cyclyl ring consists of carbon atoms and 0 to 2 heteroatoms independently selected from nitrogen and oxygen but the spiro atom must be carbon. In one embodiment, the spirane cyclyl ring is saturated. The number of atoms in the spirane cyclyl ring may be specified. For example, a 7-10 membered spirane cyclyl ring refers to a spirane cyclyl ring that has a total of 7 to 10 atoms in the two rings (including the spiro-atom). The term “C-linked 7-10 membered spirane cyclyl ring” refers to a 7-10 membered spirane cyclyl ring as defined above that contains one carbon ring atom through which it is linked to the core. Examples of spirane cyclyl rings and C-linked spirane cyclyl rings include, but are not limited to,

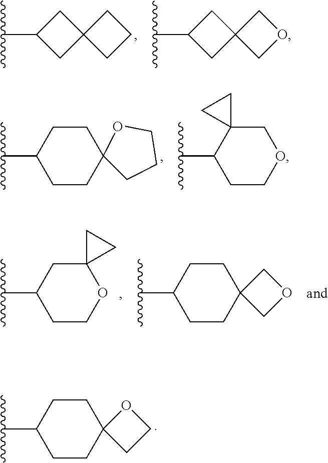

As used herein, the term “fused cyclyl ring” refers to a monovalent radical derived by removal of a hydrogen atom from a bicyclic ring (two rings sharing one bond), which consists of carbon atoms and 0 to 2 heteroatoms independently selected from nitrogen and oxygen. The number of atoms in a fused cyclyl ring may be specified. In one embodiment, the fused cyclyl ring is saturated. For example, the term 6-9 membered fused cyclyl ring refers to a fused bicyclic ring that has a total of 6 to 9 atoms in the two rings. The term “C-linked 6-9 membered fused cyclyl ring” refers to a 6-9 membered fused bicyclic ring as defined above that contains at least one carbon ring atom through which it is linked to the core. One or two other ring heteroatoms (nitrogen or oxygen) may additionally be present. Examples of fused cyclyl rings include, but are not limited to

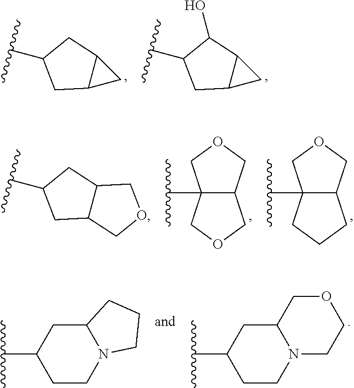

As used herein, the structure

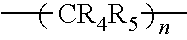

or (CR₄R₅)ₙ refers to

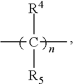

as would be understood by a skilled artisan.

As used herein, the term “leaving group” means the group with the meaning conventionally associated with it in synthetic organic chemistry, i.e., an atom or group displaceable under substitution reaction conditions. Examples of leaving groups include, but are not limited to, methanesulfonate and 4-methylbenzenesulfonate.

As used herein, the term “protecting group” or “protective group” means the group with the meaning conventionally associated with it in synthetic chemistry, i.e., a group that selectively blocks one reactive site in a compound that has multiple reactive sites such that a chemical reaction can be carried out selectively at another unprotected reactive site. Some processes of this invention rely upon the protective groups to block reactive nitrogen and/or oxygen atoms present in the reactants. Examples of protective groups include, but are not limited to tetrahydro-2H-pyran, Boc(tert-butyloxycarbonyl) or ((trimethylsilyl)ethoxy)methyl).

As used herein, the term “disease” refers to any alteration in state of the body or of some of the organs, interrupting or disturbing the performance of the functions and/or causing symptoms such as discomfort, dysfunction, distress, or even death to the person afflicted. A disease can also include a distemper, ailing, ailment, malady, disorder, sickness, illness, complain, interdisposition and/or affectation.

As used herein, “treat”, “treating” or “treatment” in reference to a disease means: (1) to ameliorate the disease or one or more of the biological manifestations of the disease, (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease. Symptomatic treatment refers to treatment as referred to in point (1), (3) and (5). Disease modifying treatment refers to treatment as defined in point (2) and (4).

As used herein, “prevent”, “preventing” or “prevention” means the prophylactic administration of a drug to diminish the likelihood of the onset of or to delay the onset of a disease or biological manifestation thereof.

As used herein, “subject” means a mammalian subject (e.g., dog, cat, horse, cow, sheep, goat, monkey, etc.), and human subjects including both male and female subjects, and including neonatal, infant, juvenile, adolescent, adult and geriatric subjects, and further including various races and ethnicities including, but are not limited to, white, black, Asian, American Indian and Hispanic.

As used herein, “pharmaceutically acceptable salt(s)” refers to salt(s) that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, “therapeutically effective amount” in reference to a compound of the invention means an amount of the compound sufficient to treat or prevent the patient's disease but low enough to avoid serious side effects (at a reasonable benefit/risk ratio) within the scope of sound medical judgment. A therapeutically effective amount of a compound will vary with the particular compound chosen (e.g. consider the potency, efficacy, and half-life of the compound); the route of administration chosen; the disease being treated; the severity of the disease being treated; the age, size, weight, and physical disease of the patient being treated; the medical history of the patient to be treated; the duration of the treatment; the nature of concurrent therapy; the desired therapeutic effect; and like factors, but can nevertheless be routinely determined by the skilled artisans.

### B. Compounds

This invention provides, in a first aspect, a compound of Formula (I):

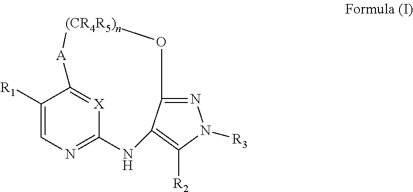

or a pharmaceutically acceptable salt thereof, wherein
X is CH or N;
n is 2, 3, 4 or 5;
A is O or NR^{a}, wherein
   R^{a} is
      H;
      C₁₋₄alkyl optionally substituted with one to three substituents independently selected from the group consisting of halo, hydroxyl and C₁₋₃alkoxyl, wherein C₁₋₃alkoxyl is optionally substituted with one to three halo substituents;
      C₃₋₆cycloalkyl optionally substituted with one to three substituents independently selected from the group consisting of halo, hydroxyl and C₁₋₃alkoxyl; or
      four to seven-membered heterocyclyl ring having one or two heteroatom ring members independently selected from O and N, and the heterocyclyl ring is optionally substituted with one to three substituents independently selected from halo and C₁₋₃alkyl, which C₁₋₃alkyl is optionally substituted with one to three halo substituents;
R₁ is
   1) H, halo, CN;
   2) C₁₋₄alkyl optionally substituted with one to three substituents independently selected from the group consisting of halo, hydroxyl and C₁₋₃alkoxyl, which C₁₋₃alkoxyl is optionally substituted with one to three halo substituents;
   3) C₂₋₆ alkenyl optionally substituted with one to three halo or C₁₋₃alkyl, which C₁₋₃alkyl is optionally substituted with one to three halo substituents;
   4) C₂₋₆ alkynyl optionally substituted with one to three C₁₋₃alkyl substituents, which C₁₋₃alkyl is optionally substituted with one to three halo substituents;
   5) C₁₋₄alkoxyl optionally substituted with one to three halo substituents;
   6) C₃₋₆cycloalkyl optionally substituted with one to three substituents independently selected from the group consisting of halo, hydroxyl, C₁₋₃alkoxyl and C₁₋₃alkyl, wherein C₁₋₃alkoxyl and C₁₋₃alkyl are optionally substituted with one to three halo substituents;
   7) —OC₃₋₆cycloalkyl optionally substituted with one to three substituents independently selected from the group consisting of halo, hydroxyl, C₁₋₃alkoxyl and C₁₋₃alkyl;
   8) four to seven-membered heterocyclyl ring having one or two heteroatom ring members independently selected from O and N, and the heterocyclyl ring is optionally substituted with one to three substituents independently selected from the group consisting of halo, hydroxyl, C₁₋₃alkoxyl and C₁₋₃alkyl;
   9) —O-heterocyclyl ring having one or two heteroatom ring members independently selected from O and N, wherein the heterocyclyl ring is a four to seven-membered ring optionally substituted with one to three substituents independently selected from the group consisting of halo, hydroxyl, C₁₋₃alkoxyl and C₁₋₃alkyl; or
   10) —SC₁₋₄alkyl optionally substituted with one to three halo substituents;
R₂ is
   H, halo, CN;
   C₁₋₄alkoxyl optionally substituted with one to three halo substituents;
   C₁₋₄alkyl optionally substituted with one to three substituents independently selected from the group consisting of halo, hydroxyl and C₁₋₃alkoxyl, which C₁₋₃alkoxyl is optionally substituted with one to three halo substituents; or
   C₃₋₆cycloalkyl optionally substituted with one to three substituents independently selected from the group consisting of halo, hydroxyl and C₁₋₃alkoxyl;
R₃ is
   1) H;
   2) —CO—Z, wherein Z is selected from the group consisting of
      four to seven-membered heterocyclyl ring having one or two heteroatom ring members independently selected from O and N;
      C₃₋₆cycloalkyl optionally substituted with one to three substituents independently selected from the group consisting of halo, hydroxyl, C₁₋₃alkyl, and C₁₋₃alkoxyl; and
      C₁₋₆alkyl optionally substituted with one to three substituents independently selected from halo and C₁₋₃alkoxyl;
   3) four to seven-membered heterocyclyl ring having one or two heteroatom ring members independently selected from O and N, and the heterocyclyl ring is optionally substituted with one to three substituents independently selected from the group consisting of
      halo;
      cyano;
      C₁₋₃alkyl optionally substituted with one to three substituents independently selected from halo, hydroxyl and C₁₋₃alkoxyl;
      C₁₋₃alkoxyl optionally substituted with one to three substituents independently selected from halo, hydroxyl and C₁₋₃alkoxyl; and
      four to seven-membered heterocyclyl ring having one or two heteroatom ring members independently selected from O and N, and the heterocyclyl ring is optionally substituted with one to three substituents independently selected from the group consisting of halo, C₁₋₃alkyl and C₁₋₃alkoxyl;
   4) C₁₋₆alkyl optionally substituted with one to three substituents independently selected from the group consisting of
      CN, hydroxyl, halo;
      C₁₋₄alkoxyl optionally substituted with one to three substituents independently selected from halo, hydroxyl and C₁₋₃alkoxyl;
      —CO-Q, wherein Q is C₁₋₄alkoxyl, hydroxyl or NR_{c}R_{d}, wherein R_{c} and R_{d} are each independently H or C₁₋₄alkyl;

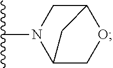 [SMILES: *N1CC2CC1CO2]

and
four to seven-membered heterocyclyl ring having one or two heteroatom ring members independently selected from O and N, wherein the heterocyclyl ring is optionally substituted with one to three halo substituents;
5) C₃₋₇cycloalkyl optionally substituted with one to three substituents independently selected from the group consisting of
   halo, hydroxyl;

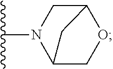 [SMILES: *N1CC2CC1CO2]

C₁₋₃alkyl optionally substituted with one to three substituents independently selected from the group consisting of halo, hydroxyl and C₁₋₃alkoxyl;
C₁₋₄alkoxyl optionally substituted with one to three substituents independently selected from the group consisting of halo, hydroxyl and C₁₋₃alkoxyl; and
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl;
6) C-linked 7-9 membered bridged cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
7) C-linked 7-10 membered spirane cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl; or
8) C-linked 6-9 membered fused cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
R₄ and R₅, at each occurrence, are each independently selected from the group consisting of
   H, halo, hydroxyl;
   C₁₋₄alkyl optionally substituted with one to three substituents independently selected from the group consisting of halo, C₁₋₄alkoxy, OC₁₋₄haloalkyl, and four to seven-membered heterocyclyl ring having one or two heteroatom ring members independently selected from O and N;
   C₃₋₆cycloalkyl optionally substituted with one to three substituents independently selected from the group consisting of halo, hydroxyl and C₁₋₄alkoxyl;
   four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl; and
   C₁₋₄alkoxyl optionally substituted with one to three substituents independently selected from the group consisting of halo, hydroxyl and C₁₋₄alkoxyl.

In certain embodiment, X is CH or N; n is 2, 3, 4 or 5; and A is O or NR^{a}, wherein R^{a} is H or C₁₋₄alkyl optionally substituted with C₁₋₃alkoxyl.

In one embodiment, A is NR^{a} and R^{a} is H, or C₁₋₄alkyl, which C₁₋₄alkyl group is optionally substituted with one to three substituents independently selected from the group consisting of halo, hydroxyl and C₁₋₃alkoxyl, wherein C₁₋₃alkoxyl is optionally substituted with one to three halo substituents.

In one embodiment, A is NR^{a} and R^{a} is H, or C₁₋₄alkyl which C₁₋₄alkyl group is optionally substituted with one substituents independently selected from the group consisting of halo, hydroxyl and C₁₋₃alkoxyl.

In one embodiment, A is NR^{a} and R^{a} is H, methyl or ethyl.

In one embodiment, A is NR^{a} and R^{a} is H.

In one embodiment, X is N.

In one embodiment, A is NR^{a}, R^{a} is H and X is N.

In one embodiment, R₁ is:
H, halo, CN;
C₁₋₄alkyl optionally substituted with one to three halo groups;
C₁₋₄alkoxyl optionally substituted with one to three halo groups;
C₂₋₆ alkenyl;
C₂₋₆ alkynyl; or
C₃₋₆cycloalkyl.

In one embodiment, R₁ is H, halo, CN, C₁₋₄alkoxyl, C₂₋₆ alkenyl, C₂₋₆ alkynyl or C₁₋₄alkyl optionally substituted with one to three substituents independently selected from the group consisting of halo, hydroxyl and C₁₋₃alkoxyl.

In certain embodiment, R₁ is selected from the group consisting of H, halo, CN, methyl, isopropyl, tert-butyl, methoxyl, trifluoromethyl, trifluoromethoxyl, ethenyl, prop-1-en-2-yl, ethynyl and cyclopropyl.

In certain embodiment, R₁ is selected from the group consisting of H, halo, CN, methyl, methoxy, trifluoromethyl, ethenyl and ethynyl.

In one embodiment, R₁ is selected from the group consisting of Br, Cl and CN.

In one embodiment, R₁ is Cl.

In one embodiment, R₂ is:
H, halo, CN;
C₁₋₄alkyl optionally substituted with one to three substituents independently selected from the group consisting of halo, hydroxyl and C₁₋₃alkoxyl, which C₁₋₃alkoxyl is optionally substituted with one to three halo substituents; or
C₃₋₆cycloalkyl optionally substituted with one to three substituents independently selected from the group consisting of halo, hydroxyl and C₁₋₃alkoxyl.

In one embodiment, R₂ is:
H, halo, CN;
C₁₋₄alkyl optionally substituted with one to three substituents independently selected from the group consisting of halo, hydroxyl and C₁₋₃alkoxyl; or
C₃₋₆cycloalkyl.

In certain embodiment, R₂ is H, halo, CN, C₁₋₄haloalkyl, C₁₋₄alkyl optionally substituted with C₁₋₃alkoxyl and C₃₋₆cycloalkyl.

In one embodiment, R₂ is selected from the group consisting of H, halo, CN, methyl, ethyl, difluoromethyl, trifluoromethyl, cyclopropyl, methoxymethyl and methoxyethyl (e.g. 1-methoxyethyl).

In one embodiment, R₂ is selected from the group consisting of H, halo, CN, methyl, difluoromethyl, trifluoromethyl, cyclopropyl and methoxyethyl (e.g. 1-methoxyethyl).

In one embodiment, R₂ is selected from the group consisting of Cl, CN and methyl.

In one embodiment, R₃ is
1) H;
2) —CO—Z, wherein Z is selected from the group consisting of
   four to seven-membered heterocyclyl ring having one or two heteroatom ring members independently selected from O and N;
   C₃₋₆cycloalkyl optionally substituted with one to three substituents independently selected from the group consisting of halo, hydroxyl, C₁₋₃alkyl, and C₁₋₃alkoxyl; and
   C₁₋₆alkyl optionally substituted with one to three substituents independently selected from halo and C₁₋₃alkoxyl;
3) four to seven-membered heterocyclyl ring having one or two heteroatom ring members independently selected from O and N, and the heterocyclyl ring is optionally substituted with one to three substituents independently selected from the group consisting of
   halo;
   C₁₋₃alkyl optionally substituted with one to three substituents independently selected from halo, hydroxyl and C₁₋₃alkoxyl;
   C₁₋₃alkoxyl optionally substituted with one to three substituents independently selected from halo, hydroxyl and C₁₋₃alkoxyl; and
   four to seven-membered heterocyclyl ring having one or two heteroatom ring members independently selected from O and N, and the heterocyclyl ring is optionally substituted with one to three substituents independently selected from the group consisting of halo, C₁₋₃alkyl and C₁₋₃alkoxyl;
4) C₁₋₆alkyl optionally substituted with one to three substituents independently selected from the group consisting of
   CN, hydroxyl, halo;
   C₁₋₄alkoxyl optionally substituted with one to three substituents independently selected from halo, hydroxyl and C₁₋₃alkoxyl;
   —CO-Q, wherein Q is C₁₋₄alkoxyl, hydroxyl, NH₂ or NR_{c}R_{d}, wherein R_{c} and R_{d} are independently H or C₁₋₄alkyl;

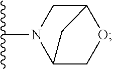 [SMILES: *N1CC2CC1CO2]

and
four to seven-membered heterocyclyl ring having one or two heteroatom ring members independently selected from O and N, wherein the heterocyclyl ring is optionally substituted with one to three halo substituents;
5) C₃₋₇cycloalkyl optionally substituted with one to three substituents independently selected from the group consisting of
   halo, hydroxyl;

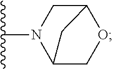 [SMILES: *N1CC2CC1CO2]

C₁₋₃alkyl optionally substituted with one to three substituents independently selected from the group consisting of halo, hydroxyl and C₁₋₃alkoxyl;
C₁₋₄alkoxyl optionally substituted with one to three substituents independently selected from the group consisting of halo, hydroxyl and C₁₋₃alkoxyl; and
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl;
6) C-linked 7-9 membered bridged cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
7) C-linked 7-10 membered spirane cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl; or
8) C-linked 6-9 membered fused cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;

In certain embodiment, R₃ is
1) H;
2) —CO—Z, wherein Z is selected from the group consisting of
   C₃₋₆cycloalkyl and four to six-membered heterocyclyl ring having 0 as heteroatom ring member;
3) four to six-membered heterocyclyl ring having one to two heteroatom ring members independently selected from O and N, and the heterocyclyl ring is optionally substituted with one to three substituents independently selected from the group consisting of
   halo;
   C₁₋₃alkyl optionally substituted with one to three substituents independently selected from halo, hydroxyl and C₁₋₃alkoxyl;
   C₁₋₃alkoxyl optionally substituted with one to three substituents independently selected from halo, hydroxyl and C₁₋₃alkoxyl; and
   four to six-membered heterocyclyl ring having one to two heteroatom ring members independently selected from O and N;
4) C₁₋₄alkyl optionally substituted with one to three substituents independently selected from the group consisting of
   CN, hydroxyl, halo;
   C₁₋₂alkoxyl;
   —CO-Q, wherein Q is C₁₋₂alkoxyl, hydroxyl, or NH₂;

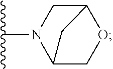 [SMILES: *N1CC2CC1CO2]

and
four to six-membered heterocyclyl ring having one to two heteroatom ring members independently selected from O and N, wherein the heterocyclyl ring is optionally substituted with one to three halo substituents;
5) C₃₋₆cycloalkyl optionally substituted with one to three substituents independently selected from the group consisting of
   halo, hydroxyl;

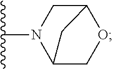 [SMILES: *N1CC2CC1CO2]

C₁₋₃alkyl optionally substituted with one to three substituents independently selected from halo, hydroxyl and C₁₋₃alkoxyl;
C₁₋₄alkoxyl optionally substituted with one to three substituents independently selected from halo, hydroxyl and C₁₋₃alkoxyl; and
four to six-membered heterocyclyl ring having one to two heteroatom ring members independently selected from O and N, wherein the heterocyclyl ring is optionally substituted with one to three halo substituents;
6) C-linked 7-9 membered bridged cyclyl ring optionally having one or two heteroatom ring members independently selected from O and N; or
7) C-linked 7-10 membered spirane cyclyl ring optionally having one or two heteroatom ring members independently selected from O and N.

In one embodiment, R₃ is
1) H;
2) —CO—Z, wherein Z is selected from the group consisting of cyclopropyl, oxetanyl and tetrahydro-2H-pyranyl;
3) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, wherein the heterocyclyl ring is optionally substituted with one to three substituents independently selected from the group consisting of
   hydroxyl, halo;
   C₁₋₃alkyl optionally substituted with one to three substituents independently selected from halo, hydroxyl and C₁₋₃alkoxyl;
   C₁₋₃alkoxyl optionally substituted with one to three substituents independently selected from halo, hydroxyl and C₁₋₃alkoxyl; and
   four to six-membered heterocyclyl ring having one to two heteroatom ring members independently selected from O and N;
4) C₁₋₄alkyl optionally substituted with one to three substituents independently selected from the group consisting of
   CN, hydroxyl, halo;
   C₁₋₂alkoxyl;
   —CO-Q, wherein Q is C₁₋₂alkoxyl, hydroxyl, or NH₂;

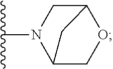 [SMILES: *N1CC2CC1CO2]

and
four to six-membered heterocyclyl ring selected from the group consisting of oxetanyl, tetrahydrofuranyl, tetrahydro-2H-pyranyl, pyrrolidinyl, piperidinyl and morpholinyl, wherein the heterocyclyl ring is optionally substituted with one to three halo substituents;
5) C₄₋₆ cycloalkyl optionally substituted with one to three substituents independently selected from the group consisting of
   halo, hydroxyl;

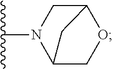 [SMILES: *N1CC2CC1CO2]

C₁₋₃alkyl optionally substituted with one to three substituents independently selected from halo, hydroxyl and C₁₋₃alkoxyl;
C₁₋₄alkoxyl optionally substituted with one to three substituents independently selected from halo, hydroxyl and C₁₋₃alkoxyl; and
four to six-membered heterocyclyl ring having one to two heteroatom ring members independently selected from O and N, wherein the heterocyclyl ring is optionally substituted with one to three halo substituents;

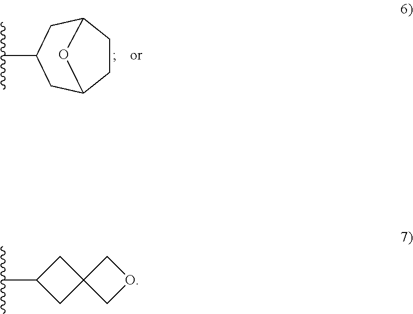 [SMILES: *C1CC2CCC(C1)O2 ; *C1CC2(COC2)C1]

In one embodiment, R₃ is H.

In one embodiment, R₃ is —CO—Z, wherein Z is selected from the group consisting of C₃₋₆cycloalkyl and four to six-membered oxygen-containing heterocyclyl ring.

In one embodiment, R₃ is a four to six-membered heterocyclyl ring having one to two heteroatom ring members independently selected from O and N, and the heterocyclyl ring is optionally substituted with one to three substituents independently selected from the group consisting of:
halo;
cyano;
C₁₋₃alkyl optionally substituted with one to three substituents independently selected from halo, hydroxyl and C₁₋₃alkoxyl; and
four to six-membered heterocyclyl ring having one to two heteroatom ring members independently selected from O and N.

In a particular embodiment, the four to six-membered heterocyclyl ring having one to two heteroatom ring members independently selected from O and N is attached to the pyrazole ring via a carbon atom.

In one embodiment, R₃ is a four to six-membered heterocyclyl ring having 0 as the heteroatom ring member.

In one embodiment, R₃ is selected from the group consisting of oxetanyl, tetrahydrofuranyl, tetrahydro-2H-pyranyl, wherein the heterocyclyl ring is optionally substituted with one to three substituents independently selected from the group consisting of
hydroxyl, halo;
C₁₋₃alkyl optionally substituted with one to three substituents independently selected from halo, hydroxyl and C₁₋₃alkoxyl;
C₁₋₃alkoxyl optionally substituted with one to three substituents independently selected from halo, hydroxyl and C₁₋₃alkoxyl; and
four to six-membered heterocyclyl ring having one to two heteroatom ring members independently selected from O and N.

In one embodiment, R₃ is a four to six-membered oxygen-containing heterocyclyl ring, which heterocycyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl. In a more particular embodiment, R₃ is selected from the group consisting of oxetanyl, tetrahydrofuranyl and tetrahydro-2H-pyranyl, wherein the oxetanyl, tetrahydrofuranyl or tetrahydro-2H-pyranyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl. Even more particularly, R₃ is selected from the group consisting of oxetan-3-yl, tetrahydrofuran-3-yl, tetrahydro-2H-pyran-3-yl and tetrahydro-2H-pyran-4-yl, wherein the oxetan-3-yl, tetrahydrofuran-3-yl, tetrahydro-2H-pyran-3-yl or tetrahydro-2H-pyran-4-yl ring is optionally substituted with one to three substituents independently selected from the group consisting of halo and C₁₋₃alkyl. Even more particularly, R₃ is selected from the group consisting of oxetan-3-yl, tetrahydrofuran-3-yl, tetrahydro-2H-pyran-3-yl and tetrahydro-2H-pyran-4-yl, wherein the oxetan-3-yl, tetrahydrofuran-3-yl, tetrahydro-2H-pyran-3-yl or tetrahydro-2H-pyran-4-yl ring is optionally substituted with one to three substituents independently selected from the group consisting of fluoro and methyl.

In one embodiment, R₃ is a four to six-membered heterocyclyl ring having one heteroatom ring member independently selected from O and N, wherein the heterocyclyl ring is optionally substituted with a four to six-membered heterocyclyl ring having one heteroatom ring member independently selected from O and N.

In one embodiment, R₃ is a four to six-membered heterocyclyl ring having N as the heteroatom ring member.

In one embodiment, R₃ is a four to six-membered heterocyclyl ring selected from the group consisting of azetidinyl, pyrrolidinyl and piperidinyl, wherein the heterocyclyl ring is optionally substituted with one to three substituents independently selected from the group consisting of:
hydroxyl, halo;
C1-3alkyl optionally substituted with one to three substituents independently selected from halo, hydroxyl and C₁₋₃alkoxyl;
C1-3alkoxyl optionally substituted with one to three substituents independently selected from halo, hydroxyl and C₁₋₃alkoxyl; and
four to six-membered heterocyclyl ring having one to two heteroatom ring members independently selected from O and N.

In one embodiment, R₃ is a four to six-membered nitrogen-containing heterocyclyl ring that is attached to the pyrazole ring by a carbon atom, which nitrogen-containing heterocyclyl ring is optionally substituted with one to three substituents independently selected from the group consisting of:
halo;
C₁₋₃alkyl which alkyl group is optionally substituted with one to three substituents independently selected from halo, hydroxyl and C₁₋₃alkoxyl, and
four to six-membered heterocyclyl ring having one to two heteroatom ring members independently selected from O and N.

In one embodiment, R₃ is a four to six-membered nitrogen-containing heterocyclyl ring, which heterocycyl ring is:
substituted on the nitrogen ring atom by a substituent selected from the group consisting of an C₁₋₃alkyl group which alkyl group is optionally substituted with one to three substituents independently selected from halo, hydroxyl and C₁₋₃alkoxyl, and a four to six-membered heterocyclyl ring having one to two heteroatom ring members independently selected from O and N; and
optionally further substituted with one or two groups independently selected from halo and C₁₋₃alkyl.

In one embodiment, R₃ is a four to six-membered nitrogen-containing heterocyclyl ring, which heterocycyl ring is:
substituted on the nitrogen ring atom by a four to six-membered heterocyclyl ring having one to two heteroatom ring members independently selected from O and N; and
optionally further substituted with one or two groups independently selected from halo and C₁₋₃alkyl.

In one embodiment, R₃ is a four to six-membered nitrogen-containing heterocyclyl ring, which heterocycyl ring is:
substituted on the nitrogen ring atom with one four to six-membered oxygen containing heterocyclyl ring; and
optionally further substituted with one or two groups independently selected from halo and C₁₋₃alkyl.

In one embodiment, R₃ is an azetidinyl or piperidinyl ring, which azetidinyl or piperidinyl ring is:
substituted on the nitrogen ring atom with a tetrahydro-2H-pyranyl, tetrahydrofuranyl or oxetanyl ring; and
optionally further substituted with one or two groups independently selected from halo and C₁₋₃alkyl.

In one embodiment, R₃ is an azetidin-3-yl, piperidin-3-yl or piperidin-4-yl ring, which ring is:
substituted on the nitrogen ring atom with a tetrahydro-2H-pyran-4-yl, tetrahydro-2H-pyran-3-yl, tetrahydrofuran-3-yl or oxetan-3-yl ring; and
optionally further substituted with one or two groups independently selected from halo and C₁₋₃alkyl.

In one embodiment, R₃ is a four to six-membered heterocyclyl ring selected from the group consisting of oxetanyl, azetidinyl, tetrahydrofuranyl, tetrahydro-2H-pyranyl, pyrrolidinyl, piperidinyl and morpholinyl, and the heterocyclyl is optionally substituted with one to three substituents independently selected from halo, methyl, methoxyethyl and oxetanyl.

In one embodiment, R₃ is a four to six-membered heterocyclyl selected from the group consisting of oxetan-3-yl, tetrahydrofuran-2-yl, tetrahydrofuran-3-yl, tetrahydro-2H-pyran-3-yl, tetrahydro-2H-pyran-4-yl, azetidin-3-yl, pyrrolidin-1-yl, pyrrolidin-3-yl, piperidin-3-yl, piperidin-4-yl, morpholin-2-yl, and morpholin-4-yl, wherein the heterocyclyl ring is optionally substituted with one to three substituents independently selected from the group consisting of halo, C₁₋₃alkyl, methoxyethyl and oxetanyl, or pharmaceutically acceptable salts thereof.

In one embodiment, R₃ is C₁₋₆alkyl optionally substituted with one to three substituents independently selected from the group consisting of
CN, hydroxyl, halo, C₁₋₄alkoxyl;
—CO-Q, wherein Q is C₁₋₄alkoxyl, hydroxyl or NR_{c}R_{d}, wherein R_{c} and R_{d} are independently H or C₁₋₄alkyl;

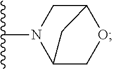 [SMILES: *N1CC2CC1CO2]

and
a 4-7 membered heterocyclyl ring selected from the group consisting of morpholinyl, pyrrolidinyl, tetrahydropyranyl or oxetanyl, which heterocyclyl ring is optionally substituted with one to three halo substituents.

In one embodiment, R₃ is C₁₋₆alkyl optionally substituted with one CN group.

In one embodiment, R₃ is 2-cyano-2-propyl.

In one embodiment, R₃ is C₄₋₆ cycloalkyl optionally substituted with one to three substituents independently selected from the group consisting of
halo, hydroxyl;

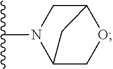 [SMILES: *N1CC2CC1CO2]

C₁₋₃alkyl optionally substituted with one to three substituents independently selected from halo, hydroxyl and C₁₋₃alkoxyl;
C₁₋₄alkoxyl optionally substituted with one to three substituents independently selected from halo, hydroxyl and C₁₋₃alkoxyl; and
four to six-membered heterocyclyl ring having one to two heteroatom ring members independently selected from O and N, wherein the heterocyclyl ring is optionally substituted with one to three halo substituents;

In one embodiment, R₃ is C₄₋₆ cycloalkyl substituted with one hydroxyl group. In a particular embodiment, the hydroxyl group is located at a position that is distal (“para”) to the point of attachment of the cycloalkyl group to the pyrazole ring. In a more particular embodiment, R₃ is 4-hydroxylcyclohexyl.

In one embodiment, R₃ is C₄₋₆ cycloalkyl substituted with one four to six-membered heterocyclyl ring having one to two heteroatom ring members independently selected from O and N, wherein the heterocyclyl ring is optionally substituted with one to three halo substituents. In one embodiment, the heterocyclyl group is linked to the cycloalkyl group by a ring nitrogen.

In one embodiment, R₃ is C₄₋₆ cycloalkyl optionally substituted with one to three substituents independently selected from hydroxyl, morpholinyl or

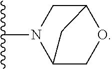 [SMILES: *N1CC2CC1CO2]

In one embodiment, R₃ is C₄₋₆cycloalkyl substituted with one substituent selected from hydroxyl, morpholinyl or

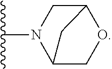 [SMILES: *N1CC2CC1CO2]

In certain embodiments in which the substitutent is morpholinyl, the attachment to the cycloalkyl group is via the nitrogen atom.

In one embodiment, R₃ is cyclohexyl substituted at the 4 position by hydroxyl, morpholin-4-yl or

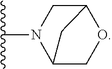 [SMILES: *N1CC2CC1CO2]

In another embodiment, R₃ is cyclobutyl substituted at the 3 position by hydroxyl, morpholin-4-yl or

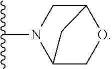 [SMILES: *N1CC2CC1CO2]

In one embodiment, R₃ is a bridged or spiro cyclic ring selected from the group consisting of

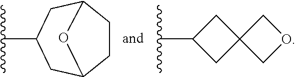 [SMILES: *C1CC2CCC(C1)O2 ; *C1CC2(COC2)C1]

In one embodiment, R₃ is
1) H;
2) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, and the heterocyclyl ring is optionally substituted with one to three substituents independently selected from the group consisting of
   halo;
   oxetanyl; and
   C₁₋₃alkyl optionally substituted with one to three substituents independently selected from halo, hydroxyl and C₁₋₃alkoxyl;
3) 2-cyano-2-propyl;
4) C₄₋₆ cycloalkyl optionally substituted with one to three substituents independently selected from hydroxyl, morpholinyl and

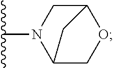 [SMILES: *N1CC2CC1CO2]

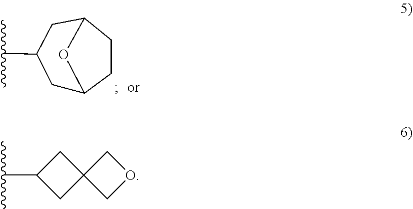 [SMILES: *C1CC2CCC(C1)O2 ; *C1CC2(COC2)C1]

In one embodiment, R₃ is
1) H;
2) four to six-membered oxygen-containing heterocyclyl ring, which heterocyclyl ring is optionally substituted with one to three substituents independently selected from the group consisting of halo, cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is:
   substituted on the nitrogen ring atom by a substituent selected from the group consisting of a C₁₋₃alkyl group which alkyl group is optionally substituted with one to three substituents independently selected from halo, hydroxyl and C₁₋₃alkoxyl, and a four to six-membered heterocyclyl ring having one to two heteroatom ring members independently selected from O and N; and optionally further substituted with one or two groups independently selected from halo and C₁₋₃alkyl.
4) C₁₋₆alkyl optionally substituted with one CN group; or
5) C₄₋₆ cycloalkyl optionally substituted with one to three substituents independently selected from hydroxyl, morpholin-4-yl or

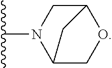 [SMILES: *N1CC2CC1CO2]

In one embodiment, n is 3.

In certain embodiments, R₄ and R₅, at each occurrence, are each independently selected from the group consisting of
H, halo, hydroxyl;
C₁₋₄alkyl optionally substituted with one to three substituents independently selected from halo and C₁₋₄alkoxyl;
C₃₋₆cycloalkyl; and
C₁₋₄alkoxyl optionally substituted with one to three substituents independently selected from halo and C₁₋₄alkoxyl.

In one embodiment, R₄ and R₅, at each occurrence, are each independently selected from the group consisting of H, halo, C₁₋₃alkyl, methoxyl, ethoxyl, fluoromethyl, difluoromethyl, trifluoromethyl, difluoromethoxyl, methoxymethyl and cyclopropyl.

In one embodiment, this invention relates to compounds of Formula (I), and any of the applicable embodiments above, (CR₄R₅)ₙ is CHR₄CHR₅CH₂, CR₄R₅CHR₅CH₂, or CHR₄CR₄R₅CH₂, wherein R₄ and R₅ are defined above.

In certain embodiments, R₄ and R₅, at each occurrence, are each independently selected from the group consisting of H, F, methyl, ethyl, methoxyl, fluoromethyl, difluoromethyl, trifluoromethyl, and methoxymethyl.

In one embodiment, A-(CR₄R₅)ₙ—O is A-CHR₄CHR₅CH₂—O wherein either:

R₄ and R₅ are both H; or

R₄ is H and R₅ is fluoro, C₁₋₄alkyl or C₁₋₄alkoxyl wherein said alkyl or alkoxyl is optionally substituted by one two or three fluoro groups; or

R₄ is cyclopropyl, C₁₋₄alkyl or C₁₋₄alkoxyl wherein said alkyl or alkoxyl is optionally substituted by one two or three fluoro or C₁₋₄ alkoxyl groups and R₅ is H; or

R₄ is C₁₋₄alkyl or C₁₋₄alkoxyl wherein said alkyl or alkoxyl group is optionally substituted by one two or three fluoro groups and R₅ is fluoro; or

R₄ and R₅ are both methyl.

In one embodiment, A-(CR₄R₅)ₙ—O is A-CHR₄CHR₅CH₂—O wherein either:

R₄ and R₅ are both H; or

R₄ is H and R₅ is methyl, methoxyl, ethoxyl, difluoromethoxyl or fluoro; or

R₄ is methyl, ethyl, isopropyl, fluoromethyl, difluromethyl, trifluoromethyl, methoxy, methoxymethyl or cyclopropyl and R₅ is H; or

R₄ is methyl or fluoromethyl and R₅ is fluoro; or

R₄ and R₅ are both methyl.

In one embodiment, A-(CR₄R₅)ₙ—O is A-CHR₄CHR₅CH₂—O wherein either:

R^{a} and R₅ are both H; or

R₄ is H and R₅ is methoxyl or fluoro; or

R₄ is methyl, ethyl, fluoromethyl, difluromethyl, or methoxymethyl and R₅ is H; or

R₄ is methyl and R₅ is fluoro; or

R₄ and R₅ are both methyl.

In certain embodiments, A is NH, X is N, and n is 3.

In certain embodiment, the compound of Formula (I) has the structure of Formula (IA)

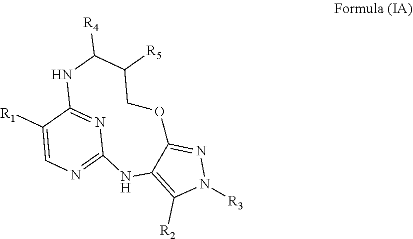

wherein

R₁, R₂, R₃, R₄, R₅ are as defined above in relation to Formula (I).

In one embodiment, the compound of Formula (I) has the structure of Formula (IA) wherein R₁, R₂ and R₃ are as defined above in relation to Formula (I) and wherein either:

R₄ and R₅ are both H; or

R₄ is H and R₅ is fluoro, C₁₋₄alkyl or C₁₋₄alkoxyl wherein said alkyl or alkoxyl is optionally substituted by one two or three fluoro groups; or

R₄ is cyclopropyl, C₁₋₄alkyl or C₁₋₄alkoxyl wherein said alkyl or alkoxyl is optionally substituted by one two or three fluoro or C₁₋₄ alkoxyl groups and R₅ is H; or

R₄ is C₁₋₄alkyl or C₁₋₄alkoxy wherein said alkyl or alkoxy group is optionally substituted by one two or three fluoro groups and R₅ is fluoro; or

R₄ and R₅ are both methyl.

In one embodiment, the compound of Formula (I) has the structure of Formula (IA) wherein R₁, R₂ and R₃ are as defined above in relation to Formula (I) and wherein either:

R₄ and R₅ are both H; or

R₄ is H and R₅ is methyl, methoxyl, ethoxyl, difluoromethoxyl or fluoro; or

R₄ is methyl, ethyl, isopropyl, fluoromethyl, difluromethyl, trifluoromethyl, methoxy, methoxymethyl or cyclopropyl and R₅ is H; or

R₄ is methyl or fluoromethyl and R₅ is fluoro; or

R₄ and R₅ are both methyl.

In one embodiment, the compound of Formula (I) has the structure of Formula (IA) wherein R₁, R₂ and R₃ are as defined above in relation to Formula (I) and wherein either:

R₄ and R₅ are both H; or

R₄ is H and R₅ is methoxyl or fluoro; or

R₄ is methyl, ethyl, fluoromethyl, difluromethyl, or methoxymethyl and R₅ is H; or

R₄ is methyl and R₅ is fluoro; or

R₄ and R₅ are both methyl.

In particular embodiments relating to Formula (IA):

R₁ is selected from the group consisting of H, halo, CN, methyl, isopropyl, tert-butyl, methoxy, trifluoromethyl, trifluoromethoxyl, ethenyl, prop-1-en-2-yl, ethynyl and cyclopropyl;

R₂ is selected from the group consisting of H, halo, CN, methyl, ethyl, difluoromethyl, trifluoromethyl, cyclopropyl, methoxymethyl and methoxyethyl; and

R₃ is
1) H;
2) four to six-membered oxygen-containing heterocyclyl ring, which heterocyclyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is:
   substituted on the nitrogen ring atom by a substitutent selected from the group consisting of a C₁₋₃alkyl group which alkyl group is optionally substituted with one to three substituents independently selected from halo, hydroxyl and C₁₋₃ alkoxyl, and a four to six-membered heterocyclyl ring having one to two heteroatom ring members independently selected from O and N; and optionally further substituted with one or two groups independently selected from halo and C₁₋₃alkyl.
4) C₁₋₆alkyl optionally substituted with one CN group; or
5) C₄₋₆ cycloalkyl optionally substituted with one to three substituents independently selected from hydroxyl, morpholin-4-yl or

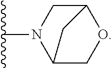 [SMILES: *N1CC2CC1CO2]

In more particular embodiments relating to Formula (IA):

R₁ is selected from the group consisting of Br, Cl and CN;

R₂ is selected from the group consisting of H Cl, CN and methyl; and

R₃ is
1) H;
2) four to six-membered oxygen-containing heterocyclyl ring, which heterocyclyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is:
   substituted on the nitrogen ring atom by a substituent selected from the group consisting of an C₁₋₃alkyl group which alkyl group is optionally substituted with one to three substituents independently selected from halo, hydroxyl and C₁₋₃alkoxyl, and a four to six-membered heterocyclyl ring having one to two heteroatom ring members independently selected from O and N; and
   optionally further substituted with one or two groups independently selected from halo and C₁₋₃alkyl.
4) C₁₋₆alkyl optionally substituted with one CN group; or
5) C₄₋₆ cycloalkyl optionally substituted with one to three substituents independently selected from hydroxyl, morpholin-4-yl or

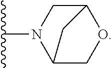 [SMILES: *N1CC2CC1CO2]

In an even more particular embodiment relating to Formula (IA):

R₁ is selected from the group consisting of Br, Cl and CN;

R₂ is selected from the group consisting of H Cl, CN and methyl; and

R₃ is
1) H;
2) four to six-membered oxygen-containing heterocyclyl ring, which heterocyclyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is substituted on the nitrogen ring atom with one four to six-membered oxygen-containing heterocyclyl ring, and optionally further substituted with one or two groups independently selected from halo and C₁₋₃alkyl; or
4) C₄₋₆ cycloalkyl optionally substituted with one to three substituents independently selected from hydroxyl, morpholin-4-yl or

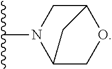 [SMILES: *N1CC2CC1CO2]

In one embodiment, the compound of Formula (I) has the structure of Formula (IA) wherein:
R₁ is Br, Cl or CN;
R₂ is Cl, CN or methyl;
R₃ is
1) H
2) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, wherein the heterocyclyl ring is optionally substituted with one to three substituents independently selected from halo, methyl and oxetanyl;
3) 2-cyano-2-propyl;
4) C₄₋₆ cycloalkyl optionally substituted with one to three substituents independently selected from hydroxyl, morpholinyl and

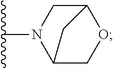 [SMILES: *N1CC2CC1CO2]

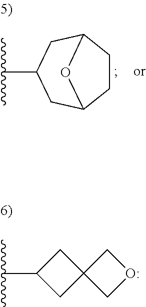

R₄ is H, methyl, ethyl, fluoromethyl, difluoromethyl, trifluoromethyl, or methoxymethyl; and
R₅ is H, F, methyl, or methoxyl.

In one embodiment, this invention relates to compounds of Formula (I) or Formula (IA), and any of the applicable embodiments above, wherein R₁ is Br, Cl or CN, R₂ is Cl, CN or methyl, and R₃ is
1) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, wherein the heterocyclyl ring is optionally substituted with one to three substituents independently selected from halo, methyl, methoxyethyl and oxetanyl;
2) 2-cyano-2-propyl;
3) C₄₋₆ cycloalkyl optionally substituted with one to three substituents independently selected from hydroxyl, morpholinyl and

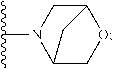 [SMILES: *N1CC2CC1CO2]

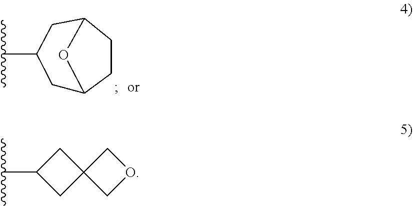 [SMILES: *C1CC2CCC(C1)O2 ; *C1CC2(COC2)C1]

R₄ is H, methyl, ethyl, fluoromethyl, difluoromethyl, trifluoromethyl, or methoxymethyl; and R₅ is H, F, methyl, or methoxyl.

In one embodiment, the invention relates to a compound Formula (IA)

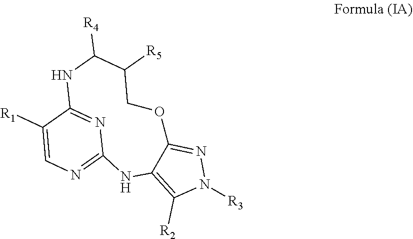

wherein

R₁ is selected from the group consisting of Br, Cl and CN;

R₂ is selected from the group consisting of H Cl, CN and methyl;

R₃ is
1) H;
2) four to six-membered oxygen-containing heterocyclyl ring, which heterocyclyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is substituted on the nitrogen ring atom with one four to six-membered oxygen containing heterocyclyl ring, and optionally further substituted with one or two groups independently selected from halo and C₁₋₃alkyl; or
4) C₄₋₆ cycloalkyl optionally substituted with one to three substituents independently selected from hydroxyl, morpholin-4-yl or

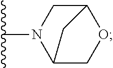 [SMILES: *N1CC2CC1CO2]

and either

R₄ and R₅ are both H; or

R₄ is H and R₅ is methoxyl or fluoro; or

R₄ is methyl, ethyl, fluoromethyl, difluromethyl, or methoxymethyl and R₅ is H; or

R₄ is methyl and R₅ is fluoro; or

R₄ and R₅ are both methyl;

or a pharmaceutically acceptable salt thereof.

In a certain embodiment, this invention relates to a compound selected from

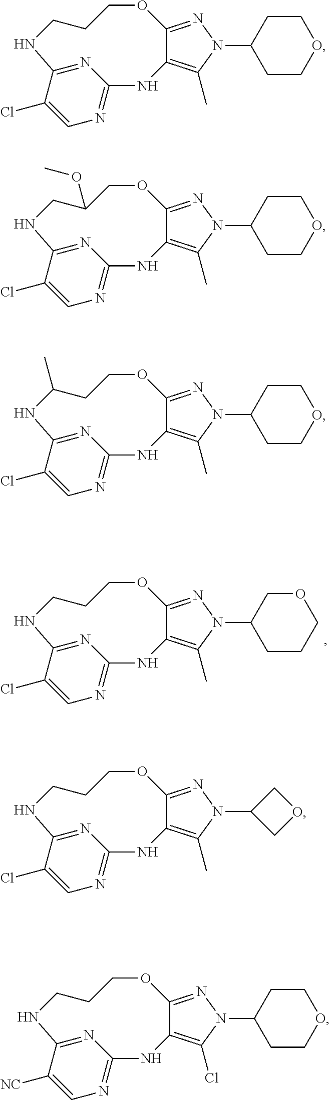 [SMILES: Cc1c2c(nn1C1CCOCC1)OCCCNc1nc(ncc1Cl)N2 ; COC1CNc2nc(ncc2Cl)Nc2c(nn(C3CCOCC3)c2C)OC1 ; Cc1c2c(nn1C1CCOCC1)OCCC(C)Nc1nc(ncc1Cl)N2 ; Cc1c2c(nn1C1CCCOC1)OCCCNc1nc(ncc1Cl)N2 ; Cc1c2c(nn1C1COC1)OCCCNc1nc(ncc1Cl)N2 ; N#Cc1cnc2nc1NCCCOc1nn(C3CCOCC3)c(Cl)c1N2]

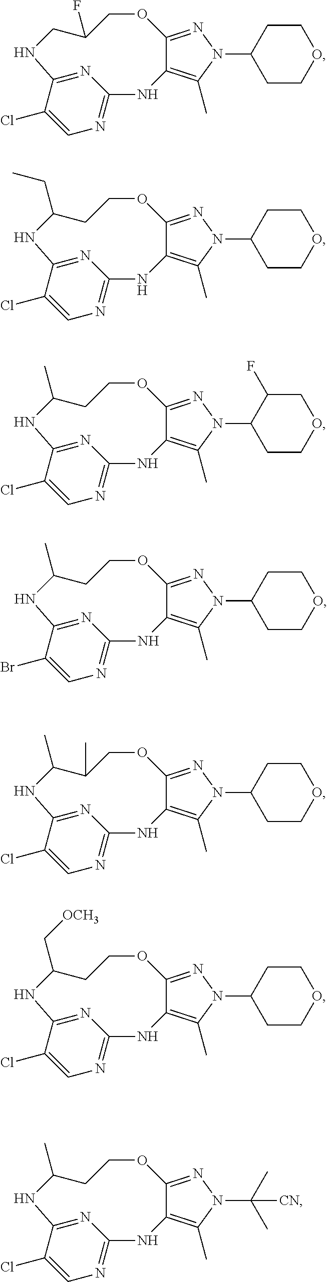

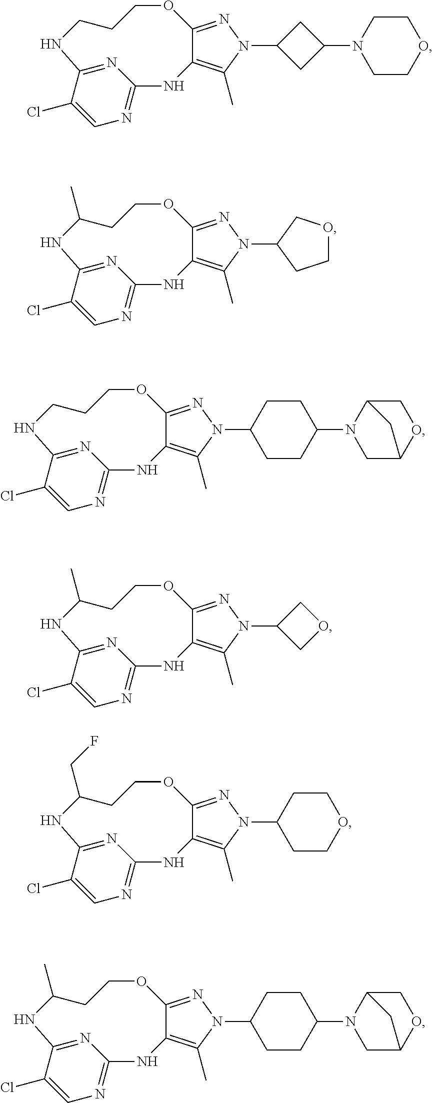

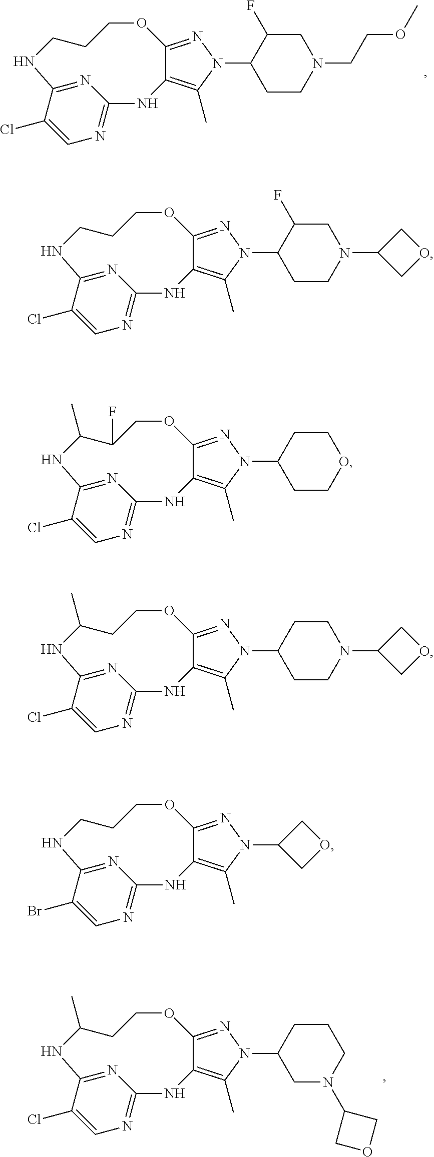 [SMILES: COCCN1CCC(n2nc3c(c2C)Nc2ncc(Cl)c(n2)NCCCO3)C(F)C1 ; Cc1c2c(nn1C1CCN(C3COC3)CC1F)OCCCNc1nc(ncc1Cl)N2 ; Cc1c2c(nn1C1CCOCC1)OCC(F)C(C)Nc1nc(ncc1Cl)N2 ; Cc1c2c(nn1C1CCN(C3COC3)CC1)OCCC(C)Nc1nc(ncc1Cl)N2 ; Cc1c2c(nn1C1COC1)OCCCNc1nc(ncc1Br)N2 ; Cc1c2c(nn1C1CCCN(C3COC3)C1)OCCC(C)Nc1nc(ncc1Cl)N2]

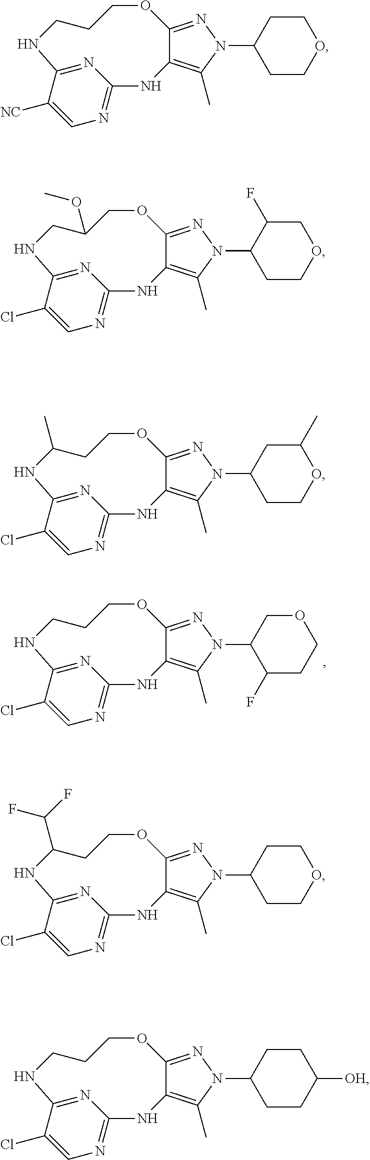 [SMILES: Cc1c2c(nn1C1CCOCC1)OCCCNc1nc(ncc1C#N)N2 ; COC1CNc2nc(ncc2Cl)Nc2c(nn(C3CCOCC3F)c2C)OC1 ; Cc1c2c(nn1C1CCOC(C)C1)OCCC(C)Nc1nc(ncc1Cl)N2 ; Cc1c2c(nn1C1COCCC1F)OCCCNc1nc(ncc1Cl)N2 ; Cc1c2c(nn1C1CCOCC1)OCCC(C(F)F)Nc1nc(ncc1Cl)N2 ; Cc1c2c(nn1C1CCC(O)CC1)OCCCNc1nc(ncc1Cl)N2]

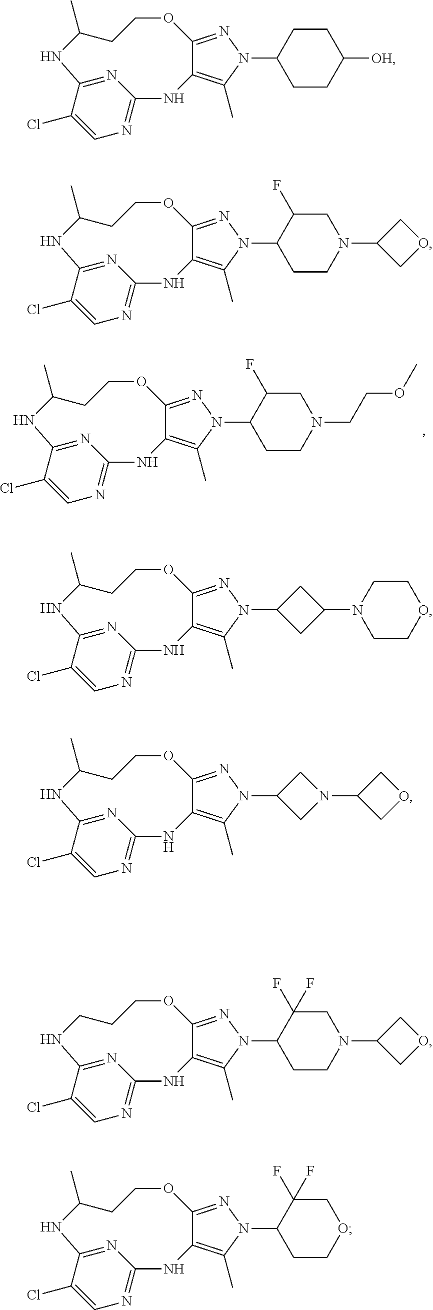

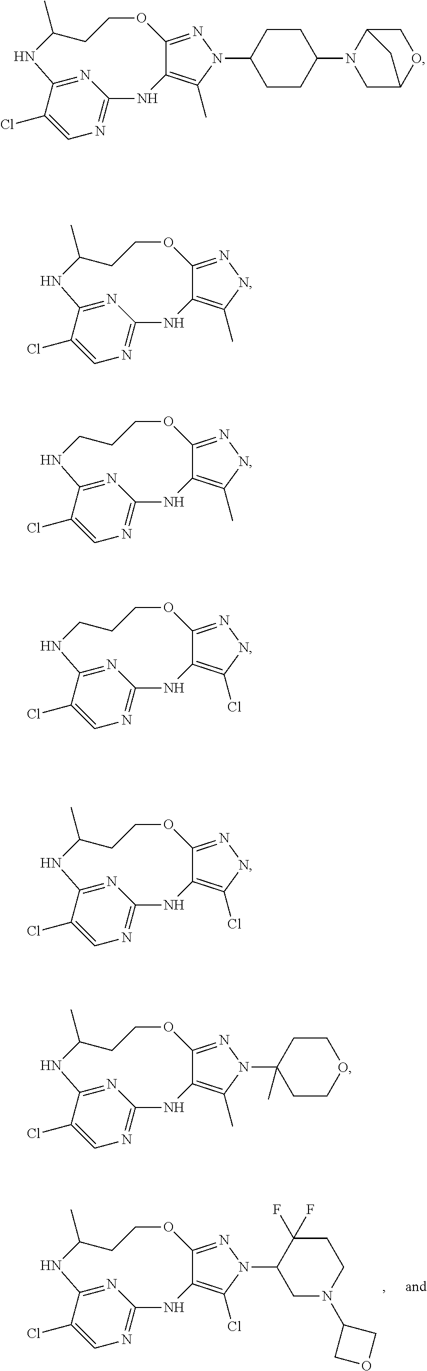

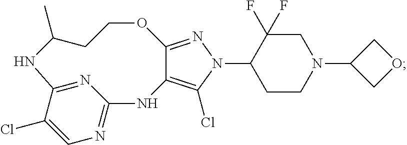 [SMILES: CC1CCOc2nn(C3CCN(C4COC4)CC3(F)F)c(Cl)c2Nc2ncc(Cl)c(n2)N1]

or a pharmaceutically acceptable salt thereof.

In another embodiment, this invention relates to a compound selected from

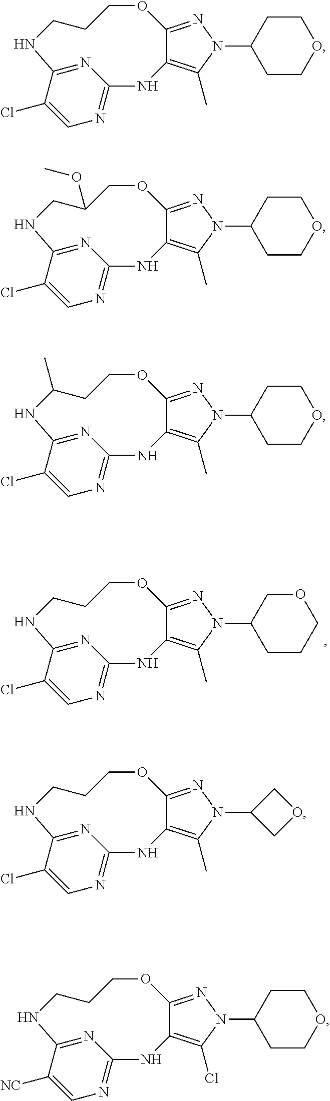 [SMILES: Cc1c2c(nn1C1CCOCC1)OCCCNc1nc(ncc1Cl)N2 ; COC1CNc2nc(ncc2Cl)Nc2c(nn(C3CCOCC3)c2C)OC1 ; Cc1c2c(nn1C1CCOCC1)OCCC(C)Nc1nc(ncc1Cl)N2 ; Cc1c2c(nn1C1CCCOC1)OCCCNc1nc(ncc1Cl)N2 ; Cc1c2c(nn1C1COC1)OCCCNc1nc(ncc1Cl)N2 ; N#Cc1cnc2nc1NCCCOc1nn(C3CCOCC3)c(Cl)c1N2]

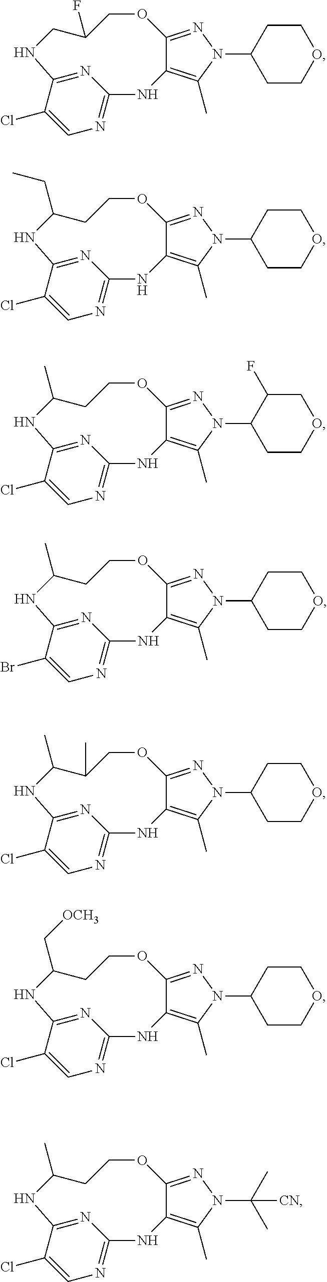

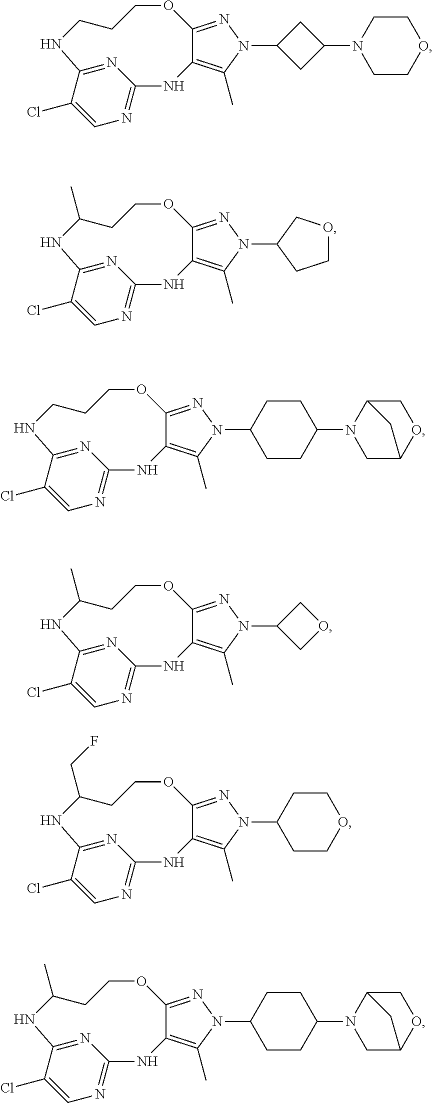

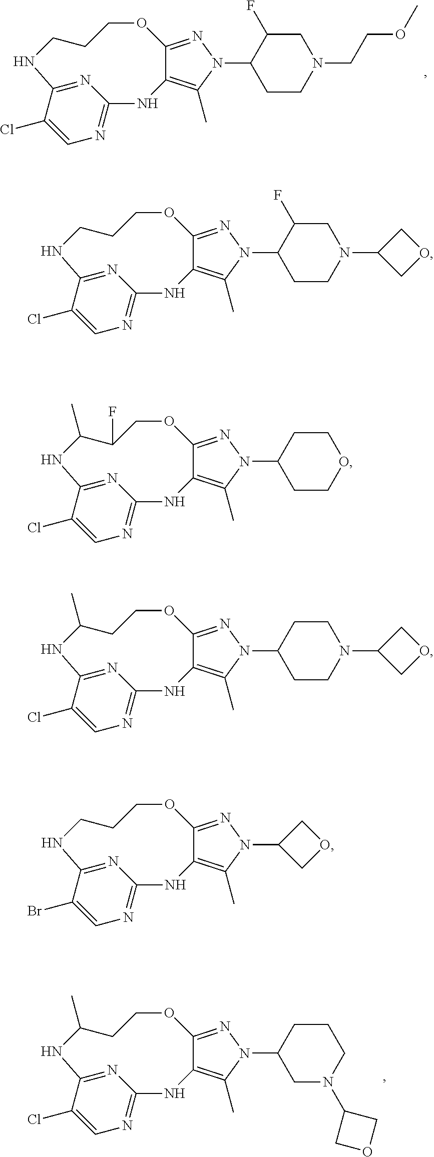 [SMILES: COCCN1CCC(n2nc3c(c2C)Nc2ncc(Cl)c(n2)NCCCO3)C(F)C1 ; Cc1c2c(nn1C1CCN(C3COC3)CC1F)OCCCNc1nc(ncc1Cl)N2 ; Cc1c2c(nn1C1CCOCC1)OCC(F)C(C)Nc1nc(ncc1Cl)N2 ; Cc1c2c(nn1C1CCN(C3COC3)CC1)OCCC(C)Nc1nc(ncc1Cl)N2 ; Cc1c2c(nn1C1COC1)OCCCNc1nc(ncc1Br)N2 ; Cc1c2c(nn1C1CCCN(C3COC3)C1)OCCC(C)Nc1nc(ncc1Cl)N2]

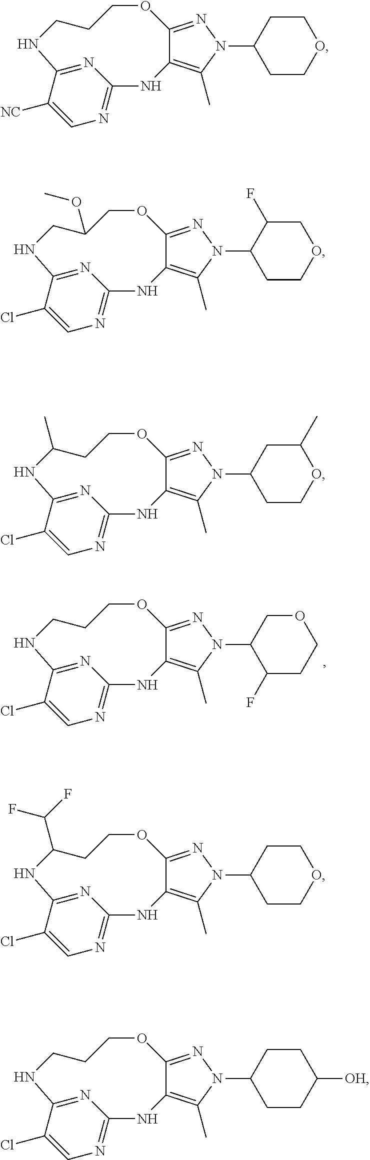 [SMILES: Cc1c2c(nn1C1CCOCC1)OCCCNc1nc(ncc1C#N)N2 ; COC1CNc2nc(ncc2Cl)Nc2c(nn(C3CCOCC3F)c2C)OC1 ; Cc1c2c(nn1C1CCOC(C)C1)OCCC(C)Nc1nc(ncc1Cl)N2 ; Cc1c2c(nn1C1COCCC1F)OCCCNc1nc(ncc1Cl)N2 ; Cc1c2c(nn1C1CCOCC1)OCCC(C(F)F)Nc1nc(ncc1Cl)N2 ; Cc1c2c(nn1C1CCC(O)CC1)OCCCNc1nc(ncc1Cl)N2]

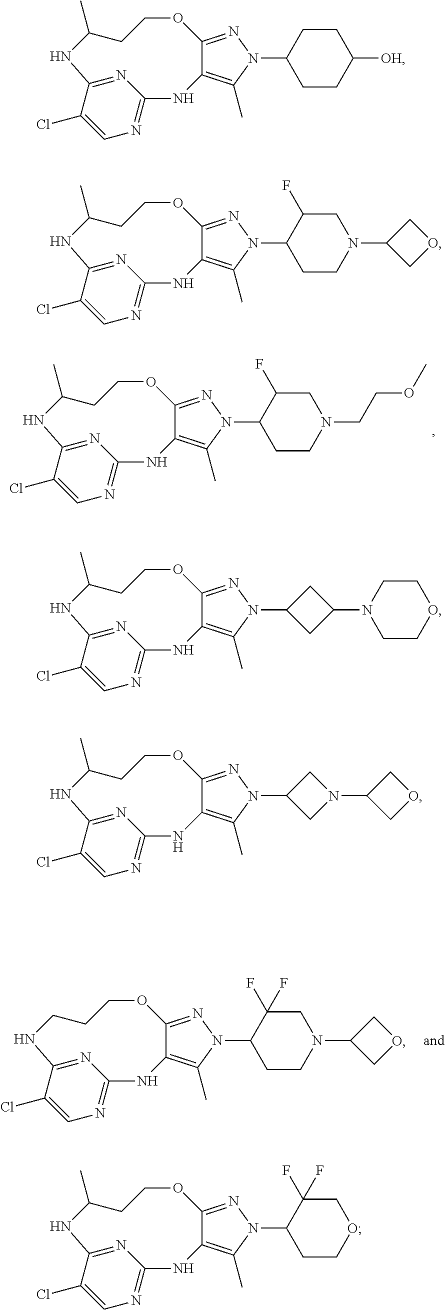

or a pharmaceutically acceptable salt thereof.

In one embodiment, this invention relates to a compound selected from

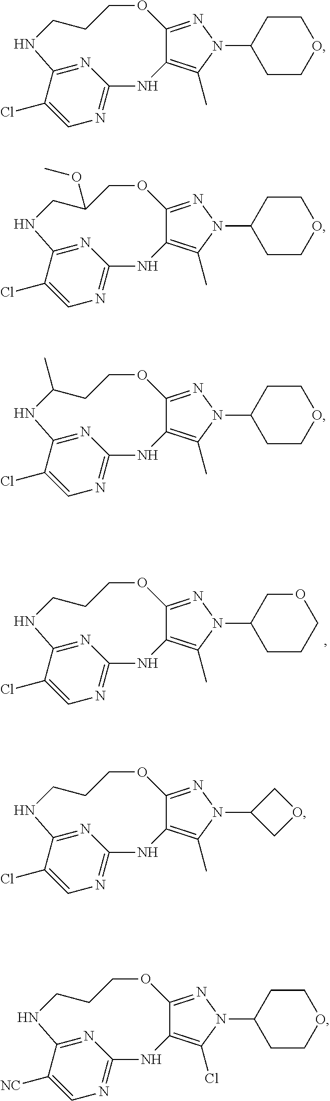 [SMILES: Cc1c2c(nn1C1CCOCC1)OCCCNc1nc(ncc1Cl)N2 ; COC1CNc2nc(ncc2Cl)Nc2c(nn(C3CCOCC3)c2C)OC1 ; Cc1c2c(nn1C1CCOCC1)OCCC(C)Nc1nc(ncc1Cl)N2 ; Cc1c2c(nn1C1CCCOC1)OCCCNc1nc(ncc1Cl)N2 ; Cc1c2c(nn1C1COC1)OCCCNc1nc(ncc1Cl)N2 ; N#Cc1cnc2nc1NCCCOc1nn(C3CCOCC3)c(Cl)c1N2]

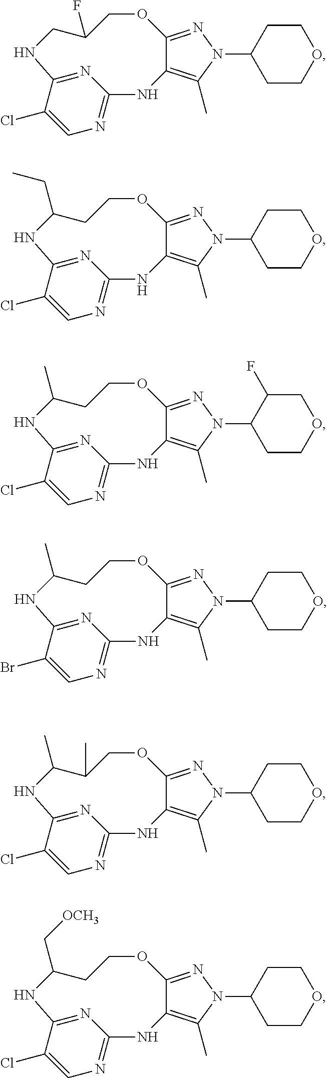

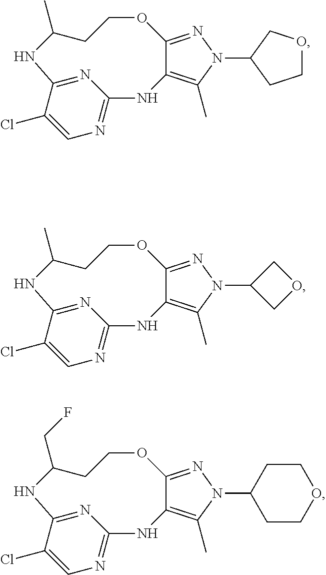

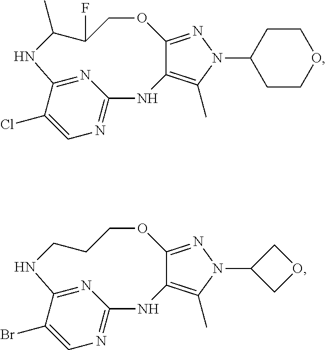 [SMILES: Cc1c2c(nn1C1CCOCC1)OCC(F)C(C)Nc1nc(ncc1Cl)N2 ; Cc1c2c(nn1C1COC1)OCCCNc1nc(ncc1Br)N2]

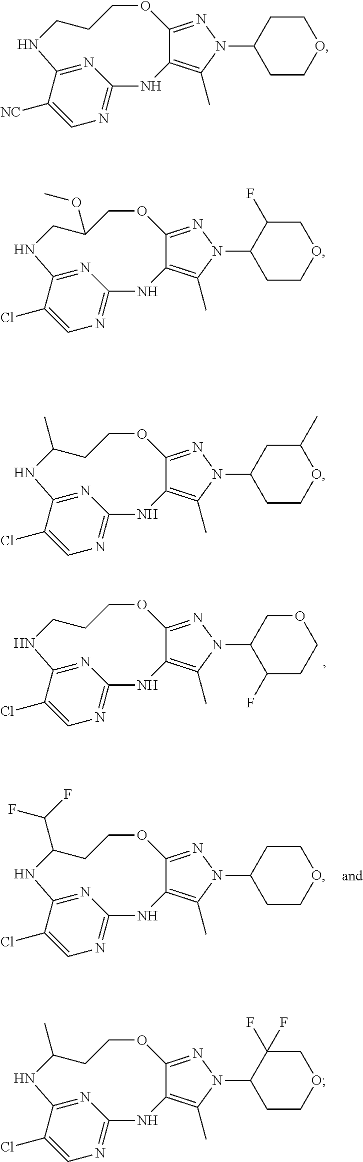 [SMILES: Cc1c2c(nn1C1CCOCC1)OCCCNc1nc(ncc1C#N)N2 ; COC1CNc2nc(ncc2Cl)Nc2c(nn(C3CCOCC3F)c2C)OC1 ; Cc1c2c(nn1C1CCOC(C)C1)OCCC(C)Nc1nc(ncc1Cl)N2 ; Cc1c2c(nn1C1COCCC1F)OCCCNc1nc(ncc1Cl)N2 ; Cc1c2c(nn1C1CCOCC1)OCCC(C(F)F)Nc1nc(ncc1Cl)N2 ; Cc1c2c(nn1C1CCOCC1(F)F)OCCC(C)Nc1nc(ncc1Cl)N2]

or a pharmaceutically acceptable salt thereof.

In one embodiment, this invention relates to a compound selected from

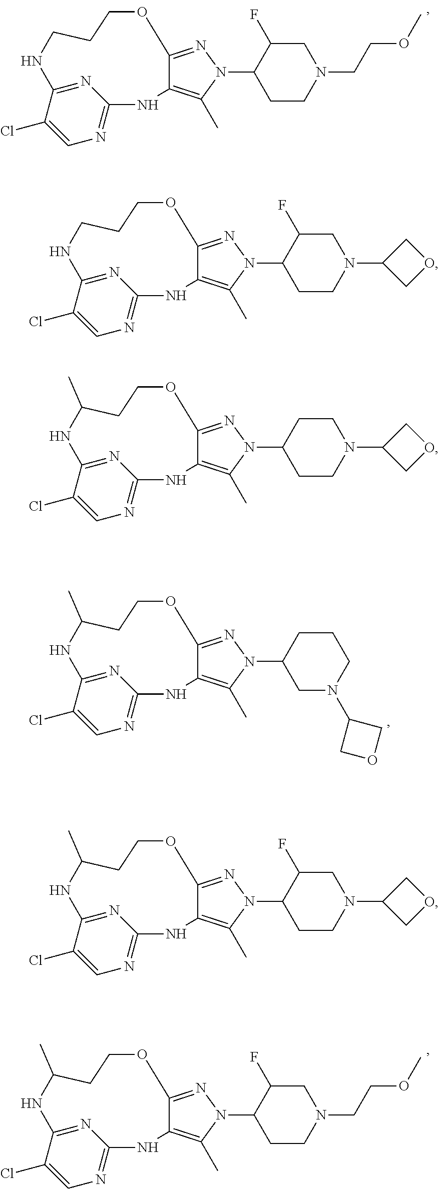 [SMILES: COCCN1CCC(n2nc3c(c2C)Nc2ncc(Cl)c(n2)NCCCO3)C(F)C1 ; Cc1c2c(nn1C1CCN(C3COC3)CC1F)OCCCNc1nc(ncc1Cl)N2 ; Cc1c2c(nn1C1CCN(C3COC3)CC1)OCCC(C)Nc1nc(ncc1Cl)N2 ; Cc1c2c(nn1C1CCCN(C3COC3)C1)OCCC(C)Nc1nc(ncc1Cl)N2 ; Cc1c2c(nn1C1CCN(C3COC3)CC1F)OCCC(C)Nc1nc(ncc1Cl)N2 ; COCCN1CCC(n2nc3c(c2C)Nc2ncc(Cl)c(n2)NC(C)CCO3)C(F)C1]

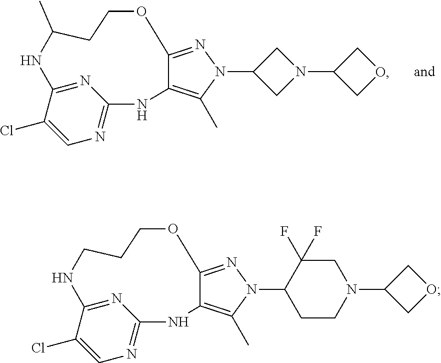 [SMILES: Cc1c2c(nn1C1CN(C3COC3)C1)OCCC(C)Nc1nc(ncc1Cl)N2 ; Cc1c2c(nn1C1CCN(C3COC3)CC1(F)F)OCCCNc1nc(ncc1Cl)N2]

or a pharmaceutically acceptable salt thereof.

In one embodiment, this invention relates to a compound selected from

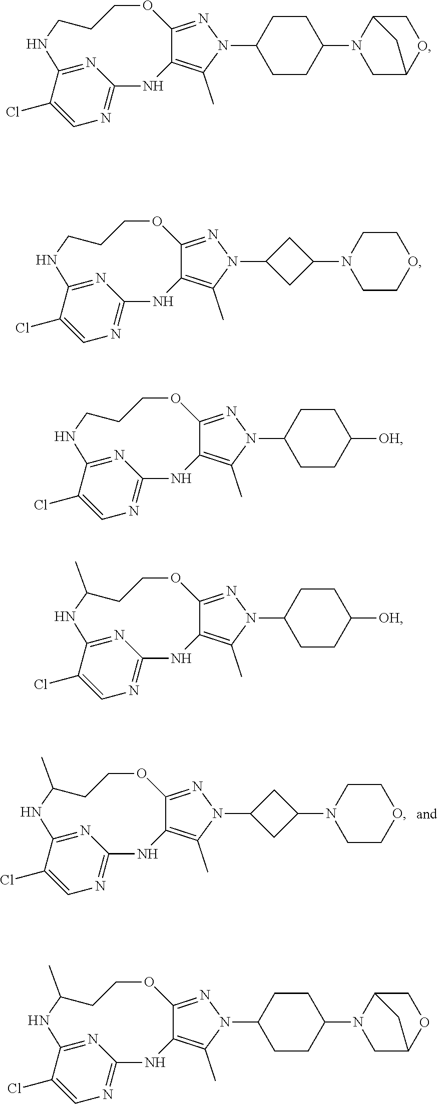

or a pharmaceutically acceptable salt thereof.

In one embodiment, this invention relates to

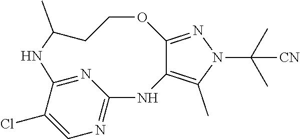 [SMILES: Cc1c2c(nn1C(C)(C)C#N)OCCC(C)Nc1nc(ncc1Cl)N2]

or a pharmaceutically acceptable salt thereof.

In one embodiment, this invention relates to

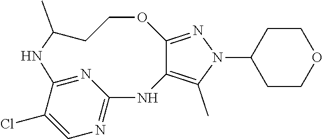 [SMILES: Cc1c2c(nn1C1CCOCC1)OCCC(C)Nc1nc(ncc1Cl)N2]

or a pharmaceutically acceptable salt thereof.

In one embodiment, this invention relates to

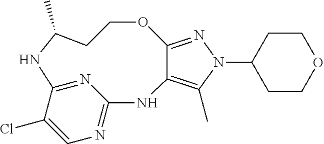 [SMILES: Cc1c2c(nn1C1CCOCC1)OCC[C@@H](C)Nc1nc(ncc1Cl)N2]

or a pharmaceutically acceptable salt thereof.

In one embodiment, the invention relates to a crystalline form of (11R)-14-chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene which exhibits characteristic XRPD peaks at 8.7, 10.1, 10.5, 13.9, 15.9, 17.4, 18.2, 18.7, 19.6, 25.3 and 27.0 (±0.2°) when measured using a copper anode that generates Kα radiation with a wavelength of 1.5406 Å. In a particular embodiment, the peaks are identified in a PANalytical X'Pert Pro powder diffractometer, model PW3040/60 using an X'Celerator detector with radiation: Cu Kα, generator tension: 40 kV, generator current: 45 mA, start angle: 2.0° 2θ, end angle: 40.0° 2θ, step size: 0.0167° 2θ, time per step: 31.75 seconds.

In a certain embodiment, the invention relates to compounds of Formula (I) wherein R₃ is H.

In a certain embodiment, this invention relates to a compound selected from

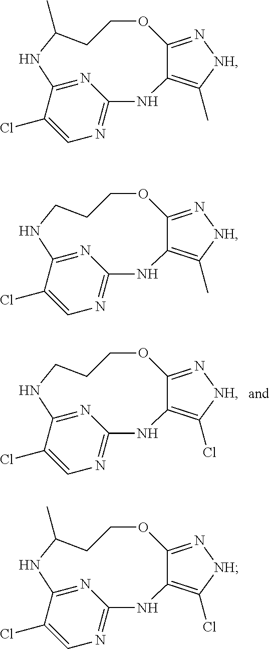

or pharmaceutically acceptable salt thereof.

In one embodiment, this invention relates to:

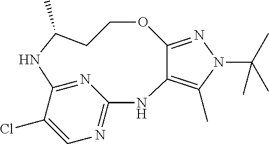 [SMILES: Cc1c2c(nn1C(C)(C)C)OCC[C@@H](C)Nc1nc(ncc1Cl)N2]

or a pharmaceutically acceptable salt thereof.

In one embodiment, the invention provides a compound of Formula (I) or a salt thereof which is the compound of any one of Examples E1-E452, or a salt thereof.

It is to be understood that the references herein to a compound of Formula (I) or a salt thereof includes a compound of Formula (I) as a free base or acid, or as a salt thereof, for example as a pharmaceutically acceptable salt thereof. Thus, in one embodiment, the invention is directed to a compound of Formula (I). In another embodiment, the invention is directed to a salt of a compound of Formula (I). In a further embodiment, the invention is directed to a pharmaceutically acceptable salt of a compound of Formula (I). In another embodiment, the invention is directed to a compound of Formula (I) or a salt thereof. In a further embodiment, the invention is directed to a compound of Formula (I) or a pharmaceutically acceptable salt thereof.

Because of its potential use in medicine, it will be appreciated that a salt of a compound of Formula (I) is preferably pharmaceutically acceptable.

As used herein, the term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Pharmaceutically acceptable salts are within the scope of the present invention. Pharmaceutically acceptable salts include, amongst others, those described in Berge et al, J. Pharm, Sci., 66, 1-19, 1977, those in P L Gould, International Journal of Pharmaceutics, 33 (1986), 201-217; Bighley et al, Encyclopedia of Pharmaceutical Technology, Marcel Dekker Inc, New York 1996, Volume 13, page 453-497, or those in P H Stahl and C G Wermuth, editors, Handbook of Pharmaceutical Salts; Properties, Selection and Use, Second Edition Stahl/Wermuth: Wiley—VCH/VHCA, 2011 (see http://www.wiley.com/VVileyCDA/WileyTitle/productCd-3906390519.html).

Non-pharmaceutically acceptable salts are within the scope of the present invention, for example for use as intermediates in the preparation of a compound of Formula (I) or a pharmaceutically acceptable salt thereof.

Non-pharmaceutically acceptable salts may be used, for example as intermediates in the preparation of a compound of Formula (I) or a pharmaceutically acceptable salt thereof.

The compounds of Formula (I) contain a basic group and are therefore capable of forming pharmaceutically-acceptable acid addition salts by treatment with a suitable acid. Suitable acids include pharmaceutically-acceptable inorganic acids and pharmaceutically-acceptable organic acids. Exemplary pharmaceutically-acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate. In certain embodiments, some of these salts may form solvates. In certain embodiments, some of these salts may be crystalline.

Such acid addition salts can be formed by reaction of a compound of Formula (I) (which, for example contains a basic amine or other basic functional group) with the appropriate acid, optionally in a suitable solvent such as an organic solvent, to give the salt which can be isolated by a variety of methods, including crystallisation and filtration.

It will be understood that if a compound of Formula (I) contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of formula (I) are included within the scope of the invention, including sub-stoichiometric salts, for example where a counterion contains more than one acidic proton.

Certain compounds of Formula (I) contain an acidic group and are therefore capable of forming pharmaceutically-acceptable base addition salts by treatment with a suitable base. Suitable bases include pharmaceutically-acceptable inorganic bases and pharmaceutically-acceptable organic bases. Exemplary pharmaceutically-acceptable acid addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

Such base addition salts can be formed by reaction of a compound of Formula (I) (which, for example, contains an acidic functional group) with the appropriate base, optionally in a suitable solvent such as an organic solvent, to give the salt which can be isolated by a variety of methods, including crystallisation and filtration.

Salts may be prepared in situ during the final isolation and purification of a compound of Formula (I). If a basic compound of Formula (I) is isolated as a salt, the corresponding free base form of that compound may be prepared by any suitable method known to the art, including treatment of the salt with an inorganic or organic base. Similarly, if a compound of Formula (I) containing an acidic functional group is isolated as a salt, the corresponding free acid form of that compound may be prepared by any suitable method known to the art, including treatment of the salt with an inorganic or organic acid.

Certain compounds of Formula (I) or salts thereof may exist in stereoisomeric forms (e.g., they may contain one or more asymmetric carbon atoms). The individual stereoisomers (enantiomers and diastereomers) and mixtures of these are included within the scope of the present invention. The different isomeric forms may be separated or resolved one from the other by conventional methods, or any given isomer may be obtained by conventional synthetic methods or by stereospecific or asymmetric syntheses.

Certain compounds of Formula (I) are capable of existing in tautomeric forms. For example, certain compounds exhibit amine-imine tautomerism (such as

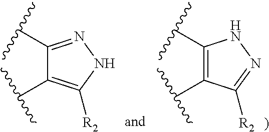

and may exist in one or more of the tautomeric forms. In some cases, only one of a pair of tautomeric forms (for example,

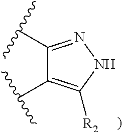

falls within Formula (I) as drawn. The other alternative tautomer (for example,

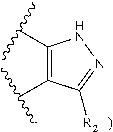

also forms part of the invention.

The invention also includes isotopically-labelled compounds and salts, which are identical to compounds of Formula (I) or salts thereof, but for the fact that one or more atoms are replaced by an atom having an atomic mass or mass number different from the atomic mass or mass number most commonly found in nature. Examples of isotopes that can be incorporated into compounds of Formula (I) or salts thereof isotopes of hydrogen, carbon, nitrogen, fluorine, such as ³H, ¹¹C, ¹⁴C and ¹⁸F. Such isotopically-labelled compound of Formula (I) or salts thereof are useful in drug and/or substrate tissue distribution assays. For example, ¹¹C and ¹⁸F isotopes are useful in PET (positron emission tomography). PET is useful in brain imaging. Isotopically-labelled compounds of Formula (I) and salts thereof can generally be prepared by carrying out the procedures disclosed below, by substituting a readily available isotopically-labelled reagent for a non-isotopically labelled reagent. In one embodiment, compounds of Formula (I) or salts thereof are not isotopically labelled.

Certain compounds of Formula (I) or salts thereof may exist in solid or liquid form. In the solid state, certain compounds of Formula (I) or salts thereof may exist in crystalline or noncrystalline form, or as a mixture thereof. For compounds of Formula (I) or salts thereof that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

The skilled artisan will further appreciate that any compounds of Formula (I) or pharmaceutically acceptable salts thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The skilled artisan also appreciates that this invention may contain various deuterated forms of compounds of Formula (I), or pharmaceutically acceptable salts thereof. Each available hydrogen atom attached to a carbon atom may be independently replaced with a deuterium atom. A person of ordinary skill in the art will know how to synthesize deuterated forms of compounds of Formula (I), or pharmaceutically acceptable salts thereof. Commercially available deuterated starting materials may be employed in the preparation of deuterated forms of compounds of Formula (I) or pharmaceutically acceptable salts thereof, or they may be synthesized using conventional techniques employing deuterated reagents (e.g. lithium aluminum deuteride).

### C. Methods of Use

Compounds of Formula (I) or pharmaceutically acceptable salts thereof are inhibitors of LRRK2 kinase activity and are thus believed to be of potential use in the treatment of or prevention of the following neurological diseases associated with or characterized by LRRK2 kinase activity: Parkinson's disease, Alzheimer's disease, amyotrophic lateral sclerosis (ALS), dementia (including Lewy body dementia and vascular dementia, HIV-induced dementia), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis. Other diseases potentially treatable by inhibition of LRRK2 include, but are not limited to, lysosomal disorders (for example, Niemann-Pick Type C disease, Gaucher disease), Crohn's disease, inflammatory bowel disease (IBD), cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, bacterial infections (including leprosy), viral infections (including tuberculosis, HIV, West Nile virus and chikungunya virus) and parasitic infections.

One aspect of the invention provides a compound of Formula (I) or a pharmaceutically acceptable salt thereof for use in therapy. In one embodiment, the invention provides a compound of Formula (I) or a pharmaceutically acceptable salt thereof for use in the treatment of or prevention of the above disorders (i.e. the neurological diseases and other diseases listed above). In one embodiment, the invention provides a compound of Formula (I) or a pharmaceutically acceptable salt thereof for use in the treatment of or prevention of Parkinson's disease. In one embodiment, the invention provides a compound of Formula (I) or a pharmaceutically acceptable salt thereof for use in the treatment of Parkinson's disease. In another embodiment, the invention provides a compound of Formula (I) or a pharmaceutically acceptable salt thereof for use in the treatment of or prevention of Alzheimer's disease. In one embodiment, the invention provides a compound of Formula (I) or a pharmaceutically acceptable salt thereof for use in the treatment of Alzheimer's disease. In another embodiment, the invention provides a compound of Formula (I) or a pharmaceutically acceptable salt thereof for use in the treatment of tuberculosis.

A further aspect of the invention provides the use of a compound of Formula (I) or a pharmaceutically acceptable salt thereof in the manufacture of a medicament for the treatment of or prevention of of the above disorders (i.e. the neurological diseases and other diseases listed above). A further aspect of the invention provides the use of a compound of Formula (I) or a pharmaceutically acceptable salt thereof in the manufacture of a medicament for the treatment of or prevention of Parkinson's disease. A further aspect of the invention provides the use of a compound of Formula (I) or a pharmaceutically acceptable salt thereof in the manufacture of a medicament for the treatment of Parkinson's disease. In another embodiment, the invention provides the use of a compound of Formula (I) or a pharmaceutically acceptable salt thereof in the manufacture of a medicament for the treatment of treatment of or prevention of Alzheimer's disease. In one embodiment, the invention provides the use of a compound of Formula (I) or a pharmaceutically acceptable salt thereof in the manufacture of a medicament for the treatment of Alzheimer's disease. In another embodiment, the invention provides use of a compound of Formula (I) or a pharmaceutically acceptable salt thereof in the manufacture of a medicament for the treatment of tuberculosis.

A further aspect of the invention provides a method of treatment or prevention of a disorder listed above (i.e. selected from the neurological diseases and other diseases listed above), which comprises administering to a subject in need thereof a therapeutically effective amount of a compound of Formula (I) or a pharmaceutically acceptable salt thereof. A further aspect of the invention provides a method of treatment or prevention of Parkinson's disease, which comprises administering to a subject in need thereof a therapeutically effective amount of a compound of Formula (I) or a pharmaceutically acceptable salt thereof. A further aspect of the invention provides a method of treatment of Parkinson's disease, which comprises administering to a subject in need thereof a therapeutically effective amount of a compound of Formula (I) or a pharmaceutically acceptable salt thereof. In an embodiment, the subject is human.

In the context of the present invention, treatment of Parkinson's disease refers to the treatment of sporadic Parkinson's disease, and/or familial Parkinson's disease. In one embodiment, treatment of Parkinson's disease refers to treatment of familial Parkinson's disease.

In one embodiment, familial Parkinson's disease patients are those expressing at least one of the following LRRK2 kinase mutations: G2019S mutation, N1437H mutation, R1441G mutation, R1441C mutation, R1441H mutation, Y1699C mutation, S1761R mutation, or I2020T mutation. In another embodiment, familial Parkinson's disease patients express other coding mutations (such as G2385R) or non-coding single nucleotide polymorphisms at the LRRK2 locus that are associated with Parkinson's disease. In a more particular embodiment, familial Parkinson's disease includes patients expressing at least the G2019S mutation or the R1441G mutation in LRRK2 kinase. In one embodiment, treatment of Parkinson's disease refers to the treatment of familial Parkinson's disease which includes patients expressing LRRK2 kinase bearing at least the G2019S mutation. In another embodiment, familial Parkinson's disease patients express aberrantly high levels of wild-type LRRK2 kinase.

In another embodiment, familial Parkinson's disease patients are those expressing one or more of the following LRRK2 kinase mutations: G2019S mutation, N1437H mutation, R1441G mutation, R1441C mutation, R1441H mutation, Y1699C mutation, S1761R mutation, or I2020T mutation. In another embodiment, familial Parkinson's disease patients express other coding mutations (such as G2385R) or non-coding single nucleotide polymorphisms at the LRRK2 locus that are associated with Parkinson's disease. In a more particular embodiment, familial Parkinson's disease includes patients expressing the G2019S mutation or the R1441G mutation in LRRK2 kinase. In one embodiment, treatment of Parkinson's disease refers to the treatment of familial Parkinson's disease includes patients expressing LRRK2 kinase bearing G2019S mutation. In another embodiment, familial Parkinson's disease patients express aberrantly high levels of normal LRRK2 kinase.

Treatment of Parkinson's disease may be symptomatic or may be disease modifying. In one embodiment, treatment of Parkinson's disease refers to symptomatic treatment. In one embodiment, treatment of Parkinson's disease refers to disease modifying treatment. In one embodiment, treatment of Parkinson's disease refers to both symptomatic treatment and disease modifying treatment.

Compounds of the present invention may also be useful in treating patients identified as susceptible to progression to severe Parkinsonism by means of one or more subtle features associated with disease progression, such as family history, olfaction deficits, constipation, cognitive defects, gait or biological indicators of disease progression gained from molecular, biochemical, immunological or imaging technologies. In this context, treatment may be symptomatic or disease modifying. In another embodiment, treatment may be symptomatic and disease modifying.

A further aspect of the invention provides a method of treatment or prevention of Alzheimer's disease, which comprises administering to a subject in need thereof a therapeutically effective amount of a compound of Formula (I) or a pharmaceutically acceptable salt thereof.

A further aspect of the invention provides a method of treatment of Alzheimer's disease, which comprises administering to a subject in need thereof a therapeutically effective amount of a compound of Formula (I) or a pharmaceutically acceptable salt thereof. A further aspect of the invention provides a method of treatment of tuberculosis, which comprises administering to a subject in need thereof a therapeutically effective amount of a compound of Formula (I) or a pharmaceutically acceptable salt thereof. In an embodiment, the subject is human.

In the context of the present invention, treatment of Alzheimer's disease refers to the treatment of sporadic Alzheimer's disease and/or familial Alzheimer's disease. Treatment of Alzheimer's disease may be symptomatic and/or disease modifying. In one embodiment, treatment of Alzheimer's disease may be symptomatic or may be disease modifying. In one embodiment, treatment of Alzheimer's disease refers to symptomatic treatment.

A further aspect of the invention provides a method of treatment or prevention of amyotrophic lateral sclerosis (ALS) disease, which comprises administering to a subject in need thereof a therapeutically effective amount of a compound of Formula (I) or a pharmaceutically acceptable salt thereof. A further aspect of the invention provides a method of treatment of amyotrophic lateral sclerosis (ALS) disease, which comprises administering to a subject in need thereof a therapeutically effective amount of a compound of Formula (I) or a pharmaceutically acceptable salt thereof. In an embodiment, the subject is human.

In the context of the present invention, treatment of amyotrophic lateral sclerosis (ALS), dementia (including Lewy body dementia and vascular dementia, HIV-induced dementia), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), multiple sclerosis, lysosomal disorders (for example, Niemann-Pick Type C disease, Gaucher disease), Crohn's disease, inflammatory bowel disease (IBD), cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjögren's syndrome, Devic's disease, inflammatory myopathies, ankylosing spondylitis, may be symptomatic and/or disease modifying. In one embodiment, treatment of these disorders may be symptomatic or disease modifying. In certain embodiments, treatment of these disorders refers to symptomatic treatment.

The invention also provides the use of inhibitors of LRRK2 in the production of neuronal progenitor cells in vitro for consequent therapeutic application in cell based-treatment of CNS disorders.

When a compound of Formula (I) or a pharmaceutically acceptable salt thereof is intended for use in the treatment of Parkinson's disease, it may be used in combination with medicaments alleged to be useful as symptomatic treatments of Parkinson's disease. Suitable examples of such other therapeutic agents include L-dopa, and dopamine agonists (e.g. pramipexole, ropinirole). Accordingly, in one embodiment, the invention provides a combination of a compound of formula (I) or a pharmaceutically acceptable salt thereof with an active pharmaceutical ingredient selected from the list consisting of L-dopa and a dopamine agonist (e.g. pramipexole, ropinirole).

When a compound of Formula (I) or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride rivastigmine, and galantamine), nicotinic receptor agonists or allosteric modulators (such as 07 agonists or allosteric modulators or α4β2 agonists or allosteric modulators), PPAR agonists (such as PPARγ agonists), 5-HT4 receptor partial agonists, 5-HT6 receptor antagonists e.g. SB-742457 or 5HT1A receptor antagonists and NMDA receptor antagonists or modulators, or disease modifying agents such as β or γ-secretase inhibitors e.g semagacestat, mitochondrial stabilizers, microtubule stabilizers or modulators of Tau pathology such as Tau aggregation inhibitors (e.g. methylene blue and REMBER™), NSAIDS, e.g. tarenflurbil, tramiprosil; or antibodies for example bapineuzumab or solanezumab; proteoglycans, for example tramiprosate. Accordingly, in one embodiment, the invention provides a combination of a compound of formula (I) or a pharmaceutically acceptable salt thereof with an active pharmaceutical ingredient selected from the list consisting of: an M1 muscarinic receptor agonist, an M2 muscarinic antagonist, an acetylcholinesterase inhibitor (e.g. tetrahydroaminoacridine, donepezil hydrochloride rivastigmine, and galantamine), a nicotinic receptor agonist (e.g. an α7 agonist or an α4β2 agonist), a PPAR agonist (e.g. a PPARγ agonist), a 5-HT4 receptor partial agonist, a 5-HT₆ receptor antagonist (e.g. SB-742457), a 5HT1A receptor antagonist, an NMDA receptor antagonist, a β or γ-secretase inhibitor (e.g semagacestat), a mitochondrial stabilizer, a microtubule stabilizer or a Tau aggregation inhibitor (e.g. methylene blue and REMBER™), an NSAID, (e.g. tarenflurbil, tramiprosil), an antibody (for example bapineuzumab or solanezumab) and a proteoglycan (e.g. tramiprosate).

When a compound of Formula (I) or a pharmaceutically acceptable salt thereof is intended for use in the treatment of bacterial infections, parasitic infections or viral infections, it may be used in combination with medicaments alleged to be useful as symptomatic treatments that directly target the infectious agent.

When a compound of Formula (I) or a pharmaceutically acceptable salt thereof is used in combination with other therapeutic agents, the compound may be administered either sequentially or simultaneously by any convenient route.

The invention also provides, in a further aspect, a combination comprising a compound of Formula (I) or a pharmaceutically acceptable salt thereof together with one or more further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of Formula (I) or a pharmaceutically acceptable salt thereof is used in combination with a second therapeutic agent active against the same disease state the dose of each compound may differ from that when the compound is used alone. Appropriate doses will be readily appreciated by those skilled in the art.

### D. Composition

Compounds of Formula (I) or pharmaceutically acceptable salts thereof may be formulated into pharmaceutical compositions prior to administration to a subject. According to one aspect, the invention provides a pharmaceutical composition comprising a compound of Formula (I) or a pharmaceutically acceptable salt thereof and a pharmaceutically acceptable excipient. According to another aspect, the invention provides a process for the preparation of a pharmaceutical composition comprising admixing a compound of Formula (I) or a pharmaceutically acceptable salt thereof, with a pharmaceutically acceptable excipient.

Pharmaceutical compositions may be presented in unit dose forms containing a predetermined amount of active ingredient per unit dose. Such a unit may contain, for example, 0.1 mg, 0.5 mg, or 1 mg to 50 mg, 100 mg, 150 mg, 200 mg, 250 mg, 500 mg, 750 mg or 1 g of a compound of the present invention, depending on the disease being treated, the route of administration and the age, weight and condition of the subject, or pharmaceutical compositions may be presented in unit dose forms containing a predetermined amount of active ingredient per unit dose. In other embodiments, the unit dosage compositions are those containing a daily dose or sub-dose as described herein, or an appropriate fraction thereof, of an active ingredient. Furthermore, such pharmaceutical compositions may be prepared by any of the methods well-known to one skilled in the art.

A therapeutically effective amount of a compound of Formula (I) will depend upon a number of factors including, for example, the age and weight of the intended recipient, the precise condition requiring treatment and its severity, the nature of the formulation, and the route of administration, and will ultimately be at the discretion of the attendant prescribing the medication. However, a therapeutically effective amount of a compound of Formula (I) for the treatment of diseases described in the present invention will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or in a number of sub-doses per day as such as two, three, four, five or six doses per day. Or the dosing can be done intermittently, such as once every other day, once a week or once a month. A therapeutically effective amount of a pharmaceutically acceptable salt or solvate, etc., may be determined as a proportion of the therapeutically effective amount of the compound of Formula (I) per se. It is envisaged that similar dosages would be appropriate for treatment of the other diseases referred to above.

The pharmaceutical compositions of the invention may contain one or more compounds of Formula (I). In some embodiments, the pharmaceutical compositions may contain more than one compound of the invention. For example, in some embodiments, the pharmaceutical compositions may contain two or more compounds of Formula (I). In addition, the pharmaceutical compositions may optionally further comprise one or more additional pharmaceutically active compounds.

In certain embodiments, the pharmaceutical compositions may contain a compound of Formula (I) or Formula (IA), or a pharmaceutically acceptable salt thereof, and a pharmaceutically acceptable excipient.

In certain embodiments, the present invention is directed to a pharmaceutical composition comprising 0.01 to 1000 mg of one or more of a compound of Formula (I) or a pharmaceutically acceptable salt thereof and 0.01 to 5 g of one or more pharmaceutically acceptable excipients.

As used herein, “pharmaceutically acceptable excipient” means a pharmaceutically acceptable material, composition or vehicle involved in giving form or consistency to the pharmaceutical composition. Each excipient may be compatible with the other ingredients of the pharmaceutical composition when commingled such that interactions which would substantially reduce the efficacy of the compound of the invention when administered to a subject and interactions which would result in pharmaceutical compositions that are not pharmaceutically acceptable are avoided.

The compounds of the invention and the pharmaceutically-acceptable excipient or excipients may be formulated into a dosage form adapted for administration to the subject by the desired route of administration. For example, dosage forms include those adapted for (1) oral administration (including buccal or sublingual) such as tablets, capsules, caplets, pills, troches, powders, syrups, elixers, suspensions, solutions, emulsions, sachets, and cachets; (2) parenteral administration (including subcutaneous, intramuscular, intravenous or intradermal) such as sterile solutions, suspensions, and powders for reconstitution; (3) transdermal administration such as transdermal patches; (4) rectal administration such as suppositories; (5) nasal inhalation such as dry powders, aerosols, suspensions, and solutions; and (6) topical administration (including buccal, sublingual or transdermal) such as creams, ointments, lotions, solutions, pastes, sprays, foams, and gels. Such compositions may be prepared by any methods known in the art of pharmacy, for example by bringing into association a compound of Formula (I) with the carrier(s) or excipient(s). Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Suitable pharmaceutically-acceptable excipients may vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically acceptable excipients may be chosen for their ability to facilitate carrying or transporting the compound or compounds of the invention once administered to the subject from an organ, or a portion of the body, to another organ, or a portion of the body. Certain pharmaceutically-acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a therapeutically effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another embodiment, the present invention is directed to a pharmaceutical composition for the treatment of neurodegeneration disease comprising a compound described herein or a pharmaceutically acceptable salt thereof and a pharmaceutically acceptable excipient. In another embodiment, the present invention is directed a pharmaceutical composition for the treatment of Parkinson's disease, Alzheimer's disease or amyotrophic lateral sclerosis (ALS), comprising a compound described herein or a pharmaceutically acceptable salt thereof and a pharmaceutically acceptable excipient.

### E. Process of Preparing Compounds

The process to be utilized in the preparation of compounds of Formula (I) described herein depends upon the desired compounds. Such factors as the selection of the specific substituent and various possible locations of the specific substituent all play a role in the path to be followed in the preparation of the specific compounds of this invention. Those factors are readily recognized by one of ordinary skill in the art.

In general, the compounds of the present invention may be prepared by standard techniques known in the art and by known processes analogous thereto. General methods for preparing compounds of Formula (I) are set forth below. All starting material and reagents described in the below general experimental schemes are commercially available or can be prepared by methods known to one skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

General Schemes 1 and 2 provide exemplary processes of synthesis for preparing compounds of the present invention.

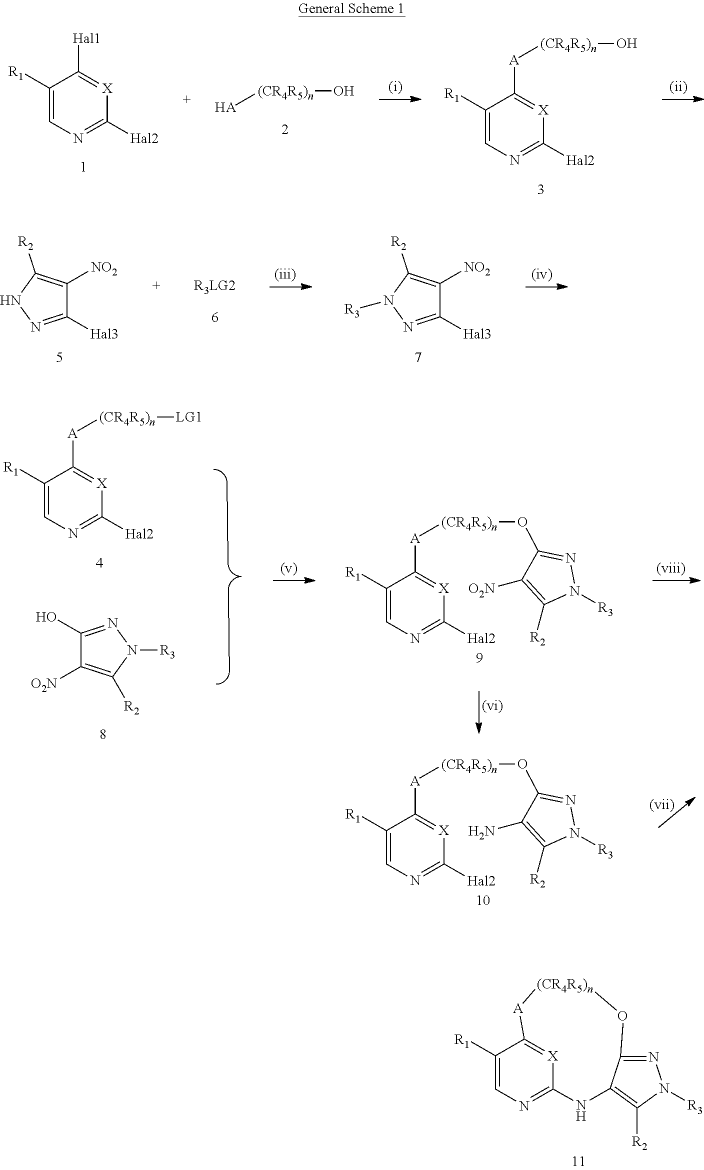

General Scheme 1 provides an exemplary process of synthesis for preparing compound 11, which represents compounds of Formula (I). In Scheme 1, R¹, R², R³, R⁴, R⁵, A, X and n are defined in Formula I. Hal1, Hal2 and Hal3 are halo groups such as Cl, Br, I. LG1 and LG2 are leaving groups such as OMs (methanesulfonate) or OTs (4-methylbenzenesulfonate).

Step (i) may be a substitution reaction by reacting compound 1 with compound 2 in basic conditions (for example, in the presence of a suitable base such as trimethylamine) in a suitable solvent (such as tetrahydrofuran or Tween 20/water solution) at a suitable temperature such as 0° C. to 100° C. to provide intermediate 3.

Intermediate 4 may be obtained in step (ii) by reacting intermediate 3 with a suitable reagent such MsCl (methanesulfonyl chloride) in the presence of a suitable base such as triethylamine in a suitable solvent such as dichloromethane under a suitable temperature such as −20° C. to 40° C.

Step (iii) may be a substitution reaction by reacting compound 5 with compound 6 using a suitable base such as K₂CO₃ in a suitable solvent such as N, N-dimethylformamide at a suitable temperature such as 0° C. to 100° C. to provide intermediate 7.

Intermediate 8 may be obtained in step (iv) by a hydrolysis reaction of intermediate 7 in the presence of a suitable base such as KOH in a suitable solvent such as water under a suitable temperature such as 20° C. to 100° C.

Intermediate 9 may be obtained in step (v) by reacting intermediate 4 with intermediate 8 using a suitable base such as K₂CO₃ in a suitable solvent such as DMF(N, N-dimethylformamide) at a suitable temperature such as 20° C. to 100° C.

Step (vi) may be a reduction reaction of intermediate 9 in the presence of a suitable metal such as Fe and a suitable reagent such as NH₄Cl in a suitable solvent such as EtOH under a suitable temperature such as room temperature to 100° C. to provide intermediate 10.

Step (vii) may be an intro-molecular coupling reaction of intermediate 10 using a suitable catalyst such as Pd₂(dba)₃ in the presence of a suitable base such as K₃PO₄ and a suitable ligand such as 2-dicyclohexylphosphino-2′,4′,6′-triisopropylbiphenyl in a suitable solvent such as dioxane under a suitable temperature such as 90° C. to 120° C. to provide compound 11.

Compound 11 may also be obtained in step (viii) directly by reductive cyclization of intermediate 9 in the presence of a suitable metal such as Fe and a suitable reagent such as NH₄Cl in a suitable solvent (such as EtOH or EtOH/water) under a suitable temperature such as room temperature to 100° C.

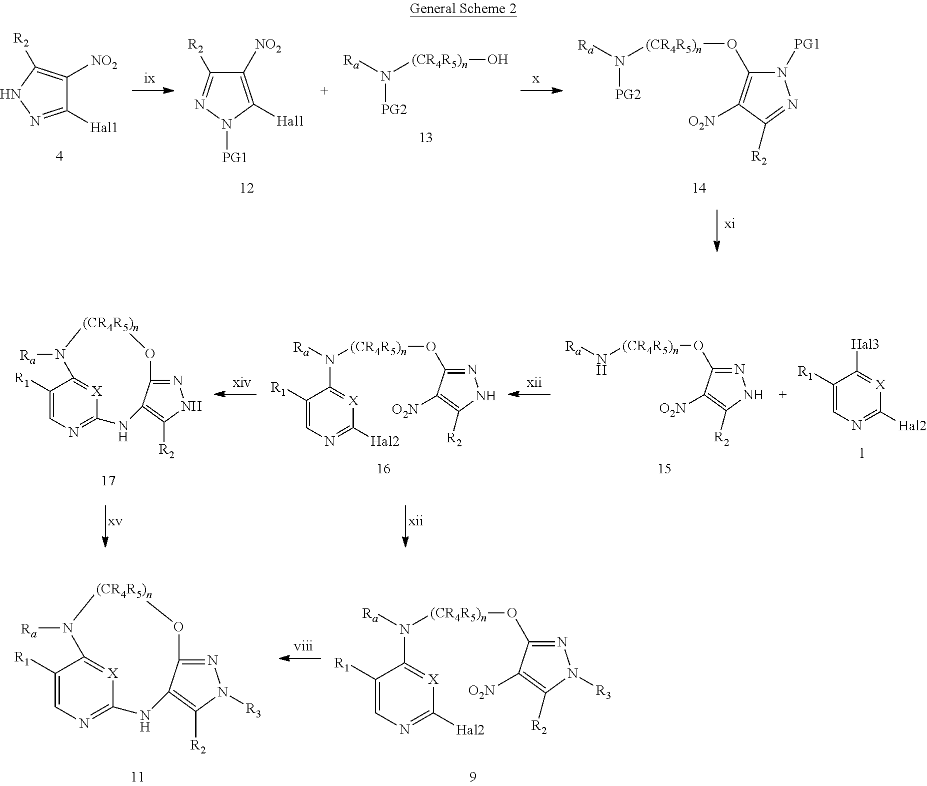

General Scheme 2 provides an exemplary process of synthesis for preparing compound 11, which represents compounds of Formula (I). In Scheme 2, R₁, R₂, R₃, R₄, R⁵, R^{a}, X and n are defined in Formula I. Hal1, Hal2 and Hal3 are halo groups such as Cl, Br, I, PG1 and PG2 are suitable protecting groups such as THP (tetrahydro-2H-pyran), Boc(tert-butyloxycarbonyl) or SEM((trimethylsilyl)ethoxy)methyl).

Step (ix) is a protection reaction, which involves by reacting compound 4 with a suitable reagent such as DHP in the presence of a suitable acid such as TsOH in a suitable solvent such as dichloromethane at a suitable temperature such as 20° C. to 60° C. to provide intermediate 12.

Intermediate 14 may be obtained in step (x) by reacting intermediate 12 with compound 13 in the presence of a suitable base such as NaH in a suitable solvent such as DMF(N, N-dimethylformamide) at a suitable temperature such as 20° C. to 100° C. Step (x) may alternatively be a Ulmann reaction by reacting intermediate 12 with compound 13 in the presence of a suitable catalyst such as CuI, a suitable base such as K₂CO₃ and a suitable ligand in a suitable solvent such as toluene under a suitable temperature such as 90° C. to 120° C. to provide intermediate 14.

Step (xi) is a de-protection reaction, which involves treating intermediate 14 with a suitable acid such HCl in a suitable solvent such as 1,4-dioxane at a suitable temperature such as 25° C. to 40° C. to give intermediate 15.

Step (xii) may be a substitution reaction by reacting compound 1 with intermediate 15 using a suitable base such as DIPEA in a suitable solvent such as i-PrOH at a suitable temperature such as 20° C. to 100° C. to provide intermediate 16.

Intermediate 9 may be obtained in step (xiii) by reacting intermediate 16 with compound 6 using a suitable base such as Cs₂CO₃ in a suitable solvent such as DMF at a suitable temperature such as 20° C. to 100° C., followed by reductive cyclization to provide compound 11 in step (viii).

Step (xiv) may be a reductive cyclization reaction of intermediate 16 in the presence of a suitable metal such as Fe and a suitable reagent such as NH₄Cl in a suitable solvent such as EtOHunder a suitable temperature such as 20° C. to 100° C. to provide compound 17 (which is compound 11 when R₃=H).

Compound 11 may also be obtained in step (xv) by reacting intermediate 17 with a suitable acylating reagent such as cyclopropanecarbonyl chloride in the presence of a suitable base such as Et₃N in a suitable solvent such as dichloromethane under a suitable temperature such as 0° C. to 40° C.

The starting material and reagents described in the above schemes are either commercially available or may be readily prepared from commercially available compounds using procedures known to a person of ordinary skill in the art.

In one embodiment, the invention may be described by the following numbered paragraphs:

Paragraph 1. A compound of Formula (I)

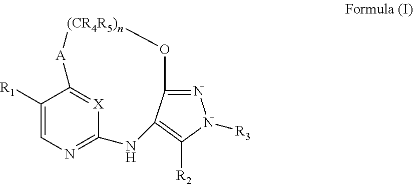

or a pharmaceutically acceptable salt thereof, wherein
X is CH or N;
n is 2, 3, 4 or 5;
A is O or NR^{a}, wherein
   R^{a} is
      H;
      C₁₋₄alkyl optionally substituted with one to three substituents independently selected from the group consisting of halo, hydroxyl and C₁₋₃alkoxyl, wherein C₁₋₃alkoxyl is optionally substituted with one to three halo substituents;
      C₃₋₆cycloalkyl optionally substituted with one to three substituents independently selected from the group consisting of halo, hydroxyl and C₁₋₃alkoxyl; or
      four to seven-membered heterocyclyl ring having one or two heteroatom ring members independently selected from O and N, and the heterocyclyl ring is optionally substituted with one to three substituents independently selected from halo and C₁₋₃alkyl, which C₁₋₃alky is optionally substituted with one to three halo substituents;
R₁ is
   1) H, halo, CN;
   2) C₁₋₄alkyl optionally substituted with one to three substituents independently selected from the group consisting of halo, hydroxyl and C₁₋₃alkoxyl, which C₁₋₃alkoxyl is optionally substituted with one to three halo substituents;
   3) C₂₋₆ alkenyl optionally substituted with one to three halo or C₁₋₃alkyl, which C₁₋₃alkyl is optionally substituted with one to three halo substituents;
   4) C₂₋₆ alkynyl optionally substituted with one to three C₁₋₃alkyl substituents, which C₁₋₃alkyl is optionally substituted with one to three halo substituents;
   5) C₁₋₄alkoxyl optionally substituted with one to three halo substituents;
   6) C₃₋₆cycloalkyl optionally substituted with one to three substituents independently selected from the group consisting of halo, hydroxyl, C₁₋₃alkoxyl and C₁₋₃alkyl, wherein C₁₋₃alkoxyl and C₁₋₃alkyl are optionally substituted with one to three halo substituents;
   7) OC₃₋₆cycloalkyl optionally substituted with one to three substituents independently selected from the group consisting of halo, hydroxyl, C₁₋₃alkoxyl and C₁₋₃alkyl;
   8) four to seven-membered heterocyclyl ring having one or two heteroatom ring members independently selected from O and N, and the heterocyclyl ring is optionally substituted with one to three substituents independently selected from the group consisting of halo, hydroxyl, C₁₋₃alkoxyl and C₁₋₃alkyl;
   9) O-heterocyclyl ring having one or two heteroatom ring members independently selected from O and N, wherein the heterocyclyl ring is a four to seven-membered ring optionally substituted with one to three substituents independently selected from the group consisting of halo, hydroxyl, C₁₋₃alkoxyl and C₁₋₃alkyl; or
   10) SC₁₋₄alkyl optionally substituted with one to three halo substituents;
R₂ is
   H, halo, CN;
   C₁₋₄alkoxyl optionally substituted with one to three halo substituents;
   C₁₋₄alkyl optionally substituted with one to three substituents independently selected from the group consisting of halo, hydroxyl and C₁₋₃alkoxyl, which C₁₋₃alkoxyl is optionally substituted with one to three halo substituents; or
   C₃₋₆cycloalkyl optionally substituted with one to three substituents independently selected from the group consisting of halo, hydroxyl and C₁₋₃alkoxyl;
R₃ is
   1) H;
   2) —CO—Z, wherein Z is selected from the group consisting of
      four to seven-membered heterocyclyl ring having one or two heteroatom ring members independently selected from O and N;
      C₃₋₆cycloalkyl optionally substituted with one to three substituents independently selected from the group consisting of halo, hydroxyl, C₁₋₃alkyl, and C₁₋₃alkoxyl; and
      C₁₋₆alkyl optionally substituted with one to three substituents independently selected from halo and C₁₋₃alkoxyl;
   3) four to seven-membered heterocyclyl ring having one or two heteroatom ring members independently selected from O and N, and the heterocyclyl ring is optionally substituted with one to three substituents independently selected from the group consisting of
      halo;
      C₁₋₃alkyl optionally substituted with one to three substituents independently selected from halo, hydroxyl and C₁₋₃alkoxyl;
      C₁₋₃alkoxyl optionally substituted with one to three substituents independently selected from halo, hydroxyl and C₁₋₃alkoxyl; and
      four to seven-membered heterocyclyl ring having one or two heteroatom ring members independently selected from O and N, and the heterocyclyl ring is optionally substituted with one to three substituents independently selected from the group consisting of halo, C₁₋₃alkyl and C₁₋₃alkoxyl;
   4) C₁₋₆alkyl optionally substituted with one to three substituents independently selected from the group consisting of
      CN, hydroxyl, halo;
      C₁₋₄alkoxyl optionally substituted with one to three substituents independently selected from halo, hydroxyl and C₁₋₃alkoxyl;
      —CO-Q, wherein Q is C₁₋₄alkoxyl, hydroxyl, NH₂ or NR_{c}R_{d}, wherein R_{c} and R_{d} are independently H or C₁₋₄alkyl;

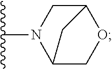 [SMILES: *N1CC2CC1CO2]

and
four to seven-membered heterocyclyl ring having one or two heteroatom ring members independently selected from O and N, wherein the heterocyclyl ring is optionally substituted with one to three halo substituents;
5) C₃₋₇cycloalkyl optionally substituted with one to three substituents independently selected from the group consisting of
   halo, hydroxyl;

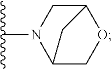 [SMILES: *N1CC2CC1CO2]

C₁₋₃alkyl optionally substituted with one to three substituents independently selected from the group consisting of halo, hydroxyl and C₁₋₃alkoxyl;
C₁₋₄alkoxyl optionally substituted with one to three substituents independently selected from the group consisting of halo, hydroxyl and C₁₋₃alkoxyl; and
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl;
6) C-linked 7-9 membered bridged cyclyl ring optionally having one or two heteroatom ring members independently selected from O and N, optionally substituted with one to three substituents independently selected from the group consisting of halo, hydroxyl, C₁₋₃alky and C₁₋₃alkoxyl;
7) C-linked 7-10 membered spirane cyclyl ring optionally having one or two heteroatom ring members independently selected from O and N, optionally substituted with one to three substituents independently selected from the group consisting of halo, hydroxyl, C₁₋₃alky and C₁₋₃alkoxyl; or
8) C-linked 6-9 membered fused cyclyl ring optionally having one or two heteroatom ring members independently selected from O and N, optionally substituted with one to three substituents independently selected from the group consisting of halo, hydroxyl, C₁₋₃alky and C₁₋₃alkoxyl;

R₄ and R₅, at each occurrence, are each independently selected from the group consisting of
H, halo, hydroxyl;
C₁₋₄alkyl optionally substituted with one to three substituents independently selected from the group consisting of halo, C₁₋₄alkoxy, OC₁₋₄haloalkyl, and four to seven-membered heterocyclyl ring having one or two heteroatom ring members independently selected from O and N;
C₃₋₆cycloalkyl optionally substituted with one to three substituents independently selected from the group consisting of halo, hydroxyl and C₁₋₄alkoxy;
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl; and
C₁₋₄alkoxyl optionally substituted with one to three substituents independently selected from the group consisting of halo, hydroxyl and C₁₋₄alkoxy.

Paragraph 2. The compound or a pharmaceutically acceptable salt thereof according to paragraph 1, wherein
X is CH or N;
n is 2, 3, 4 or 5;
A is O or NR^{a}, wherein R^{a} is H or C₁₋₄alkyl optionally substituted with C₁₋₃alkoxyl;
R₁ is H, halo, CN, C₁₋₄alkoxyl, C₂₋₆ alkenyl, C₂₋₆ alkynyl or C₁₋₄alkyl optionally substituted with one to three substituents independently selected from the group consisting of halo, hydroxyl and C₁₋₃alkoxyl;
R₂ is H, halo, CN, C₁₋₄haloalkyl, C₁₋₄alkyl optionally substituted with C₁₋₃alkoxyl and C₃₋₆cycloalkyl;
R₃ is
   1) H;
   2) —CO—Z, wherein Z is selected from the group consisting of
      C₃₋₆cycloalkyl and four to six-membered heterocyclyl ring having 0 as heteroatom ring member;
   3) four to six-membered heterocyclyl ring having one to two heteroatom ring members independently selected from O and N, and the heterocyclyl ring is optionally substituted with one to three substituents independently selected from the group consisting of
      halo;
      C₁₋₃alkyl optionally substituted with one to three substituents independently selected from halo, hydroxyl and C₁₋₃alkoxyl;
      C₁₋₃alkoxyl optionally substituted with one to three substituents independently selected from halo, hydroxyl and C₁₋₃alkoxyl; and
      four to six-membered heterocyclyl ring having one to two heteroatom ring members independently selected from O and N;
   4) C₁₋₄alkyl optionally substituted with one to three substituents independently selected from the group consisting of
      CN, hydroxyl, halo;
      C₁₋₂alkoxyl;
      —CO-Q, wherein Q is C₁₋₂alkoxyl, hydroxyl, or NH₂;

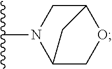 [SMILES: *N1CC2CC1CO2]

and
four to six-membered heterocyclyl ring having one to two heteroatom ring members independently selected from O and N, wherein the heterocyclyl ring is optionally substituted with one to three halo substituents;
5) C₃₋₆cycloalkyl optionally substituted with one to three substituents independently selected from the group consisting of
   halo, hydroxyl;

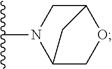 [SMILES: *N1CC2CC1CO2]

C₁₋₃alkyl optionally substituted with one to three substituents independently selected from halo, hydroxyl and C₁₋₃alkoxyl;
C₁₋₄alkoxyl optionally substituted with one to three substituents independently selected from halo, hydroxyl and C₁₋₃alkoxyl; and
four to six-membered heterocyclyl ring having one to two heteroatom ring members independently selected from O and N, wherein the heterocyclyl ring is optionally substituted with one to three halo substituents;
6) C-linked 7-9 membered bridged cyclyl ring optionally having one or two heteroatom ring members independently selected from O and N; or
7) C-linked 7-10 membered spirane cyclyl ring optionally having one or two heteroatom ring members independently selected from O and N;
R₄ and R₅, at each occurrence, are each independently selected from the group consisting of
   H, halo, hydroxyl;
   C₁₋₄alkyl optionally substituted with one to three substituents independently selected from halo and C₁₋₄alkoxy;
   C₃₋₆cycloalkyl; and
   C₁₋₄alkoxyl optionally substituted with one to three substituents independently selected from halo and C₁₋₄alkoxy.

Paragraph 3. The compound or a pharmaceutically acceptable salt thereof according to any of paragraphs 1 to 2, wherein R₁ is selected from the group consisting of H, halo, CN, methyl, methoxy, trifluoromethyl, ethenyl and ethynyl.

Paragraph 4. The compound or a pharmaceutically acceptable salt thereof according to any of paragraphs 1 to 3, wherein R₂ is selected from the group consisting of H, halo, CN, methyl, difluoromethyl, trifluoromethyl, cyclopropyl and methoxyethyl.

Paragraph 5. The compound or a pharmaceutically acceptable salt thereof according to any of paragraphs 1 to 4, wherein R₃ is
1) H;
2) —CO—Z, wherein Z is selected from the group consisting of cyclopropyl, oxetanyl and tetrahydro-2H-pyranyl;
3) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, wherein the heterocyclyl ring is optionally substituted with one to three substituents independently selected from the group consisting of
   hydroxyl, halo;
   C₁₋₃alkyl optionally substituted with one to three substituents independently selected from halo, hydroxyl and C₁₋₃alkoxyl;
   C₁₋₃alkoxyl optionally substituted with one to three substituents independently selected from halo, hydroxyl and C₁₋₃alkoxyl; and
   four to six-membered heterocyclyl ring having one to two heteroatom ring members independently selected from O and N;
4) C₁₋₄alkyl optionally substituted with one to three substituents independently selected from the group consisting of
   CN, hydroxyl, halo;
   C₁₋₂alkoxyl;
   —CO-Q, wherein Q is C₁₋₂alkoxyl, hydroxyl, or NH₂;

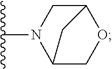 [SMILES: *N1CC2CC1CO2]

and
four to six-membered heterocyclyl ring selected from the group consisting of oxetanyl, tetrahydrofuranyl, tetrahydro-2H-pyranyl, pyrrolidinyl, piperidinyl and morpholinyl, wherein the heterocyclyl ring is optionally substituted with one to three halo substituents;
5) C₄₋₆ cycloalkyl optionally substituted with one to three substituents independently selected from the group consisting of
   halo, hydroxyl;
   C₁₋₃haloalkyl;

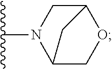 [SMILES: *N1CC2CC1CO2]

C₁₋₃alkyl optionally substituted with one to three substituents independently selected from halo, hydroxyl and C₁₋₃alkoxyl;
C₁₋₄alkoxyl optionally substituted with one to three substituents independently selected from halo, hydroxyl and C₁₋₃alkoxyl; and
four to six-membered heterocyclyl ring having one to two heteroatom ring members independently selected from O and N, wherein the heterocyclyl ring is optionally substituted with one to three halo substituents;

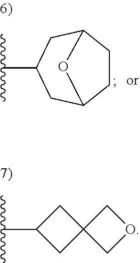 [SMILES: *C1CC2CCC(C1)O2 ; *C1CC2(COC2)C1]

Paragraph 6. The compound or a pharmaceutically acceptable salt thereof according to any of paragraphs 1 to 5, wherein R₃ is
1) H;
2) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, and the heterocyclyl ring is optionally substituted with one to three substituents independently selected from the group consisting of
   halo;
   oxetanyl; and
   C₁₋₃alkyl optionally substituted with one to three substituents independently selected from halo, hydroxyl and C₁₋₃alkoxyl;
3) 2-cyano-2-propyl;
4) C₄₋₆ cycloalkyl optionally substituted with one to three substituents independently selected from hydroxyl, morpholinyl and

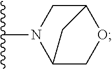 [SMILES: *N1CC2CC1CO2]

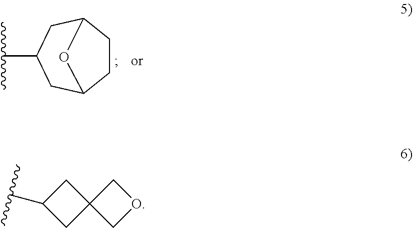 [SMILES: *C1CC2CCC(C1)O2 ; *C1CC2(COC2)C1]

Paragraph 7. The compound or a pharmaceutically acceptable salt thereof according to any of paragraphs 1 to 6, wherein R₄ and R₅, at each occurrence, are each independently selected from the group consisting of H, halo, C₁₋₃alkyl, methoxy, ethoxy, fluoromethyl, difluoromethyl, trifluoromethyl, difluoromethoxy, methoxymethyl and cyclopropyl.

Paragraph 8. The compound or a pharmaceutically acceptable salt thereof according to any of paragraphs 1 to 7, wherein R₄ and R₅, at each occurrence, are each independently selected from the group consisting of H, F, methyl, ethyl, methoxy, fluoromethyl, difluoromethyl, trifluoromethyl, and methoxymethyl.

Paragraph 9. The compound or a pharmaceutically acceptable salt thereof according to any of paragraphs 1 to 8, wherein A is NH, X is N and n is 3.

Paragraph 10. The compound according to any of paragraphs 1 to 9, having the structure of Formula (IA)

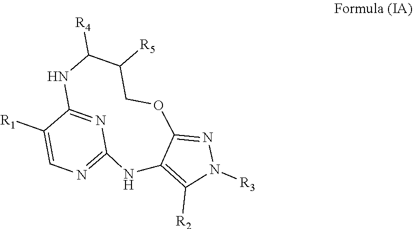

or a pharmaceutically acceptable salt thereof, wherein
R₁ is Br, Cl or CN;
R₂ is Cl, CN or methyl;
R₃ is
1) H
2) four to six-membered heterocyclyl ring selected from the group consisting of oxetanyl, azetidinyl, tetrahydrofuranyl, pyrrolidinyl, tetrahydro-2H-pyranyl, piperidinyl and morpholinyl, wherein the heterocyclyl ring is optionally substituted with one to three substituents independently selected from halo, methyl, methoxyethyl and oxytanyl;
3) 2-cyano-2-propyl;
4) C₄₋₆ cycloalkyl optionally substituted with one to three substituents independently selected from hydroxyl, morpholinyl and

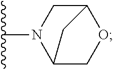 [SMILES: *N1CC2CC1CO2]

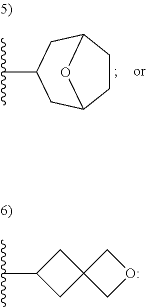

R₄ is H, methyl, ethyl, fluoromethyl, difluoromethyl, trifluoromethyl, or methoxymethyl; and
R₅ is H, F, methyl, or methoxy.

Paragraph 11. A compound selected from

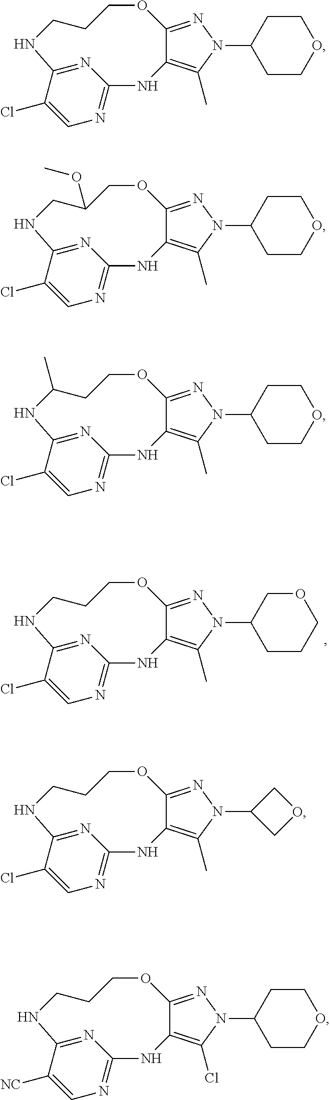 [SMILES: Cc1c2c(nn1C1CCOCC1)OCCCNc1nc(ncc1Cl)N2 ; COC1CNc2nc(ncc2Cl)Nc2c(nn(C3CCOCC3)c2C)OC1 ; Cc1c2c(nn1C1CCOCC1)OCCC(C)Nc1nc(ncc1Cl)N2 ; Cc1c2c(nn1C1CCCOC1)OCCCNc1nc(ncc1Cl)N2 ; Cc1c2c(nn1C1COC1)OCCCNc1nc(ncc1Cl)N2 ; N#Cc1cnc2nc1NCCCOc1nn(C3CCOCC3)c(Cl)c1N2]

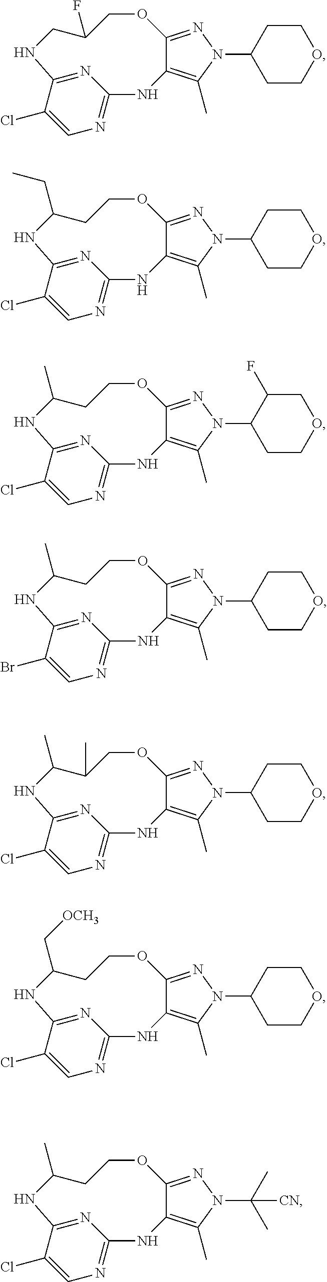

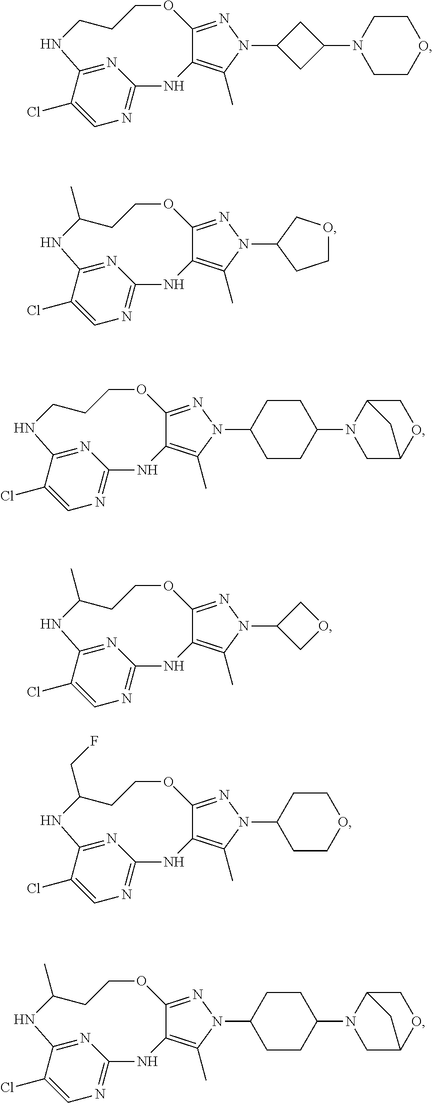

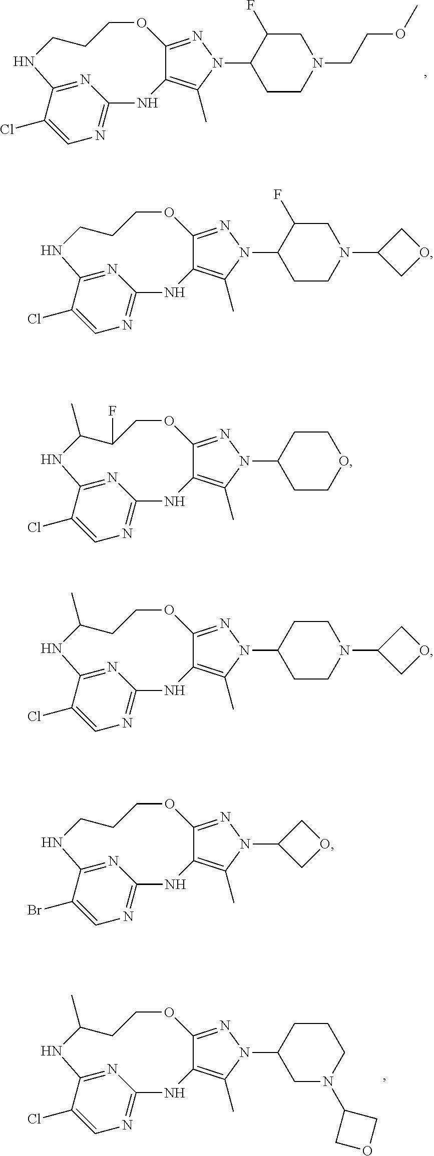 [SMILES: COCCN1CCC(n2nc3c(c2C)Nc2ncc(Cl)c(n2)NCCCO3)C(F)C1 ; Cc1c2c(nn1C1CCN(C3COC3)CC1F)OCCCNc1nc(ncc1Cl)N2 ; Cc1c2c(nn1C1CCOCC1)OCC(F)C(C)Nc1nc(ncc1Cl)N2 ; Cc1c2c(nn1C1CCN(C3COC3)CC1)OCCC(C)Nc1nc(ncc1Cl)N2 ; Cc1c2c(nn1C1COC1)OCCCNc1nc(ncc1Br)N2 ; Cc1c2c(nn1C1CCCN(C3COC3)C1)OCCC(C)Nc1nc(ncc1Cl)N2]

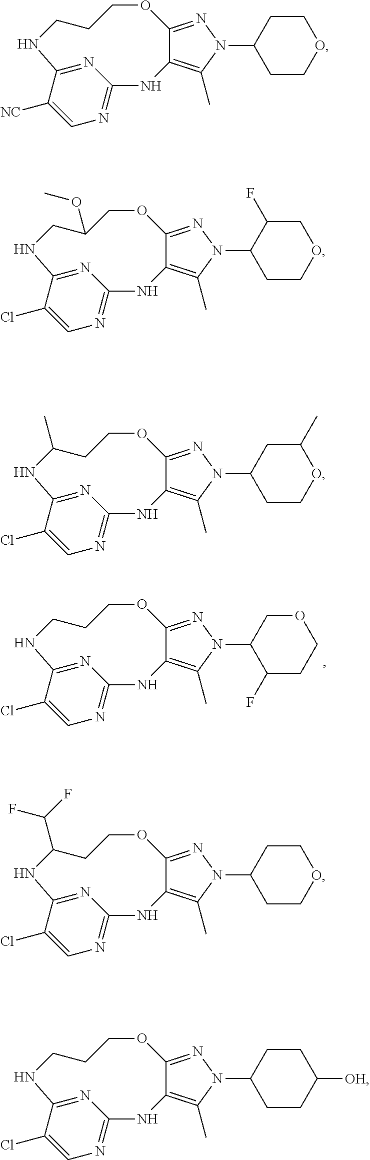 [SMILES: Cc1c2c(nn1C1CCOCC1)OCCCNc1nc(ncc1C#N)N2 ; COC1CNc2nc(ncc2Cl)Nc2c(nn(C3CCOCC3F)c2C)OC1 ; Cc1c2c(nn1C1CCOC(C)C1)OCCC(C)Nc1nc(ncc1Cl)N2 ; Cc1c2c(nn1C1COCCC1F)OCCCNc1nc(ncc1Cl)N2 ; Cc1c2c(nn1C1CCOCC1)OCCC(C(F)F)Nc1nc(ncc1Cl)N2 ; Cc1c2c(nn1C1CCC(O)CC1)OCCCNc1nc(ncc1Cl)N2]

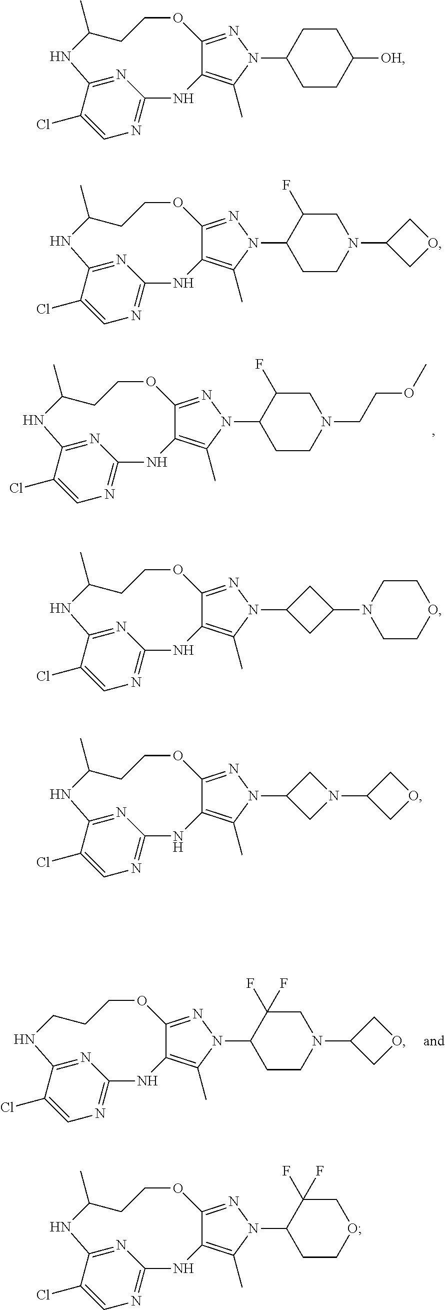

or a pharmaceutically acceptable salt thereof.

Paragraph 12. The compound or a pharmaceutically acceptable salt thereof according to any of paragraphs 1 to 10, wherein R₃ is H.

Paragraph 13. A compound selected from

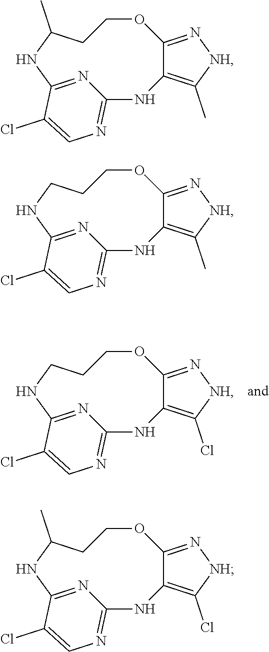

or a pharmaceutically acceptable salt thereof.

Paragraph 14. A compound of Formula (I), Formula (IA) or a pharmaceutically acceptable salt thereof according to any of paragraphs 1 to 13 for use in therapy.

Paragraph 15. A compound of Formula (I), Formula (IA) or a pharmaceutically acceptable salt thereof according to any of paragraphs 1 to 13 for use in the treatment of Parkinson's disease, Alzheimer's disease or amyotrophic lateral sclerosis (ALS).

Paragraph 16. A method of treatment of Parkinson's disease, Alzheimer's disease or amyotrophic lateral sclerosis (ALS), which comprises administering to a subject in need thereof a therapeutically effective amount of a compound of Formula (I), Formula (IA), or a pharmaceutically acceptable salt thereof according to any of paragraphs 1 to 13.

Paragraph 17. The method of paragraph 29, wherein the subject is a human.

Paragraph 18. Use of a compound of Formula (I), Formula (IA) or a pharmaceutically acceptable salt thereof according to any of paragraphs 1 to 13 in the manufacture of a medicament for the treatment of Parkinson's disease, Alzheimer's disease or amyotrophic lateral sclerosis (ALS).

Paragraph 19. A pharmaceutical composition comprising a compound of Formula (I), Formula (IA) or a pharmaceutically acceptable salt thereof according to any of paragraphs=s 1 to 13, and one pharmaceutically acceptable excipient.

Paragraph 20. A pharmaceutical composition for use in the treatment of Parkinson's disease, Alzheimer's disease or amyotrophic lateral sclerosis (ALS), wherein the composition comprises a compound of Formula (I), Formula (IA) or a pharmaceutically acceptable salt thereof according to any of paragraphs 1 to 13, and one pharmaceutically acceptable excipient.

### EXAMPLES

### General Experimental Procedures

The following descriptions and examples illustrate the invention. These examples are not intended to limit the scope of the present invention, but rather to provide guidance to the skilled artisans to prepare and use the compounds, compositions and methods of the present invention. While particular embodiments of the present invention are described, the skilled chemists will appreciate that various changes and modifications can be made without departing from the spirit and scope of the invention.

The chemical names of compounds described in the present application follows the principle of IUPAC nomenclature.

Heating of reaction mixtures with microwave irradiations was carried out on a Smith Creator (purchased from Personal Chemistry, Forboro/MA, now owned by Biotage), an Emrys Optimizer (purchased from Personal Chemistry) or an Explorer (provided by CEM Discover, Matthews/NC) microwave.

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the compounds in the examples may be carried out by conventional methods such as chromatography and/or re-crystallization using suitable solvents. Chromatographic methods are known to the skilled persons and include e.g. column chromatography, flash chromatography, HPLC (high performance liquid chromatography), and MDAP (mass directed auto-preparation, also referred to as mass directed LCMS purification). MDAP is described in e.g. W. Goetzinger et al, Int. J. Mass Spectrom. 2004, 238, 153-162.

Analtech Silica Gel GF and E. Merck Silica Gel 60 F-254 thin layer plates were used for thin layer chromatography (TLC). Both flash and gravity chromatography were carried out on E. Merck Kieselgel 60 (230-400 mesh) silica gel. Preparative HPLC were performed using a Gilson Preparative System using a Luna 5u C18(2) 100A reverse phase column eluting with a 10-80 gradient (0.1% aqueous TFA/acetonitrile) or a 10-80 gradient (water/acetonitrile). The CombiFlash system used for purification in this application was purchased from Isco, Inc. CombiFlash purification was carried out using a pre-packed SiO₂ column, a detector with UV wavelength at 254 nm and mixed solvents.

The terms “CombiFlash”, “Biotage®”, “Biotage 75” and “Biotage SP4®” when used herein refer to commercially available automated purification systems using pre-packed silica gel cartridges.

Title compounds were generally/typically characterized with LCMS or/and NMR. ¹H NMR or ¹⁹F NMR spectra were recorded using a Bruker Avance 400 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, td=triplets of doublets, dq=doublet of quartets, br=broad. J indicates the NMR coupling constant measured in Hertz (Hz).

All temperatures are reported in degrees Celsius (° C.). All other abbreviations are as described in the ACS Style Guide (American Chemical Society, Washington, D C, 1986).

Absolute stereochemistry can be determined by methods known to one skilled in the art, for example X-ray or Vibrational Circular Dichroism (VCD).

When an enantiomer or a diasteroisomer is described and the absolute stereochemistry of a chiral center is not known, the use of “*” at the chiral centre denotes that the absolute stereochemistry of the chiral center is not known, i.e. the compound as drawn may be either a single R enantiomer or a single S enantiomer. Where the absolute stereochemistry at a chiral center of an enantiomer or a diasteroisomer is known, a bold wedge symbol ( [symbol] ) or a hashed wedge symbol ( [symbol] ) is used as appropriate, without the use of “*” at the chiral centre.

### LCMS Conditions:

Instruments: HPLC: Agilent 1200 and MS: Agilent 6120

### 1) Acidic Conditions:

Column: Agilent SB-C18 4.6×30 mm-1.8 microns

Mobile phase: water containing 0.05% FA/0.05% CH₃CN

Detection: MS and photodiode array detector (PDA)

### 2) Basic Conditions:

### (A)

Column: XBridge™ C18 4.6×50 mm-3.5 microns

Mobile phase: water containing 10 mmol NH₄HCO₃/CH₃CN

Detection: MS and photodiode array detector (PDA)

### (B)

Column: Welch Ultimate XB-C18 5 μm 4.6*33 mm

Mobile phase: water containing 0.02% NH₄OAc/CH₃CN

Detection: MS and photodiode array detector (PDA)

MDAP Conditions:

### 1) Acidic Conditions:

Instrument: Waters instrument

Column: Sunfire Prep C18 column (5 um, 19×50 mm)

Mobile phase: water containing 0.05% TFA/acetonitrile.

### 2) Basic Conditions:

Instrument: Waters instrument

Column: Xbridge Prep C18 column (5 um, 19×50 mm)

Mobile phase: water containing 0.04% ammonia/acetonitrile.

### Prep-HPLC Conditions

Instrument: Waters instrument

Column: Xbridge Prep C18 column OBD (10 um, 19×250 mm)

Mobile phase: water containing 0.08% ammonia/acetonitrile.

### Chiral-HPLC Isolation Instruments:

1. Gilson Gx-281 Prep LC (Gilson 806 Manometric Module, Gilson 811D Dynamic Mixer, Gilson Gx-281 prep liquid handler, Gilson 306 Pump*2, Gilson 156 Detector),
2. Agilent 1200 series Prep LC (Agilent G1361A Prep pump*2, Agilent G2260A Prep ALS, Agilent G1315D DAD Detector, Agilent G1364B Prep FC),
3. Thar SFC Prep 80 (TharSFC ABPR1, TharSFC SFC Prep 80 CO₂ Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module).

### Chiral-HPLC Separation Conditions:

1. Chiral method A: AD-H, 0.46 cm I.D.×15 cm L; mobile Phase: heptane:ethanol (0.1% diethylamine)=60:40; flow rate: 0.5 mL/min; wavelength: 254 nm; temperature: 25° C.
2. Chiral method B: AS-H, 0.46 cm I.D.×15 cm L; mobile Phase: heptane: ethanol (0.1% diethylamine)=80:20; flow rate: 0.5 mL/min; Wavelength: 254 nm; Temperature: 25° C.
3. Chiral method C: OJ-H, 0.46 cm I.D.×15 cm L; mobile Phase: heptane: ethanol (0.1% diethylamine)=80:20; flow rate: 0.5 mL/min; Wavelength: 254 nm; Temperature: 25° C.
4. Chiral method D: ID-H, 0.46 cm I.D.×15 cm L; mobile Phase: heptane: i-PrOH (0.1% diethylamine)=80:20; flow rate: 0.5 mL/min; Wavelength: 254 nm; Temperature: 25° C.
5. Chiral method E: IC-H, 0.46 cm I.D.×15 cm L; mobile Phase: heptane: ethanol (0.1% diethylamine)=60:40; flow rate: 0.5 mL/min; wavelength: 254 nm; temperature: 25° C.
6. Chiral method F: OD-H, 0.46 cm I.D.×15 cm L; mobile Phase: heptane: ethanol (0.1% diethylamine)=60:40; flow rate: 0.5 mL/min; wavelength: 254 nm; temperature: 25° C.
7. Chiral method G: OZ-H, 0.46 cm I.D.×15 cm L; mobile Phase: heptane: ethanol (0.1% diethylamine)=60:40; flow rate: 0.5 mL/min; wavelength: 254 nm; temperature: 25° C.

### Abbreviations and Resource Sources

The following abbreviations and resources are used herein below:
atm—atmospheres
Ac—acetyl
Aq. —aqueous
Boc—tert-butyloxycarbonyl
Boc₂O—di-tert-butyl dicarbonate
Bn—benzyl
CAN—Ceric ammonium nitrate
conc.—concentrated
CyNMe₂—(CN)N(CH3)₂
DAST—N,N-diethylaminosulfur trifluoride
DCM—dichloromethane
DEAD—Diethyl azodicarboxylate
DHP—3,4-dihydro-2H-pyran
DIPEA—N, N-diisopropylethylamine
DIAD—diisopropyl azodicarboxylate
DMAP—4-Dimethylaminopyridine
DMF—N, N-dimethylformamide
DMSO—dimethyl sulfoxide
EA—ethyl acetate
Et—ethyl
EtOAc—ethyl acetate
EtOH—ethanol
Et₃N—triethylamine
FCC—flash column chromatography
HCl—hydrogen chloride
HOAc/AcOH—acetic acid
hrs—hours
HATU—1-[bis(dimethylamino)methylene]-1H-1,2,3-triazolo[4,5-b]pyridinium3-oxidhexafluoro phosphate
IBX—2-iodoxybenzoic acid
i-PrOH—isopropyl alcohol
LDA—lithium diisopropylamide
LiHMDS—lithium bis(trimethylsilyl)amide
LiAlH₄—lithium aluminium hydride
mCBPA—meta-Chloroperoxybenzoic acid
Me—methyl
MeOH—methanol
CH₃OH—methanol
Ms—methane sulfonate
MsCl—methanesulfonyl chloride
NMM—4-methylmorpholine
NMP—1-methyl-2-pyrrolidone
n-Bu—n-butyl
n-BuLi—n-Butyllithium
t-Bu—t-butyl
Pd₂(dba)₃—tris(dibenzylideneacetone)dipalladium(0)
Pd(dppf)Cl₂-[1,1′-Bis(diphenylphosphino)ferrocene]palladium(II) dichloride
PE—petroleum ether
Ph₃P/PPh₃—triphenylphosphine
PMB—para methoxy benzyl
POCl₃—Phosphoryl chloride
p-TsOH—p-toluenesulfonic acid
rt—room temperature
RT—retention time
Ru-phos—2-dicyclohexylphosphino-2′,6′-diisopropoxybiphenyl
sat. —saturated
SEM—2-(trimethylsilyl)ethoxymethyl
SEMCl—2-(trimethylsilyl)ethoxymethyl chloride
SFC—Supercritical fluid chromatography
TBAI—tetrabutylammonium iodide
TBME—tert-butyl methyl ether
TEA—triethylamine
TFA—trifluoroacetic acid
TFAA—Trifluoroacetic anhydride
THF—tetrahydrofuran
THP—tetrahydropyranyl
TLC—thin layer chromatography
TMSCF₃—trifluoromethyltrimethylsilane
Tween 20—polysorbate 20

### Description D1

### 1-(Tetrahydro-2H-pyran-4-yl)-1H-pyrazole (D1)

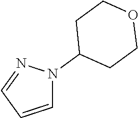 [SMILES: c1cnn(C2CCOCC2)c1]

A mixture of 1H-pyrazole (10.5 g, 154.23 mmol), tetrahydro-2H-pyran-4-yl methanesulfonate (41.7 g, 231.35 mmol) and Cs₂CO₃ (75.4 g, 231.35 mmol) in DMF (600 mL) was stirred at 100° C. for 24 hrs. The reaction was filtered and the filtrate was diluted with EtOAc (1000 mL). The mixture was washed with brine (6×200 mL), dried over anhydrous Na₂SO₄, filtered and concentrated. The crude was purified by C18 column chromatography (CH₃CN:H₂O=30:70) to give the title compound as an off-white solid (12.6 g, yield 53.7%). ¹H NMR (400 MHz, CDCl₃): 7.52 (br, 1H), 7.44 (br, 1H), 6.27 (br, 1H), 4.41-4.33 (m, 1H), 4.12˜4.10 (m, 2H), 3.58˜3.52 (m, 2H), 2.11˜2.03 (m, 4H).

### Description D2

### 5-Methyl-1-(tetrahydro-2H-pyran-4-yl)-1H-pyrazole (D2)

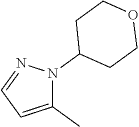

To a solution of D1 (8.5 g, 55.85 mmol) in THF (100 mL) was added n-BuLi (1.6 M in THF, 53 mL, 83.76 mmol) at −78° C. under argon for 30 min. Then CH₃I (15.8 g, 111.70 mmol) was added drop wise. The reaction was stirred at −78° C. for 30 min. The mixture was poured into ice water (150 mL) and extracted with EtOAc (2×100 mL). The combined organic layers were dried over anhydrous Na₂SO₄, filtered and concentrated. The crude was purified by C18 column chromatography (CH₃CN:H₂O=40:60) to give the title compound as an off-white solid (5.7 g, yield 61.4%). LC-MS: 167.2 [M+H]+.

### Description D3

### 3, 4-Dibromo-5-methyl-1-(tetrahydro-2H-pyran-4-yl)-1H-pyrazole (D3)

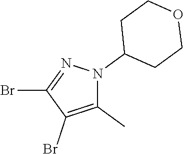

To a solution of D2 (5.7 g, 34.29 mmol) and sodium acetate (5.7 g, 68.58 mmol) in AcOH (50 mL) was added Br₂ (17.6 mL, 342.92 mmol). The reaction was stirred at 85° C. for 16 hrs. Sat. Na₂SO₃ (200 mL) was added and the mixture was stirred at room temperature for 1 hour. Then the mixture was extracted with EtOAc (2×150 mL) and the combined organic layers were washed with brine (2×100 mL), dried over anhydrous Na₂SO₄, filtered and concentrated. Then the residue was washed with hexane to give the title compound as a yellow solid (5.407 g, yield 48.7%). LC-MS: 324.9 [M+H]+.

### Description D4

### 3-Bromo-5-methyl-4-nitro-1-(tetrahydro-2H-pyran-4-yl)-1H-pyrazole (D4)

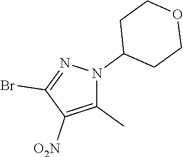 [SMILES: Cc1c([N+](=O)[O-])c(Br)nn1C1CCOCC1]

To a solution of D3 (5.407 g, 16.69 mmol) in 95% H₂SO₄ (50 mL) at 0° C. was added 97% HNO₃ (50 mL) dropwise. The reaction was stirred at 0° C. for 0.5 hour, then slowly heated to room temperature and maintained for 1 hour. The mixture was poured into ice water (300 mL) and extracted with EtOAc (200 mL). The organic layer was washed with H₂O (2×50 mL), dried over anhydrous Na₂SO₄, filtered and concentrated to give the title compound as an off-white solid (1.127 g, yield 23.3%).

### Description D5

### 5-Methyl-4-nitro-1-(tetrahydro-2H-pyran-4-yl)-1H-pyrazol-3-ol (D5)

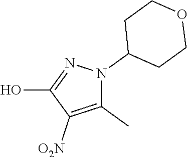 [SMILES: Cc1c([N+](=O)[O-])c(O)nn1C1CCOCC1]

To a mixture of D4 (560 mg, 1.93 mmol) in H₂O (20 mL) was added KOH (2.24 g). The reaction was stirred at 100° C. for 18 hrs. The mixture was washed with EtOAc (2×10 mL). The aqueous phase was neutralized with 2N HCl to pH=3 and extracted with DCM (2×20 mL). The combined organic layers were dried over anhydrous Na₂SO₄, filtered and concentrated to give the title compound as a yellow solid (380 mg, yield 86.6%).

### Description D6

### tert-Butyl-(3-((5-methyl-4-nitro-1-(tetrahydro-2H-pyran-4-yl)-1H-pyrazol-3-yl)oxy) propyl) carbamate (D6)

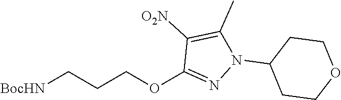 [SMILES: Cc1c([N+](=O)[O-])c(OCCCNC(=O)OC(C)(C)C)nn1C1CCOCC1]

A mixture of D5 (120 mg, 0.53 mmol), tert-butyl (3-bromopropyl)carbamate (187 mg, 0.79 mmol) and Cs₂CO₃ (346 mg, 1.06 mmol) in DMF (10 mL) was heated at 80° C. for 16 hrs. The mixture was poured into water (50 mL) and extracted with EtOAc (2×30 mL). The combined organic layer was washed with brine (5×30 mL), dried over anhydrous Na₂SO₄, filtered and concentrated to give the title compound, which was used for next step without further purification. LC-MS: 285 [M+H−100]⁺.

### Description D7

### 3-((5-Methyl-4-nitro-1-(tetrahydro-2H-pyran-4-yl)-1H-pyrazol-3-yl)oxy)propan-1-amine, HCl salt (D7)

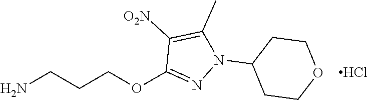 [SMILES: Cc1c([N+](=O)[O-])c(OCCCN)nn1C1CCOCC1 ; Cl]

A solution of D6 (204 mg, 0.53 mmol) in 4M HCl/MeOH (15 mL) was heated to 35° C. and stirred for 2 hrs. The reaction was concentrated to give the title compound, which was used in next step without further purification. LC-MS: 285.3 [M+H]⁺.

### Description D8

### 2,5-Dichloro-N-(3-((5-methyl-4-nitro-1-(tetrahydro-2H-pyran-4-yl)-1H-pyrazol-3-yl)oxy)propyl)pyrimidin-4-amine (D8)

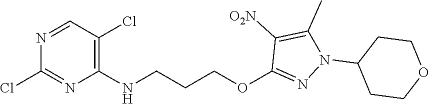 [SMILES: Cc1c([N+](=O)[O-])c(OCCCNc2nc(Cl)ncc2Cl)nn1C1CCOCC1]

To a solution of D7 (151 mg, 0.53 mmol) in DMF (20 mL) was added Et₃N (0.4 mL, 2.65 mmol) at room temperature and 2,4,5-trichloropyrimidine (138 mg, 0.75 mmol) was then added drop wise. The reaction was stirred at room temperature for 10 min. The mixture was poured into water (50 mL) and extracted with EtOAc (2×30 mL). The combined organic layers were washed with brine (5×20 mL), dried over anhydrous Na₂SO₄, filtered and concentrated. The crude was purified by column chromatography on silica gel (PE:EtOAc=2:1) to give the title compound as a yellow solid (154 mg, yield 67.6%). LC-MS: 431.3 [M+H]⁺.

### Description D9

### N-(3-((4-Amino-5-methyl-1-(tetrahydro-2H-pyran-4-yl)-1H-pyrazol-3-yl)oxy)propyl)-2,5-dichloropyrimidin-4-amine (D9)

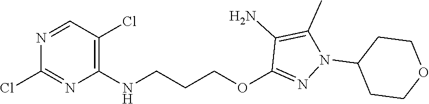 [SMILES: Cc1c(N)c(OCCCNc2nc(Cl)ncc2Cl)nn1C1CCOCC1]

To a solution of D8 (154 mg, 0.36 mmol) in EtOH/H₂O (20 mL/5 mL) were added NH₄Cl (96 mg, 1.79 mmol) and Fe (102 mg, 1.79 mmol). The reaction was stirred at 80° C. for 2 hrs. The mixture was filtered and the filtrate was concentrated. The residue was diluted with EtOAc (30 mL) and washed with H₂O (2×20 mL). The organic layer was dried over anhydrous Na₂SO₄, filtered and concentrated to give the title compound as a yellow solid (121 mg, yield 84.4%). LC-MS: 401.3 [M+H]⁺.

### Description D10

### tert-Butyl benzyl(3-(benzyloxy)-2-hydroxypropyl)carbamate (D10)

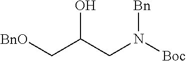 [SMILES: CC(C)(C)OC(=O)N(Cc1ccccc1)CC(O)COCc1ccccc1]

To a solution of BnNH₂ (69.4 g, 0.65 mol) in 2% Tween 20/water (1 L) was added 2-((benzyloxy)methyl)oxirane (90 g, 0.54 mol) at 5˜10° C. dropwise. The reaction was stirred overnight at room temperature. To this mixture was added Boc₂O (164.8 g, 0.756 mol) at 0-5° C. dropwise. The reaction was stirred overnight at room temperature. CH₂Cl₂ was added and the organic layer was washed with brine (500 mL), dried over anhydrous Na₂SO₄ and concentrated. The crude was purified by flash column chromatography on silica gel (PE:EtOAc=10:1) to give the title compound as yellow oil (90 g, yield 45%). LC-MS: 394.3 [M+Na]⁺.

### Description D11

### tert-Butyl benzyl (3-(benzyloxy)-2-methoxypropyl) carbamate (D11)

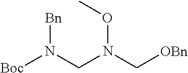 [SMILES: CON(COCc1ccccc1)CN(Cc1ccccc1)C(=O)OC(C)(C)C]

To a solution of D10 (5.9 g, 15.9 mmol) in DMF (10 mL) were added CH₃I (6.78 g, 47.8 mmol) and NaH (0.69 g, 17.4 mmol, 60%) at 0˜5° C. The reaction was stirred at 0˜5° C. for 3 hrs. The mixture was poured into water (100 mL) and extracted with EtOAc (100 mL). The combined organic layer was washed with brine, dried over anhydrous Na₂SO₄ and concentrated. The crude was purified by column chromatography on silica gel (PE:EtOAc=3:1) to give the title compound as yellow oil (3.6 g, yield 59%). LC-MS: 386.3 [M+H]⁺.

### Description D12

### tert-Butyl (3-hydroxy-2-methoxypropyl)carbamate (D12)

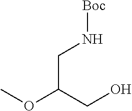

A solution of D11 (3.6 g) and Pd/C (1 g) in MeOH (40 mL) was stirred at 50° C. under hydrogen overnight. The mixture was filtered, the filter cake was washed with MeOH (2×20 mL) and the combined filtrate was concentrated. The crude was purified by column chromatography on silica gel (PE:EtOAc=3:1) to give the title compound as oil (318 mg, yield 16.6%).

### Description D13

### 3-((tert-Butoxycarbonyl)amino)-2-methoxypropyl methanesulfonate (D13)

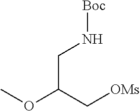 [SMILES: COC(CNC(=O)OC(C)(C)C)COS(C)(=O)=O]

To a solution of D12 (318 mg, 1.55 mmol) in CH₂Cl₂(20 mL) were added Et₃N (470 mg, 4.65 mmol) and after stirred for 30 min, MsCl (266.5 mg, 2.32 mmol) was added at 0˜5° C. The reaction was stirred at 25° C. for 3 hrs. The mixture was poured into water (50 mL) and extracted with EtOAc (2×50 mL). The combined organic layer was washed with brine, dried over anhydrous Na₂SO₄ and concentrated to give the title compound as yellow oil (400 mg, yield 91%). LC-MS: 306.2 [M+Na]⁺.

### Description D14

### tert-Butyl (2-methoxy-3-((5-methyl-4-nitro-1-(tetrahydro-2H-pyran-4-yl)-1H-pyrazol-3-yl)oxy)propyl)carbamate (D14)

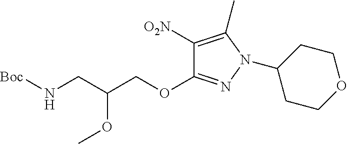 [SMILES: COC(CNC(=O)OC(C)(C)C)COc1nn(C2CCOCC2)c(C)c1[N+](=O)[O-]]

To a solution of D13 (350 mg, 1.23 mmol) in DMF (20 mL) were added K₂CO₃ (256 mg, 1.85 mmol) and D5 (141 mg, 0.61 mmol). The reaction was stirred at 80° C. overnight. The mixture was poured into water (100 mL) and extracted with EtOAc (2×80 mL). The organic layer was washed with brine, dried over anhydrous Na₂SO₄ and concentrated. The crude was purified by column chromatography on silica gel (PE:EtOAc=10:1) to give the title compound as yellow oil (130 mg, yield 50%). LC-MS: 437.3 [M+Na]⁺.

### Description D15

### 2-Methoxy-3-((5-methyl-4-nitro-1-(tetrahydro-2H-pyran-4-yl)-1H-pyrazol-3-yl)oxy) propan-1-amine, TFA salt (D15)

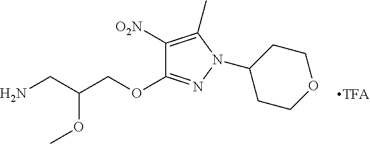 [SMILES: COC(CN)COc1nn(C2CCOCC2)c(C)c1[N+](=O)[O-]]

To a solution of D14 (130 mg) in CH₂Cl₂ (20 mL) was added TFA (4 mL). The reaction was stirred at room temperature for 1 hour. The mixture was concentrated to give the title compound as yellow oil (150 mg, crude). LC-MS: 316.3 [M+H]⁺.

### Description D16

### 2,5-Dichloro-N-(2-methoxy-3-((5-methyl-4-nitro-1-(tetrahydro-2H-pyran-4-yl)-1H-pyrazol-3-yl)oxy)propyl)pyrimidin-4-amine (D16)

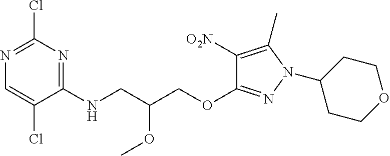 [SMILES: COC(CNc1nc(Cl)ncc1Cl)COc1nn(C2CCOCC2)c(C)c1[N+](=O)[O-]]

To a solution of D15 (97 mg, 0.31 mmol) in 2% Tween/water (2%, 10 mL) were added Et₃N (1 mL) and 2,4,5-trichloropyrimidine (57.4 mg, 0.31 mmol). The reaction was stirred at room temperature for 1 hour. The suspension was filtered and the yellow solid was obtained (120 mg, yield 93.7%). LC-MS: 461.1 [M+H]⁺.

### Description D17

### 3-((2,5-Dichloropyrimidin-4-yl)amino)butan-1-ol (D17)

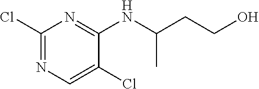

To a solution of 2,4,5-trichloropyrimidine (1.83 g, 10 mmol) in 2% Tween 20/water (50 mL) was added 3-aminobutan-1-ol (880 mg, 10 mmol) at 0° C. and stirred for 4 hrs. The reaction was concentrated and filtered. The solid was washed with CAN to give the title compound as a white solid (1.5 g, yield 64%). LC-MS: 236.1 [M+H]⁺.

### Description D18

### 3-((2,5-Dichloropyrimidin-4-yl)amino)butyl methanesulfonate (D18)

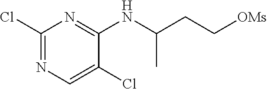 [SMILES: CC(CCOS(C)(=O)=O)Nc1nc(Cl)ncc1Cl]

To a solution of D17 (1.5 g, 6.4 mmol) in CH₂Cl₂ (20 mL) were added Et₃N (3 mL) and MsCl (2.6 g, 12.7 mmol). The reaction was stirred at room temperature for 3 hrs. The mixture was concentrated and the residue was poured into water and extracted with CH₂Cl₂. The organic layer was concentrated to give the title compound as a yellow solid (1.8 g, yield 90%). LC-MS: 314.1 [M+H]⁺.

### Description D19

### 2,5-Dichloro-N-(4-((5-methyl-4-nitro-1-(tetrahydro-2H-pyran-4-yl)-1H-pyrazol-3-yl)oxy)butan-2-yl)pyrimidin-4-amine (D19)

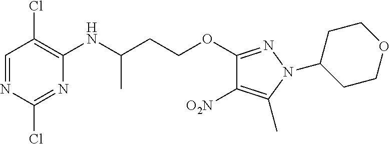 [SMILES: Cc1c([N+](=O)[O-])c(OCCC(C)Nc2nc(Cl)ncc2Cl)nn1C1CCOCC1]

To a solution of D18 (1.0 g, 2.46 mmol) in DMF (40 mL) were added K₂CO₃ (340 mg, 2.46 mmoL) and D5 (190 mg, 0.82 mmol). The reaction was stirred at 80° C. overnight. The mixture was poured into water and extracted with EtOAc (3×100 mL). The combined organic layers were washed with brine, dried over anhydrous Na₂SO₄ and concentrated. The crude was purified by column chromatography on silica gel (PE:EtOAc=3:1) to give the title compound as a yellow solid (250 mg, yield 68%). LC-MS: 445.1 [M+H]⁺.

### Description D20

### 3,4,5-Tribromo-1-(tetrahydro-2H-pyran-3-yl)-1H-pyrazol (D20)

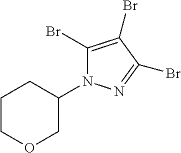 [SMILES: Brc1nn(C2CCCOC2)c(Br)c1Br]

To a solution of 3,4,5-tribromo-1H-pyrazole (21 g, 69 mmol) in THF (200 mL) were added tetrahydro-2H-pyran-3-ol (8.5 g, 83 mmol), PPh₃(36 g, 138 mmol), DIAD (27 g, 138 mmol) at 0° C. The resulting mixture was stirred at 0° C. for 3 hrs. The mixture was poured into water (100 mL) and extracted with EtOAc (3×100 mL). The combined organic layers were washed with brine, dried over anhydrous Na₂SO₄ and concentrated. The crude was purified by column chromatography on silica gel (PE:EtOAc=10:1) to give the title compound as oil (10 g, yield 50%).

### Description D21 and D22

### 3,4-Dibromo-5-methyl-1-(tetrahydro-2H-pyran-3-yl)-1H-pyrazole (D21)

### 3,4-Dibromo-1-(tetrahydro-2H-pyran-3-yl)-1H-pyrazole (D22)

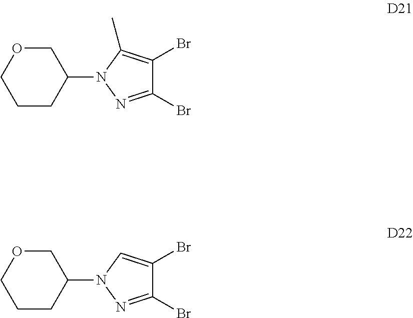

To a solution of D20 (1.0 g, 2.6 mmol) in THF (10 mL) was added n-BuLi (1.6 M in THF, 1.6 mL, 2.6 mmol) at −70° C. under argon for 30 min, and then CH₃I (0.8 g, 5.2 mmol) was added drop wise. The reaction was stirred at −70° C. for 2 hrs. The mixture was poured into ice and the sat. NH₄Cl solution and extracted with EtOAc (2×100 mL). The combined organic layer was washed with brine, dried over anhydrous Na₂SO₄ and concentrated. The crude was purified by column chromatography on silica gel (PE:EtOAc=10:1) to give the mixture of the title compounds as yellow solids (900 mg, yield 60%). D21: LC-MS: 324.9 [M+H]⁺. D22: LC-MS: 310.9 [M+H]⁺.

### Description D23 and D24

### 3-Bromo-5-methyl-4-nitro-1-(tetrahydro-2H-pyran-3-yl)-1H-pyrazole (D23)

### 3-Bromo-4-nitro-1-(tetrahydro-2H-pyran-3-yl)-1H-pyrazole (D24)

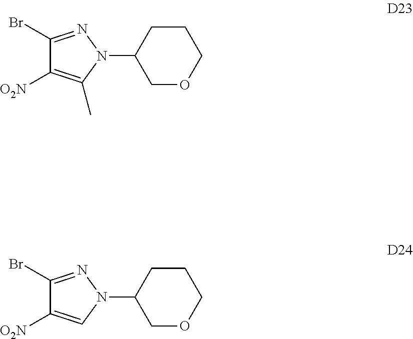 [SMILES: Cc1c([N+](=O)[O-])c(Br)nn1C1CCCOC1 ; O=[N+]([O-])c1cn(C2CCCOC2)nc1Br]

To a stirred solution of D21 and D22 (900 mg, 2.8 mmol) in 95% H₂SO₄ (10 mL) at 0° C. was added 97% HNO₃ (9 mL) dropwise. The mixture was stirred at 0° C. for 3 hrs. The reaction mixture was poured into ice, and extracted with EtOAc (3×50 mL). The combined organic layers were washed with brine, dried over anhydrous Na₂SO₄ and concentrated in vaxuo. The crude product was purified by column chromatography on silica gel (PE:EtOAc=10:1) to give the mixture of the title compounds as yellow solids (800 mg, yield 90%). D23: LC-MS: 290.1 [M+H]⁺. D24: LC-MS: 276.0 [M+H]⁺.

### Description D25 and D26

### 5-Methyl-4-nitro-1-(tetrahydro-2H-pyran-3-yl)-1H-pyrazol-3-ol (D25)

### 4-Nitro-1-(tetrahydro-2H-pyran-3-yl)-1H-pyrazol-3-ol (D26)

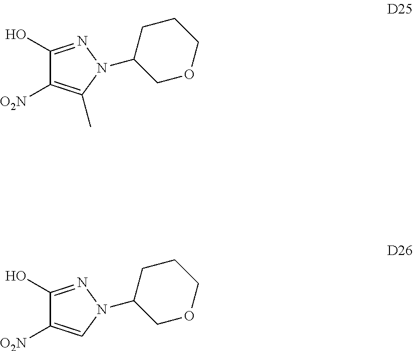 [SMILES: Cc1c([N+](=O)[O-])c(O)nn1C1CCCOC1 ; O=[N+]([O-])c1cn(C2CCCOC2)nc1O]

A mixture of D23, D24 (400 mg, 1.4 mmol) and KOH (1.2 g) in H₂O (10 mL) was stirred at 120° C. overnight. The mixture was washed with EtOAc (2×20 mL). The aqueous neutralized with 6N HCl to pH=3 and extracted with EtOAc (2×20 mL). The combined organic layers were washed with brine, dried over anhydrous Na₂SO₄ and concentrated in vacuo to give the mixture of the title compounds as yellow solids (200 mg, yield 56%).

### Description D27

### N-(3-Bromopropyl)-2,5-dichloropyrimidin-4-amine (D27)

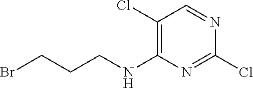 [SMILES: Clc1ncc(Cl)c(NCCCBr)n1]

To a solution of 3-bromopropan-1-amine hydrobromide (5.6 g, 25 mmol) in i-PrOH (40 mL) were added Et₃N (7.74 g, 76 mmol) and 2,4,5-trichloropyrimidine (4.69 g, 25 mmol) at 0° C. The reaction was stirred at room temperature overnight. The mixture was poured into water (100 mL) and extracted with EtOAc (100 mL). The combined organic layer was washed with brine, dried over anhydrous Na₂SO₄ and concentrated. The crude was purified by column chromatography on silica gel (PE:EtOAc=10:1) to give the title compound as white solid (4 g, 55% yield). LC-MS: 286.1 [M+H]⁺.

### Description D28 and D29

### 2,5-Dichloro-N-(3-((5-methyl-4-nitro-1-(tetrahydro-2H-pyran-3-yl)-1H-pyrazol-3-yl) oxy)propyl)pyrimidin-4-amine (D28)

### 2,5-Dichloro-N-(3-((4-nitro-1-(tetrahydro-2H-pyran-3-yl)-1H-pyrazol-3-yl)oxy)propyl) pyrimidin-4-amine (D29)

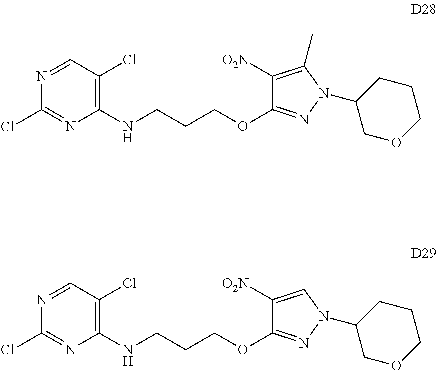 [SMILES: Cc1c([N+](=O)[O-])c(OCCCNc2nc(Cl)ncc2Cl)nn1C1CCCOC1 ; O=[N+]([O-])c1cn(C2CCCOC2)nc1OCCCNc1nc(Cl)ncc1Cl]

A solution of D25 and D26 (500 mg, 2.2 mmol), D27 (753 mg, 2.6 mmol) and K₂CO₃ (607 mg, 4.4 mmol) in DMF (5 mL) was stirred at 80° C. for 16 hrs. The mixture was poured into H₂O (10 mL) and extracted with EtOAc (3×10 mL). The combined organic layers were washed with brine, dried over anhydrous Na₂SO₄ and concentrated. The crude was purified by column chromatography on silica gel (PE:EtOAc=1:1) to give the mixture of title compounds as yellow solid (500 mg, yield 58%).

### Description D30

### 5-Chloro-3-methyl-4-nitro-1-((2-(trimethylsilyl)ethoxy)methyl)-1H-pyrazole (D30)

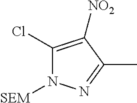 [SMILES: Cc1nn(COCC[Si](C)(C)C)c(Cl)c1[N+](=O)[O-]]

To a solution of 5-chloro-3-methyl-4-nitro-1H-pyrazole (12.5 g, 77.4 mmol) and Cy₂NMe (21.2 g/23.2 mL, 108.5 mmol) in THF (230 mL) under argon at 0° C. was added SEMCl (15.5 g/16.5 mL, 93.0 mmol) via syringe over 5 minutes. The reaction was stirred under argon at 0° C. for 15 min and then at room temperature for 1 hour. The mixture was diluted with water (200 mL), followed by addition of brine (100 mL). The resultant was extracted with EtOAc (2×150 mL). The combined organics were washed with brine (200 mL), dried over anhydrous Na₂SO₄, filtered and concentrated. The crude was purified by column chromatography on silica gel (PE:EtOAc=10:1) to give the title compound as light yellow oil (11.5 g, yield 50%).

### Description D31

### tert-Butyl (3-((3-methyl-4-nitro-1-((2-(trimethylsilyl)ethoxy)methyl)-1H-pyrazol-5-yl)oxy) propyl)carbamate (D31)

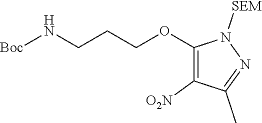 [SMILES: Cc1nn(COCC[Si](C)(C)C)c(OCCCNC(=O)OC(C)(C)C)c1[N+](=O)[O-]]

A mixture of D30 (22.9 g, 77.4 mmol) and N-Boc-propanolamine (27.1 g, 154.7 mmol) was dried under vacuo for 15 minutes. Dry toluene (390 mL) was added under argon, followed by sequential addition of CuI (2.21 g, 11.6 mmol), 1,10-phenanthroline (4.18 g, 23.2 mmol) and Cs₂CO₃ (40.4 g, 124 mmol). The reaction was stirred overnight under argon at 90° C. The cooled mixture was filtered through a pad of Celite and the filter cake was washed by EtOAc (1 L). The combined filtrate was concentrated to dryness and the dark residue oil was purified by chromatography on silica gel (PE:EtOAc=15:1 to 5:1) to give the title compound as orange oil (11.8 g, yield 35%). LC-MS: 453.3 [M+Na]⁺. ¹H NMR (400 MHz, CDCl₃): δ 5.29 (s, 2H), 5.10 (br, 1H), 4.43 (t, J=5.8 Hz, 2H), 3.66 (t, J=8.4 Hz, 2H), 3.39˜3.37 (m, 2H), 2.50 (s, 3H), 2.06˜2.02 (m, 2H), 1.44 (s, 9H), 0.94 (t, J=8.4 Hz, 2H), 0.00 (s, 9H).

### Description D32

### 3-((5-Methyl-4-nitro-1H-pyrazol-3-yl)oxy)propan-1-amine, HCl salt (D32)

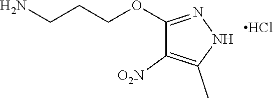

To a solution of D31 (11.8 g, 27.4 mmol) in MeOH (20 mL) under argon at room temperature was added HCl/MeOH (5 M, 240 mL). The reaction was stirred overnight under argon at 40° C. The light yellow suspension was concentrated to dryness. The residue was suspended in MeOH (30 mL), stirred for 15 minutes, filtered. The filter cake was collected and dried under vacuo to give the title compound as a light yellow solid (5.43 g, yield 83%). ¹H NMR (400 MHz, DMSO-d₆): δ 13.22 (s, 1H), 8.02 (br, 3H), 4.32 (t, J=6.0 Hz, 2H), 2.95 (br, 2H), 2.50 (s, 3H), 2.09˜2.03 (m, 2H).

### Description D33

### 2,5-Dichloro-N-(3-((5-methyl-4-nitro-1H-pyrazol-3-yl)oxy)propyl)pyrimidin-4-amine (D33)

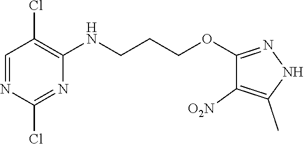 [SMILES: Cc1[nH]nc(OCCCNc2nc(Cl)ncc2Cl)c1[N+](=O)[O-]]

To a suspension of D32 (4.87 g, 20.6 mmol) in i-PrOH (150 mL) was added DIPEA (13.6 mL, 82.3 mmol). The reaction was stirred under argon at 0° C. for 10 minutes and 2,4,5-trichloropyrimidine (4.15 g, 22.6 mmol) was added via syringe. The mixture was allowed to warm to room temperature and stirred overnight under argon. The mixture was concentrated to dryness. The residue was suspended in water (100 mL), filtered. The filter cake was washed by water (100 mL), dried under infrared lamp at 50° C. for 2 hrs and collected to give the title compound as an off-white solid (7.08 g, yield 99%). LC-MS: 347.2 [M+H]⁺. ¹H NMR (400 MHz, DMSO-d₆): δ 12.90 (br, 1H), 8.14 (s, 1H), 7.98 (br, 1H), 4.28 (t, J=5.6 Hz, 2H), 3.56˜3.51 (m, 2H), 2.47 (s, 3H), 2.05 (t, J=6.2 Hz, 2H).

### Description D34

### 2,5-Dichloro-N-(3-((5-methyl-4-nitro-1-(oxetan-3-yl)-1H-pyrazol-3-yl)oxy)propyl) pyrimidin-4-amine (D34)

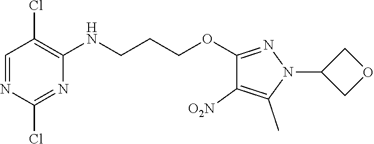 [SMILES: Cc1c([N+](=O)[O-])c(OCCCNc2nc(Cl)ncc2Cl)nn1C1COC1]

To a solution of D33 (200 mg, 0.576 mmol) and oxetan-3-yl 4-methylbenzenesulfonate (263 mg, 1.15 mmol) in DMF (3.0 mL) under argon at room temperature was added Cs₂CO₃ (563 mg, 1.73 mmol). The reaction was stirred under argon at 90° C. overnight. The mixture was diluted with water (25 mL), extracted with EtOAc (3×25 mL). The combined organics were washed with brine (20 mL), dried over anhydrous Na₂SO₄, filtered. The filtrate was concentrated and the crude was purified by prep-TLC (CH₂Cl₂:EtOAc=3:1) to give the title compound as a white solid (50 mg, yield 21%). ¹H NMR (400 MHz, CDCl₃): δ 8.00 (s, 1H), 5.98 (s, 1H), 5.40 (t, J=7.0 Hz, 1H), 5.15 (t, J=6.2 Hz, 2H), 4.94 (t, J=6.8 Hz, 2H), 4.54 (t, J=5.4 Hz, 2H), 3.79 (dd, J=11.8, 5.4 Hz, 2H), 2.58 (s, 3H), 2.26˜2.20 (m, 2H).

### Description D35

### 3-Chloro-4-nitro-1-(tetrahydro-2H-pyran-4-yl)-1H-pyrazole (D35)

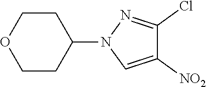 [SMILES: O=[N+]([O-])c1cn(C2CCOCC2)nc1Cl]

A mixture of 5-chloro-4-nitro-1H-pyrazole (12.0 g, 81.34 mmol), tetrahydro-2H-pyran-4-yl methanesulfonate (14.660 g, 81.34 mmol) and Cs₂CO₃(53.004 g, 162.68 mmol) in DMF (300 mL) was stirred at 100° C. for 16 hrs. The mixture was filtered and the filtrate was diluted with EtOAc (300 mL). The mixture was washed with brine, dried over anhydrous Na₂SO₄ and concentrated to give the title compound as a yellow solid (4.5 g, yield 24%). ¹H NMR (400 MHz, CDCl₃): δ 9.07 (s, 1H), 4.54˜4.46 (m, 1H), 3.98˜3.94 (m, 2H), 3.47˜3.40 (m, 2H), 2.03˜1.89 (m, 4H).

### Description D36

### 4-Nitro-1-(tetrahydro-2H-pyran-4-yl)-1H-pyrazol-3-ol (D36)

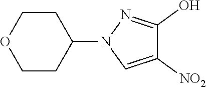 [SMILES: O=[N+]([O-])c1cn(C2CCOCC2)nc1O]

A mixture of D35 (1.0 g, 4.32 mmol) and KOH (2.0 g) in H₂O (30 mL) was stirred at 100° C. for 2 days. The mixture was washed with EtOAc (2×15 mL). The aqueous phase neutralized with 6N HCl to pH=3 and extracted with EtOAc (2×50 mL). The combined organic layers were dried over anhydrous Na₂SO₄, filtered and concentrated to give the title compound as yellow solid (730 mg, yield 79%). LC-MS: 214.3 [M+H]⁺.

### Description D37

### tert-Butyl (3-((4-nitro-1-(tetrahydro-2H-pyran-4-yl)-1H-pyrazol-3-yl)oxy)propyl) carbamate (D37)

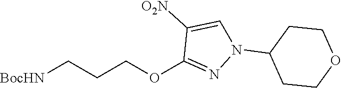 [SMILES: CC(C)(C)OC(=O)NCCCOc1nn(C2CCOCC2)cc1[N+](=O)[O-]]

A mixture of D36 (426 mg, 2.00 mmol), tert-butyl (3-bromopropyl)carbamate (476 mg, 2.00 mmol) and K₂CO₃ (553 mg, 4.00 mmol) in DMF (20 mL) were stirred at 60° C. for 3 hrs. The mixture was poured into ice water (100 mL) and extracted with EtOAc (2×60 mL). The combined organic layer was washed with brine, dried over anhydrous Na₂SO₄ and concentrated. The crude was purified by column chromatography on silica gel (PE:EtOAc=2:1) to give the title compound as a yellow solid (623 mg, yield 84%).

### Description D38

### tert-Butyl (3-((4-amino-1-(tetrahydro-2H-pyran-4-yl)-1H-pyrazol-3-yl)oxy)propyl) carbamate (D38)

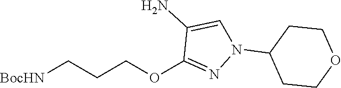 [SMILES: CC(C)(C)OC(=O)NCCCOc1nn(C2CCOCC2)cc1N]

A mixture of D37 (623 mg, 1.68 mmol), Fe (300 mg) and NH₄Cl (300 mg) in EtOH/H₂O (20 mL/5 mL) was stirred at 100° C. for 2 hrs. The mixture was filtered and concentrated. The residue was diluted with EtOAc (60 mL). The mixture was washed with brine, dried over and concentrated to give the title compound as black oil (256 mg, yield 45%).

### Description D39

### tert-Butyl N-{3-[(4-{[(tert-butoxy)carbonyl]amino}-1-(oxan-4-yl)-1H-pyrazol-3-yl)oxy]propyl}carbamate (D39)

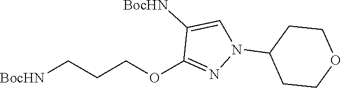 [SMILES: CC(C)(C)OC(=O)NCCCOc1nn(C2CCOCC2)cc1NC(=O)OC(C)(C)C]

To a solution of D38 (256 mg, 0.75 mmol) in THF (18 mL) was added LiHMDS (0.9 mL, 0.90 mmol) at 0° C. under nitrogen. The reaction was stirred at 0° C. for 0.5 hour. Then a solution of Boc₂O (197 mg, 0.90 mmol) in THF (2 mL) was added dropwise. The reaction was stirred at 0° C. for 0.5 hour and then at room temperature for 16 hrs. The mixture was poured into aq.NH₄Cl (60 mL) and extracted with EtOAc (2×50 mL). The combined organic layer was washed with brine, dried and concentrated. The crude was purified by chromatography on silica gel (PE:EtOAc=1:1) to give the title compound as a white solid (204 mg, yield 62%). LC-MS: 463.3 [M+Na]⁺.

### Description D40

### tert-Butyl N-{3-[(4-{[(tert-butoxy)carbonyl]amino}-5-chloro-1-(oxan-4-yl)-1H-pyrazol-3-yl)oxy]propyl}carbamate (D40)

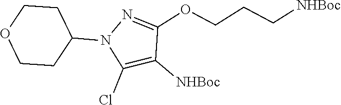 [SMILES: CC(C)(C)OC(=O)NCCCOc1nn(C2CCOCC2)c(Cl)c1NC(=O)OC(C)(C)C]

To a solution of D39 (164 mg, 0.37 mmol) in THF (20 mL) was added LDA (2 M in THF, 1.85 mL, 3.70 mmol) at −50° C. under nitrogen. The reaction was stirred at −20° C. for 0.5 h. Then a solution of 02016 (438 mg, 1.85 mmol) in THF (4 mL) was added dropwise. The reaction was stirred at −20° C. for 0.5 hour. The mixture was poured into aq. NH₄Cl (60 mL) and extracted with EtOAc (2×50 mL). The combined organic layer was washed with brine, dried and concentrated in vacuo. The crude was purified by column chromatography on silica gel (PE:EtOAc=2:1) to give the title compound as a white solid (83 mg, yield 62%).

### Description D41

### N-(3-((4-Amino-5-chloro-1-(tetrahydro-2H-pyran-4-yl)-1H-pyrazol-3-yl)oxy)propyl)-2,5-dichloropyrimidin-4-amine (D41)

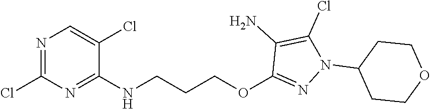 [SMILES: Nc1c(OCCCNc2nc(Cl)ncc2Cl)nn(C2CCOCC2)c1Cl]

A solution of D40 (83 mg, 0.17 mmol) in 4M HCl/dioxane (15 mL) was stirred at room temperature for 2 hrs. The reaction was concentrated and the residue was diluted with i-PrOH (19 mL). Et₃N (0.5 mL) and 2,4,5-trichloropyrimidine (32 mg, 0.17 mmol) were added and the resulting mixture was stirred at room temperature for 1 hour. The mixture was poured into H₂O (50 mL) and extracted with EtOAc (2×30 mL). The combined organic layers were washed with brine, dried and concentrated. The crude was purified by column chromatography on silica gel (PE:EtOAc=2:1) to give the title compound as a white solid (60 mg, yield 81%). LC-MS: 421.0 [M+H]⁺.

### Description D42

### 3-((2,5-Dichloropyrimidin-4-yl)amino)-2-fluoropropan-1˜01 (D42)

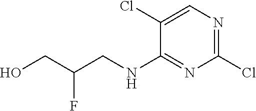 [SMILES: OCC(F)CNc1nc(Cl)ncc1Cl]

To a solution of 2,4,5-trichloropyrimidine (887 mg, 4.83 mmol) and 3-amino-2-fluoropropan-1-ol (450 mg, 4.83 mmol) in i-PrOH (10 mL) was added DIPEA (1.013 mL, 5.80 mmol) at 0° C. After stirred at 0° C. for 10 min, the mixture was quenched with water (10 mL), extracted with DCM (3×20 mL), dried over anhydrous Na₂SO₄ and concentrated to give the title compound to (570 mg, yield 49.1%). LC-MS: 240.0 [M+H]⁺.

### Description D43

### 3-((2,5-Dichloropyrimidin-4-yl)amino)-2-fluoropropyl methanesulfonate (D43)

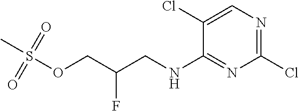 [SMILES: CS(=O)(=O)OCC(F)CNc1nc(Cl)ncc1Cl]

To a solution of D42 (270 mg, 1.125 mmol) in DCM (2 mL) was added MsCl (0.105 mL, 1.350 mmol) and DIPEA (0.295 mL, 1.687 mmol) at 25° C. The reaction was stirred at 25° C. for 1 hour. The mixture was quenched with water (4 mL), extracted with DCM (2×5 mL), dried over anhydrous Na₂SO₄ and concentrated to give the title compound (358 mg, yield 100%). LC-MS: 318.0 [M+H]⁺.

### Description D44

### 2,5-Dichloro-N-(2-fluoro-3-((5-methyl-4-nitro-1-(tetrahydro-2H-pyran-4-yl)-1H-pyrazol-3-yl)oxy)propyl)pyrimidin-4-amine (D44)

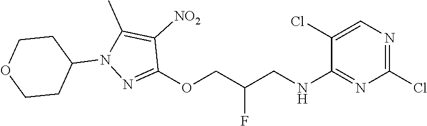 [SMILES: Cc1c([N+](=O)[O-])c(OCC(F)CNc2nc(Cl)ncc2Cl)nn1C1CCOCC1]

To a solution of D5 (275 mg, 1.210 mmol) in DMF (5 mL) was added K₂CO₃ (152 mg, 1.100 mmol) and D43 (350 mg, 1.100 mmol) at 25° C. The mixture was stirred at 60° C. for 2 hrs. The mixture was quenched with water (10 mL), extracted with DCM (3×20 mL), dried over anhydrous Na₂SO₄ and concentrated. The crude was purified by column chromatography on silica gel (PE:EtOAc=5:1 to 1:1) to give the title compound (50 mg, 0.111 mmol, yield 10.1%). LC-MS: 449.1 [M+H]⁺.

### Description D45

### 3-((2,5-Dichloropyrimidin-4-yl)amino)pentan-1-ol (D45)

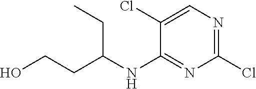

To a solution of 3-aminopentan-1-ol (500 mg, 4.85 mmol) in i-PrOH (6 mL) at 0° C. were added Et₃N (1.23 g, 12.1 mmol) and 2,4,5-trichloropyrimidine (930 mg, 5.07 mmol) dropwise. The reaction was stirred at 0° C. for 2 hrs. The mixture was filtered and washed with Et₂O (10 mL). The filtrate was washed with H₂O (2×8 mL) dried over anhydrous Na₂SO₄ and concentrated. The crude was purified by flash column chromatography on silica gel (PE:EtOAc=10:1 to 3:1) to give the title compound as an off-white solid (900 mg, yield 74%). LC-MS: 250.0 [M+H]⁺.

### Description D46

### 3-((2,5-Dichloropyrimidin-4-yl)amino)pentyl methanesulfonate (D46)

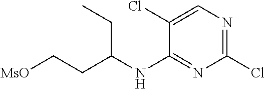 [SMILES: CCC(CCOS(C)(=O)=O)Nc1nc(Cl)ncc1Cl]

To a solution of D45 (0.40 g, 1.60 mmol) in CH₂Cl₂ (10 mL) were added Et₃N (486 mg, 4.8 mmol) and MsCl (366 mg, 3.2 mmol) at 0° C. The reaction was stirred at 25° C. for 2 hrs. The mixture was poured into saturated NH₄Cl aqueous solution (10 mL) and extracted with CH₂Cl₂ (2×10 mL). The organic layers were washed with brine, dried over anhydrous Na₂SO₄ and concentrated. Then purified by flash column chromatography on silica gel (PE:EtOAc=10:1 to 4:1) to give the title compound as yellow oil (0.5 g, yield 95%). LC-MS: 328.2 [M+H]⁺.

### Description D47

### 2,5-Dichloro-N-(14(5-methyl-4-nitro-1-(tetrahydro-2H-pyran-4-yl)-1H-pyrazol-3-yl)oxy)pentan-3-yl)pyrimidin-4-amine (D47)

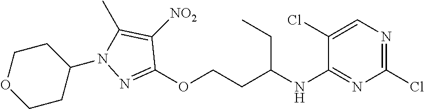 [SMILES: CCC(CCOc1nn(C2CCOCC2)c(C)c1[N+](=O)[O-])Nc1nc(Cl)ncc1Cl]

To a solution of D46 (433 mg, 1.32 mmol) in DMF (4 mL) were added K₂CO₃ (304 mg, 2.2 mmol) and D5 (200 mg, 0.880 mmol). The reaction was stirred at 80° C. for 30 min. The cooled mixture was poured into saturated NH₄Cl aqueous solution (15 mL) and extracted with Et₂O (2×10 mL). The organic layers were washed with brine, dried over anhydrous Na₂SO₄ and concentrated. The crude was purified by flash column chromatography on silica gel (PE:EtOAc=8:1 to 3:1) to give the title compound as an off-white solid (230 mg, yield 56%). LC-MS: 459.3 [M+H]⁺.

### Description D48

### 4-(3-Bromo-5-methyl-4-nitro-1H-pyrazol-1-yl)tetrahydro-2H-pyran-3-ol (D48)

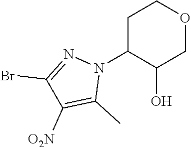 [SMILES: Cc1c([N+](=O)[O-])c(Br)nn1C1CCOCC1O]

To a solution of 3-bromo-5-methyl-4-nitro-1H-pyrazole (4 g, 19.42 mmol) in DMF (100 mL) were added K₂CO₃ (8.05 g, 58.25 mmol) and 3,7-dioxabicyclo[4.1.0]heptane (5.83 g, 58.26 mmol) at room temperature. The reaction was stirred at 80° C. for 10 hrs. The mixture was poured into water (200 mL) and extracted with EtOAc (3×200 mL). The combined organic layers were washed with brine, dried over anhydrous Na₂SO₄ and concentrated. The crude was purified by flash column chromatography (PE:EtOAc=1:1) to give the title compound as yellow oil (2 g, yield 33%).

### Description D49

### 3-Bromo-1-(3-fluorotetrahydro-2H-pyran-4-yl)-5-methyl-4-nitro-1H-pyrazole (D49)

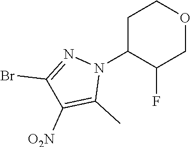 [SMILES: Cc1c([N+](=O)[O-])c(Br)nn1C1CCOCC1F]

To a solution of D48 (2 g, 6.53 mmol) in CH₂Cl₂ (50 mL) was added DAST (2.11 g). The reaction was stirred at 25° C. for 1 hour. The mixture was poured into NaHCO₃ (sat., 100 mL) and extracted with CH₂Cl₂ (3×200 mL). The organic layer was concentrated and the crude was purified by column chromatography on silica gel (PE:EtOAc=1:1) to give the title compound as yellow oil (1 g, yield 50%).

### Description D50

### 1-(3-Fluorotetrahydro-2H-pyran-4-yl)-5-methyl-4-nitro-1H-pyrazol-3-ol (D50)

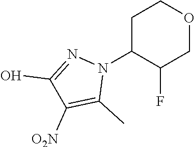 [SMILES: Cc1c([N+](=O)[O-])c(O)nn1C1CCOCC1F]

To a solution of D49 (1 g, 3.25 mmol) in H₂O (30 mL) were added KOH (910.5 mg, 16.23 mmol). The reaction was stirred at 120° C. for 10 hrs. The mixture was washed with EtOAc (50 mL) and the aqueous phase was neutralized with 2N HCl to pH=3˜4. The mixture was extracted with EtOAc (2×200 mL). The combined organic layers was concentrated to give the title compound as a yellow solid (400 mg, yield 50%). LC-MS: 246.3 [M+H]⁺.

### Description D51

### tert-Butyl (4-((1-(3-fluorotetrahydro-2H-pyran-4-yl)-5-methyl-4-nitro-1H-pyrazol-3-yl) oxy)butan-2-yl)carbamate (D51)

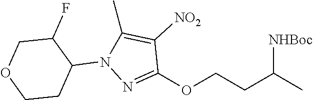 [SMILES: Cc1c([N+](=O)[O-])c(OCCC(C)NC(=O)OC(C)(C)C)nn1C1CCOCC1F]

To a solution of D50 (400 mg, 1.63 mmol) in DMF (10 mL) were added K₂CO₃ (689 mg, 4.9 mmol) and 3-((tert-butoxycarbonyl)amino)butyl methanesulfonate (654 mg, 2.45 mmol). The reaction was stirred at 80° C. for 2 hrs. The mixture was poured into water (100 mL) and extracted with EtOAc (3×100 mL). The combined organic layer was washed with brine, dried over anhydrous Na₂SO₄ and concentrated. The crude was purified by flash column chromatography on silica gel (PE:EtOAc=1:1) to give the title compound as yellow solid (400 mg, yield 65%). LC-MS: 439.2 [M+Na]⁺.

### Description D52

### 4-((1-(3-Fluorotetrahydro-2H-pyran-4-yl)-5-methyl-4-nitro-1H-pyrazol-3-yl)oxy) butan-2-amine, TFA salt (D52)

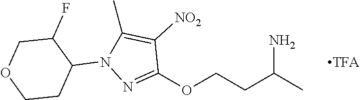 [SMILES: Cc1c([N+](=O)[O-])c(OCCC(C)N)nn1C1CCOCC1F]

To a solution of D51 (400 mg) in CH₂Cl₂ (20 mL) was added TFA (3 mL). The reaction was stirred at room temperature for 1 hour. The mixture was concentrated to give the title compound as yellow oil (250 mg, yield 83%). LC-MS: 317.3 [M+H]⁺.

### Description D53

### 2,5-Dichloro-N-(4-((1-(3-fluorotetrahydro-2H-pyran-4-yl)-5-methyl-4-nitro-1H-pyrazol-3-yl)oxy)butan-2-yl)pyrimidin-4-amine (D53)

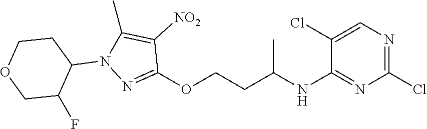 [SMILES: Cc1c([N+](=O)[O-])c(OCCC(C)Nc2nc(Cl)ncc2Cl)nn1C1CCOCC1F]

To a solution of D52 (250 mg, 0.79 mmol) in i-PrOH (10 mL) were added Et₃N (240 mg, 2.4 mmol) and 2,4,5-trichloropyrimidine (145 mg, 0.79 mmol). The reaction was stirred at 30° C. for 1 hour. The mixture was poured into water (50 mL) and extracted with EtOAc (100 mL). The organic layer was washed with brine, dried over anhydrous Na₂SO₄ and concentrated to give the title compound as a yellow solid (330 mg, yield 89%). LC-MS: 463.2 [M+H]⁺.

### Description D54

### tert-Butyl (4-((5-methyl-4-nitro-1-(tetrahydro-2H-pyran-4-yl)-1H-pyrazol-3-yl)oxy)butan-2-yl)carbamate (D54)

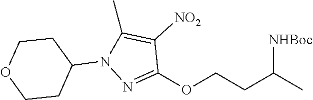 [SMILES: Cc1c([N+](=O)[O-])c(OCCC(C)NC(=O)OC(C)(C)C)nn1C1CCOCC1]

A solution of D5 (600 mg, 2.64 mmol), 3-((tert-butoxycarbonyl)amino)butyl methanesulfonate (847.5 mg, 3.17 mmol) and K₂CO₃ (729.7 mg, 5.28 mmol) in DMF (50 mL) was stirred at 100° C. for 2 hrs. The mixture was poured into ice water and extracted with EtOAc (3×100 mL). The combined organic layer was washed with brine, dried over anhydrous Na₂SO₄ and concentrated. The crude was purified by column chromatography on silica gel (PE:EtOAc=3:1) to give the title compound as an off-white solid (820 mg, yield 78%). LC-MS: 421.3 [M+Na]⁺.

### Description D55

### 4-((5-Methyl-4-nitro-1-(tetrahydro-2H-pyran-4-yl)-1H-pyrazol-3-yl)oxy)butan-2-Amine, TFA salt (D55)

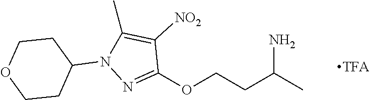 [SMILES: Cc1c([N+](=O)[O-])c(OCCC(C)N)nn1C1CCOCC1]

A solution of D54 (820 mg, 2.1 mmol) in CH₂Cl₂ (20 mL) was added TFA (5 mL). The mixture was stirred at room temperature for 2 hrs. The mixture was concentrated in vacuo to give the title compound as an off-white solid (800 mg, yield 98%). LC-MS: 299.4 [M+H]⁺.

### Description D56

### 5-Bromo-2-chloro-N-(4-((5-methyl-4-nitro-1-(tetrahydro-2H-pyran-4-yl)-1H-pyrazol-3-yl)oxy)butan-2-yl)pyrimidin-4-amine (D56)

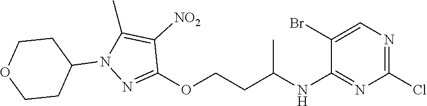 [SMILES: Cc1c([N+](=O)[O-])c(OCCC(C)Nc2nc(Cl)ncc2Br)nn1C1CCOCC1]

To a solution of D55 (300 mg, 0.76 mmol), Et₃N (153.8 mg, 1.52 mmol) in i-PrOH (30 mL) was added 5-bromo-2,4-dichloropyrimidine (346.4 mg, 1.52 mmol). The mixture was stirred overnight at room temperature. The mixture was filtered and the filter cake was dried to give the title compound as an off-white solid (280 mg, yield 75%). LC-MS: 491.1 [M+H]⁺.

### Description D57

### Ethyl 3-(benzylamino)-2-methylbutanoate (D57)

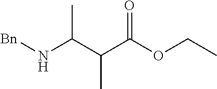 [SMILES: CCOC(=O)C(C)C(C)NCc1ccccc1]

To a solution of ethyl 2-methyl-3-oxobutanoate (15.0 g, 104.04 mmol) in MeOH (200 mL) was added BnNH₂ (16.7 g, 156.07 mmol) at 0° C. The reaction was stirred at 0° C. for 1 hour. Then sodium cyanoborohydride (13.1 g, 208.08 mmol) was added. The reaction was stirred overnight at room temperature. The mixture was concentrated and the crude purified by column chromatography on silica gel (PE:EtOAc=3:1) to give the title compound as a white solid (16.5 g, yield 67%).

### Description D58

### 3-(Benzylamino)-2-methylbutan-1-ol (D58)

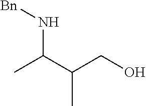

To a solution of D57 (16.5 g, 70.12 mmol) in THF (200 mL) was added LiAlH₄ (5.3 g, 140.23 mmol) at 0° C. The reaction was stirred at room temperature for 2 hrs and H₂O (11 mL) was added dropwise at 0° C. The mixture was dried over anhydrous Na₂SO₄, filtered and concentrated to give the title compound as clear oil (10.5 g, yield 77%).

### Description D59

### tert-Butyl (4-hydroxy-3-methylbutan-2-yl)carbamate (D59)

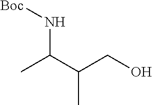

To a solution of D58 (2.0 g, 10.35 mmol) in MeOH (30 mL) was added Pd(OH)₂. The resulting mixture was stirred at 60° C. for 16 hrs under hydrogen. The reaction was cooled to 0° C., then Et₃N (3.0 mL, 20.70 mmol) and a solution of Boc₂O (2.2 g, 10.35 mmol) in MeOH (2 mL) was added dropwise. The reaction was stirred at room temperature for 2 hour. The mixture was filtered and the filtrate was concentrated. The crude was purified by column chromatography on silica gel (PE:EtOAc=2:1) to give the title compound as a white solid (1.5 g, yield 71%).

### Description D60

### 3-((tert-Butoxycarbonyl)amino)-2-methylbutyl methanesulfonate (D60)

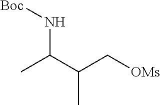 [SMILES: CC(COS(C)(=O)=O)C(C)NC(=O)OC(C)(C)C]

To a solution of D59 (1.5 g, 7.38 mmol) in CH₂Cl₂ (20 mL) was added Et₃N (2.3 g, 22.14 mmol) and MsCl (1.3 g, 11.07 mmol) at 0° C. The reaction was stirred at 0° C. for 0.5 hour. The mixture was poured into ice water (20 mL) and extracted with CH₂Cl₂ (2×20 mL). The organic layer was washed with aq. NaHCO₃ (2×30 mL), dried, filtered and concentrated to give the title compound as a white solid (2.3 g, crude).

### Description D61

### tert-Butyl (3-methyl-4-((5-methyl-4-nitro-1-(tetrahydro-2H-pyran-4-yl)-1H-pyrazol-3-yl)oxy)butan-2-yl)carbamate (D61)

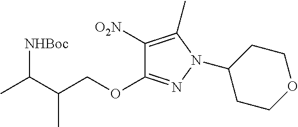 [SMILES: Cc1c([N+](=O)[O-])c(OCC(C)C(C)NC(=O)OC(C)(C)C)nn1C1CCOCC1]

A mixture of D60 (1.486 g, 5.28 mmol), D5 (400 mg, 1.76 mmol) and K₂CO₃ (730 mg, 5.28 mmol) in DMF (20 mL) was stirred at 80° C. for 4 hrs. The mixture was poured into ice water (60 mL) and extracted with EtOAc (2×30 mL). The combined organic layers were washed with brine (3×20 mL), dried and concentrated. The residue was purified by column chromatography on silica gel (PE:EtOAc=1:1) to give the title compound as yellow oil (240 mg, yield 33%).

### Description D62

### 3-Methyl-4-((5-methyl-4-nitro-1-(tetrahydro-2H-pyran-4-yl)-1H-pyrazol-3-yl)oxy) butan-2-amine, HCl salt (D62)

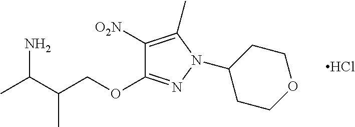 [SMILES: Cc1c([N+](=O)[O-])c(OCC(C)C(C)N)nn1C1CCOCC1 ; Cl]

A mixture of D61 (240 mg, 0.58 mmol) in HCl/MeOH (3M in MeOH, 6 mL) was stirred at room temperature for 1 hour. The mixture was concentrated in vacuo to give the title compound as a yellow solid (182 mg, crude).

### Description D63

### 2,5-Dichloro-N-(3-methyl-4-((5-methyl-4-nitro-1-(tetrahydro-2H-pyran-4-yl)-1H-pyrazol-3-yl)oxy)butan-2-yl)pyrimidin-4-amine (D63)

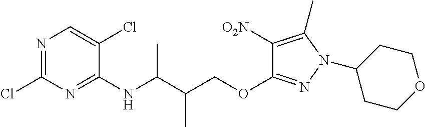 [SMILES: Cc1c([N+](=O)[O-])c(OCC(C)C(C)Nc2nc(Cl)ncc2Cl)nn1C1CCOCC1]

To a solution of D62 (182 mg, 0.58 mmol) in i-PrOH (15 mL) were added Et₃N (294 mg, 2.90 mmol) and 2,4,5-trichloropyrimidine (213 mg, 1.16 mmol). The reaction was stirred at room temperature for 1 hour. The reaction was poured into ice water (30 mL) and extracted with EtOAc (2×30 mL). The organic layers were dried, filtered and concentrated. The crude was purified by column chromatography on silica gel (PE:EtOAc=1:1) to give the title compound as a yellow solid (220 mg, yield 82%).

### Description D64

### 3-(Benzylamino)-4-methoxybutan-1-ol (D64)

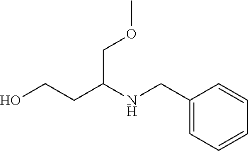

To a solution of methyl 3-(benzylamino)-4-methoxybutanoate (which could be prepared from WO2016014463A) (4.0 g, 16.9 mmol) in dry THF (60 mL) under argon at 0° C. was added LiAlH₄ (3.23 g, 85 mmol) portion wise. The reaction was stirred at 25° C. for 2 hrs. The reaction was quenched by addition of water (12 mL, diluted with 80 mL THF). The resultant was dried, filtered and concentrated. The crude was purified by column chromatography on silica gel (CH₂Cl₂:CH₃OH=50:1 to 10:1) to give the title compound as yellow oil (2.0 g, yield 51%). LC-MS: 210.1 [M+H]⁺.

### Description D65

### 3-Amino-4-methoxybutan-1-ol, TFA salt (D65)

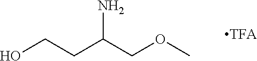

A mixture of D64 (2.0 g, 9.56 mmol), Pd(OH)₂ (1 g) and TFA (2 mL) in CH₃OH (200 mL) was stirred under hydrogen at 50° C. for 16 hrs. The mixture was filtered and concentrated to give the compound as yellow oil (1.3 g, crude). LC-MS: 120.1 [M+H]⁺.

### Description D66

### 3-((2,5-Dichloropyrimidin-4-yl)amino)-4-methoxybutan-1-ol (D66)

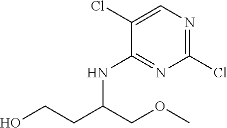 [SMILES: COCC(CCO)Nc1nc(Cl)ncc1Cl]

To a solution of D65 (977 mg, 8.19 mmol) in i-PrOH (20 mL) at 0° C. were added DIPEA (3.17 g, 24.6 mmol) and 2,4,5-trichloropyrimidine (1.66 g, 9.03 mmol) dropwise. The reaction was stirred at 25° C. for 15 hrs. The mixture was concentrated and the crude was purified by flash column chromatography on silica gel (PE:EtOAc=3:1) to give the title compound as a white solid (880 mg, yield 40%). LC-MS: 266.0 [M+H]⁺.

### Description D67

### 3-((2,5-Dichloropyrimidin-4-yl)amino)-4-methoxybutyl methanesulfonate (D67)

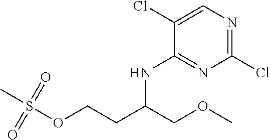 [SMILES: COCC(CCOS(C)(=O)=O)Nc1nc(Cl)ncc1Cl]

To a solution of D66 (0.88 g, 3.31 mmol) in CH₂Cl₂ (20 mL) at 0° C. were added Et₃N (1000 mg, 9.93 mmol) and MsCl (455 mg, 3.97 mmol). The reaction was stirred at 25° C. for 1 hour. The mixture was poured into water and extracted with CH₂Cl₂. The combined organic layers were washed with brine, dried over anhydrous Na₂SO₄ and concentrated to give the title compound as yellow oil (0.74 g, yield 65%). LC-MS: 344.0 [M+H]⁺.

### Description D68

### 2,5-Dichloro-N-(1-methoxy-4-((5-methyl-4-nitro-1-(tetrahydro-2H-pyran-4-yl)-1H-pyrazol-3-yl)oxy)butan-2-yl)pyrimidin-4-amine (D68)

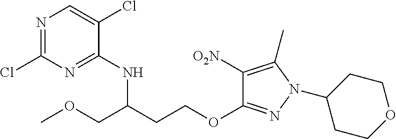 [SMILES: COCC(CCOc1nn(C2CCOCC2)c(C)c1[N+](=O)[O-])Nc1nc(Cl)ncc1Cl]

A mixture of D67 (742 mg, 2.15 mmol), K₂CO₃ (894 mg, 6.48 mmol) and D5 (440 mg, 1.95 mmol) in DMSO (20 mL) was stirred at 75° C. for 30 min. The mixture was poured into water and extracted with EtOAc. The combined organic layers were washed with brine, dried over anhydrous Na₂SO₄ and concentrated. The crude was purified by flash column chromatography on silica gel (PE:EtOAc=4:1 to 1:1) to give the title compound as an off-white solid (247 mg, yield 24%). LC-MS: 475.1 [M+H]⁺.

### Description D69

### 3-Methyl-4-nitro-1-(tetrahydro-2H-pyran-2-yl)-1H-pyrazole (D69)

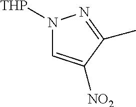

To a solution of 3-methyl-4-nitro-1H-pyrazole (50 g, 393 mmol) in EtOAc (500 mL) was added DHP (49.6 g, 590 mmol) and pTsOH.H₂O (3.66 g, 20 mmol) at room temperature. The reaction was stirred overnight at room temperature. Et₃N (6 mL) was added and the organic was washed with brine (2×300 mL). The organic layer was dried over Na₂SO₄ and concentrated in vacuo to give the title compound as colorless oil, which was used in next step without further purification (65 g, 78%). LC-MS: 233.9 [M+Na]⁺.

### Description D70

### 5-Chloro-3-methyl-4-nitro-1-(tetrahydro-2H-pyran-2-yl)-1H-pyrazole (D70)

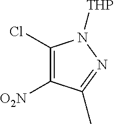

To a solution of D69 (65 g, 308 mmol) in dry THF (500 mL) was added LiHMDS (370 mL, 1 M in THF) at −70° C. under nitrogen. The reaction was stirred at −70° C. for 45 min and then a solution of 02016 (218 g, 924 mmol) in THF (400 mL) was added dropwise. The reaction was stirred at room temperature for 30 min. NH₄Cl solution (sat.) was added and the mixture was extracted with EtOAc (3×400 mL). The combined organic layers were washed with brine (500 mL), dried over Na₂SO₄ and concentrated in vacuo. The crude was purified by column chromatography on silica gel (PE:EtOAc=8:1 to 6:1) to give the title compound as a yellow solid (69 g, yield 91%). LC-MS: 267.8 [M+Na]⁺.

### Description D71

### tert-Butyl (4-((3-methyl-4-nitro-1-(tetrahydro-2H-pyran-2-yl)-1H-pyrazol-5-yl)oxy) butan-2-yl)carbamate (D71)

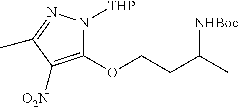

To a solution of tert-butyl (4-hydroxybutan-2-yl)carbamate (1 g, 5.3 mmol) in DMF (20 mL) was added NaH (0.53, 13.25 mmol) at 0° C. After stirred for 1 hour, D70 (1.04 g, 4.2 mmol) was added. The reaction was stirred at 0° C. for 6 hours and then diluted with EtOAc. The organic layer was washed with brine, dried over Na₂SO₄ and concentrated. The crude was purified by column chromatography on silica gel (PE:EtOAc=20:1 to 10:1) to give the title compound (1.2 g, yield 70%).

### Description D72

### (4-((3-Methyl-4-nitro-1H-pyrazol-5-yl)oxy)butan-2-amine, HCl salt (D72)

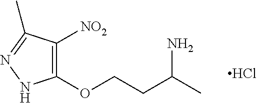

To a solution of D71 (1.19 g) in CH₃OH (20 mL) was added con. HCl (1.2 mL) at room temperature. The mixture was stirred at room temperature for 2 hrs. The organic solvent was removed in vacuum to give the crude which was used in next step without further purification (920 mg, crude).

### Description D73

### 2,5-Dichloro-N-(4-((3-methyl-4-nitro-1H-pyrazol-5-yl)oxy)butan-2-yl)pyrimidin-4-amine (D73)

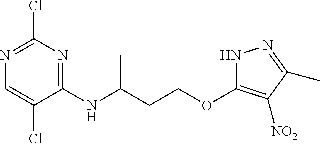 [SMILES: Cc1n[nH]c(OCCC(C)Nc2nc(Cl)ncc2Cl)c1[N+](=O)[O-]]

To a solution of D72 (1.92 g, crude) in i-PrOH (50 mL) was added 2,4,5-trichloropyrimidine (1.97 g, 10.7 mmol) and Et₃N (2.7 mL, 27 mmol) at room temperature. The reaction was stirred overnight at room temperature. The organic solvent was removed in vacuo. The crude was was purified by column chromatography on silica gel (PE:EtOAc=1:1) to give the title compound (1.1 g).

### Description D74

### Ethyl 2-(3-(3-((2,5-dichloropyrimidin-4-yl)amino)butoxy)-5-methyl-4-nitro-1H-pyrazol-1-yl)-2-methylpropanoate (D74)

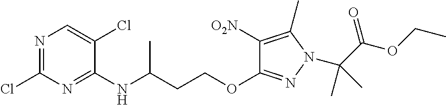 [SMILES: CCOC(=O)C(C)(C)n1nc(OCCC(C)Nc2nc(Cl)ncc2Cl)c([N+](=O)[O-])c1C]

To a solution of ethyl 2-bromo-2-methylpropanoate (975 mg, 5 mmol) in DMF (60 mL) were added D73 (361 mg, 1 mmol) and K₂CO₃ (690 mg, 5 mmol). Then reaction was heated to 80° C. and stirred overnight. The mixture was diluted with ice water and extracted with EtOAc (2×100 mL). The organic layer was washed with brine, dried and concentrated. The crude was purified by column chromatography on silica gel (PE:EA=2:1) to give the title compound as clear oil (980 mg, yield 68%). LC-MS: 475.2 [M+H]⁺.

### Description D75

### 3-(3-(3-((2,5-Dichloropyrimidin-4-yl)amino)propoxy)-5-methyl-4-nitro-1H-pyrazol-1-yl)cyclobutanone (D75)

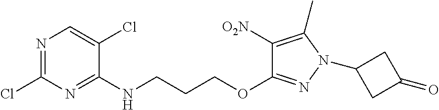 [SMILES: Cc1c([N+](=O)[O-])c(OCCCNc2nc(Cl)ncc2Cl)nn1C1CC(=O)C1]

To a solution of D33 (1.5 g, 4.32 mmol) in anhydrous DMF (20 mL) was added NaH (207 mg, 5.17 mmol) at 0° C. under argon. The reaction was stirred at room temperature for 0.5 hour. 3-bromocyclobutanone (1.3 g, 8.73 mmol) was added and the reaction was stirred at room temperature for 2.5 hrs. Water was added and the resultant was extracted with EtOAc (3×20 mL). The combined organics were washed with water (4×30 mL) and brine (20 mL), dried over anhydrous Na₂SO₄, filtered and concentrated. The crude was purified by column chromatography on silica gel (CH₂Cl₂:MeOH=100:1) to give the title compound as a white solid (690 mg, yield 38%). LC-MS: 415.3 [M+H]⁺.

### Description D76

### 2,5-Dichloro-N-(3-((5-methyl-1-(3-morpholinocyclobutyl)-4-nitro-1H-pyrazol-3-yl)oxy)propyl)pyrimidin-4-amine (D76)

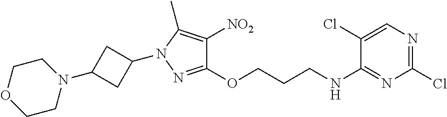 [SMILES: Cc1c([N+](=O)[O-])c(OCCCNc2nc(Cl)ncc2Cl)nn1C1CC(N2CCOCC2)C1]

To a mixture of D75 (100 mg, 0.241 mmol), morpholine (60 mg, 0.689 mmol), AcOH (20 mg, 0.33 mmol) and 4 Å molecular sieves (100 mg) in anhydrous CH₂Cl₂ (4 mL) was added NaBH₃CN (32 mg, 0.509 mmol) at 0° C. under argon. The reaction was stirred overnight under argon at room temperature. The mixture was filtered and the filtrate was treated with aqueous NaHCO₃. After aqueous workup and concentration, the residue was purified by prep-TLC to give the title compound as a white solid (100 mg, yield 85%). LC-MS: 486.2 [M+H]⁺.

### Description D77

### tert-Butyl ((2R)-4-((3-methyl-4-nitro-1-(tetrahydro-2H-pyran-2-yl)-1H-pyrazol-5-yl) oxy) butan-2-yl)carbamate (D77)

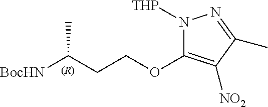

To a solution of NaH (96.8 g, 2.42 mol in DMF (500 mL) was added (R)-tert-butyl (4-hydroxybutan-2-yl)carbamate (249.8 g, 1.32 mol)) at 0° C. The mixture was stirred at 0° C. for 1 hour and D70 (260 g, 1.1 mol) was added and stirred at room temperature for 4 hr. The reaction mixture was poured into ice water and extracted with EtOAc (3×1 L). The combined organic layers were washed with brine, dried over Na₂SO₄ and concentrated. The residue was slurry in PE:EtOAc=3:1 to give the title compound as a yellow solid (350 g, crude).

### Description D78

(R)-4-((3-methyl-4-nitro-1H-pyrazol-5-yl)oxy)butan-2-amine, HCl salt (D78)

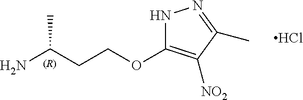

A solution of D77 (350 g crude, 0.88 mol) in 4N HCl (1 L) was stirred at room temperature for 4 hrs. The reaction mixture was concentrated in vacuo. The residue was slurry in EtOAc to give the target product as a yellow solid (250 g, yield 80%, two steps). LC-MS: 215.4 [M+H]⁺.

### Description D79

### (R)-2,5-Dichloro-N-(4-((3-methyl-4-nitro-1H-pyrazol-5-yl)oxy)butan-2-yl)pyrimidin-4-amine (D79)

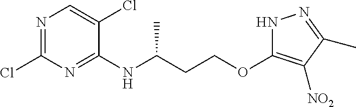 [SMILES: Cc1n[nH]c(OCC[C@@H](C)Nc2nc(Cl)ncc2Cl)c1[N+](=O)[O-]]

To a solution of D78 (250 g, 0.88 mol) and Et₃N (178.1 g, 1.76 mol) in MeOH (1 L) was added 2,4,5-trichloro pyrimidine (194.4 g, 1.06 mol). The mixture was stirred overnight at room temperature. The mixture was concentrated in vacuo, poured into the sat.NaCl (aq) and extracted with EtOAc (3×1 L). The combined organic layer was washed with brine, dried over Na₂SO₄ and concentrated in vacuo. The crude product was slurry in CH₂Cl₂/hexane=1:1 (1 L) and filtered to give the title compound as a yellow solid (180 g, yield 55%).

### Description D80

### 2,5-Dichloro-N—((R)-4-((5-methyl-4-nitro-1-((S)-tetrahydrofuran-3-yl)-1H-pyrazol-3-yl)oxy)butan-2-yl)pyrimidin-4-amine (D80)

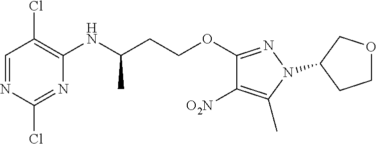 [SMILES: Cc1c([N+](=O)[O-])c(OCC[C@@H](C)Nc2nc(Cl)ncc2Cl)nn1[C@H]1CCOC1]

A solution of D79 (200 mg, 0.56 mmol), (R)-tetrahydrofuran-3-yl methanesulfonate (458 mg, 2.78 mmol) and Cs₂CO₃ (543 mg, 1.7 mmol) in DMA (15 mL) was heated to 80° C. and stirred for 16 hrs. The cooled mixture was poured into water (60 mL) and extracted with EtOAc (3×100 mL). The combined organic layers were washed with water, brine, dried over anhydrous Na₂SO₄ and concentrated in vacuo. The crude was purified by column chromatography on silica gel (PE:EtOAc=5:1 to 2:1) to give the title compound as a yellow oil (150 mg, yield 62.2%). LC-MS: 431.2 [M+H]⁺.

### Description D81

### 2,5-Dichloro-N—((R)-4-((5-methyl-4-nitro-1-((R)-tetrahydrofuran-3-yl)-1H-pyrazol-3-yl)oxy)butan-2-yl)pyrimidin-4-amine (D81)

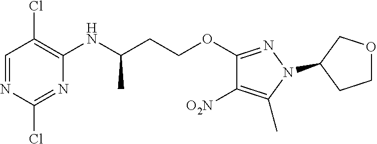 [SMILES: Cc1c([N+](=O)[O-])c(OCC[C@@H](C)Nc2nc(Cl)ncc2Cl)nn1[C@@H]1CCOC1]

A solution of D79 (200 mg, 0.56 mmol), (S)-tetrahydrofuran-3-yl methanesulfonate (276 mg, 1.67 mmol) and Cs₂CO₃ (543 mg, 1.7 mmol) in DMA (10 mL) was heated to 80° C. and stirred for 16 hrs. The cooled mixture was poured into water (60 mL) and extracted with EtOAc (3×50 mL). The combined organic layers were washed with water, brine, dried over anhydrous Na₂SO₄ and concentrated in vacuo. The crude was purified by column chromatography on silica gel (PE:EtOAc=5:1 to 2:1) to give the title compound as yellow oil (130 mg, yield 54%). LC-MS: 431.2 [M+H]⁺.

### Description D82

### 1,4-Dioxaspiro[4.5]decan-8-yl methanesulfonate (D82)

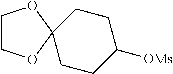 [SMILES: CS(=O)(=O)OC1CCC2(CC1)OCCO2]

To a solution of 1,4-dioxaspiro[4.5]decan-8-ol (12 g, 76 mmol), TEA (11.5 g, 114 mmol) in DCM (110 mL) at 0˜5° C. was added MsCl (11.3 g, 98.7 mmol) dropwise. Then the solution was stirred for 1 hour at room temperature. Water (50 mL) was added, the combined organic layer was washed with satur. NaHCO₃, brine, dried over Na₂SO₄ and concentrated to give the title compound as a little yellow solid (18.5 g, yield 100%).

### Description D83

### 2,5-Dichloro-N-(3-((5-methyl-4-nitro-1-(1,4-dioxaspiro[4.5]decan-8-yl)-1H-pyrazol-3-yl)oxy)propyl)pyrimidin-4-amine (D83)

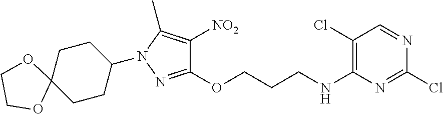 [SMILES: Cc1c([N+](=O)[O-])c(OCCCNc2nc(Cl)ncc2Cl)nn1C1CCC2(CC1)OCCO2]

A mixture of D33 (1.5 g, 4.32 mmol), D82 (2.05 g, 8.68 mmol) and K₂CO₃ (1.79 g, 12.95 mmol) in DMSO (20 mL) was stirred at 100° C. for 16 hrs. Water (30 mL) was added and the resultant was extracted with EtOAc (3×20 mL). The combined organics were washed with brine (50 mL), dried over anhydrous Na₂SO₄, filtered and concentrated. The crude was purified by column chromatography on silica gel (PE:EtOAc=10:1 to 3:1) to give the title compound as a white solid (1.4 g, yield 67%). LC-MS: 487.1 [M+H]⁺.

### Description D84

### 14-Chloro-5-{1,4-dioxaspiro[4.5]decan-8-yl}-4-methyl-8-oxa-2,5,6,12,16,17-hexaaza tricyclo[11.3.1.0^{3,7}]heptadeca-1(16),3,6,13(17),14-pentaene (D84)

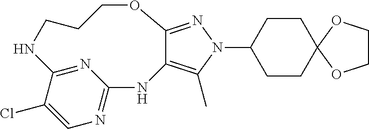 [SMILES: Cc1c2c(nn1C1CCC3(CC1)OCCO3)OCCCNc1nc(ncc1Cl)N2]

A mixture of D83 (1.4 g, 2.87 mmol), Fe (805 mg, 14.4 mmol) and NH₄Cl (1.53 g, 28.6 mmol) in EtOH (50 mL) and H₂O (10 mL) was stirred at 100° C. for 16 hrs. The reaction mixture was filtered and the filtrate was concentrated. The residue was re-dissolved in CH₂Cl₂ (100 mL) and then dried over anhydrous Na₂SO₄, filtered and concentrated to give the title compound as a white solid (500 mg, yield 42%). LC-MS: 421.3 [M+H]⁺.

### Description D85

### 4-{14-Chloro-4-methyl-8-oxa-2,5,6,12,16,17-hexaazatricyclo[11.3.1.0^{3,7}]heptadeca-1(16),3,6,13(17),14-pentaen-5-yl}cyclohexan-1-one (D85)

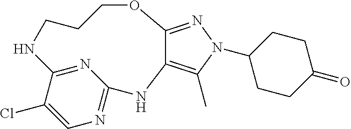 [SMILES: Cc1c2c(nn1C1CCC(=O)CC1)OCCCNc1nc(ncc1Cl)N2]

To a solution of D84 (750 mg, 1.78 mmol) in CH₂Cl₂ (20 mL) was added TFA (678 mg, 5.95 mmol). The reaction was stirred at 80° C. for 5 hrs. The mixture was concentrated and the residue was re-dissolved in CH₂Cl₂ (100 mL). Aq. NaHCO₃ was added until pH=8 and the mixture was extracted with CH₂Cl₂ (3×50 mL). The combined organics were washed with brine (100 mL), dried over anhydrous Na₂SO₄ and concentrated to give the title compound as a white solid (600 mg, yield 90%). LC-MS: 377.3 [M+H]⁺.

### Description D86

### Ethyl 4-fluoro-3-oxobutanoate (D86)

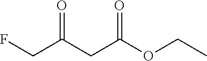

To a solution of EtOAc (2.74 g, 31.1 mmol) in dry THF (30 mL) under argon at −78° C. was added LDA (17 mL, 34 mmol) dropwise. The reaction was stirred at −78° C. for 1 hour. Ethyl 2-fluoroacetate (3.0 g, 28.3 mmol) was added dropwise at −78° C. and the mixture was allowed to warm to room temperature and stirred overnight under argon. The mixture was quenched with aq. HCl (1 M) and extracted with Et₂O. The organic layer was washed with brine (50 mL), dried over anhydrous Na₂SO₄, filtered and concentrated. The crude was purified by column chromatography on silica gel (PE:EtOAc=10:1˜5:1) to give the title compound as yellow oil (4.2 g, quant.). ¹H NMR (400 MHz, CDCl₃): δ 4.91 (d, J=47.6 Hz, 2H), 4.23 (q, J=7.2 Hz, 2H), 3.60 (d, J=3.6 Hz, 2H), 1.29 (t, J=7.2 Hz, 3H).

### Description D87

### Ethyl 4-fluoro-3-((4-methoxybenzyl)amino)butanoate (D87)

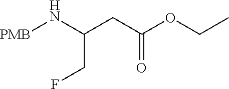

To a solution of D86 (1.0 g, 6.76 mmol) and HOAc (609 mg, 10.14 mmol) in EtOH (10 mL) at 0° C. was added (4-methoxyphenyl)methanamine (1.39 g, 10.1 mmol) dropwise. The mixture was stirred at room temperature for 2.5 hrs. After cooled to 0° C., the mixture was added HOAc (1.42 g, 23.65 mmol) and NaBH₃CN (1.34 g, 20.3 mmol). The reaction was stirred at room temperature for another 3 hrs. The reaction was quenched with Na₂CO₃ and extracted with EtOAc (2×20 mL). The combined organic layer was dried over Na₂SO₄, filtered and concentrated. The crude was purified by chromatography on silica gel (CH₂Cl₂:CH₃OH=200:1 to 80:1) to give the title compound as yellow oil (1.4 g, yield 77%). ¹H NMR (400 MHz, CDCl₃): δ 7.24 (d, J=8.4 Hz, 2H), 6.86 (d, J=8.8 Hz, 2H), 4.53˜4.48 (m, 1H), 4.42˜4.35 (m, 1H), 4.14 (q, J=7.1 Hz, 2H), 3.79 (s, 3H), 3.78 (s, 2H), 3.33˜3.24 (m, 1H), 2.54˜2.52 (m, 2H), 1.26 (t, J=7.0 Hz, 3H).

### Description D88

### 4-Fluoro-3-((4-methoxybenzyl)amino)butan-1-ol (D88)

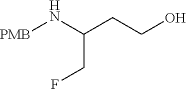

To a suspension of LiAlH₄ (118 mg, 3.11 mmol) in dry THF (5 mL) under argon at −60° C. was added a solution of D87 (0.70 g, 2.60 mmol) in dry THF (5 mL) dropwise. The reaction was stirred at −60° C. for 3 hrs. The mixture was quenched with water/THF (0.5 mL/5 mL) slowly at −60° C. After stirring for 30 mins, the mixture was filtered through Celite. The filtrate was concentrated to give the title compound as yellow oil (410 mg, yield 69%). ¹H NMR (400 MHz, CDCl₃): δ 7.24 (d, J=8.4 Hz, 2H), 6.87 (d, J=8.4 Hz, 2H), 4.63˜4.49 (m, 1H), 4.46˜4.32 (m, 1H), 3.88˜3.73 (m, 7H), 3.13˜3.01 (m, 1H), 1.76˜1.69 (m, 2H).

### Description D89

### 3-Amino-4-fluorobutan-1-ol (D89)

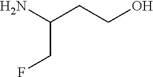

To a solution of D88 (2.0 g, 8.80 mmol) and HOAc (1 mL) in MeOH (50 mL) was added Pd(OH)₂/C (300 mg). The reaction was stirred under hydrogen at 35° C. for 48 hrs. The mixture was filtered through Celite and the filtrate was concentrated to give the title compound as yellow oil (1.0 g, crude).

### Description D90

### 3-((2,5-Dichloropyrimidin-4-yl)amino)-4-fluorobutan-1-ol (D90)

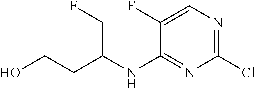 [SMILES: OCCC(CF)Nc1nc(Cl)ncc1F]

To a solution of D89 (650 mg, ca. 6.07 mmol) in i-PrOH (10 mL) was added Et₃N (1.84 g, 18.2 mmol) and 2,4,5-trichloropyrimidine (1.23 g, 6.68 mmol) at 0° C. 0° C. The reaction was quenched with aq. NH₄Cl (18 mL) and extracted with EtOAc (2×18 mL). The organic layer was washed with brine (20 mL), dried over Na₂SO₄, filtered and concentrated. The crude was purified by chromatography on silica gel (PE:EtOAc=10:1˜3:1) to give the title compound as a white solid (650 mg, yield 42%). LC-MS: 254.0 [M+H]⁺.

### Description D91

### 2,5-Dichloro-N-(1-fluoro-4-((5-methyl-4-nitro-1-(tetrahydro-2H-pyran-4-yl)-1H-pyrazol-3-yl)oxy)butan-2-yl)pyrimidin-4-amine (D91)

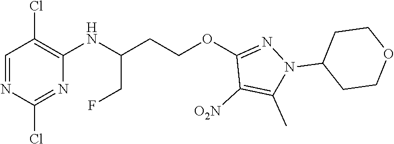 [SMILES: Cc1c([N+](=O)[O-])c(OCCC(CF)Nc2nc(Cl)ncc2Cl)nn1C1CCOCC1]

To a solution of D90 (300 mg, 1.18 mmol), D5 (282 mg, 1.24 mmol) and PPh₃ (619 mg, 2.36 mmol) in dry THF (12 mL) under argon at 0° C. was added DEAD (411 mg, 2.36 mmol) dropwise. The reaction was stirred overnight under argon at 26° C. The reaction was diluted with aq. HCl (0.5 M, 30 mL) and extracted with EtOAc (2×20 mL). The combined organics were washed with brine (30 mL), dried over anhydrous Na₂SO₄, filtered and concentrated. The residue was suspended in methanol (12 mL), stirred at room temperature for 30 min, filtered and the filter cake was collected, dried to give the title compound as a white solid (480 mg, yield 87.8%). ¹H NMR (400 MHz, CDCl₃): δ 8.03 (s, 1H), 5.85 (d, J=8.0 Hz, 1H), 4.76˜4.69 (m, 2H), 4.60 (dd, J=15.6, 3.6 Hz, 1H), 4.46 (t, J=5.6 Hz, 2H), 4.21˜4.17 (m, 1H), 4.13˜4.09 (m, 2H), 3.50 (t, J=12.0 Hz, 2H), 2.63 (s, 3H), 2.32˜2.18 (m, 4H), 1.75˜1.70 (m, 2H).

### Description D92

### 2,5-Dichloro-N-(4-((5-methyl-4-nitro-1-(1,4-dioxaspiro[4.5]decan-8-yl)-1H-pyrazol-3-yl)oxy)butan-2-yl)pyrimidin-4-amine (D92)

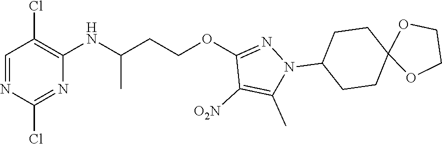 [SMILES: Cc1c([N+](=O)[O-])c(OCCC(C)Nc2nc(Cl)ncc2Cl)nn1C1CCC2(CC1)OCCO2]

A solution of D73 (4.5 g, 12.5 mmol), D82 (14.7 g, 62.5 mmol), NaI (1.8 g, 12.0 mmol) and K₂CO₃ (5.2 g, 37.5 mmol) in DMA (50 mL) was heated to 80° C. and stirred for 16 hrs. The mixture was poured into water (200 mL) and extracted with EtOAc (3×150 mL). The combined organic layers were washed with brine, dried over anhydrous Na₂SO₄ and concentrated in vacuo. The crude was purified by flash column chromatography on silica gel (PE:EtOAc=5:1 to 2:1) to give the title compound as a little yellow oil (2.5 g, yield 40%). LC-MS: 501.1 [M+H]⁺.

### Description D93

### 14-Chloro-5-{1,4-dioxaspiro[4.5]decan-8-yl}-4,11-dimethyl-8-oxa-2,5,6,12,16,17-hexaazatricyclo[11.3.1.0^{3,7}]heptadeca-1(16),3,6,13(17),14-pentaene (D93)

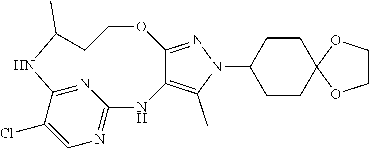 [SMILES: Cc1c2c(nn1C1CCC3(CC1)OCCO3)OCCC(C)Nc1nc(ncc1Cl)N2]

A solution of D92 (2.6 g, 5.2 mmol), Fe (2.6 g, 46.4 mmol) and NH₄Cl (5.2 g, 97.2 mmol) in EtOH and H₂O (50 mL, 3:1) was heated to 90° C. for 16 hrs. Sat. NaHCO₃(5 mL) was added and the mixture was stirred for 10 min, filtered, washed with DCM and concentrated. DCM (25 mL) was added and the mixture was stirred for 30 min, filtered and concentrated to give the title compound as a yellow solid (2.0 g, yield 89%). LC-MS: 435.3 [M+H]⁺.

### Description D94

### 4-{14-Chloro-4,11-dimethyl-8-oxa-2,5,6,12,16,17-hexaazatricyclo[11.3.1.0^{3,7}]heptadeca-1(16),3,6,13(17),14-pentaen-5-yl}cyclohexan-1-one (D94)

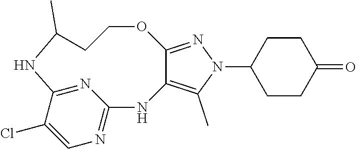 [SMILES: Cc1c2c(nn1C1CCC(=O)CC1)OCCC(C)Nc1nc(ncc1Cl)N2]

To a solution of D93 (2.5 g, 5.76 mmol) in DCM (50 mL) was added TFA (10 mL) and the reaction was stirred for 5 hrs at 60° C. The mixture was then cooled to room temperature and water (100 mL) was added. Sat. NaHCO₃ was added until pH>7 and the organic layer was washed with brine, dried over Na₂SO₄, filtered and concentrated. The crude was purified by column chromatography on silica gel (PE:EA=5:1˜1:1) to give the title compound (1.3 g, yield 58%). LC-MS: 391.2 [M+H]⁺.

### Description D95

### Trans-tert-butyl 4-(3-(3-((2,5-dichloropyrimidin-4-yl)amino)propoxy)-5-methyl-4-nitro-1H-pyrazol-1-yl)-3-fluoropiperidine-1-carboxylate (D95)

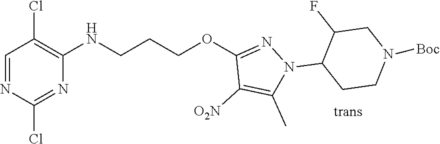 [SMILES: Cc1c([N+](=O)[O-])c(OCCCNc2nc(Cl)ncc2Cl)nn1C1CCN(C(=O)OC(C)(C)C)CC1F]

To a solution of cis-tert-butyl 3-fluoro-4-((methylsulfonyl)oxy)piperidine-1-carboxylate (858 mg, 2.88 mmol) in DMSO (6.0 mL) was added D33 (500 mg, 1.44 mmol) and K₂CO₃ (598 mg, 4.33 mmol). The reaction was stirred at 85° C. for 5 days. The mixture was diluted with water and extracted with EtOAc for three times. The combined organics were washed with brine, dried, filtered and concentrated. The crude was purified by column chromatography on silica gel (PE:EtOAc=1:1, followed by CH₂Cl₂:MeOH=20:1) to give the title compound as a yellow solid (385 mg, yield 48%). LC-MS: 548.3 [M+H]⁺.

### Description D96

Trans-tert-butyl 4-{14-chloro-4-methyl-8-oxa-2,5,6,12,16,17-hexaazatricyclo [11.3.1.0^{3,7}]heptadeca-1(16),3,6,13(17),14-pentaen-5-yl}-3-fluoropiperidine-1-carboxylate (D96)

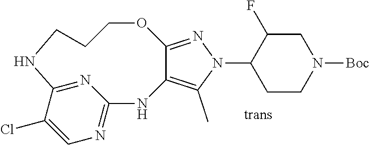 [SMILES: Cc1c2c(nn1C1CCN(C(=O)OC(C)(C)C)CC1F)OCCCNc1nc(ncc1Cl)N2]

To a mixture of D95 (385 mg, 0.702 mmol) and Fe (391 mg, 7.00 mmol) in EtOH/H₂O (10.0 mL/2.0 mL) was added NH₄Cl (374 mg, 7.00 mmol). The reaction was stirred at 100° C. overnight. The mixture was diluted with aq. NaHCO₃ and filtered. The filtrate was extracted with EtOAc for 3 times. The combined organics were dried, filtered and concentrated. The crude was purified by column chromatography on silica gel (CH₂Cl₂:MeOH=10:1) to give the title compound as yellow oil (125 mg, yield 36%). LC-MS: 482.4 [M+H]⁺.

### Description D97

Trans-14-chloro-5-(3-fluoropiperidin-4-yl)-4-methyl-8-oxa-2,5,6,12,16,17-hexaazatricyclo [11.3.1.0^{3,7}]heptadeca-1(16),3,6,13(17),14-pentaene, TFA salt (D97)

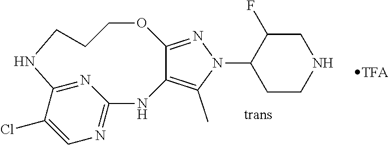 [SMILES: Cc1c2c(nn1C1CCNCC1F)OCCCNc1nc(ncc1Cl)N2]

To a solution of D96 (730 mg, ca. 1.51 mmol) in CH₂Cl₂ (20 mL) was added TFA (10 mL). The reaction was stirred at room temperature for 0.5 hour. The mixture was concentrated to give the title compound as brown oil (1.197 g, crude). LC-MS: 382.3 [M+H]⁺.

### Description D98

(R)-tert-Butyl 4-(3-(3-((2,5-dichloropyrimidin-4-yl)amino)butoxy)-5-methyl-4-nitro-1H-pyrazol-1-yl)piperidine-1-carboxylate (D98)

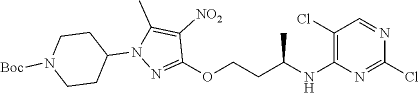 [SMILES: Cc1c([N+](=O)[O-])c(OCC[C@@H](C)Nc2nc(Cl)ncc2Cl)nn1C1CCN(C(=O)OC(C)(C)C)CC1]

A solution of D79 (1.0 g, 2.78 mmol), tert-butyl 4-bromopiperidine-1-carboxylate (2.2 g, 62.5 mmol) and Cs₂CO₃ (2.7 g, 8.33 mmol) in DMA (50 mL) was heated to 80° C. and stirred for 8 hrs. The reaction mixture was poured into water (250 mL) and extracted with EtOAc (3×100 mL). The combined organic layer was washed with brine, dried over anhydrous Na₂SO₄ and concentrated in vacuo. The crude product was purified by flash column chromatography (PE:EtOAc=5:1 to 3:1) to give product as a white solid. (700 mg yield 46%). LC-MS: 544.3 [M+H]⁺.

### Description D99

### tert-Butyl 4-[(11R)-14-chloro-4,11-dimethyl-8-oxa-2,5,6,12,16,17-hexaazatricyclo [11.3.1.0^{3,7}]heptadeca-1(16),3,6,13(17),14-pentaen-5-yl]piperidine-1-carboxylate (D99)

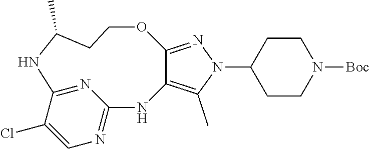 [SMILES: Cc1c2c(nn1C1CCN(C(=O)OC(C)(C)C)CC1)OCC[C@@H](C)Nc1nc(ncc1Cl)N2]

A solution of D98 (700 mg, 1.29 mmol), Fe (700 mg, 12.5 mmol) and NH₄Cl (1.4 g, 26.1 mmol) in EtOH and H₂O (30 mL, 3:1) was heated to 90° C. for 16 hrs. Water (5 mL) and NaHCO₃(200 mg) was added and the mixture was stirred for 10 min, filtered, washed with DCM and concentrated. The residue was re-dissolved in DCM (50 mL) and the mixture was stirred for 10 min, filtered and concentrated to give the title compound as a yellow solid (600 mg, yield 97%). LC-MS: 478.3 [M+H]⁺.

### Description D100

### (11R)-14-Chloro-4,11-dimethyl-5-(piperidin-4-yl)-8-oxa-2,5,6,12,16,17-hexaazatri-cyclo [11.3.1.0^{3,7}]heptadeca-1(16),3,6,13(17),14-pentaene, HCl salt (D100)

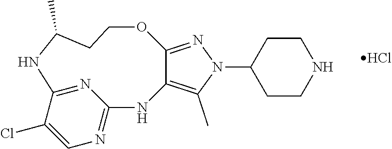 [SMILES: Cc1c2c(nn1C1CCNCC1)OCC[C@@H](C)Nc1nc(ncc1Cl)N2 ; Cl]

A solution of D99 (600 mg, 1.1 mmol) in EtOH/HCl (25 mL) was stirred at room temperature for 16 hrs. The mixture was concentrated to give the title compound as a yellow solid (500 mg, yield 94%). LC-MS: 378.3. [M+H]⁺.

### Description D102

### Methyl 3-(dibenzylamino)-2-fluorobutanoate (D102)

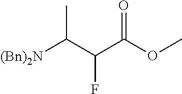

To a solution of methyl 3-(dibenzylamino)-2-hydroxybutanoate (16.7 g, 53.29 mmol) in THF (220 mL) at room temperature was added dropwise the solution of DAST (12.0 g, 74.60 mmol) in THF (20 mL). The reaction was stirred overnight at room temperature. The mixture was poured into sat.NaHCO₃(200 mL) and extracted with EtOAc (3×80 mL). The combined organic layers were washed with water (80 mL), brine (50 mL), dried over Na₂SO₄ and concentrated. The crude was purified by column chromatography on silica gel (PE:EtOAc=80:1) to give the title compound as a yellow solid (9.9 g, yield 59%). ¹H NMR (400 MHz, CDCl₃): δ 7.26˜7.05 (m, 10H), 4.77 (dd, J=49.1, 3.7 Hz, 1H), 3.83 (d, J=13.4 Hz, 2H), 3.56 (s, 3H), 3.31˜3.23 (m, 2H), 3.18 (ddd, J=14.0, 7.0, 3.9 Hz, 1H), 1.20 (d, J=7.0 Hz, 3H).

### Description D103

### 3-(Dibenzylamino)-2-fluorobutan-1-ol (D103)

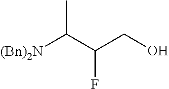

To a suspension of LiBH₄ (2.1 g, 94.2 mmol) in THF (110 mL) at −10° C. was added dropwise the solution of D102 (9.9 g, 31.4 mmol) in THF (20 mL). The reaction was warmed to room temperature and stirred overnight. The mixture was poured into sat.NH₄Cl (200 mL) and extracted with EA (3×100 mL). The combined organic layers were washed with water (100 mL), brine (50 mL), dried over Na₂SO₄, and concentrated. The crude was purified by column chromatography (PE:EtOAc=10:1) to give the title compound as a colorless oil (8.0 g, yield 89%). ¹H NMR (400 MHz, CDCl₃): δ 7.41˜7.07 (m, 10H), 4.41˜4.20 (m, 1H), 3.91 (d, J=13.3 Hz, 2H), 3.82˜3.51 (m, 3H), 3.29 (d, J=13.3 Hz, 2H), 2.93 (dqd, J=30.3, 7.0, 3.8 Hz, 1H), 1.17 (d, J=8.9 Hz, 3H).

### Description D104

### 3-Amino-2-fluorobutan-1-ol (D104)

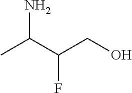

To a solution of D103 (1.5 g, 5.22 mmol) in MeOH (180 mL) was added Pd/C (10%, 0.3 g).

The mixture was stirred at 45° C. under 1 atm of hydrogen overnight. The mixture filtered and the filtrate was concentrated to give the title compound as colorless oil (0.425 g, yield 76%). ¹H NMR (400 MHz, CDCl₃): δ 4.44˜4.19 (m, 1H), 4.02˜3.65 (m, 2H), 3.35˜3.12 (m, 1H), 2.21 (m, 3H), 1.28˜1.14 (d, J=6.4 Hz, 3H).

### Description D105

### 3-((2,5-Dichloropyrimidin-4-yl)amino)-2-fluorobutan-1-ol (D105)

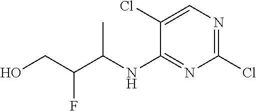

To a solution of D104 (425 mg, 3.96 mmol) in i-PrOH (10 mL) was added 2,4,5-trichloropyrimidine (728 mg, 3.96 mmol). The mixture was cooled to 0° C. and DIPEA (2.3 mL, 14.28 mmol) was added. The reaction was stirred at 0° C. for 5 min, then warmed to room temperature and stirred overnight. The mixture was diluted with DCM (30 mL) and washed with water (30 mL). The aqueous layer was further extracted with DCM (2×30 mL).

The combined organic layers were washed with brine (30 mL), dried over Na₂SO₄, concentrated. The crude was purified by column chromatography on silica gel (PE:EtOAc=4:1) to give the title compound as a white solid (800 mg, yield 80%). ¹H NMR (400 MHz, CDCl₃): δ 8.02 (s, 1H), 5.41 (m, 1H), 4.66˜4.26 (m, 2H), 3.76˜3.62 (m, 1H), 3.47 (m, 1H), 3.06˜3.02 (m, 1H), 1.36 (d, J=6.8 Hz, 3H).

### Description D106

### 2,5-Dichloro-N-(3-fluoro-4-((5-methyl-4-nitro-1-(tetrahydro-2H-pyran-4-yl)-1H-pyrazol-3-yl)oxy)butan-2-yl)pyrimidin-4-amine (D106)

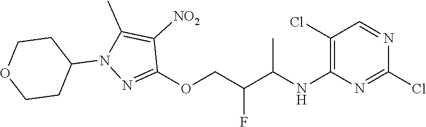 [SMILES: Cc1c([N+](=O)[O-])c(OCC(F)C(C)Nc2nc(Cl)ncc2Cl)nn1C1CCOCC1]

To a solution of D105 (290 mg, 1.14 mmol) in THF (20 mL) were added D5 (259 mg, 1.14 mmol) and Ph₃P (598 mg, 2.28 mol). After cooled to 0° C., DEAD (397 mg, 2.28 mmol) was added dropwise at 0° C. The reaction was stirred at 0° C. for 1 hour, then warmed to room temperature and stirred for 2 hrs. The mixture was diluted with EA (30 mL) and washed with water (30 mL). The aqueous layer was further extracted with EA (2×20 mL). The combined organic layers were washed with water (30 mL), brine (30 mL), dried over Na₂SO₄, and concentrated. The crude was purified by column chromatography on silica gel (DCM:MeOH=4:1) and then pre-TLC (PE:EtOAc=1:2) to give the title compound as a white solid (170 mg, yield 32%). LC-MS: 463.1 [M+H]⁺.

### Description D107

### 5-Bromo-2-chloro-N-(3-((5-methyl-4-nitro-1H-pyrazol-3-yl)oxy)propyl)pyrimidin-4-amine (D107)

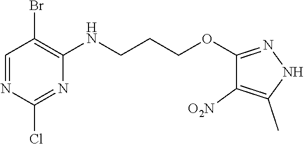 [SMILES: Cc1[nH]nc(OCCCNc2nc(Cl)ncc2Br)c1[N+](=O)[O-]]

To a solution of D32 (500 mg, 2.11 mmol) in i-PrOH (15 ml) was added DIPEA (1.4 mL, 8.451 mmol). After stirred under argon at 0° C. for 10 min and 5-bromo-2,4-dichloropyrimidine (0.27 mL, 2.324 mmol) was added via syringe. The reaction was warmed gradually to room temperature and stirred under argon for overnight. The mixture was concentrated and the residue was suspended in i-PrOH (1 ml) and water (5 mL) and stirred at room temperature for 0.5 hour, filtered, dried and concentrated to give the title compound as an off-white solid (780 g, yield 94%). LC-MS: 393.1 [M+H]⁺. ¹H NMR (400 MHz, DMSO-d₆): δ 12.93 (br, 1H), 8.22 (s, 1H), 7.80 (t, J=5.2 Hz, 1H), 4.28 (t, J=6.0 Hz, 2H), 3.53 (q, J=6.5 Hz, 2H), 2.47 (s, 3H), 2.07˜2.01 (m, 2H).

### Description D108

### 5-Bromo-2-chloro-N-(3-((5-methyl-4-nitro-1-(oxetan-3-yl)-1H-pyrazol-3-yl)oxy) propyl)pyrimidin-4-amine (D108)

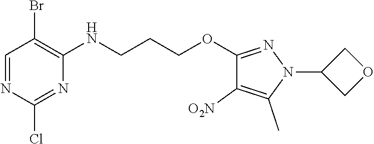 [SMILES: Cc1c([N+](=O)[O-])c(OCCCNc2nc(Cl)ncc2Br)nn1C1COC1]

To a solution of 3-bromooxetane (1.245 g, 9.09 mmol) in DMSO (15.0 mL) was added D107 (890 mg, 2.27 mmol) and K₂CO₃ (942 mg, 6.82 mmol). The reaction was stirred overnight at 100° C. The cooled mixture was diluted with water (150 mL) and extracted with EtOAc (3×80 mL). The combined organic layers were washed with brine, dried over anhydrous Na₂SO₄ and filtered. The filtrate was concentrated and the crude was purified by column chromatography on silica gel (PE:EtOAc=4:1) to give the title compound as a yellow solid (537 mg, yield 52%). LC-MS: 449.1 [M+H]⁺.

### Description D109

(R)-2,5-dichloro-N-(4-((5-methyl-4-nitro-1-(oxetan-3-yl)-1H-pyrazol-3-yl)oxy)butan-2-yl)pyrimidin-4-amine (D109)

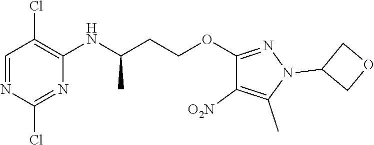 [SMILES: Cc1c([N+](=O)[O-])c(OCC[C@@H](C)Nc2nc(Cl)ncc2Cl)nn1C1COC1]

A solution of D79 (10.0 g, 27.66 mmol), 3-bromooxetane (13.7 g, 99.67 mmol) and K₂CO₃ (11.5 g, 82.98 mmol) in DMSO (100 mL) was heated to 80° C. and stirred for 16 hrs. The mixture was poured into ice water (300 mL) and extracted with EtOAc (5×200 mL). The organic layers were washed with brine, dried over anhydrous Na₂SO₄ and concentrated. The crude was purified by chromatography on silica gel (PE:EtOAc=1:3) to give the title compound (6.5 g, crude). LC-MS: 417.2 [M+H]⁺.

### Description D110

### tert-Butyl 3-(3-((R)-3-((2,5-dichloropyrimidin-4-yl)amino)butoxy)-5-methyl-4-nitro-1H-pyrazol-1-yl)piperidine-1-carboxylate (D110)

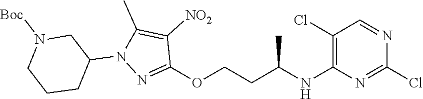 [SMILES: Cc1c([N+](=O)[O-])c(OCC[C@@H](C)Nc2nc(Cl)ncc2Cl)nn1C1CCCN(C(=O)OC(C)(C)C)C1]

A solution of D79 (1.0 g, 2.78 mmol), tert-butyl 3-((methylsulfonyl)oxy)piperidine-1-carboxylate (2.3 g, 8.33 mmol) and Cs₂CO₃ (2.7 g, 8.33 mmol) in DMA (25 mL) was heated to 80° C. and stirred for 48 hrs. The mixture was poured into water (150 mL) and extracted with EtOAc (3×150 mL). The combined organic layers were washed with brine, dried over anhydrous Na₂SO₄ and concentrated. The crude was purified by flash column chromatography on silica gel (PE:EtOAc=5:1 to 3:1) to give the title compound as a white solid (1.6 g, yield 100%). LC-MS: 544.2 [M+H]⁺.

### Description D111

### tert-Butyl 3-[(11R)-14-chloro-4,11-dimethyl-8-oxa-2,5,6,12,16,17-hexaazatricyclo-[11.3.1.0^{3,7}]heptadeca-1(16),3,6,13(17),14-pentaen-5-yl]piperidine-1-carboxylate (D111)

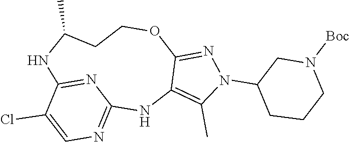 [SMILES: Cc1c2c(nn1C1CCCN(C(=O)OC(C)(C)C)C1)OCC[C@@H](C)Nc1nc(ncc1Cl)N2]

A solution of D110 (1.6 g, 2.95 mmol), Fe (1.6 mg, 28.5 mmol) and NH₄Cl (3.2 g, 59.8 mmol) in EtOH and H₂O (50 mL, 3:1) was heated to 90° C. for 16 hrs. water (5 mL) and sat.NaHCO₃ (500 mg) was added and the mixture was stirred for 10 min, filtered, washed with DCM and concentrated. The residue was re-dissolved in DCM (50 mL) and the mixture was stirred for 30 min, filtered and concentrated to give the title compound as a little yellow solid (1.3 g, yield 92%). LC-MS: 478.3 [M+H]⁺.

### Description D112

### (11R)-14-chloro-4,11-dimethyl-5-(piperidin-3-yl)-8-oxa-2,5,6,12,16,17-hexaaza tricyclo[11.3.1.0^{3,7}]heptadeca-1(16),3,6,13(17),14-pentaene, HCl salt (D112)

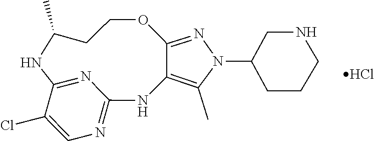 [SMILES: Cc1c2c(nn1C1CCCNC1)OCC[C@@H](C)Nc1nc(ncc1Cl)N2 ; Cl]

A solution of D111 (1.3 g, 3.45 mmol) in EtOH/HCl (100 mL) was stirred for 5 hrs at room temperature. The mixture was concentrated to give the title compound as a yellow solid (800 mg, yield 80%). LC-MS: 378.3 [M+H]⁺.

### Description D113

### 2-Chloro-44(3-((5-methyl-4-nitro-1-(tetrahydro-2H-pyran-4-yl)-1H-pyrazol-3-yl)oxy) propyl)amino)pyrimidine-5-carbonitrile (D113)

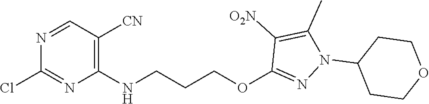 [SMILES: Cc1c([N+](=O)[O-])c(OCCCNc2nc(Cl)ncc2C#N)nn1C1CCOCC1]

To a solution of D7 (100 mg, 0.35 mmol) in i-PrOH (10 mL) were added Et₃N (106.77 mg) and 2,4-dichloropyrimidine-5-carbonitrile (61.2 mg, 0.35 mmol). The reaction was stirred at 30° C. for 1 hour. The mixture was poured into water (50 mL) and extracted with CH₂Cl₂ (3×50 mL). The organic layer was dried over anhydrous Na₂SO₄ and concentrated to give the title compound as colorless oil (100 mg, yield 66%). LC-MS: 422.2 [M+H]⁺.

### Description D114

### tert-Butyl (3-((1-(3-fluorotetrahydro-2H-pyran-4-yl)-5-methyl-4-nitro-1H-pyrazol-3-yl) oxy)-2-methoxypropyl)carbamate (D114)

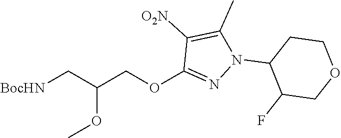 [SMILES: COC(CNC(=O)OC(C)(C)C)COc1nn(C2CCOCC2F)c(C)c1[N+](=O)[O-]]

To a solution of D50 (350 mg, 1.43 mmol) in DMF (10 mL) were added K₂CO₃ (591 mg, 4.28 mmol) and D13 (606 mg, 2.14 mmol). The reaction was stirred at 80° C. for 5 hrs. The mixture was poured into water (50 mL) and extracted with EtOAc (3×50 mL). The organic layers were concentrated and the crude was purified by column chromatography on silica gel (PE:EtOAc=1:1) to give the title compound as a yellow solid (300 mg, yield 50%). LC-MS: 465.3 [M+Na]⁺.

### Description D115

### 3-((1-(3-Fluorotetrahydro-2H-pyran-4-yl)-5-methyl-4-nitro-1H-pyrazol-3-yl)oxy)-2-methoxypropan-1-amine, TFA salt (D115)

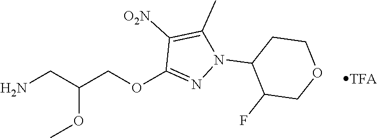 [SMILES: COC(CN)COc1nn(C2CCOCC2F)c(C)c1[N+](=O)[O-]]

To a solution of D114 (300 mg) in CH₂Cl₂ (15 mL) were added TFA (5 mL). The reaction was stirred at 25° C. for 0.5 hour. The mixture was concentrated to give the title compound as yellow oil (200 mg, yield 87%). LC-MS: 333.3 [M+H]⁺.

### Description D116

### 2,5-Dichloro-N-(3-((1-(3-fluorotetrahydro-2H-pyran-4-yl)-5-methyl-4-nitro-1H-pyrazol-3-yl)oxy)-2-methoxypropyl)pyrimidin-4-amine (D116)

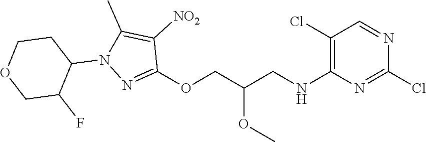 [SMILES: COC(CNc1nc(Cl)ncc1Cl)COc1nn(C2CCOCC2F)c(C)c1[N+](=O)[O-]]

To a solution of D115 (200 mg, 0.6 mmol) in i-PrOH (10 mL) were added Et₃N (182.69 mg) and 2,4,5-trichloropyrimidine (111 mg, 0.6 mmol). The reaction was stirred at 30° C. for 1 hour. The reaction was concentrated and the the crude was purified by pre-TLC to give the title compound as a yellow solid (250 mg, yield 86%) LC-MS: 479.2 [M+H]⁺.

### Description D117

### 2,5-Dichloro-N-(4-((5-methyl-1-(2-methyltetrahydro-2H-pyran-4-yl)-4-nitro-1H-pyrazol-3-yl)oxy)butan-2-yl)pyrimidin-4-amine (D117)

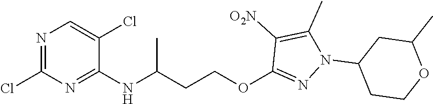 [SMILES: Cc1c([N+](=O)[O-])c(OCCC(C)Nc2nc(Cl)ncc2Cl)nn1C1CCOC(C)C1]

To a solution of 2-methyltetrahydro-2H-pyran-4-yl methanesulfonate (582 mg, 3 mmol) in DMSO (20 mL) were added K₂CO₃ (414 mg, 3 mmol) and D73 (361 mg, 1 mmol). The reaction was stirred at 80° C. overnight. The mixture was poured into water (100 mL) and extracted with EtOAc (2×80 mL). The organic layer was washed with brine, dried over anhydrous Na₂SO₄ and concentrated in vacuo. The product was purified by flash column chromatography on silica gel (PE:EtOAc=4:1) to give the title compound as a yellow solid (160 mg, yield 34%). LC-MS: 459.2 [M+H]⁺.

### Description D118

### 3-(3-(3-((2,5-Dichloropyrimidin-4-yl)amino)propoxy)-5-methyl-4-nitro-1H-pyrazol-1-yl)dihydro-2H-pyran-4(3H)-one (D118)

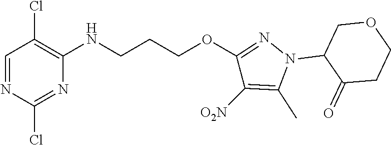 [SMILES: Cc1c([N+](=O)[O-])c(OCCCNc2nc(Cl)ncc2Cl)nn1C1COCCC1=O]

To a solution of D33 (1.0 g, 2.88 mmol) in DMF (10 mL) were added K₂CO₃ (636 mg, 4.60 mmol) and 3-bromodihydro-2H-pyran-4(3H)-one (825 mg, 4.61 mmol). The reaction was stirred at 25° C. for 16 hrs. The mixture was poured into aq. NH₄Cl (40 mL) and extracted with EtOAc (2×40 mL). The combined organics were washed with brine, dried over anhydrous Na₂SO₄ and concentrated in vacuo. The crude was purified by flash column chromatography on silica gel (PE:EtOAc=10:1 to 3:1) to give the title compound as a yellow solid (1.0 g, yield 78%). LC-MS: 445.2 [M+H]⁺.

### Description D119

### 3-(3-(3-((2,5-Dichloropyrimidin-4-yl)amino)propoxy)-5-methyl-4-nitro-1H-pyrazol-1-yl)tetrahydro-2H-pyran-4-ol (D119)

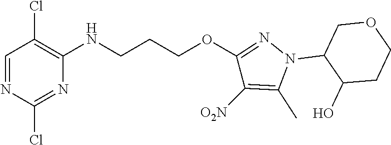 [SMILES: Cc1c([N+](=O)[O-])c(OCCCNc2nc(Cl)ncc2Cl)nn1C1COCCC1O]

To a solution of D118 (1.0 g, 2.25 mmol) in MeOH (15 mL) at 0° C. was added NaBH₄ (170 mg, 4.49 mmol) slowly at 0° C. The reaction was stirred at 25° C. for 2 hrs. The mixture was poured into aq. HCl (0.5M, 20 mL) and extracted with EtOAc (3×15 mL). The combined organic layers were washed with brine, dried over anhydrous Na₂SO₄ and concentrated. The crude was purified by column chromatography on silica gel (PE:EtOAc=10:1 to 2:1) to give the title compound as yellow oil (700 mg, yield 69.9%). LC-MS: 447.1 [M+H]⁺.

### Description D120

### 2,5-Dichloro-N-(3-((1-(4-fluorotetrahydro-2H-pyran-3-yl)-5-methyl-4-nitro-1H-pyrazol-3-yl)oxy)propyl)pyrimidin-4-amine (D120)

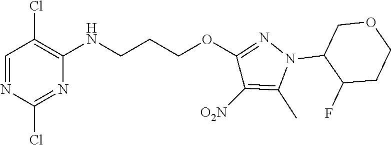 [SMILES: Cc1c([N+](=O)[O-])c(OCCCNc2nc(Cl)ncc2Cl)nn1C1COCCC1F]

To a solution of D119 (700 mg, 1.565 mmol) in CH₂Cl₂(14 mL) at 0° C. was added DAST (1.0 g, 6.26 mmol) slowly. The reaction was stirred at 25° C. overnight. The mixture was poured into aq. Na₂CO₃ (15 mL) and extracted with CH₂Cl₂ (2×20 mL). The combined organic layers were washed with brine, dried over anhydrous Na₂SO₄ and concentrated. The crude was purified by column chromatography on silica gel (PE:EtOAc=10:1 to 4:1) to give the target product as yellow oil (230 mg, yield 32.7%). LC-MS: 449.1 [M+H]⁺.

### Description D121

### Ethyl 4,4-difluoro-3-oxobutanoate (D121)

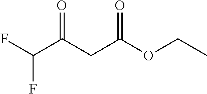

To a slurry of sodium hydride (60%, 1440 mg, 36.0 mmol) in THF (30 mL) was added ethyl acetate (2.94 mL, 30.0 mmol) followed by the addition of ethyl 2,2-difluoroacetate (3.60 mL, 36.0 mmol). The reaction was stirred at 40° C. overnight. The mixture was then cooled to 0° C., and quenched with HCl (2 N) solution until pH=6˜7. The mixture was stirred at room temperature for 20 min, diluted with EtOAc (40 mL), washed with aq.NaHCO₃ (2×40 mL) and brine (2×40 mL). The organic layer was dried over anhydrous Na₂SO₄ and concentrated to give the crude as yellow oil, which was used in next step without further purification.

### Description D122

### Ethyl 3-(benzylimino)-4,4-difluorobutanoate (D122)

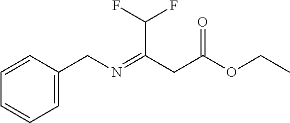 [SMILES: CCOC(=O)C/C(=N/Cc1ccccc1)C(F)F]

To a solution of phenylmethanamine (3.45 mL, 31.6 mmol) in EtOH (40 mL) were added D121 (3.500 g, 21.07 mmol) and acetic acid (1.809 mL, 31.6 mmol). The reaction was stirred at room temperature for 1 hour. The mixture was then diluted with EtOAc (100 mL), washed with aq. NaHCO₃(2×80 mL) and brine (2×80 mL). The organic layer was dried over anhydrous Na₂SO₄ and concentrated. The crude was purified by column chromatography on silica gel (PE:EtOAc=1:0 to 5:1) to give the title compound as yellow oil (2860 mg, yield 53.2%). ¹H NMR (600 MHz, CDCl₃): δ 7.32˜7.18 (m, 5H), 5.94 (t, J=53.4 Hz, 1H), 4.45 (d, J=6.6 Hz, 2H), 4.13 (d, J=5.4 Hz, 2H), 4.06 (q, J=7.2, 2H), 1.19 (t, J=7.2 Hz, 3H).

### Description D123

### 3-(Benzylamino)-4,4-difluorobutan-1-ol (D123)

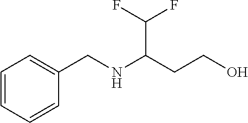 [SMILES: OCCC(NCc1ccccc1)C(F)F]

To a slurry of LiAlH₄ (5.88 mL, 11.75 mmol, 2 M in THF), D122 (1.0 g, 3.92 mmol) in THF (2 mL) was added dropwise at 0° C. The reaction was kept stirring at room temperature for 3 hrs and then quenched with brine (2 mL). The mixture was filtered and the filtrate was diluted with EtOAc (50 mL), washed with brine (20 mL). The organic layer was dried over anhydrous Na₂SO₄, filtered and concentrated. The crude was purified by 018 chromatography (5-95% CH₃CN in water) to give the title compound as colorless oil (370 mg, yield 43.9%). ¹H NMR (600 MHz, CDCl₃): δ 7.38˜7.29 (m, 5H), 5.84 (td, J=56.1, 3.0 Hz, 1H), 3.98 (dd, J=84.7, 12.8 Hz, 2H), 3.83 (m, 2H), 3.13 (m, 1H), 3.00 (br, 1H), 1.76 (m, 2H).

### Description D124

### 3-Amino-4,4-difluorobutan-1-ol (D124)

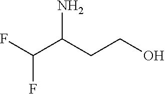

To a solution of D123 (508 mg, 2.360 mmol) in methanol (5 mL) was added Pd/C (126 mg, 0.118 mmol). The reaction was stirred overnight at room temperature under hydrogen. The mixture was filtered and the filtrate was concentrated to give the title compound as colorless oil, which was used in next step without purification.

### Description D125

### 3-((2,5-Dichloropyrimidin-4-yl)amino)-4,4-difluorobutan-1-ol (D125)

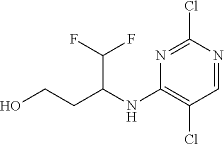 [SMILES: OCCC(Nc1nc(Cl)ncc1Cl)C(F)F]

To a solution of D124 (296 mg, 2.366 mmol) in DMF (6 mL) were added 2,4,5-trichloropyrimidine (0.325 mL, 2.84 mmol) and DIPEA (0.618 mL, 3.55 mmol). The reaction was stirred overnight at room temperature. The mixture was diluted with EtOAc (50 mL), washed with water (3×30 mL) and brine (30 mL). The organic layer was dried over anhydrous Na₂SO₄ and concentrated. The crude was purified by column chromatography on silica gel (PE:EtOAc=1:0 to 0:1) to give the title compound as a as white solid (500 mg, yield 73.6%). ¹H NMR (400 MHz, DMSO-d₆): δ 8.27 (s, 1H), 7.85 (d, J=8.6 Hz, 1H), 6.09 (td, J=55.7, 3.7 Hz, 1H), 4.68 (m, 1H), 4.63 (t, J=4.9 Hz, 1H), 3.46 (m, 2H), 1.87 (m, 2H).

### Description D126

### 3-((2,5-Dichloropyrimidin-4-yl)amino)-4,4-difluorobutyl methanesulfonate (D126)

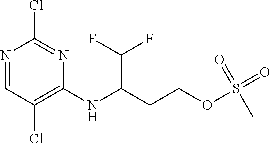 [SMILES: CS(=O)(=O)OCCC(Nc1nc(Cl)ncc1Cl)C(F)F]

To a solution of D125 (500 mg, 1.838 mmol) in THF (4 mL) was added DIPEA (0.480 mL, 2.76 mmol) followed by the addition of methanesulfonyl chloride (0.171 mL, 2.205 mmol).

The reaction was stirred at room temperature for 30 min. The mixture was then quenched with water (0.2 mL), diluted with EtOAc (40 mL), washed with water (2×30 mL) and brine (30 mL). The organic layer was dried over anhydrous Na₂SO₄ and concentrated to give the crude as yellow oil, which was used in next step without further purification.

### Description D127

### 2,5-Dichloro-N-(1,1-difluoro-4-((5-methyl-4-nitro-1-(tetrahydro-2H-pyran-4-yl)-1H-pyrazol-3-yl)oxy)butan-2-yl)pyrimidin-4-amine (D127)

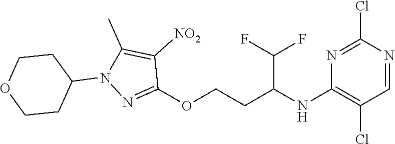 [SMILES: Cc1c([N+](=O)[O-])c(OCCC(Nc2nc(Cl)ncc2Cl)C(F)F)nn1C1CCOCC1]

To a solution of D126 (280 mg, 0.800 mmol) in DMF (2 mL) were added D5 (182 mg, 0.800 mmol) and Cs₂CO₃ (287 mg, 0.880 mmol). The reaction was heated to 60° C. in a sealed system and stirred for 1 hour. After cooled to room temperature, the mixture was diluted with EtOAc (20 mL), washed with water (3×20 mL) and brine (20 mL). The organic layer was dried over anhydrous Na₂SO₄ and concentrated. The crude was purified by column chromatography on silica gel (PE:EtOAc=1:0 to 0:1) to give the title compound as a white solid (112 mg, yield 18.76%). LC-MS: 481.1 [M+H]⁺.

### Description D128

### tert-Butyl 4-(3-(3-((2,5-dichloropyrimidin-4-yl)amino)butoxy)-5-methyl-4-nitro-1H-pyrazol-1-yl)-3-fluoropiperidine-1-carboxylate (D128)

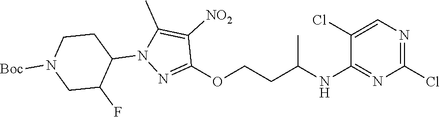 [SMILES: Cc1c([N+](=O)[O-])c(OCCC(C)Nc2nc(Cl)ncc2Cl)nn1C1CCN(C(=O)OC(C)(C)C)CC1F]

A solution of D73 (723 mg, 2.0 mmol), tert-butyl 3-fluoro-4-((methylsulfonyl)oxy) piperidine-1-carboxylate (2.97 g, 10.0 mmol) and Cs₂CO₃ (1.96 g, 6.0 mmol) in DMSO (30 mL) was stirred at 85° C. overnight. The mixture was poured into the sat.NaCl (aq.) and extracted with EtOAc (3×30 mL). The combined organic layers were washed with brine, dried over anhydrous Na₂SO₄ and concentrated in vacuo. The crude was purified by column chromatography on silica gel (PE:EtOAc=1:1) to give the title compound as a yellow solid (280 mg, yield 25%).

### Description D129

### tert-Butyl 4-{14-chloro-4,11-dimethyl-8-oxa-2,5,6,12,16,17-hexaazatricyclo[11.3.1.0^{3,7}]heptadeca-1(16),3,6,13(17),14-pentaen-5-yl}-3-fluoropiperidine-1-carboxylate (D129)

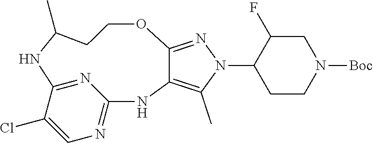 [SMILES: Cc1c2c(nn1C1CCN(C(=O)OC(C)(C)C)CC1F)OCCC(C)Nc1nc(ncc1Cl)N2]

A solution of D129 (571 mg, 1.1 mmol), Fe (285 mg, 5.1 mmol) and NH₄Cl (273 mg, 5.1 mmol) in EtOH/H₂O (24 mL, 5:1) was refluxed overnight. The mixture was filtered and the filtrate was concentrated. The crude was purified by column chromatography on silica gel (PE:EtOAc=2:1) to give the title compound as a white solid (175 mg, yield 34.6%).

### Description D130

### 14-Chloro-5-(3-fluoropiperidin-4-yl)-4,11-dimethyl-8-oxa-2,5,6,12,16,17-hexaazatri cyclo[11.3.1.0^{3,7}]heptadeca-1(16),3,6,13(17),14-pentaene, TFA salt (D130)

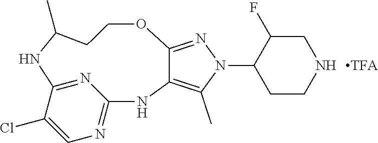 [SMILES: Cc1c2c(nn1C1CCNCC1F)OCCC(C)Nc1nc(ncc1Cl)N2 ; "·TFA"]

A solution of D129 (175 mg, 0.35 mmol) and TFA (2.5 mL) in DCM (15 mL) was stirred at room temperature for 5 hrs. The mixture was concentrated to give the crude (135 mg), which was used for the next step directly.

### Description D131

### tert-Butyl 4-(3-((R)-3-((2,5-dichloropyrimidin-4-yl)amino)butoxy)-5-methyl-4-nitro-1H-pyrazol-1-yl)-3-fluoropiperidine-1-carboxylate (D131)

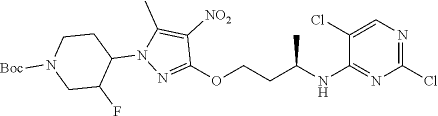 [SMILES: Cc1c([N+](=O)[O-])c(OCC[C@@H](C)Nc2nc(Cl)ncc2Cl)nn1C1CCN(C(=O)OC(C)(C)C)CC1F]

A solution of D79 (1.08 mg, 3.0 mmol), tert-butyl 3-fluoro-4-((methylsulfonyl)oxy) piperidine-1-carboxylate (3.57 g, 12.0 mmol) and K₂CO₃(1.66 g, 12.0 mmol) in DMSO (30 mL) was stirred at 85° C. for 3 days. The mixture was poured into water and extracted with EtOAc (3×50 mL). The combined organic layer was dried over anhydrous Na₂SO₄ and concentrated in vacuo. The crude was purified by column chromatography on silica gel (PE:EtOAc=2:1) to give the title compound as a yellow solid (1.2 g, yield 71.0%).

### Description D132

### tert-Butyl 4-[(11R)-14-chloro-4,11-dimethyl-8-oxa-2,5,6,12,16,17-hexaazatricyclo [11.3.1.0^{3,7}]heptadeca-1(16),3,6,13(17),14-pentaen-5-yl]-3-fluoropiperidine-1-carboxylate (D132)

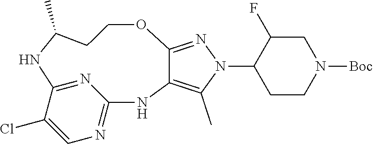 [SMILES: Cc1c2c(nn1C1CCN(C(=O)OC(C)(C)C)CC1F)OCC[C@@H](C)Nc1nc(ncc1Cl)N2]

A solution of D131 (5.3 g, 9.5 mmol), Fe (2.6 g, 47.5 mmol) and NH₄Cl (2.6 g, 47.5 mmol) in EtOH/H₂O (60 mL, 5:1) was refluxed overnight. The mixture was filtered and the filtrate was concentrated. The crude was purified by column chromatography on silica gel (PE:EtOAc=2:1) to give the title compound as a white solid (4.0 g, yield 85.1%).

### Description D133

### (11R)-14-Chloro-5-(3-fluoropiperidin-4-yl)-4,11-dimethyl-8-oxa-2,5,6,12,16,17-hexaazatricyclo[11.3.1.0^{3,7}]heptadeca-1(16),3,6,13(17),14-pentaene, TFA salt (D133)

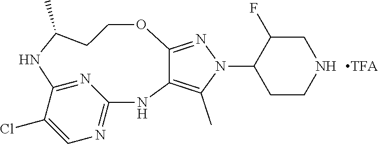 [SMILES: Cc1c2c(nn1C1CCNCC1F)OCC[C@@H](C)Nc1nc(ncc1Cl)N2 ; "·TFA"]

A solution of D132 (4.0 g, 8.1 mmol) and TFA (25 mL) in DCM (50 mL) was stirred at ambient temperature for 3 hours. The solvent was removed by vacuo and the crude desired product (3.1 g, crude) was used for the next step directly.

### Description D134

(R)-3-(3-(3-((2,5-Dichloropyrimidin-4-yl)amino)butoxy)-5-methyl-4-nitro-1H-pyrazol-1-yl)cyclobutan-1-one (D134)

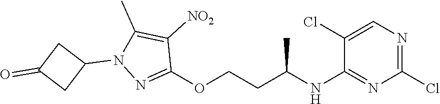 [SMILES: Cc1c([N+](=O)[O-])c(OCC[C@@H](C)Nc2nc(Cl)ncc2Cl)nn1C1CC(=O)C1]

To a solution of D79 (1 g, 2.8 mmol) in DMF (50 mL) was added NaH (124 mg, 3.1 mmol) at 0° C. After stirred for 30 min, 3-bromocyclobutan-1-one (834.3 mg, 5.6 mmol) was added and the mixture was stirred at room temperature for 4 hrs. Then the mixture was poured into ice water and extracted with EtOAc (3×100 mL). The combined organic layers were washed with brine, dried over anhydrous Na₂SO₄ and concentrated in vacuo. The crude was purified by column chromatography on silica gel (PE:EtOAc=2:1) to give the title compound as an off-white solid (850 mg, yield 71%). LC-MS: 429.2 [M+H]⁺.

### Description D135

### 3-[(11R)-14-Chloro-4,11-dimethyl-8-oxa-2,5,6,12,16,17-hexaazatricyclo[11.3.1.0^{3,7}]heptadeca-1(16),3,6,13(17),14-pentaen-5-yl]cyclobutan-1-one (D135)

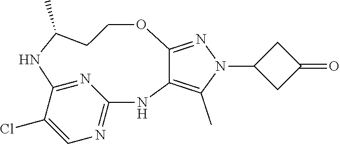 [SMILES: Cc1c2c(nn1C1CC(=O)C1)OCC[C@@H](C)Nc1nc(ncc1Cl)N2]

To a solution of D134 (850 mg, 1.98 mmol) in i-PrOH (50 mL) were added Fe (554.4 mg, 9.9 mmol) and a solution of NH₄Cl (529.3 mg, 9.9 mmol) in H₂O (5 mL). The reaction was stirred overnight at 95° C. The mixture was filtered and the filter cake was washed with CH₂C12/MeOH (10:1, 3×100 mL). The combined filtrate was concentrated. The residue was poured into sat.NaHCO₃ (aq.) and extracted with CH₂Cl₂ (3×100 mL). The combined organic layers were washed with brine, dried over anhydrous Na₂SO₄ and concentrated in vacuo. The crude was purified by column chromatography on silica gel (EtOAc) to give the title compound as an off-white solid (320 mg, yield 45%). LC-MS: 363.3 [M+H]⁺.

### Description D136

### 3-(3-Bromo-5-methyl-4-nitro-1H-pyrazol-1-yl)cyclopentanone (D136)

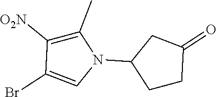 [SMILES: Cc1c([N+](=O)[O-])c(Br)cn1C1CCC(=O)C1]

To a stirred suspension of 3-bromo-5-methyl-4-nitro-1H-pyrazole (6.0 g, 29.1 mmol) and ScCl₃ (441 mg, 2.91 mmol) in CH₂Cl₂(60 mL) at room temperature was added cyclopent-2-enone (5.74 g, 69.9 mmol). The reaction was stirred at 25° C. for 16 hrs. The mixture was poured into brine (50 mL) and extracted with CH₂Cl₂ (2×50 mL). The combined organic layers were washed with brine, dried over anhydrous Na₂SO₄ and concentrated. The crude was purified by column chromatography (PE:EtOAc=10:1 to 4:1) to give the title compound as a white solid (6.8 g, yield 81%). ¹H NMR (400 MHz, CDCl₃): δ 5.26˜5.21 (m, 1H), 2.86 (dd, J=18.4, 5.6 Hz, 1H), 2.73˜2.62 (m, 2H), 2.54 (s, 3H), 2.52˜2.33 (m, 3H).

### Description D137

### 4-(3-(3-Bromo-5-methyl-4-nitro-1H-pyrazol-1-yl)cyclopentyl)morpholine (D137)

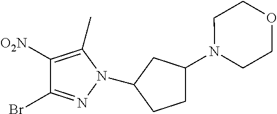 [SMILES: Cc1c([N+](=O)[O-])c(Br)nn1C1CCC(N2CCOCC2)C1]

To a stirred solution of D136 (500 mg, 1.736 mmol) and AcOH (36 mg, 0.607 mmol) in CH₂Cl₂ (10 mL) at 0° C. were added morpholine (163 mg, 1.909 mmol) and NaBH₃CN (218 mg, 3.471 mmol). The reaction was stirred at room temperature for 16 hrs. The mixture was washed with aq. NaHCO₃ (10 mL) and brine (10 mL). The organic layer was dried over anhydrous Na₂SO₄, filtered and concentrated. The crude was purified by column chromatography (PE:EtOAc=10:1 to 1:1) to give the title compound as yellow oil (318 mg, yield 51%). LC-MS: 361.2 [M+H]⁺.

### Description D138

### 5-Methyl-1-(3-morpholinocyclopentyl)-4-nitro-1H-pyrazol-3-ol (D138)

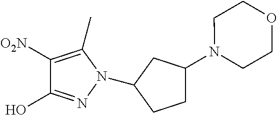 [SMILES: Cc1c([N+](=O)[O-])c(O)nn1C1CCC(N2CCOCC2)C1]

A solution of D137 (2.0 g, 5.57 mmol) and KOH (3.1 g, 55.6 mmol) in DME/water (20 mL/30 mL) in a sealed vessel was stirred at 120° C. for 16 hrs. Solvents were removed by concentration followed by lyophilization. The solid was suspended in methanol (20 mL), stirred at room temperature for 30 min. The resulting suspension was filtered through Celite.

The filtrate was concentrated and the crude was purified by column chromatography (CH₂Cl₂:CH₃OH=40:1 to 10:1) to give the title compound as a yellow solid (1.2 g, yield 72%). ¹H NMR (400 MHz, DMSO-d₆): δ 7.15 (br, 1H), 4.60˜4.57 (m, 1H), 4.76˜3.53 (m, 5H), 3.32˜2.50 (m, 4H), 2.21 (s, 3H), 2.17˜1.98 (m, 2H), 1.99˜1.60 (m, 4H).

### Description D139

### tert-Butyl (3-((5-methyl-1-(3-morpholinocyclopentyl)-4-nitro-1H-pyrazol-3-yl)oxy) prop yl)carbamate (D139)

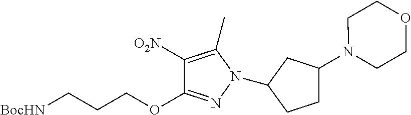 [SMILES: Cc1c([N+](=O)[O-])c(OCCCNC(=O)OC(C)(C)C)nn1C1CCC(N2CCOCC2)C1]

A mixture of D138 (300 mg, 1.01 mmol), tert-butyl (3-bromopropyl)carbamate (482 mg, 2.02 mmol) and Cs₂CO₃ (823 mg, 2.52 mmol) in DMF (6 mL) was stirred at 100° C. for 2 hrs. The reaction was quenched with water (10 mL) and extracted with ether (2×10 mL). The organic layer was washed with brine, dried over anhydrous Na₂SO₄, filtered and concentrated. The crude was purified by column chromatography on silica gel (CH₂Cl₂:CH₃OH=300:1 to 30:1) to give the title compound as yellow oil (220 mg, yield 48%). LC-MS: 454.3 [M+H]⁺.

### Description D140

### 3-((5-Methyl-1-(3-morpholinocyclopentyl)-4-nitro-1H-pyrazol-3-yl)oxy)propan-1-amine, TFA salt (D140)

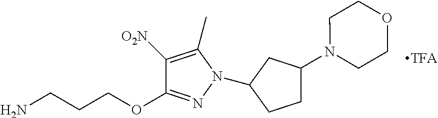 [SMILES: Cc1c([N+](=O)[O-])c(OCCCN)nn1C1CCC(N2CCOCC2)C1]

To a solution of D139 (220 mg, 0.485 mmol) in CH₂Cl₂ (2 mL) at 0° C. was added TFA (0.5 mL) dropwise. The reaction was stirred at room temperature for 2 hrs. The mixture was concentrated to give the title compound, which was used in next step without further purification.

### Description D141

### 2,5-Dichloro-N-(3-((5-methyl-1-(3-morpholinocyclopentyl)-4-nitro-1H-pyrazol-3-yl) oxy)propyl)pyrimidin-4-amine (D141)

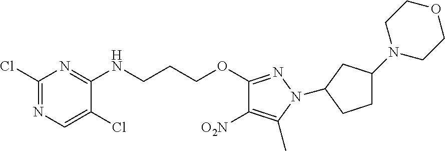 [SMILES: Cc1c([N+](=O)[O-])c(OCCCNc2nc(Cl)ncc2Cl)nn1C1CCC(N2CCOCC2)C1]

To a solution of D140 (170 mg, 0.481 mmol) in i-PrOH (2 mL) at 0° C. were added Et₃N (487 mg, 4.81 mmol) and 2,4,5-trichloropyrimidine (93 mg, 0.505 mmol) dropwise. The reaction was stirred at room temperature for 2 hrs. The mixture was poured into water (6 mL) and extracted with EtOAc (2×8 mL). The combined organic layer was dried over anhydrous Na₂SO₄ and concentrated in vacuo. The crude was purified by flash column chromatography (PE:EtOAc=10:1 to 0:1) to give the title compound as yellow oil (170 mg, yield 70%). LC-MS: 500.2 [M+H]⁺.

### Description D142

### 8-Chloro-3-methyl-2,4,10,11,12,13-hexahydro-5,9-(azeno)pyrazolo[3,4-b][1,4,6,10]oxatriazacyclotridecine (D142)

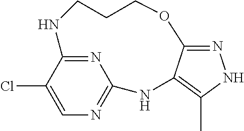 [SMILES: Cc1[nH]nc2c1Nc1ncc(Cl)c(n1)NCCCO2]

To a solution of D33 (150 mg, 0.432 mmol) in EtOH/H₂O (50 mL/5.0 mL) at room temperature were added Fe (125 mg, 2.24 mmol) and NH₄Cl (140 mg, 2.62 mmol). The reaction was stirred under argon at 100° C. overnight. The hot reaction suspension was filtered through a pad of Celite and the filtrate was concentrated. The crude was purified by chromatography on silica gel (CH₂Cl₂:MeOH=10:1) to give the title compound as a yellow solid (142 mg). LC-MS: 281.3 [M+H]⁺.

### Description D143

### 3,4,5-Tribromo-1-(tetrahydro-2H-pyran-4-yl)-1H-pyrazole (D143)

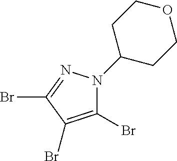 [SMILES: Brc1nn(C2CCOCC2)c(Br)c1Br]

To a solution of 3,4,5-tribromo-1H-pyrazole (10.500 g, 34.45 mmol), PPh3 (18.072 g, 68.90 mmol) and tetrahydro-2H-pyran-4-ol (4.222 g, 41.34 mmol) in THF (250 mL) was added DIAD (13.6 mL, 68.90 mmol) at 0° C. The mixture was stirred at 0° C. for 0.5 hour and at room temperature for 5 hrs. Then the reaction was poured into ice water (200 mL) and extracted with EtOAc (2×200 mL). The organic layers were dried, filtered and concentrated. The residue was purified by column chromatography on silica gel (PE:EtOAc=3:1) to give the title compound compound as a white solid (9.8 g, yield 73%).

### Description D144

### 3,4-Dibromo-1-(tetrahydro-2H-pyran-4-yl)-1H-pyrazole-5-carbaldehyde (D144)

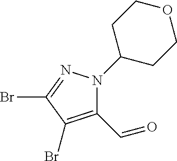 [SMILES: O=Cc1c(Br)c(Br)nn1C1CCOCC1]

To a solution of D143 (10.0 g, 25.71 mmol) in THF (200 mL) was added n-BuLi (1.6 M in THF, 24.1 mL, 38.57 mmol) at −78° C. under argon for 30 min. Then N-formylmorpholine (5.9 g, 51.42 mmol) was added drop wise. The reaction was stirred at −78° C. for 1 hour. The reaction was poured into aq NH₄Cl (200 mL), and extracted with EtOAc (2×100 mL). The combined organic layers were dried, filtered and concentrated. The residue was purified by column chromatography on silica gel (PE:EtOAc=10:1) to give the title compound as an off-white solid (5.2 g, yield 60%). ¹H NMR (400 MHz, CDCl₃): δ 9.83 (s, 1H), 5.27˜5.21 (m, 1H), 4.13˜4.07 (m, 2H), 3.57˜3.51 (m, 2H), 2.87˜2.18 (m, 2H), 1.91˜1.87 (m, 2H).

### Description D145

### 3,4-Dibromo-5-(difluoromethyl)-1-(tetrahydro-2H-pyran-4-yl)-1H-pyrazole (D145)

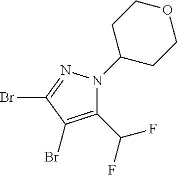 [SMILES: FC(F)c1c(Br)c(Br)nn1C1CCOCC1]

To a stirred solution of D144 (5.2 g, 15.38 mmol) in CH₂Cl₂ (100 mL) was added DAST (4.1 mL, 30.77 mmol) at 0° C. The reaction was stirred at room temperature for 2 hrs. The reaction was poured into ice. The mixture was extracted with CH₂Cl₂ (2×50 mL) and the combined organic layers were washed with aq.NaHCO₃ (2×100 mL), dried, filtered and concentrated to give the title compound as an gray solid (5.3 g, yield 96%). ¹H NMR (400 MHz, CDCl₃): δ 6.87˜6.61 (s, 1H), 4.61˜4.53 (m, 1H), 4.13˜4.09 (m, 2H), 3.54˜3.45 (m, 2H), 2.37˜2.37 (m, 2H), 1.88˜1.92 (m, 2H).

### Description D146

### 1-(4,4-Difluorocyclohexyl)-5-methyl-4-nitro-1H-pyrazol-3-ol (D146)

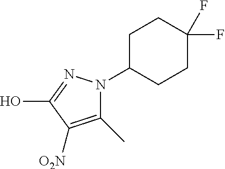 [SMILES: Cc1c([N+](=O)[O-])c(O)nn1C1CCC(F)(F)CC1]

The title compound D146 was prepared using procedures similar to those used for preparing D25, starting from 4,4-difluorocyclohexanol. LC-MS: 262.3 [M+H]⁺.

### Description D147

### 4-Nitro-1-(tetrahydrofuran-3-yl)-1H-pyrazol-3-ol (D147)

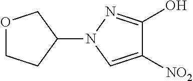 [SMILES: O=[N+]([O-])c1cn(C2CCOC2)nc1O]

The title compound D147 was prepared using procedures similar to those used for preparing D36, starting from tetrahydrofuran-3-yl methanesulfonate.

### Description D148

### 4-Nitro-3-(3-((tetrahydro-2H-pyran-2-yl)oxy)propoxy)-1-(tetrahydrofuran-3-yl)-1H-pyrazole (D148)

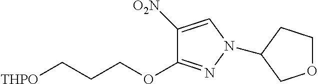

A mixture of D147 (1.00 g, 5.02 mmol), 2-(3-bromopropoxy)tetrahydro-2H-pyran (1.12 g, 5.02 mmol) and K₂CO₃ (1.39 g, 10.04 mmol) in DMF (20 mL) was heated at 80° C. for 3 hrs. The reaction mixture was poured into ice water (60 mL), and extracted with EtOAc (2×30 mL). The organic layer was washed with brine (3×40 mL), dried, filtered and concentrated. The crude was purified by column chromatography on silica gel (PE:EtOAc=1:1) to give the title compound as a yellow solid (1.526 g, yield 89%).

### Description D149

### 5-Chloro-4-nitro-3-(3-((tetrahydro-2H-pyran-2-yl)oxy)propoxy)-1-(tetrahydrofuran-3-yl)-1H-pyrazole (D149)

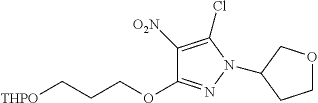

To a solution of D148 (1.526 g, 4.47 mmol) in THF (20 mL) were added LiHMDS (1.0 M in THF, 5.36 mL, 5.36 mmol) at −60° C. under Nitrogen. The reaction was stirred at −60° C. for 0.5 h. Then, a solution of 02016 (2.116 g, 8.94 mmol) in THF (2 mL) was added dropwise. The reaction was stirred at −60° C. for 0.5 hour. The reaction mixture was poured into aq.NH₄Cl (50 mL) and extracted with EtOAc (2×30 mL). The combined organic layer was washed with brine (2×30 mL), dried and concentrated in vacuo. The crude was purified by column chromatography on silica gel (PE:EtOAc=1:1) to give the title compound as a white solid (1.149 g, yield 68%).

### Description D150

### 3-((5-Chloro-4-nitro-1-(tetrahydrofuran-3-yl)-1H-pyrazol-3-yl)oxy)propan-1-ol (D150)

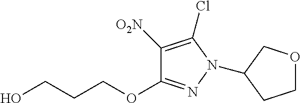 [SMILES: O=[N+]([O-])c1c(OCCCO)nn(C2CCOC2)c1Cl]

A solution of D149 (1.149 g, 3.06 mmol) in HCl/MeOH (3 M in MeOH, 15 mL) was heated at 50° C. for 1 hour. The reaction mixture was poured into ice and aq.NaHCO₃(50 mL), and then extracted with EtOAc (2×50 mL). The organic layer was dried, filtered and concentrated to give the title compound as yellow oil (734 mg, yield 82%).

### Description D151

### 3-((5-Chloro-4-nitro-1-(tetrahydrofuran-3-yl)-1H-pyrazol-3-yl)oxy)propyl methanesulfonate (D151)

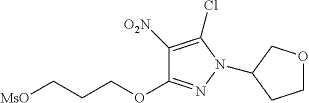 [SMILES: CS(=O)(=O)OCCCOc1nn(C2CCOC2)c(Cl)c1[N+](=O)[O-]]

To a solution of D150 (734 mg, 2.52 mmol) in CH₂Cl₂ (20 ml) and Et₃N (510 mg, 5.04 mmol) was added MsCl (432 mg, 3.77 mmol) at 0° C. The reaction was stirred at 0° C. for 0.5 h. The reaction was poured into ice water and extracted with CH₂Cl₂ (2×20 mL). The organic layer was washed with aq.NaHCO₃(30 mL), dried over anhydrous Na₂SO₄, filtered and concentrated to give the title compound as a white solid (870 mg, crude).

### Description D152

### 2-(3-((4-Nitro-5-chloro-1-(tetrahydrofuran-3-yl)-1H-pyrazol-3-yl)oxy)propyl) isoindoline-1,3-dione (D152)

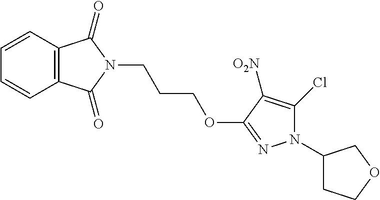 [SMILES: O=C1c2ccccc2C(=O)N1CCCOc1nn(C2CCOC2)c(Cl)c1[N+](=O)[O-]]

A mixture of D151 (870 mg, 2.35 mmol), potassium 1,3-dioxoisoindolin-2-ide (872 mg, 4.71 mmol), K₂CO₃ (325 mg, 2.35 mmol) and TBAI (89 mg, 0.24 mmol) in CH₃CN/DMF (20 mL/10 mL) was stirred at 100° C. for 4 hrs. The reaction was poured into ice water (40 mL) and extracted with EtOAc (2×30 mL). The organic layer was washed with brine (3×30 mL), dried, filtered and concentrated to give the compound. The crude product was purified by column chromatography on silica gel (PE:EtOAc=1:1) to give the title compound as a yellow solid (756 mg, yield 72%).

### Description D153

### 2-(3-((4-Amino-5-chloro-1-(tetrahydrofuran-3-yl)-1H-pyrazol-3-yl)oxy)propyl) isoindoline-1,3-dione (D153)

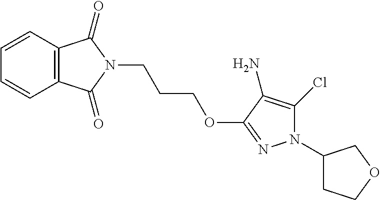 [SMILES: Nc1c(OCCCN2C(=O)c3ccccc3C2=O)nn(C2CCOC2)c1Cl]

To a solution of D152 (756 mg, 1.80 mmol) in conc.HCl (15 mL) was added SnCl₂ (681 mg, 3.59 mmol). The reaction was stirred at 60° C. for 20 min. The reaction was poured into ice and aq NaHCO₃(40 mL). The mixture extracted with EtOAc (2×50 mL). The organic layer was dried, filtered and concentrated to give the title compound as yellow oil (423 mg, yield 60%).

### Description D154

### 3-(3-Aminopropoxy)-5-chloro-1-(tetrahydrofuran-3-yl)-1H-pyrazol-4-amine (D154)

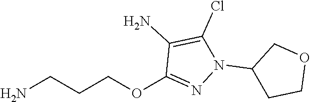

To a solution of D153 (430 mg, 1.10 mmol) in EtOH (10 mL) was added Hydrazine hydrate (0.5 mL). The reaction was stirred at 50° C. for 2 hrs. The mixture was poured into ice water (20 mL) and extracted with CH₂Cl₂(2×20 mL). The organic layer was dried, filtered and concentrated to give the title compound as yellow oil (314 mg, crude).

### Description D155

### N-(3-((4-Amino-5-chloro-1-(tetrahydrofuran-3-yl)-1H-pyrazol-3-yl)oxy)propyl)-2,5-dichloropyrimidin-4-amine (D155)

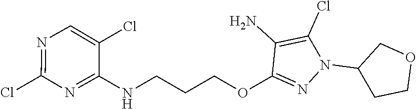

To a solution of D154 (283 mg, 1.09 mmol) in i-PrOH (15 mL) and Et₃N (221 mg, 2.18 mmol) were added a solution of 2,4,5-trichloropyrimidine (189 mg, 1.03 mmol) in i-PrOH (1 mL).

The reaction was stirred at room temperature for 0.5 hour. The reaction was poured into ice water (20 mL) and extracted with EtOAc (2×20 mL). The organic layer was dried, filtered and concentrated. The residue was purified by column chromatography on silica gel (PE:EtOAc=1:2) to give the title compound as a clear oil (280 mg, yield 63%).

### Description D156

### 5-Chloro-4-nitro-3-(3-((tetrahydro-2H-pyran-2-yl)oxy)propoxy)-1-(tetrahydro-2H-pyran-4-yl)-1H-pyrazole (D156)

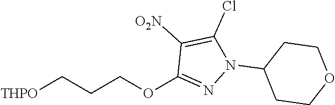

The title compound D156 was prepared using procedures similar to those used for preparing D149, starting from D36.

### Description D157

### 5-Cyclopropyl-4-nitro-3-(3-((tetrahydro-2H-pyran-2-yl)oxy)propoxy)-1-(tetrahydro-2H-pyran-4-yl)-1H-pyrazole (D157)

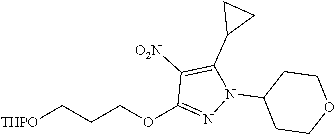

A mixture of D156 (832 mg, 2.13 mmol), tricyclohexyl phosphine (59 mg, 0.21 mmol), cyclopropylboronic acid (367 mg, 4.27 mmol), Pd(dppf)Cl₂ (154 mg, 0.21 mmol) and K₃PO₄ (907 mg, 4.27 mmol) in dioxane (20 mL) was stirred at 100° C. for 16 hrs. The reaction mixture was filtered and purified by column chromatography on silica gel (PE:EtOAc=2:1) to give the title compound as a yellow solid (431 mg, yield 51%).

Description for D158

### (R)-tert-Butyl 3-(3-(3-((2,5-dichloropyrimidin-4-yl)amino)butoxy)-5-methyl-4-nitro-1H-pyrazol-1-yl)azetidine-1-carboxylate (D158)

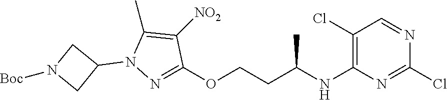 [SMILES: Cc1c([N+](=O)[O-])c(OCC[C@@H](C)Nc2nc(Cl)ncc2Cl)nn1C1CN(C(=O)OC(C)(C)C)C1]

To a solution of D79 (540 mg, 1.50 mmol) and K₂CO₃ (622 mg, 4.5 mmol) in DMSO (10 mL) was added tert-butyl 3-iodoazetidine-1-carboxylate (637 mg, 2.25 mmol). The reaction was stirred at 75° C. for 2 hrs. The mixture was cooled to room temperature was extracted with EtOAc and water. The organic layer was dried over anhydrous Na₂SO₄ and concentrated. The crude was purified by pre-TLC (PE:EA=1:1) to give the title compound as colorless oil (490 mg, yield 63.4%). ¹H NMR (400 MHz, CDCl₃): δ 7.96 (s, 1H), 5.67 (d, J=8.0 Hz, 1H), 4.97˜4.93 (m, 1H), 4.59˜4.55 (m, 1H), 4.49 (t, 2H), 4.42˜4.38 (m, 1H), 4.33˜4.25 (m, 3H), 4.17˜4.09 (m, 2H), 3.82˜3.78 (m, 1H), 2.58 (s, 3H), 2.24˜2.19 (m, 1H), 2.15˜2.07 (m, 1H), 1.62 (s, 2H), 1.46 (s, 9H), 1.44 (s, 3H), 1.38 (d, J=6.4 Hz, 2H).

### Description D159

### tert-Butyl 3-[(11R)-14-chloro-4,11-dimethyl-8-oxa-2,5,6,12,16,17-hexaazatricyclo [11.3.1.0^{3,7}]heptadeca-1(16),3,6,13(17),14-pentaen-5-yl]azetidine-1-carboxylate (D159)

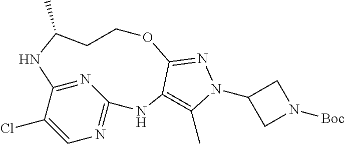 [SMILES: Cc1c2c(nn1C1CN(C(=O)OC(C)(C)C)C1)OCC[C@@H](C)Nc1nc(ncc1Cl)N2]

To a solution of D158 (380 mg, 0.74 mmol) in AcOH/MeOH (11.4 mL/114 mL) was added Zn (482 mg, 7.38 mmol). The mixture was stirred under argon at 75° C. for 3 hrs. The mixture was evaporated to dryness and the crude was diluted with PE:EtOAc=10:1 and the solid was filtrated to give the title compound as a rufous solid (1.4 g, yield 100%, contained some ZnOAc).

### Description D160

### (11R)-5-(Azetidin-3-yl)-14-chloro-4,11-dimethyl-8-oxa-2,5,6,12,16,17-hexaazatricyclo [11.3.1.0^{3,7}]heptadeca-1(16),3,6,13(17),14-pentaene, HCl salt (D160)

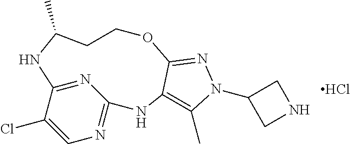 [SMILES: Cc1c2c(nn1C1CNC1)OCC[C@@H](C)Nc1nc(ncc1Cl)N2 ; Cl]

D159 (1.35 g, 0.7 mmol) was dissolved in HCl/MeOH (3 M, 20 mL) and the reaction was stirred at room temperature for 1.5 hrs. The mixture was the concentrated and the crude was used to next step directly without purification.

### Description D161

### 2,5-Dichloro-N-(4-((1-(2,2-diethoxyethyl)-5-methyl-4-nitro-1H-pyrazol-3-yl)oxy)butan-2-yl)pyrimidin-4-amine (D161)

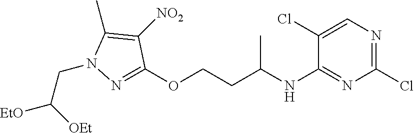 [SMILES: CCOC(Cn1nc(OCCC(C)Nc2nc(Cl)ncc2Cl)c([N+](=O)[O-])c1C)OCC]

To a solution of D73 (1.0 g, 2.7 mmol) in DMSO (50 mL) were added K₂CO₃ (1.2 g, 8.3 mmol) and 2-bromo-1,1-diethoxyethane (2.7 g, 13.8 mmol). The reaction was stirred at 90° C. for 4 hrs. The mixture was poured into ice water and extracted with EtOAc (3×100 mL). The combined organic layer was washed with brine, dried over anhydrous Na₂SO₄ and concentrated to give the title compound as an off-white solid, which was used in next step without further purification (960 mg).

### Description D162

### 14-Chloro-5-(2,2-diethoxyethyl)-4,11-dimethyl-8-oxa-2,5,6,12,16,17-hexaazatricyclo [11.3.1.0^{3,7}]heptadeca-1(16),3,6,13(17),14-pentaene (D162)

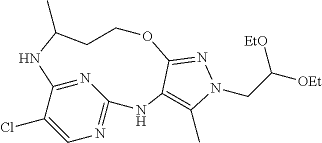 [SMILES: CCOC(Cn1nc2c(c1C)Nc1ncc(Cl)c(n1)NC(C)CCO2)OCC]

To a solution of D161 (960 mg, 2.0 mmol) in EtOH (50 mL) was added Fe (560 mg, 10 mmol) and a solution of NH₄Cl (535 mg, 10 mmol) in H₂O (5 mL). The reaction was stirred at 90° C. overnight. The mixture was filtered, the filter cake was washed with CH₂Cl₂:MeOH (10:1) (3×100 mL) and the combined filtrate was concentrated. The residue was poured into the sat.NaHCO₃ (aq) and extracted with CH₂Cl₂ (3×100 mL). The combined organic layer was washed with brine, dried over anhydrous Na₂SO₄ and concentrated. The crude was slurry in MeCN to give the title compound, which was directly used in next step (770 mg).

### Description D163

### 2-{14-Chloro-4,11-dimethyl-8-oxa-2,5,6,12,16,17-hexaazatricyclo[11.3.1.0^{3,7}]heptadeca-1(16),3,6,13(17),14-pentaen-5-yl}acetaldehyde, TFA salt (D163)

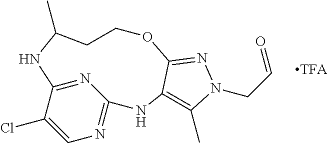 [SMILES: Cc1c2c(nn1CC=O)OCCC(C)Nc1nc(ncc1Cl)N2 ; "·TFA"]

To a solution of D162 (770 mg) in DCM (15 mL) was added TFA (15 mL) at room temperature. The reaction was stirred at room temperature overnight. The mixture was concentrated to give the title compound, which was directly used in next step (810 mg).

### Description D164

### 4-(3-(3-((2,5-Dichloropyrimidin-4-yl)amino)propoxy)-5-methyl-4-nitro-1H-pyrazol-1-yl)tetrahydro-2H-pyran-3-ol (D164)

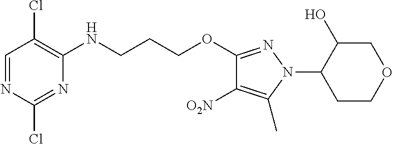 [SMILES: Cc1c([N+](=O)[O-])c(OCCCNc2nc(Cl)ncc2Cl)nn1C1CCOCC1O]

To a solution of D33 (800 mg, 2.30 mmol) in DMSO (12 mL) in a sealable vessel were added K₂CO₃ (1.27 g, 9.19 mmol) and 3,7-dioxabicyclo[4.1.0]heptane (1.84 g, 18.4 mmol). The vessel was sealed and the reaction was stirred at 95° C. overnight. The cooled reaction mixture was poured into water (120 mL) and the resultant was extracted with EtOAc (3×80 ml). The combined organics were washed with brine (50 mL), dried over anhydrous Na₂SO₄ and filtered. The filtrate was concentrated and the residue was purified by silica gel chromatography (CH₂Cl₂:EtOAc=5:1˜2:1) to give the title compound as an off-white solid (266 mg, yield 25%). LC-MS: 447.2 [M+H]⁺.

### Description D165

### 4-(3-(3-((2,5-Dichloropyrimidin-4-yl)amino)propoxy)-5-methyl-4-nitro-1H-pyrazol-1-yl)dihydro-2H-pyran-3(4H)-one (D165)

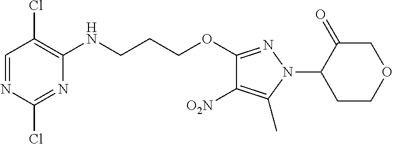 [SMILES: Cc1c([N+](=O)[O-])c(OCCCNc2nc(Cl)ncc2Cl)nn1C1CCOCC1=O]

To a stirred solution of D164 (266 mg, 0.595 mmol) in DMSO (4.0 mL) under argon at room temperature was added IBX (250 mg, 0.893 mmol). The reaction was stirred at 55° C. overnight. The reaction mixture poured into water (100 mL) and extracted with EtOAc (3×60 ml). The combined organics were washed with brine (50 mL), dried over anhydrous Na₂SO₄, filtered. The filtrate was concentrated and the crude was purified by chromatography on silica gel (CH₂Cl₂:EtOAc=5:1˜3:1) to give the title compound as an off-white solid (115 mg, yield 43%). LC-MS: 445.2 [M+H]⁺.

### Description D166

### 2,5-Dichloro-N-(3-((1-(3,3-difluorotetrahydro-2H-pyran-4-yl)-5-methyl-4-nitro-1H-pyrazol-3-yl)oxy)propyl)pyrimidin-4-amine (D166)

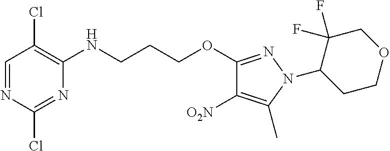 [SMILES: Cc1c([N+](=O)[O-])c(OCCCNc2nc(Cl)ncc2Cl)nn1C1CCOCC1(F)F]

To a suspension of D165 (115 mg, 0.258 mmol) in CH₂Cl₂ (15 mL) under argon at 0° C. was added DAST (208 mg, 1.29 mmol) via syringe. The reaction was allowed to warm gradually to room temperature and stirred over 2 days. The mixture was poured into sat. NaHCO₃(30 mL) and extracted with CH₂Cl₂ (3×30 mL). The combined organics were washed with brine (50 mL), dried over anhydrous Na₂SO₄, filtered. The filtrate was concentrated and the crude was purified by chromatography on silica gel (CH₂Cl₂:EtOAc=5:1) to give the title compound as an off-white solid (103 mg, yield 85%). LC-MS: 467.2 [M+H]⁺.

### Description D167

### 5-(3-Bromo-5-methyl-4-nitro-1H-pyrazol-1-yl)dihydro-2H-pyran-3(4H)-one (D167)

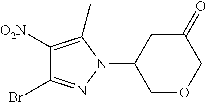 [SMILES: Cc1c([N+](=O)[O-])c(Br)nn1C1COCC(=O)C1]

To a stirred suspension of 5-bromo-3-methyl-4-nitro-1H-pyrazole_(1.85 g, 8.98 mmol) in dry CH₂Cl₂ (20 mL) under argon at room temperature were added 2H-pyran-3(6H)-one (1.77 g, 18.0 mmol) and ScCl₃ (123 mg, 0.813 mmol). The reaction was then stirred under argon at 25° C. overnight. The reaction mixture was concentrated to a volume of c. 10 mL and the resulting yellow suspension was directly loaded onto a silica gel column (PE:EtOAc=5:1˜1:1) to give the title compound as off-white solid (1.98 g, yield 72%). LC-MS: 306.2 [M+H]⁺. ¹H NMR (400 MHz, CDCl₃): δ 4.88˜4.81 (m, 1H), 4.21˜4.10 (m, 3H), 4.03 (dd, J=11.6, 8.0 Hz, 1H), 3.24 (dd, J=16.4, 8.6 Hz, 1H), 2.95 (dd, J=16.4, 6.0 Hz, 1H), 2.71 (s, 3H).

### Description D168

### 5-(3-Bromo-5-methyl-4-nitro-1H-pyrazol-1-yl)tetrahydro-2H-pyran-3-ol (D168)

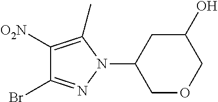 [SMILES: Cc1c([N+](=O)[O-])c(Br)nn1C1COCC(O)C1]

To a stirred suspension of D167 (1.98 g, 6.51 mmol) in MeOH/THF (30 mL/30 mL) under argon at 0° C. was added NaBH₄ (124 mg, 3.28 mmol) in one portion. The reaction was stirred under argon at 0° C. for 1 hour. The reaction was quenched by addition of saturated aqueous NH₄Cl (50 mL) at 0° C. The resultant was diluted with water (20 mL) and extracted with EtOAc (3×50 mL). The combined organics were washed with brine (50 mL), dried over anhydrous Na₂SO₄ and filtered. The filtrate was concentrated and the residue was dried under vacuo afforded crude desired product as light yellow solid (2.07 g, crude). LC-MS: 308.2 [M+H]⁺.

### Description D169

### 1-(5-Hydroxytetrahydro-2H-pyran-3-yl)-5-methyl-4-nitro-1H-pyrazol-3-ol (D169)

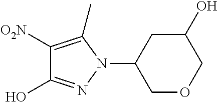 [SMILES: Cc1c([N+](=O)[O-])c(O)nn1C1COCC(O)C1]

To a suspension of D168 (2.07 g, 6.51 mmol) in water (50 mL) was added KOH (5.0 g, 89.1 mmol). The reaction was stirred under argon at 105° C. for 3 hrs. The cooled mixture was extracted with Et₂O (2×50 mL). The aqueous layer was acidified with conc. HCl until pH=2. The mixture was extracted with EtOAc (3×50 mL) and the combined organics were washed with brine (30 mL), dried over anhydrous Na₂SO₄, filtered and concentrated. The crude was suspended in CH₂Cl₂ (10 mL), stirred for 15 minutes, filtered. The filter cake was dried under infrared lamp and collected to give the title compound as a yellow solid (1.01 g, yield over 2 steps 63%). LC-MS: 244.3 [M+H]⁺. ¹H NMR (400 MHz, DMSO-d₆): δ 11.41 (s, 1H), 5.11 (d, J=5.2 Hz, 1H), 4.42˜4.36 (m, 1H), 3.89˜3.81 (m, 2H), 3.72˜3.66 (m, 1H), 2.93 (t, J=10.4 Hz, 1H), 2.58 (s, 3H), 2.22˜2.19 (m, 1H), 1.85 (q, J=11.6 Hz, 1H).

### Description D170

### tert-Butyl (3-((1-(5-hydroxytetrahydro-2H-pyran-3-yl)-5-methyl-4-nitro-1H-pyrazol-3-yl)oxy)propyl)carbamate (D170)

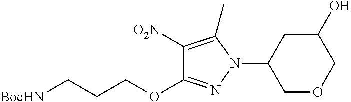 [SMILES: Cc1c([N+](=O)[O-])c(OCCCNC(=O)OC(C)(C)C)nn1C1COCC(O)C1]

To a solution of D169 (400 mg, 1.64 mmol) in DMF (5.0 mL) under argon at room temperature were added K₂CO₃ (680 mg, 4.92 mmol) and tert-butyl (3-bromopropyl) carbamate (587 mg, 2.47 mmol). The reaction was stirred under argon at 85° C. for 3 hrs. The cooled reaction mixture was poured into water (60 mL) and the resultant was extracted with EtOAc (3×40 mL). The combined organics were washed with brine (30 mL), dried over anhydrous Na₂SO₄, filtered and concentrated. The crude was purified by chromatography on silica gel (PE:EtOAc=5:1˜1:1) to give the title compound as light yellow oil (331 mg, yield 50%). LC-MS: 401.3 [M+H]⁺.

### Description D171

### tert-Butyl (3-((1-(5-fluorotetrahydro-2H-pyran-3-yl)-5-methyl-4-nitro-1H-pyrazol-3-yl)oxy) propyl)carbamate (D171)

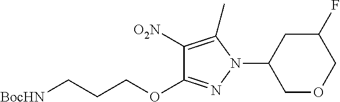 [SMILES: Cc1c([N+](=O)[O-])c(OCCCNC(=O)OC(C)(C)C)nn1C1COCC(F)C1]

To a solution of D170 (331 mg, 0.827 mmol) in CH₂Cl₂ (10 mL) under argon at 0° C. was added DAST (660 mg, 4.09 mmol) via syringe. The reaction was stirred under argon at room temperature overnight. The mixture was diluted with CH₂Cl₂ (20 mL) and sat. NaHCO₃(30 mL). The resultant was stirred at room temperature for 1 hour and the organic layer was separated. The aqueous layer was extracted with CH₂Cl₂ (2×30 mL) and the combined organics were washed with brine (30 mL), dried over anhydrous Na₂SO₄, filtered and concentrated. The crude was purified by chromatography on silica gel (PE:EtOAc=5:1˜2:1) to give the title compound as light yellow oil (338 mg). LC-MS: 425.3 [M+H]⁺.

### Description D172

### (R)-2,5-Dichloro-N-(4-((5-methyl-4-nitro-1-(1,4-dioxaspiro[4.5]decan-8-yl)-1H-pyrazol-3-yl)oxy)butan-2-yl)pyrimidin-4-amine (D172)

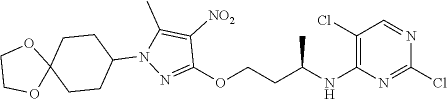 [SMILES: Cc1c([N+](=O)[O-])c(OCC[C@@H](C)Nc2nc(Cl)ncc2Cl)nn1C1CCC2(CC1)OCCO2]

A solution of D79 (2.5 g, 6.9 mmol), D82 (8.1 g, 34.3 mmol) and K₂CO₃ (2.8 g, 37.5 mmol) in DMA (50 mL) was heated to 80° C. and stirred for 16 hrs. The mixture was poured into water (300 mL) and extracted with EtOAc (3×100 mL). The combined organic layer was washed with brine, dried over anhydrous Na₂SO₄ and concentrated. The crude was purified by column chromatography on silica gel (PE:EtOAc=5:1˜1:1) to give the title compound as little yellow oil (1.3 g, yield 37%). LC-MS: 501.1 [M+H]⁺.

### Description D173

### (R)-4-(3-(3-((2,5-dichloropyrimidin-4-yl)amino)butoxy)-5-methyl-4-nitro-1H-pyrazol-1-yl)cyclohexanone (200 mg, 0.44 mmol) (D173)

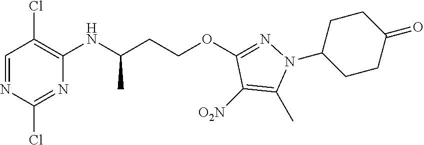 [SMILES: Cc1c([N+](=O)[O-])c(OCC[C@@H](C)Nc2nc(Cl)ncc2Cl)nn1C1CCC(=O)CC1]

To a solution of D172 (1.3 g, 2.85 mmol) in DCM (25 mL) was TFA (5 mL) and the reaction was stirred at 60° C. for 5 hrs. The cooled mixture was added water (100 mL) and sat. NaHCO₃ until pH>7. The organic layer was washed with sat. NaHCO₃, brine, dried over Na₂SO₄, and concentrated. The product was purified by column chromatography on silica gel (PE:EA=5:1˜1:1) to give the title compound (800 mg, yield 67%).

### Description D174

### (R)-2,5-dichloro-N-(4-((1-(4,4-difluorocyclohexyl)-5-methyl-4-nitro-1H-pyrazol-3-yl)oxy)butan-2-yl)pyrimidin-4-amine (D174)

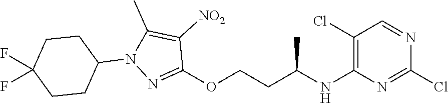

To a solution of D173 (200 mg, 0.44 mmol) in DCM (20 mL) was added DAST (211 mg, 1.32 mmol) dropwise at −60° C. under nitrogen. The reaction was stirred at room temperature for 16 hrs. The mixture was diluted with MeOH (5 mL), stirred for 10 min and then concentrated. The crude was purified by column chromatography on silica gel (PE:EA=10:1˜1:1) to give the title compound (60 mg, yield 28%). LC-MS: 479.2 [M+H]⁺.

### Description D175

### (R)-4-(3-(3-((2,5-Dichloropyrimidin-4-yl)amino)butoxy)-5-methyl-4-nitro-1H-pyrazol-1-yl)cyclohexanol (D175)

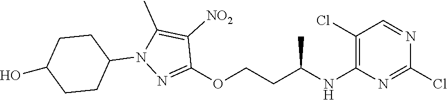

To a solution of D173 (760 mg, 1.66 mmol) in MeOH (20 mL) were added NaBH₄ (75 mg, 1.97 mmol) at 5˜10° C. The reaction was stirred at room temperature for 4 hrs. The mixture was poured into water (100 mL) and extracted with EtOAc (2×100 mL). The organic layer was washed with brine, dried over anhydrous Na₂SO₄ and concentrated. The crude was purified by column chromatography on silica gel (PE:EA=5:1) to give the title compound (410 mg, yield 53%). LC-MS: 459.2 [M+H]⁺.

### Description D176

### (R)-2,5-Dichloro-N-(4-((1-(4-(difluoromethoxy)cyclohexyl)-5-methyl-4-nitro-1H-pyrazol-3-yl)oxy)butan-2-yl)pyrimidin-4-amine (D176)

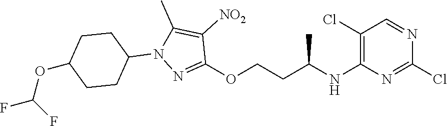

To a solution of D175 (250 mg, 0.54 mmol) in CH₃CN (20 mL) was added CuI (20.5 mg, 0.108 mmol). After stirred at 50° C. for 10 min, 2,2-difluoro-2-(fluorosulfonyl)acetic acid (146 mg, 0.82 mmol) in MeCN (0.5 mL) was added drop wise. The reaction was stirred at 50° C. for 2 hrs. The mixture was concentrated and the crude was purified by column chromatography on silica gel (PE:EtOAc=3:1) to give the title compound (110 mg, crude). LC-MS: 509.2 [M+H]⁺.

### Description D177

### (R)-3-(3-(3-((2,5-Dichloropyrimidin-4-yl)amino)butoxy)-5-methyl-4-nitro-1H-pyrazol-1-yl)cyclobutanol (D177)

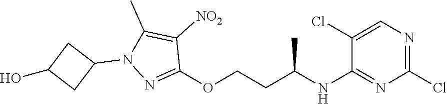

To a solution of D134 (770 mg, 1.8 mmol) in MeOH (20 mL) was added NaBH₄ (102 mg, 2.6 mmol) at 5˜10° C. The reaction was stirred at room temperature for 4 hrs. The mixture was concentrated and the crude was purified by column chromatography on silica gel (PE:EtOAc=2:1) to give the title compound as a white solid (700 mg, yield 95%). LC-MS: 431.1 [M+H]⁺.

### Description D178

### 3-(3-(3-((2,5-Dichloropyrimidin-4-yl)(methyl)amino)butoxy)-5-methyl-4-nitro-1H-pyrazol-1-yl)cyclobutanol (D178)

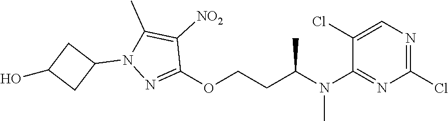

To a solution of D177 (215 mg, 0.5 mmol) in DMF (20 mL) were added CH₃I (85.2 mg, 0.6 mmol) and NaH (0.39 g, 9.9 mmol). The reaction was stirred at room temperature for 5 hrs. The mixture was poured into water (50 mL) and extracted with EtOAc (2×100 mL). The combined organic layer was washed with brine, dried over anhydrous Na₂SO₄ and concentrated in vacuo. The crude was purified by column chromatography on silica gel (PE:EtOAc=1:1) to give the title compound as yellow oil (100 mg). LC-MS: 445.2 [M+H]⁺.

### Description D179

### Tert-butyl 7-oxa-3-azabicyclo[4.1.0]heptane-3-carboxylate (D179)

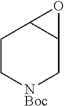 [SMILES: CC(C)(C)OC(=O)N1CCC2OC2C1]

To a solution of tert-butyl 3,6-dihydropyridine-1(2H)-carboxylate (18.3 g, 1.0 mol) and m-CPBA (34.5 g, 2.0 mol) in CH₂Cl₂ (200 mL) were stirred at room temperature overnight. The reaction mixture was filtered and extracted with CH₂Cl₂ (300 mL). The combined organic layer was washed with brine, dried over Na₂SO₄ and concentrated in vacuo to give the target product (17.7 g, yield 89%). The crude product was used directly for the next step.

### Description D180

### Tert-butyl 4-(3-((R)-3-((2,5-dichloropyrimidin-4-yl)amino)butoxy)-5-methyl-4-nitro-1H-pyrazol-1-yl)-3-hydroxypiperidine-1-carboxylate (D180)

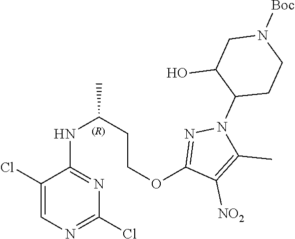

To a solution of D79 (4.8 g, 13.1 mmol), D179 (13 g, 65.5 mmol) and K₂CO₃ (5.5 g, 39.3 mmol) in DMSO (50 mL) was stirred at 90° C. overnight. The reaction mixture was poured into ice-water and extracted with EtOAc (3×50 mL). The combined organic layer was washed with brine, dried over Na₂SO₄ and concentrated in vacuo. The crude product was purified by column chromatography on silica gel (PE:EtOAc=1:1) to give the target product as a light yellow solid (1.42 g, yield 19%).

### Description D181

### Tert-butyl 4-(3-((R)-3-((2,5-dichloropyrimidin-4-yl)amino)butoxy)-5-methyl-4-nitro-1H-pyrazol-1-yl)-3-oxopiperidine-1-carboxylate (D181)

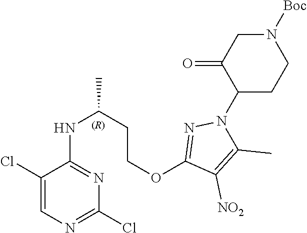

To a solution of D180 (1.42 g, 2.53 mmol) and Dess-Martin periodinane (2.15 g, 5.06 mmol) in CH₂Cl₂ (30 mL) were stirred at room temperature for 2 hrs. The reaction mixture was extracted with CH₂Cl₂ (3×50 mL). The combined organic layer was washed with brine, dried over with Na₂SO₄ and concentrated in vacuo to give the target product (1.33 g crude). The crude product was used directly for the next step.

### Description D182

### Tert-butyl 4-(3-((R)-3-((2,5-dichloropyrimidin-4-yl)amino)butoxy)-5-methyl-4-nitro-1H-pyrazol-1-yl)-3,3-difluoropiperidine-1-carboxylate (D182)

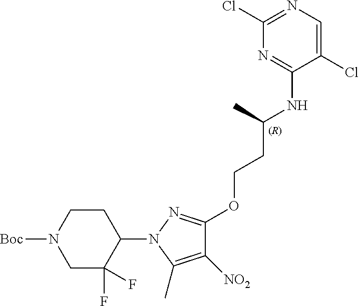

To a solution of D181 (1.42 g, 2.53 mmol) in CH₂Cl₂ (50 mL) was added DAST (816 mg, 5.06 mmol) drop wise at −78° C. The mixture was stirred at room temperature for 3 hrs. The reaction mixture was poured into ice-water and extracted with CH₂Cl₂ (3×50 mL). The combined organic layer was washed with brine, dried over with Na₂SO₄ and concentrated in vacuo to give the target product (1.5 g crude). The crude product was used directly for the next step.

### Description D183

### 3-Methyltetrahydro-2H-pyran-4-ol (D183)

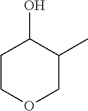

To a solution of 3-methyldihydro-2H-pyran-4(3H)-one (1.0 g, 8.9 mmol) in MeOH (20 mL) was added slowly NaBH₄ (410 mg, 10.7 mmol) at 0˜5° C. The reaction solution was stirred at the same temperature for 1 hr. Water (3 mL) was added with dropwise for 10 min. The reaction solution was concentrated in vacuo to give residue. CH₂Cl₂ (15 mL) was added and filtered, the filtrate was concentrated to afford the title compound (780 mg, 76% yield) as a colorless oil.

### Description D184

### 3,4,5-Tribromo-1-(3-methyltetrahydro-2H-pyran-4-yl)-1H-pyrazole (D184)

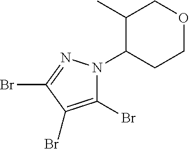

To a solution of D183 (0.95 g, 8.3 mmol) in THF (50 mL) was added 3,4,5-tribromo-1H-pyrazole (2.8 g, 9.1 mmol), PPh3 (4.3 g, 16.6 mmol) and DEAD (3.6 g, 20.7 mmol) at 0˜5° C. under N₂. The reaction solution was stirred at rt for 16 hrs. The reaction solution was poured into water (50 mL) and extracted with EtOAc (100 mL×2). The combined organic layer was washed with brine, dried over anhydrous Na₂SO₄ and concentrated in vacuo. The crude product was purified by gel silica column chromatography (PE:EtOAc from 50:1 to 10:1) to afford the title compound as yellow oil (2.4 g, yield 71.6%). LC-MS 403.0 (M+H)⁺.

### Description D185

### 3,4-Dibromo-5-methyl-1-(3-methyltetrahydro-2H-pyran-4-yl)-1H-pyrazole (D185)

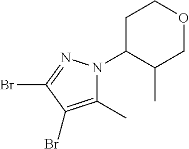

To a solution of D184 (2.4 g, 6.0 mmol) in THF (50 mL) was added n-BuLi (4.1 mL, 6.6 mmol) with dropwise at −50° C. under N2. The mixture solution was stirred at −60° C. for 1 hr. CH₃I (1.8 g, 12.66 mmol) was added with dropwise. The reaction solution was stirred at the same temperature for another 2 hrs. Water (10 mL) was added and extracted with EtOAc (100 mL×2). The combined organic layer was washed with brine, dried over anhydrous Na₂SO₄ and concentrated in vacuo. The crude product was purified by gel silica column chromatography (PE:EtOAc from 20:1 to 10:1) to afford title compound as colorless oil (1.5 g, yield 89%). LC-MS 339.0 (M+H)⁺.

### Description D186

### 3-Bromo-5-methyl-1-(3-methyltetrahydro-2H-pyran-4-yl)-4-nitro-1H-pyrazole (D186)

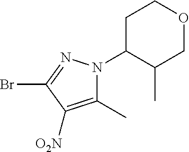 [SMILES: Cc1c([N+](=O)[O-])c(Br)nn1C1CCOCC1C]

To a solution of H₂SO₄ (10 mL) and HNO₃ (5 mL) was added D185 (2.6 g, 7.7 mmol) at 0−10° C. The reaction was stirred at 0° C. for 1 hr. The reaction solution was poured into ice-water (100 mL) and extracted with EtOAc (50 mL×3). The combined organic layer was washed with NaHCO₃ aqueous solution, brine, dried over anhydrous Na₂SO₄ and concentrated in vacuo. The crude product was purified by gel silica column chromatography (PE:EtOAc from 10:1 to 5:1) to afford title compound as a little yellow oil (1.0 g, yield 42%). LC-MS 304.1 (M+H)⁺.

### Description D187

### 5-Methyl-1-(3-methyltetrahydro-2H-pyran-4-yl)-4-nitro-1H-pyrazol-3-ol (D187)

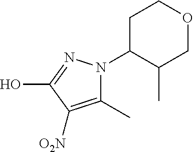 [SMILES: Cc1c([N+](=O)[O-])c(O)nn1C1CCOCC1C]

To a solution of D186 (1.0 g, 3.2 mmol) in H₂O (25 mL) was added KOH (3.7 g, 65.5 mmol)). The reaction was heated to 120° C. for 12 hrs. The reaction was cooled to room temperature and extracted with EtOAc (15 mL). The aqueous phase was neutralized with 2N HCl to pH=4˜5 and extracted with EtOAc (25×4 mL). The combined organic layers were dried over anhydrous Na₂SO₄, filtered and concentrated in vacuo to afford title compound as yellow oil (450 mg, yield 57%). LC-MS 242.3 (M+H)⁺.

### Description D188

### Tert-butyl (R)-(4-((4-nitro-1-(tetrahydro-2H-pyran-4-yl)-1H-pyrazol-3-yl)oxy)butan-2-yl)carbamate (D188)

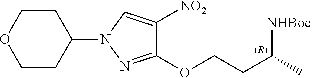

To a solution of D36, (R)-3-((tert-butoxycarbonyl)amino)butyl methanesulfonate (2.7 g, 10.1 mmol) and K₂CO₃ (2.3 g, 16.8 mmol) in DMF (100 mL) were stirred at 100° C. for 2 hrs. The reaction mixture was poured into ice-water and extracted with EtOAc (3×200 mL). The combined organic layer was washed with brine, dried over Na₂SO₄ and concentrated in vacuo. The crude product was purified by column chromatography on silica gel (PE:EtOAc=3:1) to give the target product as an off-white solid. (1.4 g, yield 45%).

### Description D189

### Tert-butyl (R)-(4-((5-iodo-4-nitro-1-(tetrahydro-2H-pyran-4-yl)-1H-pyrazol-3-yl)oxy)butan-2-yl)carbamate (D189)

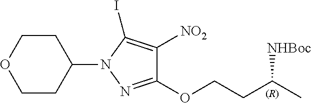

To a solution of D188 (1.4 g, 3.6 mmol) in THF (50 mL) was added LiHMDS (10.8 mL, 1 M, 10.8 mmol) at −60° C. under N2. After it was stirred at −60° C. under N2 for 1 hr, I₂ (1.1 g, 4.32 mmol) in THF (5 mL) was added at −60° C. The mixture was stirred at −60° C. for 2 hrs. The reaction mixture was poured into the sat.NH₄Cl (aq) and extracted with EtOAc (3×200 mL). The combined organic layer was washed with brine, dried over Na₂SO₄ and concentrated in vacuo. The crude product was purified by column chromatography on silica gel (PE:EtOAc=5:1) to give the target product as an off-white solid (1.7 g, yield 85%).

### Description D190

### Tert-butyl (R)-(4-((4-nitro-1-(tetrahydro-2H-pyran-4-yl)-5-(trifluoromethyl)-1H-pyrazol-3-yl)oxy)butan-2-yl)carbamate (D190)

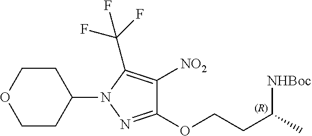

To a mixture of D189 (1.7 g, 3.33 mmol), methyl 2,2-difluoro-2-(fluorosulfonyl)acetate (2.56 g, 13.32 mmol) and CuI (1.3 g, 6.66 mmol) in DMF (50 mL) was stirred at 80° C. for 6 hrs under N2. The reaction mixture was poured into the sat.NaCl (aq) and extracted with EtOAc (3×200 mL). The combined organic layer was washed with brine, dried over Na₂SO₄ and concentrated in vacuo. The crude product was purified by column chromatography on silica gel to give the target product as an off-white solid. (1 g, yield 62.5%).

### Description D191

### 2-(2-Methyl-1,3-dioxolan-2yl)acetohydrazide (D191)

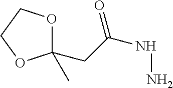

To a solution of ethyl 2-(2-methyl-1, 3-dioxolan-2yl)acetate (20.0 g, 114.8 mmol) in EtOH (100 mL) was added hydrazine (11.5 g, 229.6 mmol) at room temperature. The mixture was stirred at 80° C. overnight. The mixture was concentrated in vacuum to give yellow oil (20.5 g). ¹H NMR (400 MHz, CDCl₃): δ 7.68 (s, 1H), 4.00˜3.97 (m, 4H), 3.48 (s, 1H), 2.61 (s, 2H), 1.41 (s, 3H).

### Description D192

### N′-(3,3-Dimethyltetrahydro-2H-pyran-4-yl)-2-(2-methyl-1,3-dioxolan-2-yl)acetohydrazide (D192)

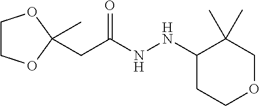 [SMILES: CC1(CC(=O)NNC2CCOCC2(C)C)OCCO1]

To a solution of 3, 3-dimethyldihydro-2H-pyran-4(3H)-one (1.0 g, 7.81 mmol) in MeOH (5 mL) was added D191 (1.25 g, 7.81 mmol) at room temperature. The mixture was stirred at room temperature for 1 hr. NaBH₃CN (1.48 g, 23.4 mmol) was added into the mixture and the mixture was stirred at room temperature for 1 hr. The mixture was filtered and washed with solution of NH₄Cl, dried over Na₂SO₄. The organic layer was concentrated in vacuum to give yellow oil (960 mg, yield 45%).

### Description D193

### 1-(3,3-Dimethyltetrahydro-2H-pyran-4-yl)-5-methyl-1H-pyrazol-3-ol (D193)

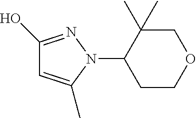

To a solution of D192 (1.80 g, 6.61 mmol) in EtOH (10 mL) was added TFA (1.51 g, 13.2 mmol) at room temperature. The mixture was stirred at 90° C. overnight. The mixture was concentrated and purified by column chromatography on silica gel (PE:EtOAc from 10:1 to 2:1) to give the target product as a yellow oil (1.18 g, yield 85%).

### Description D194

### 1-(3, 3-Dimethyltetrahydro-2H-pyran-4-yl)-5-methyl-4-nitro-1H-pyrazol-3-ol (D194)

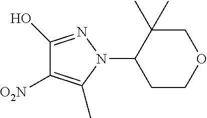 [SMILES: Cc1c([N+](=O)[O-])c(O)nn1C1CCOCC1(C)C]

To a solution of D193 (500 mg, 2.38 mmol) in H₂SO₄ (5 mL) was added KNO₃ (720 mg, 7.14 mmol) slowly at 0° C. The mixture was stirred at 0° C. for 30 minutes. The reaction mixture was poured into ice water slowly, and extracted with EtOAc (2×30 mL). The combined organic layer was washed with brine and dried over Na₂SO₄, concentrated in vacuum to give yellow solid (450 mg, yield 74%).

### Description D195

### 2-(3-Hydroxypropyl)isoindoline-1,3-dione (D195)

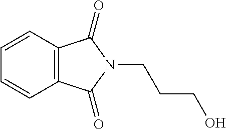 [SMILES: O=C1c2ccccc2C(=O)N1CCCO]

To a solution of isobenzofuran-1,3-dione (50 g, 0.337 mol) in toluene (300 mL) was added 3-aminopropan-1-ol (25.3 g, 0.377 mol). The mixture was stirred at 120° C. overnight. The mixture was concentrated and EA was added to the residue. The mixture was poured into NaOH (1M, 200 mL), stirred for 10 min, extracted with EA, washed with water and brine, dried over Na₂SO₄ and evaporated to give 2-(3-hydroxypropyl)isoindoline-1,3-dione as a white solid. (41 g, yield: 55%). ¹H NMR (400 MHz, CDCl₃) δ 7.78 (dt, J=7.0, 3.5 Hz, 2H), 7.74˜7.61 (m, 2H), 3.86˜3.73 (m, 2H), 3.55 (t, J=5.6 Hz, 2H), 2.41 (s, 1H), 1.82 (dt, J=12.1, 6.0 Hz, 2H).

### Description D196

### Ethyl 2-formyl-3,3-dimethylbutanoate (D196)

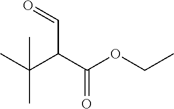

To a solution of ethyl 3,3-dimethylbutanoate (1.3 g, 1.0 mol) in dry THF (50 mL) was added LDA (7.5 mL) at −70° C. The mixture solution was stirred at −70° C. for 30 min. A solution of compound ethyl formate (2.2 g, 30 mmol) m in dry THF (10 mL) was added slowly. The mixture solution was stirred at −70° C. for 2 hrs and raise to room temperature overnight. The mixture was poured into NH₄Cl (sat.) and extracted with CH₂Cl₂ (50 mL×3). The combined organic layer was washed with brine, dried over Na₂SO₄, concentrated in vacuo. The crude product was used to next step without purification.

### Description D197

### 5-(tert-Butyl)-2-mercaptopyrimidin-4-ol (D197)

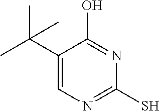

To a solution of D196 (1.7 g, crude) in dry H₂O (50 mL) was added thiourea (2.2 g, 30 mmol) at room temperature. The mixture solution was refluxed for 2 hrs. The reaction solution was cooled to rt and adjust PH=2 with HCl (6 M). The solid was collected and used in next step without purification. LC-MS: 185.2 (M+H)⁺. ¹HNMR (400 MHz, DMSO-d₆): δ 12.29 (s, 1H), 12.15 (s, 1H), 6.99 (d, J=5.6 Hz, 1H), 1.19 (s, 9H).

### Description D198

### 5-(tert-Butyl)pyrimidine-2,4-diol (D198)

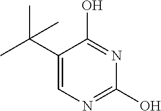

To a solution of D197 (1.7 g, 9.24 mmol) in HCl (50 mL, 6M) was added 2-chloroacetic acid (2.62 g, 27.7 mmol) at room temperature. The mixture solution was refluxed for 48 hrs. The reaction solution was cooled to rt, filtered and washed with water to give the title compound as a white solid. LC-MS: 169.2 (M+H)⁺. ¹HNMR (400 MHz, DMSO-d₆): δ 10.88 (s, 1H), 10.58 (s, 1H), 6.98 (d, J=5.0 Hz, 1H), 1.18 (s, 9H).

### Description D199

### 5-(tert-Butyl)-2,4-dichloropyrimidine (D199)

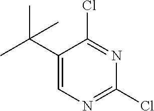

To a solution of D198 (1.1 g, 6.57 mmol) in toluene (50 mL) was added POCl₃ (3.0 g, 19.6 mmol) and DIEA (2.08 g, 16.35 mmol) at room temperature. The mixture solution was refluxed overnight. The reaction solution was poured into ice-water and extracted with EtOAc (50 ml×3). The combined organic layer was washed with NaHCO₃ (sat.), brine, dried over Na₂SO₄ and concentrated in vacuo. The crude product was purified by FCC (PE) to give the product as colorless oil. LC-MS: 205.2 (M+H)⁺. ¹HNMR (400 MHz, CDCl₃): δ 8.58 (s, 1H), 1.49 (s, 9H).

### Description D200

### 3-(Trifluoromethyl)dihydro-2H-pyran-4(3H)-one (D200)

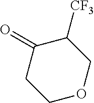 [SMILES: O=C1CCOCC1C(F)(F)F]

To a solution of dihydro-2H-pyran-4(3H)-one (1.0 g, 10.0 mmol) and pyrrolidine (0.71 g, 10.0 mmol) in THF (20 mL) was added DMAP (1.46 g, 12.0 mmol) at room temperature. The mixture was stirred at room temperature for 30 minutes. Then 5-(trifluoromethyl) dibenzothiophen-ium trifluoromethanesulfonate (4.97 g, 12.0 mmol) was added into the mixture. The mixture was stirred at room temperature overnight. TLC showed the reaction was completed. The mixture was concentrated in vacuum to give yellow oil. (500 mg, yield 29%)

### Description D201

### 2-(2-Methyl-1,3-dioxolan-2-yl)-N′-(3-(trifluoromethyl)tetrahydro-2H-pyran-4-yl)acetohydrazide (D201)

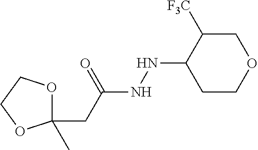 [SMILES: CC1(CC(=O)NNC2CCOCC2C(F)(F)F)OCCO1]

To a solution of D200 (500 mg, 2.98 mmol) in MeOH (40 mL) was added D191 (550 mg, 3.27 mmol) at room temperature. The mixture was stirred at room temperature overnight. NaBH₃CN (938.7 mg, 14.9 mmol) and AcOH (18.0 mg, 0.30 mmol) was added into the mixture and the mixture was stirred at room temperature for 1 h. The mixture was filtered and washed with solution of NH₄Cl and brine, extracted with EA (3×20 mL), and dried over Na₂SO₄. The solvent was concentrated in vacuum to give yellow oil. (220 mg, yield 23%)

### Description D202

### 5-Methyl-1-(3-(trifluoromethyl)tetrahydro-2H-pyran-4-yl)-1H-pyrazol-3-ol (D202)

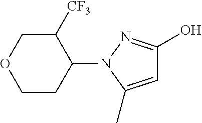 [SMILES: Cc1cc(O)nn1C1CCOCC1C(F)(F)F]

To a solution of D201 (220 mg, 0.70 mmol) in EtOH (5 mL) was added TFA (159.6 mg, 1.40 mmol) at room temperature. The mixture was stirred at 90° C. overnight. The mixture was concentrated and purified by column chromatography on silica gel (PE:EtOAc from 5:1 to 2:1) to give the target product as an yellow oil. (130 g, yield 74%).

### Description D203

### 5-Methyl-4-nitro-1-(3-(trifluoromethyl)tetrahydro-2H-pyran-4-yl)-1H-pyrazol-3-ol (D203)

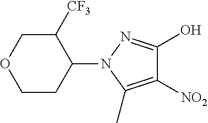 [SMILES: Cc1c([N+](=O)[O-])c(O)nn1C1CCOCC1C(F)(F)F]

To a solution of D202 (130 mg, 0.52 mmol) in H₂SO₄ (5 mL) was added KNO₃ (63 mg, 0.62 mmol) slowly at 0° C. The mixture was stirred at 0° C. for 30 minutes. The reaction mixture was poured into ice water slowly, and extracted with EtOAc (2×20 mL). The combined organic layer was washed with brine and dried over Na₂SO₄, concentrated in vacuum to give yellow solid. (140 mg, yield 90%)

### Example E1

### 14-Chloro-4-methyl-5-(oxan-4-yl)-8-oxa-2,5,6,12,16,17-hexaazatricyclo [11.3.1.0^{3,7}]heptadeca-1(16),3,6,13(17),14-pentaene (E1)

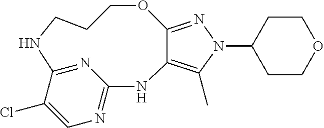 [SMILES: Cc1c2c(nn1C1CCOCC1)OCCCNc1nc(ncc1Cl)N2]

To a solution of D9 (121 mg, 0.30 mmol) in i-PrOH (5 mL) was added HCl (0.03 mL, 0.3 mmol). The reaction was stirred at 100° C. for 16 hrs. The reaction mixture was concentrated and the residue was diluted with EtOAc (20 mL) and washed with saturated NaHCO₃ aqueous solution (20 mL). Then the organic layer was concentrated and the residue was purified by prep-HPLC to give the title compound as a white solid (29 mg, yield 26.4%). LC-MS: 367.5[M+H]⁺. ¹H NMR (400 MHz, CDCl₃): δ 7.81 (s, 1H), 6.26 (s, 1H), 5.55 (s, 1H), 4.40 (br, 2H), 4.12˜4.10 (m, 3H), 3.48˜3.36 (m, 4H), 2.29˜2.21 (m, 5H), 1.92 (br, 2H), 1.80˜1.74 (m, 2H).

### Example E2

### 14-Chloro-10-methoxy-4-methyl-5-(oxan-4-yl)-8-oxa-2,5,6,12,16,17-hexaazatricyclo [11.3.1.0^{3,7}]heptadeca-1(16),3,6,13(17),14-pentaene (E2)

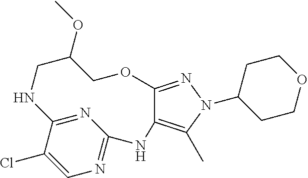 [SMILES: COC1CNc2nc(ncc2Cl)Nc2c(nn(C3CCOCC3)c2C)OC1]

To a solution of D16 (120 mg, 0.26 mmol) in EtOH (20 mL) was added Fe (72 mg). Then a solution of NH₄Cl (68.9 mg) in H₂O (2 mL) was added. The reaction was stirred at 100° C. overnight. The mixture was filtered and the filter cake was washed with EtOH (2×50 mL). The combined filtrate was concentrated to give the title compound as white solid (40 mg, yield 38.9%). LC-MS: 395.3[M+H]⁺. ¹H NMR (400 MHz, CDCl₃): δ 7.85 (s, 1H), 6.07 (s, 1H), 5.68 (br, 1H), 4.50 (d, J=7.6 Hz, 1H), 4.34˜4.29 (m, 1H), 4.12˜4.06 (m, 3H), 3.90 (d, J=2.0 Hz, 1H), 3.52 (t, J=11.6 Hz, 2H), 3.40 (s, 3H), 3.36˜3.27 (m, 2H), 2.32˜2.20 (m, 5H), 1.81˜1.75 (m, 2H).

### Examples E3 and E4

### Enantiomer 1-2: 14-chloro-10-methoxy-4-methyl-5-(oxan-4-yl)-8-oxa-2,5,6,12,16,17-hexaazatricyclo [11.3.1.0^{3,7}]heptadeca-1(16),3,6,13(17),14-pentaene (E3-E4)

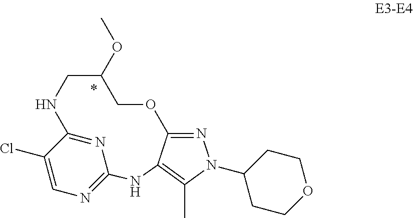 [SMILES: COC1CNc2nc(ncc2Cl)Nc2c(nn(C3CCOCC3)c2C)OC1]

The title compounds E3 (11 mg) and E4 (6 mg) were obtained as white solids from chiral separation of E2 (27 mg)(chiral method A).

E3: LC-MS: 395.4[M+H]⁺. ¹H NMR (400 MHz, CDCl₃): δ 7.75 (s, 1H), 6.57 (s, 1H), 5.73 (br, 1H), 4.44˜4.21 (m, 2H), 4.05˜3.98 (m, 3H), 3.87˜3.81 (m, 1H), 3.47˜3.41 (m, 2H), 3.35 (s, 3H), 3.29˜3.21 (m, 2H), 2.25˜2.14 (m, 5H), 1.75˜1.68 (m, 2H). Chiral RT=4.48 min; ee %=100%.

E4: LC-MS: 395.4[M+H]⁺. ¹H NMR (400 MHz, CDCl₃): δ 7.83 (s, 1H), 6.67 (s, 1H), 5.79 (br, 1H), 4.51˜4.28 (m, 2H), 4.12˜3.06 (m, 3H), 3.94˜3.88 (m, 1H), 3.54˜3.48 (m, 2H), 3.42 (s, 3H), 3.36˜3.28 (m, 2H), 2.31˜2.19 (m, 5H), 1.82˜1.75 (m, 2H). Chiral RT=5.41 min; ee %=100%.

### Example E5

### 14-Chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene (E5)

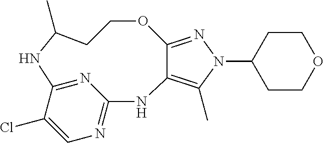 [SMILES: Cc1c2c(nn1C1CCOCC1)OCCC(C)Nc1nc(ncc1Cl)N2]

To a solution of D19 (250 mg, 0.56 mmol) in EtOH (20 mL) and H₂O (4 mL) were added Fe (157 mg, 2.8 mmol) and NH₄Cl (150 mg, 3.8 mmol). The reaction was stirred at 100° C. overnight. The mixture was poured into water and extracted with EtOAc (3×50 mL). The organic layer was concentrated. The crude was washed with CH₃CN to give the title compound as a white solid (97 mg, yield 46.0%). LC-MS: 379.5[M+H]⁺. ¹H NMR (400 MHz, CDCl₃): δ 7.79 (s, 1H), 6.62 (s, 1H), 5.25˜5.23 (m, 1H), 4.50˜4.47 (m, 1H), 4.37˜4.30 (m, 1H), 4.12˜4.04 (m, 4H), 3.54˜3.48 (m, 2H), 2.32˜2.18 (m, 5H), 1.99˜1.75 (m, 4H), 1.33 (d, J=6.4 Hz, 3H).

### Examples E6 and E7

### (11R)-14-Chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene (E6)

### (11S)-14-Chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene (E7)

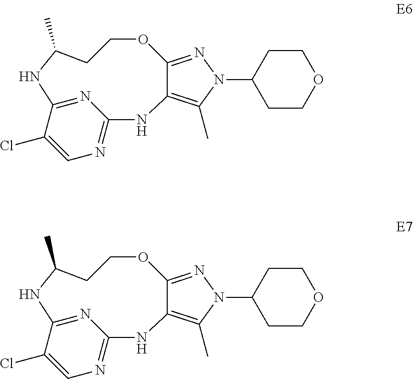 [SMILES: Cc1c2c(nn1C1CCOCC1)OCC[C@@H](C)Nc1nc(ncc1Cl)N2 ; Cc1c2c(nn1C1CCOCC1)OCC[C@H](C)Nc1nc(ncc1Cl)N2]

The title compounds E6 (141 mg) and E7 (148 mg) were obtained as white solid from chiral separation of E5 (410 mg, chiral method A).

E6: LC-MS: 379.5[M+H]⁺. ¹H NMR (400 MHz, CDCl₃): δ 7.82 (s, 1H), 6.10 (s, 1H), 5.14˜5.12 (m, 1H), 4.50˜4.47 (m, 1H), 4.37˜4.31 (m, 1H), 4.12˜4.01 (m, 4H), 3.54˜3.48 (m, 2H), 2.33˜2.21 (m, 5H), 1.89˜1.66 (m, 4H), 1.31˜1.25 (m, 3H). Chiral RT=4.84 min; ee=100%.

E7: LC-MS: 379.5[M+H]⁺. ¹H NMR (400 MHz, CDCl₃): δ 7.82 (s, 1H), 6.06 (s, 1H), 5.13˜5.11 (m, 1H), 4.50˜4.37 (m, 2H), 4.12˜4.00 (m, 4H), 3.54˜3.48 (m, 2H), 2.33˜2.20 (m, 5H), 1.89˜1.66 (m, 4H), 1.33˜1.25 (m, 3H). Chiral RT=6.02 min; ee=98.5%.

### Examples E8 and E9

### 14-Chloro-4-methyl-5-(oxan-3-yl)-8-oxa-2,5,6,12,16,17-hexaazatricyclo[11.3.1.0^{3,7}]heptadeca-1(16),3,6,13(17),14-pentaene (E8)

### 14-Chloro-5-(oxan-3-yl)-8-oxa-2,5,6,12,16,17-hexaazatricyclo[11.3.1.0^{3,7}]heptadeca-1(16),3,6,13(17),14-pentaene (E9)

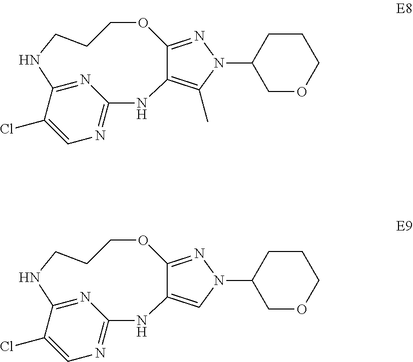 [SMILES: Cc1c2c(nn1C1CCCOC1)OCCCNc1nc(ncc1Cl)N2 ; Clc1cnc2nc1NCCCOc1nn(C3CCCOC3)cc1N2]

To a solution of D28 and D29 (500 mg, 1.1 mmol) in EtOH (10 mL) was added Fe (325 mg, 5.5 mmol) and a solution of NH₄Cl (58 mg, 1.1 mmol) in H₂O (1 mL). The reaction was stirred overnight at 100° C. The mixture was filtered and the filtrate was diluted with H₂O (50 mL). The mixture was extracted with EtOAc (60 mL). The organic layer was washed with brine, dried over anhydrous Na₂SO₄ and concentrated. The crude was washed with CH₃CN and hexane to give the title compounds E8 (50 mg, yield 13.0%) and E9 (41 mg, yield 10.6%) as white solid.

E8: LC-MS: 365.5[M+H]⁺. ¹H NMR (400 MHz, DMSO-d₆): δ 8.29 (s, 1H), 7.75 (s, 1H), 7.29 (br, 1H), 4.18 (br, 2H), 4.04 (m, 1H), 3.83 (m, 2H), 3.48˜3.30 (m, 2H), 2.14 (s, 3H), 2.02˜1.93 (m, 3H), 1.70 (m, 5H).

E9: LC-MS: 351.2[M+H]⁺. ¹H NMR (400 MHz, CDCl₃): δ 7.82 (s, 1H), 7.21 (s, 1H), 6.30 (br, 1H), 5.55 (br, 1H), 4.38 (m, 2H), 4.10˜4.04 (m, 2H), 3.88˜3.85 (m, 1H), 3.68˜3.63 (m, 1H), 3.54˜3.49 (m, 3H), 2.18˜2.03 (m, 2H), 1.95˜1.93 (m, 2H), 1.82˜1.71 (m, 2H)

### Examples E10 and E11

### Enantiomers 1-2: 14-Chloro-4-methyl-5-(oxan-3-yl)-8-oxa-2,5,6,12,16,17-hexaaza tricyclo-[11.3.1.0^{3,7}]heptadeca-1(16),3,6,13(17),14-pentaene (E10 and 11)

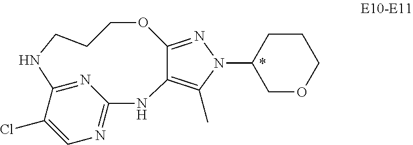 [SMILES: Cc1c2c(nn1C1CCCOC1)OCCCNc1nc(ncc1Cl)N2]

The title compounds E10 (7.8 mg) and E11 (9.6 mg) were obtained as off-white solids from chiral separation of E8 (chiral method A).

E10: LC-MS: 365.2[M+H]⁺. ¹H NMR (400 MHz, CDCl₃): δ 7.80 (s, 1H), 6.36 (s, 1H), 5.58˜5.55 (m, 1H), 4.39˜4.37 (m, 2H), 4.07˜4.00 (m, 1H), 3.96˜3.93 (m, 2H), 3.71˜3.66 (m, 1H), 3.54˜3.46 (m, 2H), 3.45˜3.40 (m 1H), 2.25˜2.17 (m, 3H), 2.06˜2.03 (m, 1H), 1.94˜1.90 (m, 2H), 1.89˜1.80 (m, 3H). Chiral RT=2.885 min; ee=100%.

E11: LC-MS: 365.2[M+H]⁺. ¹H NMR (400 MHz, CDCl₃): δ 7.81 (s, 1H), 6.43 (s, 1H), 5.58˜5.55 (m, 1H), 4.39˜4.37 (m, 2H), 4.07˜4.01 (m, 1H), 3.96˜3.93 (m, 2H), 3.71˜3.66 (m, 1H), 3.53˜3.49 (m, 2H), 3.46˜3.40 (m 1H), 2.25˜2.17 (m, 3H), 2.06˜2.03 (m, 1H), 1.91 (m, 2H), 1.83˜1.80 (m, 3H). Chiral RT=5.694 min; ee=100%.

### Example E12

### 14-Chloro-4-methyl-5-(oxetan-3-yl)-8-oxa-2,5,6,12,16,17-hexaazatricyclo[11.3.1.0^{3,7}]heptadeca-1(16),3,6,13(17),14-pentaene (E12)

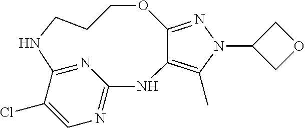 [SMILES: Cc1c2c(nn1C1COC1)OCCCNc1nc(ncc1Cl)N2]

To a solution of D34 (49 mg, 0.122 mmol) in EtOH/H₂O (20 mL/2 mL) were added Fe (35 mg, 0.627 mmol) and NH₄Cl (52 mg, 0.972 mmol). The reaction was stirred overnight under argon at 100° C. The hot mixture was filtered through a pad of Celite and the filtrate was concentrated. The residue was dissolved in EtOAc (20 mL), washed with brine (10 mL), dried over anhydrous Na₂SO₄, filtered and concentrated. The crude was purified by prep-TLC (CH₂Cl₂:MeOH=15:1) to give a yellow solid. The solid was suspended in PE (5 mL) and Et₂O (a few drops). The resultant was sonicated, filtered and the filter cake was dried under infrared lamp, collected to give the title compound as pale yellow solid (16 mg, yield 39%). LC-MS: 337.1[M+H]⁺. ¹H NMR (400 MHz, CDCl₃): δ 7.82 (s, 1H), 6.31 (br, 1H), 5.59 (t, J=5.8 Hz, 1H), 5.32˜5.25 (m, 1H), 5.19 (t, J=6.4 Hz, 2H), 4.91 (t, J=6.8 Hz, 2H), 4.47 (t, J=4.8 Hz, 2H), 3.53 (dd, J=10.8, 6.0 Hz, 2H), 2.16 (s, 3H), 1.94 (t, J=4.4 Hz, 2H).

### Example E13

### 4,14-Dichloro-5-(oxan-4-yl)-8-oxa-2,5,6,12,16,17-hexaazatricyclo[11.3.1.0^{3,7}]heptadeca-1(16),3,6,13(17),14-pentaene (E13)

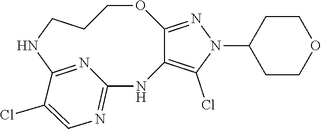 [SMILES: Clc1cnc2nc1NCCCOc1nn(C3CCOCC3)c(Cl)c1N2]

To a solution of D41 (60 mg, 0.14 mmol) in i-PrOH (5 mL) was added conc.HCl (0.012 mL, 0.14 mmol). The reaction was stirred at 80° C. for 6 hrs. The mixture was concentrated and the residue was diluted with EtOAc (20 mL). The mixture was washed with water (20 mL). The combined organic layer was dried over anhydrous Na₂SO₄ and concentrated in vacuo. The crude was washed with CH₃CN and hexane to give the title compound as an off-white solid (23 mg, yield 42%). LC-MS: 385.1 [M+H]⁺. ¹H NMR (400 MHz, CDCl₃): δ 7.85 (s, 1H), 6.25 (s, 1H), 5.57 (s, 1H), 4.39˜4.30 (m, 3H), 4.12˜4.08 (m, 2H), 3.55-3.49 (m, 4H), 2.29-2.19 (m, 2H), 1.96-1.94 (m, 2H), 1.85-1.82 (m, 2H).

### Examples E14 and E15

### 14-Chloro-5-(oxan-4-yl)-8-oxa-2,5,6,12,16,17-hexaazatricyclo[11.3.1.0^{3,7}]heptadeca-1(16),3,6,13(17),14-pentaene-4-carbonitrile (E14)

### 4-Chloro-5-(oxan-4-yl)-8-oxa-2,5,6,12,16,17-hexaazatricyclo[11.3.1.0^{3,7}]heptadeca-1(16),3,6,13(17),14-pentaene-14-carbonitrile (E15)

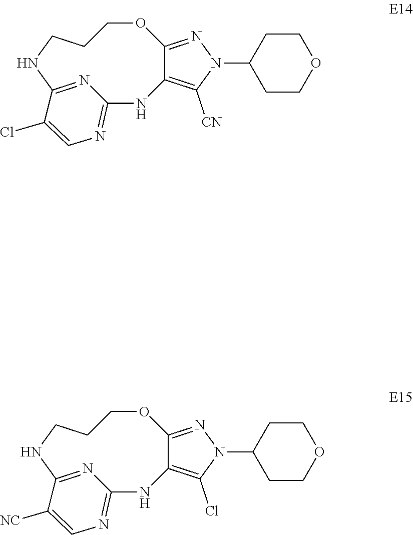 [SMILES: N#Cc1c2c(nn1C1CCOCC1)OCCCNc1nc(ncc1Cl)N2 ; N#Cc1cnc2nc1NCCCOc1nn(C3CCOCC3)c(Cl)c1N2]

A mixture of E13 (110 mg, 0.29 mmol), Pd₂(dba)₃ (28 mg, 0.03 mmol), Ru-phos (14 mg, 0.03 mmol) and Zn(CN)₂ (103 mg, 0.87 mmol) in dioxane (5 mL) was stirred at 100° C. for 2 hrs under microwave. The reaction was poured into ice water (30 mL) and extracted with EtOAc (2×20 mL). The combined organic layer were dried over anhydrous Na₂SO₄ and concentrated. The residue was purified by prep-HPLC to give the title compounds E14 as an off-white solid (2 mg, yield 2%) and E15 as white solid (4 mg, yield 3%).

E14: LC-MS: 376.1[M+H]⁺. ¹H NMR (400 MHz, CDCl₃): δ 7.89 (s, 1H), 6.71 (br, 1H), 5.64 (br, 1H), 4.38˜4.34 (m, 3H), 4.14˜4.10 (m, 2H), 3.56˜3.50 (m, 4H), 2.28˜2.23 (m, 2H), 1.96˜1.92 (m, 4H).

E15: LC-MS: 376.1[M+H]⁺. ¹H NMR (400 MHz, CDCl₃): δ 8.18 (s, 1H), 6.69 (br, 1H), 5.77 (br, 1H), 4.38˜4.34 (m, 3H), 4.13˜4.10 (m, 2H), 3.59˜3.50 (m, 4H), 2.26˜2.23 (m, 2H), 1.98˜1.97 (m, 2H), 1.86˜1.82 (m, 2H).

### Example E16

### 14-Chloro-10-fluoro-4-methyl-5-(oxan-4-yl)-8-oxa-2,5,6,12,16,17-hexaazatricyclo [11.3.1.0^{3,7}]heptadeca-1(16),3,6,13(17),14-pentaene (E16)

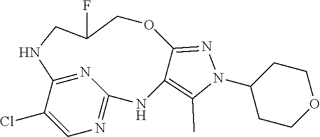 [SMILES: Cc1c2c(nn1C1CCOCC1)OCC(F)CNc1nc(ncc1Cl)N2]

To a solution of D44 (50 mg, 0.119 mmol) in i-PrOH (3 mL) was added p-TsOH (22.68 mg, 0.119 mmol) at 25° C. The reaction was stirred at 125° C. for 1.5 hrs under microwave. The mixture was filtered and the solution was concentrated. The crude was purified by MDAP (base condition) to give the title compound as white solid (40 mg, 88% yield). LC-MS: 383.1[M+H]⁺. ¹H NMR (400 MHz, DMSO-d₆): δ 8.46 (s, 1H), 7.84 (s, 1H), 7.81 (t, J=6.0 Hz, 1H), 4.54˜4.14 (m, 4H), 4.03˜3.87 (m, 2H), 3.82˜3.63 (m, 1H), 3.51˜3.39 (m, 2H), 3.30˜3.23 (m, 1H), 2.17 (s, 3H), 2.06˜1.85 (m, 2H), 1.79˜1.64 (m, 2H).

### Examples E17 and E18

### Enantiomers 1-2: 14-Chloro-10-fluoro-4-methyl-5-(oxan-4-yl)-8-oxa-2,5,6,12,16,17-hexaazatricyclo [11.3.1.0^{3,7}]heptadeca-1(16),3,6,13(17),14-pentaene (E17-E18)

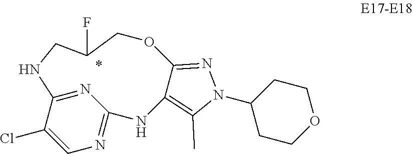 [SMILES: Cc1c2c(nn1C1CCOCC1)OCC(F)CNc1nc(ncc1Cl)N2]

The title compounds E17 (11 mg, yield 27.5%) and E18 (11 mg, yield 27.5%) were prepared from chiral-HPLC separation of E16 (chiral method B).

E17: LC-MS: 383.2[M+H]⁺. ¹H NMR (600 MHz, DMSO-d₆): δ 8.45 (s, 1H), 7.84 (s, 1H), 7.80 (t, J=6.0 Hz, 1H), 4.51˜4.30 (m, 3H), 4.26˜4.16 (m, 1H), 4.02˜3.86 (m, 2H), 3.81˜3.68 (m, 1H), 3.45 (t, J=12.0 Hz, 2H), 3.37 (dd, J=6.0, 15.2 Hz, 1H), 2.17 (s, 3H), 2.05˜1.88 (m, 2H), 1.80˜1.62 (m, 2H). Chiral RT=2.499 min; ee=100%.

E18: LC-MS: 383.2[M+H]⁺. ¹H NMR (600 MHz, DMSO-d₆): δ 8.45 (s, 1H), 7.84 (s, 1H), 7.80 (t, J=6.0 Hz, 1H), 4.49˜4.31 (m, 3H), 4.28˜4.16 (m, 1H), 4.00˜3.90 (m, 2H), 3.80˜3.65 (m, 1H), 3.45 (t, J=12.0 Hz, 2H), 3.40˜3.35 (m, 1H), 2.17 (s, 3H), 2.06˜1.88 (m, 2H), 1.80˜1.64 (m, 2H). Chiral RT=2.865 min; ee=99.3%.

### Examples E19 and E20

### Enantiomers 1-2: 14-chloro-11-ethyl-4-methyl-5-(oxan-4-yl)-8-oxa-2,5,6,12,16,17-hexaazatricyclo[11.3.1.0^{3,7}]heptadeca-1(16),3,6,13(17),14-pentaene (E19-E20)

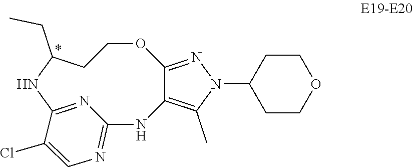 [SMILES: CCC1CCOc2nn(C3CCOCC3)c(C)c2Nc2ncc(Cl)c(n2)N1]

To a solution of D47 (230 mg, 0.501 mmol) in EtOH (28 mL) were added Fe (224 mg, 4.0 mmol) and a solution of NH₄Cl (267 mg, 5.0 mmol) in H₂O (3 mL). The reaction was stirred at 110° C. for 16 hrs. The mixture was filtered and the filter cake was washed with CH₃CN.

The filtrate was concentrated and the crude was purified by flash chromatography on silica gel (CH₂Cl₂:MeOH=60:1 to 30:1) to afford the racemic mixture. The solid was further separated by chiral-HPLC (chiral method B) to give the title compounds E19 (34 mg) as off-white solid and E20 (33 mg) as light yellow solid.

E19: LC-MS: 393.2[M+H]⁺. ¹H NMR (400 MHz, DMSO-d₆): δ 8.29 (s, 1H), 7.78 (s, 1H), 6.74 (d, J=7.6 Hz, 1H), 4.30˜4.11 (m, 3H), 3.96˜3.91 (m, 2H), 3.60˜3.56 (m, 1H), 3.43 (t, J=11.8 Hz, 2H), 2.15 (s, 3H), 2.02˜1.88 (m, 2H), 1.76˜1.65 (m, 4H), 1.58˜1.51 (m, 2H), 0.85 (t, J=7.2 Hz, 3H). Chiral RT=2.179 min; ee=100%.

E20: LC-MS: 393.2[M+H]⁺. ¹H NMR (400 MHz, DMSO-d₆): δ 8.29 (s, 1H), 7.78 (s, 1H), 6.74 (d, J=7.6 Hz, 1H), 4.30˜4.11 (m, 3H), 3.96˜3.91 (m, 2H), 3.60˜3.56 (m, 1H), 3.43 (t, J=12.0 Hz, 2H), 2.15 (s, 3H), 2.02˜1.88 (m, 2H), 1.76˜1.65 (m, 4H), 1.58˜1.51 (m, 2H), 0.85 (t, J=7.2 Hz, 3H). Chiral RT=2.925 min; ee=100%.

### Examples E21-E28

### Enantiomers 1-8: 14-chloro-5-(3-fluorooxan-4-yl)-4,11-dimethyl-8-oxa-2,5,6,12,16,17-hexaazatricyclo [11.3.1.0^{3,7}]heptadeca-1(17),3,6,13,15-pentaene (E21-E28)

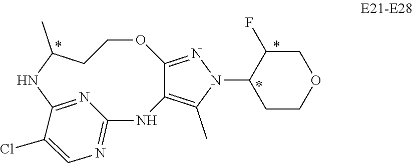 [SMILES: Cc1c2c(nn1C1CCOCC1F)OCCC(C)Nc1nc(ncc1Cl)N2]

To a solution of D53 (330 mg, 0.71 mmol) in EtOH (10 mL) were added Fe (300 mg, 5.35 mmol) and a solution of NH₄Cl (1.2 g, 19 mmol) in H₂O (2 mL). The reaction was stirred at 100° C. for 10 hrs. The crude was extracted with DCM (100 mL) and H₂O (50 mL). The organic layer was concentrated to give white solid (260 mg, 92% yield) which was further separated by chiral-HPLC (chiral method A and C) to give the title compounds E21-E28 as white solids.

E21: 12 mg. LC-MS: 397.2[M+H]⁺. ¹H NMR (400 MHz, CD₃OD): δ 7.73 (s, 1H), 4.79˜4.71 (m, 1H), 4.63˜4.27 (m, 5H), 4.17 (q, J=7.6 Hz, 1H), 4.09˜4.01 (m, 1H), 3.95˜3.91 (m, 1H), 2.40˜2.26 (m, 2H), 2.22 (s, 3H), 1.87˜1.67 (m, 2H), 1.29 (d, J=7.2 Hz, 3H). ¹⁹F NMR (376 MHz, CD₃OD): δ −195.56 (s, 1F). Chiral RT=2.92 min (chiral method A); ee=100%.

E22: 12 mg. LC-MS: 397.2[M+H]⁺. ¹H NMR (400 MHz, CD₃OD): δ 7.73 (s, 1H), 4.82˜4.61 (m, 1H), 4.45˜4.25 (m, 3H), 4.17 (dd, J=5.2, 10.8 Hz, 1H), 4.06˜3.90 (m, 2H), 3.55 (t, J=11.2 Hz, 1H), 3.39 (dt, J=3.6, 10.8 Hz, 1H), 2.37 (dq, J=4.8, 12.6 Hz, 1H), 2.23 (s, 3H), 1.99 (d, J=13.2 Hz, 1H), 1.87˜1.65 (m, 2H), 1.29 (d, J=7.2 Hz, 3H). ¹⁹F NMR (376 MHz, CD₃OD): δ −195.56 (s, 1F). Chiral RT=3.252 min (chiral method A); ee=99.5%;

E23: 13 mg. LC-MS: 397.2[M+H]⁺. ¹H NMR (400 MHz, CD₃OD): δ 7.73 (s, 1H), 4.79˜4.68 (m, 1H), 4.61˜4.28 (m, 4H), 4.27˜4.13 (m, 2H), 4.05 (q, J=7.6 Hz, 1H), 3.99˜3.89 (m, 1H), 2.53˜2.30 (m, 2H), 2.21 (s, 3H), 1.87˜1.66 (m, 2H), 1.29 (d, J=7.2 Hz, 3H). ¹⁹F NMR (376 MHz, CD₃OD): δ −232.35 (s, 1F). Chiral RT=3.720 min (chiral method A); ee=100%;

E24: 12 mg. LC-MS: 397.1[M+H]⁺. ¹H NMR (400 MHz, CD₃OD): δ 7.73 (s, 1H), 4.81˜4.74 (m, 1H), 4.47˜4.28 (m, 3H), 4.19 (dd, J=5.2, 10.8 Hz, 1H), 4.05˜3.87 (m, 2H), 3.54 (t, J=11.2 Hz, 1H), 3.41 (dt, J=3.6, 10.8 Hz, 1H), 2.33˜2.17 (m, 4H), 1.99˜1.65 (m, 3H), 1.31 (d, J=6.8 Hz, 3H). ¹⁹F NMR (377 MHz, CD₃OD): δ −195.18 (s, 1F). Chiral RT=3.887 min (method A), 2.195 min (method C); ee=99.1%;

E25: 11 mg. LC-MS: 397.2[M+H]⁺. ¹H NMR (400 MHz, CD₃OD): δ 7.73 (s, 1H), 4.79˜4.70 (m, 1H), 4.64˜4.27 (m, 5H), 4.21˜4.12 (m, 1H), 4.09˜4.00 (m, 1H), 3.98˜3.85 (m, 1H), 2.40˜2.27 (m, 2H), 2.22 (s, 3H), 1.86˜1.70 (m, 2H), 1.29 (d, J=6.8 Hz, 3H). ¹⁹F NMR (376 MHz, CD₃OD): δ −232.14 (s, 1F). Chiral RT=3.887 min (method A), 2.606 min (method C); ee=99.1%;

E26: 16 mg. LC-MS: 397.2[M+H]⁺. ¹H NMR (400 MHz, CD₃OD): δ 7.74 (s, 1H), 4.82˜4.72 (m, 1H), 4.48˜4.25 (m, 3H), 4.19 (dd, J=5.2, 10.8 Hz, 1H), 3.98 (dd, J=3.2, 7.6 Hz, 2H), 3.54 (t, J=11.2 Hz, 1H), 3.41 (dt, J=3.6, 10.8 Hz, 1H), 2.36˜2.17 (m, 4H), 1.99˜1.70 (m, 3H), 1.31 (d, J=6.8 Hz, 3H). ¹⁹F NMR (376 MHz, CD₃OD): δ −195.18 (s, 1F). Chiral RT=4.794 min (chiral method A); ee %=100%.

E27: 13 mg. LC-MS: 397.2[M+H]⁺. ¹H NMR (400 MHz, CD₃OD): δ 7.74 (s, 1H), 4.79˜4.71 (m, 1H), 4.62˜4.28 (m, 4H), 4.27˜4.12 (m, 2H), 4.06 (q, J=7.6 Hz, 1H), 4.00˜3.84 (m, 1H), 2.52˜2.30 (m, 2H), 2.22 (s, 3H), 1.87˜1.67 (m, 2H), 1.30 (d, J=6.8 Hz, 3H). ¹⁹F NMR (376 MHz, CD₃OD): δ −232.35 (s, 1F). Chiral RT=5.616 min (chiral method A); ee %=97.5%.

E28: 11 mg. LC-MS: 397.2[M+H]⁺. ¹H NMR (400 MHz, CD₃OD): δ 7.73 (s, 1H), 4.81˜4.61 (m, 1H), 4.45˜4.26 (m, 3H), 4.17 (dd, J=5.2, 10.8 Hz, 1H), 4.05˜3.87 (m, 2H), 3.55 (t, J=11.2 Hz, 1H), 3.39 (dt, J=3.6, 10.8 Hz, 1H), 2.37 (dq, J=4.8, 12.6 Hz, 1H), 2.23 (s, 3H), 2.05˜1.94 (m, 1H), 1.86˜1.69 (m, 2H), 1.29 (d, J=6.8 Hz, 3H). ¹⁹F NMR (376 MHz, CD₃OD): δ −195.56 (s, 1F). Chiral RT=6.330 min (chiral method A); ee %=100%.

### Examples E29 and E30

### Enantiomers 1-2: 14-bromo-4,11-dimethyl-5-(oxan-4-yl)-8-oxa-2,5,6,12,16,17-hexaaza tricyclo[11.3.1.0^{3,7}]heptadeca-1(16),3,6,13(17),14-pentaene (E29-E30)

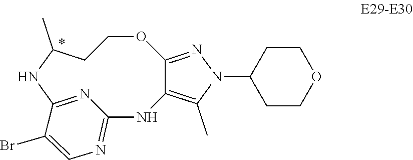 [SMILES: Cc1c2c(nn1C1CCOCC1)OCCC(C)Nc1nc(ncc1Br)N2]

To a solution of D56 (280 mg, 0.6 mmol) in EtOH (50 mL) were added Fe (168 mg, 3 mmol) and a solution of NH₄Cl (160.4 mg, 3 mmol) in H₂O (5 mL). The reaction was stirred overnight at 100° C. The mixture was filtered and the filter cake was washed with MeOH (3×100 mL). The combined filtrate was concentrated and the residue was poured into the sat.NaHCO₃. The mixture was then extracted with EtOAc (3×100 mL). The combined organic layers were washed with brine, dried over anhydrous Na₂SO₄ and concentrated to give the racemic product (230 mg, yield 95%). The solid was further separated by chiral-HPLC (chiral method B) to give the title compounds E29 (70 mg) and E30 (69 mg) as off-white solids.

E29: LC-MS: 423.2 [M+H]⁺. ¹H NMR (400 MHz, CD₃OD): δ 7.82 (s, 1H), 4.41˜4.33 (m, 1H), 4.30˜4.23 (m, 2H), 4.06˜4.02 (m, 2H), 3.94˜3.93 (m, 1H), 3.59˜3.53 (m, 2H), 2.22˜2.10 (m, 5H), 1.83˜1.72 (m, 4H), 1.30˜1.28 (d, J=7.6 Hz, 3H). Chiral RT=2.336 min; ee=100%;

E30: LC-MS: 423.1 [M+H]⁺. ¹H NMR (400 MHz, CD₃OD): δ 7.82 (s, 1H), 4.41˜4.38 (m, 1H), 4.32˜4.23 (m, 2H), 4.06˜4.03 (m, 2H), 3.95˜3.93 (m, 1H), 3.59˜3.53 (m, 2H), 2.25˜2.10 (m, 5H), 1.83˜1.74 (m, 4H), 1.30˜1.28 (m, 3H). Chiral RT=3.056 min; ee=100%;

### Examples E31, E32, E33 and E34

### Enantiomers 1-4: 14-chloro-4,10,11-trimethyl-5-(oxan-4-yl)-8-oxa-2,5,6,12,16,17-hexaazatricyclo [11.3.1.0^{3,7}]heptadeca-1(16),3,6,13(17),14-pentaene (E31-E34)

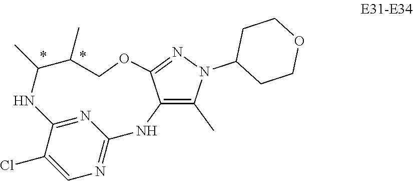 [SMILES: Cc1c2c(nn1C1CCOCC1)OCC(C)C(C)Nc1nc(ncc1Cl)N2]

A mixture of D63 (220 mg, 0.48 mmol), NH₄Cl (220 mg) and Fe (220 mg) in EtOH/H₂O (20 mL/5 mL) was stirred was 100° C. for 16 hrs. Then aq.NaHCO₃(5 mL) was added dropwise. The mixture was filtered and the filtrate was concentrated. The residue was diluted with CH₂Cl₂ (30 mL), dried over anhydrous Na₂SO₄, filtered and concentrated. The crude was washed with CH₃CN to give the racemic product (180 mg, yield 96%) as an off-white solid, which was chiral separated (chiral method A) to give the title compounds E31-E34.

E31: pink solid, 34 mg. LC-MS: 393.2[M+H]⁺. ¹H NMR (400 MHz, CDCl₃): δ 7.81 (s, 1H), 6.07 (br, 1H), 4.84 (br, 1H), 4.32˜4.26 (m, 3H), 4.12˜4.04 (m, 3H), 3.53˜3.47 (m, 2H), 2.33˜2.19 (m, 5H), 1.91 (br, 1H), 1.81˜1.73 (m, 2H), 1.30 (d, J=7.2 Hz, 3H), 0.82 (d, J=7.2 Hz, 3H). Chiral RT=1.596 min; ee=100%.

E32: pink solid, 28 mg. LC-MS: 393.2[M+H]⁺. ¹H NMR (400 MHz, CDCl₃): δ 7.82 (s, 1H), 6.09 (br, 1H), 5.15 (br, 1H), 4.31˜4.25 (m, 2H), 4.23˜4.03 (m, 4H), 3.53˜3.47 (m, 2H), 2.34˜2.20 (m, 5H), 2.08˜2.02 (m, 1H), 1.83˜1.74 (m, 2H), 1.18 (d, J=7.2 Hz, 3H), 0.93 (d, J=7.6 Hz, 3H). Chiral RT=2.159 min; ee=100%.

E33: white solid, 32 mg. LC-MS: 393.2[M+H]⁺. ¹H NMR (400 MHz, CDCl₃): δ 7.81 (s, 1H), 6.07 (br, 1H), 4.84 (br, 1H), 4.32˜4.26 (m, 3H), 4.12˜4.04 (m, 3H), 3.53˜3.47 (m, 2H), 2.33˜2.19 (m, 5H), 1.91 (s, 1H), 1.81˜1.73 (m, 2H), 1.30 (d, 3H, J=7.2 Hz), 0.82 (d, 3H, J=7.2 Hz). Chiral RT=3.877 min; ee=100%.

E34: white solid, 30 mg. LC-MS: 393.2[M+H]⁺. ¹H NMR (400 MHz, CDCl₃): δ 7.83 (s, 1H), 6.00 (br, 1H), 5.15 (br, 1H), 4.29˜4.29 (m, 2H), 4.13˜4.07 (m, 4H), 3.54˜3.47 (m, 2H), 2.28˜2.01 (m, 5H), 2.04 (br, 1H), 1.78˜1.77 (m, 2H), 1.75 (d, J=7.6 Hz, 3H), 0.93 (d, J=8.0 Hz, 3H). Chiral RT=8.911 min; ee=100%.

### Examples E35 and E36

### Enantiomers 1-2: 14-chloro-11-(methoxymethyl)-4-methyl-5-(oxan-4-yl)-8-oxa-2,5,6, 12,16,17-hexaazatricyclo-[11.3.1.0^{3,7}]heptadeca-1(16),3,6,13(17),14-pentaene (E35-E36)

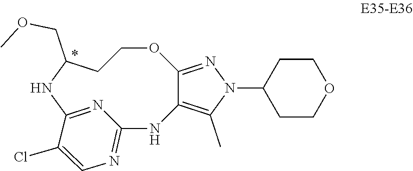 [SMILES: COCC1CCOc2nn(C3CCOCC3)c(C)c2Nc2ncc(Cl)c(n2)N1]

To a solution of D68 (247 mg, 0.520 mmol) in EtOH/H₂O (80 mL/20 mL) were added Fe (146 mg, 2.61 mmol) and NH₄Cl (139 mg, 2.60 mmol). The reaction was stirred at 105° C. for 16 hrs. Aq. Na₂CO₃ was added and the mixture was filtered. The filter cake was washed with CH₂Cl₂/MeOH=20:1 and the filtrate was concentrated. The residue was dissolved in DCM and filtered. The filtrate was concentrated to give the racemic product (180 mg, yield 61%). The title compounds E35 (62 mg) and E36 (77 mg) were prepared from chiral separation (chiral method A) as white solids.

E35: LC-MS: 409.2[M+H]⁺. ¹H NMR (400 MHz, CDCl₃): δ 7.82 (s, 1H), 6.17 (s, 1H), 5.68 (d, J=7.2 Hz, 1H), 4.50˜4.47 (m, 1H), 4.33 (t, J=11.2 Hz, 1H), 4.12˜4.04 (m, 4H), 3.54˜3.45 (m, 4H), 3.41 (s, 3H), 2.31˜2.20 (m, 2H), 2.20 (s, 3H), 2.07˜2.01 (m, 1H), 1.82˜1.75 (m, 3H). Chiral RT=3.129 min; ee=100%;

E36: LC-MS: 409.2[M+H]⁺. ¹H NMR (400 MHz, CDCl₃): δ 7.82 (s, 1H), 6.14 (s, 1H), 5.69 (d, J=7.2 Hz, 1H), 4.49 (dd, J=11.2, 3.6 Hz, 1H), 4.33 (t, J=11.2 Hz, 1H), 4.12˜4.04 (m, 4H), 3.54˜3.45 (m, 4H), 3.41 (s, 3H), 2.31˜2.20 (m, 2H), 2.20 (s, 3H), 2.07˜2.01 (m, 1H), 1.83˜1.74 (m, 3H). Chiral RT=12.099 min; ee=100%;

### Examples E37 and E38

### Enantiomers 1-2: Ethyl 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanoate (E37-E38)

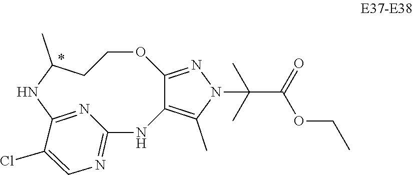 [SMILES: CCOC(=O)C(C)(C)n1nc2c(c1C)Nc1ncc(Cl)c(n1)NC(C)CCO2]

To a solution of D74 (750 mg) and Fe (750 mg) in 95% EtOH (60 mL) was added NH₄Cl (1.6 g). The reaction was heated to 90° C. and stirred overnight. The mixture was filtered and the filtrate was concentrated. give the racemic product (700 mg, crude). The title compounds E37 (8 mg) and E38 (4 mg) were prepared from chiral separation of racemic compound (52 mg) (Chiral method D) as white solids.

E37: LC-MS: 409.3[M+H]⁺. ¹H NMR (400 MHz, CDCl₃): δ 7.76 (s, 1H), 7.17 (br, 1H), 5.39 (d, J=6.8 Hz, 1H), 4.47 (dd, J=2.4, 11.6 Hz, 1H), 4.37˜4.28 (m, 1H), 4.23 (q, J=6.8 Hz, 2H), 4.14˜3.92 (m, 1H), 2.13 (s, 3H), 1.92˜1.91 (m, 2H), 1.76 (s, 3H), 1.75 (s, 3H), 1.35 (d, J=6.8 Hz, 3H), 1.27 (t, J=6.8 Hz, 3H). Chiral RT=2.41 min; ee=100%.

E38: LC-MS: 409.3[M+H]⁺. ¹H NMR (400 MHz, CDCl₃): δ 7.75 (s, 1H), 7.31 (br, 1H), 5.42 (d, J=6.4 Hz, 1H), 4.55˜4.41 (m, 1H), 4.38˜4.28 (m, 1H), 4.23 (q, J=6.8 Hz, 2H), 4.09 (m, 1H), 2.13 (s, 3H), 1.92˜1.91 (m, 2H), 1.76 (s, 3H), 1.75 (s, 3H), 1.35 (d, J=6.8 Hz, 3H), 1.27 (t, J=6.8 Hz, 3H). Chiral RT=10.99 min; ee=100%.

### Examples E39 and E40

### Enantiomers 1-2: 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanoic acid (E39-E40)

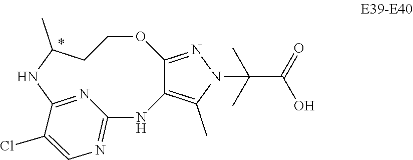 [SMILES: Cc1c2c(nn1C(C)(C)C(=O)O)OCCC(C)Nc1nc(ncc1Cl)N2]

To a solution of ethyl 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanoate (600 mg) in 95% EtOH (50 mL) was added LiOH H₂O (200 mg). The reaction was heated to 50° C. and stirred for 5 hrs. 1 M HCl solution (5 mL) was added and the solid formed was filtered, dried to give the racemic product (600 mg, crude), which (120 mg) was chiral separated (Chiral method E) to give the title compounds E39 (8 mg) and E40 (8 mg) as white solids.

E39: LC-MS: 381.2[M+H]⁺. ¹H NMR (400 MHz, CDCl₃): δ 10.16 (s, 1H), 8.01 (s, 1H), 7.69 (s, 1H), 6.04 (s, 1H), 4.47 (m, 1H), 4.27˜4.10 (m, 2H), 2.17 (s, 3H), 2.00˜1.92 (m, 2H), 1.80 (s, 3H), 1.79 (s, 3H) 1.41 (d, J=7.2 Hz, 3H). Chiral RT=5.59 min; ee=100%.

E40: LC-MS: 381.2[M+H]⁺. ¹H NMR (400 MHz, CDCl₃): δ 8.02 (s, 1H), 7.07 (s, 1H), 6.06 (s, 1H), 4.45 (m, 1H), 4.25˜4.09 (m, 2H), 2.17 (s, 3H), 2.02˜1.92 (m, 2H), 1.80 (s, 3H), 1.79 (s, 3H), 1.41 (d, J=6.8 Hz, 3H). Chiral RT=6.65 min; ee=100%.

### Examples E41 and E42

### Enantiomers 1-2: 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanamide (E41-E42)

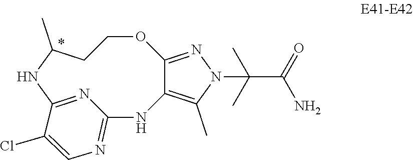 [SMILES: Cc1c2c(nn1C(C)(C)C(N)=O)OCCC(C)Nc1nc(ncc1Cl)N2]

To a solution of 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanoic acid (200 mg, 0.52 mmol) in DMF (20 mL) was added TBTU (202 mg, 0.62 mmol) and Et₃N (127 mg, 1.25 mmol). After stirred at room temperature for 20 min, 7 M NH₃/THF solution (2 mL) was added and the mixture was stirred at room temperature for another 3 hrs. The mixture was then diluted water (100 mL), extracted with EtOAc (2×100 mL), dried over Na₂SO₄, filtered and concentrated to get the racemic product (120 mg, crude). The title compound E41 (3.1 mg and E42 (3.1 mg) were obtained as white solids by chiral separation of the racemic product (40 mg) (chiral method D).

E41: LC-MS: 380.3[M+H]⁺. ¹H NMR (400 MHz, CDCl₃): δ 7.78 (s, 1H), 7.55˜7.37 (m, 1H), 5.46 (br, 2H), 5.36 (br, 1H), 4.63˜4.26 (m, 2H), 4.15˜3.91 (m, 1H), 2.26 (s, 3H), 1.99˜1.86 (m, 2H), 1.80 (s, 3H), 1.79 (s, 3H), 1.38 (d, J=6.8 Hz, 2H). Chiral RT=5.79 min; ee=100%.

E42: LC-MS: 380.3[M+H]⁺. ¹H NMR (400 MHz, CDCl₃): δ 8.18 (s, 1H), 7.75 (br, 1H), 5.65 (d, J=6.0 Hz, 1H), 5.48 (br, 1H), 5.34 (br, 1H), 4.59˜4.21 (m, 2H), 4.16˜3.89 (m, 1H), 2.27 (s, 3H), 1.97˜1.85 (m, 2H), 1.80 (s, 3H), 1.78 (s, 3H), 1.40 (d, J=6.8 Hz, 3H). Chiral RT=8.06 min; ee=100%.

### Examples E43 and E44

### Enantiomers 1-2: 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanenitrile (E43-E44)

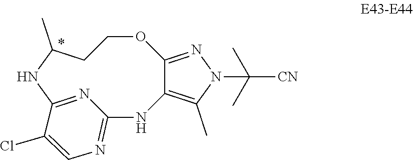 [SMILES: Cc1c2c(nn1C(C)(C)C#N)OCCC(C)Nc1nc(ncc1Cl)N2]

To a solution of 2-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}-2-methylpropanamide (80 mg, 0.22 mmol) in DCM (20 mL) was added Et₃N (106 mg, 1.05 mmol). After stirring at 0° C. for 10 min, TFAA (110 mg, 0.57 mmol) was added drop wise and the mixture was stirred at room temperature for 3 hrs. The mixture was diluted with NaHCO₃ solution (80 mL). The organic layer was dried and concentrated to get the racemic product (70 mg, crude). The title compound E43 (18 mg) and E44 (14 mg) were obtained as white solids by chiral separation of the racemic product (chiral method A).

E43: LC-MS: 362.2[M+H]⁺. ¹H NMR (400 MHz, CDCl₃): 7.79 (s, 1H), 7.03 (br, 1H), 5.35 (br, 1H), 4.52˜4.22 (m, 2H), 4.05 (m, 1H), 2.48 (s, 3H), 1.99 (s, 3H), 1.90 (s, 3H), 1.88˜1.70 (m, 2H), 1.35 (d, J=6.8 Hz, 3H). Chiral RT=2.73 min; ee=100%.

E44: LC-MS: 362.2[M+H]⁺. ¹H NMR (400 MHz, CDCl₃): δ 7.81 (s, 1H), 6.67 (br, 1H), 5.27 (br, 1H), 4.52˜4.23 (m, 2H), 4.04 (m, 1H), 2.47 (s, 3H), 1.99 (s, 3H), 1.90 (s, 3H), 1.88˜1.76 (m, 2H), 1.34 (d, J=6.8 Hz, 3H). Chiral RT=6.41 min; ee=100%.

### Example E45

### 14-Chloro-4-methyl-5-[3-(morpholin-4-yl)cyclobutyl]-8-oxa-2,5,6,12,16,17-hexaaza tricyclo[11.3.1.0^{3,7}]heptadeca-1(16),3,6,13(17),14-pentaene (E45)

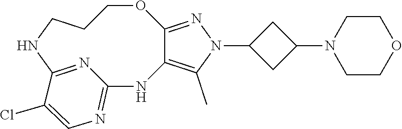 [SMILES: Cc1c2c(nn1C1CC(N3CCOCC3)C1)OCCCNc1nc(ncc1Cl)N2]

To a solution of D76 (195 mg, 0.401 mmol) and Fe (112 mg, 2.01 mmol) in EtOH/H₂O (20 mL/4 mL) was added NH₄Cl (107 mg, 2.00 mmol). The reaction was heated to 100° C. and stirred overnight. NaHCO₃ solution was added, the mixture was filtered and the filtrate was concentrated. The residue was suspended in CH₂Cl₂, dried over anhydrous Na₂SO₄, filtered and concentrated to give the title compound as a white solid (69.3 mg, yield 41%). LC-MS: 420.2 [M+H]⁺. ¹H NMR (400 MHz, DMSO-d₆): δ 8.30 (s, 1H), 7.76 (s, 1H), 7.32 (t, J=5.8 Hz, 1H), 4.43˜4.32 (m, 1H), 4.23 (br, 2H), 3.58 (br, 4H), 3.28 (d, J=4.8 Hz, 2H), 2.45˜2.40 (m, 3H), 2.28˜2.21 (m, 6H), 2.10 (s, 3H), 1.72 (br, 2H).

### Example E46

### (11R)-14-chloro-4,11-dimethyl-5-[(3S)-oxolan-3-yl]-8-oxa-2,5,6,12,16,17-hexaaza tricyclo [11.3.1.0^{3,7}]heptadeca-1(16),3,6,13(17),14-pentaene (E46)

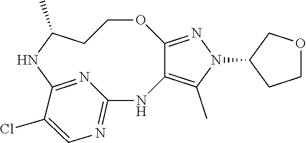 [SMILES: Cc1c2c(nn1[C@H]1CCOC1)OCC[C@@H](C)Nc1nc(ncc1Cl)N2]

A solution of D80 (150 mg, 0.35 mmol), Fe (150 mg, 2.68 mmol) and NH₄Cl (300 mg, 5.61 mmol) in EtOH/H₂O (25 mL, 3:1) was heated to 90° C. for 16 hrs. Then sat.NaHCO₃(3 mL) was added and the mixture was stirred for 10 min. The mixture was filtered and the filter cake was washed with DCM. The filtrate was concentrated and the residue was re-dissolved in DCM (50 mL). After stirring for 30 min, the mixture was filtered and concentrated. The crude was purified by column chromatography on silica gel (PE:EA=3:1˜1:1) to give the title compound as an off-white solid (47 mg, yield 37%). LC-MS: 365.1[M+H]⁺. ¹H NMR (400 MHz, CDCl₃): δ 7.78 (s, 1H), 7.24˜7.20 (m, 1H), 6.18 (m, 1H), 5.36˜5.34 (m, 1H), 4.74˜4.71 (m, 1H), 4.48˜4.44 (m, 1H), 4.35˜4.30 (m, 1H), 4.08˜4.03 (m, 3H), 3.96˜3.91 (m, 1H), 2.35˜2.26 (m, 2H), 2.23 (s, 3H), 1.89˜1.81 (m, 2H), 1.31 (d, J=7.2 Hz, 3H).

### Example E47

### (11R)-14-chloro-4,11-dimethyl-5-[(3R)-oxolan-3-yl]-8-oxa-2,5,6,12,16,17-hexaaza tricyclo [11.3.1.0^{3,7}]heptadeca-1(16),3,6,13(17),14-pentaene (E47)

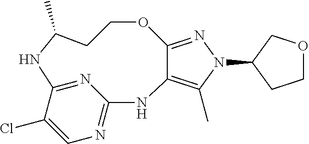 [SMILES: Cc1c2c(nn1[C@@H]1CCOC1)OCC[C@@H](C)Nc1nc(ncc1Cl)N2]

A solution of D81 (130 mg, 0.30 mmol), Fe (130 mg, 2.32 mmol) and NH₄Cl (260 mg, 4.86 mmol) in EtOH/H₂O (25 mL, 3:1) was heated to 90° C. for 16 hrs. Then sat.NaHCO₃(3 mL) was added and the mixture was stirred for 10 min. The mixture was filtered and the filter cake was washed with DCM. The filtrate was concentrated and the residue was re-dissolved in DCM (50 mL). After stirring for 10 min, the mixture was filtered and concentrated. The crude was purified by column chromatography on silica gel (PE:EA=5:1˜1:1) to give the title compound as an off-white solid (41 mg, yield 37%). LC-MS: 365.2[M+H]⁺. ¹H NMR (400 MHz, CDCl₃): δ 8.20˜8.25 (m, 1H), 7.75 (s, 1H), 5.59 (d, J=6.4 Hz 1H), 4.73˜4.71 (m, 1H), 4.48˜4.44 (m, 1H), 4.35˜4.30 (m, 1H), 4.08˜4.03 (m, 3H), 3.96˜3.91 (m, 2H), 2.35˜2.26 (m, 2H), 2.23 (s, 3H), 1.89˜1.81 (m, 2H), 1.32 (d, J=7.2 Hz, 3H).

### Examples E48 and E49

### Isomers 1-2: 14-chloro-4-methyl-5-{4-[(1R,4R)-2-oxa-5-azabicyclo[2.2.1]heptan-5-yl]cyclohexyl}-8-oxa-2,5,6,12,16,17-hexaazatricyclo[11.3.1.0^{3,7}]heptadeca-1(16),3,6,13(17),14-pentaene (E48-E49)

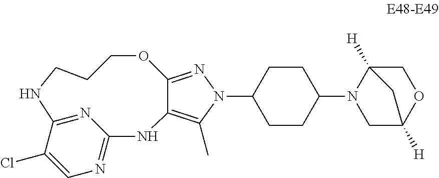 [SMILES: Cc1c2c(nn1C1CCC(N3C[C@H]4C[C@@H]3CO4)CC1)OCCCNc1nc(ncc1Cl)N2]

To a mixture of D85 (100 mg, 0.265 mmol), (1R,4R)-2-oxa-5-azabicyclo[2.2.1]heptane hydrochloride (43 mg, 0.317 mmol), 4 Å molecular sieves (100 mg), AcOH (10 mg) in CH₂Cl₂ (5 mL) under argon at 0° C. was added NaBH₃CN (35 mg, 0.557 mmol). The reaction was stirred for 15 hrs at room temperature. The mixture was filtered and aq. NaHCO₃ (20 mL) was added. The resultant was extracted with CH₂Cl₂ (2×15 mL). The combined organics were washed with brine (20 mL), dried over anhydrous Na₂SO₄, filtered and concentrated. The crude was purified by prep-TLC (CH₂Cl₂:MeOH=8:1) to give the mixture as yellow solids (71 mg, yield 58%). The title compounds E48 (8 mg) and E49 (34 mg) were obtained as white solids from chiral separation of the mixture (chiral method A).

E48: LCMS: 460.3[M+H]⁺. ¹H NMR (400 MHz, CDCl₃): δ 7.83 (s, 1H), 6.13 (s, 1H), 5.55 (t, J=6.0 Hz, 1H), 4.54˜4.27 (m, 3H), 4.08 (d, J=7.6 Hz, 1H), 3.98˜3.84 (m, 1H), 3.71˜3.61 (m, 2H), 3.54 (q, J=6.0 Hz, 2H), 3.09 (d, J=9.6 Hz, 1H), 2.73 (br, 1H), 2.46˜2.25 (m, 3H), 2.22 (s, 3H), 1.94 (m, 5H), 1.77 (d, J=9.6 Hz, 1H), 1.70˜1.48 (m, 4H). Chiral RT=4.532 min.

E49: LCMS: 460.4[M+H]⁺. ¹H NMR (400 MHz, CDCl₃): δ 7.83 (s, 1H), 6.10 (s, 1H), 5.55 (t, J=6.0 Hz, 1H), 4.53˜4.22 (m, 3H), 4.07 (d, J=8.0 Hz, 1H), 3.87 (t, J=11.6 Hz, 1H), 3.75 (br, 1H), 3.65 (d, J=7.6 Hz, 1H), 3.54 (q, J=6.0 Hz, 2H), 3.13 (d, J=9.6 Hz, 1H), 2.50 (d, J=9.6 Hz, 2H), 2.21 (s, 3H), 2.14˜1.86 (m, 9H), 1.84˜1.75 (m, 1H), 1.43˜1.17 (m, 2H). Chiral RT=6.286 min.

### Examples E50 and E51

### Isomers 1-2: 14-chloro-4-methyl-5-{4-[(1S,4S)-2-oxa-5-azabicyclo[2.2.1]heptan-5-yl]cyclohexyl}-8-oxa-2,5,6,12,16,17-hexaazatricyclo[11.3.1.0^{3,7}]heptadeca-1(16),3,6,13(17),14-pentaene (E50-E51)

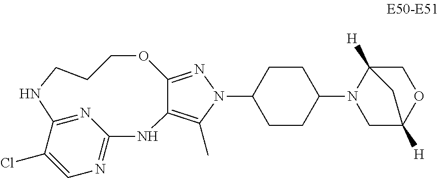 [SMILES: Cc1c2c(nn1C1CCC(N3C[C@@H]4C[C@H]3CO4)CC1)OCCCNc1nc(ncc1Cl)N2]

To a solution of D85 (100 mg, 0.265 mmol), (1S,4S)-2-oxa-5-azabicyclo[2.2.1]heptane hydrochloride (43 mg, 0.319 mmol), 4 Å molecular sieves (100 mg), AcOH (10 mg) in CH₂Cl₂ (5 mL) under argon at 0° C. was added NaBH₃CN (35 mg, 0.557 mmol). The reaction was stirred for 15 hrs at room temperature. The mixture was filtered and aq. NaHCO₃ (20 mL) was added. The organic layer was concentrated and the crude was purified by prep-TLC (CH₂Cl₂:MeOH=8:1) to give the mixture as a yellow solid (88 mg, yield 72%). The title compounds E50 (10 mg) and E51 (21 mg) were obtained as white solids from chiral separation of the mixture (chiral method F).

E50: LC-MS: 460.3 [M+H]⁺. ¹H NMR (400 MHz, CDCl₃): δ 7.81 (s, 1H), 6.07 (br, 1H), 5.53 (br, 1H), 4.40 (br, 3H), 4.05 (d, J=8.0 Hz, 1H), 3.85 (br, 1H), 3.74 (br, 1H), 3.68˜3.46 (m, 3H), 3.11 (d, J=9.6 Hz, 1H), 2.48 (d, J=9.2 Hz, 2H), 2.19 (s, 3H), 2.13˜1.82 (m, 9H), 1.83˜1.72 (m, 1H), 1.40˜1.18 (m, 2H). Chiral RT=4.549 min.

E51: LC-MS: 460.3 [M+H]⁺. ¹H NMR (400 MHz, CDCl₃): δ 7.81 (br, 1H), 6.07 (br, 1H), 5.53 (br, 1H), 4.40 (br, 3H), 4.06 (d, J=7.6 Hz, 1H), 3.91 (br, 1H), 3.70˜3.58 (m, 2H), 3.52 (d, J=4.8 Hz, 2H), 3.07 (d, J=8.4 Hz, 1H), 2.72 (br, 1H), 2.44˜2.23 (m, 3H), 2.23˜2.14 (m, 4H), 1.93 (br, 5H), 1.68˜1.43 (m, 4H). Chiral RT=5.341 min.

### Example E52

### (11R)-14-chloro-4,11-dimethyl-5-(oxetan-3-yl)-8-oxa-2,5,6,12,16,17-hexaazatricyclo [11.3.1.0^{3,7}]heptadeca-1(16),3,6,13(17),14-pentaene (E52)

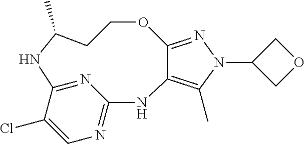 [SMILES: Cc1c2c(nn1C1COC1)OCC[C@@H](C)Nc1nc(ncc1Cl)N2]

To a solution of D109 (6.5 g, 15.58 mmol) in HOAc/MeOH (6 mL/60 mL) was added Zn (3.1 g, 46.73 mmol). The reaction was stirred at 80° C. for 16 hrs. The mixture was filtered and the filtrate was concentrated. The residue was dissolved in CH₂Cl₂ (150 mL) and washed with sat.NaHCO₃(2×60 mL). The organic layers were dried, filtered and concentrated. The crude was purified by column chromatography on silica gel (CH₂Cl₂:MeOH=50:1) to give a yellow solid (2.7 g). The solid was washed with CH₃CN and EtOH, recrystallized (CH₃CN/MeOH then CH₂Cl₂/hexane) to give the title compound as a white solid (580 mg, yield 11%). LC-MS: 351.2[M+H]⁺. ¹H NMR (400 MHz, CDCl₃): δ 7.80 (s, 1H), 6.61 (br, 1H), 5.32˜5.16 (m, 4H), 4.93˜4.88 (m, 2H), 4.54˜4.43 (m, 2H), 4.05˜4.01 (m, 1H), 2.16 (s, 3H), 1.95˜1.82 (m, 2H), 1.34 (d, J=7.2 Hz, 3H).

### Examples E53 and E54

### Enantiomers 1-2: 14-chloro-11-(fluoromethyl)-4-methyl-5-(oxan-4-yl)-8-oxa-2,5,6,12, 16,17-hexaazatricyclo[11.3.1.0^{3,7}]heptadeca-1(16),3,6,13(17),14-pentaene (E53-E54)

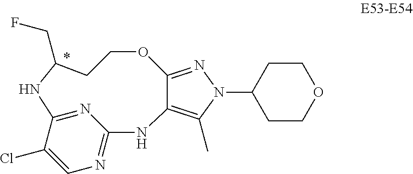 [SMILES: Cc1c2c(nn1C1CCOCC1)OCCC(CF)Nc1nc(ncc1Cl)N2]

To a solution of D91 (480 mg, 1.04 mmol) in EtOH/H₂O (58 mL/6 mL) were added Fe (463 mg, 8.29 mmol) and NH₄Cl (554 mg, 10.4 mmol) at room temperature. The reaction was stirred under argon at 110° C. for 18 hrs. The reaction mixture was cooled to room temperature and then quenched with aq. Na₂CO₃. The mixture was stirred at room temperature for 30 min, then filtered. The filtrate was concentrated and the crude was purified by chromatography on silica gel (CH₂Cl₂:MeOH=100:1˜40:1) to give racemic product (300 mg, yield 72%). Then the solid was separated by chiral HPLC (chiral method B) to give the title compound E53 (84 mg) and E54 (81 mg) as white solids.

E53: LC-MS: 397.2 [M+H]⁺. ¹H NMR (400 MHz, DMSO-d₆): δ 8.40 (s, 1H), 7.83 (s, 1H), 7.06 (d, J=7.6 Hz, 1H), 4.72˜4.40 (m, 2H), 4.28˜4.17 (m, 3H), 4.10˜4.03 (m, 1H), 3.95˜3.90 (m, 2H), 3.44 (t, J=12.0 Hz, 2H), 2.16 (s, 3H), 2.07˜1.87 (m, 2H), 1.77˜1.69 (m, 3H), 1.47˜1.41 (m, 1H). ¹⁹F NMR (376 MHz, DMSO-d₆): δ −217.12. Chiral RT=2.305 min; ee=100%.

E54: LC-MS: 397.2 [M+H]⁺. ¹H NMR (400 MHz, DMSO-d₆): δ 8.40 (s, 1H), 7.83 (s, 1H), 7.06 (d, J=8.0 Hz, 1H), 4.72˜4.40 (m, 2H), 4.28˜4.19 (m, 3H), 4.10˜4.05 (m, 1H), 3.95˜3.90 (m, 2H), 3.44 (t, J=12.0 Hz, 2H), 2.15 (s, 3H), 2.07˜1.85 (m, 2H), 1.77˜1.69 (m, 3H), 1.47˜1.40 (m, 1H). ¹⁹F NMR (376 MHz, DMSO-d₆): δ −217.12. Chiral RT=2.938 min; ee=100%.

### Examples E55, E56, E57 and E58

### Enantiomers 1-4: 14-chloro-4,11-dimethyl-5-{4-[(1R,4R)-2-oxa-5-azabicyclo[2.2.1]heptan-5-yl]cyclohexyl}-8-oxa-2,5,6,12,16,17-hexaazatricyclo[11.3.1.0^{3,7}]heptadeca-1(16),3,6,13(17),14-pentaene (E55-E58)

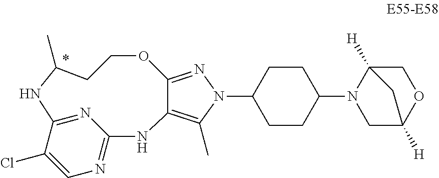 [SMILES: Cc1c2c(nn1C1CCC(N3C[C@H]4C[C@@H]3CO4)CC1)OCCC(C)Nc1nc(ncc1Cl)N2]

To a solution of D94 (200 mg, 0.51 mmol) and (1R,4R)-2-oxa-5-azabicyclo[2.2.1]heptane hydrochloride in MeOH (25 mL) was slowly added NaBH₃CN (97 mg, 1.55 mmol) and the reaction was stirred for 16 hrs. Then sat.NaHCO₃(50 mL) was added and the mixture was stirred for 30 min. MeOH was evaporated and the mixture was filtered, extracted with DCM (3×25 mL). The combined organic layer was washed with sat.NaHCO₃, brine, dried over Na₂SO₄, filtered and concentrated to give the racemic product (200 mg, yield 89.3%). The yellow solid was then further chiral separated (chiral method A) to give the title compounds as white solids.

E55: LC-MS: 474.2[M+H]⁺. ¹H NMR (400 MHz, CDCl₃): δ 7.82 (s, 1H), 6.05 (s, 1H), 5.11 (d, J=4.0 Hz, 1H), 4.48˜4.46 (m, 2H), 4.29 (t, J=8.0 Hz, 1H), 4.08˜4.00 (m, 2H), 3.92˜3.89 (m, 1H), 3.73˜3.71 (m, 1H), 2.68˜2.65 (m, 1H), 2.17 (s, 3H), 2.09˜1.73 (m, 11H), 1.75˜1.46 (m, 4H), 1.31 (d, J=7.2 Hz, 3H). Chiral RT=2.790; ee=100%.

E56: LC-MS: 474.2[M+H]⁺. ¹H NMR (400 MHz, CDCl₃): δ 7.80 (s, 1H), 6.17 (s, 1H), 5.13 (d, J=8.0 Hz, 1H), 4.52˜4.38 (m, 3H), 4.07˜3.95 (m, 3H), 3.71˜3.69 (m, 2H), 3.20˜3.10 (m, 1H), 2.78 (br, 1H), 2.46˜2.23 (m, 2H), 2.17 (s, 3H), 1.92˜1.72 (m, 9H), 1.50˜1.47 (m, 2H), 1.32 (d, J=6.8 Hz, 3H). Chiral RT=3.021; ee=99.1%.

E57: LC-MS: 474.2[M+H]⁺. ¹H NMR (400 MHz, CDCl₃): δ 7.81 (s, 1H), 6.09 (s, 1H), 5.11 (d, J=8.0 Hz, 1H), 4.50˜4.43 (m, 2H), 4.31 (t, J=8.0 Hz, 1H), 4.07˜3.99 (m, 2H), 3.87˜3.3.84 (m, 2H), 3.66 (d, J=8.0 Hz, 1H), 3.19 (br, 1H), 2.56 (br, 1H), 2.17 (s, 3H), 2.14˜2.06 (m, 2H), 2.01˜1.73 (m, 9H), 1.46˜1.37 (m, 2H), 1.31 (d, J=8.0 Hz, 3H). Chiral RT=3.293; ee=100%.

E58: LC-MS: 474.2[M+H]⁺. ¹H NMR (400 MHz, CDCl₃): δ 7.80 (s, 1H), 6.18 (s, 1H), 5.14 (d, J=8.0 Hz, 1H), 4.52˜4.50 (m, 2H), 4.43 (br, 1H), 4.11˜3.93 (m, 3H), 3.66 (d, J=4.0 Hz, 1H), 3.14 (br, 1H), 2.78 (m, 1H), 2.36˜2.24 (m, 3H), 2.17 (s, 3H), 1.92˜1.75 (m, 9H), 1.68˜1.60 (m, 2H), 1.33 (d, J=8.0 Hz, 3H). Chiral RT=3.509; ee=99.7%.

### Examples E59 and E60

### Enantiomers 1-2: trans-14-chloro-5-[3-fluoro-1-(2-methoxyethyl)piperidin-4-yl]-4-methyl-8-oxa-2,5,6,12,16,17-hexaazatricyclo[11.3.1.0^{3,7}]heptadeca 1(16),3,6,13(17),14-pentaene (E59-E60)

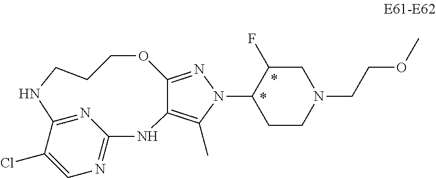 [SMILES: COCCN1CCC(n2nc3c(c2C)Nc2ncc(Cl)c(n2)NCCCO3)C(F)C1]

To a solution of D97 (crude, 0.259 mmol) in MeCN (10.0 mL) were added 1-bromo-2-methoxyethane (72 mg, 0.518 mmol) and K₂CO₃ (359 mg, 2.60 mmol). The reaction was heated to 100° C. and stirred overnight. The cooled mixture was diluted with water and extracted with EtOAc for 3 times. The combined organics were and dried, filtered and concentrated. The crude was purified by column chromatography on silica gel (CH₂Cl₂:MeOH=10:1) (80 mg, yield 70%) and then separated by chiral HPLC (Chiral method A) to give the title compound E59 (16 mg) and E60 (16 mg) as white solids.

E59: LCMS: 440.2[M+H]⁺. ¹H NMR (400 MHz, CDCl₃): δ 7.80 (s, 1H), 6.24 (s, 1H), 5.54 (t, J=6.4 Hz, 1H), 5.08˜4.80 (m, 1H), 4.36 (t, J=4.4 Hz, 2H), 3.91˜3.83 (m, 1H), 3.52 (t, J=5.4 Hz, 4H), 3.40˜3.37 (m, 1H), 3.37 (s, 3H), 3.04˜3.01 (m, 1H), 2.70˜2.64 (m, 2H), 2.47˜2.39 (m, 1H), 2.23˜2.15 (m, 2H), 2.20 (s, 3H), 1.99˜1.74 (m, 3H). ¹⁹F NMR (376 MHz, CDCl₃): δ −187.33. Chiral RT=2.129; ee=100%.

E60: LCMS: 440.2[M+H]⁺. ¹H NMR (400 MHz, CDCl₃): δ 7.81 (s, 1H), 6.15 (s, 1H), 5.53 (d, J=6.2 Hz, 1H), 5.12˜4.77 (m, 1H), 4.36 (t, J=4.6 Hz, 2H), 3.91˜3.84 (m, 1H), 3.52 (t, J=5.4 Hz, 4H), 3.40˜3.37 (m, 1H), 3.37 (s, 3H), 3.02 (d, J=9.6 Hz, 1H), 2.72˜2.63 (m, 2H), 2.45˜2.38 (m, 1H), 2.23˜2.15 (m, 2H), 2.20 (s, 3H), 1.96˜1.80 (m, 3H). ¹⁹F NMR (376 MHz, CDCl₃): δ −187.34. Chiral RT=3.508; ee=100%.

### Examples E61 and E62

### Enantiomers 1-2: trans-14-chloro-5-[3-fluoro-1-(oxetan-3-yl)piperidin-4-yl]-4-methyl-8-oxa-2,5,6,12,16,17-hexaazatricyclo[11.3.1.0^{3,7}]heptadeca-1(16),3,6,13(17),14-pentaene (E61-E62)

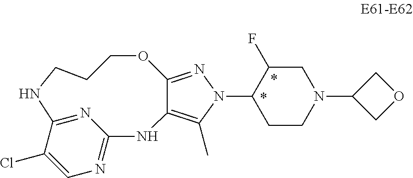 [SMILES: Cc1c2c(nn1C1CCN(C3COC3)CC1F)OCCCNc1nc(ncc1Cl)N2]

To a solution of D97 (500 mg, 1.3 mmol) in CH₃OH (10.0 mL) at 0° C. was added NaBH₃CN (572 g, 9.1 mmol). After stirred at room temperature for 1 hour, oxetan-3-one (468 mg, 6.5 mmol) was added at 0° C. and the reaction was stirred at room temperature overnight. The reaction was quenched with sat.NaHCO₃ and the resultant was extracted with EtOAc for 3 times. The combined organics were dried, filtered and concentrated. The crude was purified by column chromatography on silica gel (CH₂Cl₂:MeOH=10:1)(40 mg, 7%) and then was separated by chiral HPLC (chiral method A) to give the title compounds E61 (17 mg) and E62 (17 mg) as white solids.

E61: LC-MS: 438.3[M+H]⁺. ¹H NMR (400 MHz, CDCl₃): δ 7.81 (s, 1H), 6.17 (s, 1H), 5.54 (s, 1H), 4.94 (d, J=54.2 Hz, 1H), 4.68 (t, J=6.4 Hz, 2H), 4.61 (br, 2H), 4.39 (br, 2H), 3.95˜3.90 (m, 1H), 3.62 (t, J=6.2 Hz, 1H), 3.52 (s, 2H), 3.18˜3.15 (m, 1H), 2.82 (d, J=10.8 Hz, 1H), 2.44˜2.39 (m, 1H), 2.20 (s, 3H), 2.04˜1.91 (m, 5H). ¹⁹F NMR (376 MHz, CDCl₃): δ −187.32. Chiral RT=4.072; ee=100%.

E62: LCMS: 438.3[M+H]⁺. ¹H NMR (400 MHz, CDCl₃): δ 7.81 (s, 1H), 6.24 (s, 1H), 5.55 (t, J=6.0 Hz, 1H), 5.03˜4.80 (m, 1H), 4.68 (t, J=6.6 Hz, 2H), 4.61 (t, J=5.4 Hz, 2H), 4.39 (t, J=4.6 Hz, 2H), 3.95˜3.89 (m, 1H), 3.62 (t, J=6.4 Hz, 1H), 3.52 (br, 2H), 3.18˜3.15 (m, 1H), 2.82 (d, J=10.8 Hz, 1H), 2.44˜2.39 (m, 1H), 2.20 (s, 3H), 2.07˜1.86 (m, 5H). ¹⁹F NMR (376 MHz, CDCl₃): δ −187.32. Chiral RT=6.271 min; ee=100%.

### Examples E63 and E64

### Enantiomers 1-2: 14-Chloro-10-fluoro-4,11-dimethyl-5-(oxan-4-yl)-8-oxa-2,5,6,12, 16,17-hexaazatricyclo[11.3.1.0^{3,7}]heptadeca-1(16),3,6,13(17),14-pentaene (E63-E64)

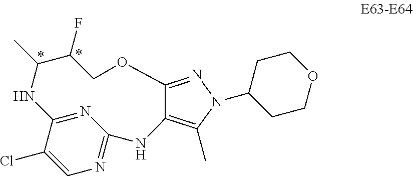 [SMILES: Cc1c2c(nn1C1CCOCC1)OCC(F)C(C)Nc1nc(ncc1Cl)N2]

To a solution of D106 (180 mg, 0.39 mmol) in MeOH (54 mL) were added HOAc (5.4 mL) and Zn (254 mg, 3.89 mmol). The mixture was stirred at 75° C. under nitrogen for 3 hrs. The cooled mixture was filtered and the filtrate was concentrated. The residue was dissolved in DCM (50 mL) the mixture was filtered. The filtrate was washed with sat. NaHCO₃ (50 mL), water (50 mL) and brine (20 mL), dried over Na₂SO₄, concentrated. The crude was purified by pre-TLC (CH₂Cl₂:MeOH=20:1) (120 mg, yield 78%) and further separated by chiral HPLC (Chiral method E) to give the title compounds E63 (15 mg) and E64 (17 mg) as white solids.

E63: LC-MS: 397.2[M+H]⁺. ¹H NMR (400 MHz, CDCl₃): δ 7.82 (s, 1H), 6.76 (s, 1H), 5.23 (m, 1H), 4.67˜4.25 (m, 4H), 4.12˜4.05 (m, 3H), 3.51 (t, J=12.4 Hz, 2H), 2.30˜2.13 (m, 5H), 1.78 (t, J=11.2 Hz, 2H), 1.42 (d, J=7.2 Hz, 3H). Chiral RT=2.397 min; ee=100%.

E64: LC-MS: 397.2[M+H]⁺. ¹H NMR (400 MHz, CDCl₃): δ 7.80 (s, 1H), 7.05 (s, 1H), 5.28 (m, 1H), 4.68˜4.32 (m, 4H), 4.12˜4.05 (m, 3H), 3.51 (t, J=12.4 Hz, 2H), 2.32˜2.16 (m, 5H), 1.78 (t, J=11.2 Hz, 2H), 1.43 (d, J=7.2 Hz, 3H). Chiral RT=3.507 min; ee=100%.

### Example E65

### (11R)-14-chloro-4,11-dimethyl-5-[1-(oxetan-3-yl)piperidin-4-yl]-8-oxa-2,5,6,12,16,17-hexaazatricyclo[11.3.1.0^{3,7}]heptadeca-1(16),3,6,13(17),14-pentaene (E65)

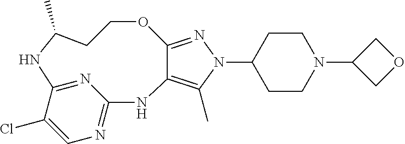 [SMILES: Cc1c2c(nn1C1CCN(C3COC3)CC1)OCC[C@@H](C)Nc1nc(ncc1Cl)N2]

To a solution of D100 (200 mg, 0.53 mmol) and oxetan-3-one (190 mg, 2.65 mmol) in MeOH (20 mL) was added NaBH₃CN (67 mg, 1.55 mmol) and the reaction was stirred for 16 hrs at room temperature. Sat.NaHCO₃(5 mL) was added and the reaction was stirred for 10 min. The mixture was concentrated, filtered and extracted with DCM (50 mL). The combined organic layer was washed with sat.NaHCO₃, brine, dried over Na₂SO₄, filtered and concentrated. The crude was purified by column chromatography on silica gel to give the title compound as a white solid (120 mg, yield 52%). LC-MS: 434.2[M+H]⁺. ¹H NMR (400 MHz, CDCl₃): δ 7.82 (s, 1H), 6.08 (s, 1H), 5.12 (d, J=7.2 Hz, 1H), 4.77˜4.57 (m, 4H), 4.51˜4.27 (m, 2H), 4.02 (d, J=3.2 Hz, 1H), 3.93˜3.77 (m, 1H), 3.52 (t, J=6.4 Hz, 1H), 2.87 (d, J=11.2 Hz, 2H), 2.27 (q, J=12.4 Hz, 2H), 2.19 (s, 3H), 2.04˜1.73 (m, 6H), 1.31 (d, J=7.2 Hz, 3H).

### Example E66

### 14-Bromo-4-methyl-5-(oxetan-3-yl)-8-oxa-2,5,6,12,16,17-hexaazatricyclo[11.3.1.0^{3,7}]heptadeca-1(16),3,6,13(17),14-pentaene (E66)

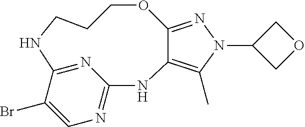 [SMILES: Cc1c2c(nn1C1COC1)OCCCNc1nc(ncc1Br)N2]

To a solution of D108 (133 mg, 0.297 mmol) and Fe (133 mg, 2.38 mmol) in EtOH/H₂O (13 mL/2 mL) was added NH₄Cl (266 mg, 4.97 mmol). The reaction was heated to 100° C. and stirred overnight. Sat.NaHCO₃(5 mL) was added and the mixture was filtered, concentrated. The residue was suspended in CH₂Cl₂ (200 mL) and anhydrous Na₂SO₄ was added. The resultant was stirred at room temperature for 20 min, filtered and the filtrate was concentrated. The residue was slurry in MeCN (10 mL) and the filter cake was dried to give the title compound as a white solid (8 mg, yield 7%). LC-MS: 381.1[M+H]⁺. ¹H NMR (400 MHz, DMSO-d₆): δ 8.38 (s, 1H), 7.84 (s, 1H), 7.18 (t, J=5.6 Hz, 1H), 5.44˜5.37 (m, 1H), 4.89 (t, J=6.2 Hz, 2H), 4.80 (t, J=7.2 Hz, 2H), 4.28 (t, J=4.4 Hz, 2H), 3.29˜3.28 (m, 2H), 2.09 (s, 3H), 1.73 (br, 2H).

### Examples E67 and E68

### Enantiomers 1-2: (11R)-14-chloro-4,11-dimethyl-5-[1-(oxetan-3-yl)piperidin-3-yl]-8-oxa-2,5,6,12,16,17-hexaazatricyclo[11.3.1.0^{3,7}]heptadeca-1(16),3,6,13(17),14-pentaene (E67-E68)

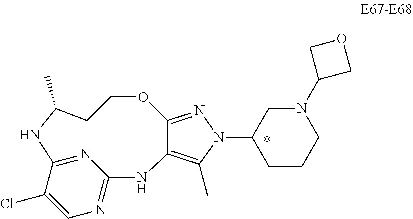 [SMILES: Cc1c2c(nn1C1CCCN(C3COC3)C1)OCC[C@@H](C)Nc1nc(ncc1Cl)N2]

To a solution of D112 (300 mg, 0.80 mmol) and oxetan-3-one (268 mg, 4.0 mmol) in MeOH (30 mL) was slowly added NaBH₃CN (150 mg, 2.4 mmol) and the reaction was stirred at room temperature for 16 hrs. Sat.NaHCO₃ (50 mL) was added and the reaction was stirred for 20 min. Methanol was evaporated and the mixture was filtered, extracted with DCM (3×25 mL). The combined organic layers were washed with sat.NaHCO₃, brine, dried over Na₂SO₄, filtered and concentrated to give a yellow oil (240 mg, yield 69%). The racemic was further chiral separated (chiral method A) to give the title compounds as white solids.

E67: LC-MS: 434.2[M+H]⁺. ¹H NMR (400 MHz, CDCl₃): δ 7.77 (s, 1H), 6.97 (br, 1H), 5.33 (d, J=6.4 Hz, 1H), 4.75˜4.55 (m, 4H), 4.48 (dd, J=2.8, 11.2 Hz, 1H), 4.34˜4.22 (m, 1H), 4.17˜3.95 (m, 2H), 3.56 (t, J=6.4 Hz, 1H), 2.84˜2.69 (m, 2H), 2.34˜2.24 (m, 2H), 2.23 (s, 3H), 2.04˜1.95 (m, 2H), 1.94˜1.81 (m, 4H), 1.34 (d, J=7.2 Hz, 3H). Chiral RT=3.566 min; ee=100%.

E68: LC-MS: 434.2[M+H]⁺. ¹H NMR (400 MHz, CDCl₃): δ 7.81 (s, 1H), 6.20 (s, 1H), 5.12 (d, J=7.2 Hz, 1H), 4.73˜4.57 (m, 4H), 4.48 (dd, J=2.8, 11.2 Hz, 1H), 4.35˜4.23 (m, 1H), 4.17˜3.92 (m, 2H), 3.57 (t, J=6.4 Hz, 1H), 2.96˜2.68 (m, 2H), 2.37 (t, J=10.4 Hz, 1H), 2.20 (s, 3H), 1.97˜1.68 (m, 7H), 1.32 (d, J=7.2 Hz, 3H). Chiral RT=6.693 min; ee=100%.

### Example E69

### 4-Methyl-5-(oxan-4-yl)-8-oxa-2,5,6,12,16,17-hexaazatricyclo[11.3.1.0^{3,7}]heptadeca-1(16),3,6,13(17),14-pentaene-14-carbonitrile (E69)

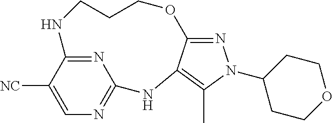 [SMILES: Cc1c2c(nn1C1CCOCC1)OCCCNc1nc(ncc1C#N)N2]

To a solution of D113 (100 mg, 0.33 mmol) in EtOH (40 mL) were added Fe (100 mg) and a solution of NH₄Cl (400 mg) in H₂O (2 mL). The reaction was stirred at 100° C. for 10 hrs. The mixture was filtered and the filter cake was washed with CH₃CN, and purified with prep-HPLC to give the title compound (6 mg, yield 7.5%) as a white solid. LC-MS: 356.2[M+H]⁺. ¹H NMR (400 MHz, DMSO-d₆): δ 8.19 (s, 1H), 4.25˜4.18 (m, 3H), 3.95˜3.91 (m, 2H), 3.47˜3.42 (m, 2H), 3.29˜3.27 (m, 2H), 2.17 (s, 3H), 2.06˜1.91 (m, 2H), 1.73˜1.70 (m, 4H).

### Examples E70-E77

### 14-Chloro-5-(3-fluorooxan-4-yl)-10-methoxy-4-methyl-8-oxa-2,5,6,12,16,17-hexaaza tricyclo[11.3.1.0^{3,7}]heptadeca-1(16),3,6,13(17),14-pentaene (E70-E77)

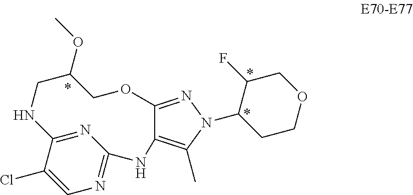 [SMILES: COC1CNc2nc(ncc2Cl)Nc2c(nn(C3CCOCC3F)c2C)OC1]

To a solution of D116 (250 mg, 0.52 mmol) in EtOH (10 mL) were added Fe (250 mg) and a solution of NH₄Cl (1 g) in H₂O (2 mL). The reaction was stirred at 100° C. for 10 hrs. The mixture was filtered. and the filter was concentrated. The crude was purified by pre-TLC to give a yellow solid (180 mg, yield 83%), which was further separated by chiral HPLC (chiral method A and C) to give the title compounds as off-white solids.

E70: 20 mg. LC-MS: 413.2[M+H]⁺. ¹H NMR (400 MHz, CD₃OD): δ 7.74 (s, 1H), 4.78˜4.70 (m, 1H), 4.63˜4.12 (m, 5H), 4.10˜4.00 (m, 1H), 3.83˜3.73 (m, 1H), 3.42 (s, 3H), 3.27˜3.14 (m, 3H), 2.51˜2.30 (m, 2H), 2.22 (s, 3H). ¹⁹F NMR (376 MHz, CD₃OD): δ −232.35 (s, 1F). Chiral RT=3.844 min (chiral method A); ee=100%.

E71: 14 mg. LC-MS: 413.2[M+H]⁺. ¹H NMR (400 MHz, CD₃OD): δ 7.74 (s, 1H), 4.79˜4.72 (m, 1H), 4.63˜4.24 (m, 5H), 4.17 (q, J=7.6 Hz, 1H), 4.05 (q, J=7.6 Hz, 1H), 3.83˜3.72 (m, 1H), 3.42 (s, 3H), 3.27˜3.18 (m, 2H), 2.40˜2.28 (m, 2H), 2.22 (s, 3H). ¹⁹F NMR (376 MHz, CD₃OD): δ −232.26 (s, 1F). Chiral RT=4.09 min (chiral method A), 3.30 min (chiral method C); ee=100%.

E72: 12 mg. LC-MS: 413.2[M+H]⁺. ¹H NMR (400 MHz, CD₃OD): δ 7.74 (s, 1H), 4.76 (td, J=4.8, 9.6 Hz, 1H), 4.43˜4.24 (m, 3H), 4.19 (dd, J=5.2, 10.8 Hz, 1H), 3.98 (d, J=11.6 Hz, 1H), 3.85˜3.74 (m, 1H), 3.54 (t, J=11.2 Hz, 1H), 3.45˜3.36 (m, 4H), 3.27˜3.23 (m, 2H), 2.36˜2.25 (m, 1H), 2.23 (s, 3H), 1.94˜1.91 (m, 1H). ¹⁹F NMR (376 MHz, CD₃OD): δ −195.24 (s, 1F). Chiral RT=4.12 min (chiral method A), 2.63 min (chiral method C); ee=100%.

E73: 12 mg. LC-MS: 413.2[M+H]⁺. ¹H NMR (400 MHz, CD₃OD): δ 7.74 (s, 1H), 4.79˜4.61 (m, 1H), 4.43˜4.23 (m, 3H), 4.17 (dd, J=5.2, 10.8 Hz, 1H), 4.01˜3.98 (m, 1H), 3.83˜3.72 (m, 1H), 3.55 (t, J=11.2 Hz, 1H), 3.46˜3.36 (m, 4H), 3.24 (d, J=13.2 Hz, 2H), 2.36 (dq, J=4.8, 12.6 Hz, 1H), 2.23 (s, 3H), 2.00˜1.97 (d, 1H). ¹⁹F NMR (376 MHz, CD₃OD): δ −195.57 (s, 1F). Chiral purity: RT=4.238 min (chiral method A); ee=100%.

E74: 12 mg. LC-MS: 413.2[M+H]⁺. ¹H NMR (400 MHz, CD₃OD): δ 7.74 (s, 1H), 4.79˜4.70 (m, 1H), 4.62˜4.14 (m, 5H), 4.08˜4.05 (m, 1H), 3.80˜3.77 (m, 1H), 3.42 (s, 3H), 3.26˜3.19 (m, 2H), 2.52˜2.28 (m, 2H), 2.22 (s, 3H). ¹⁹F NMR (376 MHz, CD₃OD): δ −232.35 (s, 1F). Chiral RT=4.637 min (chiral method A); ee=99.4%.

E75: 12 mg. LC-MS: 413.2[M+H]⁺. ¹H NMR (400 MHz, CD₃OD): δ 7.74 (s, 1H), 4.79˜4.71 (m, 1H), 4.63˜4.23 (m, 5H), 4.21˜4.12 (m, 1H), 4.09˜3.99 (m, 1H), 3.84˜3.73 (m, 1H), 3.42 (s, 3H), 3.26˜3.18 (m, 2H), 2.41˜2.27 (m, 2H), 2.22 (s, 3H). ¹⁹F NMR (376 MHz, CD₃OD): δ −232.26 (s, 1F). Chiral RT=4.805 min (chiral method A); ee=96.7%.

E76: 14 mg. LC-MS: 413.2[M+H]⁺. ¹H NMR (400 MHz, CD₃OD): δ 7.74 (s, 1H), 4.77 (dt, J=5.6, 9.6 Hz, 1H), 4.42˜4.25 (m, 3H), 4.19 (dd, J=5.2, 10.8 Hz, 1H), 4.04˜3.93 (m, 1H), 3.86˜3.75 (m, 1H), 3.54 (t, J=11.2 Hz, 1H), 3.46˜3.37 (m, 4H), 3.25 (d, J=14.0 Hz, 2H), 2.35˜2.20 (m, 4H), 1.95˜1.91 (m, 1H). ¹⁹F NMR (376 MHz, CD₃OD): δ −195.24 (s, 1F). Chiral RT=5.314 min (chiral method A); ee=96.7%.

E77: 14 mg. LC-MS: 413.2[M+H]⁺. ¹H NMR (400 MHz, CD₃OD): δ 7.74 (s, 1H), 4.81˜4.60 (m, 1H), 4.42˜4.10 (m, 4H), 4.04˜3.92 (m, 1H), 3.78 (dd, J=2.8, 14.4 Hz, 1H), 3.55 (t, J=11.2 Hz, 1H), 3.47˜3.34 (m, 4H), 3.26˜3.14 (m, 2H), 2.36 (dq, J=5.2, 12.6 Hz, 1H), 2.23 (s, 3H), 2.04˜1.89 (m, 1H). ¹⁹F NMR (376 MHz, CD₃OD): δ −195.57 (s, 1F). Chiral RT=5.658 min (chiral method A); ee=99.2%.

### Examples E78-E85

### Enantiomers 1-8: 14-chloro-4,11-dimethyl-5-(2-methyloxan-4-yl)-8-oxa-2,5,6,12, 16,17-hexaazatricyclo[11.3.1.0^{3,7}]heptadeca-1(16),3,6,13(17),14-pentaene (E78-E85)

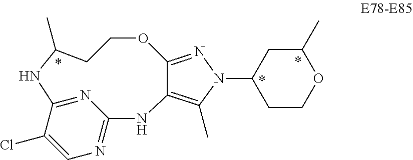 [SMILES: Cc1c2c(nn1C1CCOC(C)C1)OCCC(C)Nc1nc(ncc1Cl)N2]

To a solution of D117 (160 mg) in 95% EtOH (20 mL) were added Fe (160 mg) and NH₄Cl (0.32 g). The reaction was stirred at 100° C. overnight. The reaction mixture was filtered and concentrated. The crude was chiral separated (chiral methods A and B) to give the title compounds as off-white solids.

E78: 3.2 mg, yield 2.3%. LC-MS: 393.3[M+H]⁺. ¹H NMR (400 MHz, CDCl₃): δ 7.77 (s, 1H), 7.00 (br, 1H), 5.34 (d, J=6.0 Hz, 1H), 4.48 (dd, J=3.2, 11.2 Hz, 1H), 4.42˜4.36 (m, 1H), 4.31 (t, J=10.4 Hz, 2H), 4.24˜4.15 (m, 1H), 4.05 (br, 1H), 3.89˜3.74 (m, 1H), 2.21 (s, 3H), 2.10˜1.97 (m, 3H), 1.94˜1.80 (m, 2H), 1.72 (ddd, J=4.8, 9.2, 13.8 Hz, 1H), 1.35 (d, J=6.8 Hz, 3H), 1.21 (d, J=6.0 Hz, 3H). Chiral RT=3.094 min (chiral method A); ee=100%.

E79: 2.6 mg, yield 1.8%. LC-MS: 393.3[M+H]⁺. ¹H NMR (400 MHz, CDCl₃): δ 7.79 (s, 1H), 6.69 (br, 1H), 5.27 (d, J=6.8 Hz, 1H), 4.52˜4.44 (m, 1H), 4.43˜4.27 (m, 3H), 4.26˜4.15 (m, 1H), 4.08˜3.98 (m, 1H), 3.88˜3.79 (m, 1H), 2.20 (s, 3H), 2.03˜1.79 (m, 5H), 1.71 (ddd, J=4.8, 9.2, 13.8 Hz, 1H), 1.34 (d, J=6.8 Hz, 3H), 1.20 (d, J=6.0 Hz, 3H). Chiral RT=3.780 min (chiral method A), 2.157 min (chiral method B); ee=97.5%.

E80: 1 mg, yield 0.7%. LC-MS: 393.3[M+H]⁺. ¹H NMR (400 MHz, CDCl₃): δ 7.67 (s, 1H), 5.80 (d, J=6.0 Hz, 1H), 4.49 (d, J=10.6 Hz, 1H), 4.32˜4.22 (m, 1H), 4.12˜4.09 (m, 3H), 3.59˜3.46 (m, 3H), 2.29 (s, 3H), 2.21˜2.07 (m, 1H), 2.01˜1.82 (m, 4H), 1.73 (d, J=11.6 Hz, 1H), 1.39 (d, J=6.8 Hz, 3H), 1.27 (d, J=6.0 Hz, 3H). Chiral RT=3.780 min (chiral method A), 2.731 min (chiral method B); ee=97.5%.

E81: 3 mg, yield 2.2%. LC-MS: 393.3[M+H]⁺. ¹H NMR (400 MHz, CDCl₃): δ 10.11 (br, 1H), 7.61 (s, 1H), 6.08 (d, J=5.6 Hz, 1H), 4.55˜4.37 (m, 2H), 4.32˜4.08 (m, 4H), 3.88˜3.76 (m, 1H), 2.30 (s, 3H), 2.09˜1.84 (m, 5H), 1.78˜1.68 (m, 1H), 1.43 (d, J=6.8 Hz, 3H), 1.22 (d, J=6.0 Hz, 3H). Chiral RT=3.780 min (chiral method A), 3.306 min (chiral method B); ee=97.5%.

E82: 2.8 mg, yield 2%. LC-MS: 393.3[M+H]⁺. ¹H NMR (400 MHz, CDCl₃): δ 7.78 (s, 1H), 6.98 (br, 1H), 5.34 (d, J=6.0 Hz, 1H), 4.48 (dd, J=2.4, 11.2 Hz, 1H), 4.43˜4.37 (m, 1H), 4.36˜4.28 (m, 2H), 4.25˜4.16 (m, 1H), 4.09˜3.99 (m, 1H), 3.89˜3.80 (m, 1H), 2.21 (s, 3H), 2.07˜1.80 (m, 5H), 1.71 (ddd, J=5.2, 9.2, 13.8 Hz, 1H), 1.35 (d, J=6.8 Hz, 3H), 1.20 (d, J=6.8 Hz, 3H). Chiral RT=4.414 min (chiral method A); ee=100%.

E83: 1.1 mg, yield 0.8%. LC-MS: 393.3[M+H]⁺. ¹H NMR (400 MHz, CDCl₃): δ 7.96 (br, 1H), 7.72 (s, 1H), 5.58 (d, J=5.6 Hz, 1H), 4.54˜4.44 (m, 1H), 4.35˜4.24 (m, 1H), 4.16˜4.00 (m, 3H), 3.61˜3.48 (m, 2H), 2.26 (s, 3H), 2.20˜2.09 (m, 1H), 2.01˜1.82 (m, 4H), 1.73 (d, J=13.2 Hz, 1H), 1.37 (d, J=6.8 Hz, 3H), 1.27 (d, J=6.0 Hz, 3H). Chiral RT=4.840 min (chiral method A); ee=100%.

E84: 1.2 mg, yield 0.8%. LC-MS: 393.3 [M+H]⁺. ¹H NMR (400 MHz, CDCl₃): δ 7.82 (br, 1H), 7.73 (s, 1H), 5.54 (d, J=6.0 Hz, 1H), 4.56˜4.44 (m, 1H), 4.35˜4.23 (m, 1H), 4.09 (t, J=11.6 Hz, 3H), 3.55 (t, J=11.2 Hz, 2H), 2.31˜2.18 (m, 4H), 1.98˜1.71 (m, 5H), 1.36 (d, J=6.8 Hz, 3H), 1.25 (d, J=6.0 Hz, 3H). Chiral RT=5.789 min (chiral method A); ee=100%.

E85: 2.5 mg, yield 1.8%. LC-MS: 393.3 [M+H]⁺. ¹H NMR (400 MHz, CDCl₃): δ 7.97 (s, 1H), 7.68 (s, 1H), 5.80 (d, J=5.6 Hz, 1H), 4.49 (d, J=12.4 Hz, 1H), 4.33˜4.24 (m, 1H), 4.16˜4.03 (m, 3H), 3.55 (t, J=11.2 Hz, 2H), 2.28 (s, 3H), 2.22 (dd, J=4.0, 12.0 Hz, 1H), 2.03˜1.74 (m, 5H), 1.39 (d, J=6.8 Hz, 3H), 1.25 (d, J=6.0 Hz, 3H). Chiral RT=6.285 min (chiral method A); ee=100%.

### Examples E86 and E87

### Enantiomers 1-2: 14-chloro-5-[4-fluorooxan-3-yl]-4-methyl-8-oxa-2,5,6,12,16,17-hexaazatricyclo [11.3.1.0^{3,7}]heptadeca-1(16),3,6,13(17),14-pentaene (E86-E87)

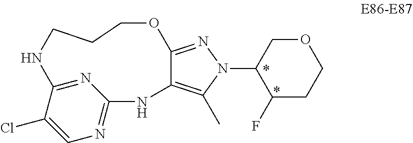 [SMILES: Cc1c2c(nn1C1COCCC1F)OCCCNc1nc(ncc1Cl)N2]

To a solution of D120 (230 mg, 0.512 mmol) in EtOH (27 mL) were added Fe (229 mg, 4.10 mmol) and a solution of NH₄Cl (274 mg, 5.12 mmol) in H₂O (3.0 mL). The reaction was stirred at 110° C. for 16 hrs. The reaction was quenched with aq. Na₂CO₃ and the mixture was filtered through Celite. The filtrate was concentrated and the crude was purified by flash column chromatography on silica gel (CH₂Cl₂:MeOH=50:1 to 25:1) (70 mg, 35.7%) and further separated by chiral HPLC (Chiral method A) to give the title compounds E86 (7 mg) and E87 (9 mg) as white solids.

E86: LC-MS: 383.2[M+H]⁺. ¹H NMR (400 MHz, DMSO-d₆): δ 8.36 (s, 1H), 7.76 (s, 1H), 7.34 (s, 1H), 5.11˜4.97 (m, 1H), 4.20˜4.18 (m, 3H), 3.96˜3.94 (m, 2H), 3.56 (t, J=11.2 Hz, 1H), 3.51˜3.42 (m, 1H), 3.33˜3.28 (m, 2H), 2.16˜2.14 (m, 4H), 1.84˜1.72 (m, 3H). ¹⁹F NMR (376 MHz, DMSO-d₆): δ −177.03. Chiral RT=3.972; ee=100%.

E87: LC-MS: 383.2[M+H]⁺. ¹H NMR (400 MHz, DMSO-d₆): δ 8.36 (s, 1H), 7.77 (s, 1H), 7.34 (s, 1H), 5.11˜4.97 (m, 1H), 4.20˜4.18 (m, 3H), 3.96˜3.94 (m, 2H), 3.56 (t, J=10.8 Hz, 1H), 3.45 (t, J=11.8 Hz, 1H), 3.30˜3.27 (m, 2H), 2.18˜2.08 (m, 4H), 1.84˜1.72 (m, 3H). ¹⁹F NMR (376 MHz, DMSO-d₆): δ −177.03. Chiral RT=5.717; ee=100%.

### Examples E88 and E89

### Enantiomers 1-2: 14-chloro-11-(difluoromethyl)-4-methyl-5-(oxan-4-yl)-8-oxa-2,5,6, 12,16,17-hexaazatricyclo[11.3.1.0^{3,7}]heptadeca-1(16),3,6,13(17),14-pentaene (E88-E89)

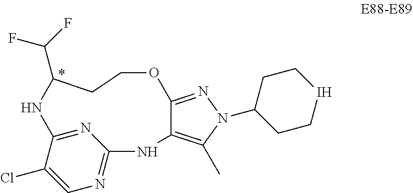

To a suspension of D127 (190 mg, 0.395 mmol) in ethanol (18 mL) was added Fe (110 mg, 1.974 mmol) followed by the addition of ammonia hydrochloride (106 mg, 1.974 mmol) in water (9 mL). The reaction was sealed and heated to 90° C. overnight. After cooled to room temperature, the mixture was filtered and the filtrate was concentrated. The crude was purified by C18 chromatography (5˜95% CH₃CN in water) (99 mg, yield 60.5%) and then separated by chiral HPLC (chiral method A) to give the title compound E88 (13 mg) and E89 (15 mg) as white solids.

E88: LC-MS: 415.2[M+H]⁺. ¹H NMR (400 MHz, DMSO-d₆): δ 8.57 (s, 1H), 7.95 (s, 1H), 7.28 (d, J=8.4 Hz, 1H), 6.30 (dt, J=56.8, 6.0 Hz, 1H), 4.40˜4.16 (m, 4H), 4.02˜3.97 (m, 2H), 3.50 (t, J=11.6 Hz, 2H), 2.22 (s, 3H), 2.16˜1.92 (m, 3H), 1.85˜1.73 (m, 2H), 1.58˜1.52 (m, 1H). ¹⁹F NMR (376 MHz, DMSO-d₆): δ −120.56 (d, J=280.5 Hz, 1F), −127.79 (d, J=280.1 Hz, 1F). Chiral RT=3.472 min; ee=100%.

E89: LC-MS: 415.2[M+H]⁺. ¹H NMR (400 MHz, DMSO-d₆): δ 8.51 (s, 1H), 7.89 (s, 1H), 7.22 (d, J=8.4 Hz, 1H), 6.24 (dt, J=56.8, 6.0 Hz, 1H), 4.34˜4.10 (m, 4H), 3.95˜3.91 (m, 2H), 3.43 (t, J=11.8 Hz, 2H), 2.16 (s, 3H), 2.09˜1.85 (m, 3H), 1.77˜1.66 (m, 2H), 1.52˜1.46 (m, 1H). ¹⁹F NMR (376 MHz, DMSO-d₆): δ −120.55 (d, J=280.1 Hz, 1F), −127.79 (d, J=280.1 Hz, 1F). Chiral RT=10.500 min; ee=100%.

### Examples E90 and E91

### Isomers 1-2:4-{14-chloro-4-methyl-8-oxa-2,5,6,12,16,17-hexaazatricyclo[11.3.1.0^{3,7}]heptadeca-1(16),3,6,13(17),14-pentaen-5-yl}cyclohexan-1-ol (E90-E91)

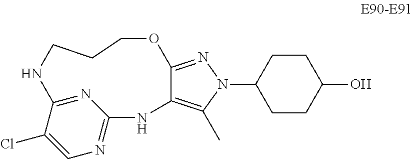 [SMILES: Cc1c2c(nn1C1CCC(O)CC1)OCCCNc1nc(ncc1Cl)N2]

To a solution of D85 (200 mg, 0.531 mmol) in MeOH (5 mL) at 0° C. was added NaBH₄ (6 mg, 0.159 mmol). The reaction was stirred at room temperature for 45 minutes. Water (10 mL) was added and the mixture was extracted with CH₂Cl₂ (3×15 mL). The combined organics were washed with brine (20 mL), dried over anhydrous Na₂SO₄, filtered and concentrated. The crude was purified by prep-TLC (CH₂Cl₂:MeOH=10:1)(120 mg, yield 60%) and then separated by chiral HPLC (chiral method E) to give the title compound E90 (58 mg) and E91 (12 mg) as white solids.

E90: LC-MS: 379.3 [M+H]⁺. ¹H NMR (400 MHz, CDCl₃): δ 7.82 (s, 1H), 6.09 (s, 1H), 5.53 (t, J=6.0 Hz, 1H), 4.42˜4.35 (m, 2H), 3.93˜3.83 (m, 1H), 3.80˜3.69 (m, 1H), 3.56˜3.47 (m, 2H), 2.20 (s, 3H), 2.14˜2.11 (m, 2H), 2.09˜1.98 (m, 2H), 1.94˜1.91 (m, 4H), 1.49˜1.33 (m, 2H). Chiral RT=4.155.

E91: LCMS: 379.3 [M+H]⁺. ¹H NMR (400 MHz, CDCl₃): δ 7.81 (s, 1H), 6.06 (s, 1H), 5.53 (t, J=6.0 Hz, 1H), 4.41˜4.38 (m, 2H), 4.08 (br, 1H), 3.92˜3.86 (m, 1H), 3.54˜3.49 (m, 2H), 2.36˜2.24 (m, 2H), 2.22 (s, 3H), 2.03˜1.89 (m, 4H), 1.73˜1.65 (m, 4H). Chiral RT=5.923.

### Examples E92-E95

### Enantiomers 1-4: 4-{14-chloro-4,11-dimethyl-8-oxa-2,5,6,12,16,17-hexaazatricyclo [11.3.1.0^{3,7}]heptadeca-1(16),3,6,13(17),14-pentaen-5-yl}cyclohexan-1-ol (E92-E95)

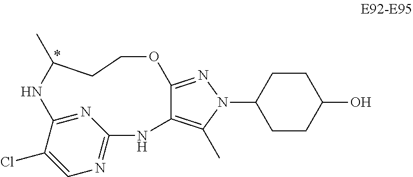 [SMILES: Cc1c2c(nn1C1CCC(O)CC1)OCCC(C)Nc1nc(ncc1Cl)N2]

To a solution of D94 (250 mg, 0.64 mmol) in MeOH (30 mL) was added NaBH₄ (48.4 mg, 1.3 mmol) at 0˜5° C. The reaction was stirred at room temperature for 2 hrs. The mixture was poured into aq. NaHCO₃(30 mL) and extracted with EtOAc (2×80 mL). The combined organic layer was washed with brine, dried over anhydrous Na₂SO₄ and concentrated in vacuo to give the racemic product. The white solid was further chiral separated by chiral HPLC (chiral method B) to give the title compounds as white solids.

E92: 1.1 mg yield 0.4%. LC-MS: 393.3[M+H]⁺. ¹H NMR (400 MHz, CDCl₃): δ 9.6 (s, 1H), 7.63 (s, 1H), 5.96 (m, 1H), 4.47 (d, J=13.6 Hz, 1H), 4.29 (d, J=9.2 Hz, 1H), 4.09 (m, 2H), 2.29 (m, 5H), 1.92 (m, 4H), 1.66 (m, 4H), 1.25 (d, J=6.8 Hz, 3H). Chiral RT=2.656 min; ee=100%.

E93: 6 mg yield 2.4%. LC-MS: 393.3[M+H]⁺. ¹H NMR (400 MHz, CDCl₃): δ 8.98 (br, 1H), 7.68 (s, 1H), 5.81 (d, J=6.0 Hz, 1H), 4.53˜4.43 (m, 1H), 4.30˜4.17 (m, 1H), 4.10 (dd, J=3.6, 6.4 Hz, 1H), 3.95˜3.67 (m, 2H), 2.27 (s, 3H), 2.15˜1.81 (m, 8H), 1.51˜1.41 (m, 2H), 1.38 (d, J=6.8 Hz, 3H). Chiral RT=2.824 min; ee=97.3%.

E94: 1.1 mg yield 0.4%. LC-MS: 393.3[M+H]⁺. ¹H NMR (400 MHz, CDCl₃): 7.67 (s, 1H), 5.85˜5.61 (m, 1H), 4.48 (d, J=12.0 Hz, 1H), 4.29 (d, J=9.2 Hz, 1H), 4.09 (br, 2H), 2.36˜2.22 (m, 5H), 2.04˜1.82 (m, 4H), 1.68 (d, J=12.0 Hz, 4H), 1.38 (d, J=6.8 Hz, 3H). Chiral RT=3.180 min; ee=97.5%.

E95: 6 mg yield 2.4%. LC-MS: 393.3[M+H]⁺. ¹H NMR (400 MHz, CDCl₃): δ 7.85 (br, 1H), 7.73 (s, 1H), 5.54 (d, J=6.0 Hz, 1H), 4.55˜4.41 (m, 1H), 4.35˜4.19 (m, 1H), 4.07 (d, J=3.6 Hz, 1H), 3.95˜3.67 (m, 2H), 2.24 (s, 3H), 2.15˜1.84 (m, 8H), 1.51˜1.39 (m, 2H), 1.36 (d, J=6.8 Hz, 3H). Chiral RT=3.37 min; ee=100%.

### Examples E96-E99

### Enantiomers 1-4: 14-chloro-5-[3-fluoro-1-(oxetan-3-yl)piperidin-4-yl]-4,11-dimethyl-8-oxa-2,5,6,12,16,17-hexaazatricyclo[11.3.1.0^{3,7}]heptadeca-1(16),3,6,13(17),14-pentaene (E96-E99)

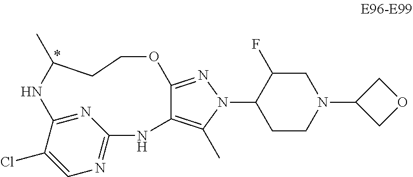 [SMILES: Cc1c2c(nn1C1CCN(C3COC3)CC1F)OCCC(C)Nc1nc(ncc1Cl)N2]

A solution of D130 (151 mg, 0.38 mmol) and oxetan-3-one (124 mg, 1.717 mmol) in MeOH (20 mL) was stirred at room temperature for 1 hour and then NaBH₃(CN) (108 mg, 1.717 mmol) was added. The reaction was stirred at room temperature overnight. Solvent was evaporated and the crude was purified by pre-HPLC followed by chiral separating (chiral method C) to give the title compounds as white solids.

E96: LC-MS: 452.2[M+H]⁺. ¹H NMR (400 MHz, CDCl₃): δ 8.14 (br, 1H), 7.71 (s, 1H), 5.61 (br, 1H), 5.11˜4.85 (m, 1H), 4.74˜4.64 (m, 2H), 4.60 (br, 2H), 4.47 (d, J=12.0 Hz, 1H), 4.35˜4.25 (m, 1H), 4.09 (br, 1H), 3.92 (d, J=13.8 Hz, 1H), 3.63 (t, J=6.4 Hz, 1H), 3.25˜3.09 (m, 1H), 2.82 (d, J=10.4 Hz, 1H), 2.50˜2.32 (m, 1H), 2.25 (s, 3H), 2.11˜1.89 (m, 5H), 1.38 (d, J=6.8 Hz, 3H). ¹⁹F NMR (376 MHz, CDCl₃): δ −187.108 (s, 1F). Chiral RT=2.748 min; ee=98.8%.

E97: LC-MS: 452.2[M+H]⁺. ¹H NMR (400 MHz, CDCl₃): δ 7.73 (s, 1H), 7.59 (br, 1H), 5.48 (d, J=6.8 Hz, 1H), 5.12˜4.83 (m, 1H), 4.72˜4.66 (m, 2H), 4.64˜4.57 (m, 2H), 4.51˜4.43 (m, 1H), 4.36˜4.26 (m, 1H), 4.07 (d, J=3.2 Hz, 1H), 3.97˜3.85 (m, 1H), 3.62 (q, J=6.4 Hz, 1H), 3.23˜3.12 (m, 1H), 2.82 (d, J=10.0 Hz, 1H), 2.46˜2.32 (m, 1H), 2.23 (s, 3H), 2.09˜1.86 (m, 5H), 1.36 (d, J=6.8 Hz, 3H). ¹⁹F NMR (376 MHz, CDCl₃): δ −187.043 (s, 1F). Chiral RT=2.812 min; ee=98.1%.

E98: LC-MS: 452.2[M+H]⁺. ¹H NMR (400 MHz, CDCl₃): δ 7.81 (s, 1H), 6.47 (br, 1H), 5.21 (d, J=6.8 Hz, 1H), 5.01˜4.79 (m, 1H), 4.71˜4.65 (m, 2H), 4.63˜4.57 (m, 2H), 4.46 (dd, J=2.4, 11.2 Hz, 1H), 4.39˜4.28 (m, 1H), 4.12˜3.83 (m, 2H), 3.62 (quin, J=6.4 Hz, 1H), 3.19˜3.12 (m, 1H), 2.83 (d, J=9.6 Hz, 1H), 2.53˜2.38 (m, 1H), 2.21 (s, 3H), 2.08˜1.77 (m, 5H), 1.32 (d, J=6.8 Hz, 3H). ¹⁹F NMR (376 MHz, CDCl₃): δ −187.647 (s, 1F). Chiral RT=2.955 min; ee=100%.

E99: LC-MS: 452.2[M+H]⁺. ¹H NMR (400 MHz, CDCl₃): δ 8.14 (br, 1H), 7.71 (s, 1H), 5.61 (br, 1H), 5.10˜4.86 (m, 1H), 4.74˜4.66 (m, 2H), 4.60 (br, 2H), 4.47 (d, J=12.0 Hz, 1H), 4.35˜4.24 (m, 1H), 4.09 (br, 1H), 3.92 (d, J=13.6 Hz, 1H), 3.63 (t, J=6.4 Hz, 1H), 3.22˜3.14 (m, 1H), 2.82 (d, J=10.4 Hz, 1H), 2.46˜2.32 (m, 1H), 2.25 (s, 3H), 2.10˜1.85 (m, 5H), 1.38 (d, J=6.8 Hz, 3H). ¹⁹F NMR (376 MHz, CDCl₃): δ −187.578 (s, 1F). Chiral RT=3.255 min; ee=98.1%.

### Examples E100 and E101

### Enantiomers 1-2: (11R)-14-chloro-5-[3-fluoro-1-(2-methoxyethyl)piperidin-4-yl]-4,11-dimethyl-8-oxa-2,5,6,12,16,17-hexaazatricyclo[11.3.1.0^{3,7}]heptadeca-1(16),3,6,13(17),14-pentaene (E100-E101)

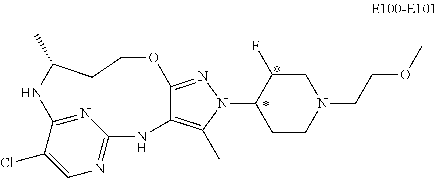 [SMILES: COCCN1CCC(n2nc3c(c2C)Nc2ncc(Cl)c(n2)N[C@H](C)CCO3)C(F)C1]

A solution of D133 (396 mg, 1.0 mmol), K₂CO₃ (692 mg, 5.0 mmol) and 1-bromo-2-methoxyethane (209 mg, 1.5 mmol) in CH₃CN (15 mL) was stirred overnight at 60° C. The mixture was extracted with EtOAc (3×30 mL). The combined organic layer was washed with brine, dried over anhydrous Na₂SO₄ and concentrated to give the racemic product (110 mg, yield 25%). The crude was purified by pre-HPLC and further separated by SFC (chiral method A) to give the title compounds as white solids.

E100: LC-MS: 454.2[M+H]⁺. ¹H NMR (400 MHz, CDCl₃): δ 7.76 (s, 1H), 6.95 (br, 1H), 5.32 (d, J=6.8 Hz, 1H), 5.11˜4.83 (m, 1H), 4.44 (dd, J=2.4, 11.2 Hz, 1H), 4.35˜4.22 (m, 1H), 4.05 (br, 1H), 3.95˜3.82 (m, 1H), 3.64 (d, J=6.0 Hz, 2H), 3.53 (t, J=5.2 Hz, 2H), 3.45˜3.37 (m, 1H), 3.37 (s, 3H), 3.03 (d, J=11.2 Hz, 1H), 2.77˜2.57 (m, 2H), 2.50˜2.33 (m, 1H), 2.27˜2.12 (m, 4H), 1.98˜1.68 (m, 2H), 1.34 (d, J=7.0 Hz, 3H). ¹⁹F NMR (376 MHz, CDCl₃): 5˜186.91 (s, 1F). Chiral RT=3.734 min; ee=100%.

E101: LC-MS: 454.2[M+H]⁺. ¹H NMR (400 MHz, CDCl₃): δ 7.73 (s, 1H), 5.52 (d, J=5.6 Hz, 1H), 5.05˜4.69 (m, 1H), 4.43 (d, J=12.0 Hz, 1H), 4.35˜4.23 (m, 1H), 4.05 (br, 1H), 3.89 (d, J=9.2 Hz, 1H), 3.70˜3.48 (m, 4H), 3.45˜3.37 (m, 1H), 3.37 (s, 3H), 3.07 (d, J=11.2 Hz, 1H), 2.72 (d, J=5.6 Hz, 2H), 2.53˜2.37 (m, 1H), 2.24 (m, 4H), 1.97˜1.84 (m, 2H), 1.35 (d, J=6.8 Hz, 3H). ¹⁹F NMR (376 MHz, CDCl₃): δ −187.553 (s, 1F). Chiral RT=5.943 min; ee=100%.

### Examples E102-E105

### Enantiomers 1-4: 14-chloro-4,11-dimethyl-5-{4-[(1S,4S)-2-oxa-5-azabicyclo[2.2.1]heptan-5-yl]cyclohexyl}-8-oxa-2,5,6,12,16,17-hexaazatricyclo-[11.3.1.0^{3,7}]-hepta deca-1(16),3,6,13(17),14-pentaene (E102-105)

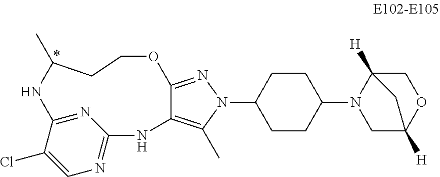 [SMILES: Cc1c2c(nn1C1CCC(N3C[C@@H]4C[C@H]3CO4)CC1)OCCC(C)Nc1nc(ncc1Cl)N2]

To a solution of D94 (200 mg, 0.51 mmol) and (1S,4S)-2-oxa-5-azabicyclo[2.2.1]heptane hydrochloride (346 mg, 2.56 mmol) in MeOH (25 mL) was slowly added NaBH₃CN (97 mg, 1.55 mmol) and the reaction was stirred for 16 hrs. Sat. NaHCO₃(50 mL) was added and the mixture was stirred for 30 min. MeOH was evaporated and the mixture was filtered and extracted with DCM (3×25 mL). The combined organic layer was washed with brine, dried over Na₂SO₄, filtered and concentrated. The crude was further chiral separated (chiral method A) to give the title compounds as yellow solids.

E102: LC-MS: 474.2[M+H]⁺. ¹H NMR (400 MHz, CDCl₃): δ 7.84 (s, 1H), 6.05 (br, 1H), 5.11 (d, J=6.8 Hz, 1H), 4.56˜4.25 (m, 3H), 4.05 (d, J=7.6 Hz, 2H), 3.91˜3.53 (m, 3H), 3.11 (d, J=9.6 Hz, 1H), 2.47 (d, J=10.2 Hz, 2H), 2.18 (s, 3H), 2.13˜1.72 (m, 11H), 1.32 (d, J=6.8 Hz, 3H). Chiral RT=2.841 min; ee=100%.

E103: LC-MS: 474.2[M+H]⁺. ¹H NMR (400 MHz, CDCl₃): δ 7.81 (s, 1H), 6.06 (br, 1H), 5.11 (d, J=6.8 Hz, 1H), 4.58˜4.22 (m, 3H), 4.13˜3.81 (m, 3H), 3.72˜3.52 (m, 2H), 3.05 (d, J=9.6 Hz, 1H), 2.72 (br, 1H), 2.47˜2.20 (m, 3H), 2.20 (s, 3H), 2.04˜1.47 (m, 10H), 1.32 (d, J=6.8 Hz, 3H) Chiral RT=3.031 min; ee=99.7%.

E104: LC-MS: 474.3[M+H]⁺. ¹H NMR (400 MHz, CDCl₃): δ 7.82 (s, 1H), 6.05 (br, 1H), 5.11 (d, J=6.4 Hz, 1H), 4.58˜4.25 (m, 3H), 4.05 (d, J=8.0 Hz, 2H), 3.90˜3.53 (m, 3H), 3.10 (d, J=9.6 Hz, 1H), 2.47 (d, J=9.6 Hz, 2H), 2.18 (s, 3H), 2.11˜1.74 (m, 11H), 1.31 (d, J=6.8 Hz, 3H). Chiral RT=3.296 min; ee=97.5%

E105: LC-MS: 474.2[M+H]⁺. ¹H NMR (400 MHz, CDCl₃): δ 7.81 (s, 1H), 6.08 (br, 1H), 5.11 (d, J=6.8 Hz, 1H), 4.61˜4.20 (m, 3H), 4.11˜3.80 (m, 3H), 3.70˜3.56 (m, 2H), 3.09 (d, J=9.6 Hz, 1H), 2.73 (br, 1H), 2.44˜2.21 (m, 3H), 2.19 (s, 3H), 1.99˜1.71 (m, 6H), 1.68˜1.48 (m, 4H), 1.31 (d, J=6.8 Hz, 3H). Chiral RT=3.539 min; ee=99.3%

### Example E106

### (11R)-14-chloro-4,11-dimethyl-5-[3-(morpholin-4-yl)cyclobutyl]-8-oxa-2,5,6,12,16,17-hexaazatricyclo[11.3.1.0^{3,7}]heptadeca-1(16),3,6,13(17),14-pentaene (E106)

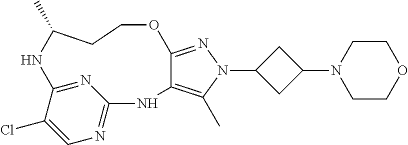 [SMILES: Cc1c2c(nn1C1CC(N3CCOCC3)C1)OCC[C@@H](C)Nc1nc(ncc1Cl)N2]

To a solution of D135 (100 mg, 0.3 mmol) in MeOH (20 mL) was added morpholine (130.7 mg, 1.5 mmol) and AcOH (2 mL). After stirred at room temperature for 30 min, NaBH₃CN (94.26 mg, 1.5 mmol) was added. The reaction was stirred overnight at room temperature. The mixture was poured into the sat.NaCl (aq) and extracted with EtOAc (3×100 mL). The combined organic layer was washed with brine, dried over anhydrous Na₂SO₄ and concentrated in vacuo. The crude was purified by SFC to give the target product as an off-white solid (25 mg, yield 19%). LC-MS: 434.3 [M+H]⁺. ¹H NMR (400 MHz, CD₃OD): δ7.72 (s, 1H), 4.50˜4.37 (m, 3H), 3.97˜3.94 (m, 1H), 3.75˜3.73 (m, 4H), 2.73 (br, 1H), 2.60˜2.47 (m, 8H), 2.17 (s, 3H), 1.80˜1.76 (m, 2H), 1.30˜1.23 (m, 3H).

### Example E107

### (11R)-14-chloro-4,11-dimethyl-5-[1-(oxetan-3-yl)azetidin-3-yl]-8-oxa-2,5,6,12,16,17-hexaazatricyclo[11.3.1.0^{3,7}]heptadeca-1(16),3,6,13(17),14-pentaene (E107)

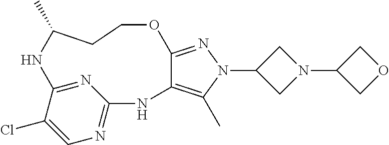 [SMILES: Cc1c2c(nn1C1CN(C3COC3)C1)OCC[C@@H](C)Nc1nc(ncc1Cl)N2]

To a solution of D160 (crude in MeOH (5 mL)) at room temperature were added oxetan-3-one (130 mg, 1.8 mmol), NaBH₃CN (189 mg, 3 mmol) and HOAc (1 mL). The reaction was stirred under argon at 70° C. for 1 hour. The cooled mixture was diluted with water and then concentrated. The crude was purified by prep-HPLC to give the title compound as a white solid (53 mg, yield 22%). LC-MS: 406.1 [M+H]⁺. ¹H NMR (400 MHz, CD₃OD): δ 7.95 (s, 1H), 5.41˜5.35 (m, 1H), 5.01˜4.93 (m, 2H), 4.78˜4.68 (m, 4H), 4.65˜4.61 (m, 2H), 4.55˜4.52 (m, 1H), 4.49˜4.43 (m, 2H), 4.08 (br, 1H), 2.24 (s, 3H), 1.97˜1.83 (m, 2H), 1.39 (s, 3H).

Examples (E108-E378) shown in Table 1 were prepared generally following the above-described Examples.

TABLE 1

| | |
|---|---|
| 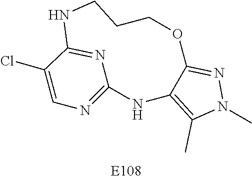 [SMILES: Cc1c2c(nn1C)OCCCNc1nc(ncc1Cl)N2] | 14-Chloro-4,5-dimethyl-8-oxa-2,5,6,12,16,17-hexaazatricyclo [11.3.1.0^{3,7}]heptadeca-1(16),3,6,13(17),14-pentaene (E108) Compound was prepared using procedures similar to those used for preparing E1, starting from 1,5-dimethyl-1H-pyrazole. LC-MS: 295.2 [M + H]⁺. ¹H NMR (400 MHz, CDCl₃): δ 7.82 (s, 1H), 6.12 (s, 1H), 5.56 (s, 1H), 4.39 (t, J = 4.4 Hz, 2H), 3.64 (s, 3H), 3.52 (t, J = 4.8 Hz, 2H), 2.19 (s, 3H), 1.93 (s, 2H). |
| 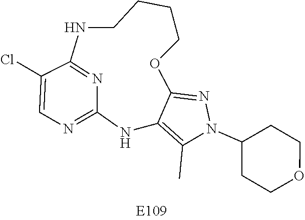 [SMILES: Cc1c2c(nn1C1CCOCC1)OCCCCNc1nc(ncc1Cl)N2] | 15-Chloro-4-methyl-5-(oxan-4-yl)-8-oxa-2,5,6,13,17,18-hexaaza tricyclo[12.3.1.0^{3,7}]octadeca-1(17),3,6,14(18),15-pentaene (E109) Compound was prepared using procedures similar to those used for preparing E1, starting from 4-((tert-butoxycarbonyl)amino)butyl methanesulfonate. LC-MS: 379.5 [M + H]⁺. ¹H NMR (400 MHz, CDCl₃): δ 7.77 (s, 1H), 6.54~6.48 (m, 1H), 5.45 (s, 1H), 4.34 (s, 2H), 4.12~4.09 (m, 3H), 3.50~3.44 (m, 4H), 2.30~2.20 (m, 5H), 1.79~1.75 (m, 6H). |
| 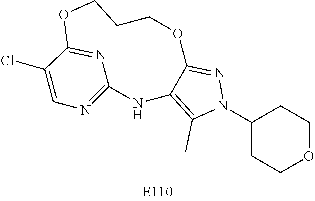 [SMILES: Cc1c2c(nn1C1CCOCC1)OCCCOc1nc(ncc1Cl)N2] | 14-Chloro-4-methyl-5-(oxan-4-yl)-8,12-dioxa-2,5,6,16,17- pentaazatricyclo[11.3.1.0^{3,7}]heptadeca-1(16),3,6,13(17),14- pentaene (E110) Compound was prepared using procedures similar to those used for preparing E5, starting from propane-1,3-diol. LC-MS: 366.2 [M + H]⁺. ¹H NMR (400 MHz, CDCl₃): δ 8.04 (s, 1H), 6.38 (s, 1H), 4.59~4.56 (m, 2H), 4.44~4.41 (m, 2H), 4.13~4.05 (m, 3H), 3.54~3.48 (m, 2H), 2.32~2.22 (m, 5H), 2.06~2.04 (m, 2H), 1.80~1.77 (m, 2H). |
| 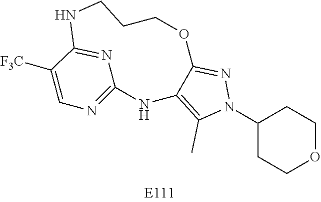 [SMILES: Cc1c2c(nn1C1CCOCC1)OCCCNc1nc(ncc1C(F)(F)F)N2] | 4-Methyl-5-(oxan-4-yl)-14-(trifluoromethyl)-8-oxa-2,5,6,12,16, 17-hexaazatricyclo[11.3.1.0^{3,7}]heptadeca- 1(16),3,6,13(17),14- pentaene (E111) Compound was prepared using procedures similar to those used for preparing E1, starting from 2,4-dichloro-5- (trifluoromethyl)pyrimidine. LC-MS: 399.8 [M + H]⁺. ¹H NMR (400 MHz, CDCl₃): δ 8.07 (s, 1H), 6.69 (s, 1H), 5.56 (s, 1H), 4.41~4.39 (m, 2H), 4.11~4.09 (m, 3H), 3.54~3.48 (m, 4H), 2.29~2.22 (m, 5H), 1.93 (s, 2H), 1.80~1.77 (m, 2H). |
| 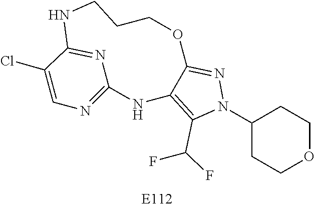 [SMILES: FC(F)c1c2c(nn1C1CCOCC1)OCCCNc1nc(ncc1Cl)N2] | 4-Chloro-4-(difluoromethyl)-5-(oxan-4-yl)-8-oxa-2,5,6,12,16,17- hexaazatricyclo[11.3.1.0^{3,7}]heptadeca-1(16),3,6,13(17),14- pentaene (E112) Compound was prepared using procedures similar to those used for preparing E5, starting from D145. LC-MS: 401.2 [M + H]⁺. ¹H NMR (400 MHz, CDCl₃): δ 7.82 (s, 1H), 7.11 (s, 1H), 6.87~6.61 (m, 1H), 5.61~5.58 (m, 1H), 4.41~4.31 (m, 3H), 4.12~4.08 (m, 2H), 3.54~3.49 (m, 4H), 2.32~2.28 (m, 2H), 1.94~1.87 (m, 4H). ¹⁹FNMR (400 MHz, CDCl₃): δ 112.03 (s, 2F). |
| 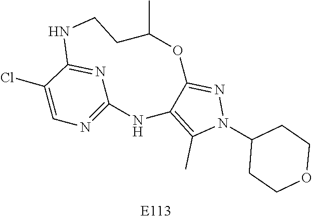 [SMILES: Cc1c2c(nn1C1CCOCC1)OC(C)CCNc1nc(ncc1Cl)N2] | 14-Chloro-4,9-dimethyl-5-(oxan-4-yl)-8-oxa-2,5,6,12,16,17-hexa azatricyclo[11.3.1.0^{3,7}]heptadeca-1(16),3,6,13(17),14-pentaene (E113) Compound was prepared using procedures similar to those used for preparing E1, starting from D5 and 4-((tert- butoxycarbonyl)amino)butan-2-ylmethane-sulfonate. LC-MS: 379.2 [M + H]⁺. ¹H NMR (400 MHz, CDCl₃): δ 7.80 (s, 1H), 6.11 (s, 1H), 5.51 (s, 1H), 4.59~4.56 (m, 1H), 4.11~4.02 (m, 3H), 3.74~ 3.69 (m, 1H), 3.53~3.48 (m, 2H), 3.40~3.33 (m, 1H), 2.35~2.20 (m, 6H), 2.06~2.00 (m, 1H), 1.81~1.69 (m, 2H), 1.51~1.47 (m, 3H). |
| 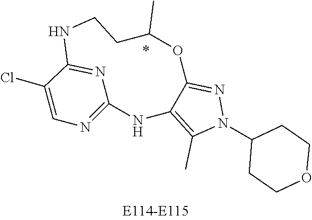 [SMILES: Cc1c2c(nn1C1CCOCC1)OC(C)CCNc1nc(ncc1Cl)N2] | Enantiomers 1-2: 14-chloro-4,9-dimethyl-5-(oxan-4-yl)-8-oxa- 2,5,6,12,16,17-hexaazatricyclo[11.3.1.0^{3,7}]heptadeca- 1(16),3,6,13(17),14-pentaene (E114-E115) Compounds were prepared from chiral separation of E113. E114: LCMS: 379.2 [M + H]⁺. ¹H NMR (400 MHz, CDCl₃): δ 7.80 (s, 1H), 6.16 (br, 1H), 5.50 (br, 1H), 4.60~4.56 (m, 1H), 4.12~4.03 (m, 3H), 3.75~3.69 (m, 1H), 3.53~3.50 (m, 2H), 3.40~3.33 (m, 1H), 2.29~2.23 (m, 2H), 2.23 (s, 3H), 2.06~2.02 (m, 1H), 1.90~1.86 (m, 2H), 1.43 (d, J = 8.0 Hz, 3H), 1.26~1.23 (m, 1H), Chiral RT = 2.049 min (chiral method A); ee = 100%. E115: LCMS: 379.2 [M + H]⁺. ¹H NMR (400 MHz, CDCl₃): δ 7.80 (s, 1H), 6.08 (brs, 1H), 5.50 (brs, 1H), 4.60~4.56 (m, 1H), 4.12~4.03 (m, 3H), 3.75~3.70 (m, 1H), 3.53~3.50 (m, 2H), 3.38~3.37 (m, 1H), 2.43~2.23 (m, 2H), 2.23 (s, 3H), 2.06~2.02 (m, 1H), 1.79~1.75 (m, 2H), 1.43 (d, J = 8.0, 3H), 1.28~1.22 (m, 1H). Chiral RT = 6.673 min |
| | (chiral method A); ee = 100%. |
| 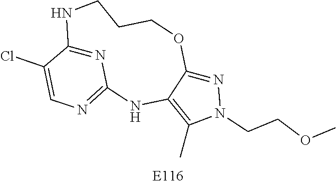 [SMILES: COCCn1nc2c(c1C)Nc1ncc(Cl)c(n1)NCCCO2] | 14-Chloro-5-(2-methoxyethyl)-4-methyl-8-oxa-2,5,6,12,16,17- hexaazatricyclo[11.3.1.0^{3,7}]heptadeca-1(16),3,6,13(17),14- pentaene (E116) Compound was prepared using procedures similar to those used for preparing E8, starting from 1-bromo-2-methoxyethane. LC-MS: 339.2 [M + H]⁺. ¹H NMR (400 MHz, CDCl₃): δ 7.81 (s, 1H), 6.22 (br, 1H), 5.55 (br, 1H), 4.39 (br, 2H), 4.06~4.06 (m, 2H), 3.70- 3.67 (m, 2H), 3.53~3.52 (m, 2H), 3.31 (s, 3H), 2.21 (s, 3H), 1.92 (br, 2H). |
| 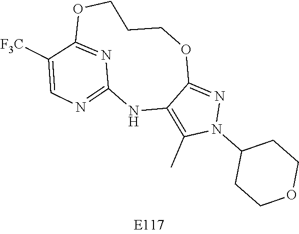 [SMILES: Cc1c2c(nn1C1CCOCC1)OCCCOc1nc(ncc1C(F)(F)F)N2] | 4-Methyl-5-(oxan-4-yl)-14-(trifluoromethyl)-8,12-dioxa-2,5,6, 16,17-pentaazatricyclo[11.3.1.0^{3,7}]heptadeca-1(16),3,6,13(17),14- pentaene (E117) Compound was prepared using procedures similar to those used for preparing E1, starting from 3-bromopropan-1-ol and 2,4-dichloro-5- (trifluoromethyl) pyrimidine. LC-MS: 400.2 [M + H]⁺. ¹H NMR (400 MHz, CDCl₃): δ 8.27 (s, 1H), 7.21 (s, 1H), 4.62~4.59 (m, 2H), 4.44~4.42 (m, 2H), 4.13~4.06 (m, 3H), 3.54-3.48 (m, 2H), 2.33~2.24 (m, 5H), 2.09~2.01 (m, 2H), 1.80~ 1.78 (m, 2H). ¹⁹F NMR (400 MHz, CDCl₃): δ −61.557 (s, 3F). |
| 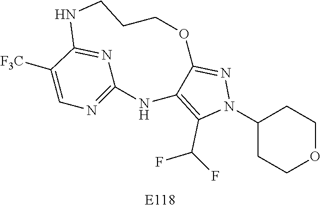 [SMILES: FC(F)c1c2c(nn1C1CCOCC1)OCCCNc1nc(ncc1C(F)(F)F)N2] | 4-(Difluoromethyl)-5-(oxan-4-yl)-14-(trifluoromethyl)-8-oxa- 2,5,6,12,16,17-hexaazatricyclo[11.3.1.0^{3,7}]heptadeca-1(16),3,6, 13(17),14-pentaene (E118) Compound was prepared using procedures similar to those used for preparing E5, starting from D145 and 2,4-dichloro-5- (trifluoromethyl) pyrimidine. LC-MS: 435.5 [M + H]⁺. ¹H NMR (400 MHz, CDCl₃): δ 10.72 (s, 1H), 8.02 (s, 1H), 7.00~6.82 (m, 1H), 6.53 (br, 1H), 4.44~4.36 (m, 3H), 4.12~4.08 (m, 2H), 3.67 (br, 2H), 3.55~3.49 (m, 2H), 2.31~2.23 (m, 2H), 1.90~1.83 (m, 4H), ¹⁹F NMR (400 MHz, CDCl₃): δ −62.03(s, 3F), −112.19 (s, 2F). |
| 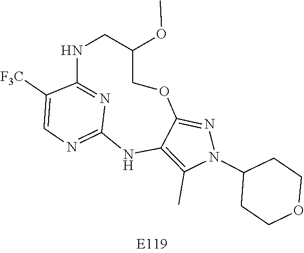 [SMILES: COC1CNc2nc(ncc2C(F)(F)F)Nc2c(nn(C3CCOCC3)c2C)OC1] | 10-Methoxy-4-methyl-5-(oxan-4-yl)-14-(trifluoromethyl)-8-oxa- 2,5,6,12,16,17-hexaazatricyclo[11.3.1.0^{3,7}]heptadeca-1(16),3,6, 13(17),14-pentaene (E119) Compound was prepared using procedures similar to those used for preparing E2, starting from D15 and 2,4-dichloro-5-(trifluoromethyl) pyrimidine. LC-MS: 429.5 [M + H]⁺. ¹H NMR (400 MHz, CDCl₃): δ 7.77 (s, 1H), 6.54~6.48 (m, 1H), 5.45 (s, 1H), 4.34 (br, 2H), 4.12~4.09(m, 3H), 3.50~3.44 (m, 4H), 2.30~2.20 (m, 5H), 1.79~1.75 (m, 6H). ¹⁹F NMR (400 MHz, CDCl₃): δ −61.03 (s, 3F). |
| 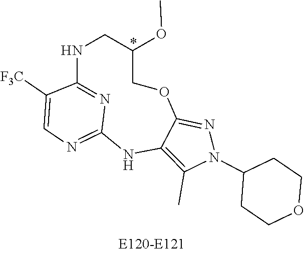 [SMILES: COC1CNc2nc(ncc2C(F)(F)F)Nc2c(nn(C3CCOCC3)c2C)OC1] | Enantiomers 1-2: 10-methoxy-4-methyl-5-(oxan-4-yl)-14-(trifluo romethyl)-8-oxa-2,5,6,12,16,17-hexaazatricyclo[11.3.1.0^{3,7}] heptadeca-1(16),3,6,13(17),14-pentaene (E120-E121) Compounds were prepared from chiral separation of E119. E120: LC-MS: 429.5 [M + H]⁺. ¹H NMR (400 MHz, CDCl₃): δ 8.01 (s, 1H), 5.80 (s, 1H), 4.47 (d, J = 12.0 Hz, 1H), 4.33 (t, J = 12.0 Hz, 1H), 4.30~4.07 (m, 3H), 4.01~3.96 (m, 1H), 3.53~3.50 (m, 2H), 3.42 (s, 3H), 3.35 (d, J = 4.0 Hz, 2H), 3.34~3.30 (m, 2H), 2.32~2.26 (m, 1H), 2.23 (s, 3H), 1.94~1.88 (m, 1H), 1.80~1.76 (m, 2H). ¹⁹F NMR (400 MHZ, CDCl₃): δ 61.03 (s, 3F). Chiral RT = 2.030 min (chiral method A); ee = 100%. E121: LC-MS: 429.5 [M + H]⁺. ¹H NMR (400 MHz, CDCl₃): δ 8.06 (s, 1H), 5.98 (s, 1H), 4.48 (d, J = 12.0 Hz, 1H), 4.32 (t, J = 12.0 Hz, 1H), 4.28~4.09 (m, 3H), 4.06~3.97 (m, 1H), 3.53~3.49 (m, 2H), 3.42 (s, 3H), 3.36 (d, J = 4.0 Hz, 2H), 3.35~3.31 (m, 2H), 2.32~2.26 (m, 1H), 2.16 (s, 3H), 1.94~1.88 (m, 1H), 1.79~1.75 (m, 2H). ¹⁹F NMR (400 |
| | MHz, CDCl₃): δ 61.03 (s, 3F). Chiral RT = 2.557 min (chiral method |
| | A); ee = 100%. |
| 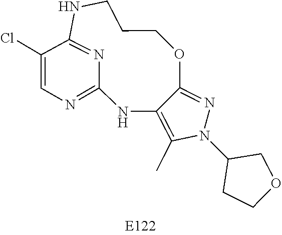 [SMILES: Cc1c2c(nn1C1CCOC1)OCCCNc1nc(ncc1Cl)N2] | 14-Chloro-4-methyl-5-(oxolan-3-yl)-8-oxa-2,5,6,12,16,17-hexa azatricyclo[11.3.1.0^{3,7}]heptadeca-1(16),3,6,13(17),14-pentaene (E122) Compound was prepared using procedures similar to those used for preparing E8, starting from tetrahydrofuran-3-ol. LCMS: 351.2 [M + H]⁺. ¹H NMR (400 MHz, CDCl₃): δ 7.9 (s, 1H), 6.32 (s, 1H), 5.51(s, 1H), 4.75~4.66 (m, 1H), 4.40~4.37 (m, 2H), 4.10~4.00 (m, 2H), 3.99~3.93 (m, 2H), 3.51~3.49 (m, 2H), 2.22~ 2.20 (m, 1H), 2.12 (m, 4H), 1.91~1.89 (m, 2H). |
| 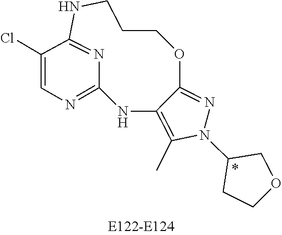 [SMILES: Cc1c2c(nn1C1CCOC1)OCCCNc1nc(ncc1Cl)N2] | Enantiomers 1-2: 14-chloro-4-methyl-5-(oxolan-3-yl)-8-oxa- 2,5,6,12,16,17-hexaazatricyclo[11.3.1.0^{3,7}]heptadeca- 1(16),3,6,13(17), 14-pentaene (E123-E124) Compounds were prepared from chiral separation of E122. E123: LCMS: 351.4 [M + H]⁺. ¹H NMR (400 MHz, CDCl₃): δ 7.79 (s, 1H), 6.59 (s, 1H), 5.64 (s, 1H), 4.74~4.70 (m, 1H), 4.39~4.37 (m, 3H), 4.17~4.07 (m, 2H), 3.99~3.91 (m, 2H), 3.54~3.50 (m, 2H), 2.43~2.40 (m, 1H), 2.31~2.22 (m, 4H), 1.93~1.90 (m, 2H). Chiral RT = 4.355 min (chiral method F); ee = 100%. E124: LCMS: 351.5 [M + H]⁺. ¹H NMR (400 MHz, CDCl₃): δ 7.79 (s, 1H), 6.57 (s, 1H), 5.62 (s, 1H), 4.74~4.70 (m, 1H), 4.39~4.37 (m, 3H), 4.17~4.11 (m, 2H), 3.99~3.91 (m, 2H), 3.54~3.50 (m, 2H), 2.43~2.40 (m, 1H), 2.31-2.22 (m, 4H), 1.93~1.90 (m, 2H). Chiral RT = 4.731 min (chiral method A); ee = 98.9%. |
| 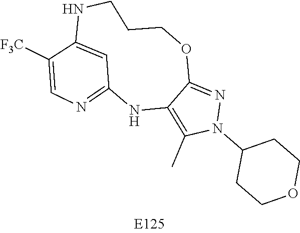 [SMILES: Cc1c2c(nn1C1CCOCC1)OCCCNc1cc(ncc1C(F)(F)F)N2] | 4-Methyl-5-(oxan-4-yl)-14-(trifluoromethyl)-8-oxa-2,5,6,12,16- pentaazatricyclo[11.3.1.0^{3,7}]heptadeca-1(16),3,6,13(17),14- pentaene (E125) Compound was prepared using procedures similar to those used for preparing E1, starting from 2,4-dichloro-5-(trifluoromethyl)pyridine and D7. LCMS: 398.5 [M + H]⁺. ¹H NMR (400 MHz, CD₃OD): δ 7.86 (s, 1H), 5.63 (s, 1H), 4.66~4.62 (m, 1H), 4.32~4.27 (m, 2H), 4.07~4.03 (m, 2H), 3.61~3.55 (m, 1H), 3.48~3.45 (m, 1H), 2.21~2.13 (m, 3H), 2.04~1.90(m, 2H), 1.81~1.79 (m, 2H). ¹⁹F NMR (400 MHz, CD₃OD): δ −62.4 (s, 3F). |
| 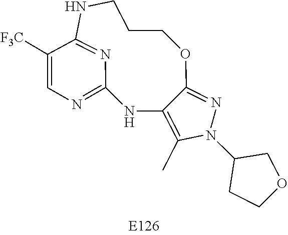 [SMILES: Cc1c2c(nn1C1CCOC1)OCCCNc1nc(ncc1C(F)(F)F)N2] | 4-Methyl-5-(oxolan-3-yl)-14-(trifluoromethyl)-8-oxa-2,5,6,12,16, 17-hexaazatricyclo[11.3.1.0^{3,7}]heptadeca-1(16),3,6,13(17),14- pentaene (E126) Compound was prepared using procedures similar to those used for preparing E1, starting from 2,4-dichloro-5-(trifluoromethyl)pyridine and tetrahydrofuran-3-ol. LCMS: 385.2 [M + H]⁺. ¹H NMR (400 MHz, CDCl₃): δ 8.06 (s, 1H), 6.82 (s, 1H), 5.58 (s, 1H), 4.75~4.71 (m, 1H), 4.40~4.38 (m, 2H), 4.18~4.06 (m, 2H), 3.99~3.92 (m, 2H), 3.54~3.50 (m, 2H), 2.43~ 2.37 (m, 2H), 2.30 (s, 3H), 2.06~1.91 (m, 2H). |
| 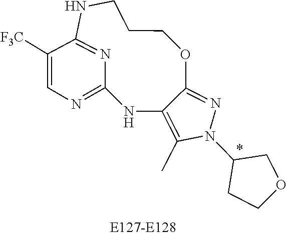 [SMILES: Cc1c2c(nn1C1CCOC1)OCCCNc1nc(ncc1C(F)(F)F)N2] | Enantiomers 1-2: 4-methyl-5-(oxolan-3-yl)-14-(trifluoromethyl)- 8-oxa-2,5,6,12,16,17-hexaazatricyclo[11.3.1.0^{3,7}]heptadeca- 1(16),3,6,13(17),14-pentaene (E127-E128) Compounds were prepared from chiral separation of E126. E127: LCMS: 385.3 [M + H]⁺. ¹H NMR (400 MHz, CDCl₃): δ 8.06 (s, 1H), 6.77 (s, 1H), 5.58 (s, 1H), 4.73~4.71 (m, 1H), 4.39~4.38 (m, 2H), 4.18~4.06 (m, 2H), 3.99~3.92 (m, 2H), 3.54~3.49 (m, 2H), 2.43~2.37 (m, 2H), 2.3(s, 3H), 1.96~1.82 (m, 2H). ¹⁹F NMR (400 MHz, CDCl₃): δ −60.9 (s, 3F). Chiral RT = 2.353 min (chiral method A); ee = 100%. E128: LCMS: 385.3 [M + H]⁺. ¹H NMR (400 MHz, CDCl₃): δ 8.06 (s, 1H), 6.82 (s, 1H), 5.58 (s, 1H), 4.75~4.71(m, 1H), 4.40~4.38(m, 2H), 4.18~4.06 (m, 2H), 3.99~3.92 (m, 2H), 3.54~3.50 (m, 2H), 2.43~2.37 (m, 2H), 2.3(s, 3H), 1.97~1.91 (m, 2H). ¹⁹F NMR (400 MHz, CDCl₃): δ −60.9 (s, 3F). Chiral RT = 3.581 min (chiral method A); ee = 100%. |
| 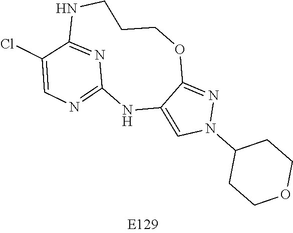 [SMILES: Clc1cnc2nc1NCCCOc1nn(C3CCOCC3)cc1N2] | 14-Chloro-5-(oxan-4-yl)-8-oxa-2,5,6,12,16,17-hexaazatricyclo [11.3.1.0^{3,7}]heptadeca-1(16),3,6,13(17),14-pentaene (E129) Compound was prepared using procedures similar to those used for preparing E9, starting from D36. LCMS: 351.2 [M + H]⁺. ¹H NMR (400 MHz, CDCl₃): δ 7.82 (s, 1H), 7.13 (s, 1H), 6.42 (br, 1H), 5.57 (br, 1H), 4.41~4.38 (m, 2H), 4.15~4.08 (m, 3H), 3.56~3.48 (m, 4H), 2.09~1.93 (m, 6H). |
| 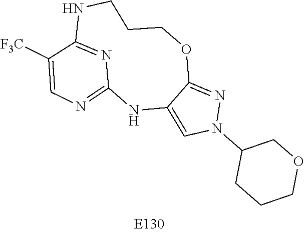 [SMILES: FC(F)(F)c1cnc2nc1NCCCOc1nn(C3CCCOC3)cc1N2] | 5-(Oxan-3-yl)-14-(trifluoromethyl)-8-oxa-2,5,6,12,16,17-hexaaza tricyclo[11.3.1.0^{3,7}]heptadeca-1(16),3,6,13(17),14-pentaene (E130) Compound was prepared using procedures similar to those used for preparing E9, starting from D24 and 2,4-dichloro-5- (trifluoromethyl)pyridine. LCMS: 385.2 [M + H]⁺. ¹H NMR (400 MHz, CDCl₃): δ 8.07 (s, 1H), 7.13 (s, 1H), 5.66 (br, 1H), 4.40~4.38 (m, 2H), 4.10~4.05 (m, 2H), 3.89~3.84 (m, 1H), 3.71~3.66 (m, 1H), 3.59~3.51 (m, 3H), 2.16~2.10 (m, 3H), 2.08~1.96 (m, 2H), 1.83~1.71 (m, 2H). ¹⁹F NMR (400 MHz, CDCl₃): δ −61.032 (s, 3F). |
| 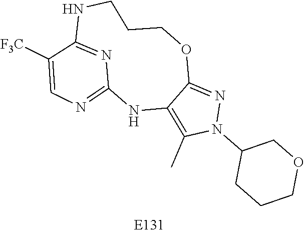 [SMILES: Cc1c2c(nn1C1CCCOC1)OCCCNc1nc(ncc1C(F)(F)F)N2] | 4-Methyl-5-(oxan-3-yl)-14-(trifluoromethyl)-8-oxa-2,5,6,12,16, 17-hexaazatricyclo[11.3.1.0^{3,7}]heptadeca-1(16),3,6,13(17),14- pentaene (E131) Compound was prepared using procedures similar to those used for preparing E8, starting from 2,4-dichloro-5-(trifluoromethyl)pyridine. LCMS: 399.2 [M + H]⁺. ¹H NMR (400 MHz, CDCl₃): δ 8.00 (s, 1H), 6.71 (s, 1H), 5.54 (br, 1H), 4.31~4.30 (m, 2H), 3.99~3.95 (m, 1H), 3.90~3.86 (m, 2H), 3.64~3.59 (m, 1H), 3.49~3.44 (m, 2H), 3.40~ 3.35 (m, 1H), 2.18~2.15 (m, 4H), 1.99~1.96 (m, 2H), 1.86~1.72 (m, 3H), ¹⁹F NMR (400 MHz, CDCl₃): δ −60.945 (s, 3F). |
| 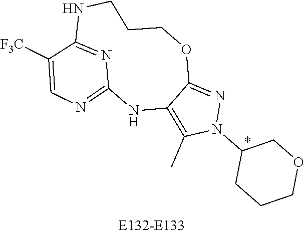 [SMILES: Cc1c2c(nn1C1CCCOC1)OCCCNc1nc(ncc1C(F)(F)F)N2] | Enantiomers 1-2: 4-methyl-5-(oxan-3-yl)-14-(trifluoromethyl)-8- oxa-2,5,6,12,16,17-hexaazatricyclo[11.3.1.0^{3,7}]heptadeca- 1(16),3,6,13(17),14-pentaene (E132-E133) Compounds were prepared from chiral separation of E131. E132: LC-MS: 399.3 [M + H]⁺. ¹H NMR (400 MHz, CDCl₃): δ 8.06 (s, 1H), 6.89 (s, 1H), 5.57 (br, 1H), 4.40~4.38 (m, 2H), 4.07~4.01 (m, 1H), 3.96~3.93 (m, 2H), 3.71~3.66 (m, 1H), 3.55~3.47 (m, 2H), 3.45~3.40 (m, 1H), 2.25~2.18 (m, 3H), 2.06~1.92 (m, 4H), 1.83~ 1.79 (m, 2H). ¹⁹F NMR (400 MHz, CDCl₃): δ −60.8.92 (s, 3F). Chiral RT = 2.030 min (chiral method A); ee = 100%. E133: LC-MS: 399.3 [M + H]⁺. ¹H NMR (400 MHz, CDCl₃): δ 8.06 (s, 1H), 6.87 (s, 1H), 5.58 (br, 1H), 4.39~4.38 (m, 2H), 4.09~4.01 (m, 1H), 3.97~3.93 (m, 2H), 3.71~3.66 (m, 1H), 3.55~3.51 (m, 2H), 3.47~3.40 (m, 1H), 2.30~2.03 (m, 3H), 2.02~1.94 (m, 4H), 1.93~ 1.81 (m, 2H). ¹⁹F NMR (400 MHz, CDCl₃): δ −60.887 (s, 3F). Chiral |
| | RT = 2.577 min (chiral method A); ee = 100%. |
| 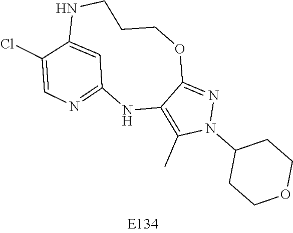 [SMILES: Cc1c2c(nn1C1CCOCC1)OCCCNc1cc(ncc1Cl)N2] | 14-Chloro-4-methyl-5-(oxan-4-yl)-8-oxa-2,5,6,12,16-pentaaza tricyclo[11.3.1.0^{3,7}]heptadeca-1(16),3,6,13(17),14-pentaene (E134) Compound was prepared using procedures similar to those used for preparing E8, starting from 2,5-dichloro-4-iodopyridine and D5. LC-MS: 364.5 [M + H]⁺. ¹H NMR (400 MHz, CDCl₃): δ 7.70 (s, 1H), 6.52 (s, 1H), 5.79 (s, 1H), 5.03 (m, 1H), 4.27~4.25 (m, 2H), 4.13~ 4.09 (m, 3H), 3.54~3.48 (m, 4H), 3.29~3.20 (m, 5H), 1.92 (m, 2H), 1.80~1.77 (m, 2H). |
| 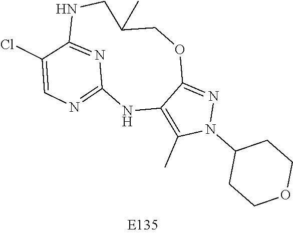 [SMILES: Cc1c2c(nn1C1CCOCC1)OCC(C)CNc1nc(ncc1Cl)N2] | 14-Chloro-4,10-dimethyl-5-(oxan-4-yl)-8-oxa-2,5,6,12,16,17- hexaazatricyclo[11.3.1.0^{3,7}]heptadeca-1(16),3,6,13(17),14- pentaene (E135) Compound was prepared using procedures similar to those used for preparing E5, starting from 3-amino-2-methylpropan-1-ol. LC-MS: 379.4 [M + H]⁺. ¹H NMR (400 MHz, CDCl₃): δ 7.77 (s, 1H), 6.54~6.48 (m, 1H), 5.45 (s, 1H), 4.34 (br, 2H), 4.12~4.09 (m, 3H), 3.50~3.44 (m, 4H), 2.30~2.20 (m, 5H), 1.79~1.75 (m, 6H). |
| 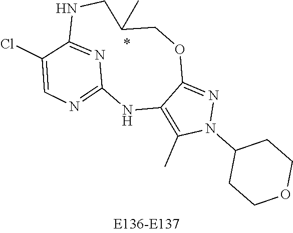 [SMILES: Cc1c2c(nn1C1CCOCC1)OCC(C)CNc1nc(ncc1Cl)N2] | Enantiomers 1-2: 14-chloro-4,10-dimethyl-5-(oxan-4-yl)-8-oxa- 2,5,6,12,16,17-hexaazatricyclo[11.3.1.0^{3,7}]heptadeca- 1(16),3,6,13(17),14-pentaene (E136-E137) Compounds were prepared from chiral separation of E135. E136: LC-MS: 379.2 [M + H]⁺. ¹H NMR (400 MHz, DMSO-d₆): δ 8.29 (s, 1H), 7.76 (s, 1H), 7.37 (t, J = 6.0 Hz, 1H), 4.20~3.92 (m, 5H), 3.55~3.40 (m, 3H), 2.90~2.86 (m, 1H), 2.15 (s, 3H), 2.00~1.91 (m, 2H), 1.73~1.62 (m, 3H), 0.91 (d, J = 7.6 Hz, 3H). Chiral RT = 4.822 min (chiral method A); ee = 100%. E137: LC-MS: 379.2 [M + H]⁺. ¹H NMR (400 MHz, DMSO-d₆): δ 8.29 (s, 1H), 7.76 (s, 1H), 7.38 (t, J = 6.0 Hz, 1H), 4.22~3.92 (m, 5H), 3.55~3.40 (m, 3H), 2.90~2.84 (m, 1H), 2.15 (s, 3H), 2.00~1.91 (m, 2H), 1.73~1.62 (m, 3H), 0.91 (d, J = 7.6 Hz, 3H). Chiral RT = 6.583 min (chiral method A); ee = 100%. |
| 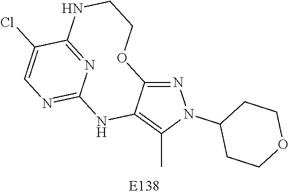 [SMILES: Cc1c2c(nn1C1CCOCC1)OCCNc1nc(ncc1Cl)N2] | 13-Chloro-4-methyl-5-(oxan-4-yl)-8-oxa-2,5,6,11,15,16-hexaaza tricyclo[10.3.1.0^{3,7}]hexadeca-1(15),3,6,12(16),13-pentaene (E138) Compound was prepared using procedures similar to those used for preparing E8, starting from 2-bromoethanamine hydrobromide and D5. LC-MS: 351.2 [M + H]⁺. ¹H NMR (400 MHz, CDCl₃): δ 7.77 (s, 1H), 6.20 (s, 1H), 5.45 (s, 1H), 4.47~4.46 (m, 2H), 4.12 (br, 1H), 4.09~ 4.06 (m, 2H), 3.67~3.66 (m, 2H), 3.54~3.48 (m, 2H), 2.30 (s, 3H), 2.24-2.17 (m, 2H), 1.80~1.77 (m, 2H). |
| 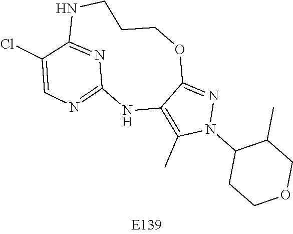 [SMILES: Cc1c2c(nn1C1CCOCC1C)OCCCNc1nc(ncc1Cl)N2] | 14-Chloro-4-methyl-5-(3-methyloxan-4-yl)-8-oxa-2,5,6,12,16,17- hexaazatricyclo[11.3.1.0^{3,7}]heptadeca-1(16),3,6,13(17),14- pentaene (E139) Compound was prepared using procedures similar to those used for preparing E8, starting from 3-methyltetrahydro-2H-pyran-4-ol. LC-MS: 379.2 [M + H]⁺. ¹H NMR (400 MHz, CDCl₃): δ 7.8 (s, 1H), 6.42 (s, 1H), 5.59 (s, 1H), 4.39~4.37 (m, 2H), 4.26~4.21 (m, 2H), 3.92~3.89 (m, 1H), 3.64~3.49 (m, 4H), 2.49~2.46 (m, 1H), 2.21 (s, 3H), 2.07~2.05 (m, 1H), 1.84~1.81 (m, 1H), 1.30~1.21 (m, 2H), 0.89~0.84 (m, 3H). |
| 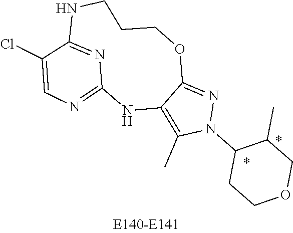 [SMILES: Cc1c2c(nn1C1CCOCC1C)OCCCNc1nc(ncc1Cl)N2] | Enantiomers 1-2: 14-chloro-4-methyl-5-(3-methyloxan-4-yl)-8- oxa-2,5,6,12,16,17-hexaazatricyclo[11.3.1.0^{3,7}]heptadeca- 1(16),3,6,13(17),14-pentaene (E140-E141) Compounds were prepared from chiral separation of E139. E140: LC-MS: 379.2 [M + H]⁺. ¹H NMR (400 MHz, CD₃OD): δ 7.70 (s, 1H), 4.46~4.41 (m, 1H), 4.35~4.33 (m, 2H), 4.20~4.17 (m, 1H), 3.86~3.83 (m, 1H), 3.69~3.58 (m, 2H), 3.42~3.39 (m, 2H), 2.49~ 2.44 (m, 1H), 2.23 (s, 3H), 2.09~2.07 (m, 1H), 1.88~1.76 (m, 3H), 0.84 (d, J = 8.0 Hz, 3H). Chiral RT = 4.006 min (chiral method A); ee = 98.7%. E141: LC-MS: 379.2 [M + H]⁺. ¹H NMR (400 MHz, CD₃OD): δ 7.71 (s, 1H), 4.46~4.41 (m, 1H), 4.35~4.32 (m, 2H), 4.21~4.16 (m, 1H), 3.86~3.82 (m, 1H), 3.69~3.58 (m, 2H), 3.42~3.40 (m, 2H), 2.48~ 2.44 (m, 1H), 2.23 (s, 3H), 2.09~2.07 (m, 1H), 1.88~1.76 (m, 3H), 0.84 (d, J = 8.0 Hz, 3H). Chiral RT = 4.569 min (chiral method A); |
| | ee = 100%. |
| 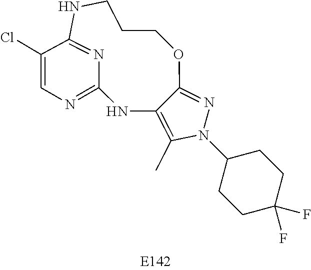 [SMILES: Cc1c2c(nn1C1CCC(F)(F)CC1)OCCCNc1nc(ncc1Cl)N2] | 14-Chloro-5-(4,4-difluorocyclohexyl)-4-methyl-8-oxa-2,5,6,12, 16,17-hexaazatricyclo[11.3.1.0^{3,7}]heptadeca-1(16),3,6,13(17),14- pentaene (E142) Compound was prepared using procedures similar to those used for preparing E1, starting from D146. LC-MS: 399.2 [M + H]⁺. ¹H NMR (400 MHz, CDCl₃): δ 7.92 (s, 1H), 6.95 (s, 1H), 5.72 (s, 1H), 4.43~4.33 (m, 2H), 4.02~3.95 (m, 1H), 3.61~3.55 (m, 2H), 2.39~2.31 (m, 7H), 1.99~1.85 (m, 6H). |
| 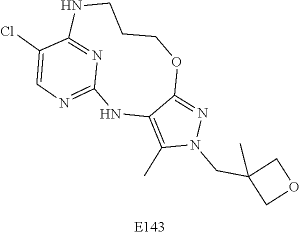 [SMILES: Cc1c2c(nn1CC1(C)COC1)OCCCNc1nc(ncc1Cl)N2] | 14-Chloro-4-methyl-5-[(3-methyloxetan-3-yl)methyl]-8-oxa-2,5, 6,12,16,17-hexaazatricyclo[11.3.1.0^{3,7}]heptadeca-1(16),3,6, 13(17), 14-pentaene (E143) Compound was prepared using procedures similar to those used for preparing E12, starting from D33 and (3-methyloxetan-3-yl)methyl methanesulfonate. LC-MS: 365.2 [M + H]⁺. ¹H NMR (400 MHz, CDCl₃): δ 7.81 (s, 1H), 6.17 (br, 1H), 5.55 (br, 1H), 4.77 (d, J = 6.0 Hz, 2H), 4.38 (d, J = 6.0 Hz, 2H), 4.34 (t, J = 4.4 Hz, 2H), 4.05 (s, 2H), 3.51 (dd, J = 10.8, 6.0 Hz, 2H), 2.18 (s, 3H), 1.91 (br, 2H), 1.26 (s, 3H). |
| 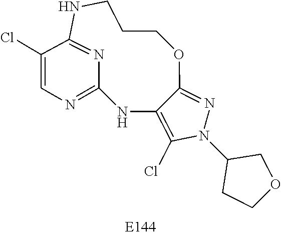 [SMILES: Clc1cnc2nc1NCCCOc1nn(C3CCOC3)c(Cl)c1N2] | 4,14-Dichloro-5-(oxolan-3-yl)-8-oxa-2,5,6,12,16,17-hexaaza tricyclo[11.3.1.0^{3,7}]heptadeca-1(16),3,6,13(17),14-pentaene (E144) Compound was prepared using procedures similar to those used for (preparing E1, starting from D155. LC-MS: 371.1[M + H]⁺. ¹H NMR (400 MHz, CDCl₃): δ 7.85 (s, 1H), 6.53 (br, 1H), 5.60 (br, 1H), 4.97~4.94 (m, 1H), 4.39~4.37 (m, 2H), 4.16~4.08 (m, 2H), 4.00~3.92 (m, 2H), 3.55~3.51 (m, 2H), 2.43~ 2.28 (m, 2H), 1.95~1.93 (m, 2H). |
| 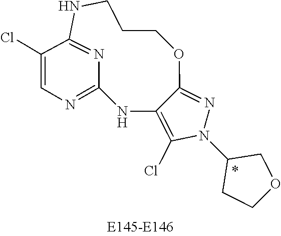 [SMILES: Clc1cnc2nc1NCCCOc1nn(C3CCOC3)c(Cl)c1N2] | Enantiomers 1-2: 4,14-Dichloro-5-(oxolan-3-yl)-8-oxa-2,5,6,12, 16,17-hexaaza tricyclo[11.3.1.0^{3,7}]heptadeca-1(16),3,6,13(17), 14-pentaene (E145-E146) Compounds were prepared from chiral separation of E144. E145: LC-MS: 371.1 [M + H]⁺. ¹H NMR (400 MHz, CDCl₃): δ 7.85 (s, 1H), 6.34 (br, 1H), 5.59~5.56 (m, 1H), 4.97~4.94 (m, 1H), 4.38 (t, J = 4.8 Hz, 2H), 4.15~4.08 (m, 2H), 4.00~3.94 (m, 2H), 3.56~3.51 (m, 2H), 2.43~2.28 (m, 2H), 1.94 (d, J = 4.0 Hz, 2H). Chiral RT = 3.619 min (chiral method A); ee = 100%. E146: LC-MS: 371.1 [M + H]⁺. ¹H NMR (400 MHz, CDCl₃): δ 7.85 (s, 1H), 6.51 (br, 1H), 5.57 (br, 1H), 4.97~4.92 (m, 1H), 4.37 (t, J = 4.4 Hz, 2H), 4.16~4.08 (m, 2H), 4.00~3.92 (m, 2H), 3.55~3.50 (m, 2H), 2.45~2.28 (m, 2H), 1.94 (d, J = 4.0 Hz, 2H). Chiral RT = 4.426 min (chiral method A); ee = 98.5%. |
| 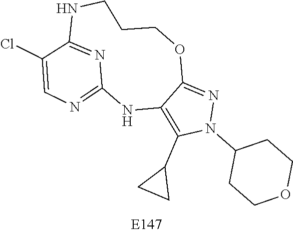 [SMILES: Clc1cnc2nc1NCCCOc1nn(C3CCOCC3)c(C3CC3)c1N2] | 14-Chloro-4-cyclopropyl-5-(oxan-4-yl)-8-oxa-2,5,6,12,16,17- hexaazatricyclo[11.3.1.0^{3,7}]heptadeca-1(16),3,6,13(17),14- pentaene (E147) Compound was prepared using procedures similar to those used for preparing E144, starting from D157. LC-MS: 391.2 [M + H]⁺. ¹H NMR (400 MHz, CDCl₃): δ 7.80 (s, 1H), 6.55 (br, 1H), 5.59 (br, 1H), 4.46 (br, 1H), 4.35 (br, 2H), 4.13~4.10 (m, 4H), 3.55~3.50 (m, 4H), 2.30~2.27 (m, 2H), 1.93 (br, 2H), 1.78~1.70 (m, 3H), 1.04~1.02 (m, 2H), 0.74~0.73 (m, 2H). |
| 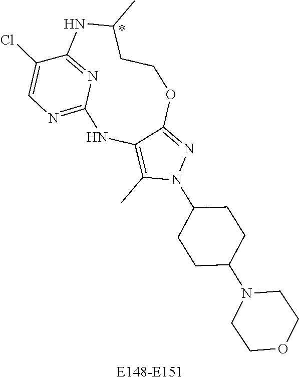 [SMILES: Cc1c2c(nn1C1CCC(N3CCOCC3)CC1)OCCC(C)Nc1nc(ncc1Cl)N2] | Enantiomers 1-4: 14-Chloro-4,11dimethyl-5-[4-(morpholin-4- ylcyclohexyl]-8-oxa-2,5,6,12,16,17-hexaazatricyclo[11.3.1.0^{3,7}] heptadeca1 (16),3,6,13(17),14-pentaene (E148-E151) Compounds were prepared using procedures similar to those used for preparing E55, starting from D94 and morpholine. E148: LC-MS: 462.2 [M + H]⁺. ¹H NMR (400 MHz, CDCl₃): δ 7.81 (s, 1H), 6.11 (br, 1H), 5.12 (d, J = 6.4 Hz, 1H), 4.48~4.31 (m, 2H), 4.08~3.95 (m, 1H), 3.87~3.65 (m, 5H), 2.61 (br, 4H), 2.39 (br, 1H), 2.18 (s, 3H), 2.13~1.76 (m, 8H), 1.42 (br, 2H), 1.31 (d, J = 6.8 Hz, 3H). Chiral RT = 2.792 min (chiral method B); ee = 100%. E149: LC-MS: 462.2 [M + H]⁺. ¹H NMR (400 MHz, CDCl₃): δ 7.82 (s, 1H), 6.12 (br, 1H), 5.12 (d, J = 6.4 Hz, 1H), 4.50~4.28 (m, 2H), 4.01 (dd, J = 3.2, 6.4 Hz, 2H), 3.79 (br, 4H), 2.54 (br, 4H), 2.35~ 2.09 (m, 8H), 1.94~1.79 (m, 2H), 1.72~1.42 (m, 4H), 1.32 (d, J = 6.8 Hz, 3H). Chiral RT = 2.808 min (chiral method B), ee = 100%. E150: LC-MS: 462.3 [M + H]⁺. ¹H NMR (400 MHz, CDCl₃): δ 7.82 (s, 1H), 6.08 (s, 1H), 5.12 (d, J = 6.4 Hz, 1H), 4.57~4.27 (m, 2H), 4.08~ 3.91 (m, 2H), 3.78 (t, J = 4.4 Hz, 4H), 2.51 (br, 4H), 2.36~2.07 (m, 8H), 1.92~1.81 (m, 2H), 1.70~1.41 (m, 4H), 1.32 (d, J = 6.8 Hz, 3H). Chiral RT = 3.737 min (chiral method B), 4.479(chiral method F); ee = 100%. E151: LC-MS: 462.2 [M + H]⁺. ¹H NMR (400 MHz, CDCl₃): δ 7.82 (s, 1H), 6.02 (s, 1H), 5.11 (d, J = 6.4 Hz, 1H), 4.53~4.24 (m, 2H), 4.02 (br, 1H), 3.88~3.64 (m, 5H), 2.58 (br, 4H), 2.43~2.29 (m, 1H), 2.18 (s, 3H), 2.12~1.72 (m, 8H), 1.46~1.35 (m, J = 8.4 Hz, 2H), 1.31 (d, J = 6.8 Hz, 3H). Chiral RT = 3.737 min (chiral method B), 5.175 |
| | (chiral method F); ee = 100%. |
| 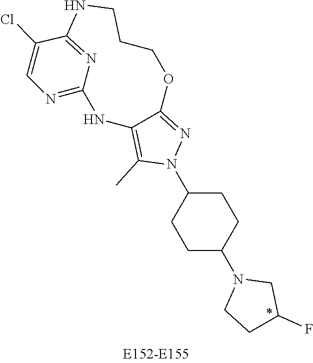 [SMILES: Cc1c2c(nn1C1CCC(N3CCC(F)C3)CC1)OCCCNc1nc(ncc1Cl)N2] | Enantiomers 1-4: 14-Chloro-5-[4-(3-fluoropyrrolidin-1-yl)cyclo hexyl]-4-methyl-8-oxa-2,5,6,12,16,17-hexaazatricyclo[11.3.1. 0^{3,7}]heptadeca-1(16),3,6,13(17),14-pentaene (E152-E155) Compounds were prepared using procedures similar to those used for preparing E48, starting from D85 and 3-fluoropyrrolidine. E152: LC-MS: 450.3 [M + H]⁺. ¹H NMR (400 MHz, CDCl₃): δ 7.81 (s, 1H), 6.07 (s, 1H), 5.53 (t, J = 6.4 Hz, 1H), 5.19 (dt, J = 56.4 Hz, 4.4 Hz, 1H), 4.38 (t, J = 4.6 Hz, 2H), 3.89~3.81 (m, 1H), 3.54~3.49 (m, 2H), 3.05~2.91 (m, 2H), 2.85~2.74 (m, 1H), 2.55~2.48 (m, 1H), 2.19 (s, 3H), 2.13~1.99 (m, 6H), 1.96~1.89 (m, 5H), 1.44~1.38 (m, 2H). ¹⁹F NMR (376 MHz, CDCl₃): δ −167.23. Chiral RT = 4.88 7 min (chiral method F), 5.466 min (chiral method A); ee = 100%. E153: LC-MS: 450.3 [M + H]⁺. ¹H NMR (400 MHz, CDCl₃): δ 7.81 (s, 1H), 6.07 (s, 1H), 5.53 (t, J = 6.4 Hz, 1H), 5.25~5.12 (m, 1H), 4.38 (t, J = 4.4 Hz, 2H), 3.90~3.79 (m, 1H), 3.50 (q, J = 9.6 Hz, 2H), 3.06~2.86 (m, 2H), 2.84~2.70 (m, 1H), 2.46 (br, 1H), 2.19 (s, 3H), 2.14~1.96 (m, 6H), 1.91~1.90 (m, 5H), 1.46~1.42 (m, 2H). ¹⁹F NMR (376 MHz, CDCl₃): δ −167.23. Chiral RT = 4.887 min (chiral method F), 7.536 min (chiral method A); ee = 100%. E154: LC-MS: 450.3 [M + H]⁺. ¹H NMR (400 MHz, CDCl₃): δ 7.80 (s, 1H), 6.06 (s, 1H), 5.52 (t, J = 6.4 Hz, 1H), 5.26-5.14 (m, 1H), 4.39 (t, J = 4.6 Hz, 2H), 3.96~3.87 (m, 1H), 3.51 (q, J = 5.6 Hz, 2H), 3.03~ 2.88 (m, 1H), 2.82~2.70 (m, 2H), 2.62~2.57 (m, 1H), 2.33~2.23 (m, |
| | 3H), 2.19 (s, 3H), 2.16~1.99 (m, 4H), 1.93~1.90 (m, 2H), 1.64~1.61 |
| | (m, 2H), 1.56~1.49 (m, 2H). ¹⁹F NMR (376 MHz, CDCl₃): δ −167.46. |
| | Chiral RT = 5.305 min (chiral method F); ee = 100%. |
| | E155: LC-MS: 450.3 [M + H]⁺. ¹H NMR (400 MHz, CDCl₃): δ 7.81 (s, |
| | 1H), 6.09 (s, 1H), 5.54 (t, J = 9.6 Hz, 1H), 5.27-5.13 (m, 1H), 4.40 (t, |
| | J = 4.4 Hz, 2H), 3.98~3.88 (m, 1H), 3.53 (q, J = 6.0 Hz, 2H), 3.03~ |
| | 2.89 (m, 1H), 2.86~2.70 (m, 2H), 2.63~2.58 (m, 1H), 2.34-2.21 (m, |
| | 3H), 2.20 (s, 3H), 2.18~2.00 (m, 4H), 1.93~1.90 (m, 2H), 1.64~1.61 |
| | (m, 2H), 1.57~1.50 (m, 2H). ¹⁹F NMR (376 MHz, CDCl₃): δ −167.45. |
| | Chiral RT = 5.461 min (chiral method F); ee = 100%. |
| 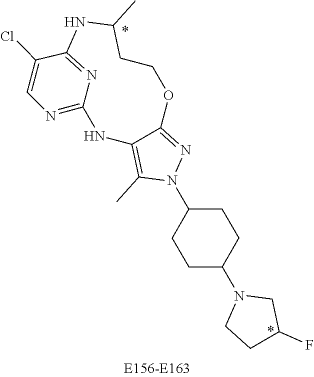 [SMILES: Cc1c2c(nn1C1CCC(N3CCC(F)C3)CC1)OCCC(C)Nc1nc(ncc1Cl)N2] | Enantiomers 1-8: 14-Chloro-5-[4-(3-fluoropyrrolidin-1-yl) cyclo hexyl]-4,11-dimethyl-8-oxa-2,5,6,12,16,17-hexaazatricyclo [11.3.1.0^{3,7}]heptadeca-1(16),3,6,13(17),14-pentaene (E156-E163) Compounds were prepared using procedures similar to those used for preparing E55, starting from D94 and 3-fluoropyrrolidine. E156: LC-MS: 464.3 [M + H]⁺. ¹H NMR (400 MHz, CDCl₃): δ 7.82 (s, 1H), 6.05 (s, 1H), 5.30-5.05 (m, 2H), 4.54~4.23 (m, 2H), 4.07~3.74 (m, 2H), 3.10~2.67 (m, 3H), 2.57~2.43 (m, 1H), 2.18 (s, 3H), 2.14~ 1.75 (m, 11H), 1.48~1.36 (m, 2H), 1.31 (d, J = 6.8 Hz, 3H). Chiral RT = 2.744 min (chiral method A); ee = 100%. E157: LC-MS: 464.2 [M + H]⁺. ¹H NMR (400 MHz, CDCl₃): δ 7.82 (s, 1H), 6.07 (br, 1H), 5.32~5.02 (m, 2H), 4.54~4.24 (m, 2H), 4.08~ 3.79 (m, 2H), 3.06~2.88 (m, 2H), 2.87~2.69 (m, 1H), 2.52 (d, J = 6.0 Hz, 1H), 2.26~1.77 (m, 14H), 1.49~1.36 (m, 2H), 1.31 (d, J = 6.8 Hz, 3H). Chiral RT = 3.849 min (chiral method A); ee = 100%. E158: LC-MS: 464.3 [M + H]⁺. ¹H NMR (400 MHz, CDCl₃): δ 7.81 (s, 1H), 6.04 (s, 1H), 5.32~5.06 (m, 2H), 4.54~4.27 (m, 2H), 4.08~3.83 (m, 2H), 3.00~2.55 (m, 4H), 2.45-2.22 (m, 3H), 2.19 (s, 3H), 2.15~ 1.98 (m, 3H), 1.93~1.78 (m, 2H), 1.64~1.48 (m, 4H), 1.31 (d, J = 6.4Hz, 3H). Chiral RT = 6.065 min (chiral method A); ee = 100%. E159: LC-MS: 464.2 [M + H]⁺. ¹H NMR (400 MHz, CDCl₃): δ 7.81 (s, 1H), 6.06 (br, 1H), 5.32~5.01 (m, 2H), 4.57~4.27 (m, 2H), 4.11~ 3.86 (m, 2H), 3.07~2.53 (m, 4H), 2.43~2.24 (m, 3H), 2.20 (s, 3H), 2.11~1.98 (m, 3H), 1.94~1.85 (m, 2H), 1.70~1.50 (m, 4H), 1.32 (d, |
| | J = 6.0 Hz, 3H). Chiral RT = 6.985 min (chiral method A), 2.568 min |
| | (chiral method A); ee = 100%. |
| | E160: LC-MS: 464.2 [M + H]⁺. ¹H NMR (400 MHz, CDCl₃): δ 7.81 (s, |
| | 1H), 6.07 (s, 1H), 5.35~5.04 (m, 2H), 4.53~4.27 (m, 2H), 4.07~3.85 |
| | (m, 2H), 3.07~2.56 (m, 4H), 2.43~2.23 (m, 3H), 2.19 (s, 3H), 2.14~ |
| | 1.98 (m, 3H), 1.94~1.85 (m, 2H), 1.70~1.50 (m, 4H), 1.31 (d, J = 7.8 |
| | Hz, 3H). Chiral RT = 6.985 min (chiral method A), 3.107 min (chiral |
| | method A); ee = 100%. |
| | E161: LC-MS: 464.3 [M + H]⁺. ¹H NMR (400 MHz, CDCl₃): δ 7.81 (s, |
| | 1H), 6.05 (s, 1H), 5.32~5.06 (m, 2H), 4.56~4.27 (m, 2H), 4.07~3.85 |
| | (m, 2H), 3.02~2.52 (m, 4H), 2.43~2.21 (m, 3H), 2.19 (s, 3H), |
| | 2.15~1.96 (m, 3H), 1.93~1.84 (m, 2H), 1.70~1.47 (m, 4H), 1.31 (d, |
| | J = 6.8 Hz, 3H). Chiral RT = 8.389 min (chiral method A); ee = 100%. |
| | E162: LC-MS: 464.2 [M + H]⁺. ¹H NMR (400 MHz, CDCl₃): δ 7.81 (s, |
| | 1H), 6.07 (s, 1H), 5.31~5.04 (m, 2H), 4.54~4.23 (m, 2H), 4.08~3.76 |
| | (m, 2H), 3.11~2.69 (m, 3H), 2.59~2.38 (m, J = 6.4 Hz, 1H), 2.18 (s, |
| | 3H), 2.15~1.72 (m, 11H), 1.48~1.37 (m, 2H), 1.31 (d, J = 6.8 Hz, |
| | 3H). Chiral RT = 8.759 min (chiral method A); ee = 100%. |
| | E163: LC-MS: 464.3 [M + H]⁺. ¹H NMR (400 MHz, CDCl₃): δ 7.81 (s, |
| | 1H), 6.11 (s, 1H), 5.25~5.11 (m, 2H), 4.54~4.21 (m, 2H), 4.10~3.67 |
| | (m, 2H), 3.11~2.69 (m, 3H), 2.57~2.41 (m, 1H), 2.24~1.79 (m, 14H), |
| | 1.48~1.36 (m, 2H), 1.31 (d, J = 7.2 Hz, 3H). Chiral RT = 9.940 min |
| | (chiral method A); ee = 100%. |
| 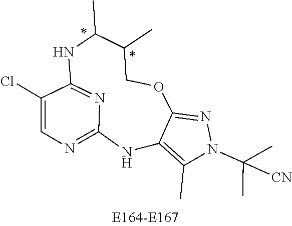 [SMILES: Cc1c2c(nn1C(C)(C)C#N)OCC(C)C(C)Nc1nc(ncc1Cl)N2] | Enantiomers 1-4: 2-{14-Chloro-4,10,11-trimethyl-8-oxa-2,5,6, 12,16,17-hexaazatricyclo[11.3.1.0^{3,7}]heptadeca-1(16),3,6, 13(17),14-pentaen-5-yl}-2-methylpropanenitrile (E164-E167) Compounds were prepared using procedures similar to those used for preparing E43. E164: LC-MS: 376.3 [M + H]⁺. ¹H NMR (400 MHz, CDCl₃): δ 7.82 (s, 1H), 6.29 (s, 1H), 4.87 (d, J = 6.8 Hz, 1H), 4.36~4.11 (m, 3H), 2.46 (s, 3H), 1.96 (s, 3H), 1.92 (s, 3H), 1.90~1.84 (m, 1H), 1.32 (d, J = 7.2 Hz, 3H), 0.82 (d, J = 6.8 Hz, 3H). Chiral RT = 2.942 min (chiral method A); ee = 100%. E165: LC-MS: 464.2 [M + H]⁺. ¹H NMR (400 MHz, CDCl₃): δ 7.84 (s, 1H), 6.13 (s, 1H), 5.18 (d, J = 7.6 Hz, 1H), 4.35~4.18 (m, 2H), 4.14~ 4.01 (m, 1H), 2.46 (s, 3H), 2.05~1.99 (m, 1H), 1.98 (s, 3H), 1.91 (s, 3H), 1.19 (d, J = 7.6 Hz, 3H), 0.93 (d, J = 7.6 Hz, 3H). Chiral RT = 4.144 min (chiral method A); ee = 99.5%. |
| | E126: LC-MS: 376.3[M + H]⁺. ¹H NMR (400 MHz, CDCl₃): δ 7.82 (s, |
| | 1H), 6.18 (s, 1H), 4.87 (d, J = 6.8 Hz, 1H), 4.32~4.19 (m, 3H), 2.46 |
| | (s, 3H), 1.96 (s, 3H), 1.92 (s, 3H), 1.90~1.84 (m, 1H), 1.32 (d, J = |
| | 6.8 Hz, 3H), 0.82 (d, J = 6.8 Hz, 3H). Chiral RT = 4.732 min (chiral |
| | method A); ee = 100%. |
| | E167: LC-MS: 376.3[M + H]⁺. ¹H NMR (400 MHz, CDCl₃): δ 7.84 (s, |
| | 1H), 6.15 (s, 1H), 5.18 (d, J = 7.6 Hz, 1H), 4.34~4.20 (m, 2H), 4.14 ~ |
| | 4.04 (m, 1H), 2.46 (s, 3H), 2.06~1.99 (m, 1H), 1.98 (s, 3H), 1.91 |
| | (s, 3H), 1.19 (d, J = 7.6 Hz, 3H), 0.93 (d, J = 7.6 Hz, 3H). Chiral RT = |
| | 5.656 min (chiral method A); ee = 100%. |
| 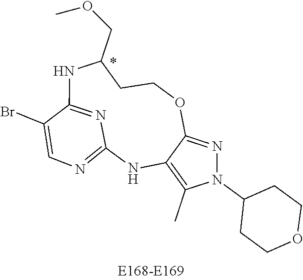 [SMILES: COCC1CCOc2nn(C3CCOCC3)c(C)c2Nc2ncc(Br)c(n2)N1] | Enantiomers 1-2: 14-Bromo-11-(methoxymethyl)-4-methyl-5- (oxan-4-yl)-8-oxa-2,5,6,12,16,17-hexaazatricyclo[11.3.1.0^{3,7}] heptadeca-1(16),3,6,13(17),14-pentaene (E168-E169) Compounds were prepared using procedures similar to those used for preparing E35, starting from 5-bromo-2,4-dichloropyrimidine. E168: LC-MS: 453.2 [M + H]⁺. ¹H NMR (400 MHz, CDCl₃): δ 7.91 (s, 1H), 6.18 (s, 1H), 5.69 (d, J = 7.2 Hz, 1H), 4.50~4.46 (m, 1H), 4.36~ 4.31 (m, 1H), 4.12~4.05 (m, 4H), 3.54~3.45 (m, 4H), 3.41 (s, 3H), 2.31~2.20 (m, 5H), 2.05-2.01 (m, 1H), 1.82~1.73 (m, 3H). Chiral RT = 2.382 min (chiral method B); ee = 99.7%. E169: LC-MS: 453.2 [M + H]⁺. ¹H NMR (400 MHz, CDCl₃): δ 7.86 (s, 1H), 5.97 (d, J = 6.8 Hz, 1H), 4.48 (dd, J = 11.6, 2.8 Hz, 1H), 4.34~ 4.28 (m, 1H), 4.12~4.05 (m, 4H), 3.54~3.46 (m, 4H), 3.42 (s, 3H), 2.31~2.21 (m, 5H), 2.07~2.03(m, 1H), 1.81~1.74 (m, 3H). Chiral RT = 3.057 min (chiral method B); ee = 99.8%. |
| 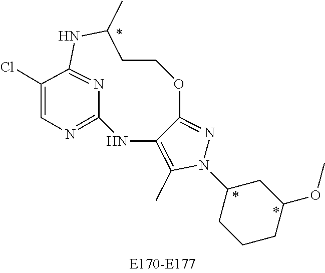 [SMILES: COC1CCCC(n2nc3c(c2C)Nc2ncc(Cl)c(n2)NC(C)CCO3)C1] | Enantiomers 1-8: 14-Chloro-5-(3-methoxycyclohexyl)-4,11- dimethyl-8-oxa-2,5,6,12,16,17-hexaazatricyclo[11.3.1.0^{3,7}] heptadeca-1(16),3,6,13(17),14-pentaene (E170-E177) Compounds were prepared using procedures similar to those used for preparing E79, starting from D73 and 3-methoxycyclohexyl methanesulfonate. E170: LC-MS: 407.3 [M + H]⁺. ¹H NMR (400 MHz, CD₃OD): δ 7.72 (s, 1H), 4.41 (d, J = 6.0 Hz, 2.4 Hz, 1H), 4.27 (t, J = 9.6 Hz, 2H), 3.95~ 3.93 (m, 1H), 3.74 (br, 1H), 3.35 (s, 3H), 2.19 (s, 3H), 2.09~1.95 (m, 3H), 1.82~1.72 (m, 5H), 1.66~1.41 (m, 2H), 1.29 (d, J = 6.8 Hz, 3H). Chiral RT = 2.920 min (chiral method F); ee = 100%. E171: LC-MS: 407.3 [M + H]⁺. ¹H NMR (400 MHz, CD₃OD): δ 7.72 (s, 1H), 4.42~4.38 (m, 1H), 4.27 (td, J = 8.4 Hz, 2.0 Hz, 2H), 3.95~3.93 (m, 1H), 3.74 (br, 1H), 3.35 (s, 3H), 2.19 (s, 3H), 2.09~1.95 (m, 3H), 1.82~1.72 (m, 5H), 1.66~1.41 (m, 2H), 1.29 (d, J = 6.8 Hz, 3H). Chiral RT = 3.071 min (chiral method F); ee = 100%. E172: LC-MS: 407.3 [M + H]⁺. ¹H NMR (400 MHz, CD₃OD): δ 7.64 |
| | (s, 1H), 4.32 (d, J = 12.0 Hz, 1H), 4.19 (t, J = 9.6 Hz, 2H), 3.85~3.80 |
| | (m, 1H), 3.64 (br, 1H), 3.30 (s, 3H), 2.10 (s, 3H), 1.93~1.78 (m, 5H), |
| | 1.71~1.57 (m, 4H), 1.34~1.32 (m, 1H), 1.21 (d, J = 6.8 MHz, 3H). |
| | Chiral RT = 3.10 min (chiral method F), 4.10 min (chiral method A); |
| | ee = 100%. |
| | E173: LC-MS: 407.3 [M + H]⁺. ¹H NMR (400 MHz, CD₃OD): δ 7.72 |
| | (s, 1H), 4.42~4.39 (m, 1H), 4.29~4.24 (m, 2H), 3.95~3.91 (m, 1H), |
| | 3.73 (br, 1H), 3.35 (s, 3H), 2.19 (s, 3H), 2.02~1.65 (m, 10H), |
| | 1.45~1.37 (m, 1H), 1.29 (d, J = 6.8 Hz, 3H). Chiral RT = 3.135 min |
| | (chiral method F); ee = 100%. |
| | E174: LC-MS: 407.3 [M + H]⁺. ¹H NMR (400 MHz, CD₃OD): δ 7.72 |
| | (s, 1H), 4.41~4.38 (m, 1H), 4.29 (t, J = 12.0 Hz, 1H), 4.08~4.03 (m, |
| | 1H), 3.95~3.92 (m, 1H), 3.35 (s, 3H), 3.33~3.30 (m, 1H), 2.28 (d, J = |
| | 12.0 Hz, 1H), 2.20 (s, 3H), 2.10 (d, J = 12.0 Hz, 1H), 1.90~1.72 (m, |
| | 6H), 1.43~1.40 (m, 1H), 1.30 (d, J = 6.8 Hz, 3H), 1.21~1.14 (m, 1H). |
| | Chiral RT = 3.272 min (chiral method F); ee = 100%. |
| | E175: LC-MS: 407.3 [M + H]⁺. ¹H NMR (400 MHz, CD₃OD): δ 7.62 |
| | (s, 1H), 4.30 (dq, J = 12.0, 2.0 Hz, 1H), 4.18 (td, J = 8.8, 2.4 Hz, |
| | 1H), 4.00~3.92 (m, 1H), 3.85~3.82(m, 1H), 3.27 (s, 3H), 3.24~3.21 |
| | (m, 1H), 2.10 (s, 3H), 2.12~2.08 (m, 1H), 2.01 (d, J = 7.6 Hz, 1H), |
| | 1.83~1.61 (m, 6H), 1.34~1.31 (m, 1H), 1.19 (d, J = 7.2 Hz, 3H), |
| | 1.08~1.05(m, 1H). Chiral RT = 3.484 min (chiral method F); ee = |
| | 100%. |
| | E176: LC-MS: 407.3 [M + H]⁺. ¹H NMR (400 MHz, CD₃OD): δ 7.72 |
| | (s, 1H), 4.41~4.38 (m, 1H), 4.28 (t, J = 9.6 Hz, 1H), 4.06~4.04 (m, |
| | 1H), 3.98~3.90 (m, 1H), 3.38 (s, 3H), 3.33~3.30 (m, 1H), 2.27 (d, J = |
| | 12.0 Hz, 1H), 2.20 (s, 3H), 2.12 (d, J = 12.0 H, 1H), 1.91~1.69 (m, |
| | 6H), 1.43~1.40 (m, 1H), 1.30 (d, J = 7.2 Hz, 3H), 1.18~1.14 (m, 1H). |
| | Chiral RT = 3.571 min (chiral method F); ee = 99.1%. |
| | E177: LC-MS: 407.4 [M + H]⁺. ¹H NMR (400 MHz, CD₃OD): δ 7.72 |
| | (s, 1H), 4.42~4.38 (m, 1H), 4.30~4.25 (m, 1H), 4.07~4.02 (m, 1H), |
| | 3.95~3.91 (m, 1H), 3.37 (s, 3H), 3.33~3.30 (m, 1H), 2.20 (s, 3H), |
| | 2.20~2.14 (m, 1H), 2.12~2.10 (m, 1H), 1.93~1.69 (m, 6H), 1.44~ |
| | 1.41 (m, 1H), 1.29 (d, J = 6.8 Hz, 3H), 1.18~1.14 (m, 1H). Chiral |
| | RT = 3.830 min (chiral method F); ee = 100% |
| 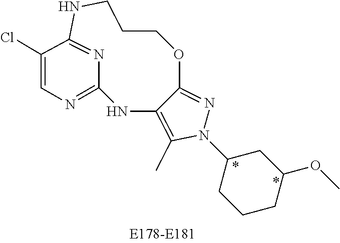 [SMILES: COC1CCCC(n2nc3c(c2C)Nc2ncc(Cl)c(n2)NCCCO3)C1] | Enantiomers 1-4: 14-Chloro-5-(3-methoxycyclohexyl)-4-methyl- 8-oxa-2,5,6,12,16,17-hexaazatricyclo[11.3.1.0^{3,7}]heptadeca- 1(16),3,6,13(17),14-pentaene (E178-E181) Compounds were prepared using procedures similar to those used for preparing E12, starting from D33 and 3-methoxycyclohexyl methanesulfonate. E178: LC-MS: 393.3 [M + H]⁺. ¹H NMR (400 MHz, CDCl₃): δ 7.81 (s, 1H), 6.12 (s, 1H), 5.53 (t, J = 5.6 Hz, 1H), 4.38 (t, J = 5.2 Hz, 2H), 4.25~4.18(m, 1H), 3.71 (br, 1H), 3.54~3.45 (m, 2H), 3.33 (s, 3H), 2.19 (s, 3H), 2.12~1.99 (m, 2H), 1.94~1.84 (m, 6H), 1.75~1.61 (m, 1H), 1.44~1.38 (m, 1H). Chiral RT = 2.363 min (chiral method G); ee = 100%. E179: LC-MS: 393.3 [M + H]⁺. ¹H NMR (400 MHz, CDCl₃): δ 7.80 (s, 1H), 6.35 (s, 1H), 5.55 (s, 1H), 4.39 (d, J = 4.0 Hz, 2H), 4.25~4.19 (m, 1H), 3.71 (br, 1H), 3.52 (d, J = 4.8 Hz, 2H), 3.33 (s, 3H), 2.19 (s, 3H), 2.12~2.00 (m, 2H), 1.91~1.86 (m, 6H), 1.78~1.66 (m, 1H), |
| | 1.43~1.36 (m, 1H). Chiral RT = 3.676 min (chiral method G); ee = |
| | 100%. |
| | E180: LC-MS: 393.3 [M + H]⁺. ¹H NMR (400 MHz, CDCl₃): δ 7.78 (s, |
| | 1H), 6.76 (s, 1H), 5.69 (t, J = 6.4 Hz, 1H), 4.38 (t, J = 4.8 Hz, 2H), |
| | 3.89~3.85(m, 1H), 3.53 (d, J = 6.0 Hz, 2H), 3.37 (s, 3H), 3.28~3.20 |
| | (m, 1H), 2.29~2.25 (m, 1H), 2.21 (s, 3H), 2.11~2.07 (m, 1H), |
| | 1.98~1.80 (m, 6H), 1.36~1.20 (m, 2H). Chiral RT = 3.770 min (chiral |
| | method G); ee = 100%. |
| | E181: LC-MS: 393.3 [M + H]⁺. ¹H NMR (400 MHz, CDCl₃): δ 7.81 (s, |
| | 1H), 6.13 (s, 1H), 5.54 (s, 1H), 4.39 (t, J = 4.8 Hz, 2H), 3.89~3.85 |
| | (m, 1H), 3.52 (d, J = 4.0 Hz, 2H), 3.37 (s, 3H), 3.28~3.20 (m, 1H), |
| | 2.29~2.26 (m, 1H), 2.20 (s, 3H), 2.11~2.08 (m, 1H), 1.94~1.80 (m, |
| | 6H), 1.36~1.20 (m, 2H). Chiral RT = 7.785 min (chiral method G); |
| | ee = 100%. |
| 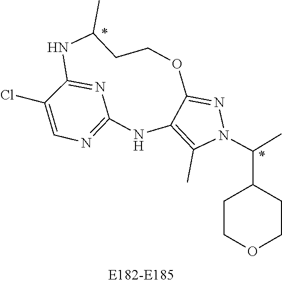 [SMILES: Cc1c2c(nn1C(C)C1CCOCC1)OCCC(C)Nc1nc(ncc1Cl)N2] | Enantiomers 1-4: 14-Chloro-4,11-dimethyl-5-[1-(oxan-4-yl)ethyl]- 8-oxa-2,5,6,12,16,17-hexaazatricyclo[11.3.1.0^{3,7}]heptadeca- 1(16),3,6,13(17),14-pentaene (E182-E185) Compounds were prepared using procedures similar to those used for preparing E78, starting from D73 and 1-(tetrahydro-2H-pyran-4- yl)ethyl methanesulfonate. E182: LC-MS: 407.3 [M + H]⁺. ¹H NMR (400 MHz, CDCl₃): δ 7.82 (s, 1H), 6.04 (s, 1H), 5.11 (s, 1H), 4.51~4.34 (m, 1H), 4.30~4.25 (m, 1H), 4.09~3.93 (m, 3H), 3.78~3.73 (m, 1H), 3.43~3.33 (m, 2H), 2.30 (s, 3H), 2.16~1.99 (m, 1H), 1.93~1.83 (m, 2H), 1.76~1.73 (m, 2H), 1.59~1.51 (m, 3H), 1.50 (d, J = 4.8 Hz, 3H), 1.47~1.39 (m, 2H). Chiral RT = 2.471 min (chiral method A); ee = 100%. E183: LC-MS: 407.2 [M + H]+. ¹H NMR (400 MHz, CDCl₃): δ 7.82 (s, 1H), 6.11 (s, 1H), 5.12 (d, J = 6.0 Hz, 1H), 4.50 (dd, J = 3.2, 3.6 Hz, 1H), 4.29 (t, J = 20.8 Hz, 1H), 4.09~4.01 (m, 2H), 3.98~3.93 (m, 1H), 3.78~3.73 (m, 1H), 3.40 (t, J = 22.0 Hz, 1H), 3.35~3.29 (m, 1H), 2.16 (s, 3H), 2.13 (br, 1H), 1.93~1.83 (m, 2H), 1.76~1.73 (m, 2H), 1.59~1.51 (m, 3H), 1.49 (d, J = 4.0 Hz, 3H), 1.46~1.38 (m, 2H). Chiral RT = 2.967 min (chiral method A); ee = 98.5%. |
| | E184: LC-MS: 407.3 [M + H]⁺. ¹H NMR (400 MHz, CDCl₃): δ 7.82 (s, |
| | 1H), 6.03 (s, 1H), 5.12 (d, J = 6.8 Hz, 1H), 4.49 (dd, J = 3.2, 2.8 Hz, |
| | 1H), 4.32 (t, J = 21.2 Hz, 1H), 4.02~3.99 (m, 2H), 3.89~3.86 (m, 1H), |
| | 3.74~3.70 (m, 1H), 3.41~3.27 (m, 2H), 2.16 (s, 3H), 2.06~2.01 (m, |
| | 1H), 1.93~1.83 (m, 2H), 1.76~1.73 (m, 2H), 1.59~1.51 (m, 3H), 1.50 |
| | (d, J = 4.0 Hz, 3H), 1.40~1.38 (m, 2H). Chiral RT = 4.335 min (chiral |
| | method A); ee = 100%. |
| | E185: LC-MS: 407.3 [M + H]⁺. ¹H NMR (400 MHz, CDCl₃): δ 7.82 (s, |
| | 1H), 6.04 (s, 1H), 5.12 (d, J = 6.8 Hz, 1H), 4.49 (dd, J = 6.8, 4.8 Hz, |
| | 1H), 4.41 (t, J = 15.2 Hz, 1H), 4.32~4.01 (m, 2H), 4.00~3.89 (m, 1H), |
| | 3.86~3.68 (m, 1H), 3.41~3.27 (m, 2H), 2.16 (s, 3H), 2.09~2.01 (m, |
| | 1H), 1.92~1.86 (m, 2H), 1.83~1.60 (m, 2H), 1.59~1.51 (m, 3H), 1.48 |
| | (d, J = 4.8 Hz, 3H), 1.40~1.38 (m, 2H). Chiral RT = 6.023 min (chiral |
| | method A); ee = 100%. |
| 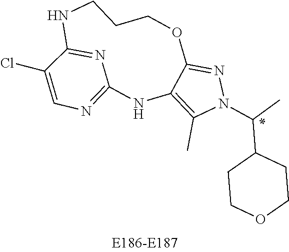 [SMILES: Cc1c2c(nn1C(C)C1CCOCC1)OCCCNc1nc(ncc1Cl)N2] | Enantiomers 1-2: 14-Chloro-4-methyl-5-[1 -(oxan-4-yl)ethyl]-8- oxa-2,5,6,12,16,17-hexaazatricyclo[11.3.1.0^{3,7}]heptadeca-1(16), 3,6,13(17),14-pentaene (E186-E187) Compounds were prepared using procedures similar to those used for preparing E12, starting from D33 and 1-(tetrahydro- 2H-pyran-4-yl)ethyl methanesulfonate. E186: LC-MS: 393.3 [M + H]⁺. ¹H NMR (400 MHz, CDCl₃): δ 7.83 (s, 1H), 6.11 (s, 1H), 5.55 (t, J = 6.0 Hz, 1H), 4.40 (t, J = 4.8 Hz, 2H), 4.03 (dd, J = 11.2, 3.6 Hz, 1H), 3.91 (d, J = 12.8 Hz, 1H), 3.78~3.68 (m, 1H), 3.57~3.47 (m, 2H), 3.45~3.25 (m, 2H), 2.19 (s, 3H), 2.13~2.04 (m, 1H), 1.99~1.84 (m, 2H), 1.78~1.74 (m, 1H), 1.44 (d, J = 6.8 Hz, 3H), 1.42~1.35 (m, 1H), 1.19~1.10 (m, 2H). Chiral RT = 4.333 min (chiral method E); ee = 100%. E187: LC-MS: 393.3 [M + H]⁺. ¹H NMR (400 MHz, CDCl₃): δ 7.83 (s, 1H), 6.14 (s, 1H), 5.56 (t, J = 6.0 Hz, 1H), 4.40 (t, J = 4.8 Hz, 2H), 4.03 (dd, J = 11.2, 3.6 Hz, 1H), 3.90 (d, J = 10.4 Hz, 1H), 3.79~3.69 (m, 1H), 3.57~3.46 (m, 2H), 3.44~3.28 (m, 2H), 2.19 (s, 3H), |
| | 2.13~2.04 (m, 1H), 1.98~1.86 (m, 2H), 1.78~1.74 (m, 1H), 1.44 (d, |
| | J = 6.4 Hz, 3H), 1.38~1.32 (m, 1H), 1.22~1.14 (m, 2H). Chiral RT = |
| | 5.538 min (chiral method E); ee = 100%. |
| 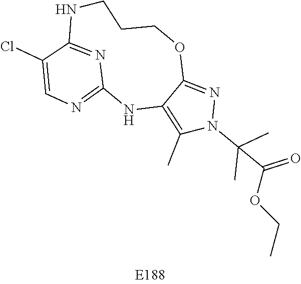 [SMILES: CCOC(=O)C(C)(C)n1nc2c(c1C)Nc1ncc(Cl)c(n1)NCCCO2] | 2-{14-Chloro-4-methyl-8-oxa-2,5,6,12,16,17-hexaazatricyclo [11.3.1.0^{3,7}]heptadeca-1(16),3,6,13(17),14-pentaen-5-yl}-2- methylpropanoate (E188) Compound was prepared using procedures similar to those used for preparing E37, starting from D33. LC-MS: 395.1 [M + H]⁺. ¹H NMR (400 MHz, CDCl₃): δ 7.80 (s, 1H), 6.21 (s, 1H), 5.54 (br, 1H), 4.39 (t, J = 4.4 Hz, 2H), 4.23 (q, J = 7.2 Hz, 2H), 3.55~3.51 (m, 2H), 2.10 (s, 3H), 1.91 (br, 2H), 1.76 (s, 6H), 1.27 (t, J = 7.0 Hz, 3H). |
| 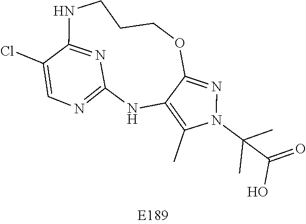 [SMILES: Cc1c2c(nn1C(C)(C)C(=O)O)OCCCNc1nc(ncc1Cl)N2] | 2-{14-Chloro-4-methyl-8-oxa-2,5,6,12,16,17-hexaazatricyclo [11.3.1.0^{3,7}]heptadeca-1(16),3,6,13(17),14-pentaen-5-yl}-2- methylpropanoic acid (E189) Compound was prepared using procedures similar to those used for preparing E39, starting from E188. LC-MS: 367.1 [M + H]⁺. ¹H NMR (400 MHz, DMSO-d₆): δ 13.07 (br, 1H), 8.33 (s, 1H), 7.77 (s, 1H), 7.36 (t, J = 5.6 Hz, 1H), 4.20 (br, 2H), 2.07(s, 3H), 1.71 (br, 2H), 1.62 (s, 6H). (2H overlapped). |
| 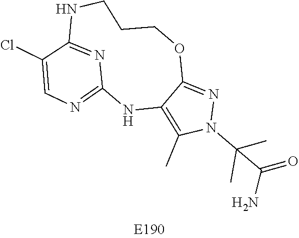 [SMILES: Cc1c2c(nn1C(C)(C)C(N)=O)OCCCNc1nc(ncc1Cl)N2] | 2-{14-Chloro-4-methyl-8-oxa-2,5,6,12,16,17-hexaazatricyclo [11.3.1.0^{3,7}]heptadeca-1(16),3,6,13(17),14-pentaen-5-yl}-2- methylpropanamide (E190) Compound was prepared using procedures similar to those used for preparing E41, starting from E189. LC-MS: 366.2 [M + H]⁺. ¹H NMR (400 MHz, DMSO-d₆): δ 8.32 (s, 1H), 7.77 (s, 1H), 7.32 (s, 1H), 7.26 (s, 1H), 7.02 (s, 1H), 4.23 (br, 2H), 2.07 (s, 3H), 1.73 (br, 2H), 1.56 (s, 6H). (2H overlapped). |
| 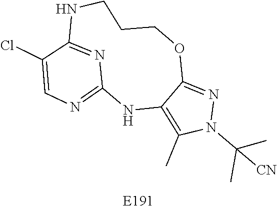 [SMILES: Cc1c2c(nn1C(C)(C)C#N)OCCCNc1nc(ncc1Cl)N2] | 2-{14-Chloro-4-methyl-8-oxa-2,5,6,12,16,17-hexaazatricyclo [11.3.1.0^{3,7}]heptadeca-1(16),3,6,13(17),14-pentaen-5-yl}-2- methylpropanenitrile (E191) Compound was prepared using procedures similar to those used for preparing E43, starting from E190. LC-MS: 348.2 [M + H]⁺. ¹H NMR (400 MHz, DMSO-d₆): δ 8.48 (s, 1H), 7.79 (s, 1H), 7.48 (s, 1H), 4.23 (br, 2H), 2.36 (s, 3H), 1.89 (s, 6H), 1.76 (br, 2H). (2H overlapped). |
| 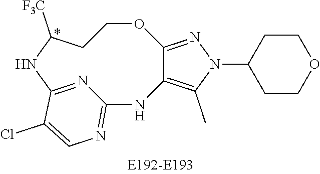 [SMILES: Cc1c2c(nn1C1CCOCC1)OCCC(C(F)(F)F)Nc1nc(ncc1Cl)N2] | Enantiomers 1-2: 14-Chloro-4-methyl-5-(oxan-4-yl)-11-(trifluoro methyl)-8-oxa-2,5,6,12,16,17-hexaazatricyclo[11.3.1.0^{3,7}] heptadeca-1(16),3,6,13(17),14-pentaene (E192-E193) Compound was prepared using procedures similar to those used for preparing E88, starting from 3-amino-4,4,4-trifluorobutan-1-ol. E192: LC-MS: 433.3 [M + H]⁺. ¹H NMR (400 MHz, CDCl₃): δ 7.91 (s, 1H), 6.54 (s, 1H), 5.42 (d, J = 8.0 Hz, 1H), 4.72~4.66 (m, 1H), 4.51~4.39 (m, 2H), 4.11~4.06 (m, 3H), 3.51 (t, J = 9.6 Hz, 2H), 2.36~2.23 (m, 3H), 2.23 (s, 3H), 1.85~1.76 (m, 3H). ¹⁹F NMR (376 MHz, CDCl₃): δ −75.66. Chiral RT = 2.935 min (chiral method A); ee = 100%. E193: LC-MS: 433.3 [M + H]⁺. ¹H NMR (400 MHz, CDCl₃): δ 7.91 (s, |
| | 1H), 6.46 (s, 1H), 5.37 (d, J = 8.4 Hz, 1H), 4.72~4.66 (m, 1H), |
| | 4.51~4.39 (m, 2H), 4.11~4.06 (m, 3H), 3.54~3.49 (m, 2H), 2.36~ |
| | 2.22 (m, 3H), 2.22 (s, 3H), 1.85~1.76 (m, 3H). ¹⁹F NMR (376 MHz, |
| | CDCl₃): δ −75.68. Chiral RT = 4.593 min (chiral method A); ee = |
| | 100%. |
| 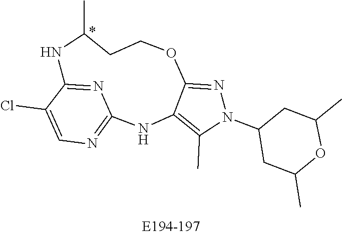 [SMILES: Cc1c2c(nn1C1CC(C)OC(C)C1)OCCC(C)Nc1nc(ncc1Cl)N2] | Enantiomers 1-4: 14-Chloro-5-(2,6-dimethyloxan-4-yl)-4,11- dimethyl-8-oxa-2,5,6,12,16,17-hexaazatricyclo[11.3.1.0^{3,7}] heptadeca-1(16),3,6,13(17),14-pentaene (E194-E197) Compounds were prepared using procedures similar to those used for preparing E79, starting from D73 and 2,6-dimethyltetrahydro-2H- pyran-4-yl methanesulfonate. E194: LC-MS: 407.2 [M + H]⁺. ¹H NMR (400 MHz, CDCl₃): δ 7.77 (s, 1H), 7.03 (br, 1H), 5.35 (d, J = 8.0 Hz, 1H), 4.49~4.43 (m, 2H), 4.34~4.26 (m, 3H), 4.05~4.03 (m, 1H), 2.20 (s, 3H), 1.92~1.82 (m, 3H), 1.68~1.60 (m, 3H), 1.28 (d, J = 8.0 Hz, 3H), 1.18 (dd, J = 4.0 Hz, 6H). Chiral RT = 3.054 min (chiral method F); ee = 100%. E195: LC-MS: 407.2 [M + H]⁺. ¹H NMR (400 MHz, CDCl₃): δ 7.81 (s, 1H), 6.22 (s, 1H), 5.15(d, J = 8.0 Hz, 1H), 4.49 (dd, J = 8.0 Hz, 1H), 4.33 (t, J = 8.0 Hz, 1H), 4.13~4.02 (m, 2H), 3.60~3.58 (m, 2H), 2.20 (s, 3H), 1.88~1.79 (m, 6H), 1.32 (d, J = 8.0 Hz, 3H), 1.27 (dd, J = 8.0 |
| | Hz, 6H). Chiral RT = 3.425 min (chiral method F); ee = 99.7%. |
| | E196: LC-MS: 407.2 [M + H]⁺. ¹H NMR (400 MHz, CDCl₃): δ 7.76 (s, |
| | 1H), 7.15 (br, 1H), 5.37 (d, J = 6.0 Hz, 1H), 4.49~4.42 (m, 2H), |
| | 4.44~4.26 (m, 3H), 4.04 (br, 1H), 2.20 (s, 3H), 1.96~1.82 (m, 4H), |
| | 1.72~1.60 (br, 2H), 1.35 (d, J = 8.0 Hz, 3H), 1.18 (dd, J = 6.0 Hz, |
| | 6H). Chiral RT = 4.066 min (chiral method F); ee = 100%. |
| | E197: LC-MS: 407.2 [M + H]⁺. ¹H NMR (400 MHz, CDCl₃): δ 7.78 (s, |
| | 1H), 6.66 (br, 1H), 5.25 (d, J = 8.0 Hz, 1H), 4.48 (dd, J = 8.0 Hz, 1H), |
| | 4.32 (t, J = 8.0 Hz, 1H), 4.13~4.02 (m, 2H), 3.61~3.57 (m, 2H), 2.21 |
| | (s, 3H), 1.93~1.79 (m, 6H), 1.33 (d, J = 8.0 Hz, 3H), 1.27 (t, J = 6.0 |
| | Hz, 6H). Chiral RT= 4.646 min (chiral method F); ee= 100%. |
| 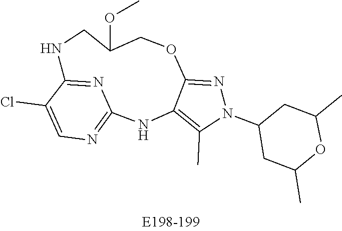 [SMILES: COC1CNc2nc(ncc2Cl)Nc2c(nn(C3CC(C)OC(C)C3)c2C)OC1] | Isomers 1-2: 14-Chloro-5-(2,6-dimethyloxan-4-yl)-10-methoxy-4- methyl-8-oxa-2,5,6,12,16,17-hexaazatricyclo[11.3.1.0^{3,7}] heptadeca-1(16),3,6,13(17),14-pentaene (E198-E199) Compounds were prepared using procedures similar to those used for preparing E79, using D13 and 2,6-dimethyltetrahydro-2H-pyran- 4-yl methanesulfonate as starting materials. E198: LC-MS: 423.2[M + H]⁺. ¹H NMR (400 MHz, CDCl₃): δ 7.84 (s, 1H), 6.18 (s, 1H), 5.71~5.68 (m, 1H), 4.50 (d, J = 10.0 Hz, 1H), 4.31 (t, J = 8.0 Hz, 1H), 4.13~4.07 (m, 1H), 3.93~3.88 (m, 1H), 3.61~3.57 (m, 2H), 3.41 (s, 3H), 3.34~3.27 (m, 2H), 2.19 (s, 3H), 1.90~1.76 (m, 4H), 1.27(t, J = 6.0 Hz, 6H). Chiral RT = 1.166 min (chiral method A); ee = 100%. E199: LC-MS: 423.2[M + H]⁺. ¹H NMR (400 MHz, CDCl₃): δ 7.83 (s, 1H), 6.33 (s, 1H), 5.75~5.73(m, 1H), 4.50 (d, J = 8.0 Hz, 1H), 4.24 (t, J = 6.0 Hz, 1H), 4.12~4.09 (m, 1H), 3.94~3.88 (m, 1H), 3.61~3.58 |
| | (m, 2H), 3.42 (s, 3H), 3.35~3.28 (m, 2H), 2.20 (s, 3H), 1.90~1.76 |
| | (m, 4H), 1.20 (t, J = 6.0 Hz, 6H). Chiral RT = 2.495 min (chiral |
| | method A); ee = 99.8%. |
| 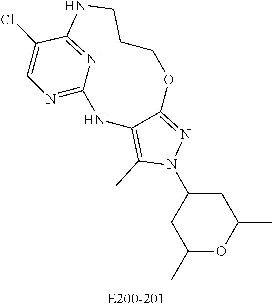 [SMILES: Cc1c2c(nn1C1CC(C)OC(C)C1)OCCCNc1nc(ncc1Cl)N2] | Enantiomers 1-2: 14-Chloro-5-(2,6-dimethyloxan-4-yl)-4-methyl- 8-oxa-2,5,6,12,16,17-hexaazatricyclo[11.3.1.0^{3,7}]heptadeca- 1(16),3,6,13(17),14-pentaene (E200-E201) Compounds were prepared using procedures similar to those used for preparing E12, starting from D33 and 2,6-dimethyltetrahydro-2H- pyran-4-yl methanesulfonate. E200: LC-MS: 393.2 [M + H]⁺. ¹H NMR (400 MHz, CDCl₃): δ 7.82 (s, 1H), 6.12 (s, 1H), 5.54 (t, J = 5.6 Hz, 1H), 4.42~4.34 (m, 5H), 3.52 (dd, J = 10.6, 5.8 Hz, 2H), 2.18 (s, 3H), 1.93 (br, 2H), 1.89~1.86 (m, 2H), 1.68~1.60 (m, 2H), 1.18 (d, J = 6.4 Hz, 6H). Chiral RT = 5.313 min (chiral method A); ee = 100%. E201: LC-MS: 393.2 [M + H]⁺. ¹H NMR (400 MHz, CDCl₃): δ 7.80 (s, 1H), 6.33 (s, 1H), 5.54 (t, J = 6.0 Hz, 1H), 4.40 (t, J = 4.4 Hz, 2H), 4.13~4.07 (m, 1H), 3.61~3.58 (m, 2H), 3.54~3.49 (m, 2H), 2.20 (s, 3H), 1.91~1.79 (m, 6H), 1.27 (d, J = 6.0 Hz, 6H). Chiral RT = 6.132 min (chiral method A); ee = 100%. |
| 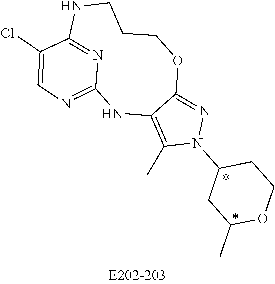 [SMILES: Cc1c2c(nn1C1CCOC(C)C1)OCCCNc1nc(ncc1Cl)N2] | Enantiomers 1-2: 14-Chloro-4-methyl-5-(2-methyloxan-4-yl)-8- oxa-2,5,6,12,16,17-hexaazatricyclo[11.3.1.0^{3,7}]heptadeca- 1(16),3,6,13(17),14-pentaene (E202-E203) Compounds were prepared using procedures similar to those used for preparing E12, starting from D33 and 2-methyltetrahydro-2H- pyran-4-yl methanesulfonate. E202: LC-MS: 379.2 [M + H]⁺. ¹H NMR (400 MHz, CDCl₃): δ 7.79 (s, 1H), 6.61 (s, 1H), 5.61 (t, J = 5.6 Hz, 1H), 4.39 (t, J = 4.4 Hz, 2H), 4.33~4.30 (m, 1H), 4.24~4.19 (m, 1H), 3.86~3.82 (m, 1H), 3.53 (dd, J = 10.8, 5.6 Hz, 2H), 2.20 (s, 3H), 2.03~1.94 (m, 5H), 1.75~1.70 (m, 1H), 1.20 (d, J = 6.4 Hz, 3H). Chiral RT = 2.788 min (chiral method C); ee = 100%. E203: LC-MS: 379.2 [M + H]⁺. ¹H NMR (400 MHz, CDCl₃): δ 7.79 (s, 1H), 6.61 (s, 1H), 5.64 (t, J = 5.6 Hz, 1H), 4.39 (t, J = 4.4 Hz, 2H), 4.33~4.30 (m, 1H), 4.23~4.19 (m, 1H), 3.85~3.82 (m, 1H), 3.53 (q, J = 5.6 Hz, 2H), 2.20 (s, 3H), 2.03~1.93 (m, 5H), 1.74~1.69 (m, 1H), 1.20 (d, J = 6.4 Hz, 3H). Chiral RT = 2.963 min (chiral method C); ee = 100%. |
| 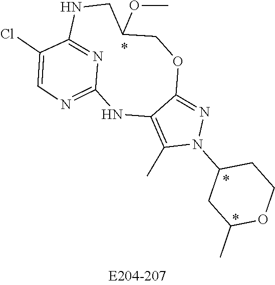 [SMILES: COC1CNc2nc(ncc2Cl)Nc2c(nn(C3CCOC(C)C3)c2C)OC1] | Enantiomers 1-4: 14-Chloro-10-methoxy-4-methyl-5-(2-methyl oxan-4-yl)-8-oxa-2,5,6,12,16,17-hexaazatricyclo [11.3.1.0^{3,7}] heptadeca-1(16),3,6,13(17),14-pentaene (E204-E207) Compounds were prepared using procedures similar to those used for preparing E12, using D13 and 2-methyltetrahydro-2H-pyran-4-yl methanesulfonate as starting materials. E204: LC-MS: 409.3 [M + H]⁺. ¹H NMR (400 MHz, CDCl₃): δ 7.84 (s, 1H), 6.12 (s, 1H), 5.68 (br, 1H), 4.49 (d, J = 12.0 Hz, 1H), 4.39 (t, J = 4.0 Hz, 1H), 4.31 (t, J = 12.0 Hz, 1H), 4.20~4.08 (m, 1H), 3.95~3.90 (m, 1H), 3.86~3.81 (m, 1H), 3.55~3.51 (m, 1H), 3.44 (d, J = 12.0 Hz, 3H), 3.36~3.27 (m, 2H), 2.18 (t, J = 4.0 Hz, 3H), 2.04~2.01 (m, 1H), 1.90~1.86 (m, 1H), 1.75~1.68 (m, 1H), 1.26 (t, J = 12.0 Hz, 2H), 1.20 (d, J = 6.0 Hz, 3H), (Chiral RT = 2.282 min (chiral method C); ee = 100%. E205: LC-MS: 409.2 [M + H]⁺. ¹H NMR (400 MHz, CDCl₃): δ 7.84 (s, 1H), 6.12 (s, 1H), 5.69 (br, 1H), 4.48 (d, J = 12.0 Hz, 1H), 4.40 (t, J = 8.0 Hz, 1H), 4.31 (t, J = 12.0 Hz, 2H), 4.18 (t, J = 8.0 Hz, 1H), 3.91~ 3.88 (m, 1H), 3.85~3.82 (m, 1H), 3.43 (s, 3H), 3.36~3.26 (m, 2H), 2.18 (d, J = 4.0 Hz, 3H), 2.03~1.91 (m, 3H), 1.74~1.67 (m, 1H), 1.19 |
| | (d, J = 8.0 Hz, 3H). Chiral RT = 2.390 min (chiral method C); |
| | ee = 95%. |
| | E206: LC-MS: 409.2 [M + H]⁺. ¹H NMR (400 MHz, CDCl₃): δ 7.84 (s, |
| | 1H), 6.18 (s, 1H), 5.69 (br, 1H), 4.49 (d, J = 12.0 Hz, 1H), 4.39 (t, J = |
| | 4.0 Hz, 1H), 4.31 (t, J = 8.0 Hz, 1H), 4.20~4.08 (m, 1H), 3.94~3.829 |
| | (m, 1H), 3.85~3.82 (m, 1H), 3.54 (t, J = 12.0 Hz, 1H), 3.43 (d, J = 4.0 |
| | Hz, 3H), 3.36~3.27 (m, 2H), 2.18 (t, J = 8.0 Hz, 3H), 2.04~1.98 (m, |
| | 1H), 1.90~1.81 (m, 1H), 1.75~1.68 (m, 1H), 1.25 (t, J = 6.0 Hz, 2H), 1.20 (d, J = 13.2 Hz, 2H). Chiral RT = 2.618 min (chiral method C); |
| | ee = 99.3%. |
| | E207: LC-MS: 409.2 [M + H]⁺. ¹H NMR (400 MHz, CDCl₃): δ 7.84 (s, |
| | 1H), 6.21 (s, 1H), 5.71 (br, 1H), 4.48 (d, J = 12.0 Hz, 1H), 4.40 (t, J = |
| | 4.0 Hz, 1H), 4.31 (t, J = 8.0 Hz, 1H), 4.18 (t, J = 8.0 Hz, 1H), 3.94~3.89 |
| | (m, 1H), 3.86~3.82 (m, 1H), 3.43 (s, 3H), 3.36~3.27 (m, 2H), |
| | 2.18 (d, J = 4.0 Hz, 3H), 2.04~1.90 (m, 3H), 1.74~1.67 (m, 1H), 1.19 |
| | (d, J = 8.0 Hz, 3H). Chiral RT = 2.798 min (chiral method C); ee = |
| | 98.6%. |
| 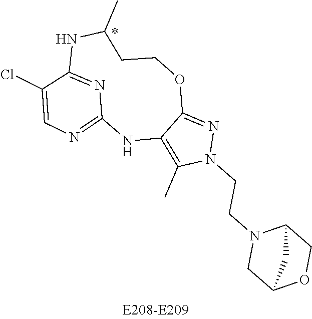 [SMILES: Cc1c2c(nn1CCN1C[C@H]3C[C@@H]1CO3)OCCC(C)Nc1nc(ncc1Cl)N2] | Enantiomers 1-2: 14-Chloro-4,11-dimethyl-5-{2-[(1S,4S)-2-oxa- 5-azabicyclo[2.2.1]heptan-5-yl]ethyl}-8-oxa-2,5,6,12,16,17-hexa azatricyclo[11.3.1.0^{3,7}]heptadeca-1(16),3,6,13(17),14-pentaene (E208-E209) To a solution of D163 (270 mg, 0.8 mmol) in MeOH (10 mL) was added (1S,4S)-2-oxa-5-azabicyclo[2.2.1]heptane hydrochloride (240 mg, 2.4 mmol). After stirred for 30 min, NaBH₃CN (151 mg, 2.4 mmol) was added. The reaction was stirred overnight at room temperature. The mixture was poured into the sat.NaCl (aq) and extracted with EtOAc (3 × 100 mL). The combined organic layer was washed with brine, dried over anhydrous Na₂SO₄ and concentrated. The crude was poured into MeCN (2 mL) and filtered. The filter cake was dried to give the racemic, which was further separated by chiral HPLC to give the title compounds as off-white solids. E208: LC-MS: 420.2 [M + H]⁺. ¹H NMR (400 MHz, CDCl₃): δ 7.82 (s, 1H), 6.22 (br, 1H), 5.14 (d, J = 6.8 Hz, 1H), 4.47 (dd, J = 7.2 Hz, 3.2 Hz, 1H), 4.40 (br, 1H), 4.32 (t, J = 10 Hz, 1H), 4.03~4.01 (m, 4H), 3.62 (d, J = 6.0 Hz, 1H), 3.46 (br, 1H), 3.04~3.02 (m, 2H), 2.94 (d, J = 9.6 Hz, 1H), 2.61 (d, J = 10.0 Hz, 1H), 2.23 (s, 3H), 1.89~1.75 (m, 4H), 1.32 (d, J = 7.2 Hz, 3H). Chiral RT = 6.260 min (chiral method G); ee = 100%. |
| | E209: LC-MS: 420.2 [M + H]⁺. ¹H NMR (400 MHz, CDCl₃): δ 7.82 (s, |
| | 1H), 6.20 (br, 1H), 5.27 (d, J = 7.2 Hz, 1H), 4.48~4.45 (m, 1H), 4.39 |
| | (br, 1H), 4.33 (t, J = 10.4 Hz, 1H), 4.03~4.01 (m, 4H), 3.60 (d, J = 6.8 |
| | Hz, 1H), 3.41 (br, 1H), 3.06~3.01 (m, 2H), 2.91 (d, J = 9.6 Hz, 1H), |
| | 2.80 (d, J = 9.6 Hz, 1H), 2.22 (s, 3H), 1.93~1.73 (m, 4H), 1.31 (d, J = |
| | 6.8 Hz, 3H). Chiral RT = 9.738 min (chiral method G); ee = 100%. |
| 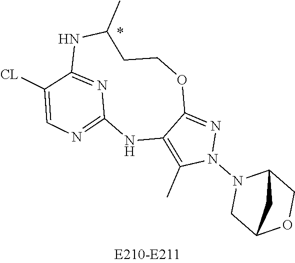 | Enantiomers 1-2: 14-Chloro-4,11-dimethyl-5-{2-[(1R,4R)-2-oxa- 5-azabicyclo[2.2.1]heptan-5-yl]ethyl}-8-oxa-2,5,6,12,16,17-hexa azatricyclo[11.3.1.0^{3,7}]heptadeca-1(16),3,6,13(17),14-pentaene (E210-E211) Compounds were prepared using procedures similar to those used for preparing E37, starting from (1R,4R)-5-(2-chloroethyl)-2-oxa-5- azabicyclo[2.2.1] heptane and D73. E210: LC-MS: 420.2 [M + H]⁺. ¹H NMR (400 MHz, CDCl₃): δ 7.81 (s, 1H), 6.31 (br, 1H), 5.14 (d, J = 6.8 Hz, 1H), 4.49~4.46 (m, 1H), 4.39 (s, 1H), 4.32 (t, J = 10.4 Hz, 1H), 4.00 (d, J = 7.6 Hz, 4H), 3.62 (d, J = 8.0 Hz, 1H), 3.42 (br, 1H), 3.00~2.97 (m, 2H), 2.91 (d, J = 10.0 Hz, 1H), 2.58 (d, J = 10.0 Hz, 1H), 2.22 (s, 3H), 1.87~1.75 (m, 4H), 1.31 (d, J = 7.2 Hz, 3H). Chiral RT = 6.640 min (chiral method G); ee = 100%. E211: LC-MS: 420.2 [M + H]⁺. ¹H NMR (400 MHz, CDCl₃): δ 7.82 (s, 1H), 6.20 (br, 1H), 5.13 (d, J = 6.8 Hz, 1H), 4.49~4.48 (m, 1H), 4.39 (s, 1H), 4.36 (t, J = 10.8 Hz, 1H), 4.10 (d, J = 7.2 Hz, 4H), 3.61 (d, J = 7.6 Hz, 1H), 3.40 (br, 1H), 3.03~3.00 (m, 2H), 2.93 (d, J = 9.2 Hz, |
| | 1H), 2.57 (d, J = 9.2 Hz, 1H), 2.22 (s, 3H), 1.89~1.73 (m, 4H), 1.31 |
| | (d, J = 6.8 Hz, 3H). Chiral RT = 9.399 min (chiral method G); |
| | ee = 100% |
| 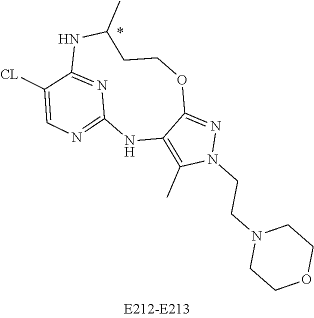 | Enantiomers 1-2: 14-Chloro-4,11-dimethyl-5-[2-(morpholin-4- yl)ethyl]-8-oxa-2,5,6,12,16,17-hexaazatricyclo[11.3.1.0^{3,7}] heptadeca-1(16),3,6,13(17),14-pentaene (E212-E213) Compounds were prepared using procedures similar to those used for preparing E37, starting from 4-(2-chloroethyl)morpholine and D73. E212: LC-MS: 408.3 [M + H]⁺. ¹H NMR (400 MHz, CDCl₃): δ 7.81 (s, 1H), 6.16 (s, 1H), 5.11 (dd, J = 4.0 Hz, 1H), 4.47 (d, J = 12.0 Hz, 1H), 4.32 (t, J = 8.0 Hz, 1H), 4.04~3.97 (m, 3H), 3.68 (t, J = 4.0 Hz, 4H), 2.72 (t, J = 4.0 Hz, 2H), 2.53~2.44 (m, 4H), 2.20 (s, 3H), 1.88~1.78 (br, 2H), 1.31 (d, J = 8.0 Hz, 3H). Chiral RT = 3.364 min (chiral method A); ee = 100%. E213: LC-MS: 408.3 [M + H]⁺. ¹H NMR (400 MHz, CDCl₃): δ 7.81 (s, 1H), 6.16 (s, 1H), 5.11 (d, J = 4.0 Hz, 1H), 4.47 (dd, J = 12.0 Hz, 1H), 4.32 (t, J = 8.0 Hz, 1H), 4.04~3.97 (m, 3H), 3.68 (t, J = 4.0 Hz, 4H), 2.72 (t, J = 4.0 Hz, 2H), 2.53~2.44 (m, 4H), 2.20 (s, 3H), 1.88~1.78 (s, 2H), 1.31 (d, J = 8.0 Hz, 3H). Chiral RT = 5.119 min (chiral method A); ee = 99.1%. |
| 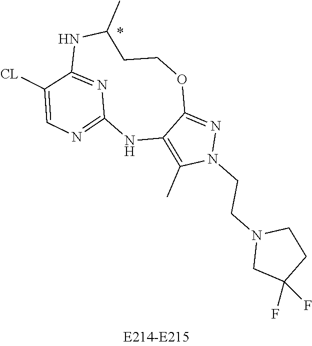 | Enantiomers 1-2: 14-Chloro-5-[2-(3,3-difluoropyrrolidin-1-yl) ethyl]-4,11-dimethyl-8-oxa-2,5,6,12,16,17-hexaazatricyclo [11.3.1.0^{3,7}]heptadeca-1(16),3,6,13(17),14-pentaene (E214-E215) Compounds were prepared using procedures similar to those used for preparing E208, starting from D163 and 3,3-difluoropyrrolidine. E214: LC-MS: 428.1 [M + H]⁺. ¹H NMR (400 MHz, CDCl₃): δ 7.79 (s, 1H), 6.70 (br, 1H), 5.25 (d, J = 6.4 Hz, 1H), 4.49~4.45 (m, 1H), 4.32 (t, J = 10.8 Hz, 1H), 4.03~3.97 (m, 3H), 2.91~2.84 (m, 4H), 2.76 (d, J = 6.8 Hz, 2H), 2.28~2.21 (m, 2H), 2.20 (s, 3H), 1.90~1.84 (m, 2H), 1.32 (d, J = 6.8 Hz, 3H). ¹⁹F NMR (400 MHz, CDCl₃): δ −92.77 (s, 2F). Chiral RT = 2.370 min (chiral method A); ee = 100%. E215: LC-MS: 428.1 [M + H]⁺. ¹H NMR (400 MHz, CDCl₃): δ 7.80 (s, 1H), 6.53 (br, 1H), 5.19 (d, J = 7.2 Hz, 1H), 4.49~4.45 (m, 1H), 4.33 (t, J = 10.8 Hz, 1H), 4.03~3.97 (m, 3H), 2.94~2.84 (m, 4H), 2.76 (d, J = 6.8 Hz, 2H), 2.30~2.23 (m, 2H), 2.20 (s, 3H), 1.89~1.79 (m, 2H) 1.32 (d, J = 7.2 Hz, 3H). ¹⁹F NMR (400 MHz, CDCl₃): δ −92.77 (s, 2F). Chiral RT = 2.370 min (chiral method A); ee = 100%. |
| 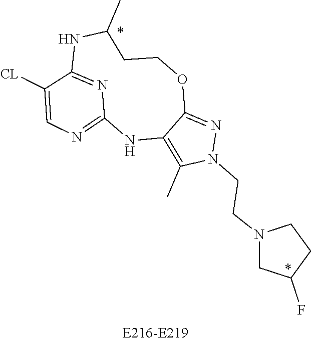 | Enantiomers 1-4: 14-Chloro-5-[2-(3-fluoropyrrolidin-1-yl)ethyl]- 4,11-dimethyl-8-oxa-2,5,6,12,16,17-hexaazatricyclo [11.3.1.0^{3,7}] heptadeca-1(16),3,6,13(17),14-pentaene (E216-E219) Compounds were prepared using procedures similar to those used for preparing E208, starting from D163 and 3-fluoropyrrolidine. E216: LC-MS: 410.1 [M + H]⁺. ¹H NMR (400 MHz, CDCl₃): δ 7.81 (s, 1H), 6.33 (br, 1H), 5.23~5.08 (m, 2H), 4.48~4.45 (m, 1H), 4.33 (t, J = 10.4 Hz, 1H), 4.11~4.00 (m, 3H), 3.01~2.84 (m, 5H), 2.64 (br, 1H), 2.23 (s, 3H), 2.18~2.07 (m, 2H), 1.89~1.83 (m, 2H), 1.31 (d, J = 6.8 Hz, 3H). ¹⁹F NMR (400 MHz, CDCl₃): δ −168.59 (m, 1F). Chiral RT = 3.145 min (chiral method A); ee = 100%. E217: LC-MS: 410.2 [M + H]⁺. ¹H NMR (400 MHz, CDCl₃): δ 7.81 (s, 1H), 6.46 (br, 1H), 5.25~5.12 (m, 2H), 4.48~4.45 (m, 1H), 4.32 (t, J = 10.4 Hz, 1H), 4.16~4.02 (m, 3H), 3.06~2.89 (m, 5H), 2.74 (br, 1H), 2.24 (s, 2H), 2.20~2.09 (m, 2H), 1.93~1.79 (m, 2H), 1.32 (d, J = 6.8 Hz, 3H). ¹⁹F NMR (400 MHz, CDCl₃): δ −168.59 (m, 1F). Chiral RT = 3.495 min (chiral method A); ee = 100%. E218: LC-MS: 410.2 [M + H]⁺. ¹H NMR (400 MHz, CDCl₃): δ 7.81 (s, 1H), 6.32 (br, 1H), 5.23~5.10 (m, 2H), 4.47 (dd, J = 10.8, 3.2 Hz, 1H), 4.33 (t, J = 10.8 Hz, 1H), 4.10~4.02 (m, 3H), 3.00~2.80 (m, 5H), 2.59 (br, 1H), 2.22 (s, 3H), 2.20~2.03 (m, 2H), 1.93~1.79 (m, 2H), J = 6.8 Hz, 2H), 2.30~2.23 (m, 2H), 2.20 (s, 3H), 1.89~1.79 (m, 2H) |
| | 1.32 (d, J = 6.8 Hz, 3H). ¹⁹F NMR (400 MHz, CDCl₃): δ −168.59 (m, |
| | 1F). Chiral RT = 6.121 min (chiral method A); 6.084 min (chiral |
| | method E); ee = 100%. |
| | E219: LC-MS: 410.2 [M + H]⁺. ¹H NMR (400 MHz, CDCl₃): δ 7.81 (s, |
| | 1H), 6.34 (br, 1H), 5.23~5.09 (m, 2H), 4.47 (dd, J = 11.6, 2.8 Hz, |
| | 1H), 4.33 (t, J = 10.8 Hz, 1H), 4.12~4.00 (m, 3H), 3.01~2.84 (m, 5H), |
| | 2.65 (br, 1H), 2.23 (s, 3H), 2.20~2.05 (m, 2H), 1.89~1.79 (m, 2H), |
| | 1.32 (d, J = 6.8 Hz, 3H). ¹⁹F NMR (400 MHz, CDCl₃): δ −168.59 (m, |
| | 1F). Chiral RT = 6.121 min (chiral method A), 7.151 min (chiral |
| | method E); ee = 99.6%. |
| 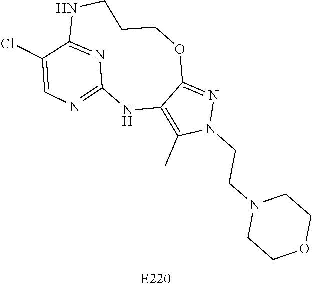 [SMILES: Cc1c2c(nn1CCN1CCOCC1)OCCCNc1nc(ncc1Cl)N2] | 14-Chloro-4-methyl-5-[2-(morpholin-4-yl)ethyl]-8-oxa-2,5,6,12, 16,17-hexaazatricyclo[11.3.1.0^{3,7}]heptadeca-1(16),3,6,13(17),14- pentaene (E220) Compound was prepared using procedures similar to those used for preparing E12, starting from D33 and 4-(2-chloroethyl)morpholine hydrochloride. LC-MS: 394.2 [M + H]⁺. ¹H NMR (400 MHz, CDCl₃): δ 7.82 (s, 1H), 6.18 (s, 1H), 5.55 (s, 1H), 4.38 (t, J = 4.4 Hz, 2H), 4.00 (t, J = 6.8 Hz, 2H), 3.69 (t, J = 4.4 Hz, 4H), 3.51 (dd, J = 10.8, 6.0 Hz, 2H), 2.72 (t, J = 6.8 Hz, 2H), 2.48 (t, J = 4.2 Hz, 4H), 2.21 (s, 3H), 1.92 (br, 2H). |
| 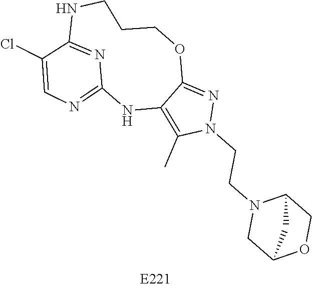 [SMILES: Cc1c2c(nn1CCN1C[C@H]3C[C@@H]1CO3)OCCCNc1nc(ncc1Cl)N2] | 14-Chloro-4-methyl-5-{2-[(1S,4S)-2-oxa-5-azabicyclo[2.2.1] heptan-5-yl]ethyl}-8-oxa-2,5,6,12,16,17-hexaazatricyclo [11.3.1.0^{3,7}]heptadeca-1(16),3,6,13(17),14-pentaene (E221) Compound was prepared using procedures similar to those used for preparing E12, starting from D33 and (1S,4S)-5-(2-chloroethyl)-2- oxa-5-azabicyclo[2.2.1]heptane. LC-MS: 406.4 [M + H]⁺. ¹H NMR (400 MHz, CDCl₃): δ 7.82 (s, 1H), 6.07 (s, 1H), 5.55~5.53 (m, 1H), 4.40~4.37 (m, 3H), 3.97~3.94 (m, 3H), 3.60 (d, J = 8.0 Hz, 1H), 3.51 (q, J = 5.6 Hz, 2H), 3.37 (br, 1H), 2.98~2.90 (m, 2H), 2.87 (d, J = 9.6 Hz, 1H), 2.53 (d, J = 10.0 Hz 1H), 2.21 (s, 3H), 1.93~1.91 (m, 2H), 1.80 (d, J = 10.0 Hz, 1H), 1.71 (d, J = 9.2 Hz, 1H). |
| 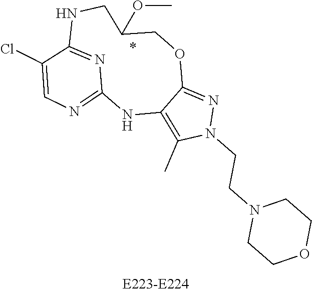 [SMILES: COC1CNc2nc(ncc2Cl)Nc2c(nn(CCN3CCOCC3)c2C)OC1] | Enantiomers 1-2: 14-Chloro-10-methoxy-4-methyl-5-[2- (morpholin-4-yl)ethyl]-8-oxa-2,5,6,12,16,17-hexaazatricyclo [11.3.1.0^{3,7}]heptadeca-1(16),3,6,13(17),14-pentaene (E223-E224) Compounds were prepared using procedures similar to those used for preparing E12 using D13 and 4-(2-chloroethyl)morpholine hydrochloride as starting materials. E223: LC-MS: 424.2 [M + H]⁺. ¹H NMR (400 MHz, CDCl₃): δ 7.82 (s, 1H), 6.79 (s, 1H), 5.85 (br, 1H), 4.46 (d, J = 12.0 Hz, 2H), 4.28 (t, J = 8.0 Hz, 1H), 4.13~4.04 (m, 3H), 3.92~3.88 (m, 1H), 3.74~3.72 (m, 4H), 3.42 (s, 3H), 3.32 (d, J = 8.0 Hz, 2H), 2.84~2.83 (m, 2H), 2.78~ 2.70 (m, 3H), 2.24 (s, 3H). Chiral RT = 1.171 min (chiral method A); ee = 100%. E224: LC-MS: 410.1 [M + H]⁺. ¹H NMR (400 MHz, CDCl₃): δ 7.82 (s, 1H), 6.79 (s, 1H), 5.85 (br, 1H), 4.46 (d, J = 12.0 Hz, 1H), 4.30 (t, J = 8 Hz, 1H), 4.11~4.05 (m, 3H), 3.93~3.88 (m, 1H), 3.74~3.72 (m, 4H), 3.42 (s, 3H), 3.32 (d, J = 8.0 Hz, 2H), 2.84~2.81 (m, 2H), 2.56~ 2.55 (m, 3H), 2.24 (s, 3H). Chiral RT = 1.520 min (chiral method A); ee = 100%. |
| 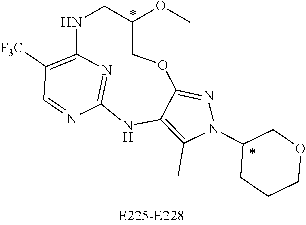 [SMILES: COC1CNc2nc(ncc2C(F)(F)F)Nc2c(nn(C3CCCOC3)c2C)OC1] | Enantiomers 1-4: 10-Methoxy-4-methyl-5-(oxan-3-yl)-14- (trifluoro methyl)-8-oxa-2,5,6,12,16,17-hexaazatricyclo [11.3.1.0^{3,7}] heptadeca-1(17),3,6,13,15-pentaene (E225-E228) Compounds were prepared using procedures similar to those used for preparing E2, using D25, D13 and 2,4-dichloro-5- (trifluoromethyl)pyrimidine as starting materials. E225: LC-MS: 429.2 [M + H]⁺. ¹H NMR (400 MHz, CDCl₃): δ 8.09 (s, 1H), 6.92 (br, 1H), 5.80 (br, 1H), 4.47 (d, J = 8.0 Hz, 1H), 4.32 (t, J = 12.0 Hz, 1H), 4.08~3.88 (m, 4H), 3.65 (t, J = 8.0 Hz, 1H), 3.64~3.63 (m, 1H), 3.61 (s, 3H), 3.45 (d, J = 8.0 Hz, 1H), 3.26 (d, J = 8.0 Hz, 1H), 2.31~2.2.30 (m, 1H), 2.22 (s, 3H), 2.07~2.04 (m, 1H), 1.83~1.78 (m, 2H). ¹⁹F NMR (400 MHz, CDCl₃): δ −61.03 (s, 3F). Chiral RT = 2.730 min (chiral method A); ee = 100%. E226: LC-MS: 429.3 [M + H]⁺. ¹H NMR (400 MHz, CDCl₃): δ 8.07 (s, 1H), 7.25 (br, 1H), 5.80 (br, 1H), 4.46 (d, J = 12.0 Hz, 1H), 4.28 (t, J = |
| | 8.0 Hz, 1H), 4.04~3.95 (m, 4H), 3.72 (t, J = 8.0 Hz, 1H), 3.44~ |
| | 3.43 (m, 1H), 3.41 (s, 3H), 3.35~3.26 (m, 2H), 2.22 (s, 3H), 2.17~ |
| | 2.00 (m, 2H), 1.83~1.78 (m, 2H). ¹⁹F NMR (400 MHz, CDCl₃): δ −61.03 |
| | (s, 3F). Chiral RT = 2.730 min (chiral method A); ee = 98.9%. |
| | E227: LC-MS: 429.3 [M + H]⁺. ¹H NMR (400 MHz, CDCl₃): δ 8.07 (s, |
| | 1H), 7.27 (br, 1H), 5.89 (br, 1H), 4.46 (d, J = 12.0 Hz, 1H), 4.28 (t, J = |
| | 12 Hz, 1H), 4.04~3.95 (m, 4H), 3.72 (t, J = 12.0 Hz, 1H), |
| | 3.44~3.43 (m, 1H), 3.41 (s, 3H), 3.35~3.30 (m, 2H), 2.22 (s, 3H), |
| | 2.17~2.12 (m, 1H), 2.04~2.00 (m, 1H), 1.80~1.78 (m, 2H), ¹⁹F NMR |
| | (400 MHz, CDCl₃): δ −61.03 (s, 3F). Chiral RT = 3.254 min (chiral |
| | method A); ee = 97.6%. |
| | E228: LC-MS: 429.2 [M + H]⁺. ¹H NMR (400 MHz, CDCl₃): δ 7.97 (s, |
| | 1H), 7.21 (br, 1H), 6.39 (br, 1H), 4.46 (d, J = 12.0 Hz, 1H), 4.28 (t, |
| | J = 12.0 Hz, 1H), 4.05~3.87 (m, 4H), 3.61 (t, J = 12.0 Hz, 1H), 3.43 |
| | (s, 3H), 3.39~3.31 (m, 3H), 2.28 (s, 3H), 2.25~2.21 (m, 1H), |
| | 2.07~2.00 (m, 1H), 1.81~1.77 (m, 2H). ¹⁹F NMR (400 MHz, CDCl₃): |
| | δ −61.03 (s, 3 F). Chiral RT = 3.644 min (chiral method A); ee = 98.9%. |
| 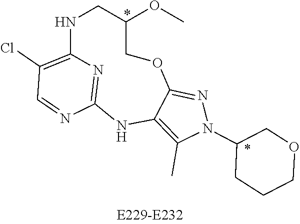 [SMILES: COC1CNc2nc(ncc2Cl)Nc2c(nn(C3CCCOC3)c2C)OC1] | Enantiomers 1-4: 14-Chloro-10-methoxy-4-methyl-5-(oxan-3-yl)- 8-oxa-2,5,6,12,16,17-hexaazatricyclo[11.3.1.0^{3,7}]heptadeca- 1(17),3,6,13,15-pentaene (E229-E232) Compounds were prepared using procedures similar to those used for preparing E2, using D25 and D13 as starting materials. E229: LC-MS: 395.2 [M + H]⁺. ¹H NMR (400 MHz, CDCl₃): δ 7.81 (s, 1H), 7.24 (br, 1H), 5.85 (br, 1H), 4.48 (d, J = 12.0 Hz, 2H), 4.26 (t, J = 8.0 Hz, 1H), 4.06~3.87 (m, 4H), 3.72 (t, J = 8.0 Hz, 1H), 3.46~3.43 (m, 1H), 3.42 (s, 3H), 3.38~3.31 (m, 2H), 2.21 (s, 3H), 2.18~2.10 (m, 1H), 1.92~1.92 (m, 1 H), 1.82~1.80 (m, 2H). Chiral RT = 4.202 min (chiral method A); ee = 100%. E230: LC-MS: 395.2 [M + H]⁺. ¹H NMR (400 MHz, CDCl₃): δ 7.80 (s, 1H), 6.95 (br, 1H), 5.90 (br, 1H), 4.47 (d, J = 12.0 Hz, 1H), 4.29 (t, J = 8.0 Hz, 1H), 4.07~3.88 (m, 4H), 3.62 (t, J = 8.0 Hz, 1H), 3.46~3.45 (m, 1H), 3.42 (s, 3H), 3.36~3.33 (m, 2H), 2.28~2.25 (m, 1H), 2.22 |
| | (s, 3H), 2.07~2.04 (m, 1 H), 1.83~1.78 (m, 2H), Chiral RT = 4.381 min |
| | (chiral method A); ee = 99.1%. |
| | E231: LC-MS: 395.2 [M + H]⁺. ¹H NMR (400 MHz, CDCl₃): δ 11.41 (s, |
| | 1H), 7.68 (s, 1H), 6.60 (br, 1H), 4.47 (d, J = 12.0 Hz, 1H), 4.29 (t, J = |
| | 8.0 Hz, 1H), 4.06~3.88 (m, 1H), 3.96~3.88 (m, 3H), 3.60 (t, J = |
| | 8.0 Hz, 1H), 3.48~3.47 (m, 1H), 3.44 (s, 3H), 3.32~3.30 (m, 2H), |
| | 2.28 (s, 3H), 2.25~2.21 (m, 1H), 2.06~2.03 (m, 1 H), 1.83~1.78 (m, |
| | 2H). Chiral RT = 4.958 min (chiral method A); ee = 99.7%. |
| | E232: LC-MS: 395.2 [M + H]⁺. ¹H NMR (400 MHz, CDCl₃): δ 7.79 (s, |
| | 1H), 7.02 (br, 1H), 5.90 (br, 1H), 4.48 (d, J = 8.0 Hz, 1H), 4.26 (t, J = |
| | 8.0 Hz, 1H), 4.06~3.88 (m, 4H), 3.72 (t, J = 8.0 Hz, 1H ), 3.46~3.44 |
| | (m, 1H), 3.42 (s, 3H), 3.34 (d, J = 12.0 Hz, 2H), 2.22 (s, 3H), |
| | 2.16~2.08 (m, 1H), 2.07~2.00 (m, 1 H), 1.93~1.80 (m, 2H). Chiral |
| | RT = 5.157 min (chiral method A); ee = 99.6%. |
| 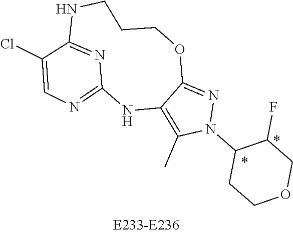 [SMILES: Cc1c2c(nn1C1CCOCC1F)OCCCNc1nc(ncc1Cl)N2] | Enantiomers 1-4: 14-Chloro-5-(3-fluorooxan-4-yl)-4-methyl-8- oxa-2,5,6,12,16,17-hexaazatricyclo[11.3.1.0^{3,7}]heptadeca- 1(16),3,6,13 (17),14-pentaene (E233-E236) Compounds were prepared using procedures similar to those used for preparing E21, starting from D50 and tert-butyl (3- bromopropyl)carbamate. E233: LC-MS: 383.1 [M + H]⁺. ¹H NMR (400 MHz, CD₃OD): δ 7.70 (s, 1H), 4.85~4.71 (m, 1H) 4.37~4.32 (m, 3H) 4.19~4.15 (q, J = 5.6 Hz, 1H), 4.00~3.97 (m, 1H), 3.54 (t, J = 24 Hz, 1H), 3.43~3.34 (m, 3H), 2.34~2.30 (m, 1H), 2.23 (s, 3H), 1.97~1.78 (m, 3H). ¹⁹F NMR (400 MHz, CD₃OD): δ −195.37 (s, F). Chiral RT = 3.299 min (chiral method A); ee = 100 %. E234: LC-MS: 383.1 [M + H]⁺. ¹H NMR (400 MHz, CD₃OD): δ 7.70 (s, 1H), 4.75 (t, J = 6.4 Hz, 1H), 4.60~4.51 (m, 1H), 4.49~4.39 (m, 2H), 4.38~4.24 (m, 3H), 4.17 (q, J = 8.0 Hz, 1H), 4.08~4.03 (m, 1H), |
| | 3.41 (t, J = 8.0 Hz, 1H), 2.40~2.35 (m, 2H), 2.23 (s, 3H), 1.85~1.82 |
| | (m, 2H). ¹⁹F NMR (400 MHz, CD₃OD): δ −232.21 (s, F). Chiral RT = |
| | 3.597 min (chiral method A); ee = 98.3%. |
| | E235: LC-MS: 383.1 [M + H]⁺. ¹H NMR (400 MHz, CD₃OD): δ 7.70 |
| | (s, 1H), 4.75 (q, J = 6.4 Hz, 1H), 4.60~4.45 (m, 2H), 4.40~4.23 (m, |
| | 3H), 4.17 (q, J = 7.2 Hz, 1H), 4.05 (q, J = 7.2 Hz, 1H), 3.41 (t, J = 8.0 |
| | Hz, 1H), 2.40~2.33 (m, 2H), 2.23 (s, 3H), 1.85~1.82 (m, 2H). ¹⁹F |
| | NMR (400 MHz, CD₃OD): δ −232.21 (s, F). Chiral RT = 4.460 min |
| | (chiral method A); ee = 98.1 %. |
| | E236: LC-MS: 383.1 [M + H]⁺. ¹H NMR (400 MHz, CD₃OD): δ 7.70 |
| | (s, 1H), 4.86~4.68 (m, 1H), 4.38~4.29 (m, 3H), 4.17 (q, J = 5.6 Hz, |
| | 1H), 3.99 (d, J = 8.0 Hz, 1H), 3.54 (t, J = 24 Hz, 1H), 3.43~3.37 (m, |
| | 3H), 2.36~2.27 (m, 1H), 2.23 (s, 3H), 1.97~1.78 (m, 3H). ¹⁹F NMR |
| | (400 MHz, CD₃OD): δ −195.35 (s, F). Chiral RT = 5.352 min (chiral |
| | method A); ee = 99.5 %. |
| 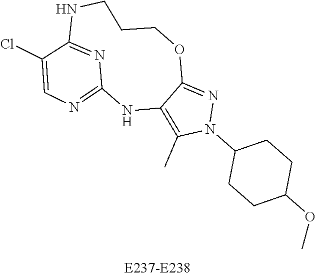 [SMILES: COC1CCC(n2nc3c(c2C)Nc2ncc(Cl)c(n2)NCCCO3)CC1] | Isomers 1-2: 14-Chloro-5-(4-methoxycyclohexyl)-4-methyl-8- oxa-2,5,6,12,16,17-hexaazatricyclo[11.3.1.0^{3,7}]heptadeca-1(16), 3,6,13(17),14-pentaene (E237-E238) Compounds were prepared using procedures similar to those used for preparing E12, starting from D33 and 4-methoxycyclohexyl methanesulfonate. E237: LC-MS: 393.2 [M + H]⁺. ¹H NMR (400 MHz, CDCl₃): δ 7.81 (s, 1H), 6.15 (s, 1H), 5.54 (t, J = 5.8 Hz, 1H), 4.38 (t, J = 4.4 Hz, 2H), 3.89~3.83 (m, 1H), 3.51 (dd, J = 10.8, 6.0 Hz, 2H), 3.37 (s, 3H), 3.27~3.22 (m, 1H), 2.22~2.20 (m, 2H), 2.19 (s, 3H), 2.06~1.90 (m, 6H), 1.39~1.32 (m, 2H). Chiral RT = 3.411 min (chiral method A). E238: LC-MS: 393.2 [M + H]⁺. ¹H NMR (400 MHz, CDCl₃): δ 7.81 (s, 1H), 6.08 (s, 1H), 5.52 (s, 1H), 4.40 (t, J = 4.8 Hz, 2H), 3.96~3.81 (m, 1H), 3.53~3.47 (m, 3H), 3.34 (s, 3H), 2.29~2.17 (m, 2H), 2.19 (s, 3H), 2.11 (d, J = 14.4 Hz, 2H), 1.92~1.88 (m, 2H), 1.68~1.64 (m, 2H), 1.49 (t, J = 13.4 Hz, 2H). Chiral RT = 7.270 min (chiral method A). |
| 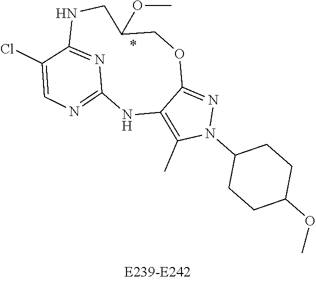 [SMILES: COC1CCC(n2nc3c(c2C)Nc2ncc(Cl)c(n2)NCC(OC)CO3)CC1] | Enantiomers 1-4: 14-Chloro-10-methoxy-5-(4-methoxycyclo hexyl)-4-methyl-8-oxa-2,5,6,12,16,17-hexaazatricyclo [11.3.1. 0^{3,7}]heptadeca-1(16),3,6,13(17),14-pentaene (E239-E242) Compounds were prepared using procedures similar to those used for preparing E12, using D13 and 4-methoxycyclohexyl methanesulfonate as starting materials. E239: LC-MS: 423.3 [M + H]⁺. ¹H NMR (400 MHz, CDCl₃): δ 7.77 (s, 1H), 7.27 (br, 1H), 5.98 (br, 1H), 4.50 (d, J = 12.0 Hz, 1H), 4.29 (t, J = 8.0 Hz, 1H), 3.94~3.88 (m, 2H), 3.47~3.45 (m, 1H), 3.41 (s, 3H), 3.37~3.36 (m, 2H), 3.33 (s, 3H), 2.25-2.18 (m, 5H), 2.15-2.10 (m, 2H), 1.67~1.63 (m, 2H), 1.52~1.45 (m, 2H). Chiral RT = 4.558 min (chiral method A); ee = 100 %. E240: LC-MS: 423.2 [M + H]⁺. ¹H NMR (400 MHz, CDCl₃): δ 7.78 (s, 1H), 7.31 (br, 1H), 5.99 (br, 1H), 4.48 (d, J = 12.0 Hz, 1H), 4.26 (t, J = 8.0 Hz, 1H), 3.91~3.86 (m, 2H), 3.37 (s, 3H), 3.34~3.23 (m, 5H), 3.26~3.23 (m, 1H), 2.22~2.19 (m, 2H), 2.15-2.10 (m, 5H), 1.91- 1.90 (m, 2H), 1.38~1.29 (m, 2H). Chiral RT = 4.867 min (chiral method A); ee = 99.1 %. |
| | E241: LC-MS: 423.2 [M + H]⁺. ¹H NMR (400 MHz, CDCl₃): δ 7.79 (s, |
| | 1H), 7.02 (br, 1H), 5.90 (br, 1H), 4.48 (d, J = 12.0 Hz, 1H), 4.27 (t, |
| | J = 12.0 Hz, 1H), 3.94~3.83 (m, 2H), 3.37 (s, 3H), 3.36~3.31 (m, |
| | 5H), 3.28~3.22 (m, 1H), 2.21 (br, 6H), 2.14~1.90 (m, 3H), 1.38~1.29 |
| | (m, 2H). Chiral RT = 5.268 min (chiral method A); ee = 100 %. |
| | E242: LC-MS: 423.2 [M + H]⁺. ¹H NMR (400 MHz, CDCl₃): δ 7.82 (s, |
| | 1H), 6.80 (br, 1H), 5.75 (br, 1H), 4.49 (d, J = 12.0 Hz, 1H), 4.31 (t, |
| | J = 8.0 Hz, 1H), 3.93~3.86 (m, 2H), 3.47~3.42 (m, 1H), 3.41 (s, 3H), |
| | 3.34~3.23 (m, 5H), 2.26~2.22 (m, 2H), 2.19 (s, 3H), 2.16~2.10 (m, |
| | 2H), 1.68~1.65 (m, 2H), 1.52~1.45 (m, 2H). Chiral RT = 5.481 min |
| | (chiral method A); ee = 95.5%. |
| 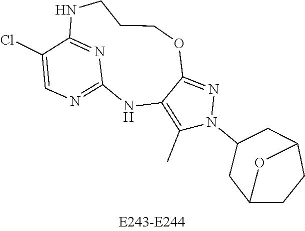 [SMILES: Cc1c2c(nn1C1CC3CCC(C1)O3)OCCCNc1nc(ncc1Cl)N2] | Isomers 1-2: 14-Chloro-4-methyl-5-{8-oxabicyclo[3.2.1] octan-3- yl}-8-oxa-2,5,6,12,16,17-hexaazatricyclo[11.3.1.0^{3,7}] heptadeca-1(16),3,6,13(17),14-pentaene (E243-E244) Compounds were prepared using procedures similar to those used for preparing E12, starting from D33 and 8-oxabicyclo[3.2.1]octan- 3-yl methanesulfonate. E243: LC-MS: 391.2 [M + H]⁺. ¹H NMR (400 MHz, CDCl₃): δ 7.81 (s, 1H), 6.30 (br, 1H), 5.57 (s, 1H), 4.53 (br, 2H), 4.38 (t, J = 4.0 Hz, 2H), 4.33~4.27 (m, 1H), 3.53~3.49 (m, 2H), 2.37 (t, J = 11.2 Hz, 2H), 2.21 (s, 3H), 2.05 (br, 2H), 1.91 (br, 2H), 1.81 (d, J = 7.2 Hz, 2H), 1.72~1.63 (m, 2H). E244: LC-MS: 391.2 [M + H]⁺. ¹H NMR (400 MHz, DMSO-d₆): δ 8.30 (s, 1H), 7.76 (s, 1H), 7.32 (s, 1H), 4.35 (br, 2H), 4.27 (t, J = 7.2 Hz, 1H), 4.20 (br, 2H), 3.28 (t, J = 5.2 Hz, 2H), 2.32~2.25 (m, 2H), 2.10 (s, 3H), 1.92 (d, J = 6.4 Hz, 2H), 1.87~1.72 (m, 6H). |
| 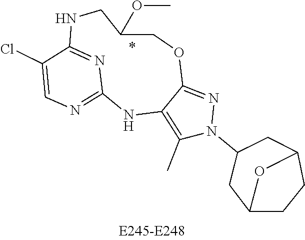 [SMILES: COC1CNc2nc(ncc2Cl)Nc2c(nn(C3CC4CCC(C3)O4)c2C)OC1] | Enantiomers 1-4: 14-Chloro-10-methoxy-4-methyl-5-{8-oxabi cyclo[3.2.1]octan-3-yl}-8-oxa-2,5,6,12,16,17-hexaazatricyclo [11.3.1.0^{3,7}]heptadeca-1(16),3,6,13(17),14-pentaene (E245-E248) Compounds were prepared using procedures similar to those used for preparing E12, using D13 and 8-oxabicyclo[3.2.1]octan-3-yl methanesulfonate as starting materials. E245: LC-MS: 421.1 [M + H]⁺. ¹H NMR (400 MHz, CDCl₃): δ 7.83 (s, 1H), 6.46 (s, 1H), 5.68 (br, 1H), 4.52~4.46 (m, 3H), 4.30 (t, J = 8.0 Hz, 2H), 3.89 (dd, J = 8.0 Hz, 1H), 3.42 (s, 3H), 3.34~3.26 (m, 2H), 2.41~2.35 (m, 2H), 2.16 (s, 3H), 2.04~1.86 (m, 6H). Chiral RT = 1.355 min (chiral method A); ee = 100%. E246: LC-MS: 421.1 [M + H]⁺. ¹H NMR (400 MHz, CDCl₃): δ 7.83 (s, 1H), 6.10 (s, 1H), 5.68 (br, 1H), 4.52~4.46 (m, 3H), 4.30 (t, J = 8.0 Hz, 2H), 3.89 (dd, J = 8.0 Hz, 1H), 3.41 (s, 3H), 3.34~3.26 (m, 2H), 2.37 (q, J = 12.0 Hz, 2H), 2.19 (s, 3H), 2.06~2.04 (m, 2H), 1.80 (d, J = 8.0 Hz, 2H), 1.71~1.68 (m, 2H). Chiral RT = 1.496 min (chiral |
| | method A); ee = 92.0%. |
| | E247: LC-MS: 421.2 [M + H]⁺. ¹H NMR (400 MHz, CDCl₃): δ 7.84 (s, |
| | 1H), 6.13 (s, 1H), 5.68 (br, 1H), 4.50~4.47 (m, 3H), 4.29 (t, J = 8 |
| | Hz, 2H), 3.89 (m, 1H), 3.42 (s, 3H), 3.36~3.28 (m, 2H), 2.41~2.36 |
| | (m, 2H), 2.16 (s, 3H), 2.04~1.86 (m, 6H), Chiral RT = 1.853 min |
| | (chiral method A); ee = 100%. |
| | E248: LC-MS: 421.2 [M + H]⁺. ¹H NMR (400 MHz, CDCl₃): δ 7.83 (s, |
| | 1H), 6.12 (s, 1H), 5.68 (br, 1H), 4.52~4.46 (m, 3H), 4.30 (t, J = 8.0 |
| | Hz, 2H), 3.89 (dd, J = 8.0 Hz, 1H), 3.41 (s, 3H), 3.34~3.26 (m, 2H), |
| | 2.38 (q, J = 12.0 Hz, 2H), 2.19 (s, 3H), 2.06~2.04 (m, 2H), 1.80 (d, |
| | J = 8.0 Hz, 2H), 1.71~1.66 (m, 2H). Chiral RT = 2.528 min (chiral |
| | method A); ee = 100%. |
| 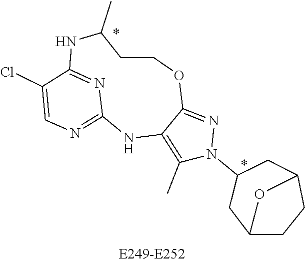 [SMILES: Cc1c2c(nn1C1CC3CCC(C1)O3)OCCC(C)Nc1nc(ncc1Cl)N2] | Enantiomers 1-4: 14-Chloro-4,11-dimethyl-5-{8- oxabicyclo[3.2.1] octan-3-yl}-8-oxa-2,5,6,12,16,17- hexaazatricyclo[11.3.1.0^{3,7}] heptadeca-1(16),3,6,13(17),14- pentaene (E249-E252) Compounds were prepared using procedures similar to those used for preparing E12, starting from D73 and 8-oxabicyclo[3.2.1]octan- 3-yl methanesulfonate. E249: LC-MS: 405.2 [M + H]⁺. ¹H NMR (400 MHz, CDCl₃): δ 7.74 (s, 1H), 7.37-7.36 (m, 1H), 5.43-5.41 (m, 1H), 4.49~4.46 (m, 3H), 4.31~ 4.25 (m, 2H), 4.06~4.03 (m, 1H), 2.42~2.35 (m, 2H), 2.20 (s, 3H), 1.98~1.95 (m, 5H), 1.92~1.80 (m, 3H), 1.35~1.34 (d, J = 6.8 Hz, 3H). Chiral RT = 3.794 min (chiral method A); ee = 100%. E250: LC-MS: 405.2 [M + H]⁺. ¹H NMR (400 MHz, CDCl₃): δ 7.74 (s, 1H), 6.97 (br, 1H), 5.33~5.32 (m, 1H), 4.48~4.46 (m, 3H), 4.32~ 4.26 (m, 2H), 4.05~4.02 (m, 1H), 2.44~2.33 (m, 2H), 2.18 (s, 3H), 2.00~1.90 (m, 5H), 1.88~1.80 (m, 3H), 1.34~1.28(d, J = 6.8 Hz, 3H). Chiral RT = 6.114 min (chiral method A); ee = 100%. |
| | E251: LC-MS: 405.2 [M + H]⁺. ¹H NMR (400 MHz, CDCl₃): δ 7.77 (s, |
| | 1H), 6.81(br, 1H), 5.29-5.27 (m, 1H), 4.52~4.44 (m, 3H), 4.34~4.28 |
| | (m, 2H), 4.03~4.01 (m, 1H), 2.40~2.32 (m, 3H), 2.21 (s, 3H), 2.07~ |
| | 2.04 (m, 2H), 1.89~1.80 (m, 4H), 1.72~1.66 (m, 2H),1.33~1.32 (d, |
| | J = 6.8 Hz, 3H). Chiral RT = 7.136 min (chiral method A); |
| | ee = 100%. |
| | E252: LC-MS: 405.2 [M + H]⁺. ¹H NMR (400 MHz, CDCl₃): δ 7.78 (s, |
| | 1H), 6.68(br, 1H), 5.24-5.22 (m, 1H), 4.52~4.44 (m, 3H), 4.34- |
| | 4.28(m, 2H), 4.02~4.01 (m, 1H), 2.41~2.33 (m, 3H), 2.20 (s, 3H), |
| | 2.06~2.04 (m, 2H), 1.88~1.78 (m, 4H), 1.73~1.68 (m, 2H), 1.33- |
| | 1.32(d, J = 6.8 Hz, 3H). Chiral RT = 8.025 min (chiral method A); |
| | ee = 100%. |
| 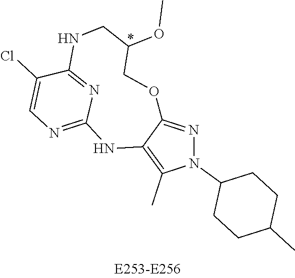 [SMILES: COC1CNc2nc(ncc2Cl)Nc2c(nn(C3CCC(C)CC3)c2C)OC1] | Enantiomers 1-4: 14-Chloro-10-methoxy-4-methyl-5-(4-methyl cyclohexyl)-8-oxa-2,5,6,12,16,17-hexaazatricyclo [11.3.1.0^{3,7}] heptadeca-1(16),3,6,13(17),14-pentaene (E253-E256) Compounds were prepared using procedures similar to those used for preparing E12, using D13 and 4-methylcyclohexyl methanesulfonate as starting materials. E253: LC-MS: 407.2 [M + H]⁺. ¹H NMR (400 MHz, CDCl₃): δ 7.86 (s, 1H), 6.09 (s, 1H), 5.70 (t, J = 11.2 Hz, 1H), 4.55 (d, J = 1.6 Hz, 1H), 4.44~4.40 (m, 1H), 4.15 (dd, J = 9.2, 2.4 Hz, 1H), 3.87 (br, 1H), 3.44 (s, 3H), 3.42~3.29 (m, 2H), 2.20 (s, 3H), 2.07~1.84 (m, 2H), 1.64~ 1.51 (m, 4H), 1.50~1.48 (m, 1H), 1.48~1.43 (m, 2H), 1.31 (d, J = 8.0 Hz, 3H). Chiral RT = 4.239 min (chiral method A); ee = 100%. E254: LC-MS: 407.2 [M + H]⁺. ¹H NMR (400 MHz, CDCl₃): δ 7.86 (s, 1H), 6.12 (s, 1H), 5.70 (t, J = 12.0 Hz, 1H), 4.54 (d, J = 11.2 Hz, 1H), 4.45~4.41 (m, 1H), 4.34~3.85 (m, 2H), 3.45 (s, 3H), 3.40~3.29 (m, 2H), 2.20 (s, 3H), 2.16~2.04 (s, 2H), 1.94~1.92 (m, 2H), 1.71~ 1.64(m, 5H), 1.58 (br, 1H), 1.30 (d, J = 8.0 Hz, 3H). Chiral RT = 4.639 min (chiral method A); ee = 100%. |
| | E255: LC-MS: 407.2 [M + H]⁺. ¹H NMR (400 MHz, CDCl₃): δ 7.86 (s, |
| | 1H), 6.11 (s, 1H), 5.70 (t, J = 12.8 Hz, 1H), 4.55 (d, J = 1.2 Hz, 1H), |
| | 4.45~4.40 (m, 1H), 4.16 (dd, J = 2.4, 14.0 Hz, 1H), 3.94~3.79 (m, |
| | 1H), 3.44 (s, 3H), 3.39~3.29 (s, 2H), 2.20 (s, 3H), 2.04~2.00 (m, |
| | 2H), 1.97~1.84 (m, 4H), 1.51 (br, 1H), 1.49~1.43 (m, 2H), 1.30 (d, |
| | J = 8.0 Hz, 3H). Chiral RT = 4.730 min (chiral method A); ee = 100%. |
| | E256: LC-MS: 407.2 [M + H]⁺. ¹H NMR (400 MHz, CDCl₃): δ 7.86 (s, |
| | 1H), 6.12 (s, 1H), 5.70 (t, J = 11.6 Hz, 1H), 4.54 (d, J = 11.2 Hz, 1H), |
| | 4.45~4.41 (m, 1H), 4.32~3.86 (m, 2H), 3.44 (s, 3H), 3.40~3.29 (m, |
| | 2H), 2.20 (s, 3H), 2.17~2.03 (m, 2H), 1.94~1.92 (m, 1H), 1.90~1.58 |
| | (m, 6H), 1.31 (d, J = 8.0 Hz, 3H). Chiral RT = 4.967 min (chiral |
| | method A); ee = 94.9%. |
| 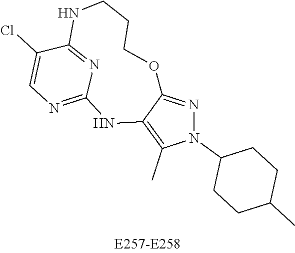 [SMILES: Cc1c2c(nn1C1CCC(C)CC1)OCCCNc1nc(ncc1Cl)N2] | Isomers 1-2: 14-Chloro-4-methyl-5-(4-methylcyclohexyl)-8-oxa- 2,5,6,12,16,17-hexaazatricyclo[11.3.1.0^{3,7}]heptadeca-1(16),3,6,13 (17),14-pentaene (E257-E258) Compounds were prepared using procedures similar to those used for preparing E12, starting from D33 and 4-methylcyclohexyl methanesulfonate. E257: LCMS: 377.4 [M + H]⁺. ¹H NMR (400 MHz, CDCl₃): δ 7.80 (s, 1H), 6.11 (s, 1H), 5.52 (s, 1H), 4.39 (t, J = 4.4 Hz, 2H), 3.84~3.78 (m, 1H), 3.54~3.49 (m, 3H), 2.18 (s, 3H), 1.99~1.82 (m, 8H), 1.12~ 1.02 (m, 2H), 0.93 (d, J = 6.4 Hz, 3H). Chiral RT = 2.408 min (chiral method C); ee = 100%. E258: LCMS: 377.4 [M + H]⁺. ¹H NMR (400 MHz, CDCl₃): δ 7.81 (s, 1H), 6.06 (m, 1H), 5.12~5.10 (d, J = 8.0 Hz, 1H), 4.52~4.48 (m, 1H), 4.34~4.29 (m, 1H), 4.03~4.02 (m, 1H), 3.84~3.78 (m, 1H), 2.18 (s, 3H), 2.02~1.95 (m, 4H), 1.90~1.77 (m, 4H), 1.53~1.47 (m, 2H), 1.32~1.25 (m, 3H), 1.22~1.03 (m, 1H), 1.02~1.00 (m, 3H). Chiral |
| | RT = 2.625 min (chiral method C); ee = 99.9%. |
| 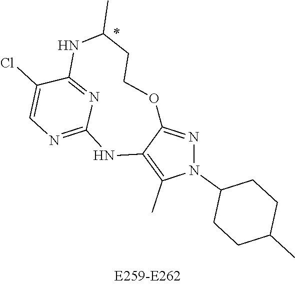 [SMILES: Cc1c2c(nn1C1CCC(C)CC1)OCCC(C)Nc1nc(ncc1Cl)N2] | Enantiomers 1-4: 14-Chloro-4,11-dimethyl-5-(4-methylcyclo hexyl)-8-oxa-2,5,6,12,16,17-hexaazatricyclo[11.3.1.0^{3,7}] heptadeca-1(16),3,6,13(17),14-pentaene (E259-E262) Compounds were prepared using procedures similar to those used for preparing E37, starting from D73 and 4-methylcyclohexyl methanesulfonate. E259: LC-MS: 391.2 [M + H]⁺. ¹H NMR (400 MHZ, CDCl₃): δ 7.81 (s, 1H), 6.06 (s, 1H), 5.11 (d, J = 8.0 Hz, 1H), 4.52~4.48 (m, 1H), 4.32 (t, J = 10.4 Hz, 1H), 4.03 (t, J = 3.6 Hz, 1H), 3.84~3.78 (m, 1H), 2.18 (s, 3H), 2.02~1.95 (m, 4H), 1.90~1.77 (m, 4H), 1.53~1.47 (m, 2H), 1.31 (d, J = 3.6 Hz, 3H), 1.25~1.06 (m, 1H), 1.01 (d, J = 8.0 Hz, 3H). Chiral RT = 5.939 min (chiral method C); ee = 100%. E260: LC-MS: 391.3 [M + H]⁺.¹H NMR (400 MHz, CDCl₃): δ 7.81 (s, 1H), 6.05 (s, 1H), 5.11 (d, J = 6.8 Hz, 1H), 4.52~4.48 (m, 1H), 4.34 (t, J = 10.4 Hz, 1H), 4.05~4.02 (m, 1H), 3.85 (t, J = 10.8 Hz, 1H), 2.18 (s, 3H), 2.16~2.00 (m, 4H), 1.93~1.78 (m, 4H), 1.69~1.62 (m, 2H), 1.30 (d, J = 6.8 Hz, 3H), 1.26~1.07 (m, 1H), 1.04 (d, J = 8.0 Hz, 3H). Chiral RT = 6.203 min (chiral method C); ee = 98.5%. |
| | E261: LC-MS: 391.3 [M + H]⁺. ¹H NMR (400 MHz, CDCl₃): δ 7.81 (s, |
| | 1H), 6.10 (s, 1H), 5.11 (d, J = 6.8 Hz, 1H), 4.52~4.48 (m, 1H), 4.34 |
| | (t, J = 10.4 Hz, 1H), 4.06~4.01 (m, 1H), 3.87~3.82 (m, 1H), 2.18 (s, |
| | 3H), 2.16~2.00 (m, 4H), 1.93~1.80 (m, 4H), 1.69~1.63 (m, 2H), 1.31 |
| | (d, J = 6.8 Hz, 3H), 1.25~1.07 (m, 1H), 1.03 (d, J = 8.0 Hz, 3H). |
| | Chiral RT = 6.748 min (chiral method C); ee = 100%. |
| | E262: LC-MS: 391.3 [M + H]⁺.¹H NMR (400 MHz, CDCl₃): δ 7.81 (s, |
| | 1H), 6.18 (s, 1H), 5.13 (d, J = 6.8 Hz, 1H), 4.52~4.48 (m, 1H), 4.32 |
| | (t, J = 10.2 Hz, 1H), 4.04~4.00 (m, 1H), 3.84~3.78 (m, 1H), 2.18 (s, |
| | 3H), 2.05~1.95 (m, 4H), 1.90~1.78 (m, 4H), 1.63~1.59 (m, 2H), 1.28 |
| | (d, J = 6.8 Hz, 3H), 1.26~1.07 (m, 1H), 1.05 (d, J = 8.0 Hz , 3H). |
| | Chiral RT = 9.431 min (chiral method C); ee = 100%. |
| 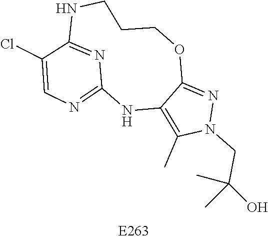 [SMILES: Cc1c2c(nn1CC(C)(C)O)OCCCNc1nc(ncc1Cl)N2] | 1-{14-Chloro-4-methyl-8-oxa-2,5,6,12,16,17-hexaazatricyclo [11.3.1.0^{3,7}]heptadeca-1(16),3,6,13(17),14-pentaen-5-yl}-2- methylpropan-2-ol (E263) Compound was prepared using procedures similar to those used for preparing E37, starting from D33 and 2,2-dimethyloxirane. LC-MS: 353.2 [M + H]⁺. ¹H NMR (400 MHz, CDCl₃): δ 7.82 (s, 1H), 6.17 (s, 1H), 5.56 (s, 1H), 4.63 (s, 1H), 4.37 (t, J = 4.6 Hz, 2H), 3.81 (s, 2H), 3.51 (dd, J = 10.8, 6.0 Hz, 2H), 2.19 (s, 3H), 1.95~1.91 (m, 2H), 1.16 (s, 6H). |
| 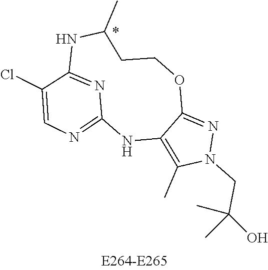 [SMILES: Cc1c2c(nn1CC(C)(C)O)OCCC(C)Nc1nc(ncc1Cl)N2] | Enantiomers 1-2: 1-{14-Chloro-4,11-dimethyl-8-oxa-2,5,6,12,16,17- hexaazatricyclo[11.3.1.0^{3,7}]heptadeca-1(17),3,6,13,15- pentaen-5-yl}-2-methylpropan-2-ol (E264-E265) Compounds were prepared using procedures similar to those used for preparing E37, starting from D73 and 2,2-dimethyloxirane. E264: LC-MS: 367.2 [M + H]⁺. ¹H NMR (400 MHz, CDCl₃): δ 7.80 (br, 1H), 7.08 (s, 1H), 5.36 (d, J = 6.8 Hz, 1H), 4.58 (br, 1H), 4.47~4.43 (m, 1H), 4.32 (t, J = 3.6 Hz, 1H), 4.02 (t, J = 3.6 Hz, 1H), 3.81 (s, 2H), 2.20 (s, 3H), 1.91~1.81 (m, 2H), 1.35 (d, J = 7.2 Hz, 3H), 1.21 (s, 3H), 1.11 (s, 3H). Chiral RT = 1.620 min (chiral method C); ee = 100%. E265: LC-MS: 367.2 [M + H]⁺. ¹H NMR (400 MHz, CDCl₃): δ 7.80 (s, 1H), 6.75 (s, 1H), 5.27 (d, J = 6.4 Hz, 1H), 4.61 (br, 1H), 4.46~4.43 (m, 1H), 4.33 (t, J = 11.2 Hz, 1H), 4.01 (t, J = 3.2 Hz, 1H), 3.81 (s, 2H), 2.20 (s, 3H), 1.94~1.79 (m, 2H), 1.33 (d, J = 7.2 Hz, 3H), 1.21 (s, 3H), 1.12 (s, 3H). Chiral RT = 1.832 min (chiral method C); ee = 100%. |
| 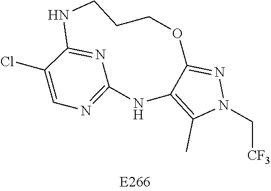 [SMILES: Cc1c2c(nn1CC(F)(F)F)OCCCNc1nc(ncc1Cl)N2] | 14-Chloro-4-methyl-5-(2,2,2-trifluoroethyl)-8-oxa-2,5,6,12,16,17- hexaazatricyclo[11.3.1.0^{3,7}]heptadeca-1(16),3,6,13(17),14- pentaene (E266) Compound was prepared using procedures similar to those used for preparing E12, starting from D33 and 1,1,1-trifluoro-2-iodoethane. LC-MS: 363.1 [M + H]⁺. ¹H NMR (400 MHz, CDCl₃): δ 7.82 (s, 1H), 6.23 (s, 1H), 5.57 (br, 1H), 4.46 (q, J = 8.4 Hz, 2H), 4.40 (t, J = 4.6 Hz, 2H), 3.52 (dd, J = 10.8, 6.4 Hz, 2H), 2.23 (s, 3H), 1.92 (t, J = 4.4 Hz, 2H). |
| 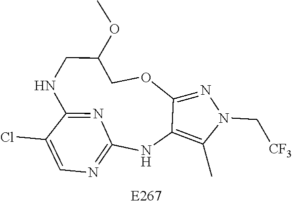 [SMILES: COC1CNc2nc(ncc2Cl)Nc2c(nn(CC(F)(F)F)c2C)OC1] | 14-Chloro-10-methoxy-4-methyl-5-(2,2,2-trifluoroethyl)-8-oxa- 2,5,6,12,16,17-hexaazatricyclo[11.3.1.0^{3,7}]heptadeca-1(16),3,6,13 (17),14-pentaene (E267) Compounds were prepared using procedures similar to those used for preparing E12, using D13 and 2,2,2-trifluoroethyl 4- methylbenzenesulfonate as starting materials. LC-MS: 393.1 [M + H]⁺. ¹H NMR (400 MHz, CDCl₃): δ 7.86 (s, 1H), 6.18 (s, 1H), 5.73 (s, 1H), 5.35 (s, 1H), 4.50~4.44 (m, 2H), 4.32 (t, J = 9.2 Hz, 1H), 3.94~3.89 (m, 1H), 3.42 (s, 3H), 2.23 (s, 3H), 2.02~2.00 (m, 2H). ¹⁹F NMR (400 MHz, CDCl₃): δ −70.90 (s, 1F). |
| 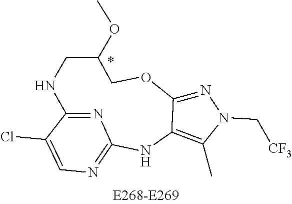 [SMILES: COC1CNc2nc(ncc2Cl)Nc2c(nn(CC(F)(F)F)c2C)OC1] | Enantiomers 1-2: 14-Chloro-10-methoxy-4-methyl-5-(2,2,2- trifluoroethyl)-8-oxa-2,5,6,12,16,17-hexaazatricyclo[11.3.1.0^{3,7}] heptadeca-1(16),3,6,13 (17),14-pentaene (E268-E269) Compounds were prepared from chiral separation of E267. E268: LC-MS: 393.2 [M + H]⁺. ¹H NMR (400 MHz, CDCl₃): δ 7.86 (s, 1H), 6.10 (s, 1H), 5.71 (br, 1H), 4.50~4.44 (m, 3H), 4.32 (t, J = 9.2 Hz, 1H), 3.94~3.89 (m, 1H), 3.42 (s, 3H), 3.31~3.26 (m, 2H), 2.22 (s, 3H). ¹⁹F NMR (376 MHz, CDCl₃): δ −70.90 (s, 3F). Chiral RT = 2.999 min (chiral method A); ee= 100%. E269: LC-MS: 393.2 [M + H]⁺. ¹H NMR (400 MHz, CDCl₃): δ 7.86 (s, 1H), 6.16 (s, 1H), 5.71 (br, 1H), 4.50~4.44 (m, 2H), 4.32 (t, J = 9.2 Hz, 1H), 3.94~3.89 (m, 1H), 3.42 (s, 3H), 3.32~3.27 (m, 2H), 2.22 (s, 3H). ¹⁹F NMR (376 MHz, CDCl₃): δ −70.90 (s, 3F). Chiral RT = |
| | 3.314 min (chiral method A); ee= 100%. |
| 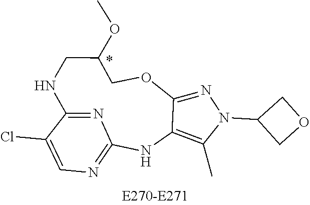 [SMILES: COC1CNc2nc(ncc2Cl)Nc2c(nn(C3COC3)c2C)OC1] | Enantiomers 1-2: 14-Chloro-10-methoxy-4-methyl-5-(oxetan-3- yl)-8-oxa-2,5,6,12,16,17-hexaazatricyclo[11.3.1.0^{3,7}]heptadeca- 1(16),3,6,13(17),14-pentaene (E270-E271) Compounds were prepared using procedures similar to those used for preparing E12, using D13 and 3-bromooxetane as starting materials. E270: LC-MS: 367.1 [M + H]⁺. ¹H NMR (400 MHz, CDCl₃): δ 7.87 (s, 1H), 6.28 (s, 1H), 5.73~5.71 (m, 1H), 5.30~5.26 (m, 1H), 5.25~5.20 (m, 1H), 5.17~5.14 (m, 1H), 4.92~4.90 (m, 2H), 4.55~4.51 (m, 1H), 4.44~4.41 (m, 1H), 3.95~3.89 (m, 1H), 3.49 (s, 3H), 3.34~3.28 (m, 3H), 2.15 (s, 3H). Chiral RT = 1.120 min (chiral method A); ee = 100%. E271: LC-MS: 367.1 [M + H]⁺. ¹H NMR (400 MHz, CDCl₃): δ 7.85 (s, |
| | 1H), 6.14 (s, 1H), 5.72~5.70 (m, 1H), 5.30-5.26 (m, 1H), 5.25~5.20 |
| | (m, 1H), 5.17-5.14 (m, 1H), 4.92~4.89 (m, 2H), 4.55~4.52(m, 1H), |
| | 4.43~4.38 (m, 1H), 3.95~3.89 (m, 1H), 3.43(s, 3H), 3.35~3.27 (m, |
| | 3H), 2.15(s, 3H). Chiral RT = 1.515 min (chiral method A); ee = |
| | 99.6%. |
| 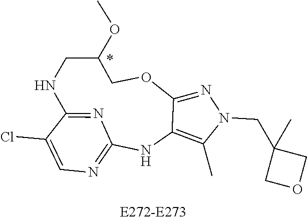 [SMILES: COC1CNc2nc(ncc2Cl)Nc2c(nn(CC3(C)COC3)c2C)OC1] | Enantiomers 1-2: 14-Chloro-10-methoxy-4-methyl-5-[(3-methyl oxetan-3-yl)methyl]-8-oxa-2,5,6,12,16,17-hexaazatricyclo [11.3.1.0^{3,7}]heptadeca-1(16),3,6,13(17),14-pentaene (E272-E273) Compounds were prepared using procedures similar to those used for preparing E12, using D13 and (3-methyloxetan-3-yl)methyl methanesulfonate as starting materials. E272: LC-MS: 395.2 [M + H]⁺. ¹H NMR (400 MHz, CDCl₃): δ 7.82 (s, 1H), 6.65 (s, 1H), 5.83~5.82 (m, 1H), 4.79~4.77 (m, 1H), 4.75~4.73 (m, 1H), 4.44~4.41 (m, 1H), 4.39~4.37 (m, 2H), 4.28~4.23 (m, 1H), 4.06~4.05 (m, 2H), 3.92~3.86 (m, 1H), 3.42 (s, 3H), 3.33~3.28 (m, 2H), 2.18 (s, 3H), 1.24 (s, 3H). Chiral RT = 3.986 min (chiral method A); ee = 100%. E273: LC-MS: 395.2 [M + H]⁺. ¹H NMR (400 MHz, CDCl₃): δ 7.82 (s, 1H), 6.66 (s, 1H), 5.83~5.81 (m, 1H), 4.79~4.78 (m, 1H), 4.75~4.73 |
| | (m, 1H), 4.44~4.42 (m, 1H), 4.39~4.37 (m, 2H), 4.28~4.23 (m, 1H), |
| | 4.06~4.05 (m, 2H), 3.92~3.86 (m, 1H), 3.42 (s, 3H), 3.33~3.28 (m, |
| | 2H), 2.18(s, 3H), 1.24(s, 3H). Chiral RT = 4.704 min (chiral method |
| | A); ee =96.3%. |
| 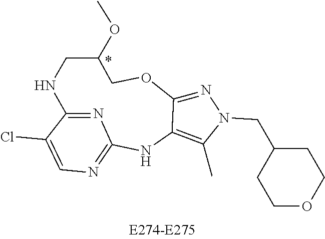 [SMILES: COC1CNc2nc(ncc2Cl)Nc2c(nn(CC3CCOCC3)c2C)OC1] | Enantiomers 1-2: 14-Chloro-10-methoxy-4-methyl-5-(oxan-4- ylmethyl)-8-oxa-2,5,6,12,16,17-hexaazatricyclo[11.3.1.0^{3,7}] heptadeca-1(16),3,6,13(17),14-pentaene (E274-E275) Compounds were prepared using procedures similar to those used for preparing E12, using D13 and (bromomethyl)cyclohexane as starting materials. E274: LC-MS: 409.3 [M + H]⁺. ¹H NMR (400 MHz, CDCl₃): δ 7.84 (s, 1H), 6.28 (s, 1H), 5.72~5.69 (m, 1H), 4.50~4.47 (m, 1H), 4.31~4.26 (m, 1H), 3.98~3.87 (m, 3H), 3.79~3.73 (m, 2H), 3.41 (s, 3H), 3.36~3.27 (m, 5H), 2.18~2.14 (m, 3H), 1.56~1.47 (m, 2H), 1.38~ 1.31 (m, 2H). Chiral RT = 1.104 min (chiral method A); ee = 100%. E275: LC-MS: 409.3 [M + H]⁺. ¹H NMR (400 MHz, CDCl₃): δ 7.84 (s, 1H), 6.24 (s, 1H), 5.72~5.69 (m, 1H), 4.50~4.47 (m, 1H), 4.31~4.26 (m, 1H), 3.98~3.87 (m, 3H), 3.79~3.73 (m, 2H), 3.41 (s, 3H), 3.36~3.27 (m, 5H), 2.18~2.14(m, 3H), 1.56~1.47 (m, 2H), 1.38~ 1.31(m, 2H). Chiral RT = 2.116 min (chiral method A); ee = 100%. |
| 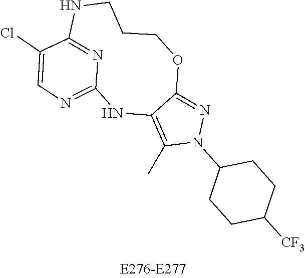 [SMILES: Cc1c2c(nn1C1CCC(C(F)(F)F)CC1)OCCCNc1nc(ncc1Cl)N2] | Isomers 1-2: 14-Chloro-4-methyl-5-[4-(trifluoromethyl)cyclo hexyl]-8-oxa-2,5,6,12,16,17-hexaazatricyclo[11.3.1.0^{3,7}] heptadeca-1(16),3,6,13(17),14-pentaene (E276-E277) Compounds were prepared using procedures similar to those used for preparing E12, starting from D33 and 4- (trifluoromethyl)cyclohexyl methanesulfonate. E276: LC-MS: 431.2 [M + H]⁺. ¹H NMR (400 MHz, CDCl₃): δ 7.80 (s, 1H), 6.28 (s, 1H), 5.55 (s, 1H), 4.38 (t, J = 4.6 Hz, 2H), 3.94~3.78 (m, 1H), 3.51 (dd, J = 10.8, 6.0 Hz, 2H), 2.19 (s, 3H), 2.15~2.09 (m, 3H), 2.01~1.97 (m, 4H), 1.93~1.90 (m, 2H), 1.55~1.43 (m, 2H). ¹⁹F NMR (376 MHz, CDCl₃): δ −73.57. E277: LC-MS: 431 .2 [M + H]⁺. ¹H NMR (400 MHz, CDCl₃): δ 7.79 (s, 1H), 6.81 (s, 1H), 5.70 (s, 1H), 4.37 (br, 2H), 4.13 (br, 1H), 3.53 (d, J = 4.8 Hz, 2H), 2.20 (s, 3H), 2.24~2.13 (m, 5H), 1.93~1.90 (br, 2H), 1.77~1.74 (m, 4H). ¹⁹F NMR (376 MHz, CDCl₃): δ −71.11. |
| 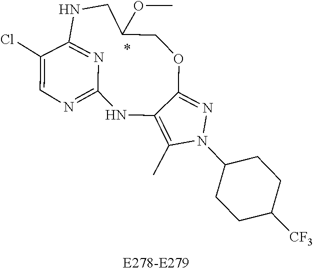 [SMILES: COC1CNc2nc(ncc2Cl)Nc2c(nn(C3CCC(C(F)(F)F)CC3)c2C)OC1] | Enantiomers 1-2: 14-Chloro-10-methoxy-4-methyl-5-[4-(trifluoro methyl)cyclohexyl]-8-oxa-2,5,6,12,16,17-hexaazatricyclo [11.3. 1.0^{3,7}]heptadeca-1(16),3,6,13(17),14-pentaene (E278-E279) Compounds were prepared using procedures similar to those used for preparing E12, using D13 and 4-(trifluoromethyl)cyclohexyl methanesulfonate as starting materials. E278: LC-MS: 461.2 [M + H]⁺. ¹H NMR (400 MHz, CDCl₃): δ 7.84 (s, 1H), 6.18 (s, 1H), 5.70~5.69 (m, 1H), 4.49~4.44 (m, 1H), 4.31~4.26 (m, 1H), 3.92~3.84 (m, 2H), 3.41 (s, 3H), 3.35~3.26 (m, 2H), 2.18 (s, 3H), 2.11~1.91(m, 7H), 1.53~1.46 (m, 2H). Chiral RT = 4.417 min (chiral method G); ee = 100%. E279: LC-MS: 461.2[M + H]⁺. ¹H NMR (400 MHz, CDCl₃): δ 7.84 (s, 1H), 7.26 (s, 1H), 5.88~5.69(m, 1H), 4.49~4.46 (m, 1H), 4.30~4.25 (m, 1H), 3.93~3.84 (m, 2H), 3.42 (s, 3H), 3.34~3.26 (m, 2H), 2.20 (s, 3H), 2.11~1.91(m, 7H), 1.53~1.46(m, 2H). Chiral RT = 5.518 min (chiral method G); ee = 100%. |
| 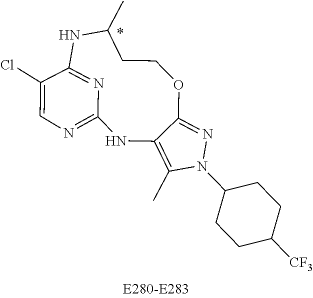 [SMILES: Cc1c2c(nn1C1CCC(C(F)(F)F)CC1)OCCC(C)Nc1nc(ncc1Cl)N2] | Enantiomers 1-4: 4-Chloro-4,11-dimethyl-5-[4-(trifluoromethyl) cyclohexyl]-8-oxa-2,5,6,12,16,17-hexaazatricyclo[11.3.1.0^{3,7}] heptadeca-1(16),3,6,13(17),14-pentaene (E280-E283) Compounds were prepared using procedures similar to those used for preparing E12, starting from D73 and 4- (trifluoromethyl)cyclohexyl methanesulfonate. E280: LC-MS: 445.2 [M + H]⁺. ¹H NMR (400 MHz, CDCl₃): δ 7.82 (s, 1H), 6.08 (s, 1H), 5.12 (d, J = 7.2 Hz, 1H), 4.50~4.46 (m, 1H), 4.31 (t, J = 10.8 Hz, 1H), 4.01 (t, J = 3.4 Hz, 1H), 3.87~3.84 (m, 1H), 2.19 (s, 3H), 2.13~2.04 (m, 4H), 1.96~1.93 (m, 2H), 1.90~1.83 (m, 2H), 1.80~1.78 (m, 1H), 1.50~1.31 (m, 2H), 1.29 (d, J = 8.0 Hz, 3H). ¹⁹F NMR (376 MHz, CDCl₃): δ −73.566 (s, 3F). Chiral RT = 1.859 min (chiral method A); ee = 100%. E281: LC-MS: 445.2 [M + H]⁺. ¹H NMR (400 MHz, CDCl₃): δ 7.82 (s, 1H), 6.11 (s, 1H), 5.12 (d, J = 6.8 Hz, 1H), 4.50~4.46 (m, 1H), 4.30 (t, J = 7.6 Hz, 1H), 4.04~3.99 (m, 1H), 3.89~3.82 (m, 1H), 2.35 (s, 3H), 2.13~2.04 (m, 4H), 1.96~1.93 (m, 2H), 1.91~1.83 (m, 2H), 1.81~1.78 (m, 1H), 1.51~1.33 (m, 2H), 1.31 (d, J = 8.0 Hz, 3H). ¹⁹F NMR (376 MHz, CDCl₃): δ −73.566 (s, 3F). Chiral RT = 2.792 min |
| | (chiral method A); ee = 100%. |
| | 282: LC-MS: 445.2 [M + H]⁺. ¹H NMR (400 MHz, CDCl₃): δ 7.82 (s, |
| | 1H), 6.03 (s, 1H), 5.11 (d, J = 6.8 Hz, 1H), 4.48~4.44 (m, 1H), 4.33 |
| | (t, J = 11.2 Hz, 1H), 4.12 (t, J = 4.8 Hz, 1H), 4.01 (t, J = 3.2 Hz, 1H), |
| | 2.34~2.25 (m, 2H), 2.18 (s, 3H), 2.15~2.04 (m, 2H), 1.94~1.87 (m, |
| | 2H), 1.83~1.72 (m, 5H), 1.28 (d, J = 8.0 Hz, 3H). ¹⁹F NMR (376 |
| | MHz, CDCl₃): δ −71.069 (s, 3F). Chiral RT= 3.590 min (chiral |
| | method A); ee = 100%. |
| | E283: LC-MS: 445.2[M + H]⁺. ¹H NMR (400 MHz, CDCl₃): δ 7.82 (s, |
| | 1H), 6.12 (s, 1H), 5.11 (d, J = 6.8 Hz, 1H), 4.48~4.44 (m, 1H), 4.34 |
| | (t, J = 10.8 Hz, 1H), 4.12 (t, J = 4.8 Hz, 1H), 4.03~3.98 (m, 2H), |
| | 2.34- 2.25 (m, 2H), 2.18 (s, 3H), 2.15-2.04 (m, 2H), 1.94~1.93 (m, |
| | 1H), 1.90~1.62 (m, 5H), 1.29 (d, J = 8.0 Hz, 3H). ¹⁹F NMR (376 |
| | MHz, CDCl₃): δ −71.065 (s, 3F). Chiral RT = 4.124 min (chiral |
| | method A); ee = 100%. |
| 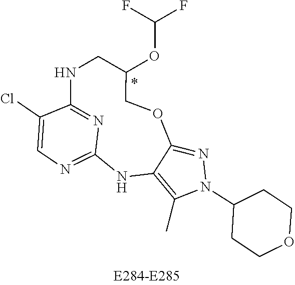 [SMILES: Cc1c2c(nn1C1CCOCC1)OCC(OC(F)F)CNc1nc(ncc1Cl)N2] | Enantiomers 1-2: 14-Chloro-10-(difluoromethoxy)-4-methyl-5- (oxan-4-yl)-8-oxa-2,5,6,12,16,17-hexaazatricyclo[11.3.1.0^{3,7}] heptadeca-1(16),3,6,13(17),14-pentaene (E284-E285) Compounds were prepared using procedures similar to those used for preparing E5, starting from 3-amino-2-(difluoromethoxy)propan- 1-ol. E284: LC-MS: 431.2 [M + H]⁺. ¹H NMR (400 MHz, CDCl₃): δ 7.87 (s, 1H), 6.28 (t, J = 72.0 Hz, 1H), 6.11 (s, 1H), 5.69 (t, J = 6.0 Hz, 1H), 4.54~4.43 (m, 2H), 4.28~4.25 (m, 1H), 4.12~4.00 (m, 3H), 3.54~ 3.39 (m, 3H), 2.34~2.21 (m, 2H), 2.21 (s, 3H), 1.78 (t, J = 13.2 Hz, 2H). ¹⁹F NMR (376 MHz, CDCl₃): δ −80.83 (d, J = 159.4 Hz, 1F), −82.38 (d, J = 160.6Hz, 1F). Chiral RT = 3.957 min (chiral method A); ee = 100%. E285: LC-MS: 431.2 [M + H]⁺. ¹H NMR (400 MHz, CDCl₃): δ 7.88 (s, 1H), 6.28 (t, J = 74.0 Hz, 1H), 6.09 (s, 1H), 5.69 (t, J = 6.0 Hz, 1H), 4.52~4.43 (m, 2H), 4.27~4.25 (m, 1H), 4.12~4.00 (m, 4H), 3.54~ 3.40 (m, 3H), 2.31~2.21 (m, 2H), 2.21 (s, 3H), 1.78 (t, J = 13.8 Hz, 2H). ¹⁹F NMR (376 MHz, CDCl₃): δ −80.82 (d, J = 159.4 Hz, |
| | 1F), −82.39 (d, J = 160.6 Hz, 1F). Chiral RT = 4.974 min (chiral |
| | method A); ee = 100%. |
| 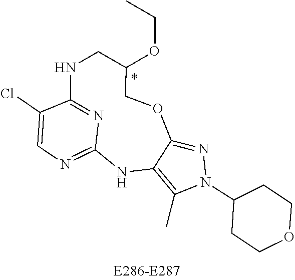 [SMILES: CCOC1CNc2nc(ncc2Cl)Nc2c(nn(C3CCOCC3)c2C)OC1] | Enantiomers 1-2: 14-Chloro-10-ethoxy-4-methyl-5-(oxan-4-yl)-8- oxa-2,5,6,12,16,17-hexaazatricyclo[11.3.1.0^{3,7}]heptadeca- 1(16),3,6,13(17),14-pentaene (E286-E287) Compounds were prepared using procedures similar to those used for preparing E2, starting from D10 and iodoethane. E286: LC-MS: 409.2 [M + H]⁺. ¹H NMR (400 MHz, CDCl₃): δ 7.84 (s, 1H), 6.07 (s, 1H), 5.68 (br, 1H), 4.47 (d, J = 10.4 Hz, 1H), 4.34 (t, J = 3.6 Hz, 1H), 4.12~4.06 (m, 3H), 3.94~3.89 (m, 1H), 3.64~3.42 (m, 6H), 3.33~2.28 (m, 1H), 2.32~2.21 (m, 1H), 2.17 (s, 3H), 1.78 (t, J = 13.6 Hz, 2H), 1.21 (t, J = 7.2 Hz, 3H). Chiral RT = 1.740 min (chiral method E); ee =100%. E287: LC-MS: 409.2 [M + H]⁺. ¹H NMR (400 MHz, CDCl₃): δ 7.84 (s, 1H), 6.10 (s, 1H), 5.69 (s, 1H), 4.47 (d, J = 10.4 Hz, 1H), 4.34 (t, J = 3.6 Hz, 1H), 4.12~4.07 (m, 3H), 3.94~3.90 (m, 1H), 3.62~3.43 (m, 6H), 3.41~2.28 (m, 1H), 2.32~2.21 (m, 1H), 2.17 (s, 3H), 1.78 (t, J = 13.6 Hz, 2H), 1.21 (t, J = 7.2 Hz, 3H). Chiral RT = 2.250 min (chiral method E); ee = 98.9%. |
| 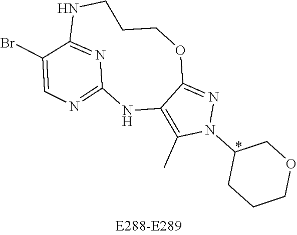 [SMILES: Cc1c2c(nn1C1CCCOC1)OCCCNc1nc(ncc1Br)N2] | Enantiomers 1-2: 14-Bromo-4-methyl-5-(oxan-3-yl)-8-oxa-2,5,6, 12,16,17-hexaazatricyclo[11.3.1.0^{3,7}]heptadeca-1(16),3,6,13(17), 14-pentaene (E288-E289) Compounds were prepared using procedures similar to those used for prparing E29, using D25 and tert-butyl (3- bromopropyl)carbamate as starting materials. E288: LC-MS: 411.1 [M + H]⁺. ¹H NMR (400 MHz, CD₃OD): δ 7.79 (s, 1H), 4.32~4.30 (m, 2H), 4.15~4.13 (m, 1H), 3.93~3.89 (m, 2H), 3.67~3.62 (m, 1H), 3.45~3.30 (m, 3H), 2.22 (s, 3H), 2.15~2.13 (m, 1H), 2.05~2.03 (m, 1H), 1.83~1.81 (m, 4H). Chiral RT = 3.780 min (chiral method A); ee = 100%. E289: LC-MS: 411.1 [M + H]⁺. ¹H NMR (400 MHz, CD₃OD): δ 7.79 (s, 1H), 4.32~4.30 (m, 2H), 4.16~4.11 (m, 1H), 3.93~3.89 (m, 2H), 3.67~3.62 (m, 1H), 3.45~3.31 (m, 3H), 2.22 (s, 3H), 2.19~2.12 (m, 1H), 2.05~2.02 (m, 1H), 1.83~1.81 (m, 4H). Chiral RT = 7.244 min (chiral method A); ee = 100%. |
| 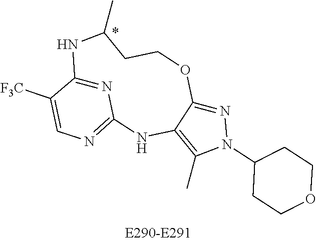 [SMILES: Cc1c2c(nn1C1CCOCC1)OCCC(C)Nc1nc(ncc1C(F)(F)F)N2] | Enantiomers 1-2: 4,11-Dimethyl-5-(oxan-4-yl)-14- (trifluoromethyl) -8-oxa-2,5,6,12,16,17- hexaazatricyclo[11.3.1.0^{3,7}]heptadeca-1(16),3,6,13(17),14- pentaene (E290-E291) Compounds were prepared using procedures similar to those used for preparing E31, using 3-((tert-butoxycarbonyl)amino)butyl methanesulfonate and 2,4-dichloro-5-(trifluoromethyl)pyrimidine as starting materials. E290: LC-MS: 413.2 [M + H]⁺. ¹H NMR (400 MHz, CD₃OD): δ 8.01 (s, 1H), 4.37~4.27 (m, 3H), 4.08~4.04 (m, 3H), 3.60~3.53 (m, 2H), 2.24~2.11 (m, 5H), 1.83~1.75 (m, 4H), 1.28~1.22 (m, 3H). ¹⁹F NMR (400 MHz, CD₃OD): δ −62.61 (s, 3F). Chiral RT = 1.533 min (chiral method B); ee = 100%. E291: LC-MS: 413.2 [M + H]⁺. ¹H NMR (400 MHz, CD₃OD): δ 8.01 (s, 1H), 4.38~4.27 (m, 3H), 4.08~4.03 (m, 3H), 3.60~3.54 (m, 2H), 2.24~ 2.20 (m, 5H), 1.83~1.74 (m, 4H), 1.30~1.26 (m, 3H). ¹⁹F NMR |
| | (400 MHz, CD₃OD): δ −62.57 (s, 3F). Chiral RT = 2.260 min (chiral |
| | method B); ee = 100%. |
| 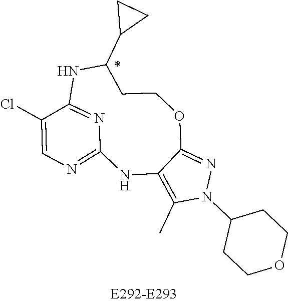 [SMILES: Cc1c2c(nn1C1CCOCC1)OCCC(C1CC1)Nc1nc(ncc1Cl)N2] | Enantiomers 1-2: 14-Chloro-11-cyclopropyl-4-methyl-5-(oxan-4- yl)-8-oxa-2,5,6,12,16,17-hexaazatricyclo[11.3.1.0^{3,7}]heptadeca- 1(16),3,6,13(17),14-pentaene (E292-E293) Compounds were prepared using procedures similar to those used for preparing E5, starting from 3-amino-3-cyclopropylpropan-1-ol. E292: LC-MS: 405.2 [M + H]⁺. ¹H NMR (400 MHz, CDCl₃): δ 7.81 (s, 1H), 6.08 (s, 1H), 5.42 (d, J = 6.0 Hz, 1H), 4.56 (dd, J = 11.6, 3.6 Hz, 1H), 4.25 (t, J = 10.8 Hz, 1H), 4.22~4.03 (m, 3H), 3.50 (t, J = 12.0 Hz, 2H), 3.16~3.12 (m, 1H), 2.30~2.22 (m, 2H), 2.19 (s, 3H), 2.15~2.09 (m, 1H), 1.95~1.91 (m, 1H), 1.78 (t, J = 13.2 Hz, 2H), 1.09~0.97 (m, 1H), 0.65~0.52 (m, 2H), 0.37~0.19 (m, 2H). Chiral RT = 2.404 min (chiral method B); ee = 100%. E293: LC-MS: 405.2 [M + H]⁺. ¹H NMR (400 MHz, CDCl₃): δ 7.81 (s, 1H), 6.13 (s, 1H), 5.43 (d, J = 6.4 Hz, 1H), 4.56 (dd, J = 11.6, 3.6 Hz, 1H), 4.25 (t, J = 10.8 Hz, 1H), 4.22~4.03 (m, 3H), 3.50 (t, J = 12.0 Hz, 2H), 3.16~3.12 (m, 1H), 2.30~2.22 (m, 2H), 2.19 (s, 3H), 2.15~2.09 (m, 1H), 1.95~1.91 (m, 1H), 1.78 (t, J = 14.4 Hz, 2H), 1.09~0.97 (m, 1H), 0.65~0.56 (m, 2H), 0.37~0.28 (m, 2H). Chiral RT = 3.172 min (chiral method B); ee = 99.9%. |
| 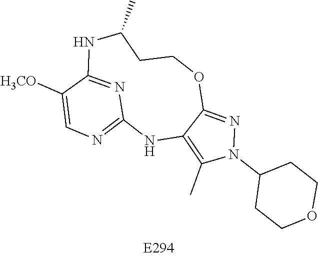 [SMILES: COc1cnc2nc1N[C@H](C)CCOc1nn(C3CCOCC3)c(C)c1N2] | (11R)-14-Methoxy-4,11-dimethyl-5-(oxan-4-yl)-8-oxa-2,5,6,12,16, 17-hexaazatricyclo[11.3.1.0^{3,7}]heptadeca-1(16),3,6,13(17),14- pentaene (E294) Compound was prepared using procedures similar to those used for preparing E31, using (R)-3-((tert-butoxycarbonyl)amino)butyl methanesulfonate and 2,4-dichloro-5-methoxypyrimidine as starting materials. LC-MS: 375.3 [M + H]⁺. ¹H NMR (400 MHz, CDCl₃): δ 8.02 (s, 1H), 5.64~5.62 (m, 1H), 4.51~4.30 (m, 1H), 4.28~4.25 (m, 1H), 4.11~3.96 (m, 4H), 3.78 (br, 3H), 3.67~3.60 (m, 1H), 3.54~3.48 (m, 2H), 2.26 (s, 3H), 1.89~1.43 (m, 4H), 1.35~1.31 (m, 3H), 1.30~1.28 (m, 2H). |
| 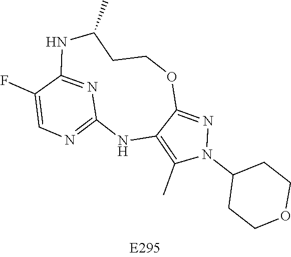 [SMILES: Cc1c2c(nn1C1CCOCC1)OCC[C@@H](C)Nc1nc(ncc1F)N2] | (11R)-14-Fluoro-4,11-dimethyl-5-(oxan-4-yl)-8-oxa- 2,5,6,12,16,17-hexaazatricyclo[11.3.1.0^{3,7}]heptadeca- 1(16),3,6,13(17),14-pentaene (E295) Compound was prepared using procedures similar to those used for preparing E31, using (R)-3-((tert-butoxycarbonyl)amino)butyl methanesulfonate and 2,4-dichloro-5-fluoropyrimidine as starting materials. LC-MS: 363.3 [M + H]⁺. ¹H NMR (400 MHz, CDCl₃): δ 7.68 (s, 1H), 6.62 (s, 1H), 5.03~5.02 (m, 1H), 4.51~4.89 (m, 1H), 4.35~4.30 (m, 1H), 4.09~4.04 (m, 4H), 3.53~3.48 (m, 2H), 2.31~2.28 (m, 4H), 2.22 (s, 3H), 1.96~1.75 (m, 2H), 1.34~1.32 (m, 3H). |
| 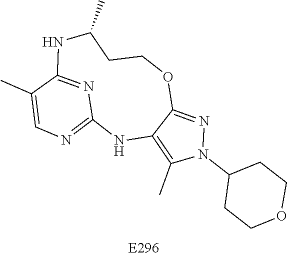 [SMILES: Cc1cnc2nc1N[C@H](C)CCOc1nn(C3CCOCC3)c(C)c1N2] | (11R)-14-Methyl-4,11-dimethyl-5-(oxan-4-yl)-8-oxa- 2,5,6,12,16,17 -hexaazatricyclo[11.3.1.0^{3,7}]heptadeca- 1(16),3,6,13(17),14-pentaene (E296) Compound was prepared using procedures similar to those used for preparing E31, using (R)-3-((tert-butoxycarbonyl)amino)butyl methanesulfonate and 2,4-dichloro-5-methylpyrimidine as starting materials. LC-MS: 359.4 [M + H]⁺. ¹H NMR (400 MHz, CDCl₃): δ 7.65 (s, 1H), 5.97 (s, 1H), 4.51~4.43 (m, 2H), 4.38~4.33 (m, 1H), 4.12~4.03 (m, 4H), 3.54~3.47 (m, 2H), 3.35~2.23 (m, 2H), 2.22 (s, 3H), 1.92 (s, 3H), 1.88~1.79 (m, 2H), 1.75~1.71 (m, 2H), 1.30~1.28 (m, 3H). |
| 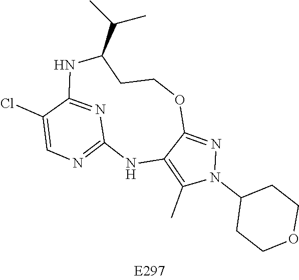 [SMILES: Cc1c2c(nn1C1CCOCC1)OCC[C@H](C(C)C)Nc1nc(ncc1Cl)N2] | (11R)-14-Chloro-4-methyl-5-(oxan-4-yl)-11-(propan-2-yl)-8-oxa- 2,5,6,12,16,17-hexaazatricyclo[11.3.1.0^{3,7}]heptadeca- 1(16),3,6,13(17),14-pentaene (E297) Compound was prepared using procedures similar to those used for preparing E5, starting from (R)-3-amino-4-methylpentan-1-ol. LC-MS: 407.3 [M + H]⁺. ¹H NMR (400 MHz, CDCl₃): δ 7.81 (s, 1H), 6.28 (s, 1H), 5.25 (d, J = 7.6 Hz, 1H), 4.53 (dd, J = 11.6, 3.6 Hz, 1H), 4.26 (t, J = 10.8 Hz, 1H), 4.12~4.07 (m, 3H), 3.72~3.70 (m, 1H), 3.54~3.48 (m, 2H), 2.31~2.21 (m, 2H), 2.21 (s, 3H), 1.98 (dt, J = 16.1, 4.6 Hz, 1H), 1.88~1.77 (m, 3H), 1.72~1.66 (m, 1H), 1.00 (d, J = 6.4 Hz, 3H), 0.99 (d, J = 6.4 Hz, 3H). |
| 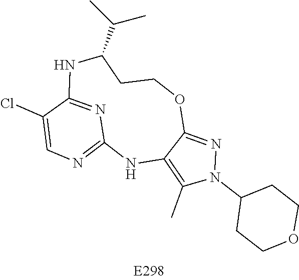 [SMILES: Cc1c2c(nn1C1CCOCC1)OCC[C@@H](C(C)C)Nc1nc(ncc1Cl)N2] | (11S)-14-Chloro-4-methyl-5-(oxan-4-yl)-11-(propan-2-yl)-8-oxa- 2,5,6,12,16,17-hexaazatricyclo[11.3.1.0^{3,7}]heptadeca- 1(16),3,6,13(17),14-pentaene (E298) Compound was prepared using procedures similar to those used for preparing E5, starting from (S)-3-amino-4-methylpentan-1-ol. LC-MS: 407.3 [M + H]⁺. ¹H NMR (400 MHz, CDCl₃): δ 7.82 (s, 1H), 6.39 (s, 1H), 5.25 (d, J = 7.6 Hz, 1H), 4.53 (dd, J = 11.6, 3.6 Hz, 1H), 4.28 (t, J = 10.8 Hz, 1H), 4.13~4.09 (m, 3H), 3.72~3.70 (m, 1H), 3.56~3.50 (m, 2H), 2.30~2.23 (m, 2H), 2.23 (s, 3H), 2.03~1.98 (m, 1H), 1.87~1.81 (m, 3H), 1.72~1.70 (m, 1H), 1.02 (d, J = 6.8 Hz, 3H), 1.01 (d, J = 6.8 Hz, 3H). |
| 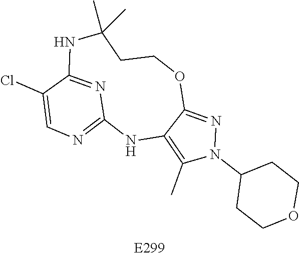 [SMILES: Cc1c2c(nn1C1CCOCC1)OCCC(C)(C)Nc1nc(ncc1Cl)N2] | 14-Chloro-4,11,11-trimethyl-5-(oxan-4-yl)-8-oxa-2,5,6,12,16,17- hexaazatricyclo[11.3.1.0^{3,7}]heptadeca-1(16),3,6,13(17),14- pentaene (E299) Compound was prepared using procedures similar to those used for preparing E1, starting from tert-butyl (4-hydroxy-2-methylbutan-2- yl)carbamate. LC-MS: 393.4 [M + H]⁺. ¹H NMR (400 MHz, CDCl₃): δ 7.77 (s, 1H), 6.20 (s, 1H), 5.14 (s, 1H), 4.68~4.38 (m, 2H), 4.07 (dd, J = 16.8, 11.3 Hz, 3H), 3.51 (t, J = 11.4 Hz, 2H), 2.25 (dd, J = 12.2, 3.8 Hz, 2H), 2.21 (s, 3H), 2.19~2.14 (m, 2H), 1.77 (d, J = 12.7 Hz, 2H), 1.36 (s, 6H). |
| 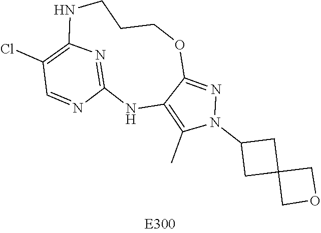 [SMILES: Cc1c2c(nn1C1CC3(COC3)C1)OCCCNc1nc(ncc1Cl)N2] | 14-Chloro-4-methyl-5-{2-oxaspiro[3.3]heptan-6-yl}-8-oxa- 2,5,6,12,16,17-hexaazatricyclo[11.3.1.0^{3,7}]heptadeca- 1(16),3,6,13(17),14-pentaene (E300) Compound was prepared using procedures similar to those used for Eirlo preparing E12, starting from D33 and 2-oxaspiro[3.3]heptan-6-yl methanesulfonate. LC-MS: 377.1 [M + H]⁺. ¹H NMR (400 MHz, CDCl₃): δ 7.80 (s, 1H), 6.27 (br, 1H), 5.58 (br, 1H), 4.79 (s, 2H), 4.73 (s, 2H), 4.48~4.27 (m, 3H), 3.63~3.39 (m, 2H), 2.90~2.76 (m, 2H), 2.72~2.57 (m, 2H), 2.14 (s, 3H), 1.93 (br, 2H). |
| 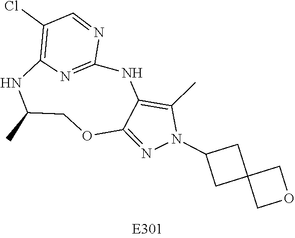 | (11R)-14-Chloro-4,11-dimethyl-5-{2-oxaspiro[3.3]heptan-6-yl}-8- oxa-2,5,6,12,16,17-hexaazatricyclo[11.3.1.0^{3,7}]heptadeca- 1(17),3,6,13,15-pentaene (E301) Compound was prepared using procedures similar to those used for preparing E46, starting from using D79 and 2-oxaspiro[3.3]heptan-6-yl methanesulfonate. LC-MS: 391.2 [M + H]⁺. ¹H NMR (400 MHz, CDCl₃): δ 7.70 (s, 1H), 5.68 (s, 1H), 4.79 (s, 2H), 4.73 (s, 2H), 4.52~4.26 (m, 3H), 4.15~ 4.00 (m, 1H), 2.92~2.82 (m, 1H), 2.79~2.57 (m, 3H), 2.20 (s, 3H), 1.94~1.87 (m, 2H), 1.38 (d, J = 7.2 Hz, 3H). |
| 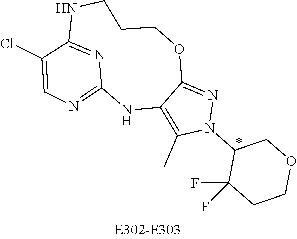 [SMILES: Cc1c2c(nn1C1COCCC1(F)F)OCCCNc1nc(ncc1Cl)N2] | Enantiomer 1-2: 14-Chloro-5-(4,4-difluorooxan-3-yl)-4-methyl-8- oxa-2,5,6,12,16,17-hexaazatricyclo[11.3.1.0^{3,7}]heptadeca- 1(16),3,6,13(17),14-pentaene (E302-E303) Compounds were prepared using procedures similar to those used for preparing E86, (directly reacting D118 with DAST before cyclization). E302: LC-MS: 401.2 [M + H]⁺. ¹H NMR (400 MHz, DMSO-d₆): δ 8.44 (s, 1H), 7.83 (s, 1H), 7.40 (t, J = 5.6 Hz, 1H), 4.70~4.63 (m, 1H), 4.24~4.04 (m, 5H), 3.68~3.62 (m, 1H), 3.35~3.33 (m, 2H), 2.30~2.25 (m, 2H), 2.21 (s, 3H),1.85~1.71 (m, 2H). Chiral RT = 3.353 min (chiral method C); ee = 100%. E303: LC-MS: 401.2 [M + H]⁺. ¹H NMR (400 MHz, DMSO-d₆): δ 8.44 (s, 1H), 7.83 (s, 1H), 7.41 (t, J = 6.0 Hz, 1H), 4.70~4.63 (m, 1H), 4.28 ~4.04 (m, 5H), 3.68~3.62 (m, 1H), 3.35~3.28 (m, 2H), 2.30~2.25 (m, 2H), 2.21 (s, 3H),1.85~1.71 (m, 2H). Chiral RT = 4.089 min (chiral method C); ee = 100%. |
| 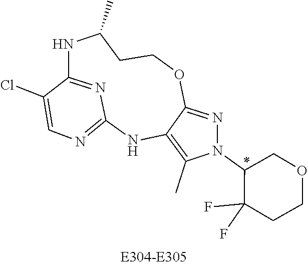 [SMILES: Cc1c2c(nn1C1COCCC1(F)F)OCC[C@@H](C)Nc1nc(ncc1Cl)N2] | Enantiomers 1-2: (11R)-14-Chloro-5-(4,4-difluorooxan-3-yl)- 4,11-dimethyl-8-oxa-2,5,6,12,16,17-hexaazatricyclo[11.3.1.0^{3,7}] heptadeca-1(16),3,6,13(17),14-pentaene (E304-E305) Compounds were prepared using procedures similar to those used for preparing E302. E304: LC-MS: 415.3 [M + H]⁺. ¹H NMR (400 MHz, CD₃OD): δ 7.72 (s, 1H), 4.53~4.45 (m, 1H), 4.40~4.36 (m, 2H), 4.31 (t, J = 24 Hz, 1H), 4.23~4.18 (m, 3H), 4.04~3.96 (m, 1H), 2.31~2.17 (m, 5H), 1.80~ 1.76 (m, 2H), 1.28 (d, J = 8.0 Hz, 3H). Chiral RT = 5.137 min (chiral method A); ee = 100%. E305: LC-MS: 415.3 [M + H]⁺. ¹H NMR (400 MHz, CD₃OD): δ 7.72(s, 1H), 4.92~4.82 (m, 1H), 4.57~4.22 (m, 3H), 4.14~4.09 (m, 1H), 4.04~ 4.00 (m, 1H), 3.93~3.89 (m, 1H), 3.74~3.71 (m, 1H), 2.25~2.17(m, 5H), 1.77~1.74 (m, 2H), 1.28 (d, J = 8.0 Hz, 3H). Chiral RT = 6.255 min (chiral method A); ee = 98.7%. |
| 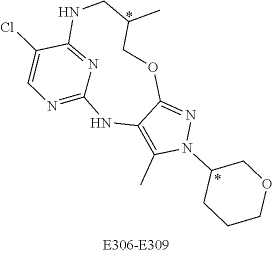 [SMILES: Cc1c2c(nn1C1CCCOC1)OCC(C)CNc1nc(ncc1Cl)N2] | Enantiomers 1-4: 14-Chloro-4,10-dimethyl-5-(oxan-3-yl)-8-oxa- 2,5,6,12,16,17-hexaazatricyclo[11.3.1.0^{3,7}]heptadeca-1(16),3,6, 13(17),14-pentaene (E306-E309) Compounds were prepared using procedures similar to those used for preparing E2, starting from D25 and 3-((tert- butoxycarbonyl)amino)-2-methylpropyl methanesulfonate. E306: LC-MS: 379.2 [M + H]⁺. ¹H NMR (400 MHz, CD₃OD): δ 7.70 (s, 1H), 4.20~4.10 (m, 3H), 3.93~3.91 (m, 2H), 3.70~3.59 (m, 2H), 3.45~.38 (m, 1H), 3.06 (d, J = 12.0 Hz, 1H), 2.22 (s, 3H), 2.16~2.11 (m, 1H), 2.04~1.99 (m, 1H), 1.81 (br, 3H), 0.99 (d, J = 8.0 Hz, 3H). Chiral RT = 1.856 min (chiral method A); ee = 99.1%. E307: LC-MS: 379.2 [M + H]⁺. ¹H NMR (400 MHz, CD₃OD): δ 7.70 (s, 1H), 4.22~4.08 (m, 3H), 3.93~3.86 (m, 2H), 3.66~3.59 (m, 2H), 3.45~3.38 (m, 1H), 3.03 (d, J = 16.0 Hz, 1H), 2.22 (s, 3H), 2.18~2.12 (m, 1H), 2.06~2.03 (m, 1H), 1.81 (br, 3H), 0.99 (d, J = 8.0 Hz, 3H). Chiral RT = 1.999 min (chiral method A); ee = 99.1%. |
| | E308: LC-MS: 379.2 [M + H]⁺. ¹H NMR (400 MHz, CD₃OD): δ 7.70 (s, |
| | 1H), 4.22~4.09 (m, 3H), 3.93~3.86 (m, 2H), 3.66~3.60 (m, 2H), |
| | 3.45~3.38 (m, 1H), 3.04 (d, J = 16.0 Hz, 1H), 2.22 (s, 3H), 2.18~2.13 |
| | (m, 1H), 2.06~2.01 (m, 1H), 1.81 (br, 3H), 0.99 (d, J = 8.0 Hz, 3H). |
| | Chiral RT = 2.359 min (chiral method A); ee = 100%. |
| | E309: LC-MS: 379.2 [M + H]⁺. ¹H NMR (400 MHz, CD₃OD): δ 7.70 (s |
| | 1H), 4.20~4.10 (m, 3H), 3.93~3.91 (m, 2H), 3.70~3.60 (m, 2H), |
| | 3.45~3.39 (m, 1H), 3.05 (d, J = 16.0 Hz, 1H), 2.22 (s, 3H), 2.16~2.08 |
| | (m, 1H), 2.03~2.01 (m, 1H), 1.81 (br, 3H), 0.99 (d, J = 8.0 Hz, 3H). |
| | Chiral RT = 3.418 min (chiral method A); ee = 100%. |
| 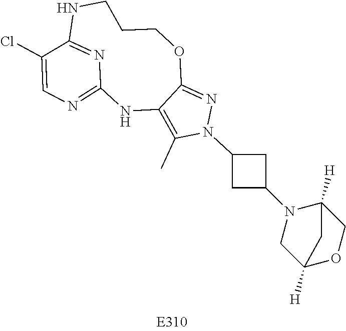 [SMILES: Cc1c2c(nn1C1CC(N3C[C@H]4C[C@@H]3CO4)C1)OCCCNc1nc(ncc1Cl)N2] | 14-Chloro-4-methyl-5-{3-[(1R,4R)-2-oxa-5-azabicyclo[2.2.1] heptan-5-yl]-cyclobutyl}-8-oxa-2,5,6,12,16,17-hexaazatricyclo [11.3.1.0^{3,7}]heptadeca-1(16),3,6,13(17),14-pentaene (E310) Compound was prepared using procedures similar to those used for preparing E45, starting from D75 and (1R,4R)-2-oxa-5- azabicyclo[2.2.1]heptane. LC-MS: 432.3 [M + H]⁺. ¹H NMR (400 MHz, DMSO-d₆): δ 8.30 (s, 1H), 7.76 (s, 1H), 7.32 (t, J = 5.6 Hz, 1H), 4.34 (br, 2H), 4.23 (d, J = 4.4 Hz, 2H), 3.77 (d, J = 6.8 Hz, 1H), 3.49 (d, J = 6.8 Hz, 1H), 3.43 (br, 1H), 3.30~3.27 (m, 2H), 3.00~2.97 (m, 1H), 2.72 (d, J = 9.6 Hz, 1H), 2.45~2.25 (m, 5H), 2.09 (s, 3H), 1.73~1.70 (m, 3H), 1.58~1.55 (m, 1H). |
| 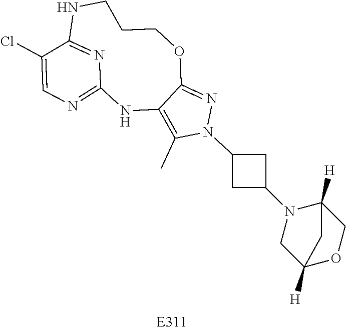 [SMILES: Cc1c2c(nn1C1CC(N3C[C@@H]4C[C@H]3CO4)C1)OCCCNc1nc(ncc1Cl)N2] | 14-Chloro-4-methyl-5-{3-[(1S,4S)-2-oxa-5-azabicyclo[2.2.1] heptan-5-yl]-cyclobutyl}-8-oxa-2,5,6,12,16,17-hexaazatricyclo [11.3.1.0^{3,7}]heptadeca-1(16),3,6,13(17),14-pentaene (E311) Compound was prepared using procedures similar to those used for preparing E45, starting from D75 and(1S,4S)-2-oxa-5- azabicyclo[2.2.1]heptanes. LC-MS: 432.3 [M + H]⁺. ¹H NMR (400 MHz, DMSO-d₆): δ 8.29 (s, 1H), 7.76 (s, 1H), 7.31 (s, 1H), 4.35 (br, 2H), 4.23 (br, 2H), 3.77 (d, J = 7.6 Hz, 1H), 3.49 (d, J = 6.8 Hz, 1H), 3.43 (br, 1H), 3.30~3.27 (m, 2H), 3.01~2.97 (m, 1H), 2.72 (d, J = 9.2 Hz, 1H), 2.45~2.25 (m, 5H), 2.09 (s, 3H), 1.73~1.68 (m, 3H), 1.57~1.55 (m, 1H). |
| 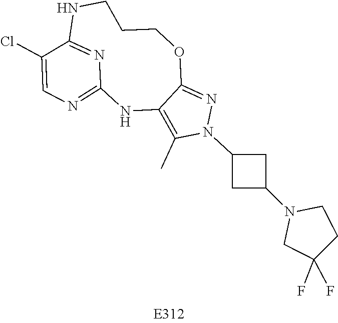 [SMILES: Cc1c2c(nn1C1CC(N3CCC(F)(F)C3)C1)OCCCNc1nc(ncc1Cl)N2] | 14-Chloro-5-[3-(3,3-difluoropyrrolidin-1-yl)cyclobutyl]-4-methyl- 8-oxa-2,5,6,12,16,17-hexaazatricyclo[11.3.1.0^{3,7}] heptadeca- 1(16),3,6,13(17),14-pentaene (E312) Compound was prepared using procedures similar to those used for preparing E45, starting from D75 with 3,3-difluoropyrrolidine. LC-MS: 440.2 [M + H]⁺. ¹H NMR (400 MHz, DMSO-d₆): δ 8.31 (s, 1H), 7.76 (s, 1H), 7.33 (s, 1H), 4.37 (br, 1H), 4.23 (br, 2H), 3.28 (br, 2H), 2.85 (t, J = 13.2 Hz, 2H), 2.76 (br, 1H), 2.65 (br, 2H), 2.33~2.39 (m, 6H), 2.10 (s, 3H), 1.72 (br, 2H). ¹⁹FNMR (376 MHz, DMSO-d₆): δ −90.75. |
| 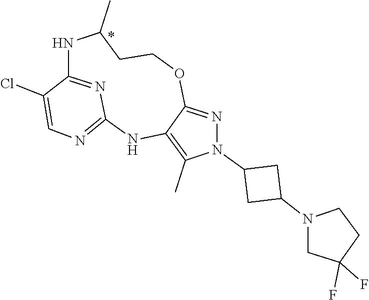 [SMILES: Cc1c2c(nn1C1CC(N3CCC(F)(F)C3)C1)OCCC(C)Nc1nc(ncc1Cl)N2] | Enantiomers 1-4: 14-Chloro-5-[3-(3,3-difluoropyrrolidin-1-yl) cyclobutyl]-4,11-dimethyl-8-oxa-2,5,6,12,16,17-hexaazatricyclo [11.3.1.0^{3,7}]-heptadeca-1(16),3,6,13(17),14-pentaene (E313-E316) Compounds were prepared using procedures similar to those used for preparing E106, using D135 and 3,3-difluoropyrrolidine as starting materials. E313: LC-MS: 454.2 [M + H]⁺. ¹H NMR (400 MHz, CD₃OD): δ 7.72 (s, 1H), 4.44~4.33 (m, 2H), 3.97~3.92 (m, 1H), 2.95-2.90 (m, 3H), 2.77 (t, J = 8.0 Hz, 2H), 2.73~2.57 (m, 3H), 2.44-2.35 (m, 2H), 2.34~2.26 (m, 2H), 2.15 (s, 3H), 1.84~1.74 (m, 2H), 1.30 (d, J = 8.0 Hz, 3H). ¹⁹F 1H (400 MHz, CD₃OD): δ −93.72 (s, 2F). Chiral RT = 2.061 min (chiral method E); ee = 100%. E314: LC-MS: 454.2 [M + H]⁺. ¹H NMR (400 MHz, CD₃OD): δ 7.72 (s, 1H), 4.44~4.33 (m, 2H), 3.97~3.95 (m, 1H), 2.95-2.90 (m, 3H), 2.77 (t, J = 8.0 Hz, 2H), 2.73~2.57 (m, 3H), 2.44-2.37 (m, 2H), 2.35-2.26 (m, 2H), 2.15 (s, 3H), 1.84~1.74 (m, 2H), 1.30 (d, J = 8.0 Hz, 3H). ¹⁹F NMR (400 MHz, CD₃OD): δ −93.72 (s, 2F). Chiral RT = 2.737 min (chiral method E); ee = 100%. E315: LC-MS: 454.2 [M + H]⁺. ¹H NMR (400 MHz, CD₃OD): δ 7.72 |
| | (s, 1H), 4.47~4.35 (m, 3H), 3.96 (br, 1H), 2.97~2.85 (m, 3H), 2.76 |
| | (t, J = 8.0 Hz, 2H), 2.54-2.49 (m, 4H), 2.33~2.23 (m, 2H), 2.17 (s, |
| | 3H), 1.84~1.73 (m, 2H), 1.30 (d, J = 8.0 Hz, 3H). ¹⁹F NMR (400 |
| | MHz, CD₃OD): δ −93.90 (s, 2F). Chiral RT = 3.712 min (chiral |
| | method E); ee = 100%. |
| | E316: LC-MS: 454.2 [M + H]⁺. ¹H NMR (400 MHz, CD₃OD): δ 7.72 |
| | (s, 1H), 4.47~4.34 (m, 3H), 3.96~3.94 (m, 1H), 2.97~2.85 (m, 3H), |
| | 2.76 (t, J = 8.0 Hz, 2H), 2.54-2.50 (m, 4H), 2.34-2.23 (m, 2H), 2.17 |
| | (s, 3H), 1.85~1.74 (m, 2H), 1.30 (d, J = 8.0 Hz, 3H). ¹⁹F NMR (400 |
| | MHz, CD₃OD): δ −93.90 (s, 2F). Chiral RT = 4.949 min (chiral |
| | method E); ee = 100%. |
| 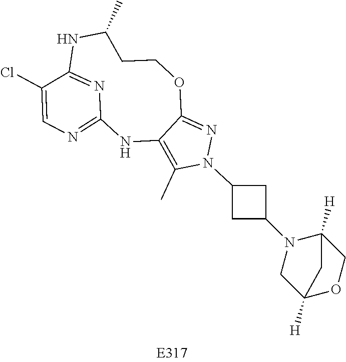 [SMILES: Cc1c2c(nn1C1CC(N3C[C@H]4C[C@@H]3CO4)C1)OCC[C@@H](C)Nc1nc(ncc1Cl)N2] | (11R)-14-Chloro-4,11-dimethyl-5-{3-[(1R,4R)-2-oxa-5-aza bicycle [2.2.1]heptan-5-yl]cyclobutyl}-8-oxa-2,5,6,12,16,17-hexaaza tricyclo[11.3.1.0^{3,7}]heptadeca-1(16),3,6,13(17),14-pentaene (E317) Compound was prepared using procedures similar to those used for preparing E106, by reacting D135 with (1R,4R)-2-oxa-5- azabicyclo[2.2.1]heptane. LC-MS: 446.3[M + H]⁺. ¹H NMR (400 MHz, CD₃OD): δ 7.72 (s, 1H), 4.44~4.37 (m, 4H), 3.99~3.94 (m, 2H), 3.63~3.59 (m, 2H), 3.17~ 3.13 (m, 1H), 2.83~2.80 (m, 1H), 2.69~2.67 (m, 1H), 2.57~2.46 (m, 4H), 2.17 (s, 3H), 1.90~1.87 (m, 1H), 1.81~1.72 (m, 3H), 1.30~1.28 (m, 3H). |
| 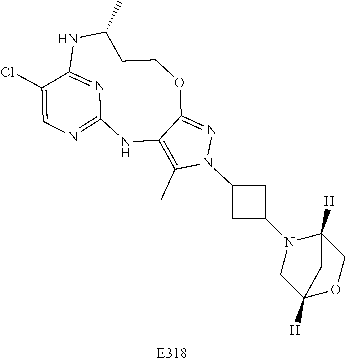 [SMILES: Cc1c2c(nn1C1CC(N3C[C@@H]4C[C@H]3CO4)C1)OCC[C@@H](C)Nc1nc(ncc1Cl)N2] | (11R)-14-Chloro-4,11-dimethyl-5-{3-[(1R,4R)-2-oxa-5-aza bicyclo[2.2.1]heptan-5-yl]cyclobutyl}-8-oxa-2,5,6,12,16,17- hexaazatricyclo[11.3.1.0^{3,7}]heptadeca-1(16),3,6,13(17),14- pentaene (E318) Compound was prepared using procedures similar to those used for preparing E106, by reacting D135 with (1S,4S)-2-oxa-5- azabicyclo[2.2.1]heptane. LC-MS: 446.3 [M + H]⁺. ¹H NMR (400 MHz, CD₃OD): δ 7.72 (s, 1H), 4.45~4.37 (m, 4H), 3.99~3.94 (m, 2H), 3.62~3.61 (m, 2H), 3.18~ 3.12 (m, 1H), 2.84-2.82 (m, 1H), 2.70~2.67 (m, 1H), 2.60~2.47 (m, 4H), 2.17 (s, 3H), 1.90~1.88 (m, 1H), 1.80~1.73 (m, 3H), 1.30~1.28 (m, 3H). |
| 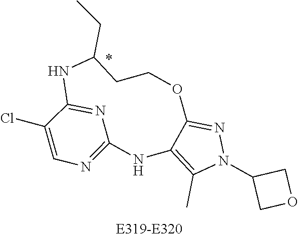 [SMILES: CCC1CCOc2nn(C3COC3)c(C)c2Nc2ncc(Cl)c(n2)N1] | Enantiomers 1-2: 14-Chloro-11-ethyl-4-methyl-5-(oxetan-3-yl)-8- oxa-2,5,6,12,16,17-hexaazatricyclo[11.3.1.0^{3,7}]heptadeca-1(16), 3,6,13(17),14-pentaene (E319-E320) Compounds were prepared using procedures similar to those used for preparing E52, starting from 3-aminopentan-1-ol. E319: LC-MS: 365.2 [M + H]⁺. ¹H NMR (400 MHz, CDCl₃): δ 8.36 (s, 1H), 7.79 (s, 1H), 6.78 (d, J = 7.6 Hz, 1H), 5.42~5.38 (m, 1H), 4.94 (t, J = 6.4 Hz, 1H), 4.84~4.79 (m, 3H), 4.36~4.25 (m, 2H), 3.62~3.59 (m, 1H), 2.08 (s, 3H), 1.80~1.67 (m, 2H), 1.59~1.53 (m, 2H), 0.86 (t, J = 7.2 Hz, 3H). Chiral RT = 3.140 min (chiral method E); ee = 100%. E320: LC-MS: 365.2 [M + H]⁺. ¹H NMR (400 MHz, CDCl₃): δ 8.36 (s, 1H), 7.79 (s, 1H), 6.78 (d, J = 7.6 Hz, 1H), 5.42~5.38 (m, 1H), 4.94 (t, J = 6.4 Hz, 1H), 4.84~4.79 (m, 3H), 4.36~4.25 (m, 2H), 3.62-3.59 (m, 1H), 2.08 (s, 3H), 1.80~1.67 (m, 2H), 1.59~1.53 (m, 2H), 0.86 (t, J = 7.2 Hz, 3H). Chiral RT = 6.195 min (chiral method E); ee = 99.8%. |
| 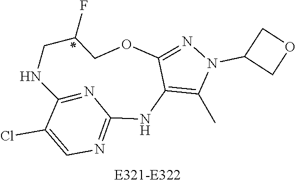 [SMILES: Cc1c2c(nn1C1COC1)OCC(F)CNc1nc(ncc1Cl)N2] | Enantiomers 1-2: 14-Chloro-10-fluoro-4-methyl-5-(oxetan-3-yl)- 8-oxa-2,5,6,12,16,17-hexaazatricyclo[11.3.1.0^{3,7}]heptadeca- 1(16),3,6,13(17),14-pentaene (E321-E322) Compounds were prepared using procedures similar to those used for preparing E52, starting from 3-amino-2-fluorobutan-1-ol. E321: LC-MS: 355.1 [M + H]⁺. ¹H NMR (400 MHz, CDCl₃): δ 7.85 (s, 1H), 6.57 (s, 1H), 5.86 (t, J = 5.6 Hz, 1H), 5.33-5.25 (m, 1H), 5.21 (t, J = 6.4 Hz, 1H), 5.15 (d, J = 6.4 Hz, 1H), 4.92 (t, J = 7.2 Hz, 2H), 4.68~4.59 (m, 2H), 4.57~4.48 (m, 1H), 4.11~3.97 (m, 1H), 3.58- 3.45 (m, 1H), 2.16 (s, 3H). Chiral RT = 1.712 min (chiral method E); ee = 100%. E322: LC-MS: 355.1 [M + H]⁺. ¹H NMR (400 MHz, CDCl₃): δ 7.85 (s, |
| | 1H), 6.76 (s, 1H), 5.91 (t, J = 5.6 Hz, 1H), 5.35~5.25 (m, 1H), 5.21 (t, |
| | J = 6.4 Hz, 1H), 5.16 (t, J = 6.4 Hz, 1H), 4.92 (t, J = 7.2 Hz, 2H), |
| | 4.68~4.48 (m, 3H), 4.11~3.99 (m, 1H), 3.58~3.46 (m, 1H), 2.14 (s, |
| | 3H). Chiral RT = 2.095 min (chiral method E); ee = 99.5%. |
| 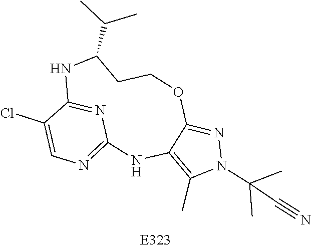 [SMILES: Cc1c2c(nn1C(C)(C)C#N)OCC[C@@H](C(C)C)Nc1nc(ncc1Cl)N2] | 2-[(11S)-14-Chloro-4-methyl-11-(propan-2-yl)-8-oxa-2,5,6,12, 16,17-hexaazatricyclo[11.3.1.0^{3,7}]heptadeca-1(16),3,6,13(17),14- pentaen-5-yl]-2-methylpropanenitrile (E323) Compound was prepared using procedures similar to those used for preparing E43, starting from (S)-3-amino-4-methylpentan-1-ol. LC-MS: 390.2 [M + H]⁺. ¹H NMR (400 MHz, CDCl₃): δ 7.84 (s, 1H), 6.67 (br, 1H), 5.32 (d, J = 7.6 Hz, 1H), 4.48 (dd, J = 11.6, 3.2 Hz, 1H), 4.25 (t, J = 11.2 Hz, 1H), 3.71~3.67 (m, 1H), 2.47 (s, 3H), 2.02~ 1.95 (m, 4H), 1.90 (s, 3H), 1.87~1.82 (m, 2H), 1.01 (dd, J = 6.4, 2.8 Hz, 6H). |
| 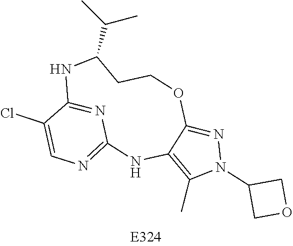 [SMILES: Cc1c2c(nn1C1COC1)OCC[C@@H](C(C)C)Nc1nc(ncc1Cl)N2] | (11S)-14-Chloro-4-methyl-5-(oxetan-3-yl)-11-(propan-2-yl)-8- oxa-2,5,6,12,16,17-hexaazatricyclo[11.3.1.0^{3,7}]heptadeca- 1(16),3,6,13(17),14-pentaene (E324) Compound was prepared using procedures similar to those used for preparing E52, starting from (S)-3-amino-4-methylpentan-1-ol. LC-MS: 379.2 [M + H]⁺. ¹H NMR (400 MHz, CDCl₃): δ 7.81 (s, 1H), 6.38 (s, 1H), 5.30-5.17 (m, 4H), 4.91 (t, J = 7.2 Hz, 2H), 4.56 (dd, J = 11.6, 3.6 Hz, 1H), 4.38 (t, J = 11.2 Hz, 1H), 3.70 (d, J = 5.6 Hz, 1H), 2.15 (s, 3H), 2.03~1.97 (m, 1H), 1.87~1.80 (m, 1H), 1.73~1.67 (m, 1H), 1.01 (d, J = 6.4 Hz, 6H). |
| 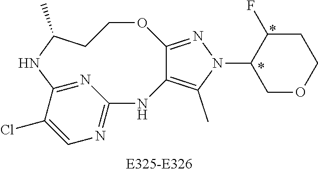 [SMILES: Cc1c2c(nn1C1COCCC1F)OCC[C@@H](C)Nc1nc(ncc1Cl)N2] | Enantiomers 1-2: (11R)-14-Chloro-5-(4-fluorooxan-3-yl)-4,11- dimethyl-8-oxa-2,5,6,12,16,17-hexaazatricyclo[11.3.1.0^{3,7}] heptadeca-1(16),3,6,13(17),14-pentaene (E325-E326) Compounds were prepared using procedures similar to those used for preparing E86, starting from D79. E325: LC-MS: 397.2 [M + H]⁺. ¹H NMR (400 MHz, CD₃OD): δ 7.72 (s, 1H), 5.18~4.91 (m, 1H), 4.40~4.30 (m, 2H), 4.23~4.14 (m, 1H), 4.04~3.90 (m, 3H), 3.79 (t, J = 12.0 Hz, 1H), 3.50 (t, J = 12.0 Hz, 1H), 2.23 (s, 3H), 1.95~1.73 (m, 4H), 1.30 (d, J = 8.0 Hz, 3H). ¹⁹F NMR (376 MHz, CDCl₃): δ −180.06 (s, 1F). Chiral RT = 2.350 min (chiral method C); ee = 100%. E326: LC-MS: 397.2 [M + H]⁺. ¹H NMR (400 MHz, CD₃OD): δ 7.72 |
| | (s, 1H), 5.04~4.89 (m, 1H), 4.40~4.26 (m, 2H), 4.23~4.14 (m, 1H), |
| | 4.06~3.90 (m, 3H), 3.79 (t, J = 12.0 Hz, 1H), 3.51 (t, J = 12.0 Hz, |
| | 1H), 2.23 (s, 3H), 2.08~1.72 (m, 4H), 1.28 (d, J = 8.0 Hz, 3H). ¹⁹F |
| | NMR (376 MHz, CDCl₃): δ −180.45 (s, 1F). Chiral RT = 2.908 min |
| | (chiral method C); ee = 100%. |
| 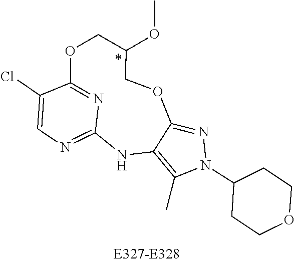 [SMILES: COC1COc2nc(ncc2Cl)Nc2c(nn(C3CCOCC3)c2C)OC1] | Enantiomer 1-2: 14-Chloro-10-methoxy-4-methyl-5-(oxan-4-yl)- 8,12-dioxa-2,5,6,16,17-pentaazatricyclo[11.3.1.0^{3,7}]heptadeca- 1(16),3,6,13(17),14-pentaene (E327-E328) Compounds were prepared using procedures similar to those used for preparing E5, starting from 2-methoxypropane-1,3-diol. E327: LC-MS: 396.1 [M + H]⁺. ¹H NMR (400 MHz, CDCl₃): δ 8.08 (s, 1H), 6.63 (s, 1H), 4.96 (dd, J = 12.4, 2.4 Hz, 1H), 4.50 (dd, J =11.2, 1.6 Hz, 1H), 4.41 (dd, J =11.4, 9.0 Hz, 1H), 4.29 (dd, J = 12.4, 2.4 Hz, 1H), 4.16~4.08 (m, 3H), 3.56~3.51 (m, 2H), 3.47 (s, 3H), 3.43 (dd, J = 9.2, 2.0 Hz, 1H), 2.44~2.25 (m, 2H), 2.23 (s, 3H), 1.85~1.77(m, 2H). Chiral RT = 4.763 min (chiral method A); ee = 100%. E328: LC-MS: 396.2 [M + H]⁺. ¹H NMR (400 MHz, CDCDCl₃): δ 8.08 (s, 1H), 6.55 (s, 1H), 4.96 (dd, J = 12.4, 2.0 Hz, 1H), 4.50 (dd, J = 11.2, 2.0 Hz, 1H), 4.41 (dd, J = 11.2, 9.6 Hz, 1H), 4.29 (dd, J = 12.4, 2.0 Hz, 1H), 4.16~4.07 (m, 3H), 3.56~3.50 (m, 2H), 3.48 (s, 3H), 3.43 (dd, J = 9.2, 2.0 Hz, 1H), 2.44~2.24 (m, 2H), 2.23 (s, 3H), 1.85~1.77 |
| | (m, 2H). Chiral RT = 5.518 min (chiral method A); ee = 100%. |
| 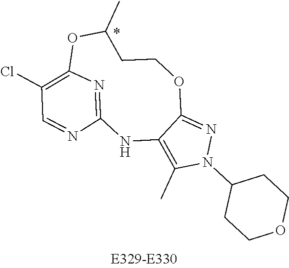 [SMILES: Cc1c2c(nn1C1CCOCC1)OCCC(C)Oc1nc(ncc1Cl)N2] | Enantiomers 1-2: 14-Chloro-4,11-dimethyl-5-(oxan-4-yl)-8,12- dioxa-2,5,6,16,17-pentaazatricyclo[11.3.1.0^{3,7}]heptadeca-1(17), 3,6,13,15-pentaene (E329-E330) Compounds were prepared using procedures similar to those used for preparing E5, starting from 4-((tetrahydro-2H-pyran-2- yl)oxy)butan-2-ol followed by THP de-protection. E329: LC-MS: 380.3 [M + H]⁺. ¹H NMR (400 MHz, CD₃OD): δ 8.00 (s, 1H), 5.05~5.01 (m, 1H), 4.45~4.40 (m, 1H), 4.37~4.23 (m, 2H), 4.07~4.01 (m, 2H), 3.55 (t, J = 16.0 Hz, 2H), 2.23 (s, 3H), 2.20~2.03 (m, 2H), 1.90~1.88 (m, 2H), 1.84~1.73 (m, 2H), 1.39 (d, J = 8.0 Hz, 3H). Chiral RT = 2.221 min (chiral method B); ee = 100%. E330: LC-MS: 380.3 [M + H]⁺. ¹H NMR (400 MHz, CD₃OD): δ 8.00 (s, 1H), 5.05~5.01 (m, 1H), 4.45~4.40 (m, 1H), 4.37~4.23 (m, 2H), 4.07~4.01 (m, 2H), 3.55 (t, J = 16.0 Hz, 2H), 2.23 (s, 3H), 2.20~2.06 (m, 2H), 1.90~1.88 (m, 2H), 1.83~1.73 (m, 2H), 1.38 (d, J = 8.0 Hz, 3H). Chiral RT = 3.219 min (chiral method B); ee = 100%. |
| 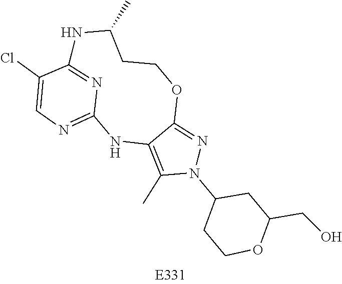 [SMILES: Cc1c2c(nn1C1CCOC(CO)C1)OCC[C@@H](C)Nc1nc(ncc1Cl)N2] | {4-[(11R)-14-Chloro-4,11-dimethyl-8-oxa-2,5,6,12,16,17-hexa azatricyclo[11.3.1.0^{3,7}]heptadeca-1(16),3,6,13(17),14-pentaen-5- yl]oxan-2-yl}methanol (E331) Compound was prepared using procedures similar to those used for OH preparing E46, starting from ethyl 4-((methylsulfonyl)oxy)tetrahydro- 2H-pyran-2-carboxylate and reducing ester group by LiAIH₄ after cyclization. LC-MS: 409.3 [M + H]⁺. ¹H NMR (400 MHz, CDCl₃): δ 7.82 (s, 1H), 6.04 (s, 1H), 5.13~5.11 (m, 1H), 4.49~4.20 (m, 5H), 4.02~3.99 (m, 1H), 3.88~3.86 (m, 1H), 3.66~3.63 (m, 1H), 3.58~3.54 (m, 1H), 2.18 (s, 3H), 2.05~1.79 (m, 7H), 1.34~1.32 (d, J = 6.8 Hz, 3H). |
| 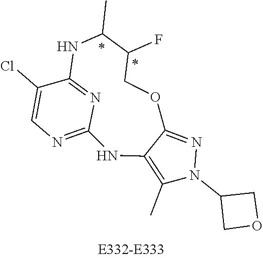 [SMILES: Cc1c2c(nn1C1COC1)OCC(F)C(C)Nc1nc(ncc1Cl)N2] | Enantiomers 1-2: 14-Chloro-10-fluoro-4,11-dimethyl-5-(oxetan- 3-yl)-8-oxa-2,5,6,12,16,17-hexaazatricyclo[11.3.1.0^{3,7}] heptadeca-1(16),3,6,13(17),14-pentaene (E332-E333) Compounds were prepared using procedures similar to those used for preparing E52, starting from D104. E332: 369.2[M + H]⁺. ¹H NMR (400 MHz, CDCₗ₃): δ 7.79 (s, 1H), 6.06 (s, 1H), 5.27-5.14 (m, 1H), 5.17-5.09 (m, 2H), 5.02 (d, J = 7.6 Hz, 1H), 4.84 (t, J = 7.2 Hz, 2H), 4.62~4.39 (m, 3H), 4.39~4.29 (m, 1H), 2.09 (s, 3H), 1.34 (d, J = 7.2 Hz, 3H). Chiral RT = 2.625 min (chiral method E); ee = 100%. E333: 369.2[M + H]⁺. ¹H NMR (400 MHz, CDCl₃): δ 7.79 (s, 1H), 6.09 (s, 1H), 5.29-5.14 (m, 1H), 5.10 (dt, J = 9.6, 6.4 Hz, 2H), 5.02 (d, J = 7.2 Hz, 1H), 4.84 (t, J = 7.2 Hz, 2H), 4.70~4.40 (m, 3H), 4.40~4.20 (m, 1H), 2.09 (s, 3H), 1.34 (d, J = 7.2 Hz, 3H). Chiral RT = 4.992 min (chiral method B); ee = 100%. |
| 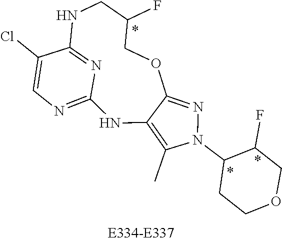 [SMILES: Cc1c2c(nn1C1CCOCC1F)OCC(F)CNc1nc(ncc1Cl)N2] | Enantiomers 1-4: 14-Chloro-10-fluoro-5-(3-fluorooxan-4-yl)-4- methyl-8-oxa-2,5,6,12,16,17-hexaazatricyclo[11.3.1.0^{3,7}] heptadeca-1(16),3,6,13(17),14-pentaene (E334~337) Compounds were prepared using procedures similar to those used for preparing E46, using 3-amino-2-fluoropropan-1-ol and 3- fluorotetrahydro-2H-pyran-4-yl methanesulfonate as starting materials. E334: LC-MS: 401.3 [M + H]⁺. ¹H NMR (400 MHz, CDCl₃): δ 7.84 (s, 1H), 6.63 (s, 1H), 5.86 (s, 1H), 4.91 (dtd, J = 49.2, 9.6, 5.6 Hz, 1H), 4.69~4.45 (m, 3H), 4.26 (dd, J = 10.8, 5.2 Hz, 1H), 4.16~3.98 (m, 3H), 3.64~3.29 (m, 3H), 2.47~2.36 (m, 1H), 2.22 (s, 3H), 1.97~ 1.82 (m, 1H). Chiral RT = 2.688 min (chiral method A); ee = 100%. E335: LC-MS: 401.3[M + H]⁺. ¹H NMR (400 MHz, CDCl₃): δ 7.87 (s, 1H), 6.16 (s, 1H), 5.76 (s, 1H), 4.92~4.71 (m, 1H), 4.66~4.42 (m, 3H), 4.24 (dd, J = 11.2, 5.2 Hz, 1H), 4.20~3.91 (m, 3H), 3.58~3.32 (m, 3H), |
| | 2.52 (ddd, J = 25.2, 12.4, 4.8 Hz, 1H), 2.22 (s, 3H), 2.04~1.91 (m, |
| | 1H). Chiral RT = 3.478 min (chiral method A); ee = 100%. |
| | E336: LC-MS: 401.3[M + H]⁺. ¹H NMR (400 MHz, CDCl₃): δ 7.80 (s, |
| | 1H), 6.21 (s, 1H), 5.70 (t, J = 6.0 Hz, 1H), 4.89~4.61 (m, 1H), 4.61~ |
| | 4.37 (m, 3H), 4.17 (dd, J = 11.0, 5.2 Hz, 1H), 4.12~3.88 (m, 3H), |
| | 3.52~3.21 (m, 3H), 2.45 (ddd, J = 17.2, 12.8, 4.8 Hz, 1H), 2.15 (s, 3H), |
| | 1.99~1.86 (m, 1H). Chiral RT = 3.692 min (chiral method A); ee = |
| | 100%. |
| | E337: LC-MS: 401.3[M + H]⁺. ¹H NMR (400 MHz, CDCl₃): δ 7.79 (s, |
| | 1H), 6.18 (s, 1H), 5.69 (d, J = 6.0 Hz, 1H), 4.98~4.71 (m, 1H), |
| | 4.62~4.40 (m, 3H), 4.19 (dd, J = 11.2, 5.2 Hz, 1H), 4.14~3.88 (m, 3H), |
| | 3.53~3.22 (m, 3H), 2.36 (ddd, J = 25.2, 12.4, 4.8 Hz, 1H), 2.14 (s, |
| | 3H), 1.95~1.77 (m, 1H). Chiral RT = 6.041 min (chiral method A); ee = |
| | 100% |
| 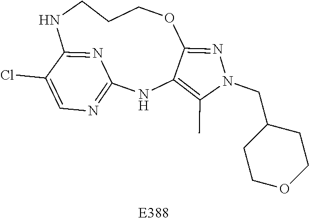 [SMILES: Cc1c2c(nn1CC1CCOCC1)OCCCNc1nc(ncc1Cl)N2] | 14-Chloro-4-methyl-5-(oxan-4-ylmethyl)-8-oxa-2,5,6,12,16,17- hexaazatricyclo[11.3.1.0^{3,7}]heptadeca-1(16),3,6,13(17),14- pentaene (E338) Compound was prepared using procedures similar to those used for preparing E12, starting from (tetrahydro-2H-pyran-4-yl)methyl methanesulfonate. LC-MS: 379.2 [M + H]⁺. ¹H NMR (400 MHz, DMSO-d₆): δ 8.29 (s, 1H), 7.76 (s, 1H), 7.32 (br, 1H), 4.18 (br, 2H), 3.82 (d, J = 9.6 Hz, 2H), 3.72 (d, J = 6.8 Hz, 2H), 3.27~3.17 (m, 4H), 2.12 (s, 3H), 1.99 (br, 1H), 1.70 (br, 2H), 1.39 (d, J = 12.0 Hz, 2H), 1.27~1.16 (m, 2H). |
| 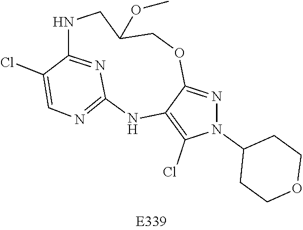 [SMILES: COC1CNc2nc(ncc2Cl)Nc2c(nn(C3CCOCC3)c2Cl)OC1] | 4,14-Dichloro-10-methoxy-5-(oxan-4-yl)-8-oxa-2,5,6,12,16,17- hexaazatricyclo[11.3.1.0^{3,7}]heptadeca-1(16),3,6,13(17),14- pentaene (E339) Compound was prepared using procedures similar to those used for preparing E2 using D13 and D36 as starting materials. LC-MS: 415.1 [M + H]⁺. ¹H NMR (400 MHZ, CDCl₃): δ 9.90 (br, 1H), 7.66 (s, 1H), 6.81 (br, 1H), 4.42~4.38 (m, 1H), 4.33~4.27 (m, 1H), 4.21~4.16 (m, 1H), 4.05~4.02 (m, 2H), 3.90~3.86 (m, 2H), 3.45 (t, J = 12.0 Hz, 3H), 3.39 (s, 3H), 3.31~3.29 (m, 1H), 2.19~2.08 (m, 2H), 1.78~1.75 (m, 2H). |
| 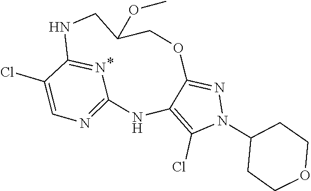 [SMILES: COC1CNc2nc(ncc2Cl)Nc2c(nn(C3CCOCC3)c2Cl)OC1] | Enantiomers 1-2: 4,14-Dichloro-10-methoxy-5-(oxan-4-yl)-8- oxa-2,5,6,12,16,17-hexaazatricyclo[11.3.1.0^{3,7}]heptadeca- 1(16),3,6,13(17),14-pentaene (E340-E341) Compounds were prepared from chiral-separation of E339. E340: LC-MS: 415.1 [M + H]⁺. ¹H NMR (400 MHz, CDCl₃): δ 7.88 (s, 1H), 6.18 (br, 1H), 5.58 (br, 1H), 4.51~4.48 (m, 1H), 4.27~4.26 (m, 2H), 4.24~4.09 (m, 2H), 3.55~3.34 (m, 7H), 2.27~2.20 (m, 2H), 1.87~1.81 (m, 2H), 1.95~1.93 (m, 2H), 1.87~1.80 (m, 2H). Chiral RT = 5.087 min (chiral method A); ee = 100%. E341: LC-MS: 415.1 [M + H]⁺. ¹H NMR (400 MHz, CDCl₃): δ 7.88 (s, 1H), 6.19 (br, 1H), 5.69 (br, 1H), 4.51~4.48 (m, 1H), 4.35~4.26 (m, 2H), 4.12~4.09 (s, 2H), 3.87~3.85 (m, 1H), 3.55~3.49 (m, 2H), 3.43~3.34 (m, 5H), 2.27~2.19 (m, 2H), 1.86~1.81 (m, 2H). Chiral |
| | RT = 5.623 min (chiral method A); ee = 100%. |
| 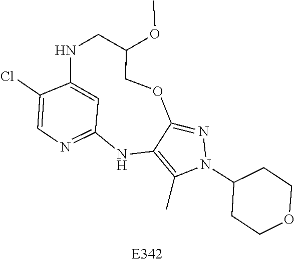 [SMILES: COC1CNc2cc(ncc2Cl)Nc2c(nn(C3CCOCC3)c2C)OC1] | 14-Chloro-10-methoxy-4-methyl-5-(oxan-4-yl)-8-oxa-2,5,6, 12,16-pentaazatricyclo[11.3.1.0^{3,7}]heptadeca-1(16),3,6,13(17), 14-pentaene (E342) Compound was prepared using procedures similar to those used for preparing E5, starting from 2,5-dichloro-4-iodopyridine and 3- amino-2-methoxypropan-1-ol. LC-MS: 394.2 [M + H]⁺. ¹H NMR (400 MHz, CD₃OD): δ 7.59 (s, 1H), 5.53 (s, 1H), 4.44~4.41 (m, 1H), 4.29~4.28 (m, 1H), 4.16~4.11 (m, 1H), 4.07~4.02 (m, 2H), 3.62~3.53 (m, 4H), 3.41(s, 3H), 3.13~3.10 (m, 1H), 2.26 (s, 3H), 2.23~2.10 (m, 2H), 1.83~1.75 (m, 2H). |
| 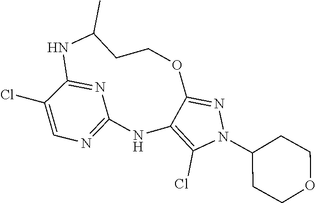 [SMILES: CC1CCOc2nn(C3CCOCC3)c(Cl)c2Nc2ncc(Cl)c(n2)N1] | 4,14-Dichloro-11-methyl-5-(oxan-4-yl)-8-oxa-2,5,6,12,16,17- hexaazatricyclo[11.3.1.0^{3,7}]heptadeca-1(16),3,6,13(17),14- pentaene (E343) Compound was prepared using procedures similar to those used for preparing E13. LC-MS: 399.1 [M + H]⁺. ¹H NMR (400 MHz, CDCl₃): δ 7.85 (s, 1H), 6.29 (br, 1H), 5.16 (br, 1H), 4.64~4.50 (m, 1H), 4.36~4.29 (m, 2H), 4.12~4.02 (m, 3H), 3.55~3.49 (m, 2H), 2.28~2.19 (m, 2H), 1.92~1.81 (m, 4H), 1.34 (d, J = 7.2 Hz, 3H). |
| 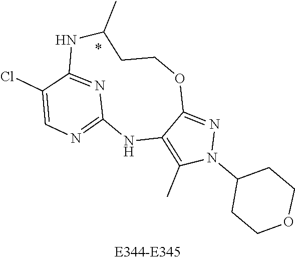 [SMILES: Cc1c2c(nn1C1CCOCC1)OCCC(C)Nc1nc(ncc1Cl)N2] | Enantiomers 1-2: 14-Chloro-4,11-dimethyl-5-(oxan-4-yl)-8-oxa- 2,5,6,12,16-pentaazatricyclo[11.3.1.0^{3,7}]heptadeca-1(16),3,6,13 (17),14-pentaene (E344-E345) Compounds were prepared using procedures similar to those used for preparing E5, starting from 2,5-dichloro-4-iodopyridine. E344: LC-MS: 378.2 [M + H]⁺. ¹H NMR (400 MHz, CD₃OD): δ 7.59 (s, 1H), 5.67 (s, 1H), 4.56~4.51 (m, 1H), 4.31~4.25 (m, 1H), 4.08~4.01 (m, 3H), 3.61~3.53 (m, 3H), 2.26 (s, 3H), 2.24~2.10 (m, 2H), 1.95~1.93 (m, 1H), 1.83~1.74 (m, 2H), 1.68~1.64 (m, 1H), 1.39 (d, J = 7.2 Hz, 3H). Chiral RT = 4.047 min (chiral method A); ee = 100%. E345: LC-MS: 378.2 [M + H]⁺. ¹H NMR (400 MHz, CD₃OD): δ 7.50 (s, 1H), 5.58 (s, 1H), 4.46~4.42 (m, 1H), 4.22~4.16 (m, 1H), 3.98~3.92 (m, 3H), 3.51~3.44 (m, 3H), 2.16 (s, 3H), 2.14~2.00 (m, 2H), 1.90~1.85 (m, 1H), 1.74~1.65 (m, 2H), 1.58~1.54 (m, 1H), 1.30 (d, J = 7.2 Hz, 3H). Chiral RT = 7.342 min (chiral method A); ee = 100%. |
| 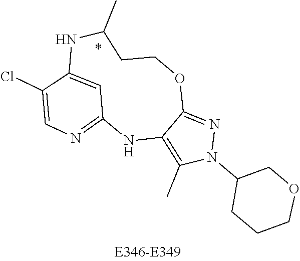 [SMILES: Cc1c2c(nn1C1CCCOC1)OCCC(C)Nc1cc(ncc1Cl)N2] | Enantiomers 1-4: 14-Chloro-4,11-dimethyl-5-(oxan-3-yl)-8-oxa- 2,5,6,12,16-pentaazatricyclo[11.3.1.0^{3,7}]heptadeca-1(16),3,6,13 (17),14-pentaene (E346-E349) Compounds were prepared using procedures similar to those used for preparing E5, starting from 2,5-dichloro-4-iodopyridine D18 and D25. E346: LC-MS: 378.2 [M + H]⁺. ¹H NMR (400 MHz, CD₃OD): δ 7.59 (s, 1H), 5.66 (s, 1H), 4.53~4.49 (m, 1H), 4.17~4.13 (m, 1H), 4.04~3.97 (m, 4H), 3.73~3.68 (m, 1H), 3.60~3.58 (m, 1H), 2.25 (s, 3H), 2.16~1.93 (m, 4H), 1.83~1.81 (m, 2H), 1.40~1.38 (m, 3H). Chiral RT = 2.942 min (chiral method E); ee = 100%. E347: LC-MS: 378.2 [M + H]⁺. ¹H NMR (400 MHz, CD₃OD): δ 7.59 (s, 1H), 5.66 (s, 1H), 4.56~4.49 (m, 1H), 4.19~4.07 (m, 1H), 4.04~3.84 (m, 5H), 3.62-~3.56 (m, 1H), 2.25 (s, 3H), 2.23~2.09 (m, 4H), 2.05~1.80 (m, 2H), 1.40~1.38 (m, 3H). Chiral RT = 3.522 min (chiral method E); ee = 98.3%. |
| | E348: LC-MS: 378.3 [M + H]⁺. ¹H NMR (400 MHz, CD₃OD): δ 7.59 |
| | (s, 1H), 5.66 (s, 1H), 4.54~4.49 (m, 1H), 4.18~4.13 (m, 1H), 4.04~ |
| | 3.91 (m, 4H), 3.73~3.68 (m, 1H), 3.61~3.58 (m, 1H), 2.25 (s, 3H), |
| | 2.16~1.93 (m, 4H), 1.83~1.79 (m, 2H), 1.40~1.38 (m, 3H). Chiral |
| | RT = 4.201 min (chiral method E); ee = 100%. |
| | E349: LC-MS: 378.2 [M + H]⁺. ¹H NMR (400 MHz, CD₃OD): δ 7.59 |
| | (s, 1H), 5.66 (s, 1H), 4.53~4.49 (m, 1H), 4.19~4.14 (m, 1H), |
| | 4.07~4.02 (m, 1H), 3.93~3.84 (m, 2H), 3.62~3.56 (m, 2H), |
| | 3.44~3.40 (m, 1H), 2.25 (s, 3H), 2.21~1.93 (m, 4H), 1.85~1.80 (m, |
| | 2H), 1.40~1.38 (m, 3H). Chiral RT = 7.115 min (chiral method E); |
| | ee = 100%. |
| 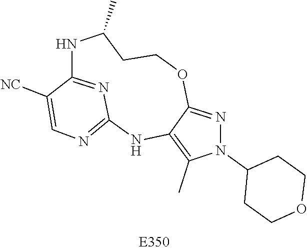 [SMILES: Cc1c2c(nn1C1CCOCC1)OCC[C@@H](C)Nc1nc(ncc1C#N)N2] | (11R)-4,11-Dimethyl-5-(oxan-4-yl)-8-oxa-2,5,6,12,16,17- hexaazatricyclo[11.3.1.0^{3,7}]heptadeca-1(16),3,6,13(17),14- pentaene-14-carbonitrile (E350) Compound was prepared using procedures similar to those used for preparing E69, starting from (R)-3-(methylamino)butan-1-ol. LC-MS: 370.2 [M + H]⁺. ¹H NMR (400 MHz, CDCl₃): δ 8.16 (s, 1H), 6.52 (s, 1H), 5.78 (d, J = 6.8 Hz, 1H), 4.48~4.44 (m, 1H), 4.34 (t, J = 10.4 Hz, 1H), 4.13~4.07 (m, 4H), 3.91~3.87 (m, 1H), 3.55~3.48 (m, 2H), 2.34~2.21 (m, 1H), 2.20 (s, 3H), 1.94~1.76 (m, 4H), 1.30 (d, J = 8.0 Hz, 3H). |
| 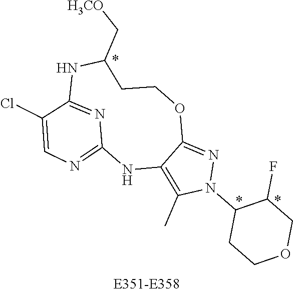 [SMILES: COCC1CCOc2nn(C3CCOCC3F)c(C)c2Nc2ncc(Cl)c(n2)N1] | Enantiomers 1-8: 14-Chloro-5-(3-fluorooxan-4-yl)-11-(methoxy methyl)-4-methyl-8-oxa-2,5,6,12,16,17-hexaazatricyclo [11.3. 1.0^{3,7}]heptadeca-1(16),3,6,13(17),14-pentaene (E351 -E358) Compounds were prepared using procedures similar to those used for preparing E35, starting from D50 and D67. E351: LC-MS: 427.3 [M + H]⁺. ¹H NMR (400 MHz, CDCl₃): δ 7.82 (s, 1H), 6.18 (s, 1H), 5.69 (d, J = 7.2 Hz, 1H), 5.02~4.83 (m, 1H), 4.51~ 4.33 (m, 3H), 4.10~4.07 (m, 3H), 3.52~3.46 (m, 3H), 3.41 (s, 3H), 3.40~3.37 (m, 1H), 2.48~2.36 (m, 1H), 2.21 (s, 3H), 2.07~2.02 (m, 1H),1.95~1.81 (m, 1H), 1.74~1.61 (m, 1H). ¹⁹F NMR (376 MHz, CDCl₃): δ −192.85 (s, 1F). Chiral RT = 2.439 min (chiral method A); ee = 100%. E352: LC-MS: 427.3 [M + H]⁺. ¹H NMR (400 MHz, CDCl₃): δ 7.83 (s, 1H), 6.16 (s, 1H), 5.70 (d, J = 7.2 Hz, 1H), 4.67~4.43 (m, 5H), 4.34~ 4.22 (m, 2H), 4.10~4.01 (m, 2H), 3.51~3.47 (m, 2H), 3.41 (s, 3H), 2.36~2.31 (m, 2H), 2.21 (s, 3H), 2.06~2.01 (m, 1H),1.79~1.71 (m, 1H). ¹⁹F NMR (376 MHz, CDCl₃): δ −231.78 (s, 1F). Chiral RT = 2.831 min (chiral method A); ee = 100%. E353: LC-MS: 427.2 [M + H]⁺. ¹H NMR (400 MHz, CDCl₃): δ 7.83 (s, |
| | 1H), 6.10 (s, 1H), 5.69 (d, J = 7.2 Hz, 1H), 4.68~4.21 (m, 7H), 4.11~ |
| | 4.02 (m, 2H), 3.52~3.48 (m, 2H), 3.41 (s, 3H), 2.59~2.49 (m, 1H), |
| | 2.38~2.28 (m, 1H), 2.21 (s, 3H), 2.07~2.01 (m, 1H), 1.73~1.59 (m, |
| | 1H). ¹⁹F NMR (376 MHz, CDCl₃): δ −232.72 (s, 1F). Chiral RT = |
| | 4.006 min (chiral method A); ee = 100%. |
| | E354: LC-MS: 427.2 [M + H]⁺. ¹H NMR (400 MHz, CDCl₃): δ 7.83 (s, |
| | 1H), 6.13 (s, 1H), 5.68 (d, J = 7.2 Hz, 1H), 4.91~4.72 (m, 1H), 4.49~ |
| | 4.21 (m, 3H), 4.08~4.04 (m, 3H), 3.53~3.46 (m, 3H), 3.41 (s, 3H), |
| | 3.36~3.31 (m, 1H), 2.56~2.46 (m, 1H), 2.21 (s, 3H), 2.07~1.95 (m, |
| | 2H), 1.80~1.59 (m, 1H). ¹⁹F NMR (376 MHz, CDCl₃): δ −193.39 (s, |
| | 1F). Chiral RT = 4.418 min (chiral method A); ee = 100%. |
| | E355: LC-MS: 427.3 [M + H]⁺. ¹H NMR (400 MHz, CDCl₃): δ 7.82 (s, |
| | 1H), 6.37 (s, 1H), 5.68 (d, J = 7.6 Hz, 1H), 4.90~4.72 (m, 1H), 4.49~ |
| | 4.21 (m, 3H), 4.08~4.05 (m, 3H), 3.53~3.45 (m, 3H), 3.41 (s, 3H), |
| | 3.36~3.32 (m, 1H), 2.56~2.45 (m, 1H), 2.21 (s, 3H), 2.07~2.01 (m, |
| | 2H), 1.70~1.59 (m, 1H). ¹⁹F NMR (376 MHz, CDCl₃): δ −193.36 (s, |
| | 1F). Chiral RT = 4.447 min (chiral method A); ee = 100%. |
| | E356: LC-MS: 427.2 [M + H]⁺. ¹H NMR (400 MHz, CDCl₃): δ 7.83 (s, |
| | 1H), 6.14 (s, 1H), 5.69 (d, J = 7.2 Hz, 1H), 4.69~4.21 (m, 7H), 4.12~ |
| | 4.03 (m, 2H), 3.54~3.46 (m, 2H), 3.41 (s, 3H), 2.59~2.51 (m, 1H), |
| | 2.39~2.29 (m, 1H), 2.21 (s, 3H), 2.07~2.01 (m, 1H), 1.73~1.61 (m, |
| | 1H). ¹⁹F NMR (376 MHz, CDCl₃): δ −232.72 (s, 1F). Chiral RT = |
| | 6.029 min (chiral method A); ee = 98.5%. |
| | E357: LC-MS: 427.2 [M + H]⁺. ¹H NMR (400 MHz, CDCl₃): δ 7.82 (s, |
| | 1H), 6.33 (s, 1H), 5.69 (d, J = 7.2 Hz, 1H), 5.02~4.83 (m, 1H), 4.50~ |
| | 4.46 (m, 1H), 4.36~4.24 (m, 2H), 4.14-~4.03 (m, 3H), 3.55~3.45 (m, |
| | 3H), 3.41 (s, 3H), 3.40~3.35 (m, 1H), 2.46~2.36 (m, 1H), 2.21 (s, |
| | 3H), 2.07~2.01 (m, 1H), 1.95~1.89 (m, 1H), 1.81~1.74 (m, 1H). ¹⁹F |
| | NMR (376 MHz, CDCl₃): δ −192.84 (s, 1F). Chiral RT = 7.493 min |
| | (chiral method A); ee = 97.7%. |
| | E358: LC-MS: 427.2 [M + H]⁺. ¹H NMR (400 MHz, CDCl₃): δ 7.83 (s, |
| | 1H), 6.26 (s, 1H), 5.71 (d, J = 7.2 Hz, 1H), 4.68~4.44 (m, 5H), 4.38~ |
| | 4.22 (m, 2H), 4.13~4.03 (m, 2H), 3.53~3.46 (m, 2H), 3.41 (s, 3H), |
| | 2.39~2.28 (m, 2H), 2.21 (s, 3H), 2.09~2.04 (m, 1H), 1.79~1.73 (m, |
| | 1H). ¹⁹F NMR (376 MHz, CDCl₃): δ −231.76 (s, 1F). Chiral RT = |
| | 7.508 min (chiral method A); ee = 100%. |
| 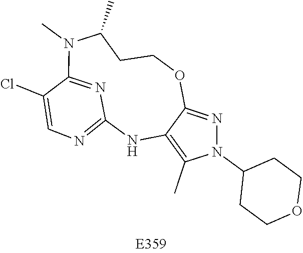 [SMILES: Cc1c2c(nn1C1CCOCC1)OCC[C@@H](C)N(C)c1nc(ncc1Cl)N2] | (11R)-14-Chloro-4,11,12-trimethyl-5-(oxan-4-yl)-8-oxa- 2,5,6,12,16,17-hexaazatricyclo[11.3.1.0^{3,7}]heptadeca- 1(16),3,6,13(17),14-pentaene (E359) Compound was prepared using procedures similar to those used for Preparing E5, starting from (R)-3-(methylamino)butan-1-ol. LC-MS: 393.2 [M + H]⁺. ¹H NMR (400 MHz, DMSO-d₆): δ 8.34 (s, 1H), 7.85 (s, 1H), 4.57~4.46 (m, 1H), 4.32~4.17 (m, 3H), 3.97~3.91 (m, 2H), 3.44 (t, J = 11.6 Hz, 2H), 3.06 (s, 3H), 2.14 (s, 3H), 2.03~1.89 (m, 2H), 1.76~1.63 (m, 4H), 1.17 (d, J = 7.2 Hz, 3H). |
| 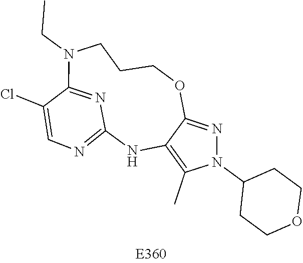 [SMILES: CCN1CCCOc2nn(C3CCOCC3)c(C)c2Nc2ncc(Cl)c1n2] | 14-Chloro-12-ethyl-4-methyl-5-(oxan-4-yl)-8-oxa-2,5,6,12,16,17- hexaazatricyclo[11.3.1.0^{3,7}]heptadeca-1(16),3,6,13(17),14- pentaene (E360) Compound was prepared using procedures similar to those used for preparing E5, starting from 3-(ethylamino)propan-1-ol. LC-MS: 393.2 [M + H]⁺. ¹H NMR (400 MHz, CDCl₃): δ 7.83 (s, 1H), 8.09 (s, 1H), 4.41 (t, J = 4.8 Hz, 2H), 4.12~4.03 (m, 3H), 3.73 (q, J = 7.2 Hz, 2H), 3.55~3.47 (m, 4H), 2.30~2.21 (m, 2H), 2.19 (s, 3H), 1.95~1.89 (m, 2H), 1.79~1.75 (m, 2H), 1.25 (t, J = 6.8 Hz, 3H). |
| 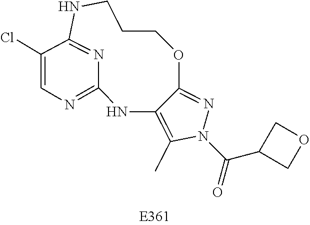 [SMILES: Cc1c2c(nn1C(=O)C1COC1)OCCCNc1nc(ncc1Cl)N2] | 14-Chloro-4-methyl-5-(oxetane-3-carbonyl)-8-oxa-2,5,6,12,16, 17-hexaazatricyclo[11.3.1.0^{3,7}]heptadeca-1(16),3,6,13(17),14- pentaene (E361) To a stirred solution of D142 (90 mg, ca. 0.321 mmol), NMM (81 mg, 0.802 mmol) and oxetane-3-carboxylic acid (40 mg, 0.392 mmol) in DMF (1.5 mL) was added HATU (146 mg, 0.385 mmol). The reaction was stirred at room temperature for 18 hrs. The mixture was quenched with water and the resultant was extracted with EtOAc. The organic phase was dried and filtered. The filtrate was concentrated and the crude was purified by prep-TLC (PE: EtOAc = 1:2) to give the title compound as an off-white solid. LC-MS: 365.1 [M + H]⁺. ¹H NMR (400 MHz, DMSO-d₆): δ 8.81 (s, 1H), 7.83 (s, 1H), 7.46 (t, J = 6.0 Hz, 1H), 4.79~4.88 (m, 5H), 4.29 (t, J = 4.4 Hz, 2H), 3.36~3.27 (m, 2H), 2.45 (s, 3H), 1.74 (br, 2H). |
| 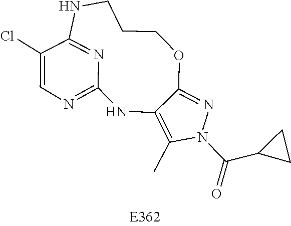 [SMILES: Cc1c2c(nn1C(=O)C1CC1)OCCCNc1nc(ncc1Cl)N2] | 14-Chloro-5-cyclopropanecarbonyl-4-methyl-8-oxa-2,5,6,12,16, 17-hexaazatricyclo[11.3.1.0^{3,7}]heptadeca-1(16),3,6,13(17),14- pentaene (E362) Compound was prepared using procedures similar to those used for preparing E361, starting from D142 and cyclopropanecarboxylic acid. LC-MS: 349.1 [M + H]⁺. ¹H NMR (400 MHz, DMSO-d₆): δ 8.77 (s, 1H), 7.83 (s, 1H), 7.46 (t, J = 6.0 Hz, 1H), 4.38 (t, J = 4.4 Hz, 2H), 3.32 (br, 2H), 3.01~2.97 (m, 1H), 2.40 (s, 3H), 1.76 (br, 2H), 1.05~0.97 (m, 4H). |
| 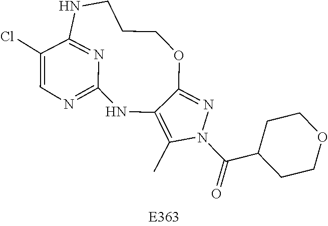 [SMILES: Cc1c2c(nn1C(=O)C1CCOCC1)OCCCNc1nc(ncc1Cl)N2] | 14-Chloro-4-methyl-5-(oxane-4-carbonyl)-8-oxa-2,5,6,12,16,17- hexaazatricyclo[11.3.1.0^{3,7}]heptadeca-1(16),3,6,13(17),14- pentaene (E363) Compound was prepared using procedures similar to those used for preparing E361, starting from D142 and tetrahydro-2H-pyran-4- carboxylic acid. LC-MS: 393.2 [M + H]⁺. ¹H NMR (400 MHz, CDCl₃): δ 7.84 (s, 1H), 6.57 (s, 1H), 5.58 (t, J = 6.2 Hz, 1H), 4.46 (t, J = 4.6 Hz, 2H), 4.07~ 4.04 (m, 2H), 3.83~3.75 (m, 1H), 3.58~3.51 (m, 4H), 2.53 (s, 3H), 1.96~1.88 (m, 6H). |
| 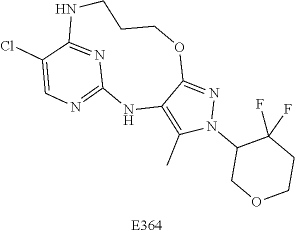 [SMILES: Cc1c2c(nn1C1COCCC1(F)F)OCCCNc1nc(ncc1Cl)N2] | 14-Chloro-5-(4,4-difluorooxan-3-yl)-4-methyl-8-oxa- 2,5,6,12,16,17-hexaazatricyclo[11.3.1.0^{3,7}]heptadeca- 1(16),3,6,13(17),14-pentaene (E364) Compound was prepared using procedures similar to those used for preparing E86 (directly reacting D118 with DAST before cyclization)(racemic compound for E302 and E303). LC-MS: 401.2 [M + H]⁺. ¹H NMR (400 MHz, DMSO-d₆): δ 8.39 (s, 1H), 7.77 (s, 1H), 7.36 (t, J = 5.6 Hz, 1H), 4.65~4.57 (m, 1H), 4.18~ 4.17 (m, 2H), 4.11~3.98 (m, 3H), 3.61~3.56 (m, 1H), 4.00~3.97 (m, 1H), 3.27 (d, J = 5.2 Hz, 1H), 2.24~2.18 (m, 2H), 2.15 (s, 3H), 1.79~1.63 (m, 2H). ¹⁹F NMR (376 MHz, DMSO-d₆): δ −97.52 (d, J = 232.7 Hz, 1F), −114.29 (d, J = 232.7 Hz, 1F). |
| 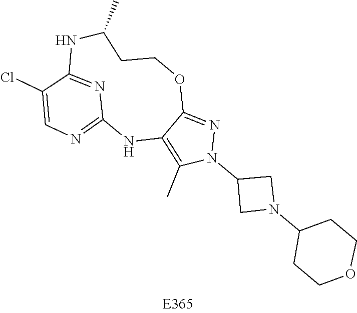 [SMILES: Cc1c2c(nn1C1CN(C3CCOCC3)C1)OCC[C@@H](C)Nc1nc(ncc1Cl)N2] | (11R)-14-Chloro-4,11-dimethyl-5-[1-(oxan-4-yl)azetidin-3-yl]-8- oxa-2,5,6,12,16,17-hexaazatricyclo[11.3.1.0^{3,7}]heptadeca-1(16), 3,6,13(17),14-pentaene (E365) Compound was prepared using procedures similar to those used for preparing E107, starting from D160 and dihydro-2H-pyran-4(3H)- one. LC-MS: 434.2 [M + H]⁺. ¹H NMR (400 MHz, CD₃OD): δ 7.93 (s, 1H), 5.32 (br, 1H), 4.71 (br, 1H), 4.65~4.49 (m, 4H), 4.43~4.41 (m, 1H), 4.09~4.06 (m, 3H), 2.24 (s, 3H), 2.05~2.03 (m, 2H), 1.96~1.83 (m, 2H), 1.59~1.54 (m, 2H), 1.49 (d, J = 7.2 Hz, 3H). |
| 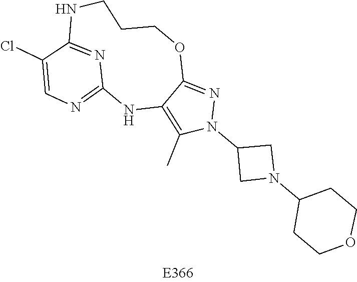 [SMILES: Cc1c2c(nn1C1CN(C3CCOCC3)C1)OCCCNc1nc(ncc1Cl)N2] | 14-Chloro-4-methyl-5-[1-(oxan-4-yl)azetidin-3-yl]-8-oxa-2,5,6,12, 16,17-hexaazatricyclo[11.3.1.0^{3,7}]heptadeca-1(16),3,6,13(17),14- pentaene (E366) Compound was prepared using procedures similar to those used for preparing E107, starting from D33 and dihydro-2H-pyran-4(3H)- one. LC-MS: 420.2 [M + H]⁺. ¹H NMR (400 MHz, CDCl₃): δ 11.47 (s, 1H), 7.65 (s, 1H) 6.52 (t, J = 5.8 Hz, 1H), 5.24 (t, J = 7.2 Hz, 1H), 4.61 (t, J = 6.4 Hz, 2H), 4.35~4.25 (m, 4H), 4.06 (dd, J = 11.6, 3.2 Hz, 2H), 3.58 (t, J = 5.0 Hz, 2H), 3.34 (t, J = 11.2 Hz, 3H), 2.19 (s, 3H), 1.97~1.70 (m, 6H). |
| 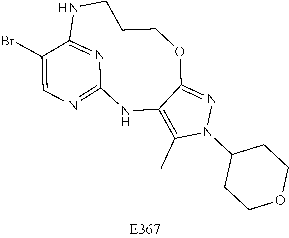 [SMILES: Cc1c2c(nn1C1CCOCC1)OCCCNc1nc(ncc1Br)N2] 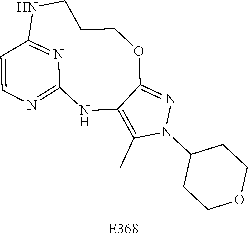 [SMILES: Cc1c2c(nn1C1CCOCC1)OCCCNc1ccnc(n1)N2] | 14-Bromo-4-methyl-5-(oxan-4-yl)-8-oxa-2,5,6,12,16,17-hexaaza tricyclo[11.3.1.0^{3,7}]heptadeca-1(16),3,6,13(17),14-pentaene (E367) 4-Methyl-5-(oxan-4-yl)-8-oxa-2,5,6,12,16,17-hexaazatricyclo [11.3.1.0^{3,7}]heptadeca-1(16),3,6,13(17),14-pentaene (E368) Compounds were prepared using procedures similar to those used for preparing E21, starting from D6 and 5-bromo-2,4- dichloropyrimidine (Br was reduced in the last step to give compound E368) E367: LC-MS: 409.1 [M + H]⁺. ¹H NMR (400 MHz, CDCl₃): δ 7.88 (s, 1H), 6.68 (br, 1H), 5.64 (br, 1H), 4.52~4.27 (m, 2H), 4.20~3.94 (m, 3H), 3.31~3.63 (m, 4H), 2.35~2.17 (m, 5H), 1.92 (br, 3H), 1.79 (d, J = 12.8 Hz, 2H) E368: LC-MS: 331.2 [M + H]⁺. ¹H NMR (400 MHz, CDCl₃): δ 7.59 (d, J = 6.4 Hz, 1H), 5.86 (d, J = 6.4 Hz, 1H), 5.71 (br, 1H), 4.46~4.28 (m, 2H), 4.11 (dd, J = 4.0, 11.6 Hz, 3H), 3.62~3.35 (m, 4H), 2.39~2.14 (m, 5H), 1.95 (br, 2H), 1.78 (d, J = 12.8 Hz, 2H) |
| 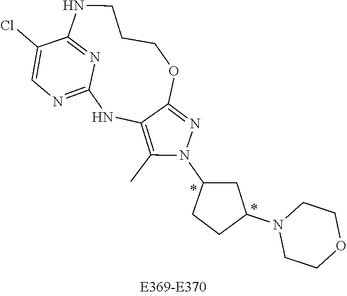 [SMILES: Cc1c2c(nn1C1CCC(N3CCOCC3)C1)OCCCNc1nc(ncc1Cl)N2] | Enantiomers 1-2: 14-Chloro-4-methyl-5-[3-(morpholin-4- yl)cyclopentyl]-8-oxa-2,5,6,12,16,17-hexaazatricyclo[11.3.1.0^{3,7}] heptadeca-1(16),3,6,13(17),14-pentaene (E369-E370) Compounds were prepared using procedures similar to those used for preparing E2, starting from D141. E369: LC-MS: 434.2 [M + H]⁺. ¹H NMR (400 MHz, DMSO-d₆): δ 7.92 (s, 1H), 7.81 (s, 1H), 7.41 (t, J = 5.2 Hz, 1H), 4.63~4.58 (m, 1H), 4.15 (br, 2H), 3.57 (br, 4H), 3.43~3.42 (m, 2H), 2.57~2.55 (m, 1H), 2.39 (br, 4H), 2.17~2.13 (m, 1H), 2.10 (s, 3H), 1.94~1.80 (m, 6H), 1.68~1.60 (m, 1H). Chiral RT = 2.329 min (chiral method A); ee = 100%. E370: LC-MS: 434.3 [M + H]⁺. ¹H NMR (400 MHz, DMSO-d₆): δ 7.92 (s, 1H), 7.81 (s, 1H), 7.41 (t, J = 4.8 Hz, 1H), 4.63~4.58 (m, 1H), 4.15 (br, 2H), 3.57 (br, 4H), 3.43~3.42 (m, 2H), 2.57~2.55 (m, 1H), 2.39 (br, 4H), 2.17~2.13 (m, 1H), 2.10 (s, 3H), 1.94~1.80 (m, 6H), 1.68~1.60 (m, 1H). Chiral RT = 4.420 min (chiral method A); ee = 100%. |
| 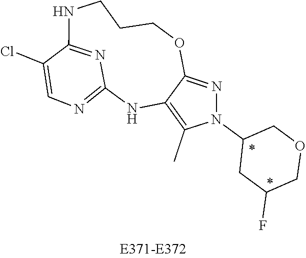 [SMILES: Cc1c2c(nn1C1COCC(F)C1)OCCCNc1nc(ncc1Cl)N2] | Enantiomers 1-2: 14-Chloro-5[5-fluorooxan-3-yl]-4-methyl-8- oxa-2,5,6,12,16,17-hexaazatricyclo[11.3.1.0^{3,7}]heptadeca- 1(16),3,6, 13(17),14-pentaene (E371-E372) Compounds were prepared using procedures similar to those used for preparing E31, starting from D171. E371: LC-MS: 383.2 [M + H]⁺. ¹H NMR (400 MHz, CDCl₃): δ 7.81 (s, 1H), 6.43 (s, 1H), 5.55 (t, J = 6.0 Hz, 1H), 4.89 (d, J = 46.8 Hz, 1H), 4.49~4.42 (m, 1H), 4.36 (t, J = 4.8 Hz, 2H), 4.12 (t, J = 13.2 Hz, 1H), 4.00~3.97 (m, 1H), 3.70 (t, J = 11.2 Hz, 1H), 3.63~3.49 (m, 3H), 2.56~2.38 (m, 2H), 2.22 (s, 3H), 1.92~1.90 (m, 2H). ¹⁹F NMR (376 MHz, CDCl₃): δ −185.98. Chiral RT = 5.373 min (chiral method A); ee = 100%. E372: LC-MS: 383.2 [M + H]⁺. ¹H NMR (400 MHz, CDCl₃): δ 7.81 (s, 1H), 6.23 (s, 1H), 5.55 (t, J = 6.0 Hz, 1H), 4.89 (d, J = 46.4 Hz, 1H), 4.50~4.42 (m, 1H), 4.36 (dd, J = 5.6, 3.6 Hz, 2H), 4.12 (t, J = 13.2 Hz, 1H), 4.00~3.97 (m, 1H), 3.70 (t, J = 10.8 Hz, 1H), 3.64~3.49 (m, 3H), 2.59~2.35 (m, 2H), 2.22 (s, 3H), 1.94~1.89 (m, 2H). ¹⁹F NMR |
| | (376 MHz, CDCl₃): δ −185.99. Chiral RT = 7.239 min (chiral method |
| | A); ee = 100%. |
| 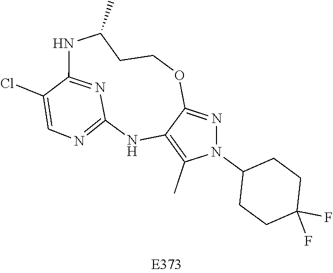 [SMILES: Cc1c2c(nn1C1CCC(F)(F)CC1)OCC[C@@H](C)Nc1nc(ncc1Cl)N2] | (11R)-14-Chloro-5-(4,4-difluorocyclohexyl)-4,11-dimethyl-8-oxa- 2,5,6,12,16,17-hexaazatricyclo[11.3.1.0^{3,7}]heptadeca-1(16),3,6,13 (17),14-pentaene (E373) Compound was prepared using procedures similar to those used for preparing E5, starting from D174. LC-MS: 413.2[M + H]⁺. ¹H NMR (400 MHz, CDCl₃): δ 7.81 (s, 1H), 6.15 (s, 1H), 5.12~5.11 (m, 1H), 4.48~4.44 (m, 1H), 4.35~4.29 (m, 1H), 4.02~3.99 (m, 2H), 2.31~2.21 (m, 4H), 2.19 (s, 3H), 1.97~1.81 (m, 6H), 1.32~1.30(d, J = 6.8 Hz, 3H). |
| 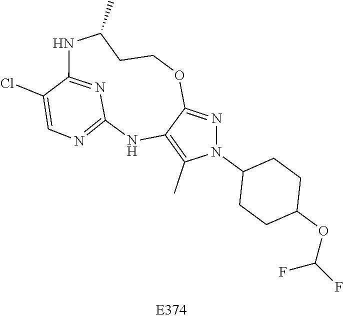 [SMILES: Cc1c2c(nn1C1CCC(OC(F)F)CC1)OCC[C@@H](C)Nc1nc(ncc1Cl)N2] | (11R)-14-Chloro-5-[4-(difluoromethoxy)cyclohexyl]-4,11-di methyl-8-oxa-2,5,6,12,16,17-hexaazatricyclo[11.3.1.0^{3,7}]hepta deca-1(16),3,6,13(17),14-pentaene (E374) Compound was prepared using procedures similar to those used for preparing E5, starting from D176. LC-MS: 443.3 [M + H]⁺. ¹H NMR (400 MHz, CDCl₃): δ 7.80 (s, 1H), 6.54 (br, 1H), 6.45~6.03 (m, 1H), 5.18 (d, J = 7.2 Hz, 1H), 4.47 (dd, J = 3.2, 11.6 Hz, 1H), 4.36~4.25 (m, 1H), 4.23~4.11 (m, 1H), 4.07~ 3.97 (m, 1H), 3.92~3.76 (m, 1H), 2.27~2.04 (m, 6H), 2.02~1.76 (m, 5H), 1.68~1.48 (m, 2H), 1.32 (d, J = 7.2 Hz, 3H). |
| 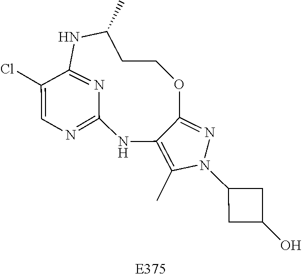 [SMILES: Cc1c2c(nn1C1CC(O)C1)OCC[C@@H](C)Nc1nc(ncc1Cl)N2] | 3-[(11R)-14-Chloro-4,11-dimethyl-8-oxa-2,5,6,12,16,17- hexaazatricyclo[11.3.1.0^{3,7}]heptadeca-1(16),3,6,13(17),14- pentaen-5-yl]cyclobutan-1-ol (E375) Compound was prepared using procedures similar to those used for preparing E5, starting from D177. LC-MS: 365.3 [M + H]⁺. ¹H NMR (400 MHz, CDCl₃): δ 6.85 (br, 1H), 5.30 (d, J = 6.8 Hz, 1H), 4.52~4.49 (m, 1H), 4.38 (t, J = 9.6 Hz, 1H), 4.26 (t, J = 10.8 Hz, 1H), 4.17 (t, J = 6.4 Hz, 1H), 4.05~4.03 (m, 1H), 2.92~2.85 (m, 2H), 2.57~2.46 (m, 2H), 2.17 (s, 3H), 2.11~1.86 (m, 2H), 1.31 (d, J = 6.8 Hz, 3H). |
| 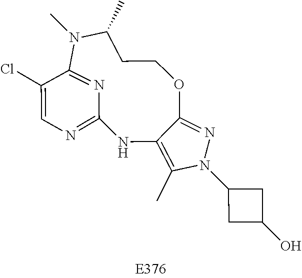 [SMILES: Cc1c2c(nn1C1CC(O)C1)OCC[C@@H](C)N(C)c1nc(ncc1Cl)N2] | 3-[(11R)-14-Chloro-4,11,12-trimethyl-8-oxa-2,5,6,12,16,17- hexaazatricyclo[11.3.1.0^{3,7}]heptadeca-1(16),3,6,13(17),14- pentaen-5-yl] cyclobutan-1-ol (E376) Compound was prepared using procedures similar to those used for preparing E5, starting from D178. LC-MS: 379.3 [M + H]⁺. ¹H NMR (400 MHz, CDCl₃): δ 7.81 (s, 1H), 6.69 (s, 1H), 4.75~4.72 (m, 1H), 4.51~4.48 (m, 1H), 4.42 (t, J = 10.8 Hz, 1H), 4.25 (t, J = 7.2 Hz, 1H), 4.17 (t, J = 6.4 Hz, 1H), 3.18 (s, 3H), 2.91~2.84 (m, 2H), 2.58~2.43 (m, 2H), 2.15 (s, 3H), 1.93~1.87 (m, 1H), 1.73~1.68 (m, 1H), 1.27 (d, J = 6.8 Hz, 3H). |
| 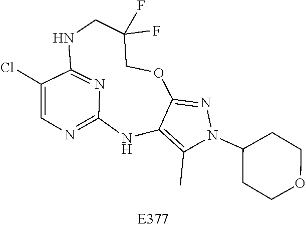 [SMILES: Cc1c2c(nn1C1CCOCC1)OCC(F)(F)CNc1nc(ncc1Cl)N2] | 14-Chloro-10,10-difluoro-4-methyl-5-(oxan-4-yl)-8-oxa- 2,5,6,12,16,17-hexaazatricyclo[11.3.1.0^{3,7}]heptadeca- 1(16),3,6,13(17),14-pentaene (E377) Compound was prepared using procedures similar to those used for preparing E12, starting from tert-butyl (2,2-difluoro-3- hydroxypropyl)carbamate. LC-MS: 401.2 [M + H]⁺. ¹H NMR (400 MHz, CDCl₃): δ 7.90 (d, J = 2.4 Hz, 1H), 6.85 (br, 1H), 5.68 (br, 1H), 4.52 (t, J = 10.8 Hz, 2H), 4.13~4.05 (m, 3H), 4.00~3.92 (m, 2H), 3.51 (t, J = 11.2 Hz, 2H), 2.31~2.19 (m, 5H), 1.80~1.77 (m, 2H). |
| 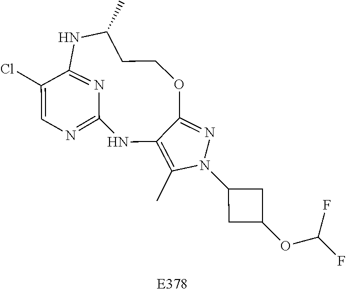 [SMILES: Cc1c2c(nn1C1CC(OC(F)F)C1)OCC[C@@H](C)Nc1nc(ncc1Cl)N2] | (11R)-14-Chloro-5-[3-(difluoromethoxy)cyclobutyl]-4,11- dimethyl-8-oxa-2,5,6,12,16,17-hexaazatricyclo[11.3.1.0^{3,7}] heptadeca-1(16),3,6,13(17),14-pentaene (E378) Compound was prepared using procedures similar to those used for preparing E374, starting from D177. LC-MS: 415.3 [M + H]⁺. ¹H NMR (400 MHz, CDCl₃): δ 7.79~7.77 (m, 1H), 8.70 (br, 1H), 8.21 (t, J = 74 Hz, 1H), 5.25 (d, J = 6.8 Hz, 1H), 4.51~4.46 (m, 2H), 4.40 (d, J = 10.8 Hz, 1H), 4.22~4.16 (m, 1H), 4.06~4.03 (m, 1H), 2.95~2.88 (m, 2H), 2.88~2.74 (m, 2H), 2.17 (s, 3H), 1.94~1.82 (m, 2H), 1.31 (d, J = 6.8 Hz, 3H). |
| 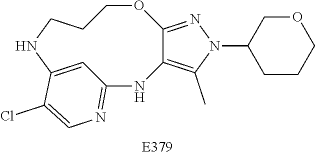 [SMILES: Cc1c2c(nn1C1CCCOC1)OCCCNc1cc(ncc1Cl)N2] | 14-Chloro-4-methyl-5-(oxan-3-yl)-8-oxa-2,5,6,12,16- pentaazatricyclo[11.3.1.0^{3,7}] heptadeca-1 (16),3,6,13(17),14- pentaene (E379) Compound was prepared using procedures similar to those used for preparing E1, starting from 2,5-dichloro-4-iodopyridine and 3- aminopropan-1-ol. LC-MS: 364.1 [M + H]⁺. ¹H NMR (400 MHz, MeOD): δ 7.59 (s, 1H), 5.62 (s, 1H), 4.25 (t, J = 4Hz, 2H), 4.21~4.12 (m, 1H), 3.95~3.88 (m, 2H), 3.65 (t, J = 6 Hz, 1H), 3.48~3.40 (m, 3H), 2.26 (s, 3H), 2.18~2.11 (m, 1H), 2.07~2.01 (m, 1H), 1.88~1.78 (m, 4H). |
| 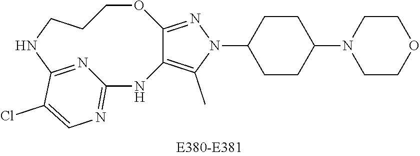 [SMILES: Cc1c2c(nn1C1CCC(N3CCOCC3)CC1)OCCCNc1nc(ncc1Cl)N2] | Isomers1-2: 14-Chloro-4-methyl-5-[4-(morpholin-4- yl)cyclohexyl]-8-oxa-2,5,6,12,16,17-hexaazatricyclo [11.3.1.0^{3,7}]heptadeca-1(16),3,6,13(17),14-pentaene (E380-E381) Compound was prepared using procedures similar to those used for preparing E148, starting from D85. E380: LC-MS: 448.3 [M + H]⁺. ¹H NMR (400 MHz, CDCl₃): δ 7.81 (s, 1H), 6.13 (s, 1H), 5.54 (t, J = 6.0 Hz, 1H), 4.41~4.36 (m, 2H), 3.87~3.79 (m, 1H), 3.76~3.71 (m, 4H), 3.55~3.49 (m, 2H), 2.62~2.56 (m, 4H), 2.41~2.32(m, 1H), 2.19 (s, 3H), 2.11~2.01 (m, 1H), 2.00~1.88 (m, 6H), 1.45~1.34 (m, 3H). Chiral RT = 5.805 min (chiral method F). |
| | E381: LC-MS: 448.3 [M + H]⁺. ¹H NMR (400 MHz, CDCl₃): δ 7.82 (s, |
| | 1H), 6.07 (s, 1H), 5.58-5.51 (m, 1H), 4.43~4.38 (m, 2H), 4.04~3.95 |
| | (m, 1H), 3.79 (t, J = 4.4 Hz, 4H), 3.55~3.50 (m, 2H), 2.52 (br, 4H), |
| | 2.27~2.22 (m, 3H), 2.21 (s, 3H), 2.17~2.09 (m, 2H), 1.96~1.90 (m, |
| | 3H), 1.57~1.42 (m, 3H). Chiral RT = 5.881 min (chiral method F). |
| 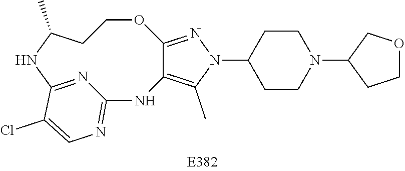 [SMILES: Cc1c2c(nn1C1CCN(C3CCOC3)CC1)OCC[C@@H](C)Nc1nc(ncc1Cl)N2] | (11R)-14-Chloro-4,11-dimethyl-5-[1-(oxolan-3-yl)piperidin-4-yl]- 8-oxa-2,5,6,12,16,17-hexaazatricyclo[11.3.1.0^{3,7}]heptadeca- 1(16),3,6,13(17),14-pentaene (E382) Compound was prepared using procedures similar to those used for preparing E65, starting from D100 and furan-3-one. LC-MS: 448.3 [M + H]⁺. ¹H NMR (400 MHz, CDCl₃): δ 7.80 (s, 1H), 6.23 (s, 1H), 5.12 (d, J = 6.8 Hz, 1H), 4.46~4.42 (m, 1H), 4.31 (t, J = 10.4 Hz, 1H), 4.01~3.94 (m, 2H), 3.90 (dt, J = 7.6 Hz, 2H), 3.80 (q, J = 8.4 Hz, 1H), 3.69 (br, 1H), 3.14~3.09 (m,3H), 2.24 (br, 3H), 2.18 (s, 3H), 2.09~2.06 (m, 1H), 1.92~1.76 (m, 6 H), 1.31 (d, J = 6.8 Hz, 3H). |
| 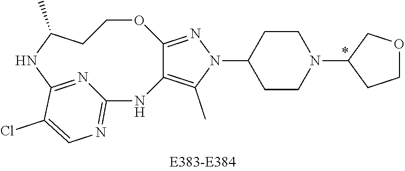 [SMILES: Cc1c2c(nn1C1CCN(C3CCOC3)CC1)OCC[C@@H](C)Nc1nc(ncc1Cl)N2] | Enantiomers 1-2: (11R)-14-Chloro-4,11-dimethyl-5-{1-(oxolan-3- yl)piperidin-4-yl}-8-oxa-2,5,6,12,16,17-hexaazatricyclo [11.3.1.0^{3,7}]heptadeca-1(16),3,6,13(17),14-pentaene (E383-E384) The title compounds E383 and E384 were obtained from chiral separation of E382 (chiral method A). E383: LC-MS: 448.3 [M + H]⁺. ¹H NMR (400 MHz, CDCl₃): δ 7.81 (s, 1H), 6.03 (s, 1H), 5.10 (d, J = 6.8 Hz, 1H), 4.43~4.42 (m, 1H), 4.33 (t, J = 10.4 Hz, 1H), 3.96~3.79 (m, 5H), 3.63 ( t, J = 7.2 Hz, 1H), 3.11~3.08(m, 1H), 3.00 (t, J = 8.0 Hz, 1H), 2.90~2.87 (m, 1H), 2.28~2.23 (m, 3H), 2.21 (s, 3H), 2.13~2.03 (m, 2H), 1.89~1.76 (m, 5H), 1.30 (d, J = 6.8 Hz, 3H). Chiral RT = 13.463 min; ee > 99%. |
| | E384: LC-MS: 448.3 [M + H]⁺. ¹H NMR (400 MHz, CDCl₃): δ 7.81 (s, |
| | 1H), 6.04 (s, 1H), 5.11 (d, J = 6.8 Hz, 1H), 4.43~4.42 (m, 1H), 4.32 |
| | (t, J = 10.4 Hz, 1H), 4.01~3.79 (m, 5H), 3.63 ( t, J = 7.2 Hz, 1H), |
| | 3.11~3.08(m, 1H), 2.99 (t, J = 8.0 Hz, 1H), 2.89~2.86 (m, 1H), |
| | 2.28~2.23 (m,3H), 2.21 (s, 3H), 2.13~2.03 (m, 2H), 1.89~1.76 (m, |
| | 5H), 1.30 (d, J = 6.8 Hz, 3H). Chiral RT = 9.500 min; |
| | ee > 99%. |
| 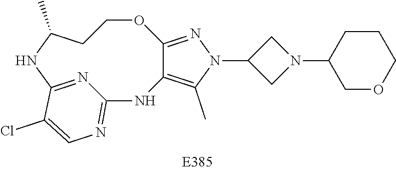 [SMILES: Cc1c2c(nn1C1CN(C3CCCOC3)C1)OCC[C@@H](C)Nc1nc(ncc1Cl)N2] | (11R)-14-Chloro-4,11-dimethyl-5[1-(oxan-3-yl)azetidin-3-yl]-8- oxa-2,5,6,12,16,17-hexaazatricyclo[11.3.1.0^{3,7}]heptadeca- 1(16),3,6,13(17),14-pentaene (E385) Compound was prepared using procedures similar to those used for preparing E65, starting from D160 and dihydro-2H-pyran-3(4H)- one. LC-MS: 434.3 [M + H]⁺. ¹H NMR (400 MHz, DMSO-d₆): δ 8.36 (s, 1H), 7.79 (s, 1H), 6.84 (q, J = 7.2 Hz, 1H), 4.85 (s, 1H), 4.32~4.22 (m, 2H), 3.91~3.64 (m, 6H), 3.20~3.12 (m, 1H), 2.10 (s, 3H), 1.91~1.86 (m, 1H), 1.75~1.58 (m, 3H), 1.47~1.44 (m, 1H), 1.35~1.22 (m, 2H), 1.23 (d, J = 6.8 Hz, 3H). |
| 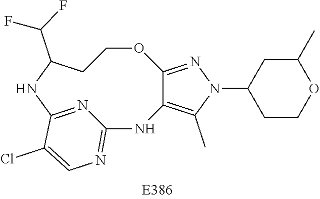 [SMILES: Cc1c2c(nn1C1CCOC(C)C1)OCCC(C(F)F)Nc1nc(ncc1Cl)N2] | 14-Chloro-11-(difluoromethyl)-4-methyl-5-(2-methyloxan-4-yl)- 8-oxa-2,5,6,12,16,17-hexaazatricyclo[11.3.1.0^{3,7}]heptadeca- 1(16),3,6,13(17),14-pentaene (E386) Compound was prepared using procedures similar to those used for preparing E88. LC-MS: 429.2 [M + H]⁺. ¹H NMR (400 MHz, CDCl₃): δ 7.80 (s, 1H), 6.55 (s, 1H), 5.96 (t, J = 16.0 Hz, 1H), 5.36 (d, J = 8.8 Hz, 1H), 4.51~4.18 (m, 5H), 3.86~3.82 (m, 0.5H), 3.54 (m, 0.5H), 2.34 (d, J = 16.0 Hz, 1H), 2.24 (s, 3H), 2.09~1.87 (m, 3H), 1.75~1.66 (m, 2H), 1.28~1.19 (m, 3H). |
| 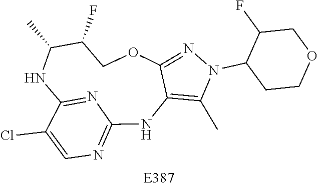 [SMILES: Cc1c2c(nn1C1CCOCC1F)OC[C@@H](F)[C@@H](C)Nc1nc(ncc1Cl)N2] | (10S, 11R)-14-Chloro-10-fluoro-5-(3-fluorooxan-4-yl)-4,11- dimethyl-8-oxa-2,5,6,12,16,17-hexaazatricyclo[11.3.1.0^{3,7}] heptadeca-1(16),3,6,13(17),14-pentaene (E387) Compound was prepared using procedures similar to those used for preparing E63, starting from (2R,3S)-methyl 3-amino-2- hydroxybutanoate hydrochloride and D50. LC-MS: 415.4 [M + H]⁺. ¹H NMR (400 MHz, CDCl₃): δ 7.78 (d, J = 2.6 Hz, 1H), 6.02 (s, 1H), 5.03 (s, 1H), 4.89~4.61 (m, 1H), 4.58~4.36 (m, 3H), 4.33~4.22 (m, 1H), 4.17~4.15 (m, 1H), 4.05~3.98 (m, 2H), 3.42 (t, J = 12.1 Hz, 1H), 3.35~3.24 (m, 1H), 2.48~2.30 (m, 1H), 2.15 (s, 3H), 1.87 (t, J = 14.5 Hz, 1H), 1.34 (dd, J = 7.1, 2.8 Hz, 3H). |
| 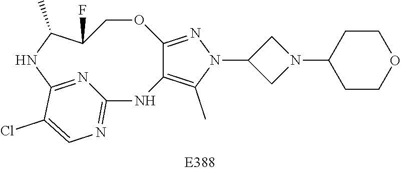 [SMILES: Cc1c2c(nn1C1CN(C3CCOCC3)C1)OC[C@@H](F)[C@@H](C)Nc1nc(ncc1Cl)N2] | (10S, 11R)-14-Chloro-10-fluoro-4,11-dimethyl-5-[1-(oxan-4- yl)azetidin-3-yl]-8-oxa-2,5,6,12,16,17-hexaazatricyclo [11.3.1.0^{3,7}]heptadeca-1(16),3,6,13(17),14-pentaene (E388) Compound was prepared using procedures similar to those used for preparing E390. LC-MS: 452.2 [M + H]⁺. ¹H NMR (400 MHz, CDCl₃): δ 7.86 (s, 1H), 6.28 (s, 1H), 5.15 (d, J = 8.0 Hz, 2H), 4.64~4.31 (m, 6H), 4.22 (br, 1H), 4.13~4.10 (m, 3H), 3.44~3.38 (m, 2H), 3.19 (br, 1H), 2.20 (s, 3H), 1.88~1.82 (m, 4H), 1.43 (t, J = 7.2 Hz, 3H). |
| 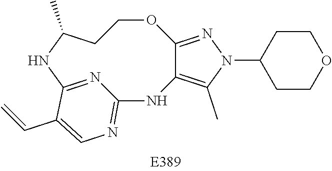 [SMILES: C=Cc1cnc2nc1N[C@H](C)CCOc1nn(C3CCOCC3)c(C)c1N2] | (11R)-14-Ethenyl-4,11-dimethyl-5-(oxan-4-yl)-8-oxa- 2,5,6,12,16,17-hexaazatricyclo[11.3.1.0^{3,7}]heptadeca- 1(16)53,6,13(17),14-pentaene (E389) Compound was prepared using procedures similar to those used for preparing E401, starting from vinylboronic acid pinacol ester. LC-MS: 371.2 [M + H]⁺. ¹H NMR (400 MHz, CDCl₃): δ 7.89 (s, 1H), 6.44 (dd, J = 17.6, 11.2 Hz, 1H), 6.31 (s, 1H), 5.42 (d, J = 17.2 Hz, 1H), 5.18 (d, J = 11.2 Hz, 1H), 4.93 (d, J = 6.8 Hz, 1H), 4.50~4.47 (m, 1H), 4.39~4.33 (m, 1H), 4.15~4.03 (m, 4H), 3.54~3.48 (m, 2H), 2.33~2.23 (m, 2H), 2.21 (s, 3H), 1.93~1.83 (m, 2H), 1.79~1.72 (m, 2H), 1.29 (d, J = 7.2 Hz, 3H). |
| 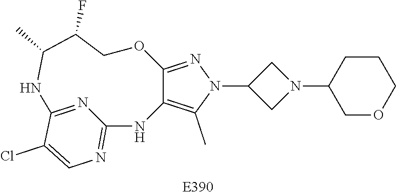 [SMILES: Cc1c2c(nn1C1CN(C3CCCOC3)C1)OC[C@@H](F)[C@@H](C)Nc1nc(ncc1Cl)N2] | (10S, 11R)-14-Chloro-10-fluoro-4,11-dimethyl-5-[1-(oxan-3- yl)azetidin-3-yl]-8-oxa-2,5,6,12,16,17-hexaazatricyclo [11.3.1.0^{3,7}]heptadeca-1(16)53,6,13(17),14-pentaene (E390) Compound E390 was prepared using procedures similar to those used for preparing E63, starting from (2R,3S)-methyl 3-amino-2- hydroxybutanoate hydrochloride. LC-MS: 452.2 [M + H]⁺. ¹H NMR (400 MHz, CDCl₃): δ 7.84 (s, 1H), 6.36 (s, 1H), 5.13 (t, J = 6.2 Hz, 1H), 4.88~4.78 (m, 1H), 4.62~4.51 (m, 3H), 4.41~4.37 (m, 1H), 3.83~3.77 (m, 4H), 3.67~3.55 (m, 2H), 3.50~3.45(m, 1H), 3.29~3.24 (m, 1H), 2.52~2.51 (m, 1H), 2.17 (s, 3H), 1.89~1.88 (m, 1H), 1.78~1.74 (m, 1H), 1.59~1.55 (m, 1H), 1.42 (t, J = 7.2 Hz, 3H), 1.42~1.35 (m, 1H). |
| 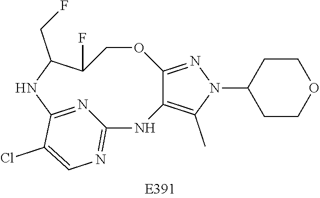 [SMILES: Cc1c2c(nn1C1CCOCC1)OCC(F)C(CF)Nc1nc(ncc1Cl)N2] | 14-Chloro-10-fluoro-11-(fluoromethyl)-4-methyl-5-(oxan-4-yl)-8- oxa-2,5,6,12,16,17-hexaazatricyclo[11.3.1.0^{3,7}]heptadeca- 1(16),3,6,13(17),14-pentaene (E391) Compound E391 was prepared using procedures similar to those used for preparing E63. LC-MS: 415.2 [M + H]⁺. ¹H NMR (400 MHz, CDCl₃): δ 7.90 (s, 1H), 6.23 (s, 1H), 5.60 (dd, J = 6.8, 9.2 Hz, 1H), 5.00~4.38 (m, 6H), 4.16~4.00 (m, 3H), 3.51 (t, J = 12.0 Hz, 2H), 2.44-2.09 (m, 5H), 1.78 (t, J = 14.0 Hz, 2H). |
| 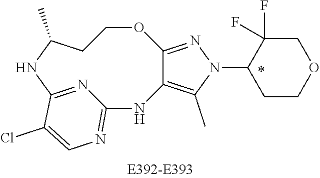 [SMILES: Cc1c2c(nn1C1CCOCC1(F)F)OCC[C@@H](C)Nc1nc(ncc1Cl)N2] | Enantiomers 1-2: (11R)-14-Chloro-5-(3,3-difluorooxan-4-yl)- 4,11-dimethyl-8-oxa-2,5,6,12,16,17-hexaazatricyclo[11.3.1.0^{3,7}] heptadeca-1(16),3,6,13(17),14-pentaene (E392-E393) Compound E392 and E393 were prepared using procedures similar to those used for preparing E414-E415. E392: ¹H NMR (400 MHz, MeOD): δ 7.73 (s, 1 H), 4.57~4.73 (m, 1 H), 4.25~4.46 (m, 2 H), 4.16 (d, J = 12.0 Hz, 1 H), 3.87~4.05 (m, 2 H), 3.56~3.76 (m, 2 H), 2.58~2.81 (m, 1 H), 2.16~2.35 (m, 3 H), 2.03 (d, J = 8.0 Hz, 2 H), 1.68~1.87 (m, 2 H), 1.31 (d, J = 8.0 Hz, 3 H). E393: ¹H NMR (400 MHz, MeOD): δ 7.73 (s, 1 H) 4.58~4.73 (m, 1 H), 4.26~4.46 (m, 2 H), 4.16 (d, J = 12.0 Hz, 1 H), 3.92~4.04 (m, 2 H), 3.58~3.75 (m, 2 H), 2.62~2.81 (m, 1 H), 2.19~2.31 (m, 3 H), |
| | 2.03 (d, J = 8.0 Hz, 2 H), 1.71~1.85 (m, 2 H), 1.31 (d, J = 8.0 Hz, 3 H). |
| 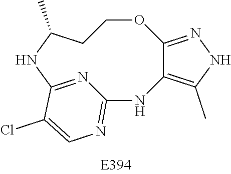 [SMILES: Cc1[nH]nc2c1Nc1ncc(Cl)c(n1)N[C@H](C)CCO2] | (11R)-14-Chloro-4,11-dimethyl-8-oxa-2,5,6,12,16,17- hexaazatricyclo[11.3.1.0^{3,7}]heptadeca-1(16),3,6,13(17),14- pentaene (E394) Compound E394 was prepared using procedures similar to those used for preparing E5, starting from D79. E394: LC-MS: 295 [M + H]⁺. ¹H NMR (400 MHz, DMSO-d₆): δ 11.38 (br, 1H), 8.24 (br, 1H), 7.79 (s, 1H), 6.79-6.81 (br, 1H), 4.20 (t, J = 11.2 Hz, 2H), 3.87 (s, 1H), 2.08 (s, 3H), 1.58~1.73 (m, 2H), 1.23 (d, J = 6.4 Hz, 3H). |
| 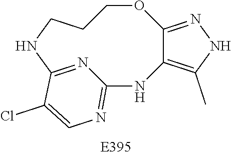 [SMILES: Cc1[nH]nc2c1Nc1ncc(Cl)c(n1)NCCCO2] | 14-Chloro-4-methyl-8-oxa-2,5,6,12,16,17-hexaazatricyclo [11.3.1.0^{3,7}]heptadeca-1(16),3,6,13(17),14-pentaene(E395) Compound E395 was prepared using procedures similar to those used for preparing E5, starting from D33. E394: LC-MS: 281 [M + H]⁺. ¹H NMR (400 MHz, DMSO-d₆): δ 11.38 (brs, 1H), 8.23 (brs, 1H), 7.75 (s, 1H), 7.31-7.28 (brs, 1H), 4.18 (t, J = 4.0 Hz, 2H), 3.21 (s, 2H), 2.33 (s, 3H), 1.71 (s, 2H). |
| 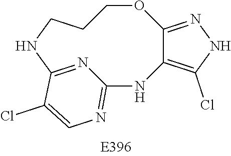 [SMILES: Clc1cnc2nc1NCCCOc1n[nH]c(Cl)c1N2] | 4,14-Dichloro-8-oxa-2,5,6,12,16,17 hexaazatricyclo[11.3.1.0^{3,7}] heptadeca-1(16),3,6,13(17),14-pentaene (E396) Compound was prepared using procedures similar to those used for preparing E13, starting from 5-chloro-4-nitro-1H-pyrazole. LC-MS: 301.1 [M + H]⁺. ¹H NMR (400 MHz, DMSO-d₆): δ 12.73~12.45 (m, 1H), 8.44-8.11 (m, 1H), 7.82 (s, 1H), 7.47~7.42 (m, 1H), 4.22 (s, 2H), 2.50~2.28 (m, 2H), 1.80~1.67 (m, 2H). |
| 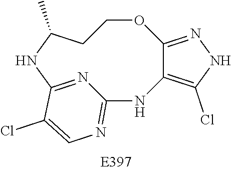 [SMILES: C[C@@H]1CCOc2n[nH]c(Cl)c2Nc2ncc(Cl)c(n2)N1] | (11R)-4,14-Dichloro-11-methyl-8-oxa-2,5,6,12,16,17- hexaazatricyclo[11.3.1.0^{3,7}]heptadeca-1(16),3,6,13(17),14- pentaene (E397) Compound was prepared using procedures similar to those used for preparing E396. LC-MS: 315.1 [M + H]⁺. ¹H NMR (400 MHz, DMSO-d₆): δ 12.73~12.44 (m, 1H), 8.44-8.17 (m, 1H), 7.83 (s, 1H), 6.92 (d, J = 6.4 Hz, 1H), 4.44~3.86 (m, 3H), 1.72 (s, 2H), 1.25 (d, J = 5.2 Hz, 3H). |
| 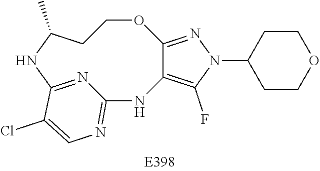 [SMILES: C[C@@H]1CCOc2nn(C3CCOCC3)c(F)c2Nc2ncc(Cl)c(n2)N1] | (11R)-14-Chloro-4-fluoro-11-methyl-5-(oxan-4-yl)-8-oxa- 2,5,6,12,16,17-hexaazatricyclo[11.3.1.0^{3,7}]heptadeca- 1(16),3,6,13(17),14-pentaene (E398) Compound was prepared using procedures similar to those used for preparing E13, starting from 4-nitro-1H-pyrazole. LC-MS: 383.3 [M + H]⁺. ¹H NMR (400 MHz, CDCl₃): δ 7.78 (s, 1H), 5.08 (s, 1H), 4.46~4.18 (m, 2H), 4.15~4.05(m, 1H), 4.03~3.95 (m, 4H), 3.49~3.21 (m, 2H), 2.20~1.95(m, 2H), 1.93~1.65 (m, 4H), 1.27 (d, J = 7.0 Hz, 3H). |
| 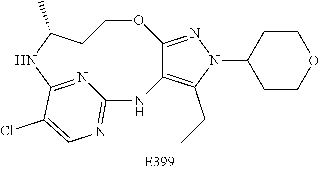 [SMILES: CCc1c2c(nn1C1CCOCC1)OCC[C@@H](C)Nc1nc(ncc1Cl)N2] | (11R)-14-Chloro-4-ethyl-11-methyl-5-(oxan-4-yl)-8-oxa- 2,5,6,12,16,17-hexaazatricyclo[11.3.1.0^{3,7}]heptadeca- 1(16),3,6,13(17),14-pentaene (E399) Compound was prepared using procedures similar to those used for preparing E409, starting from D189 and 4,4,5,5-tetramethyl-2-vinyl- 1,3,2-dioxaborolane. LC-MS: 393.2 [M + H]⁺. ¹H NMR (400 MHz, CDCl₃): δ 7.79 (s, 1H), 6.49 (br, 1H), 5.20 (d, J = 6.4 Hz, 1H), 4.47 (dd, J = 9.2 Hz, 1H), 4.32 (t, J = 10.4 Hz, 1H), 4.11~4.05 (m, 4H), 3.50 ( t, J = 11.2 Hz, 2H), 2.66~2.59 (m, 2H), 2.34-2.29 (m, 2H), 1.89~1.62 (m, 4 H), 1.32 (d, J = 7.2 Hz, 3H), 1.18 ( t, J = 7.6 Hz, 3H). |
| 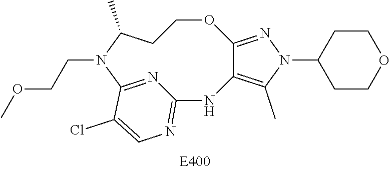 [SMILES: COCCN1c2nc(ncc2Cl)Nc2c(nn(C3CCOCC3)c2C)OCC[C@H]1C] | (11R)-14-Chloro-12-(2-methoxyethyl)-4,11-dimethyl-5-(oxan-4- yl)-8-oxa-2,5,6,12,16,17-hexaazatricyclo[11.3.1.0^{3,7}]heptadeca- 1(16),3,6,13(17),14-pentaene (E400) Compound was prepared using procedures similar to those used for preparing E359, starting from (R)-3-aminobutan-1-ol. LC-MS: 437.3 [M + H]⁺. ¹H NMR (400 MHz, CDCl₃): δ 7.86 (s, 1H), 6.21 (s, 1H), 4.76~4.72 (m, 1H), 4.43~4.39 (m, 2H), 4.12~4.06 (m, 3H), 4.00~3.94 (m, 1H), 3.78~3.71 (m, 1H), 3.59~3.47 (m, 4H), 3.35 (s, 3H), 2.30~2.22 (m, 2H), 2.18 (s, 3H), 1.88~1.69 (m, 4H), 1.26 (d, J = 7.2 Hz, 3H). |
| 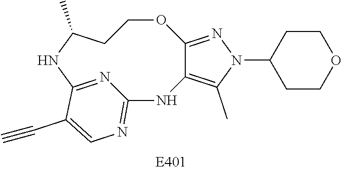 [SMILES: C#Cc1cnc2nc1N[C@H](C)CCOc1nn(C3CCOCC3)c(C)c1N2] | (11R)-14-Ethynyl-4,11-dimethyl-5-(oxan-4-yl)-8-oxa- 2,5,6,12,16,17-hexaazatricyclo[11.3.1.0^{3,7}]heptadeca- 1(16),3,6,13(17),14-pentaene (E401) Compound was prepared using procedures similar to those used for preparing E1, starting from 5-bromo-2,4-dichloropyrimidine and ethynyltrimethylsilane. LC-MS: 369.2 [M + H]⁺. ¹H NMR (400 MHz, CDCl₃): δ 8.02 (s, 1H), 6.44 (br, 1H), 5.31 (d, J = 7.2 Hz, 1H), 4.51~4.47 (m, 1H), 4.33 (t, J = 10.6 Hz, 1H), 4.12~4.04 (m, 4H), 3.54~3.48 (m, 2H), 3.41 (s, 1H), 2.37~2.17 (m, 2H), 2.21 (s, 3H), 1.94~1.75 (m, 4H), 1.30 (d, J = 7.0 Hz, 3H). |
| 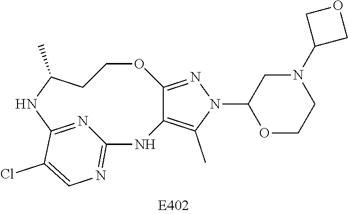 | (11R)-14-Chloro-4,11-dimethyl-5-[4-(oxetan-3-yl)morpholin-2- yl]-8-oxa-2,5,6,12,16,17-hexaazatricyclo[11.3.1.0^{3,7}]heptadeca- 1(16),3,6,13(17),14-pentaene (E402) Compound was prepared using procedures similar to those used for preparing E1, starting from morpholin-2-ol hydrochloride. LC-MS: 295.2 [M + H]⁺. ¹H NMR (400 MHz, CDCl₃): δ 7.79 (s, 1H), 6.62 (br, 1H), 5.29-5.26 (m, 2H), 4.72~4.67 (m, 2H), 4.65~4.60 (m, 2H), 4.48~4.36 (m, 2H), 4.07~4.03 (dd, J = 11.6 Hz, 3.2 Hz, 2H), 3.92~3.86 (m, 1H), 3.67~3.62 (m, 1H), 2.88~2.80 (m, 2H), 2.63 (d, J = 11.2 Hz, 1H), 2.27 (s, 3H), 2.25-2.21 (m, 1H), 1.93~1.65 (m, 2H), 1.30 (d, J = 6.8 Hz, 3H). |
| 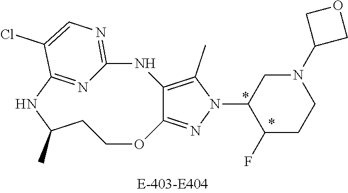 [SMILES: Cc1c2c(nn1C1CN(C3COC3)CCC1F)OCC[C@@H](C)Nc1nc(ncc1Cl)N2] | Enantiomers 1-2: (11R)-14-Chloro-5-[4-fluoro-1-(oxetan-3- yl)piperidin-3-yl]-4,11-dimethyl-8-oxa-2,5,6,12,16,17- hexaazatricyclo[11.3.1.0^{3,7}]heptadeca-1(16),3,6,13(17),14- pentaene (E403-E404) Compound was prepared using procedures similar to those used for preparing E325, starting from tert-butyl 3-bromo-4-oxopiperidine-1- carboxylate. E403: LC-MS: 452.3 [M + H]⁺. ¹H NMR (400 MHz, CDCl₃): δ 7.81 (s, 1H), 6.43 (br, 1H), 5.15 (br, 1H), 4.88~4.58 (m, 5H), 4.46~4.44 (m, 1H), 4.32~4.17 (m, 2H), 4.02~3.98 (m, 1H), 4.62~3.59 (m, 1H), 2.93 (t, J = 16 Hz, 1H), 2.82 (d J = 16 Hz, 1H), 2.63~2.57 (m, 1H), 2.26~2.23 (m, 1H), 2.23 (s, 3H), 2.08~1.83 (m, 4H), 1.30 (d, J = 8 Hz, 3H). |
| | Chiral RT = 3.995 min (chiral method B); ee > 99%. |
| | E403: LC-MS: 452.3 [M + H]⁺. ¹H NMR (400 MHz, CD₃OD): δ 7.72 (s, |
| | 1H), 4.98~4.83 (m, 1H), 4.71~4.57(m, 4H), 4.39~4.23(m, 3H), 3.99- |
| | 3.95(m, 1H), 3.61~3.58 (m, 1H), 2.89~2.83 (m, 2H), 2.43 (t, J = 24 |
| | Hz, 1H), 2.27~2.24 (m, 1H), 2.23 (s, 3H), 2.09~1.02 (m, 1H), 1.93- |
| | 1.77 (m, 3H), 1.28 (d, J = 8 Hz, 3H). Chiral RT = 5.028 min (chiral |
| | method B); ee > 99%. |
| 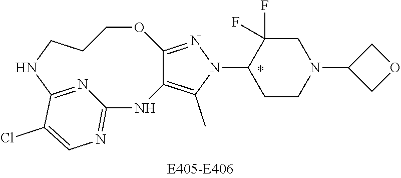 [SMILES: Cc1c2c(nn1C1CCN(C3COC3)CC1(F)F)OCCCNc1nc(ncc1Cl)N2] | Enantiomers 1-2: 14-Chloro-5-[3,3-difluoro-1-(oxetan-3- yl)piperidin-4-yl]-4-methyl-8-oxa-2,5,6,12,16,17- hexaazatricyclo[11.3.1.0^{3,7}]heptadeca-1(16),3,6,13(17),14- pentaene (E405-E406) Compound was prepared using procedures similar to those used for preparing E446. E405: LC-MS: 456.3 [M + H]⁺. ¹H NMR (400 MHz, CDCl₃): δ 7.81 (s, 1H), 6.22 (s, 1H), 5.54 (t, J = 6.2 Hz, 1H), 4.71~4.63 (m, 4H), 4.39 (t, J = 4.6 Hz, 2H), 4.23~4.14 (m, 1H), 3.78~3.71 (m, 1H), 3.52~3.50 (m, 2H), 3.11~3.03 (m, 2H), 2.74~2.66 (m, 1H), 2.50~2.40 (m, 1H), 2.33~2.28 (m, 1H), 2.22 (s, 3H), 2.13~2.08 (m, 1H), 1.98~1.85 (m, 2H). Chiral RT = 5.961 min (chiral method A); ee > 99%. |
| | E406: LC-MS: 456.3 [M + H]⁺. ¹H NMR (400 MHz, CDCl₃): δ 7.82 (s, |
| | 1H), 6.39 (s, 1H), 5.56 (s, 1H), 4.69~4.64 (m, 4H), 4.45~4.39 (m, |
| | 2H), 4.25~4.14 (m, 1H), 3.78~3.69 (m, 1H), 3.52~3.44 (m, 2H), |
| | 3.08~3.06 (m, 2H), 2.74~2.66 (m, 1H), 2.49~2.40 (m, 1H), |
| | 2.31~2.28 (m, 1H), 2.22 (s, 3H), 2.12~2.08 (m, 1H), 1.92~1.78 (m, |
| | 2H). Chiral RT = 9.815 min (chiral method A); ee > 99%. |
| 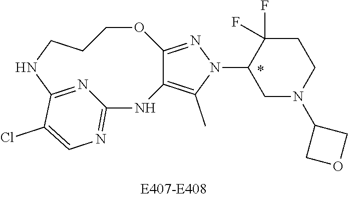 [SMILES: Cc1c2c(nn1C1CN(C3COC3)CCC1(F)F)OCCCNc1nc(ncc1Cl)N2] | Enantiomers 1-2: 14-Chloro-5-[4,4-difluoro-1-(oxetan-3- yl)piperidin-3-yl]-4-methyl-8-oxa-2,5,6,12,16,17- hexaazatricyclo[11.3.1.0^{3,7}]heptadeca-1(16),3,6,13(17),14- pentaene (E407-E408) Compound was prepared using procedures similar to those used for preparing E442-E443. E407: LC-MS: 456.3 [M + H]⁺. ¹H NMR (400 MHz, CDCl₃): δ 7.81 (s, 1H), 6.25 (s, 1H), 5.55 (s, 1H), 4.69 (q, J = 6.8 Hz, 2H), 4.61~4.58 (m, 2H), 4.40~4.31 (m, 3H), 3.73~3.65 (m, 1H), 3.53~3.45 (m, 2H), 3.04-2.75 (m, 3H), 2.28~2.06 (m, 6H), 1.97~1.83 (m, 2H). Chiral RT = 5.296 min (chiral method A); ee > 99%. E408: LC-MS: 456.3 [M + H]⁺. ¹H NMR (400 MHz, CDCl₃): δ 7.82 (s, 1H), 6.15 (s, 1H), 5.54 (s, 1H), 4.69 (q, J = 6.8 Hz, 2H), 4.61~4.58 |
| | (m, 2H), 4.40~4.31 (m, 3H), 3.73~3.64 (m, 1H), 3.53~3.47 (m, 2H), |
| | 3.07~2.75 (m, 3H), 2.28~2.09 (m, 6H), 2.00~1.82 (m, 2H). Chiral |
| | RT = 7.803 min (chiral method A); ee > 99%. |
| 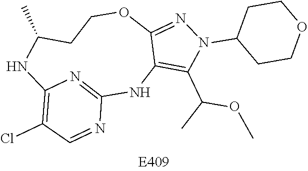 [SMILES: COC(C)c1c2c(nn1C1CCOCC1)OCC[C@@H](C)Nc1nc(ncc1Cl)N2] | (11R)-14-Chloro-4-(1-methoxyethyl)-11-methyl-5-(oxan-4-yl)-8- oxa-2,5,6,12,16,17-hexaazatricyclo[11.3.1.0^{3,7}]heptadeca- 1(17),3,6,13,15-pentaene (E409) Compound was prepared using procedures similar to those used for preparing E416, starting from D189. LC-MS: 423.3 [M + H]⁺. ¹H NMR (400 MHz, CDCl₃): δ 7.81 (s, 1H), 6.70 (br, 1H), 5.14 (br, 1H), 4.55~4.45 (m, 2H), 4.32~4.21 (m, 2H), 4.12~4.07 (m, 3H), 3.50 (t, J = 24.0 Hz, 2H), 3.27 (s, 3H), 2.37~2.27 (m, 2H), 2.00~1.72 (m, 4H), 1.51 (d, J = 8.0 Hz, 3H), 1.34 ( d, J = 8.0 Hz, 3H). |
| 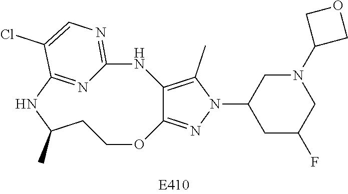 [SMILES: Cc1c2c(nn1C1CC(F)CN(C3COC3)C1)OCC[C@@H](C)Nc1nc(ncc1Cl)N2] | (11R)-14-Chloro-5-[5-fluoro-1-(oxetan-3-yl)piperidin-3-yl]-4,11- dimethyl-8-oxa-2,5,6,12,16,17 hexaazatricyclo[11.3.1.0^{3,7}] heptadeca-1(16),3,6,13(17),14-pentaene (E410) Compound was prepared using procedures similar to those used for preparing E86, starting from tert-butyl 5-hydroxy-5,6- dihydropyridine-1(2H)-carboxylate. LC-MS: 452.3 [M + H]⁺. |
| 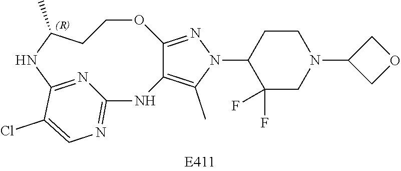 | (11R)-14-Chloro-5-[3,3-difluoro-1-(oxetan-3-yl)piperidin-4-yl]- 4,11-dimethyl-8-oxa-2,5,6,12,16,17-hexaazatricyclo[11.3.1. 0^{3,7}]heptadeca-1(16),3,6,13(17),14-pentaene (E411) Compound was prepared using procedures similar to those used for preparing E96, starting from D182. LC-MS: 470 [M + H]⁺. ¹H NMR (400 MHz, CD₃OD): δ 7.73 (s, 1H), 4.73~4.67 (m, 2H), 4.65~4.58 (m, 2H), 4.53~4.47 (m, 1H), 4.41~4.29 (m, 2H), 3.94~3.91 (m, 1H), 3.76~3.69 (m, 1H), 3.12~3.97 (m, 2H), 2.73~2.64 (m, 1H), 2.50~2.41 (m, 1H), 2.33~2.30 (m, 1H), 2.23 (s, 3H), 2.07~2.03 (m, 1H), 1.83~1.76 (m, 2H), 1.29~1.27 (d, J = 8.0 Hz, 3H). |
| 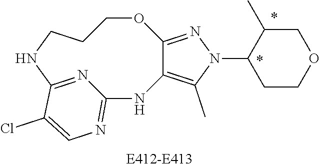 [SMILES: Cc1c2c(nn1C1CCOCC1C)OCCCNc1nc(ncc1Cl)N2] | Isomers 1-2: 14-Chloro-4-methyl-5-[(3R,4S)-3-methyloxan-4-yl]- 8-oxa-2,5,6,12,16,17-hexaazatricyclo[11.3.1.0^{3,7}]heptadeca- 1(16),3,6,13(17),14-pentaene (E412-E413) Compounds were prepared using procedures similar to those used for preparing E1, starting from D187. E412: LC-MS: 379.2 [M + H]⁺. ¹H NMR (400 MHz, MeOD): δ 7.71 (s, 1H), 4.30 (t, J = 4.4 Hz, 2H), 4.05~4.02 (m, 1H), 3.96~3.93 (m, 1H), 3.92~3.82(m, 1H), 3.56 (t, J = 10.8 Hz, 1H), 3.42~3.38 (m, 2H), 3.19 (t, J = 11.2 Hz, 1H), 2.30~2.24 (m, 1H), 2.28 (s, 3H), 2.25-2.20 (m, 1H), 2.17~1.78 (m, 3H), 0.64 (d, J = 6.8 Hz, 3H). E413: LC-MS: 379.2 [M + H]⁺. ¹H NMR (400 MHz, MeOD): δ 7.70 (s, |
| | 1H), 4.45~4.41 (m, 1H), 4.33 (t, J = 4.4 Hz, 2H), 4.20~4.15 (m, 1H), |
| | 3.84~3.82(m, 1H), 3.56 (t, J = 10.8 Hz, 2H), 3.40 (t, J = 5.2 Hz, 2H), |
| | 2.48~2.44 (m, 1H), 2.23 (s, 3H), 2.09~2.07 (m, 1H), 1.87~1.76 (m, |
| | 3H), 0.83 (d, J = 6.8 Hz, 3H). |
| 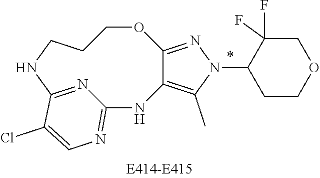 [SMILES: Cc1c2c(nn1C1CCOCC1(F)F)OCCCNc1nc(ncc1Cl)N2] | Enantiomers 1-2: 14-Chloro-5-[(4R)-3,3-difluorooxan-4-yl]-4- methyl-8-oxa-2,5,6,12,16,17-hexaazatricyclo[11.3.1.0^{3,7}] heptadeca- 1(16),3,6,13(17),14-pentaene (E414-E415) Compounds were prepared using procedures similar to those used for preparing E1, starting from D166. E414: LC-MS: 401.3 [M + H]⁺. ¹H NMR (400 MHz, CDCl₃): δ 7.82 (s, 1H), 6.17 (br, 1H), 5.55 (t, J = 6.6 Hz, 1H), 4.40 (t, J = 4.8 Hz, 2H), 4.38~4.30 (m, 2H), 4.14~4.06 (m, 1H), 3.71~3.57 (m, 2H), 3.54~3.50 (m, 2H), 2.75-2.65 (m, 1H), 2.23 (s, 3H), 2.20~2.12 (m, 1H), 1.99~1.84 (m, 2H). Chiral RT = 0.942 min (chiral method E); ee > 99%. E415: LC-MS: 401.3 [M + H]⁺. ¹H NMR (400 MHz, CDCl₃): δ 7.82 (s, |
| | 1H), 6.09 (s, 1H), 5.53 (t, J = 6.0 Hz, 1H), 4.40 (t, J = 4.6 Hz, 2H), |
| | 4.38~4.30 (m, 2H), 4.14~4.06 (m, 1H), 3.71~3.57 (m, 2H), |
| | 3.54~3.48(m, 2H), 2.75-2.66 (m, 1H), 2.23 (s, 3H), 2.20~2.13 (m, |
| | 1H), 1.98~1.84 (m, 2H). Chiral RT = 1.182 min (chiral method E); |
| | ee > 95%. |
| 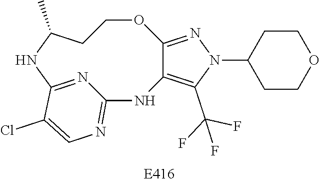 [SMILES: C[C@@H]1CCOc2nn(C3CCOCC3)c(C(F)(F)F)c2Nc2ncc(Cl)c(n2)N1] | (11R)-14-Chloro-11-methyl-5-(oxan-4-yl)-4-(trifluoromethyl)-8- oxa-2,5,6,12,16,17-hexaazatricyclo[11.3.1.0^{3,7}]heptadeca- 1(16),3,6,13(17),14-pentaene (E416) Compound was prepared using procedures similar to those used for preparing E1, starting from D190. LC-MS: 433.2 [M + H]⁺. ¹H NMR (400 MHz, CD₃OD): δ 7.78 (s, 1H), 4.42~4.39 (m, 2H), 4.37~4.29 (m, 1H), 4.09~4.02 (m, 2H), 3.98~3.89 (m, 1H), 3.58~3.50 (m, 2H), 2.37~2.29 (m, 1H), 2.22~2.12 (m, 1H), 1.92~1.76 (m, 4H), 1.31 (d, J = 8.0 Hz, 3H). |
| 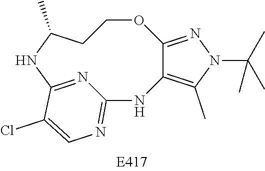 [SMILES: Cc1c2c(nn1C(C)(C)C)OCC[C@@H](C)Nc1nc(ncc1Cl)N2] | (11R)-5-tert-Butyl-14-chloro-4,11-dimethyl-8-oxa-2,5,6,12,16,17- hexaazatricyclo[11.3.1.0^{3,7}]heptadeca-1(16),3,6,13(17),14- pentaene (E417) Compound was prepared using procedures similar to those used for preparing E420, starting from ethyl 2-(2-methyl-1,3-dioxolan-2- yl)acetate and tert-butylhydrazine hydrochloride. LC-MS: 351.2 [M + H]⁺. ¹H NMR (400 MHz, CDCl₃): δ 7.80 (s, 1H), 6.23 (s, 1H), 5.12 (d, J = 7.2 Hz,1H), 4.49~4.44 (m, 1H), 4.33 (t, J = 10.0 Hz, 1H), 4.05~4.02(m, 1H), 2.33 (s, 3H), 1.92~1.57 (m, 2H), 1.57 (m, 9H), 1.31 (d, J = 7.2 Hz, 3H). |
| 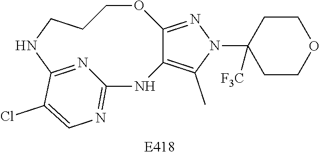 [SMILES: Cc1c2c(nn1C1(C(F)(F)F)CCOCC1)OCCCNc1nc(ncc1Cl)N2] | 14-Chloro-4-methyl-5-[4-(trifluoromethyl)oxan-4-yl]-8-oxa- 2,5,6,12,16,17-hexaazatricyclo[11.3.1.0^{3,7}]heptadeca- 1(16),3,6,13(17),14-pentaene (E418) Compound was prepared using procedures similar to those used for preparing E420, starting from TMSCF₃. LC-MS: 433.2 [M + H]⁺. ¹H NMR (400 MHz, CDCl₃): δ 7.82 (br, 1H), 6.59 (br, 1H), 5.66 (br, 1H), 4.28~4.47 (m, 2H), 3.97 (d, J = 10.4 Hz, 2 H), 3.42~3.63 (m, 4 H), 2.86 (d, J = 13.6 Hz, 2H) 2.31 (s, 3 H) 2.06~2.21 (m, 2 H) 1.93 (d, J = 4.0 Hz, 2 H). |
| 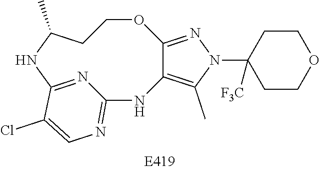 [SMILES: Cc1c2c(nn1C1(C(F)(F)F)CCOCC1)OCC[C@@H](C)Nc1nc(ncc1Cl)N2] | (11R)-14-Chloro-4,11-dimethyl-5-[4-(trifluoromethyl)oxan-4-yl]- 8-oxa-2,5,6,12,16,17-hexaazatricyclo[11.3.1.0^{3,7}]heptadeca- 1(16),3,6,13(17),14-pentaene (E419) Compound was prepared using procedures similar to those used for preparing E418. LC-MS: 447.3 [M + H]⁺. ¹H NMR (400 MHz, CDCl₃): δ 7.80 (s, 1H), 7.00 (br, 1H), 5.35 (d, J = 6.4 Hz, 1H), 4.38~4.55 (m, 1H), 4.15~4.32 (m, 1H), 3.87~4.07 (m, 3H), 3.37~3.63 (m, 2H), 2.86 (t, J = 12.4 Hz, 2H), 2.32 (s, 3H), 2.06~2.22 (m, 2H), 1.76~1.95 (m, 2H), 1.20~1.55 (m, 3H). |
| 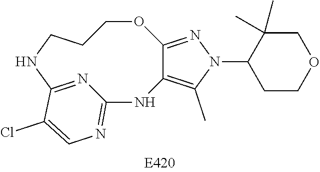 [SMILES: Cc1c2c(nn1C1CCOCC1(C)C)OCCCNc1nc(ncc1Cl)N2] | 14-Chloro-5-(3,3-dimethyloxan-4-yl)-4-methyl-8-oxa- 2,5,6,12,16,17-hexaazatricyclo[11.3.1.0^{3,7}]heptadeca- 1(16),3,6,13(17),14-pentaene (E420) Compound was prepared using procedures similar to those used for preparing E1, starting from D194. LC-MS: 393.2 [M + H]⁺. ¹H NMR (400 MHz, CDCl₃): δ 7.83 (s, 1H), 5.63 (br, 1H)., 4.41~4.32 (m, 2H), 4.20 (d, J = 15.2 Hz, 1H), 3.83 (d, J = 16.8 Hz, 1H), 3.64 (d, J = 11.2 Hz, 1H), 3.64~3.61 (m, 3H), 3.23 (d, J = 12.0 Hz, 1H), 2.63~2.53 (m, 1H), 2.22 (s, 3H), 2.01 (br, 1H), 1.89 (br, 2H), 1.25 (br, 1H), 1.10 (s, 3H), 0.82 (s, 3H). |
| 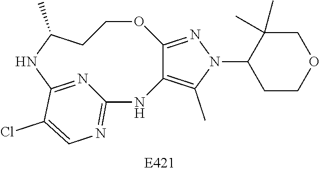 [SMILES: Cc1c2c(nn1C1CCOCC1(C)C)OCC[C@@H](C)Nc1nc(ncc1Cl)N2] | (11R)-14-Chloro-5-(3,3-dimethyloxan-4-yl)-4,11-dimethyl-8-oxa- 2,5,6,12,16,17-hexaazatricyclo[11.3.1.0^{3,7}]heptadeca- 1(16),3,6,13(17),14-pentaene (E421) Compound was prepared using procedures similar to those used for preparing E420. LC-MS: 407.2 [M + H]⁺. ¹H NMR (400 MHz, CDCl₃): δ 7.83 (s, 1H), 5.20 (br, 1H)., 4.51~4.47 (m, 1H), 4.29 (t, J = 10.8 Hz, 1H), 4.18 (d, J = 8.0 Hz, 1H), 3.99 (br, 1H), 3.63 (br, 1H), 3.61 (d, J = 12.0 Hz, 1H), 3.54 (t, J = 12.0 Hz, 1H), 3.24 (d, J = 12.0 Hz, 1H), 2.62~2.52 (m, 1H), 2.21 (s, 3H), 2.00~1.79 (m, 3H), 1.34~1.26 (m, 4H), 1.14 (s, 3H), 0.83 (s ,3H). |
| 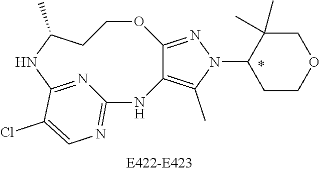 [SMILES: Cc1c2c(nn1C1CCOCC1(C)C)OCC[C@@H](C)Nc1nc(ncc1Cl)N2] | (11R)-14-Chloro-5-(3,3-dimethyloxan-4-yl)-4,11-dimethyl-8-oxa- 2,5,6,12,16,17-hexaazatricyclo[11.3.1.0^{3,7}]heptadeca- 1(16),3,6,13(17),14-pentaene (E422-E423) The title compounds E422 and E423 were obtained from chiral separation of E421 (chiral method A). E422: LC-MS: 407.2 [M + H]⁺. ¹H NMR (400 MHz, CDCl₃): δ 7.81 (s, 1H), 5.99 (s, 1H), 5.12 (d, J = 7.2 Hz, 1H), 4.50~4.47 (m, 1H), 4.32 (d, J = 10.4 Hz, 1H), 4.25 (d, J = 8.0 Hz, 1H), 4.16 (s, 1H), 3.81~3.77 (m, 1H), 3.64 (d, J = 12.0 Hz, 1H), 3.53 (d, J = 4.0 Hz, 1H), 3.24 (d, J = 12.0 Hz, 1H), 2.62 (s, 1H), 2.19 (s, 3H), 1.33 (t, J = 8.0 Hz, 3H), 1.28 (d, J = 12.0 Hz, 3H), 1.14 (s, 3H), 0.88 (s, 3H). Chiral RT = |
| | 14.516 min; ee > 99%. |
| | E423: LC-MS: 407.2 [M + H]⁺. ¹H NMR (400 MHz, CD₃OD): δ7.82 (s, |
| | 1H), 6.02 (s, 1H), 5.14 (d, J = 8.0 Hz, 1H), 4.60~4.42 (m, 1H), 4.34 |
| | (t, J = 20.0 Hz, 1H), 4.20 (d, J = 12.0 Hz, 1H), 4.00 (s, 1H), |
| | 3.83~3.79 (m, J = 16.0 Hz, 1H), 3.63 (d, J = 12.0 Hz, 1H), 3.57~3.51 |
| | (m, 1H), 3.22 (d, J = 12.0 Hz, 1H), 2.62(d, J = 4 Hz, 1H), 2.19 (s, |
| | 3H), 1.33 (t, J = 12.0 Hz, 3H), 1.28 (d, J = 12.0 Hz, 3H), 1.06 (s, 3H), |
| | 0.81 (s, 3H). |
| 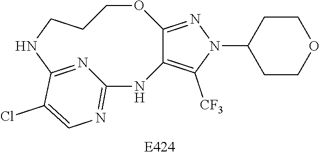 [SMILES: FC(F)(F)c1c2c(nn1C1CCOCC1)OCCCNc1nc(ncc1Cl)N2] | 14-Chloro-5-(oxan-4-yl)-4-(trifluoromethyl)-8-oxa-2,5,6,12,16,17- hexaazatricyclo[11.3.1.0^{3,7}]heptadeca-1(16),3,6,13(17),14- pentaene (E424) Compound was prepared using procedures similar to those used for preparing E416. LC-MS: 419.3 [M + H]⁺. ¹H NMR (400 MHz, CD₃OD): δ 7.75 (s, 1H), 4.38 (t, J = 4.0 Hz, 2H), 4.36~4.30 (m, 1H), 4.06 (dd, J = 12.0 Hz, 2H), 3.57~3.51 (m, 2H), 3.42 (t, J = 4.0 Hz, 2H), 2.28~2.19 (m, 2H), 1.87~1.84 (m, 4H). |
| 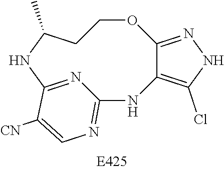 [SMILES: C[C@@H]1CCOc2n[nH]c(Cl)c2Nc2ncc(C#N)c(n2)N1] | (11R)-4-Chloro-11-methyl-8-oxa-2,5,6,12,16,17- hexaazatricyclo[11.3.1.0^{3,7}]heptadeca-1(16),3,6,13(17),14- pentaene-14-carbonitrile (E425) Compound was prepared using procedures similar to those used for preparing E88, starting from 3,5-dichloro-4-nitro-1-(tetrahydro-2H- pyran-2-yl)-1H-pyrazole. LC-MS: 306.2 [M + H]⁺. ¹H NMR (400 MHz, DMSO-d₆): δ 12.85~12.60 (br, 1H), 9.21~9.02 (br, 1H), 8.26 (s, 1H), 7.69-7.67 (br, 1H), 4.46~3.91 (m, 3H), 1.79~1.67 (m, 2H), 1.23 (s, 3H). |
| 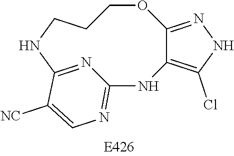 [SMILES: N#Cc1cnc2nc1NCCCOc1n[nH]c(Cl)c1N2] | 4-Chloro-8-oxa-2,5,6,12,16,17- hexaazatricyclo[11.3.1.0^{3,7}]heptadeca-1(16),3,6,13(17),14- pentaene-14-carbonitrile (E426) Compound was prepared using procedures similar to those used for preparing E425. LC-MS: 292.3 [M + H]⁺. ¹H NMR (400 MHz, DMSO-d₆): δ 12.76 (br, 1H), 9.09 (br, 1H), 8.25 (s, 1H), 7.99~7.97 (br, 1H), 4.28~4.21 (m, 2H), 2.67 (t, J = 1.6 Hz, 2H), 1.78 (s, 2H). |
| 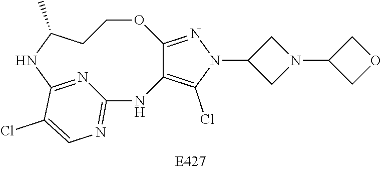 [SMILES: C[C@@H]1CCOc2nn(C3CN(C4COC4)C3)c(Cl)c2Nc2ncc(Cl)c(n2)N1] | (11R)-4,14-Dichloro-11-methyl-5-[1-(oxetan-3-yl)azetidin-3-yl]-8- oxa-2,5,6,12,16,17-hexaazatricyclo[11.3.1.0^{3,7}]heptadeca- 1(16),3,6,13(17),14-pentaene (E427) Compound was prepared using procedures similar to those used for preparing E144, starting from (R)-2-(4-hydroxybutan-2- yl)isoindoline-1,3-dione and 4-nitro-1-((2- (trimethylsilyl)ethoxy)methyl)-1H-pyrazole. LC-MS: 426.2 [M + H]⁺. ¹H NMR (400 MHz, CDCl₃): δ 7.85 (s, 1H), 6.29 (s, 1H), 5.15 (d, J = 6.4 Hz, 1H), 5.08~5.01 (m, 1H), 4.76 (t, J = 7.2 Hz, 2H), 4.69 (t, J = 6.0 Hz, 1H), 4.64~4.61 (m, 1H), 4.52~4.47 (m, 1H), 4.39~4.34 (m, 1H), 4.07~4.00 (m, 1H), 3.98~3.92 (m, 1H), |
| | 3.83~3.76 (m, 3H), 3.68 (t, J = 7.8 Hz, 1H), 1.97~1.83 (m, 1H), |
| | 1.35 (d, J = 6.8 Hz, 1H). |
| 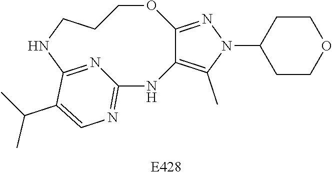 [SMILES: Cc1c2c(nn1C1CCOCC1)OCCCNc1nc(ncc1C(C)C)N2] | 4-Methyl-5-(oxan-4-yl)-14-(propan-2-yl)-8-oxa-2,5,6,12,16,17- hexaazatricyclo[11.3.1.0^{3,7}]heptadeca-1(16),3,6,13(17),14- pentaene (E428) Compound was prepared using procedures similar to those used for preparing E1, starting from 2,4-dichloro-5-isopropylpyrimidine. LC-MS: 373.3 [M + H]⁺. ¹H NMR (400 MHz, CD₃OD): δ 7.50 (s, 1H), 4.35 (t, J = 4.6 Hz, 2H), 4.32~4.25 (m, 1H), 4.07~4.02 (m, 2H), 3.57 (t, J = 11.4 Hz, 2H), 3.49 (t, J = 5.4 Hz, 2H), 2.81~2.77 (m, 2H), 2.26 (s, 3H), 2.21~2.12 (m, 2H), 1.90~1.75 (m, 4H), 1.22 (d, J = 6.8 Hz, 6H). |
| 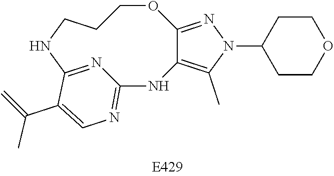 [SMILES: C=C(C)c1cnc2nc1NCCCOc1nn(C3CCOCC3)c(C)c1N2] | 4-Methyl-5-(oxan-4-yl)-14-(prop-1-en-2-yl)-8-oxa-2,5,6,12,16,17- hexaazatricyclo[11.3.1.0^{3,7}]heptadeca-1(16),3,6,13(17),14- pentaene (E429) Compound was prepared using procedures similar to those used for preparing E428. LC-MS: 371.2 [M + H]⁺. ¹H NMR (400 MHz, CD₃OD): δ 7.55 (s, 1H), 5.30 (s, 1H), 5.10 (s, 1H), 4.34 (t, J = 4.6 Hz, 2H), 4.30~4.22 (m, 1H), 4.07~4.00 (m, 2H), 3.57 (t, J = 11.8 Hz, 2H), 3.43 (t, J = 5.2 Hz, 2H), 2.26 (s, 3H), 2.18~2.10 (m, 2H), 2.01 (s, 3H), 1.85~1.77 (m, 4H). |
| 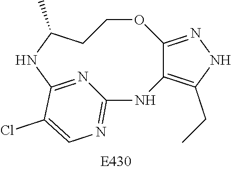 | (11R)-14-Chloro-4-ethyl-11-methyl-8-oxa-2,5,6,12,16,17- hexaazatricyclo[11.3.1.0^{3,7}]heptadeca-1(16),3,6,13(17),14- entaene (E430) Compound was prepared using procedures similar to those used for preparing E294, starting from 4-nitro-1-(tetrahydro-2H-pyran-2-yl)- 1H-pyrazole. LC-MS: 393.2 [M + H]⁺. ¹H NMR (400 MHz, MeOD): δ 7.73 (s, 1H), 4.41~4.26 (m, 2H), 4.00~3.96 (m, 1H), 2.68~2.54 (m, 2H), 1.81~1.78 (m, 2H), 1.29 (d, J = 8.0 Hz, 3H), 1.22 (t, J = 16.0 Hz, 3H). |
| 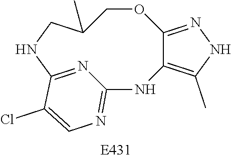 [SMILES: Cc1[nH]nc2c1Nc1ncc(Cl)c(n1)NCC(C)CO2] | 14-Chloro-4,10-dimethyl-8-oxa-2,5,6,12,16,17- hexaazatricyclo[11.3.1.0^{3,7}]heptadeca-1(16),3,6,13(17),14- pentaene (E431) Compound was prepared using procedures similar to those used for preparing E394, starting from 3-amino-2-methylpropanoic acid. LC-MS: 295.1 [M + H]⁺. ¹HNMR (400 MHz, CD₃OD): δ 7.71 (s, 1H), 4.57 (s, 1H), 4.19~4.09 (m, 2H), 3.68~3.63 (m, 1H), 3.06 (d, J = 12.0 Hz, 1H), 2.19 (s, 3H), 1.81 (br, 1H), 0.99 (d, J = 7.4 Hz, 3H). |
| 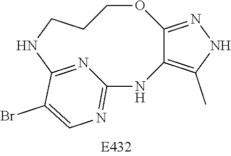 [SMILES: Cc1[nH]nc2c1Nc1ncc(Br)c(n1)NCCCO2] | 14-Bromo-4-methyl-8-oxa-2,5,6,12,16,17- hexaazatricyclo[11.3.1.0^{3,7}]heptadeca-1(16),3,6,13(17),14- pentaene (E432) Compound was prepared using procedures similar to those used for preparing E394, starting from 5-bromo-2,4-dichloropyrimidine. LC-MS: 326.1 [M + H]⁺. ¹H NMR (400 MHz, DMSO-d₆): δ 11.39 (s, 1H), 8.25 (s, 1H), 7.83 (s, 1H), 7.13 (t, J = 6.0 Hz, 1H), 4.18 (t, J = 4.4 Hz, 2H), 3.29~3.26 (m, 2H), 2.09 (s, 3H), 1.70 (s, 2H). |
| 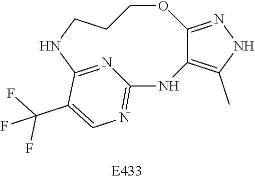 | 4-Methyl-14-(trifluoromethyl)-8-oxa-2,5,6,12,16,17- hexaazatricyclo[11.3.1.0^{3,7}]heptadeca-1(16),3,6,13(17),14- pentaene (E433) Compound was prepared using procedures similar to those used for preparing E394. LC-MS: 329.0 [M + H]⁺. ¹H NMR (400 MHz, CD₃OD): δ 8.03 (s, 1H), 4.36~4.34 (m, 2H), 3.82~3.78 (m, 1H), 2.20 (s, 3H), 1.80~1.77 (m, 2H), 1.23 (d, J = 6.8 Hz, 3H). |
| 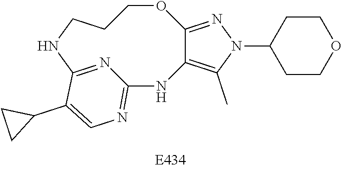 [SMILES: Cc1c2c(nn1C1CCOCC1)OCCCNc1nc(ncc1C1CC1)N2] | 14-Cyclopropyl-4-methyl-5-(oxan-4-yl)-8-oxa-2,5,6,12,16,17- hexaazatricyclo[11.3.1.0^{3,7}]heptadeca-1(16),3,6,13(17),14- pentaene (E434) Compound was prepared using procedures similar to those used for preparing E1, starting from 2,4-dichloro-5-iodopyrimidine. LC-MS: 371.3 [M + H]⁺. ¹H NMR (400 MHz, MeOD): δ 7.46 (s, 1H), 4.34 (t, J = 12.0 Hz, 2H)., 4.28~4.22(m, 1H), 4.05 (dd, J = 16.0 Hz, 2H), 3.56 (t, J = 24.0 Hz, 2H), 3.45 (t, J =12.0 Hz, 2H), 2.23 (s, 1H), 2.17 (dd, J = 16.0 Hz, 2H), 1.93~1.76 (m, 4H), 1.47~1.44 (m, 1H), 0.84 (dd, J = 8.0 Hz, 2H), 0.45 (dd, J = 8.0 Hz, 2H) |
| 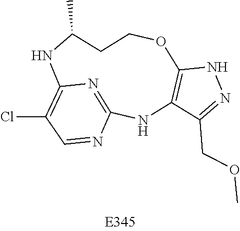 [SMILES: COCc1n[nH]c2c1Nc1ncc(Cl)c(n1)N[C@H](C)CCO2] | (11R)-14-Chloro-4-(methoxymethyl)-11-methyl-8-oxa- 2,5,6,12,16,17-hexaazatricyclo[11.3.1.0^{3,7}]heptadeca- 1(17),3(7),4,13,15-pentaene (E435) Compound was prepared using procedures similar to those used for preparing E424. LC-MS: 325.3 [M + H]⁺. ¹H NMR (400 MHz, MeOD): δ 7.74 (s, 1H), 4.42~4.40 (m, 3H), 4.31~4.26 (m, 1H), 3.98 (s, 1H), 3.35~3.30 (m, 3H), 1.85~1.82 (m, 2H), 1.31~1.26 (d, J = 20.0 Hz, 3H). |
| 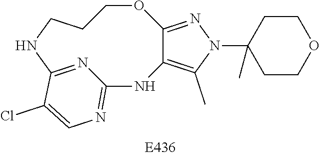 [SMILES: Cc1c2c(nn1C1(C)CCOCC1)OCCCNc1nc(ncc1Cl)N2] | 14-Chloro-4-methyl-5-(4-methyloxan-4-yl)-8-oxa-2,5,6,12,16,17- hexaazatricyclo[11.3.1.0^{3,7}]heptadeca-1(16),3,6,13(17),14- pentaene (E436) E436 Compound was prepared using procedures similar to those used for preparing E420, starting from 2-(2-methyl-1,3-dioxolan-2- yl)acetohydrazide and dihydro-2H-pyran-4(3H)-one. LC-MS: 379.2 [M + H]⁺. ¹H NMR (400 MHz, MeOD): δ 7.71 (s, 1H), 4.32 (t, J = 8.0 Hz, 2H), 3.78~3.68 (m, 4H), 3.41 (t, J = 8.0 Hz, 2H), 2.57~2.53 (m, 2H) 2.31 (s, 3H), 1.95~1.81 (m, 4 H), 1.44 (s, 3H). |
| 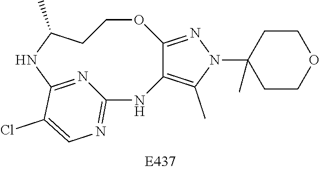 [SMILES: Cc1c2c(nn1C1(C)CCOCC1)OCC[C@@H](C)Nc1nc(ncc1Cl)N2] | (11R)-14-Chloro-4,11-dimethyl-5-(4-methyloxan-4-yl)-8-oxa- 2,5,6,12,16,17-hexaazatricyclo[11.3.1.0^{3,7}]heptadeca- 1(16),3,6,13(17),14-pentaene (E437) Compound was prepared using procedures similar to those used for preparing E436. LC-MS: 393.2 [M + H]⁺. ¹H NMR (400 MHz, MeOD): δ 7.73 (s, 1H), 4.35~4.32 (m, 2H), 3.97~3.93 (m, 1H), 3.76~3.69 (m, 4H), 2.55 (dd, J = 16.0 Hz, 2H), 2.23 (s, 3H), 1.95~1.89 (m, 2 H), 1.80~1.76 (m, 2 H), 1.44 (s, 3H), 1.30 ( t, J = 8.0 Hz, 3H). |
| 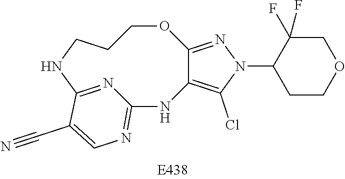 [SMILES: N#Cc1cnc2nc1NCCCOc1nn(C3CCOCC3(F)F)c(Cl)c1N2] | 4-Chloro-5-(3,3-difluorooxan-4-yl)-8-oxa-2,5,6,12,16,17- hexaazatricyclo[11.3.1.0^{3,7}]heptadeca-1(16),3,6,13(17),14- pentaene-14-carbonitrile (E438) Compound was prepared using procedures similar to those used for preparing E414, starting from D195 and 4-nitro-1H-pyrazole. LC-MS: 412.3 [M + H]⁺. ¹H NMR (400 MHz, CDCl₃): δ 8.17 (s, 1H), 7.79 (s, 1H), 5.97 (s, 1H), 4.71~4.52 (m, 1H), 4.47~4.31 (m, 2H), 4.31~4.25 (m, 1H), 4.10 (dd, J = 21.2, 9.1 Hz, 1H), 3.95 (t, J = 5.4 Hz, 1H), 3.70~3.65 (m, 1H), 3.62~3.55 (m, 2H), 2.78~2.58 (m, 1H), 2.18~2.10 (m, 1H), 1.98 (s, 2H). |
| 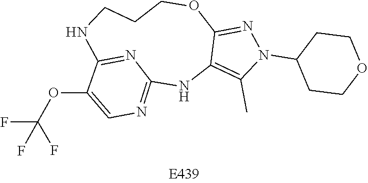 [SMILES: Cc1c2c(nn1C1CCOCC1)OCCCNc1nc(ncc1OC(F)(F)F)N2] | 4-Methyl-5-(oxan-4-yl)-14-(trifluoromethoxy)-8-oxa- 2,5,6,12,16,17-hexaazatricyclo[11.3.1.0^{3,7}]heptadeca- 1(16),3,6,13(17),14-pentaene (E439) Compound was prepared using procedures similar to those used for preparing E429, starting from 2,4-dichloropyrimidin-5-ol. LC-MS: 415.1 [M + H]⁺. ¹H NMR (400 MHz, CDCl₃): δ 9.40 (s, 1H), 7.60 (s, 1H), 6.20 (s, 1H), 4.30~4.28 (m, 3H), 4.05~4.01 (m, 2H), 3.58~3.52 (m, 2H), 3.43 (t, J = 12.0 Hz, 2H), 2.17 (s, 3H), 2.22~2.13 (m, 2H), 1.97~1.91 (m, 2H), 1.71 (d, J = 10.8 Hz, 2H). |
| 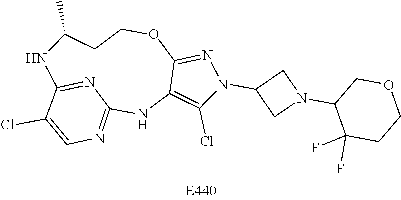 [SMILES: C[C@@H]1CCOc2nn(C3CN(C4COCCC4(F)F)C3)c(Cl)c2Nc2ncc(Cl)c(n2)N1] | (11R)-4,14-Dichloro-5-[1-(4,4-difluorooxan-3-yl)azetidin-3-yl]-11- methyl-8-oxa-2,5,6,12,16,17-hexaazatricyclo[11.3.1.0^{3,7}]heptadeca- 1(16),3,6,13(17),14-pentaene, TFA salt (E440) Compound was prepared using procedures similar to those used for preparing E439, starting from D195 and 4-nitro-1H-pyrazole. LC-MS: 490.1 [M + H]⁺. ¹H NMR (400 MHz, CDCl₃): δ 7.64 (s, 1H), 6.07 (t, J = 7.2 Hz, 1H), 5.3~5.29 (m, 1H), 4.60 (t, J = 7.2 Hz, 1H), 4.51~4.45 (m, 2H), 4.33~4.14 (m, 2H), 4.03~3.96 (m, 2H), 3.81~3.71 (m, 4H), 3.40~3.37 (m, 1H), 2.40 (br, 1H), 2.05 (br, 1H), 1.90 (s, 2H), 1.37 (d, J = 6.8 Hz, 3H). |
| 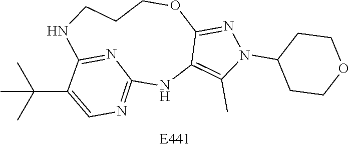 [SMILES: Cc1c2c(nn1C1CCOCC1)OCCCNc1nc(ncc1C(C)(C)C)N2] | 14-tert-Butyl-4-methyl-5-(oxan-4-yl)-8-oxa-2,5,6,12,16,17- hexaazatricyclo[11.3.1.0^{3,7}]heptadeca-1(16),3,6,13(17),14- pentaene (E441) Compound was prepared using procedures similar to those used for preparing E1, starting from D199. LC-MS: 387.3 [M + H]⁺. ¹H NMR (400 MHz, CDCl₃): δ 7.77 (s, 1H), 6.11 (br, 1H), 5.14 (t, J = 5.6 Hz, 1H), 4.42 (t, J = 4.8 Hz, 2H), 4.12~4.03 (m, 3H), 3.56~3.47 (m, 4H), 2.32~2.25 (m, 2H), 2.23 (s, 3H), 1.91~1.86 (m, 2H), 1.79~1.71 (m, 2H), 1.30 (s, 9H). |
| 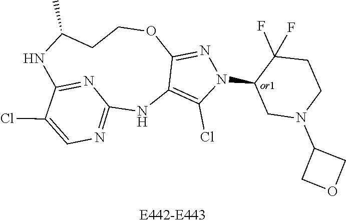 [SMILES: C[C@@H]1CCOc2nn([C@@H]3CN(C4COC4)CCC3(F)F)c(Cl)c2Nc2ncc(Cl)c(n2)N1] | Enantiomers1-2: (11R)-4,14-Dichloro-5-[4,4-difluoro-1-(oxetan- 3-yl)piperidin-3-yl]-11-methyl-8-oxa-2,5,6,12,16,17- hexaazatricyclo[11.3.1.0^{3,7}]heptadeca-1(16),3,6,13(17),14- pentaene (E442-E443) Compound was prepared using procedures similar to those used for preparing E411. E442: LC-MS: 490.3 [M + H]⁺. ¹H NMR (400 MHz, MeOD): δ 7.76 (s, 1H), 4.78~4.69 (m, 4H), 4.64~4.60 (m, 2H), 4.40~4.33 (m, 2H), 3.93~3.91 (m, 1H), 3.72 (t, J = 16.0 Hz, 1H), 3.07~3.04 (m, 1H), 2.96~2.86 (m, 2H), 2.23~2.22 (m, 2H), 1.80~1.77 (m, 2H), 1.29 (d, J = 8.0 Hz, 3H). E442: LC-MS: 490.3 [M + H]⁺. ¹H NMR (400 MHz, MeOD): δ 7.76 (s, 1H), 4.73~4.70 (m, 3H), 4.69~4.60 (m, 2H), 4.59~4.54 (m, 1H), 4.38~4.34 (m, 2H), 3.99~3.96 (m, 1H), 3.30 (t, J = 16.0 Hz, 1H), 2.93~2.86 (m, 3H), 2.25-2.17 (m, 2H), 1.81~1.79 (m, 2H), 1.29(d, |
| | J = 8.0 Hz, 3H). |
| 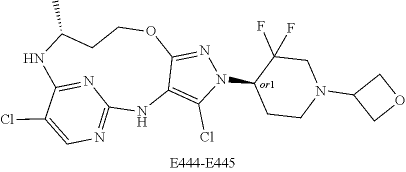 [SMILES: C[C@@H]1CCOc2nn([C@@H]3CCN(C4COC4)CC3(F)F)c(Cl)c2Nc2ncc(Cl)c(n2)N1] | Enantiomers1-2: (11R)-4,14-Dichloro-5-[3,3-difluoro-1-(oxetan- 3-yl)piperidin-4-yl]-11-methyl-8-oxa-2,5,6,12,16,17- hexaazatricyclo[11.3.1.0^{3,7}]heptadeca-1(16),3,6,13(17),14- pentaene (E444-E445) Compound was prepared using procedures similar to those used for preparing E411. E444: LC-MS: 490.2 [M + H]⁺. ¹H NMR (400 MHz, MeOD): δ 7.76 (s, 1H), 4.78~4.58 (m, 5H), 4.39~4.37 (m, 2H), 3.95~3.91 (m, 1H), 3.73 (t, J = 16.0 Hz, 1H), 3.09~3.00 (m, 2H), 2.67~2.64 (m, 1H), 2.53~2.46 (m, 1 H), 2.32 (t, J = 16.0 Hz, 1H), 2.09~2.06 (m, 1 H), 1.83~1.78 (m, 2 H), 1.30 (d, J = 8.0 Hz, 3H). E445: LC-MS: 490.2 [M + H]⁺. ¹H NMR (400 MHz, MeOD): δ 7.76 (s, 1H), 4.72~4.58 (m, 5H), 4.42~4.37 (m, 2H), 4.00~3.98 (m, 1H), 3.73 (t, J = 18.0 Hz, 1H), 3.13~3.01 (m, 2H), 2.84~2.45 (m, 2H), 2.32 (t, J = 16.0 Hz, 1H), 2.02~1.99 (m, 1 H), 1.83~1.80 (m, 2 H), 1.30 (d, J= 8.0 Hz, 3H). |
| 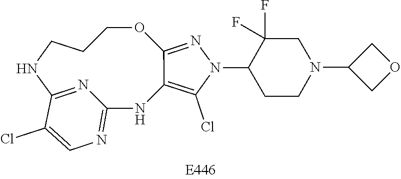 [SMILES: FC1(F)CN(C2COC2)CCC1n1nc2c(c1Cl)Nc1ncc(Cl)c(n1)NCCCO2] | 4,14-Dichloro-5[3,3-difluoro-1-(oxetan-3-yl)piperidin-4-yl]-8- oxa-2,5,6,12,16,17-hexaazatricyclo[11.3.1.0^{3,7}]heptadeca- 1(16),3,6,13(17),14-pentaene (E446) E446 Compound was prepared using procedures similar to those used for preparing E411. LC-MS: 476.3 [M + H]⁺. ¹H NMR (400 MHz, DMSO-d₆): δ 8.61 (s, 1H), 7.82 (s, 1H), 7.46 (t, J = 5.6 Hz, 1H), 4.61~4.44 (m, 5H), 4.26~4.19 (m, 2H), 3.65 (t, J = 6.2 Hz, 1H), 3.05-2.98 (m, 1H), 2.84-2.75 (m, 2H), 2.21~2.09 (m, 3H), 1.84~1.75 (m, 1H), 1.72~1.61 (m, 1H), 1.35~1.20 (m, 2H). |
| 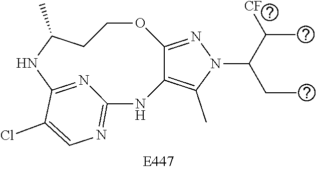 | (11R)-14-Chloro-4,11-dimethyl-5-[3-(trifluoromethyl)oxan-4-yl]- 8-oxa-2,5,6,12,16,17-hexaazatricyclo[11.3.1.0^{3,7}]heptadeca- 1(16),3,6,13(17),14-pentaene (E448) Compound was prepared using procedures similar to those used for preparing E1, starting from D203. LC-MS: 447.1 [M + H]⁺. ¹H NMR (400 MHz, CDCl₃): δ 7.82 (d, J = 4.0 Hz, 1H), 6.06 (d, J = 20.0 Hz, 1H)., 5.11 (d, J = 8.0 Hz, 1H), 4.67~4.57 (m, 3H), 4.46 (d, J = 8.0 Hz, 1H), 4.37~4.31 (m, 1H), 3.96 (d, J = 16.0 Hz, 2H), 3.84 (m, 1H), 2.73 (br, 1H), 2.18 (t, J = 4.0 Hz, 5H), 1.87~1.80 (m, 1H), 1.32~1.26 (m, 4H). |
| 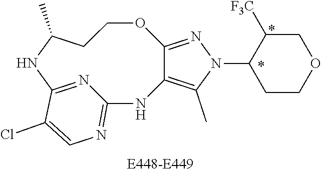 [SMILES: Cc1c2c(nn1C1CCOCC1C(F)(F)F)OCC[C@@H](C)Nc1nc(ncc1Cl)N2] | Isomers1-2: (11R)-14-Chloro-4,11-dimethyl-5[3- (trifluoromethyl)oxan-4-yl]-8-oxa-2,5,6,12,16,17- hexaazatricyclo[11.3.1.0^{3,7}]heptadeca-1(16),3,6,13(17),14- pentaene (E448-E449) The title compounds E448 and E449 were obtained from chiral separation of E447 (chiral method E). E448: LC-MS: 447.1 [M + H]⁺. ¹H NMR (400 MHz, CD₃OD): δ 7.72(s, 1H), 4.51 (t, J = 16.0 Hz, 2H), 4.37 (d, J = 12.0 Hz, 2H), 3.89 (br, 1H), 3.82~3.74 (m, 1H), 2.89 (br, 1H), 2.16 (s, 3H), 2.04~2.01 (m, 2H), 1.79 (br, 2H), 1.63~1.52 (m, 1H), 1.29 (s, 3H), 0.90~0.88 (m, 1H). Chiral RT = 0.845 min (chiral method E); ee > 99%. |
| | E449: LC-MS: 447.2 [M + H]⁺. ¹H NMR (400 MHz, CD₃OD): δ 7.71 |
| | (s, 1H), 4.82 (s, 1H), 4.73~4.49 (m, 1H), 4.49~4.36 (m, 3H), |
| | 3.94~3.93 (m, 1H), 3.81 (dd, J = 11.6 Hz, 3.6 Hz, 1H), 3.72~3.69 (m, |
| | 1H), 2.84-2.83 (m, 1H), 2.25-2.23 (m, 1H), 2.20 (s, 3H), 2.04-2.00 |
| | (m, 1H), 1.77~1.76 (m, 2H), 1.26 (d, J = 7.2 Hz, 3H). Chiral RT = |
| | 1.078 min (chiral method E); ee > 99%. |
| 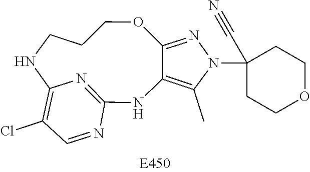 [SMILES: Cc1c2c(nn1C1(C#N)CCOCC1)OCCCNc1nc(ncc1Cl)N2] | 4-{14-chloro-4-methyl-8-oxa-2,5,6,12,16,17- hexaazatricyclo[11.3.1.0^{3,7}]heptadeca-1(17),3,6,13,15-pentaen-5- yl}oxane-4-carbonitrile (E450) Compound was prepared using procedures similar to those used for preparing E436, starting from TMSCN. LC-MS: 390.1 [M + H]⁺. ¹H NMR(400 MHz, CDCl₃): δ 7.81 (s, 1 H), 6.63 (s, 1 H ), 5.66~5.67 (m, 1 H), 4.35~4.37 (m, 2 H), 4.07~4.10 (t, J = 12.0 Hz, 2 H), 3.80~3.86 (br, 2 H), 3.51~3.55 ( m, 2 H), 2.46~2.54 (m, 5 H), 2.36~2.39 (t, J = 12.0 Hz, 2 H), 1.75~1.91 (t, J = 64.0 Hz, 2 H). |
| 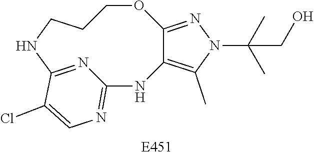 [SMILES: Cc1c2c(nn1C(C)(C)CO)OCCCNc1nc(ncc1Cl)N2] | 2-{14-Chloro-4-methyl-8-oxa-2,5,6,12,16,17-hexaazatricyclo[11. 3.1.0^{3,7}]heptadecal 1(16),3,6,13(17),14-pentaen-5-yl}-2-methylpropan- 1-ol (E451) A solution of E188 (100 mg, 0.25 mmol) in THF (20 mL) was added NaBH₄ (28 mg, 0.75 mmol) and LiCl (32 mg, 0.75 mmol) at rt. The mixture was stirred at rt overnight. The reaction was quenched with water and the mixture was extracted with EtOAc (50 mL × 3). The combined organic solution was washed with brine (50 mL × 2), dried, filtered and concentrated. The residue was purified by prep-HPLC to afford the desired product (4 mg, yield 4%) as a light red solid. LC-MS: 353.3 [M + H]⁺. ¹H NMR (400 MHz, CDCl₃): δ 7.82 (s, 1H), |
| | 6.05 (s, 1H), 5.55 (m, 1H), 4.54 (m, 1H), 4.33~4.35 (m, 2H), 3.84~3.86 |
| | (m, 2H), 3.49~3.54 (m, 2H), 2.33 (s, 3H), 1.90~1.93 (m, 2H), 1.50 (s, |
| | 6H). |
| 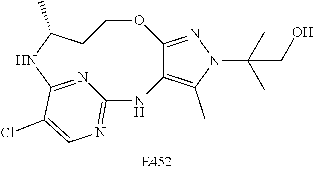 [SMILES: Cc1c2c(nn1C(C)(C)CO)OCC[C@@H](C)Nc1nc(ncc1Cl)N2] | 2-[(11R)-14-Chloro-4,11-dimethyl-8-oxa-2,5,6,12,16,17- hexaaza tricyclo[11.3.1.0^{3,7}]heptadeca-1(16),3,6,13(17),14-pentaen-5-yl]- 2-methylpropan-1-ol (E452) E452 Compound was prepared using procedures similar to those used for preparing E451, starting from D79. LC-MS: 367.2 [M + H]⁺. ¹H NMR (400 MHz, CDCl₃): δ 7.81 (s, 1H), 6.32 (s, 1H), 5.15~5.13 (m, 1H), 4.59 (brs, 1H), 4.46~4.24 (m, 2H), 4.02~4.00 (m, 1H), 3.90~3.81 (m, 2H), 2.32 (s, 3H), 1.89~1.77 (m, 2H), 1.52 (s, 3H), 1.47 (s, 3H), 1.33~1.31 (m, 3H). |

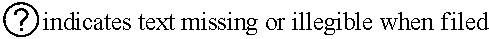

The compounds of descriptions D84, D93, D97, D100, D112, D130, D133, D142, D160 and D162 are also examples of compounds falling within the scope of the invention.

The compound of Example 6 may also be prepared using the following procedure. This procedure has been followed to produce a total of 485.8 g compound in several batches. (single batch of compound produced from multiple batches of intermediates):

### Preparation of 5-chloro-3-methyl-4-nitro-1-(tetrahydro-2H-pyran-2-yl)-1H-pyrazole

3-Methyl-4-nitro-1H-pyrazole (500 g) and p-toluenesulfonic acid monohydrate (15.0 g) were dissolved in 2-methyltetrahydrofuran (2.5 L) at c. 27° C. and stirred for 15 mins. The solution was then cooled to c.−11° C. and treated with 3,4-dihydro-2H-pyran (430 mL). The reaction mixture was stirred for c. 22 h at c.−11° C. before being cooled to c.−63° C. and treated with a 1.5 M solution of LiHMDS in THF (2.9 L) over c.60 min. The reaction mixture was stirred at −63° C. for 45 min before a solution of perchloroethane (1024 g) in 2-methyl THF (2 L) was added over 80 min. The reaction was stirred for a further 50 min at c. −63° C., warmed to 18° C. and stirred for a further 15 min before being quenched with a solution of ammonium chloride (930 g) in water (1.57 L). The pH was adjusted to 5-6 by addition of 2 M HCl (2.5 L) followed by acetic acid (19 mL) before TBME (3 L) was added and the mixture was stirred at c.18° C. for 16 mins. The mixture was filtered through celite (100 g) and washed through with further TBME (1 L). The aqueous layer was separated and back extracted with TBME (1 L). The combined organic phases were then washed twice with 1 L of a 1:1 mixture of saturated aqueous sodium bicarbonate and saturated aqueous sodium chloride. The washed organic phase was concentrated to 2 L. by vacuum distillation and the temperature was adjusted to c. 42° C. before heptane (6.5 L) was added over 25 min. The slurry was stirred at c.41° C. for 15 min, cooled to 2° C. over 2 h and then stirred at 2° C. for 1 h before the product was collected by filtration. The product cake was washed twice with 1 L of a 4:1 mixture of TBME and heptane and dried under vacuum at c. 30° C. to deliver the desired product 5-chloro-3-methyl-4-nitro-1-(tetrahydro-2H-pyran-2-yl)-1H-pyrazole (549 g, 56.8%).

### Preparation of (R)-4-((3-methyl-4-nitro-1H-pyrazol-5-yl)oxy)butan-2-amine hydrochloride

To a mixture of powdered cesium fluoride (1.85 kg) and tert-butyl (R)-(4-hydroxybutan-2-yl)carbamate (635 g) in N,N-dimethylacetamide (DMA) (3.75 L) was added 5-chloro-3-methyl-4-nitro-1-(tetrahydro-2H-pyran-2-yl)-1H-pyrazole (750 g) and the slurry was heated to 50° C. for c. 22 h. After cooling the reaction mixture to 20° C. water (3.75 L) and TBME (3.75 L) were added, the mixture was stirred for 10 min and the aqueous phase was separated. The organic phase was then washed twice with saturated aqueous ammonium chloride solution (2×3.75 L) followed by 15% w/w aqueous NaCl solution (3.75 L). The washed organic phase was concentrated to 2.25 L by vacuum distillation before IPA (3.75 L) was added and the mixture further concentrated to 3.75 L by vacuum distillation. After clarification of the concentrated solution by filtration, using IPA (0.75 L) as a line wash, a 5-6 M solution of HCl in IPA (3.1 L) was added and the reaction heated to 50° C. and stirred for c. 1 h. The resulting slurry was cooled to c. 22° C. before TBME (6.0 L) was added. The slurry was then aged at c. 22° C. for 1 h before the product was collected by filtration and the cake washed with 2:1 v/v TBME/IPA (2.25 L) followed by TBME (1.5 L). The damp cake was dried under vacuum at 30° C. to deliver the desired product (R)-4-((3-methyl-4-nitro-1H-pyrazol-5-yl)oxy)butan-2-amine hydrochloride (599 g, 78.3%)

### Preparation of (R)-2,5-dichloro-N-(4-((5-methyl-4-nitro-1-(tetrahydro-2H-pyran-4-yl)-1H-pyrazol-3-yl)oxy)butan-2-yl)pyrimidin-4-amine

A mixture of (R)-4-((3-methyl-4-nitro-1H-pyrazol-5-yl)oxy)butan-2-amine hydrochloride (700 g) and triethylamine (1.23 L) in 2-methyltetrahydrofuran (7.0 L) was warmed to c. 58° C. for c. 30 min and then treated with 2,4,5-trichloropyrimidine (320 mL) over 3 h. The reaction was stirred at 58° C. for c. 3 h before the slurry was cooled to c. 20° C. and washed with water (3.5 L) followed by 20% w/w aqueous ammonium chloride solution (3.5 L) and then 15% w/w aqueous NaCl solution (3.5 L). The washed organic layer was concentrated to 2.1 L by vacuum distillation before 2-methyl THF (3.5 L) was added and the mixture again concentrated to 2.1 L by vacuum distillation.

DMSO (3.0 L) was added and the solution was concentrated to 4.5 vol by vacuum distillation before additional DMSO (2.6 L) was added. Tetrahydro-2H-pyran-4-yl methanesulfonate (1.26 kg) and caesium carbonate (1.80 kg) were added and the reaction was heated to c. 80° C. for c.17 h. Upon reaction completion the mixture was cooled to 20° C. before water (5.0 L) and isopropyl acetate (5.0 L) were added. The aqueous layer was back extracted with 2:1 v/v isopropyl acetate/heptane (6.0 L). Water (3.0 L) was added to the combined organic layers with vigorous stirring for 10 min and the obtained biphasic solution was decanted from the precipitated red interfacial gum. The aqueous phase was disgarded and the organic phase was further washed with 10% aq NaCl solution. The washed organic phase was concentrated to dryness under vacuum before being re-dissolved in ethanol (3.0 L) at c. 62° C., Water (1.0 L) was added and the mixture cooled to 18° C. over c. 4 h. The obtained slurry was stirred at c. 18° C. for a further c.15 h before the product was collected by filtration and washed twice with 3:1 v/v EtOH/water. The damp cake was dried under vacuum at 40° C. to deliver the desired product (R)-2,5-dichloro-N-(4-((5-methyl-4-nitro-1-(tetrahydro-2H-pyran-4-yl)-1H-pyrazol-3-yl)oxy)butan-2-yl)pyrimidin-4-amine (572 g, 45.2%)

### Preparation of intermediate grade (11R)-14-chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene

A mixture of (R)-2,5-dichloro-N-(4-((5-methyl-4-nitro-1-(tetrahydro-2H-pyran-4-yl)-1H-pyrazol-3-yl)oxy)butan-2-yl)pyrimidin-4-amine (398.7 g), methanol (4 L), 1% Pd/2% V on carbon, 61% wet (160.0 g), and 50% aq phosphinic acid (23.6 g) was hydrogenated under c. 3 bar hydrogen pressure at c. 50° C. for c. 16 h until reaction complete was observed by HPLC. The reaction mixture was filtered to remove the catalyst, the catalyst cake was washed with methanol (0.8 L) and the filtrate was solvent exchanged with 2-methyltetrahydrofuran. The 2-methyltetrahydrofuran solution (2.4 L) was heated to 57° C. and washed with 6% w/w aqueous sodium bicarbonate solution (3.2 L) followed by water (2.8 L). The organic solution was azeotropically dried with 2-methyltetrahydrofuran by vacuum distillation to give a final volume of 2.0 L. The contents were heated to 80° C., stirred for 0.5 h then cooled to 20° C. over c. 3 h before heptane (2.0 L) was added over c. 3 h. The product was then collected by vacuum filtration and washed with a 1:1.4 mixture of 2-methyltetrahydrofuran and heptane (0.8 L) before drying under vacuum at 50° C. to deliver intermediate grade (11R)-14-chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene (245 g, 72.4%)

### Preparation of (11R)-14-Chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene, form 1

Intermediate grade (11R)-14-chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene (522.7 g) was stirred in a mixture of 30:70 v/v 1-propanol/heptane (1.57 L) at 20° C. for c.26 hours. The product was collected by vacuum filtration and washed with 30:70 v/v 1-propanol/heptane (1 L) before drying under vacuum at 50° C. to deliver (11R)-14-chloro-4,11-dimethyl-5-(oxan-4-yl)-8-oxa-2,5,6,12,16,17-hexaazatricyclo [11.3.1.0^{3,7}]heptadeca-1(16),3,6,13(17),14-pentaene, form 1 (485.8 g, 93%).

The compound of example 6 was subjected to form screening. In addition to form 1 (a non-solvated crystalline form), which may be produced as outlined above, a 1,4-dioxane solvate was isolated as a mixture with form 1 following slow evaporation of a solution of example 6 in 1,4-dioxane at room temperature over seven days, followed by rapid evaporation of remaining solutions under reduced pressure for 24 hours.

Form 1 exhibits a sharp endotherm at 223° C. and negligible weight loss up to 225° C. XRPD data for form 1 was acquired using a PANalytical X'Pert Pro powder diffractometer, model PW3040/60 using an X'Celerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a silicon wafer (zero background plate), resulting in a thin layer of powder. Peak positions were measured using Highscore software.

Characteristic XRPD angles form Form I produced by the route given above are recorded in Table 2. The margin of error is approximately ±0.2° 2θ for each of the peak assignments. Peak intensities may vary from sample to sample due to preferred orientation.

TABLE 2

| Peak | Position [° 2θ] |
|---|---|
| 1 | 8.7 |
| 2 | 10.1 |
| 3 | 10.5 |
| 4 | 13.9 |
| 5 | 15.9 |
| 6 | 17.4 |
| 7 | 18.2 |
| 8 | 18.7 |
| 9 | 19.6 |
| 10 | 25.3 |
| 11 | 27.0 |

### F. Biological Assays and Data

As stated above, the compounds of the present invention are LRRK2 kinase inhibitors, and may be useful in the treatment of diseases mediated by LRRK2. The biological activities and/or properties of the compounds of the present invention can be determined using any suitable assay, including assays for determining the activity of a candidate compound as a LRRK2 kinase inhibitor, as well as tissue and in vivo models.

### 1. Assays

a. Full Length G2019 Human LRRK2 Inhibition Mass Spectrometry Assay

This assay for Leucine Rich Repeat Kinase 2 (LRRK2) inhibition is based on the direct measurement of the peptide ‘LRRKtide’ (LRRKtide: RLGRDKYKT*LRQIRQ and “*” refers to the site of phosphorylation.) and phosphorylated ‘LRRKtide’ using a high throughput Rapid Fire mass spectrometry assay. Inhibitors are compounds that reduce the conversion of LRRKtide to phospho-LRRKtide.

### Human G2019 LRRK2 Plasmid Preparation

Primers used for PCR cloning:

pHTBV-F:
SEQ ID No: 1
LRRK2 wt-F1:
SEQ ID No: 2
LRRK2 wt-R1:
SEQ ID No: 3
LRRK2 wt-F2:
SEQ ID No: 4
LRRK2 wt-R2:
SEQ ID No: 5
LRRK2 wt-F3:
SEQ ID No: 6
pHTBV-R:
SEQ ID No: 7

pHTBV1-N-Flag-hu LRRK2 was generated by PCR amplifying the full length LRRK2 sequence with N terminal Flag tag from pcDNA3.1(+)_Human_LRRK2 (NCBI Reference Sequence: NP_940980.3) with the primers described above, and cloned into pHTBV1mcs3 vector between BamHI and KpnI sites.

The G2019 full length Flag-LRRK2 coding sequence is SEQ ID No: 8.

The translated amino acid sequence for human G2019 full length N terminal flag tagged LRRK2 protein is SEQ ID No: 9.

### Insect Cell Cultures

Sf9 insect cells (Invitrogen Life Technologies, Carlsbad, Calif.) were maintained at 27° C. in SF 900 II SFM in 500-ml shaker flasks (Erlenmeyer, Corning). The cells were maintained in exponential growth phase and subcultured twice per week. For larger volumes, cells were grown in 2-liter shake flasks (Erlenmeyer, Corning) 120 rpm at 27° C. in a shaking incubator, with agitation at 120 rpm.

### Generation of the BacMam Virus

To generate the recombinant BacMam virus, DH10Bac competent cells (10361-012, Invitrogen) were transformed with the genotypically normal human LRRK2 BacMam plasmid to generate the recombinant baculovirus DNA. The Sf9 insect cells were co-transfected with a mixture of recombinant baculovirus DNA and cellfectin (10362-100, Invitrogen). After 4 h of incubation at 27° C., the transfection medium was replaced with Sf-900 III SFM medium containing 5% HI FBS (Ser. No. 10/100,147, Invitrogen). The cells were further incubated for 4 days. The infected cell culture medium containing the baculovirus (P0 virus stock) was collected and amplified by further infecting the 200 ml Sf9 cells with 200-300 ul of the P0 virus stock.

### Quantification of BacMam Viral Titre by BacPAKRapid Titer

The viral titre, measured as plaque forming unit (pfu)/ml was determined using BacPAK Rapid Titer Kit (631406, Clontech) according to the manufacturer's protocol. The Sf9 cells seeded in 96-well plate with 3×10⁵ cells per well were incubated with serial dilution of the viral stocks for 1 h at 27° C., 50 μl methyl cellulose overlay was added to each well followed by 43-47 h incubation. The cells were then fixed in 4% paraformaldehyde (PFA). After blocking the cells with diluted normal goat serum, Mouse anti-gp64 antibody was added to the cells. After 30 min incubation, the cells were washed with phosphate buffered saline containing 0.2% Triton-X100 (PBST) and incubated for a further 30 min with goat anti-mouse antibody/HRP conjugate. This was followed by blue peroxidase substrate which detects the single infected cells and foci of infected cells by their dark blue color.

### Protein Expression & Purification

a) Expression of Flag Tagged Full Length G2019 Human LRRK2

HEK293 6E cells were incubated in a 37° C. incubator with a humidified atmosphere of 5% CO₂ on an orbital shaker rotating at 110 rpm in Freestyle 293 medium (Invitrogen 12338) supplemented with o.1% Pluronic F68 (Invitrogen 24040-032), 25 ug/ml Geneticin (Invitrogen 10131035) and 1× Glutamax (Gibco 35050). On the day of transduction, the cell viability was higher than 98% and the cell density was in the range of 1×10⁶˜1.5×10⁶ cells/ml. HEK293 6E cells were centrifuged at 1,000 rpm for 10 min, and then the cells were resuspended in fresh Freestyle 293 expression medium with 0.1% Pluronic F-68 (Invitrogen: 24040—but without the other additions, at a density of 1×10⁶ cells/ml. BacMam virus containing Flag-hu LRRK2 (genotypically normal) was centrifuged at 40,000 g for 2 hours, then resuspended in fresh Freestyle 293 expression medium. The resuspended virus was added into the cells at MOI of 10. The cells were incubated in a 37° C. incubator with a humidified atmosphere of 5% CO₂ in air on an orbital shaker rotating at 110 rpm. Cultures were harvested at approximately 48 hours post-transduction by centrifugation at 4,000 rpm for 20 min and pellets were frozen for purification.

b) Purification of Flag Tagged Full Length G2019 Human LRRK2

The cell pellet was resuspended in (20 mL/liter cell culture) lysis buffer (50 mM TrisHCl pH7.5 at 4° C., 500 mM NaCl, 0.5 mM EDTA, 0.1% TritonX-100, 10% glycerol, freshly add 2 mM DTT), with protease inhibitors (Roche: 04693132001) and benzonase (Merck Millipore: 70746-3CN) at recommended concentration suggested by suppliers. The suspended cells were lysed by sonication on ice for 30 min (2 secs on/4 sec off, 20% amplitude), and centrifuged at 10,000 rpm for 30 minutes at 4° C. The supernatant was incubated with 1 mL per litre of cell culture of anti-Flag magnetic beads (Sigma-Aldrich: M8823) at 4° C. for 3 hours, prior to washing the beads 3 times with 5 mL (5 column volumes) of binding buffer (50 mM Tris pH7.5@ 4° C., 500 mM NaCl, 0.5 mM EDTA, 0.1% TritonX-100, 10% glycerol, freshly add 2 mM DTT). The Flag tagged LRRK2 proteins were eluted with Elution buffer (50 mM Tris pH7.5@ 4° C., 500 mM NaCl, 0.5 mM EDTA, 0.1% TritonX-100, 10% glycerol, freshly add 2 mM DTT, 250 ug/ml Flag peptide (Sigma-Aldrich: F3290)) at 4° C. for 2 hours. Flag peptide was removed using Zeba Spin Desalting Columns, 7K MWCO (Thermo-Fisher: 89893) and the buffer containing the eluted LRRK2 proteins was exchanged into Storage Buffer (50 mM Tris pH7.5@4° C., 150 mM NaCl, 0.5 mM EDTA, 0.02% Triton X-100, 2 mM DTT and 50% Glycerol) using Amicon Ultra Centrifugal Filter Units (100 kD) (Merck: UFC910096). Fractions containing LRRK2 proteins were pooled, aliquoted and stored at −80° C. Protein concentration was determined by Bradford protein assay, and protein purity was analyzed by SDS-PAGE using NuPAGE Novex 4-12% Bis-Tris Protein Gels (Invitrogen: NP0322BOX).

### Assay Protocol

1) Compounds were dissolved in 100% DMSO at a concentration of 10 mM, and 11-point dose response curves generated using a 1 in 4 serial dilution in DMSO. 100 nL of this dilution series was then added to a 384 well, v bottom polypropylene plate (Greiner 781280), excluding columns 6 and 18. 100 nL of DMSO were added to column 6 to serve as the high control, while column 18 served as the low control containing assay buffer in place of the LRRK2 protein. Assay dilution gave a top final assay concentration of test compound of 100 μM
2) 5 μL of ‘enzyme solution’ containing 2× final assay concentration of purified recombinant Full length Flag-LRRK2 in assay buffer (50 mM Hepes, pH 7.2, 10 mM MgCl₂, 150 mM NaCl, 5% glycerol, 0.0025% Triton X-100 and 1 mM DTT) were added to all wells using a Multidrop Combi dispenser (ThermoFisher Scientifiec), giving a final assay concentration of 25 nM LRRK2 enzyme (final concentration may vary depending on specific activity of different enzyme batches).
3) 5 μL of substrate solution containing 50 μM LRRKtide peptide substrate and mM ATP was added to all wells of the plate using a Multidrop Combi dispenser giving a final assay concentration of 25 μM LRRKtide peptide and 2 mM ATP. Test plates were then incubated for 1 hour at room temperature. (Incubation may vary depending on rate and linearity of reaction with different enzyme batches).
4) 50 μl of 1% formic acid in laboratory grade water was added to all wells to quench the reaction, and plates were heat-sealed prior to centrifugation at 1348×g for 10 minutes. Test plates were then analysed on an Agilent RapidFire High Throughput solid phase extraction system coupled to AB Sciex API 4000 triple quadropole mass spectrometer with the following setting:

### RapidFire Settings:

Sip Height=2 mm, Aspirate=500 ms, Load time=3000 ms, Elution time=3000 ms, Requilibration=500 ms.
Flow rates: pump 1=1.5 mL/min, pump 2 1.25 mL/min pump 3=0.8 mL/min

Mass Spectrometer Settings:
LRRKtide Detection settings: Q1 mass 644.8 Da, Q3 mass 638.8, declustering potential 76 volts, collision energy 37 volts, CXP 34 volts.
Phospho-LRRKtide Detection settings: Q1 mass 671.4 Da, Q3 mass 638.8, Declustering potential 76 volts, Collision energy 37 volts, CXP 34 volts.
A C4 cartridge was used and running buffers were: A (aqueous) 0.1% formic acid in water B (organic) 0.1% formic acid, 80% acetonitrile, 20% water.
Collision gas: 12, Curtain gas: 25, Ion Source gas (1): 60, Ion Source gas (2): 60, Ion Spray Voltage: 5500, Temperature: 600, Interface Heater: ON.
Resolution Q1: low, Resolution Q3: low.
5) Data were analysed using ActivityBase software (IDBS). A percent conversion from LRRKtide to Phospho-LRRKtide was calculated using the following formula:

% conversion=(Phospho-LRRKtide product peak area/(Phospho-LRRKtide product peak area+LRRKtide substrate peak area))*100
A logistic 4 parameter curve fit was applied, using the following formula,

$$y = \frac{a - d}{1 + {( x \text{/} c )}^{b}} + d$$

Where x is the concentration of test sample, y is inhibition (%), a is the minimum, b is the Hill slope, c is the IC₅₀ and d is the maximum. From this, the pIC50 was derived (−log IC50).

b. Recombinant Cellular LRRK2 AlphaScreen Assay

To determine the activity of compounds against LRRK2 kinase in cells, the observed LRRK2 kinase-dependent modulation of LRRK2 Ser 935 phosphorylation (Dzamko et al., 2010, Biochem. J. 430: 405-413) was utilized to develop a quantitative 384 well plate-based immunoassay of LRRK2 Ser935 phosphorylation in the human neuroblastoma cell line SH-SY5Y, engineered to over-express recombinant full length LRRK2 protein.

### Assay Protocol

A BacMam virus expressing full length recombinant LRRK2 was purchased from Invitrogen and amplified by inoculation of SF-9 cells at MOI 0.3 for 4-5 days in Sf-900 III SFM medium supplemented with 3% fetal bovine serum. Infected cell cultures were then centrifuged at 2000 g for 20 minutes, viral supernatant titer determined by anti-gp64 plaque assay and stored at 4° C.

Affinity-purified anti-phospho LRRK2 Ser935 sheep polyclonal antibody (Dzamko et al., 2010, Biochem. J. 430: 405-413) was biotinylated by standard methods (PerkinElmer). Anti-LRRK2 rabbit polyclonal antibody was purchased from Novus Biologicals. AlphaScreen Protein A IgG Kit (including acceptor and donor beads) was purchased from Perkin Elmer.

SH-SY5Y cells were grown in DMEM/F12 medium with 10% dialysed fetal bovine serum and harvested by treatment with 0.5% trypsin-EDTA for 5 minutes at 37° C. followed by centrifugation at 1000 rpm for 4 minutes. The cell pellet was resuspended in Opti-MEM reduced serum media (Invitrogen) at 200,000 cells/ml and mixed with the BacMam LRRK2 virus at M01=50. 50 μl cell solutions were then dispensed to each well of a 384-well plate and incubated at 37° C., 5% CO₂ for 24 hours.

Compounds were prepared at 10× final assay concentration as 11 point 1 in 4 serial I dilutions in Opti-MEM reduced serum medium (Invitrogen 31985070) containing 1% DMSO. On the day of assay, 5.5 μl of the compound solutions were transferred from the compound plate to the cell assay plate to achieve a top final assay concentration of 10 uM. DMSO (0.1% final assay concentration) was added to column 6 and 2-(benzyloxy)-5-methyl-4-(1-methyl-1H-pyrazol-4-yl)-N-(pyridin-3-yl)benzamide (2 μM final assay concentration) was added to column 18 to serve as 0% and 100% inhibition controls, respectively.

Cells were incubated at 37° C., 5% CO₂ for 60 minutes. The medium was then removed and cells lysed by addition of 20 μl of cell lysis buffer (Cell Signaling Technology) and incubation at 4° C. for 20 minutes. 10 μl of antibody/acceptor bead mix [(1/1000 biotinylated-pS935 LRRK2 antibody, 1/1000 total-LRRK2 antibody, 1/100 Acceptor beads in AlphaScreen detection buffer (25 mM Hepes (pH 7.4), 0.5% Triton X-100, 1 mg/ml Dextran 500 and 0.1% BSA)] was then added to each well and plates incubated for 2 hours at ambient temperature in the dark. 10 μl of donor beads solution (1/33.3 donor beads in AlphaScreen detection buffer) was then added to each well. Following incubation for a further 2 hours at ambient temperature in the dark, plates were read on an EnVision™ plate reader at emission 520-620 nm with excitation 680 nm. Dose response curve data was based on a sigmoidal dose-response model.

c. Metabolic Stability Assay

Metabolic stability of compounds of the invention was evaluated in hepatocytes from human and preclinical species (e.g. rat). A test compound (0.5 μM) was incubated with 0.5 million viable hepatocytes at 37° C. in a 95% humidified incubator supplied with 5% CO₂. 7-ethoxycoumarin was used as positive control and was treated in the same manner as the test compound. The incubation samples were gently mixed at 150 rpm. Incubation medium was Williams' Medium E containing 2 mM GlutaMAX™ and 25 mM HEPES. Samples were removed from incubation at a specified time interval (tₓ): 0, 15, 30, 60, 90 and 120 min. Reactions were terminated by adding equal volume of ice-cold acetonitrile/methanol/acetic acid (80/20/1, v/v/v) containing an internal standard (e.g. 100 ng/mL to butamide as internal standard). The precipitated protein was pelleted by centrifugation and the supernatant was transferred to a new 96-well polypropylene plate for LC/MS/MS analysis. The instrument responses of the test compound and the internal standard were measured using a compound specific LC/MS/MS methodology. The percentage of remaining test compound is calculated from the peak area ratio between the remaining test compound after incubation (tₓ) and the test compound peak area at the beginning of incubation (tₓ=0 min).

The half-life (t_{1/2}) is calculated using the following equation:

t_{1/2}=−ln(2)/k

where k is the turn-over rate constant from regression of ln (percentage of remaining test compound) over tₓ.

Metabolic stability of a test compound is expressed as Intrinsic Clearance (Clᵢₙₜ) and is calculated from the half-life (t_{1/2}) using the following equation:

Clᵢₙₜ (ml/min/g)=(0.693/t_{1/2})×(ml per incubation/number of cells per incubation)×(number of cells/gram wet weight tissue)

Constants used to represent number of million cells/gram of wet tissue weight: 117.5 for human tissue and 108 for rat tissue.

### 2. Biological Data

Certain compounds of the invention were tested in the full length G2019 human LRRk2 inhibition mass spectrometry assay, the recombinant cellular LRRK2 alphaScreen assay and the metabolic stability assay.

The pIC50 value or the Clᵢₙₜ value from the assays for each tested compound either reported in at least one experiment or the average of multiple experiments. It is understood that the data described herein may have reasonable variations depending on the specific conditions and procedures used by the person conducting the experiments.

In the full length G2019 human LRRK2 inhibition mass spectrometry assay, the following compounds of the invention exhibited pIC₅₀≥5.0: Examples E1, E3, E5, E6, E7, E9, E11, E12, E13, E19, E20, E26, E28, E29, E30, E31, E32, E33, E34, E35, E36, E43, E45, E46, E47, E48, E49, E51, E52, E53, E54, E56, E57, E58, E60, E61, E62, E63, E64, E65, E66, E67, E68, E70, E84, E88, E89, E90, E104, E105, E106, E107, E109, E110, E112, E114, E116, E117, E120, E121, E123, E124, E127, E128, E130, E134, E136, E137, E138, E140, E141, E145, E146, E147, E154, E155, E180, E182, E183, E184, E185, E186, E187, E188, E189, E190, E191, E192, E193, E195, E197, E198, E199, E201, E203, E208, E209, E210, E211, E212, E213, E214, E215, E217, E218, E221, E222, E228, E236, E243, E244, E245, E246, E250, E251, E258, E259, E260, E261, E262, E263, E264, E265, E266, E270, E271, E272, E273, E274, E275, E278, E279, E280, E286, E287, E290, E291, E292, E293, E294, E295, E296, E298, E299, E301, E302, E303, E304, E305, E306, E307, E308, E309, E311, E315, E316, E317, E318, E319, E320, E323, E351, E352, E353, E354, E355, E356, E357, E358, E360, E363, E364, E365, E366, E367, E369, E370, E375, E376, E377, E378, E379, E382, E385, E386, E387, E388, E389, E390, E391, E394, E395, E396, E397, E399, E401, E403, E404, E405, E406, E409, E410, E411, E414, E415, E416, E417, E418, E419, E421, E422, E423, E424, E426, E427, E428, E429, E430, E432, E433, E444, E445, E446 and E447. The following compounds exhibited pIC₅₀≥7.0: Examples E1, E3, E5, E6, E11, E12, E13, E20, E28, E30, E31, E32, E35, E36, E45, E47, E48, E49, E51, E52, E54, E57, E58, E60, E61, E62, E64, E65, E66, E67, E68, E70, E84, E88, E90, E104, E105, E106, E107, E109, E110, E112, E114, E116, E117, E121, E123, E124, E127, E128, E130, E134, E136, E137, E140, E141, E145, E146, E154, E155, E180, E183, E184, E186, E187, E188, E191, E192, E195, E198, E201, E203, E208, E210, E213, E215, E218, E221, E222, E236, E243, E244, E245, E251, E258, E259, E260, E265, E266, E270, E272, E274, E278, E287, E291, E293, E295, E298, E301, E302, E304, E305, E306, E307, E308, E309, E311, E316, E317, E318, E320, E351, E352, E353, E354, E355, E357, E358, E364, E365, E366, E367, E369, E375, E376, E377, E378, E379, E382, E385, E386, E387, E388, E389, E390, E391, E394, E395, E396, E397, E399, E401, E403, E404, E405, E406, E410, E411, E414, E415, E416, E417, E418, E419, E421, E422, E423, E424, E427, E430, E432, E433, E444, E445, E446 and E447. The following compounds exhibited pIC50=4: Examples E181, E216 and E312.

In the recombinant cellular LRRK2 alphaScreen assay, the following compounds of the invention exhibited pIC₅₀≥5.0: Examples E1 through E188; E190 through E427; E430 through E440; E442 through E452. The following compounds exhibit pIC₅₀≥7.0: Examples E1, E2, E3, E5, E6, E8, E10, E11, E12, E13, E14, E15, E16, E17, E18, E20, E24, E25, E27, E28, E30, E32, E35, E38, E42, E44, E45, E46, E47, E48, E49, E50, E51, E52, E54, E57, E58, E59, E60, E61, E62, E64, E65, E66, E67, E68, E69, E70, E71, E72, E73, E80, E81, E82, E84, E86, E87, E88, E90, E91, E94, E95, E96, E98, E100, E101, E104, E105, E106, E107, E108, E109, E110, E111, E112, E116, E117, E118, E119, E121, E122, E123, E124, E125, E126, E127, E128, E130, E131, E132, E133, E134, E135, E136, E139, E140, E141, E142, E143, E144, E145, E146, E150, E151, E152, E153, E154, E158, E161, E162, E163, E165, E168, E169, E170, E172, E174, E175, E178, E179, E180, E181, E183, E184, E186, E187, E188, E191, E192, E194, E195, E198, E200, E201, E202, E203, E206, E207, E208, E210, E213, E215, E218, E219, E220, E221, E222, E223, E227, E228, E229, E230, E233, E234, E235, E236, E237, E238, E239, E241, E243, E244, E245, E247, E249, E251, E255, E257, E258, E259, E260, E265, E266, E269, E270, E274, E276, E278, E281, E282, E284, E287, E288, E289, E291, E293, E300, E301, E302, E303, <E304, E305, E306, E307, E308, E309, E310, E311, E312, E314, E316, E317, E318, E322, E325, E326, E328, E330, E331, E334, E335, E336, E337, E338, E339, E341, E342, E343, E345, E346, E347, E351, E353, E354, E357, E358, E359, E362, E363, E364, E365, E366, E367, E369, E371, E372, E373, E374, E375, E376, E377, E378, E379, E380, E382, E383, E384, E385, E386, E387, E389, E391, E392, E393, E394, E395, E396, E397, E399, E400, E401, E402, E403, E404, E406, E408, E410, E411, E412, E413, E415, E416, E417, E421, E422, E423, E427, E430, E431, E432, E433, E436, E437, E438, E440, E442, E443, E444, E445, E446, E447, E448, E450, E451 and E452. The following compounds exhibited pIC50<5.0: Examples E189, E428, E429, E441, racemic 14-chloro-4,11-dimethyl-5-{2-oxaspiro[3.3]heptan-6-yl}-8-oxa-2,5,6,12,16,17-hexaazatricyclo[11.3.1.0^{3,7}]heptadeca-1(17),3,6,13,15-pentaene and 3-methyl-8-(prop-1-en-2-yl)-2-(tetrahydro-2H-pyran-4-yl)-2,4,10,11,12,13-hexahydro-5,9-(azeno)pyrazolo[3,4-b][1,4,6,10]oxatriazacyclotridecine.

In the metabolic stability assay (in human hepatocytes), the following compounds of the invention exhibited Clᵢₙₜ (human)≤10.0: Examples E1, E3, E6, E10, E11, E12, E14, E15, E16, E17, E18, E20, E24, E27, E28, E29, E30, E32, E34, E35, E44, E45, E46, E47, E48, E52, E53, E54, E57, E59, E60, E61, E62, E63, E64, E65, E66, E67, E68, E83, E84, E88, E95, E98, E101, E102, E104, E106, E107, E123, E124, E134, E136, E145, E146, E178, E181, E183, E184, E187, E191, E195, E196, E202, E203, E210, E213, E220, E222, E229, E230, E233, E234, E235, E236, E238, E243, E244, E249, E251, E261, E265, E266, E270, E276, E281, E284, E285, E288, E289, E290, E291, E292, E293, E297, E298, E301, E302, E303, E305, E306, E311, E315, E330, E338, E339, E343, E345, E359, E366, E367, E369, E371, E380, E393, E395, E400, E406 and E417. The following compounds exhibited Clᵢₙₜ≤3.0 (human): Examples E1, E3, E6, E10, E11, E12, E14, E15, E16, E17, E18, E20, E24, E27, E28, E29, E30, E32, E34, E35, E44, E45, E46, E47, E48, E52, E53, E54, E57, E59, E60, E61, E62, E63, E64, E65, E66, E67, E68, E83, E84, E88, E95, E98, E101, E102, E104, E106, E107, E123, E124, E134, E136, E145, E146, E178, E181, E183, E184, E187, E191, E195, E196, E202, E203, E210, E213, E229, E230, E233, E234, E235, E236, E238, E243, E244, E249, E251, E265, E266, E270, E276, E281, E284, E285, E288, E289, E290, E291, E292, E293, E297, E298, E301, E302, E303, E305, E306, E311, E315, E330, E338, E339, E343, E345, E359, E366, E367, E369, E371, E380, E393, E395, E406 and E417. The compound of example E362 exhibited Clᵢₙₜ>10 (human).

Sequence Listing
SEQ ID NO: 1 Primers used for PCR cloning of Human G2019LRRK2
plasmids preparation: pHTBV-F
5′-GATCTCGACGGGCGCGGATCCACCATGGATTACAAGGATGACGACGAT-3′
SEQ ID NO: 2 Primers used for PCR cloning of Human G2019LRRK2
plasmids preparation: LRRK2 wt-F1
5′-CATGGATTACAAGGATGACGACGATAAGATGGCTAGTGGCAGCTGTCAG-3′
SEQ ID NO: 3 Primers used for PCR cloning of Human G2019LRRK2
plasmids preparation: LRRK2 wt-R1
5′-GTTCACGAGATCCACTATTCAGTAAGAGTTCCACCAATTTGGGACTG-3′
SEQ ID NO: 4 Primers used for PCR cloning of Human G2019LRRK2
plasmids preparation: LRRK2 wt-F2
5′-GAATAGTGGATCTCGTGAACAAG-3′
SEQ ID NO: 5 Primers used for PCR cloning of Human G2019LRRK2
plasmids preparation: LRRK2 wt-R2
5′-GTCAGACAAACTGCTTGGAACCAGC-3′
SEQ ID NO: 6 Primers used for PCR cloning of Human G2019LRRK2
plasmids preparation: LRRK2 wt-F3
5′-CTGGTTCCAAGCAGTTTGTCTGACCACAGGCCTGTGATAG-3′
SEQ ID NO: 7 Primers used for PCR cloning of Human G2019LRRK2
plasmids preparation: pHTBV-R
5′-GTTCTAGCCAAGCTTGGTACCCTATTACTCAACAGATGTTCGTCTC-3′
SEQ ID NO: 8 G2019 full length Flag-LRRK2 coding sequence
atggattacaaggatgacgacgataagATGGCTAGTGGCAGCTGTCAGGGGTGCGAAGAGGACGAGGAAAC
TCTGAAGAAGTTGATAGTCAGGCTGAACAATGTCCAGGAAGGAAAACAGATAGAAACGCTGGTCCAAATCC
TGGAGGATCTGCTGGTGTTCACGTACTCCGAGCACGCCTCCAAGTTATTTCAAGGCAAAAATATCCATGTG
CCTCTGTTGATCGTCTTGGACTCCTATATGAGAGTCGCGAGTGTGCAGCAGGTGGGTTGGTCACTTCTGTG
CAAATTAATAGAAGTCTGTCCAGGTACAATGCAAAGCTTAATGGGACCCCAGGATGTTGGAAATGATTGGG
AAGTCCTTGGTGTTCACCAATTGATTCTTAAAATGCTAACAGTTCATAATGCCAGTGTAAACTTGTCAGTG
ATTGGACTGAAGACCTTAGATCTCCTCCTAACTTCAGGTAAAATCACCTTGCTGATACTGGATGAAGAAAG
TGATATTTTCATGTTAATTTTTGATGCCATGCACTCATTTCCAGCCAATGATGAAGTCCAGAAACTTGGAT
GCAAAGCTTTACATGTGCTGTTTGAGAGAGTCTCAGAGGAGCAACTGACTGAATTTGTTGAGAACAAAGAT
TATATGATATTGTTAAGTGCGTTAACAAATTTTAAAGATGAAGAGGAAATTGTGCTTCATGTGCTGCATTG
TTTACATTCCCTAGCGATTCCTTGCAATAATGTGGAAGTCCTCATGAGTGGCAATGTCAGGTGTTATAATA
TTGTGGTGGAAGCTATGAAAGCATTCCCTATGAGTGAAAGAATTCAAGAAGTGAGTTGCTGTTTGCTCCAT
AGGCTTACATTAGGTAATTTTTTCAATATCCTGGTATTAAACGAAGTCCATGAGTTTGTGGTGAAAGCTGT
GCAGCAGTACCCAGAGAATGCAGCATTGCAGATCTCAGCGCTCAGCTGTTTGGCCCTCCTCACTGAGACTA
TTTTCTTAAATCAAGATTTAGAGGAAAAGAATGAGAATCAAGAGAATGATGATGAGGGGGAAGAAGATAAA
TTGTTTTGGCTGGAAGCCTGTTACAAAGCATTAACGTGGCATAGAAAGAACAAGCACGTGCAGGAGGCCGC
ATGCTGGGCACTAAATAATCTCCTTATGTACCAAAACAGTTTACATGAGAAGATTGGAGATGAAGATGGCC
ATTTCCCAGCTCATAGGGAAGTGATGCTCTCCATGCTGATGCATTCTTCATCAAAGGAAGTTTTCCAGGCA
TCTGCGAATGCATTGTCAACTCTCTTAGAACAAAATGTTAATTTCAGAAAAATACTGTTATCAAAAGGAAT
ACACCTGAATGTTTTGGAGTTAATGCAGAAGCATATACATTCTCCTGAAGTGGCTGAAAGTGGCTGTAAAA
TGCTAAATCATCTTTTTGAAGGAAGCAACACTTCCCTGGATATAATGGCAGCAGTGGTCCCCAAAATACTA
ACAGTTATGAAACGTCATGAGACATCATTACCAGTGCAGCTGGAGGCGCTTCGAGCTATTTTACATTTTAT
AGTGCCTGGCATGCCAGAAGAATCCAGGGAGGATACAGAATTTCATCATAAGCTAAATATGGTTAAAAAAC
AGTGTTTCAAGAATGATATTCACAAACTGGTCCTAGCAGCTTTGAACAGGTTCATTGGAAATCCTGGGATT
CAGAAATGTGGATTAAAAGTAATTTCTTCTATTGTACATTTTCCTGATGCATTAGAGATGTTATCCCTGGA
AGGTGCTATGGATTCAGTGCTTCACACACTGCAGATGTATCCAGATGACCAAGAAATTCAGTGTCTGGGTT
TAAGTCTTATAGGATACTTGATTACAAAGAAGAATGTGTTCATAGGAACTGGACATCTGCTGGCAAAAATT
CTGGTTTCCAGCTTATACCGATTTAAGGATGTTGCTGAAATACAGACTAAAGGATTTCAGACAATCTTAGC
AATCCTCAAATTGTCAGCATCTTTTTCTAAGCTGCTGGTGCATCATTCATTTGACTTAGTAATATTCCATC
AAATGTCTTCCAATATCATGGAACAAAAGGATCAACAGTTTCTAAACCTCTGTTGCAAGTGTTTTGCAAAA
GTAGCTATGGATGATTACTTAAAAAATGTGATGCTAGAGAGAGCGTGTGATCAGAATAACAGCATCATGGT
TGAATGCTTGCTTCTATTGGGAGCAGATGCCAATCAAGCAAAGGAGGGATCTTCTTTAATTTGTCAGGTAT
GTGAGAAAGAGAGCAGTCCCAAATTGGTGGAACTCTTACTGAATAGTGGATCTCGTGAACAAGATGTACGA
AAAGCGTTGACGATAAGCATTGGGAAAGGTGACAGCCAGATCATCAGCTTGCTCTTAAGGAGGCTGGCCCT
GGATGTGGCCAACAATAGCATTTGCCTTGGAGGATTTTGTATAGGAAAAGTTGAACCTTCTTGGCTTGGTC
CTTTATTTCCAGATAAGACTTCTAATTTAAGGAAACAAACAAATATAGCATCTACACTAGCAAGAATGGTG
ATCAGATATCAGATGAAAAGTGCTGTGGAAGAAGGAACAGCCTCAGGCAGCGATGGAAATTTTTCTGAAGA
TGTGCTGTCTAAATTTGATGAATGGACCTTTATTCCTGACTCTTCTATGGACAGTGTGTTTGCTCAAAGTG
ATGACCTGGATAGTGAAGGAAGTGAAGGCTCATTTCTTGTGAAAAAGAAATCTAATTCAATTAGTGTAGGA
GAATTTTACCGAGATGCCGTATTACAGCGTTGCTCACCAAATTTGCAAAGACATTCCAATTCCTTGGGGCC
CATTTTTGATCATGAAGATTTACTGAAGCGAAAAAGAAAAATATTATCTTCAGATGATTCACTCAGGTCAT
CAAAACTTCAATCCCATATGAGGCATTCAGACAGCATTTCTTCTCTGGCTTCTGAGAGAGAATATATTACA
TCACTAGACCTTTCAGCAAATGAACTAAGAGATATTGATGCCCTAAGCCAGAAATGCTGTATAAGTGTTCA
TTTGGAGCATCTTGAAAAGCTGGAGCTTCACCAGAATGCACTCACGAGCTTTCCACAACAGCTATGTGAAA
CTCTGAAGAGTTTGACACATTTGGACTTGCACAGTAATAAATTTACATCATTTCCTTCTTATTTGTTGAAA
ATGAGTTGTATTGCTAATCTTGATGTCTCTCGAAATGACATTGGACCCTCAGTGGTTTTAGATCCTACAGT
GAAATGTCCAACTCTGAAACAGTTTAACCTGTCATATAACCAGCTGTCTTTTGTACCTGAGAACCTCACTG
ATGTGGTAGAGAAACTGGAGCAGCTCATTTTAGAAGGAAATAAAATATCAGGGATATGCTCCCCCTTGAGA
CTGAAGGAACTGAAGATTTTAAACCTTAGTAAGAACCACATTTCATCCCTATCAGAGAACTTTCTTGAGGC
TTGTCCTAAAGTGGAGAGTTTCAGTGCCAGAATGAATTTTCTTGCTGCTATGCCTTTCTTGCCTCCTTCTA
TGACAATCCTAAAATTATCTCAGAACAAATTTTCCTGTATTCCAGAAGCAATTTTAAATCTTCCACACTTG
CGGTCTTTAGATATGAGCAGCAATGATATTCAGTACCTACCAGGTCCCGCACACTGGAAATCTTTGAACTT
AAGGGAACTCTTATTTAGCCATAATCAGATCAGCATCTTGGACTTGAGTGAAAAAGCATATTTATGGTCTA
GAGTAGAGAAACTGCATCTTTCTCACAATAAACTGAAAGAGATTCCTCCTGAGATTGGCTGTCTTGAAAAT
CTGACATCTCTGGATGTCAGTTACAACTTGGAACTAAGATCCTTTCCCAATGAAATGGGGAAATTAAGCAA
AATATGGGATCTTCCTTTGGATGAACTGCATCTTAACTTTGATTTTAAACATATAGGATGTAAAGCCAAAG
ACATCATAAGGTTTCTTCAACAGCGATTAAAAAAGGCTGTGCCTTATAACCGAATGAAACTTATGATTGTG
GGAAATACTGGGAGTGGTAAAACCACCTTATTGCAGCAATTAATGAAAACCAAGAAATCAGATCTTGGAAT
GCAAAGTGCCACAGTTGGCATAGATGTGAAAGACTGGCCTATCCAAATAAGAGACAAAAGAAAGAGAGATC
TCGTCCTAAATGTGTGGGATTTTGCAGGTCGTGAGGAATTCTATAGTACTCATCCCCATTTTATGACGCAG
CGAGCATTGTACCTTGCTGTCTATGACCTCAGCAAGGGACAGGCTGAAGTTGATGCCATGAAGCCTTGGCT
CTTCAATATAAAGGCTCGCGCTTCTTCTTCCCCTGTGATTCTCGTTGGCACACATTTGGATGTTTCTGATG
AGAAGCAACGCAAAGCCTGCATGAGTAAAATCACCAAGGAACTCCTGAATAAGCGAGGGTTCCCTGCCATA
CGAGATTACCACTTTGTGAATGCCACCGAGGAATCTGATGCTTTGGCAAAACTTCGGAAAACCATCATAAA
CGAGAGCCTTAATTTCAAGATCCGAGATCAGCTTGTTGTTGGACAGCTGATTCCAGACTGCTATGTAGAAC
TTGAAAAAATCATTTTATCGGAGCGTAAAAATGTGCCAATTGAATTTCCCGTAATTGACCGGAAACGATTA
TTACAACTAGTGAGAGAAAATCAGCTGCAGTTAGATGAAAATGAGCTTCCTCACGCAGTTCACTTTCTAAA
TGAATCAGGAGTCCTTCTTCATTTTCAAGACCCAGCACTGCAGTTAAGTGACTTGTACTTTGTGGAACCCA
AGTGGCTTTGTAAAATCATGGCACAGATTTTGACAGTGAAAGTGGAAGGTTGTCCAAAACACCCTAAGGGA
ATTATTTCGCGTAGAGATGTGGAAAAATTTCTTTCAAAGAAAAGGAAATTTCCAAAGAACTACATGTCACA
GTATTTTAAGCTCCTAGAAAAATTCCAGATTGCTTTGCCAATAGGAGAAGAATATTTGCTGGTTCCAAGCA
GTTTGTCTGACCACAGGCCTGTGATAGAGCTTCCCCATTGTGAGAACTCTGAAATTATCATCCGACTATAT
GAAATGCCTTATTTTCCAATGGGATTTTGGTCAAGATTAATCAATCGATTACTTGAGATTTCACCTTACAT
GCTTTCAGGGAGAGAACGAGCACTTCGCCCAAACAGAATGTATTGGCGACAAGGCATTTACTTAAATTGGT
CTCCTGAAGCTTATTGTCTGGTAGGATCTGAAGTCTTAGACAATCATCCAGAGAGTTTCTTAAAAATTACA
GTTCCTTCTTGTAGAAAAGGCTGTATTCTTTTGGGCCAAGTTGTGGACCACATTGATTCTCTCATGGAAGA
ATGGTTTCCTGGGTTGCTGGAGATTGATATTTGTGGTGAAGGAGAAACTCTGTTGAAGAAATGGGCATTAT
ATAGTTTTAATGATGGTGAAGAACATCAAAAAATCTTACTTGATGACTTGATGAAGAAAGCAGAGGAAGGA
GATCTCTTAGTAAATCCAGATCAACCAAGGCTCACCATTCCAATATCTCAGATTGCCCCTGACTTGATTTT
GGCTGACCTGCCTAGAAATATTATGTTGAATAATGATGAGTTGGAATTTGAACAAGCTCCAGAGTTTCTCC
TAGGTGATGGCAGTTTTGGATCAGTTTACCGAGCAGCCTATGAAGGAGAAGAAGTGGCTGTGAAGATTTTT
AATAAACATACATCACTCAGGCTGTTAAGACAAGAGCTTGTGGTGCTTTGCCACCTCCACCACCCCAGTTT
GATATCTTTGCTGGCAGCTGGGATTCGTCCCCGGATGTTGGTGATGGAGTTAGCCTCCAAGGGTTCCTTGG
ATCGCCTGCTTCAGCAGGACAAAGCCAGCCTCACTAGAACCCTACAGCACAGGATTGCACTCCACGTAGCT
GATGGTTTGAGATACCTCCACTCAGCCATGATTATATACCGAGACCTGAAACCCCACAATGTGCTGCTTTT
CACACTGTATCCCAATGCTGCCATCATTGCAAAGATTGCTGACTACGGCATTGCTCAGTACTGCTGTAGAA
TGGGGATAAAAACATCAGAGGGCACACCAGGGTTTCGTGCACCTGAAGTTGCCAGAGGAAATGTCATTTAT
AACCAACAGGCTGATGTTTATTCATTTGGTTTACTACTCTATGACATTTTGACAACTGGAGGTAGAATAGT
AGAGGGTTTGAAGTTTCCAAATGAGTTTGATGAATTAGAAATACAAGGAAAATTACCTGATCCAGTTAAAG
AATATGGTTGTGCCCCATGGCCTATGGTTGAGAAATTAATTAAACAGTGTTTGAAAGAAAATCCTCAAGAA
AGGCCTACTTCTGCCCAGGTCTTTGACATTTTGAATTCAGCTGAATTAGTCTGTCTGACGAGACGCATTTT
ATTACCTAAAAACGTAATTGTTGAATGCATGGTTGCTACACATCACAACAGCAGGAATGCAAGCATTTGGC
TGGGCTGTGGGCACACCGACAGAGGACAGCTCTCATTTCTTGACTTAAATACTGAAGGATACACTTCTGAG
GAAGTTGCTGATAGTAGAATATTGTGCTTAGCCTTGGTGCATCTTCCTGTTGAAAAGGAAAGCTGGATTGT
GTCTGGGACACAGTCTGGTACTCTCCTGGTCATCAATACCGAAGATGGGAAAAAGAGACATACCCTAGAAA
AGATGACTGATTCTGTCACTTGTTTGTATTGCAATTCCTTTTCCAAGCAAAGCAAACAAAAAAATTTTCTT
TTGGTTGGAACCGCTGATGGCAAGTTAGCAATTTTTGAAGATAAGACTGTTAAGCTTAAAGGAGCTGCTCC
TTTGAAGATACTAAATATAGGAAATGTCAGTACTCCATTGATGTGTTTGAGTGAATCCACAAATTCAACGG
AAAGAAATGTAATGTGGGGAGGATGTGGCACAAAGATTTTCTCCTTTTCTAATGATTTCACCATTCAGAAA
CTCATTGAGACAAGAACAAGCCAACTGTTTTCTTATGCAGCTTTCAGTGATTCCAACATCATAACAGTGGT
GGTAGACACTGCTCTCTATATTGCTAAGCAAAATAGCCCTGTTGTGGAAGTGTGGGATAAGAAAACTGAAA
AACTCTGTGGACTAATAGACTGCGTGCACTTTTTAAGGGAGGTAATGGTAAAAGAAAACAAGGAATCAAAA
CACAAAATGTCTTATTCTGGGAGAGTGAAAACCCTCTGCCTTCAGAAGAACACTGCTCTTTGGATAGGAAC
TGGAGGAGGCCATATTTTACTCCTGGATCTTTCAACTCGTCGACTTATACGTGTAATTTACAACTTTTGTA
ATTCGGTCAGAGTCATGATGACAGCACAGCTAGGAAGCCTTAAAAATGTCATGCTGGTATTGGGCTACAAC
CGGAAAAATACTGAAGGTACACAAAAGCAGAAAGAGATACAATCTTGCTTGACCGTTTGGGACATCAATCT
TCCACATGAAGTGCAAAATTTAGAAAAACACATTGAAGTGAGAAAAGAATTAGCTGAAAAAATGAGACGAA
CATCTGTTGAGTAA
SEQ ID NO: 9 Translated protein sequence for human G2019 full length
LRRK2 flag tagged protein
MDYKDDDDKMASGSCQGCEEDEETLKKLIVRLNNVQEGKQIETLVQILEDLLVFTYSEHASKLFQGKNIHV
PLLIVLDSYMRVASVQQVGWSLLCKLIEVCPGTMQSLMGPQDVGNDWEVLGVHQLILKMLTVHNASVNLSV
IGLKTLDLLLTSGKITLLILDEESDIFMLIFDAMHSFPANDEVQKLGCKALHVLFERVSEEQLTEFVENKD
YMILLSALTNFKDEEEIVLHVLHCLHSLAIPCNNVEVLMSGNVRCYNIVVEAMKAFPMSERIQEVSCCLLH
RLTLGNFFNILVLNEVHEFVVKAVQQYPENAALQISALSCLALLTETIFLNQDLEEKNENQENDDEGEEDK
LFWLEACYKALTWHRKNKHVQEAACWALNNLLMYQNSLHEKIGDEDGHFPAHREVMLSMLMHSSSKEVFQA
SANALSTLLEQNVNFRKILLSKGIHLNVLELMQKHIHSPEVAESGCKMLNHLFEGSNTSLDIMAAVVPKIL
TVMKRHETSLPVQLEALRAILHFIVPGMPEESREDTEFHHKLNMVKKQCFKNDIHKLVLAALNRFIGNPGI
QKCGLKVISSIVHFPDALEMLSLEGAMDSVLHTLQMYPDDQEIQCLGLSLIGYLITKKNVFIGTGHLLAKI
LVSSLYRFKDVAEIQTKGFQTILAILKLSASFSKLLVHHSFDLVIFHQMSSNIMEQKDQQFLNLCCKCFAK
VAMDDYLKNVMLERACDQNNSIMVECLLLLGADANQAKEGSSLICQVCEKESSPKLVELLLNSGSREQDVR
KALTISIGKGDSQIISLLLRRLALDVANNSICLGGFCIGKVEPSWLGPLFPDKTSNLRKQTNIASTLARMV
IRYQMKSAVEEGTASGSDGNFSEDVLSKFDEWTFIPDSSMDSVFAQSDDLDSEGSEGSFLVKKKSNSISVG
EFYRDAVLQRCSPNLQRHSNSLGPIFDHEDLLKRKRKILSSDDSLRSSKLQSHMRHSDSISSLASEREYIT
SLDLSANELRDIDALSQKCCISVHLEHLEKLELHQNALTSFPQQLCETLKSLTHLDLHSNKFTSFPSYLLK
MSCIANLDVSRNDIGPSVVLDPTVKCPTLKQFNLSYNQLSFVPENLTDVVEKLEQLILEGNKISGICSPLR
LKELKILNLSKNHISSLSENFLEACPKVESFSARMNFLAAMPFLPPSMTILKLSQNKFSCIPEAILNLPHL
RSLDMSSNDIQYLPGPAHWKSLNLRELLFSHNQISILDLSEKAYLWSRVEKLHLSHNKLKEIPPEIGCLEN
LTSLDVSYNLELRSFPNEMGKLSKIWDLPLDELHLNFDFKHIGCKAKDIIRFLQQRLKKAVPYNRMKLMIV
GNTGSGKTTLLQQLMKTKKSDLGMQSATVGIDVKDWPIQIRDKRKRDLVLNVWDFAGREEFYSTHPHFMTQ
RALYLAVYDLSKGQAEVDAMKPWLFNIKARASSSPVILVGTHLDVSDEKQRKACMSKITKELLNKRGFPAI
RDYHFVNATEESDALAKLRKTIINESLNFKIRDQLVVGQLIPDCYVELEKIILSERKNVPIEFPVIDRKRL
LQLVRENQLQLDENELPHAVHFLNESGVLLHFQDPALQLSDLYFVEPKWLCKIMAQILTVKVEGCPKHPKG
IISRRDVEKFLSKKRKFPKNYMSQYFKLLEKFQIALPIGEEYLLVPSSLSDHRPVIELPHCENSEIIIRLY
EMPYFPMGFWSRLINRLLEISPYMLSGRERALRPNRMYWRQGIYLNWSPEAYCLVGSEVLDNHPESFLKIT
VPSCRKGCILLGQVVDHIDSLMEEWFPGLLEIDICGEGETLLKKWALYSFNDGEEHQKILLDDLMKKAEEG
DLLVNPDQPRLTIPISQIAPDLILADLPRNIMLNNDELEFEQAPEFLLGDGSFGSVYRAAYEGEEVAVKIF
NKHTSLRLLRQELVVLCHLHHPSLISLLAAGIRPRMLVMELASKGSLDRLLQQDKASLTRTLQHRIALHVA
DGLRYLHSAMIIYRDLKPHNVLLFTLYPNAAIIAKIADYGIAQYCCRMGIKTSEGTPGFRAPEVARGNVIY
NQQADVYSFGLLLYDILTTGGRIVEGLKFPNEFDELEIQGKLPDPVKEYGCAPWPMVEKLIKQCLKENPQE
RPTSAQVFDILNSAELVCLTRRILLPKNVIVECMVATHHNSRNASIWLGCGHTDRGQLSFLDLNTEGYTSE
EVADSRILCLALVHLPVEKESWIVSGTQSGTLLVINTEDGKKRHTLEKMTDSVTCLYCNSFSKQSKQKNFL
LVGTADGKLAIFEDKTVKLKGAAPLKILNIGNVSTPLMCLSESTNSTERNVMWGGCGTKIFSFSNDFTIQK
LIETRTSQLFSYAAFSDSNIITVVVDTALYIAKQNSPVVEVWDKKTEKLCGLIDCVHFLREVMVKENKESK
HKMSYSGRVKTLCLQKNTALWIGTGGGHILLLDLSTRRLIRVIYNFCNSVRVMMTAQLGSLKNVMLVLGYN
RKNTEGTQKQKEIQSCLTVWDINLPHEVQNLEKHIEVRKELAEKMRRTSVE
SEQ ID NO: 10: ′LRRKtide′ peptide
H-RLGRDKYKTLRQIRQ-OH

1-33. (canceled)

34. A compound of Formula (I) 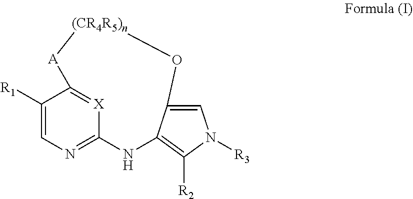
or a pharmaceutically acceptable salt thereof, wherein
X is CH or N;
n is 2, 3, 4 or 5;
A is O or NR^{a}, wherein
R^{a} is
H;
C₁₋₄alkyl optionally substituted with one to three substituents independently selected from the group consisting of halo, hydroxyl and C₁₋₃alkoxyl, wherein C₁₋₃alkoxyl is optionally substituted with one to three halo substituents;
C₃₋₆cycloalkyl optionally substituted with one to three substituents independently selected from the group consisting of halo, hydroxyl and C₁₋₃alkoxyl; or
four to seven-membered heterocyclyl ring having one or two heteroatom ring members independently selected from O and N, and the heterocyclyl ring is optionally substituted with one to three substituents independently selected from halo and C₁₋₃alkyl, which C₁₋₃alky is optionally substituted with one to three halo substituents;
R₁ is
1) H, halo, CN;
2) C₁₋₄alkyl optionally substituted with one to three substituents independently selected from the group consisting of halo, hydroxyl and C₁₋₃alkoxyl, which C₁₋₃alkoxyl is optionally substituted with one to three halo substituents;
3) C₂₋₆alkenyl optionally substituted with one to three halo or C₁₋₃alkyl, which C₁₋₃alkyl is optionally substituted with one to three halo substituents;
4) C₂₋₆alkynyl optionally substituted with one to three C₁₋₃alkyl substituents, which C₁₋₃alkyl is optionally substituted with one to three halo substituents;
5) C₁₋₄alkoxyl optionally substituted with one to three halo substituents;
6) C₃₋₆cycloalkyl optionally substituted with one to three substituents independently selected from the group consisting of halo, hydroxyl, C₁₋₃alkoxyl and C₁₋₃alkyl, wherein C₁₋₃alkoxyl and C₁₋₃alkyl are optionally substituted with one to three halo substituents;
7) —OC₃₋₆cycloalkyl optionally substituted with one to three substituents independently selected from the group consisting of halo, hydroxyl, C₁₋₃alkoxyl and C₁₋₃alkyl;
8) four to seven-membered heterocyclyl ring having one or two heteroatom ring members independently selected from O and N, and the heterocyclyl ring is optionally substituted with one to three substituents independently selected from the group consisting of halo, hydroxyl, C₁₋₃alkoxyl and C₁₋₃alkyl;
9) —O-heterocyclyl ring having one or two heteroatom ring members independently selected from O and N, wherein the heterocyclyl ring is a four to seven-membered ring optionally substituted with one to three substituents independently selected from the group consisting of halo, hydroxyl, C₁₋₃alkoxyl and C₁₋₃alkyl; or
10) —SC₁₋₄alkyl optionally substituted with one to three halo substituents;
R₂ is
H, halo, CN;
C₁₋₄alkoxyl optionally substituted with one to three halo substituents;
C₁₋₄alkyl optionally substituted with one to three substituents independently selected from the group consisting of halo, hydroxyl and C₁₋₃alkoxyl, which C₁₋₃alkoxyl is optionally substituted with one to three halo substituents; or
C₃₋₆cycloalkyl optionally substituted with one to three substituents independently selected from the group consisting of halo, hydroxyl and C₁₋₃alkoxyl;
R₃ is
1) H;
2) —CO—Z, wherein Z is selected from the group consisting of
four to seven-membered heterocyclyl ring having one or two heteroatom ring members independently selected from O and N;
C₃₋₆cycloalkyl optionally substituted with one to three substituents independently selected from the group consisting of halo, hydroxyl, C₁₋₃alkyl, and C₁₋₃alkoxyl; and
C₁₋₆alkyl optionally substituted with one to three substituents independently selected from halo and C₁₋₃alkoxyl;
3) four to seven-membered heterocyclyl ring having one or two heteroatom ring members independently selected from O and N, and the heterocyclyl ring is optionally substituted with one to three substituents independently selected from the group consisting of
halo;
cyano;
C₁₋₃alkyl optionally substituted with one to three substituents independently selected from halo, hydroxyl and C₁₋₃alkoxyl;
C₁₋₃alkoxyl optionally substituted with one to three substituents independently selected from halo, hydroxyl and C₁₋₃alkoxyl; and
four to seven-membered heterocyclyl ring having one or two heteroatom ring members independently selected from O and N, and the heterocyclyl ring is optionally substituted with one to three substituents independently selected from the group consisting of halo, C₁₋₃alkyl and C₁₋₃alkoxyl;
4) C₁₋₆alkyl optionally substituted with one to three substituents independently selected from the group consisting of
CN, hydroxyl, halo;
C₁₋₄alkoxyl optionally substituted with one to three substituents independently selected from halo, hydroxyl and C₁₋₃ alkoxyl;
—CO-Q, wherein Q is C₁₋₄alkoxyl, hydroxyl or NR_{c}R_{d}, wherein R_{c} and R_{d} are each independently H or C₁₋₄alkyl; 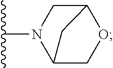 [SMILES: *N1CC2CC1CO2]
and
four to seven-membered heterocyclyl ring having one or two heteroatom ring members independently selected from O and N, wherein the heterocyclyl ring is optionally substituted with one to three halo substituents;
5) C₃₋₇cycloalkyl optionally substituted with one to three substituents independently selected from the group consisting of
halo, hydroxyl; 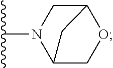 [SMILES: *N1CC2CC1CO2]
C₁₋₃alkyl optionally substituted with one to three substituents independently selected from the group consisting of halo, hydroxyl and C₁₋₃alkoxyl;
C₁₋₄alkoxyl optionally substituted with one to three substituents independently selected from the group consisting of halo, hydroxyl and C₁₋₃alkoxyl; and
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃ alkyl, and C₁₋₃ alkoxyl;
6) C-linked 7-9 membered bridged cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
7) C-linked 7-10 membered spirane cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl; or
8) C-linked 6-9 membered fused cyclyl ring optionally having one or two heteroatom ring members independently selected from O and N, optionally substituted with one to three substituents independently selected from the group consisting of halo, hydroxyl, C₁₋₃alkyl and C₁₋₃alkoxyl;
R₄ and R₅, at each occurrence, are each independently selected from the group consisting of
H, halo, hydroxyl;
C₁₋₄alkyl optionally substituted with one to three substituents independently selected from the group consisting of halo, C₁₋₄alkoxy, OC₁₋₄haloalkyl, and four to seven-membered heterocyclyl ring having one or two heteroatom ring members independently selected from O and N;
C₃₋₆cycloalkyl optionally substituted with one to three substituents independently selected from the group consisting of halo, hydroxyl and C₁₋₄ alkoxyl;
four to seven-membered heterocyclyl ring having one or two heteroatom ring members independently selected from O and N, wherein the heterocyclyl ring is optionally substituted with one to three substituents independently selected from the group consisting of halo, hydroxyl, C₁₋₃alkyl, and C₁₋₃alkoxyl; and
C₁₋₄alkoxyl optionally substituted with one to three substituents independently selected from the group consisting of halo, hydroxyl and C₁₋₄alkoxyl.

35. A compound or a pharmaceutically acceptable salt thereof according to claim 34, wherein n is 3.

36. A compound or a pharmaceutically acceptable salt thereof according to claim 34, wherein R₄ and R₅, at each occurrence, are each independently selected from the group consisting of: H, halo, hydroxyl, C₃₋₆cycloalkyl, C₁₋₄alkyl and C₁₋₄alkoxyl, which alkyl or alkoxyl groups are optionally substituted with one to three substituents independently selected from halo and C₁₋₄alkoxyl.

37. A compound or pharmaceutically acceptable salt according to claim 35, wherein A-(CR₄R₅)ₙ—O is A-CHR₄CHR₅CH₂—O, A-CR₄R₅CHR₅CH₂—O, or A-CHR₄CR₄R₅CH₂—O.

38. A compound or pharmaceutically acceptable salt according to claim 37, wherein A-(CR₄R₅)ₙ—O is A-CHR₄CHR₅CH₂—O wherein either:
R₄ and R₅ are both H; or
R₄ is H and R₅ is fluoro or C₁₋₄alkyl or C₁₋₄alkoxyl wherein said alkyl or alkoxyl is optionally substituted by one two or three fluoro groups; or
R₄ is cyclopropyl, C₁₋₄alkyl or C₁₋₄alkoxyl wherein said alkyl or alkoxyl is optionally substituted by one two or three fluoro or C₁₋₄ alkoxyl groups and R₅ is H; or
R₄ is C₁₋₄ alkyl or C₁₋₄alkoxyl wherein said alkyl or alkoxy group is optionally substituted by one two or three fluoro groups and R₅ is fluoro; or
R₄ and R₅ are both methyl.

39. A compound or pharmaceutically acceptable salt according to claim 38 wherein, A-(CR₄R₅)ₙ—O is A-CHR₄CHR₅CH₂—O wherein either:
R₄ and R₅ are both H; or
R₄ is H and R₅ is methoxyl or fluoro; or
R₄ is methyl, ethyl, fluoromethyl, difluromethyl, or methoxymethyl and R₅ is H; or
R₄ is methyl and R₅ is fluoro; or
R₄ and R₅ are both methyl.

40. A compound or a pharmaceutically acceptable salt thereof according to claim 34, having the structure of Formula (IA)

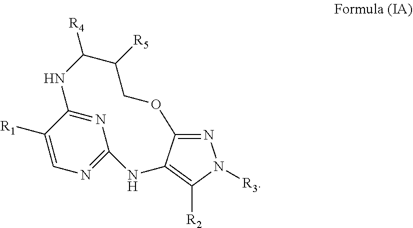

41. A compound or a pharmaceutically acceptable salt according to claim 40, wherein either:
R₄ and R₅ are both H; or
R₄ is H and R₅ is fluoro or C₁₋₄alkyl or C₁₋₄alkoxyl wherein said alkyl or alkoxyl is optionally substituted by one two or three fluoro groups; or
R₄ is cyclopropyl, C₁₋₄alkyl or C₁₋₄alkoxyl wherein said alkyl or alkoxyl is optionally substituted by one two or three fluoro or C₁₋₄ alkoxyl groups and R₅ is H; or
R₄ is C₁₋₄alkyl or C₁₋₄alkoxyl wherein said alkyl or alkoxy group is optionally substituted by one two or three fluoro groups and R₅ is fluoro; or
R₄ and R₅ are both methyl.

42. A compound or a pharmaceutically acceptable salt according to claim 41, wherein either:
R₄ and R₅ are both H; or
R₄ is H and R₅ is methoxyl or fluoro; or
R₄ is methyl, ethyl, fluoromethyl, difluromethyl, or methoxymethyl and R₅ is H; or
R₄ is methyl and R₅ is fluoro; or
R₄ and R₅ are both methyl.

43. A compound or a pharmaceutically acceptable salt according to claim 34, wherein A is NR^{a} and R^{a} is H, or C₁₋₄alkyl which C₁₋₄alkyl group is optionally substituted with one substituents independently selected from the group consisting of halo, hydroxyl and C₁₋₃alkoxyl.

44. A compound or a pharmaceutically acceptable salt according to claim 43, wherein R^{a} is H.

45. A compound or a pharmaceutically acceptable salt according to claim 34, wherein X is N.

46. A compound or a pharmaceutically acceptable salt according to claim 34, wherein R₁ is selected from the group consisting of H, halo, CN, methyl, isopropyl, tert-butyl, methoxy, trifluoromethyl, trifluoromethoxyl, ethenyl, prop-1-en-2-yl, ethynyl and cyclopropyl,

47. A compound or a pharmaceutically acceptable salt according to claim 46, wherein R₁ is selected from the group consisting of Br, Cl and CN.

48. A compound or a pharmaceutically acceptable salt according to claim 34, wherein R₂ is selected from the group consisting of H, halo, CN, methyl, ethyl, difluoromethyl, trifluoromethyl, cyclopropyl, methoxymethyl and methoxyethyl.

49. A compound or a pharmaceutically acceptable salt according to claim 48, wherein R₂ is selected from the group consisting of Cl, CN and methyl.

50. A compound or a pharmaceutically acceptable salt according to claim 34, wherein R₃ is selected from the group consisting of:
1) H;
2) four to six-membered oxygen-containing heterocyclyl ring, which heterocyclyl ring is optionally substituted with one to three substituents independently selected from the group consisting of halo; cyano and C₁₋₃alkyl which alkyl group is optionally substituted with one to three substituents independently selected from halo, hydroxyl and C₁₋₃alkoxyl;
3) four to six-membered nitrogen-containing heterocyclyl ring, which heterocycyl ring is:
substituted on the nitrogen ring atom by a substituent selected from the group consisting of an C₁₋₃alkyl group which alkyl group is optionally substituted with one to three substituents independently selected from halo, hydroxyl and C₁₋₃alkoxyl, and a four to six-membered heterocyclyl ring having one to two heteroatom ring members independently selected from O and N; and
optionally further substituted with one or two groups independently selected from halo and C₁₋₃alkyl.
4) C₁₋₆alkyl optionally substituted with one CN group; and
5) C₄₋₆cycloalkyl optionally substituted with one to three substituents independently selected from hydroxyl, morpholin-4-yl or

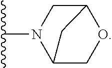 [SMILES: *N1CC2CC1CO2]

51. A method for treating Parkinson's disease, Alzheimer's disease or amyotrophic lateral sclerosis (ALS), which comprises administering to a subject in need thereof a therapeutically effective amount of a compound or a pharmaceutically acceptable salt as defined in claim 34.

52. A method according to claim 51, wherein the subject is a human.

53. A pharmaceutical composition comprising a compound or a pharmaceutically acceptable salt as defined in claim 34 and one or more pharmaceutically acceptable excipients.
